(19) United States
(12) Patent Application Publication
(10) Pub. No.: US 2022/0267352 A1
(43) Pub. Date: Aug. 25, 2022

(54) RAPAMYCIN ANALOGS AND USES THEREOF

(71) Applicant: Anakuria Therapeutics, Inc., Cambridge, MA (US)

(72) Inventors: David John O'NEILL, Arlington, MA (US); Eddine SAIAH, Brookline, MA (US); Seong Woo Anthony KANG, Somerville, MA (US)

(21) Appl. No.: 17/648,750

(22) Filed: Jan. 24, 2022

Related U.S. Application Data
(60) Provisional application No. 63/140,523, filed on Jan. 22, 2021.
(60) Provisional application No. 63/202,524, filed on Jun. 15, 2021.

(51) Int. Cl. C07D 498/16

(52) U.S. Cl. CPC C07D 498/16

(57) ABSTRACT

The present invention provides compounds, compositions thereof, and methods of using the same.

### CROSS-REFERENCE TO RELATED APPLICATIONS

This application claims the benefit under 35 U.S.C. § 119(e) to U.S. Provisional Appl. No. 63/140,523, filed Jan. 22, 2021, and U.S. Provisional Appl. No. 63/202,524, filed Jun. 15, 2021, the disclosures of each of which are herein incorporated by reference in their entireties.

### TECHNICAL FIELD OF THE INVENTION

The present invention relates to compounds and methods useful for modulating mTORC1 activity. The invention also provides pharmaceutically acceptable compositions comprising provided compounds of the present invention and methods of using such compositions in the treatment of various disorders.

### BACKGROUND OF THE INVENTION

mTOR complex 1 (mTORC1) positively regulates cell growth and proliferation by promoting many anabolic processes, including biosynthesis of proteins, lipids and organelles, and by limiting catabolic processes such as autophagy. Much of the knowledge about mTORC1 function comes from the use of the bacterial macrolide rapamycin. Upon entering the cell, rapamycin binds to FK506-binding protein of 12 kDa (FKBP12) and interacts with the FKBP12-rapamycin binding domain (FRB) of mTOR, thus inhibiting mTORC1 functions (Guertin, D.A. & Sabatini, D. M. Cancer Cell 12(1): 9-22 (2007)). In contrast to its effect on mTORC1, FKBP12-rapamycin cannot physically interact with or acutely inhibit mTOR complex 2 (mTORC2) (Janinto, E. et al., Nat. Cell Bio., 6(11): 1122-8 (2004); Sarbassov, D. D. et al., Curr. Biol. 14(14): 1296-302 (2004)). On the basis of these observations, mTORC1 and mTORC2 have been respectively characterized as the rapamycin-sensitive and rapamycin-insensitive complexes. However, this paradigm might not be entirely accurate, as chronic rapamycin treatment can, in some cases, inhibit mTORC2 activity by blocking its assembly (Sarbassov, D. D. et al., Mol. Cell, 22(2): 159-68 (2006)). In addition, recent reports suggest that important mTORC1 functions are resistant to inhibition by rapamycin (Choo, A. Y. et al., Proc. Natl. Acad. Sci., 105(45): 17414-9 (2008); Feldman, M. E. et al., PLoS Biol., 7(2):e38 (2009); Garcia-Martinez, J. M. et al., Biochem J., 421(1): 29-42 (2009); Thoreen, C. C. et al., J. Biol. Chem., 284(12): 8023-32 (2009)). Therefore, selective inhibition of mTORC1 would enable the treatment of diseases that involve dysregulation of protein synthesis and cellular metabolism. Furthermore, this detailed understanding of regulating mTORC1 activation pathways will permit the discovery of new strategies for regulating abnormal disease processes by modulating mTORC1 activity across its spectrum of function.

Many diseases are associated with abnormal cellular responses triggered by events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases.

The mechanistic target of rapamycin complex 1 (mTORC1) is a master growth regulator that senses diverse environmental cues, such as growth factors, cellular stresses, and nutrient and energy levels. When activated, mTORC1 phosphorylates substrates that potentiate anabolic processes, such as mRNA translation and lipid synthesis, and limits catabolic ones, such as autophagy. mTORC1 dysregulation occurs in a broad spectrum of diseases, including diabetes, epilepsy, neurodegeneration, immune response, suppressed skeletal muscle growth, and cancer among others (Howell, J. J. et al., Biochem. Soc. Trans., 41: 906-12 (2013); Kim, S. G. et al., Molecular and cells, 35(6): 463-73 (2013); Laplante, M. & Sabatini, D. M., Cell, 149(2): 274-93 (2012)).

Rapamycin was initially discovered as an antifungal metabolite produced by Streptomyces hygroscopicus from a soil sample of Easter Island. Subsequently, rapamycin was found to possess immunosuppressive and antiproliferative properties in mammalian cells, spurring an interest in identifying the mode of action of rapamycin. Rapamycin was shown to be a potent inhibitor of S6K1 phosphorylation. Concurrently, the target of rapamycin (TOR) was identified in yeast and animal cells. Rapamycin forms a gain-of-function complex with the 12 kDa FK506-binding protein (FKBP12), and this complex binds and specifically acts as an allosteric inhibitor of mammalian TOR (mTOR, also known as mechanistic TOR) complex 1 (mTORC1).

Biochemical and genetic analysis of mTOR has demonstrated that it is present in two functionally distinct complexes. The core components of mTORC1 consist of mTOR, mammalian lethal with sec-13 protein 8 (mLST8), and regulatory-associated protein of TOR (Raptor). Additional components include DEP-domain-containing mTOR-interacting protein (DEPTOR) and Proline-rich Akt substrate 40 kDa (PRAS40).

The mTOR complex 2 (mTORC2) core is composed of mTOR, rapamycin insensitive companion of mTOR (Rictor), stress-activated protein kinase-interacting protein 1 (mSIN1), and mLST8. Protein observed with rictor 1/2 (protor 1/2) and DEPTOR are additional regulatory components. S6 kinase 1 (S6K1) and eukaryotic inhibition factor eIF4E binding protein 1 (4E-BP1) are two well-characterized substrates of mTORC1 while AKT is a well characterized substrate of mTORC2 (Li, J. et al., Cell Met., 19(3):373-9 (2014)).

Because FKBP12-rapamycin does not bind to mTORC2, rapamycin was initially thought to inhibit only mTORC1 (Sarbassov, D. D. et al., Curr. Biol., 14(14): 1296-302 (2004)). However, in 2006 it was shown that rapamycin suppresses the assembly and function of mTORC2 and inhibits pAkt (Sarbassov, D. D. et al., Molecular Cell, 22(2): 159-68 (2006)). The effects of rapamycin on the phosphorylation of S473 of Akt (an mTORC2 substrate) and of T389 of S6K1 (an mTORC1 substrate) were compared in multiple cell lines. In PC3, HEK-293T, HeLa, and H460 cells, 1 or 24 hour treatments with rapamycin inhibited S6K1 phosphorylation, consistent with inhibition of mTORC1. Selective inhibition of S6K1 by rapamycin should lead to an increase in Akt phosphorylation, and, indeed, this is what is reported in HeLa cells. However, in PC3 cells, the drug strongly decreased Akt phosphorylation suggesting that rapamycin is not selective in this cell line. Partial inhibition of pAKT is observed in HEK-293T cells. In about one third of the cell lines, rapamycin caused a strong or partial inhibition of Akt phosphorylation, while the drug either did not affect or increased Akt phosphorylation in the others. The inhibition of pAKT after 24 hours is also observed in primary and non-transformed cell lines including endothelial and muscle cells. Rapamycin was also shown to inhibit pAkt in vivo, as mice treated daily for 1 week with the drug had decreased Akt phosphorylation in the thymus, adipose tissue, heart, and lung. These findings demonstrated that inhibition of Akt phosphorylation by rapamycin is common and occurs in normal cell lines, cancer cell lines as well as in vivo.

It was concluded by Sarbassov et al. that rapamycin and its analogs (CCI 779, RAD001 also known as everolimus, AP23573) can inhibit mTORC2 function in certain cell lines and tissues. Rapamycin-mediated inhibition of Akt may help explain the side effects of the drug. For example, rapamycin strongly inhibits Akt phosphorylation in adipose tissue, a tissue type in which insulin-stimulated Akt activity plays an important role in suppressing lipolysis. Inhibition of Akt by rapamycin in adipocytes may allow lipolysis to remain high even in the presence of insulin, resulting in the accumulation of free fatty acids in the plasma that can be used by the liver to generate triglycerides, providing a molecular mechanism for the hyperlipidemia commonly seen in patients treated with rapamycin.

Pereira et al. (Mol Cell Endocrinol., 355(1): 96-105 (2012)) explored rapamycin effects on glucose uptake and insulin signaling proteins in adipocytes obtained via fat biopsies in human donors. At therapeutic concentration (0.01 M) rapamycin reduced AKT (PKB) Ser473 phosphorylation and reduced glucose uptake in human adipocytes through impaired insulin signaling.

Lamming et al. (Science., 335(6076): 1638-1643 (2012)) demonstrated that rapamycin disrupted mTORC2 in vivo and that mTORC2 was required for the insulin-mediated suppression of hepatic gluconeogenesis.

Similar results were shown in human. Di Paolo et al. published similar findings in human (JASN, 17(8): 2236-2244 (2006)). The main objective of their study was to ascertain the effect of chronic exposure to rapamycin on AKT activation, in view of its crucial role in the regulation of cell growth and survival, as well as in the cell response to nutrients and growth factors. They found that mTOR inhibition was associated with a marked downregulation of basal and insulin-induced AKT phosphorylation. AKT is responsible primarily for many of the metabolic actions of insulin and they concluded therefore that the depression of AKT activation significantly correlated with the increase of insulin resistance in renal transplant recipients.

Kennedy et al. reviewed recently the role of mTORC1 and mTORC2 in metabolism and aging (Cell Metab., 23(6): 990-1003 (2016)).

Thus, there remains a need to provide potent and mTORC1 specific inhibitors with improved safety and tolerability due to lack of direct mTORC2 inhibition.

### SUMMARY OF THE INVENTION

It has now been found that compounds of this invention, and pharmaceutically acceptable compositions thereof, are effective as inhibitors mTORC1 inhibitors. Such compounds have the general formula I:

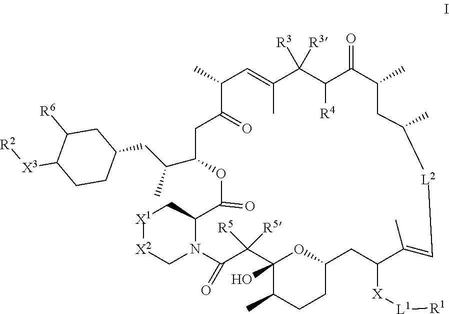

or a pharmaceutically acceptable salt thereof, wherein each of R¹, R², R³, R^{3′}, R⁴, R⁵, R^{5′}, R⁶, L¹, L², X, X¹, X², and X³ is as defined and described herein.

Compounds of the present invention, and pharmaceutically acceptable compositions thereof, are useful for treating a variety of diseases, disorders or conditions, associated with mTORC1. Such diseases, disorders, or conditions include those described herein.

### BRIEF DESCRIPTION OF THE DRAWINGS

FIG. 1 shows a comparison of Western blots preformed after treating PC3 cells with rapamycin, I-59, I-57, and I-55 for 24 hours.

FIG. 2 shows a comparison of Western blots preformed after treating PC3 cells with rapamycin, I-69, I-66, I-64, and I-62 for 24 hours.

FIG. 3 shows a comparison of Western blots preformed after treating PC3 cells with rapamycin, I-85, I-97, and I-83 for 24 hours.

FIG. 4 shows a comparison of Western blots preformed after treating PC3 cells with rapamycin, I-34, I-49, and I-31 for 24 hours.

FIG. 5 shows a comparison of Western blots preformed after treating PC3 cells with rapamycin, I-37, I-43, and I-40 for 24 hours.

FIG. 6 shows a comparison of Western blots preformed after treating PC3 cells with rapamycin and I-14 for 24 hours.

FIG. 7 shows a comparison of Western blots preformed after treating PC3 cells with rapamycin, I-4, I-27, and I-47 for 24 hours.

FIG. 8 shows a comparison of Western blots preformed after treating PC3 cells with rapamycin, I-9, and I-21 for 24 hours.

FIG. 9 shows a comparison of Western blots preformed after treating PC3 cells with rapamycin, I-18, and I-45 for 24 hours.

### DETAILED DESCRIPTION OF CERTAIN EMBODIMENTS

### 1. General Description of Certain Embodiments of the Invention

It has been surprisingly found that provided compounds inhibit mTORC1, but do not impact mTORC2 (as measured by their impact on pAKT) over extended periods of time (e.g., 8 hours, 24 hours, 30 hours, and 48 hours). This novel activity is predicated on the presence of a sufficiently large group at the C-7 position of rapamycin and its analogs. Small substitutions at this position such as OMe, as seen in rapamycin, OEt, OBn do not confer selectivity over mTORC2 at 24 hours. Medium length groups, such as OCH₂CH₂OH or OCH₂CH₂CH₂OH show partial selectivity over mTORC2 at 24 hours, but still show some level of inhibition. In comparison, larger groups, such as those of the present invention, provide a marked selectivity over mTORC2 as measured by the impact of pAKT.

The location of this substitution is also critical to the observed selectivity. Introduction of larger substitutions at C-43 position for example does not lead to this unique selectivity profile claimed in this application.

For the purpose of clarity, the structure of Rapamycin is reproduced below with the C-7 and C-43 positions noted.

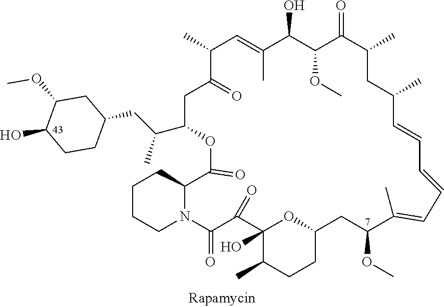 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

In some embodiments, the present invention provides novel rapamycin analogues that are potent mTORC1 inhibitors as measured by pS6K. Unlike Rapamycin and Everolimus, these compounds do not inhibit pAKT at longer time points (e.g., 24 hours and 48 hours). These compounds also show improved solubility and improved pharmacokinetics comparing to Rapamycin.

The activity of a compound utilized in this invention as an inhibitor of mTORC1, may be assayed in vitro, in vivo or in a cell line. In vitro assays include assays that determine the inhibition of mTORC1. Detailed conditions for assaying a compound utilized in this invention as an inhibitor of mTORC1 are well known to one of ordinary skill in the art. Such methods are described in detail by Liu et al., Cancer Research, 73(8): 2574-86 (2013) and Liu et al., J. Biological Chemistry 287(13): 9742-52 (2012).

In certain embodiments, the present invention provides a compound of formula I:

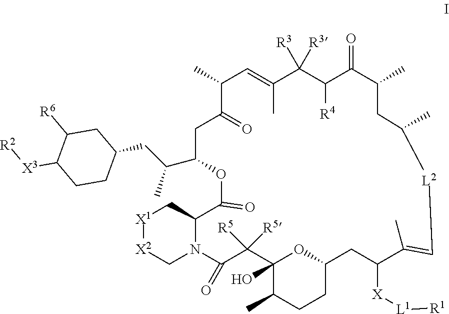

or a pharmaceutically acceptable salt thereof, wherein:
X and X³ are independently a covalent bond, —CR₂—, —NR—, —NRCO—, —NRCO₂—, —NRCONR—, —NRSO₂—, —O—, —S—, or —SO₂NR—;
L¹ is a covalent bond or a C₁₋₃₀ bivalent straight or branched saturated or unsaturated hydrocarbon chain, wherein 1-10 methylene units of the chain are independently and optionally replaced with -Cy₁-, —O—, —S—, —S(O)₂—, —C(O)—, —C(S)—, —C(R)₂—, —CH(R)—, —CF₂—, —P(O)(R)—, —Si(R)₂—, —Si(OR)(R)—, or —NR—;
each -Cy₁- is independently an optionally substituted bivalent ring selected from phenylene, 4-7 membered saturated or partially unsaturated heterocyclylene having 1-2 heteroatoms independently selected from nitrogen, oxygen, and sulfur, and 5-6 membered heteroarylene having 1-4 heteroatoms independently selected from nitrogen, oxygen, and sulfur;
each R is independently hydrogen, or an optionally substituted group selected from C₁₋₆ aliphatic, 3-8 membered saturated or partially unsaturated monocyclic carbocyclic ring, phenyl, 4-7 membered saturated or partially unsaturated heterocyclic ring having 1-2 heteroatoms independently selected from nitrogen, oxygen, and sulfur, and 5-6 membered heteroaryl ring having 1-4 heteroatoms independently selected from nitrogen, oxygen, and sulfur, or
   two R groups on the same atom are taken together with their intervening atoms to form a 4-7 membered saturated, partially unsaturated, or aryl ring having 0-3 heteroatoms, in addition to the same atom to which they are attached, independently selected from nitrogen, oxygen, and sulfur;
L² is an optionally substituted C₁₋₆ bivalent straight or branched saturated or unsaturated hydrocarbon chain, wherein 1-2 methylene units of the chain are independently and optionally replaced with -Cy₁-;
R¹ and R² are independently hydrogen, halogen, —OR, —CN, —NO₂, —NR₂, —NR(C₁₋₆ haloalkyl), —NRCOR, —NRCO₂R, —NRCONR₂, —NRSO₂R, —SR, —SO₂NR₂, or an optionally substituted group selected from C₁₋₆ aliphatic, 3-8 membered saturated or partially unsaturated monocyclic carbocyclic ring, phenyl, 8-10 membered bicyclic aromatic carbocyclic ring, 4-8 membered saturated or partially unsaturated monocyclic heterocyclic ring having 1-2 heteroatoms independently selected from nitrogen, oxygen, and sulfur, 7-10 membered saturated or partially unsaturated bicyclic heterocyclic ring having 1-3 heteroatoms independently selected from nitrogen, oxygen, and sulfur, 5-6 membered monocyclic heteroaromatic ring having 1-4 heteroatoms independently selected from nitrogen, oxygen, and sulfurs, and 8-10 membered bicyclic heteroaromatic ring having 1-5 heteroatoms independently selected from nitrogen, oxygen, and sulfur;
R³ is hydrogen, halogen; —OR, or —OSiR₃;
R^{3′} is hydrogen, halogen; —OR, or —OSiR₃,
   or R³ and R^{3′} are taken together to form ═O or ═S;
R⁴ and R⁶ are independently hydrogen, —OR, —NR₂, —NRCOR, —NRCO₂R, —NRCONR₂, —NRSO₂R, —SR, —SO₂NR₂, or an optionally substituted C₁₋₆ aliphatic;
R⁵ and R^{5′} are each hydrogen or taken together to form ═O or ═NOR;
X¹ and X² are each independently —CR₂—, —S—, or —S(O)—,
wherein at least one of X¹ and X² is —CR₂—.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic or bicyclic (e.g., bridged bicyclic or spirocyclic). The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}; —N(R^{∘})C(S)NR^{∘}₂; —(CH₂)_{0- 4}N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₄ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C1-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, compounds having the structures depicted herein are also meant to comprise any pharmaceutically acceptable salts, stereoisomers, tautomers, solvates, hydrates and polymorphs thereof.

Unless otherwise stated, compounds having the structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

The terms “measurable affinity” and “measurably inhibit,” as used herein, means a measurable change in mTORC1 activity between a sample comprising a compound of the present invention, or composition thereof, and mTORC1, and an equivalent sample comprising mTORC1 in the absence of said compound, or composition thereof.

### 3. Description of Exemplary Embodiments

As described above, in certain embodiments, the present invention provides a compound of formula I:

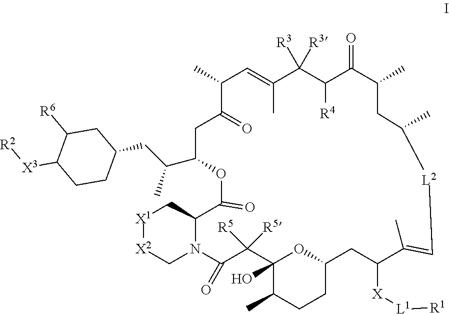

or a pharmaceutically acceptable salt thereof, wherein:
X and X³ are independently a covalent bond, —CR₂—, —NR—, —NRCO—, —NRCO₂—, —NRCONR—, —NRSO₂—, —O—, —S—, or —SO₂NR—;
L¹ is a covalent bond or a C₁₋₃₀ bivalent straight or branched saturated or unsaturated hydrocarbon chain, wherein 1-10 methylene units of the chain are independently and optionally replaced with -Cy₁-, —O—, —S—, —SO₂—, —C(O)—, —C(S)—, —CR₂—, —CF₂—, —P(O)(R)—, —SiR₂—, —Si(OR)(R)—, or —NR—;
each -Cy₁- is independently an optionally substituted bivalent ring selected from phenylene, 4-7 membered saturated or partially unsaturated heterocyclylene having 1-2 heteroatoms independently selected from nitrogen, oxygen, and sulfur, and 5-6 membered heteroarylene having 1-4 heteroatoms independently selected from nitrogen, oxygen, and sulfur;
each R is independently hydrogen, or an optionally substituted group selected from C₁₋₆ aliphatic, 3-8 membered saturated or partially unsaturated monocyclic carbocyclic ring, phenyl, 4-7 membered saturated or partially unsaturated heterocyclic ring having 1-2 heteroatoms independently selected from nitrogen, oxygen, and sulfur, and 5-6 membered heteroaryl ring having 1-4 heteroatoms independently selected from nitrogen, oxygen, and sulfur, or
   two R groups on the same atom are taken together with their intervening atoms to form a 4-7 membered saturated, partially unsaturated, or aryl ring having 0-3 heteroatoms, in addition to the same atom to which they are attached, independently selected from nitrogen, oxygen, and sulfur;
L² is an optionally substituted C₁₋₆ bivalent straight or branched saturated or unsaturated hydrocarbon chain, wherein 1-2 methylene units of the chain are independently and optionally replaced with -Cy₁-;
R¹ and R² are independently hydrogen, halogen, —OR, —CN, —NO₂, —NR₂, —NR(C₁₋₆ haloalkyl), —NRCOR, —NRCO₂R, —NRCONR₂, —NRSO₂R, —SR, —SO₂NR₂, or an optionally substituted group selected from C₁₋₆ aliphatic, 3-8 membered saturated or partially unsaturated monocyclic carbocyclic ring, phenyl, 8-10 membered bicyclic aromatic carbocyclic ring, 4-8 membered saturated or partially unsaturated monocyclic heterocyclic ring having 1-2 heteroatoms independently selected from nitrogen, oxygen, and sulfur, 7-10 membered saturated or partially unsaturated bicyclic heterocyclic ring having 1-3 heteroatoms independently selected from nitrogen, oxygen, and sulfur, 5-6 membered monocyclic heteroaromatic ring having 1-4 heteroatoms independently selected from nitrogen, oxygen, and sulfurs, and 8-10 membered bicyclic heteroaromatic ring having 1-5 heteroatoms independently selected from nitrogen, oxygen, and sulfur;
R³ is hydrogen, halogen; —OR, or —OSiR₃;
R^{3′} is hydrogen, halogen; —OR, or —OSiR₃,
   or R³ and R^{3′} are taken together to form ═O or ═S;
R⁴ and R⁶ are independently hydrogen, —OR, —NR₂, —NRCOR, —NRCO₂R, —NRCONR₂, —NRSO₂R, —SR, —SO₂NR₂, or an optionally substituted C₁₋₆ aliphatic;
R⁵ and R^{5′} are each hydrogen or taken together to form ═O or ═NOR;
X¹ and X² are each independently —CR₂—, —S—, or —S(O)—,
wherein at least one of X¹ and X² is —CR₂—.

In certain embodiments, the present invention provides a compound of formula I′:

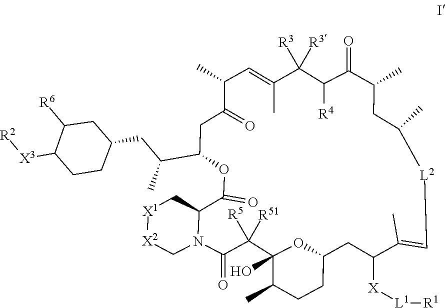

or a pharmaceutically acceptable salt thereof, wherein:
X and X³ are independently a covalent bond, —CR₂—, —NR—, —NRCO—, —NRCO₂—, —NRCONR—, —NRSO₂—, —O—, —S—, or —SO₂NR—;
L¹ is a covalent bond or a C₁-30 bivalent straight or branched saturated or unsaturated hydrocarbon chain, wherein 1-10 methylene units of the chain are independently and optionally replaced with -Cy₁-, —O—, —S—, —SO₂—, —C(O)—, —C(S)—, —CR₂—, —CF₂—, —P(O)(R)—, —SiR₂—, —Si(OR)(R)—, or —NR—;
each -Cy₁- is independently an optionally substituted bivalent ring selected from phenylene, 4-7 membered saturated or partially unsaturated heterocyclylene having 1-2 heteroatoms independently selected from nitrogen, oxygen, and sulfur, and 5-6 membered heteroarylene having 1-4 heteroatoms independently selected from nitrogen, oxygen, and sulfur;
each R is independently hydrogen, or an optionally substituted group selected from C₁₋₆ aliphatic, 3-8 membered saturated or partially unsaturated monocyclic carbocyclic ring, phenyl, 4-7 membered saturated or partially unsaturated heterocyclic ring having 1-2 heteroatoms independently selected from nitrogen, oxygen, and sulfur, and 5-6 membered heteroaryl ring having 1-4 heteroatoms independently selected from nitrogen, oxygen, and sulfur, or
   two R groups on the same atom are taken together with their intervening atoms to form a 4-7 membered saturated, partially unsaturated, or aryl ring having 0-3 heteroatoms, in addition to the same atom to which they are attached, independently selected from nitrogen, oxygen, and sulfur;
L² is an optionally substituted C₁₋₆ bivalent straight or branched saturated or unsaturated hydrocarbon chain, wherein 1-2 methylene units of the chain are independently and optionally replaced with -Cy₁-;
R¹ and R² are independently hydrogen, halogen, —OR, —CN, —(CR₂)₁₋₄NR₂, —COR, —CONR₂, —CONR(CR₂)₁₋₄NR₂, —NO₂, —NR₂, —NR(C₁₋₆ haloalkyl), —NRCOR, —NRCO₂R, —NRCONR₂, —NRSO₂R, —SR, —SO₂NR₂, —P(O)R₂, or an optionally substituted group selected from C₁₋₆ aliphatic, 3-8 membered saturated or partially unsaturated monocyclic carbocyclic ring, phenyl, 8-10 membered bicyclic aromatic carbocyclic ring, 4-8 membered saturated or partially unsaturated monocyclic heterocyclic ring having 1-2 heteroatoms independently selected from nitrogen, oxygen, and sulfur, 7-10 membered saturated or partially unsaturated bicyclic heterocyclic ring having 1-3 heteroatoms independently selected from nitrogen, oxygen, and sulfur, 5-6 membered monocyclic heteroaromatic ring having 1-4 heteroatoms independently selected from nitrogen, oxygen, and sulfurs, and 8-10 membered bicyclic heteroaromatic ring having 1-5 heteroatoms independently selected from nitrogen, oxygen, and sulfur;
R³ is hydrogen, halogen; —OR, or —OSiR₃;
R^{3′} is hydrogen, halogen; —OR, or —OSiR₃,
   or R³ and R^{3′} are taken together to form ═O or ═S;
R⁴ and R⁶ are independently hydrogen, —OR, —NR₂, —NRCOR, —NRCO₂R, —NRCONR₂, —NRSO₂R, —SR, —SO₂NR₂, or an optionally substituted C₁₋₆ aliphatic;
R⁵ and R^{5′} are each hydrogen or taken together to form ═O or ═NOR;
X¹ and X² are each independently —CR₂—, —S—, or —S(O)—,
wherein at least one of X¹ and X² is —CR₂—.

It will be appreciated that the term “rapamycin”, and structure thereof, recited throughout the specification is intended to encompass rapamycin and analogs thereof.

The herein recited analogs of rapamycin (i.e., rapalogs) are for exemplification and not intended to limit the current invention.

As defined above and described herein, X and X³ are independently a covalent bond, —CR₂—, —NR—, —NRCO—, —NRCO₂—, —NRCONR—, —NRSO₂—, —O—, —S—, or —SO₂NR—.

In some embodiments, X is a covalent bond. In some embodiments, X is —CR₂—. In some embodiments, X is —NR—. In some embodiments, X is —NRCO—. In some embodiments, X is —NRCO₂—. In some embodiments, X is —NRCONR—. In some embodiments, X is —NRSO₂—. In some embodiments, X is —O—. In some embodiments, X is —S—. In some embodiments, X is —SO₂NR—.

In some embodiments, X³ is a covalent bond. In some embodiments, X³ is —CR₂—. In some embodiments, X³ is —NR—. In some embodiments, X³ is —NRCO—. In some embodiments, X³ is —NRCO₂—. In some embodiments, X³ is —NRCONR—. In some embodiments, X³ is —NRSO₂—. In some embodiments, X³ is —O—. In some embodiments, X³ is —S—. In some embodiments, X³ is —SO₂NR—.

In some embodiments, wherein X is an unsymmetric group, such as —NRCO—, —NRCO₂—, —NRSO₂—, or —SO₂NR—, X binds to L¹ as —NRCOL¹-, —NRCO₂L¹-, —NRSO₂L¹-, and —SO₂NRL¹-.

In some embodiments, wherein X³ is an unsymmetric group, such as —NRCO—, —NRCO₂—, —NRSO₂—, or —SO₂NR—, X³ binds to R² as —NRCOR², —NRCO₂R², —NRSO₂R², and —SO₂NRR².

In some embodiments, X and X³ are selected from those depicted in the compounds of Table 1.

As defined above and described herein, L¹ is a covalent bond or a C₁₋₃₀ bivalent straight or branched saturated or unsaturated hydrocarbon chain, wherein 1-10 methylene units of the chain are independently and optionally replaced with -Cy₁-, —O—, —S—, —SO₂—, —C(O)—, —C(S)—, —CR₂—, —CF₂—, —P(O)(R)—, —SiR₂—, —Si(OR)(R)—, or —NR—.

In some embodiments, L¹ is a covalent bond. In some embodiments, L¹ is a C₁₋₃₀ bivalent straight or branched saturated or unsaturated hydrocarbon chain, wherein 1-10 methylene units of the chain are independently and optionally replaced with -Cy₁-, —O—, —S—, —SO₂—, —C(O)—, —C(S)—, —CR₂—, —CF₂—, —P(O)(R)—, —SiR₂—, —Si(OR)(R)—, or —NR—.

In some embodiments, L¹ is —CH₂—. In some embodiments, L¹ is —CH₂CH₂—. In some embodiments, L¹ is —(CH₂)₃—. In some embodiments, L¹ is —(CH₂)₄—. In some embodiments, L¹ is —(CH₂)₅—. In some embodiments, L¹ is —CH₂CH₂O—. In some embodiments, L¹ is —(CH₂CH₂O)₂—. In some embodiments, L¹ is —(CH₂CH₂O)₃—. In some embodiments, L¹ is —CH₂CH₂OCH₂CH₂—. In some embodiments, L¹ is —CH₂CH₂SO₂CH₂CH₂O—. In some embodiments, L¹ is —CH₂CH₂OCH₂CH₂OCH₂CH₂—. In some embodiments, L¹ is —CH₂CH₂OCH₂CH₂—.

In some embodiments, L¹ is selected from those depicted in the compounds of Table 1.

As defined above and described herein, each -Cy₁- is independently an optionally substituted bivalent ring selected from phenylene, 4-7 membered saturated or partially unsaturated heterocyclylene having 1-2 heteroatoms independently selected from nitrogen, oxygen, and sulfur, and 5-6 membered heteroarylene having 1-4 heteroatoms independently selected from nitrogen, oxygen, and sulfur.

In some embodiments, -Cy₁- is an optionally substituted bivalent ring selected from phenylene. In some embodiments, -Cy₁- is an optionally substituted 4-7 membered saturated or partially unsaturated heterocyclylene having 1-2 heteroatoms independently selected from nitrogen, oxygen, and sulfur. In some embodiments, -Cy₁- is an optionally substituted 5-6 membered heteroarylene having 1-4 heteroatoms independently selected from nitrogen, oxygen, and sulfur.

In some embodiments, -Cy₁- is

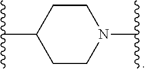 [SMILES: *C1CCN(*)CC1]

In some embodiments, -Cy₁- is selected from those depicted in the compounds of Table 1.

As defined above and described herein, each R is independently hydrogen, or an optionally substituted group selected from C₁₋₆ aliphatic, 3-8 membered saturated or partially unsaturated monocyclic carbocyclic ring, phenyl, 4-7 membered saturated or partially unsaturated heterocyclic ring having 1-2 heteroatoms independently selected from nitrogen, oxygen, and sulfur, and 5-6 membered heteroaryl ring having 1-4 heteroatoms independently selected from nitrogen, oxygen, and sulfur, or two R groups on the same atom are taken together with their intervening atoms to form a 4-7 membered saturated, partially unsaturated, or aryl ring having 0-3 heteroatoms, in addition to the same atom to which they are attached, independently selected from nitrogen, oxygen, and sulfur.

In some embodiments, R is hydrogen. In some embodiments, R is an optionally substituted C₁₋₆ aliphatic. In some embodiments, R is C₁₋₆ haloalkyl. In some embodiments, R is an optionally substituted 3-8 membered saturated or partially unsaturated monocyclic carbocyclic ring. In some embodiments, R is an optionally substituted phenyl. In some embodiments, R is an optionally substituted 4-7 membered saturated or partially unsaturated heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same atom are taken together with their intervening atoms to form an optionally substituted 4-7 membered saturated, partially unsaturated, or aryl ring having 0-3 heteroatoms, in addition to the same atom to which they are attached, independently selected from nitrogen, oxygen, and sulfur.

In some embodiments, R is selected from those depicted in the compounds of Table 1.

As defined above and described herein, L² is an optionally substituted C₁₋₆ bivalent straight or branched saturated or unsaturated hydrocarbon chain, wherein 1-2 methylene units of the chain are independently and optionally replaced with -Cy₁-.

In some embodiments, L² is an optionally substituted C₁₋₆ bivalent straight or branched saturated or unsaturated hydrocarbon chain, wherein 1-2 methylene units of the chain are independently and optionally replaced with -Cy₁-.

In some embodiments, L² is

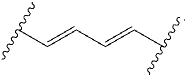 [SMILES: */C=C/C=C/*]

In some embodiments, L² is selected from those depicted in the compounds of Table 1.

As defined above and described herein, R¹ and R² are independently hydrogen, halogen, —OR, —CN, —(CR₂)₁₋₄NR₂, —COR, —CONR₂, —CONR(CR₂)₁₋₄NR₂, —NO₂, —NR₂, —NR(C₁₋₆ haloalkyl), —NRCOR, —NRCO₂R, —NRCONR₂, —NRSO₂R, —SR, —SO₂NR₂, —P(O)R₂, or an optionally substituted group selected from C₁₋₆ aliphatic, 3-8 membered saturated or partially unsaturated monocyclic carbocyclic ring, phenyl, 8-10 membered bicyclic aromatic carbocyclic ring, 4-8 membered saturated or partially unsaturated monocyclic heterocyclic ring having 1-2 heteroatoms independently selected from nitrogen, oxygen, and sulfur, 7-10 membered saturated or partially unsaturated bicyclic heterocyclic ring having 1-3 heteroatoms independently selected from nitrogen, oxygen, and sulfur, 5-6 membered monocyclic heteroaromatic ring having 1-4 heteroatoms independently selected from nitrogen, oxygen, and sulfurs, and 8-10 membered bicyclic heteroaromatic ring having 1-5 heteroatoms independently selected from nitrogen, oxygen, and sulfur.

In some embodiments, R¹ is hydrogen. In some embodiments, R¹ is halogen. In some embodiments, R¹ is —OR. In some embodiments, R¹ is —CN. In some embodiments, R¹ is —NO₂. In some embodiments, R¹ is —NR₂. In some embodiments, R¹ is —NR(C₁₋₆ haloalkyl). In some embodiments, R¹ is —NRCOR. In some embodiments, R¹ is —NRCO₂R. In some embodiments, R¹ is —NRCONR₂. In some embodiments, R¹ is —NRSO₂R. In some embodiments, R¹ is —SR. In some embodiments, R¹ is —SO₂NR₂. In some embodiments, R¹ is an optionally substituted C₁₋₆ aliphatic. In some embodiments, R¹ is an optionally substituted 3-8 membered saturated or partially unsaturated monocyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 4-8 membered saturated or partially unsaturated monocyclic heterocyclic ring having 1-2 heteroatoms independently selected from nitrogen, oxygen, and sulfur. In some embodiments, R¹ is an optionally substituted 7-10 membered saturated or partially unsaturated bicyclic heterocyclic ring having 1-3 heteroatoms independently selected from nitrogen, oxygen, and sulfur. In some embodiments, R¹ is an optionally substituted 5-6 membered monocyclic heteroaromatic ring having 1-4 heteroatoms independently selected from nitrogen, oxygen, and sulfurs. In some embodiments, R¹ is an optionally substituted 8-10 membered bicyclic heteroaromatic ring having 1-5 heteroatoms independently selected from nitrogen, oxygen, and sulfur.

In some embodiments, R¹ is methyl. In some embodiments, R¹ is —NH₂. In some embodiments, R¹ is —NHMe. In some embodiments, R¹ is —NMe₂. In some embodiments, R¹ is —CH₂CF₃. In some embodiments, R¹ is —SO₂—NH₂. In some embodiments, R¹ is —CONH₂. In some embodiments, R¹ is —CONMe₂. In some embodiments, R¹ is —OCONHMe. In some embodiments, R¹ is —CO₂H. In some embodiments, R¹ is

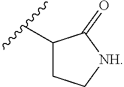 [SMILES: *C1CCNC1=O]

In some embodiments, R¹ is

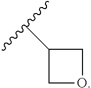 [SMILES: *C1COC1]

In some embodiments, R¹ is

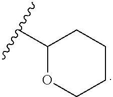 [SMILES: *C1CCCCO1]

In some embodiments, R¹ is

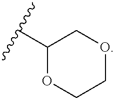 [SMILES: *C1COCCO1]

In some embodiments, R¹ is

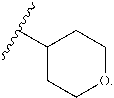 [SMILES: *C1CCOCC1]

In some embodiments, R¹ is

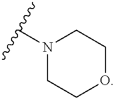 [SMILES: *N1CCOCC1]

In some embodiments, R¹ is

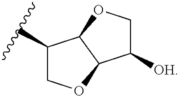 [SMILES: *[C@@H]1CO[C@H]2[C@@H]1OC[C@H]2O]

In some embodiments, R¹ is

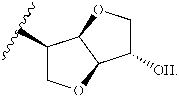 [SMILES: *[C@@H]1CO[C@H]2[C@@H]1OC[C@@H]2O]

In some embodiments, R¹ is

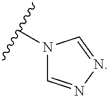 [SMILES: *n1cnnc1]

some embodiments, R¹ is

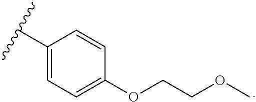 [SMILES: *c1ccc(OCCOC)cc1]

In some embodiments, R¹ is

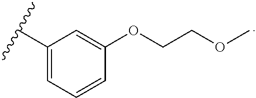 [SMILES: *c1cccc(OCCOC)c1]

In some embodiments, R¹ is

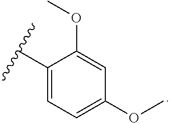 [SMILES: *c1ccc(OC)cc1OC]

In some embodiments, R¹ is

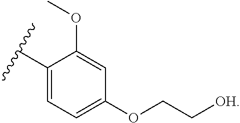 [SMILES: *c1ccc(OCCO)cc1OC]

In some embodiments R¹ is

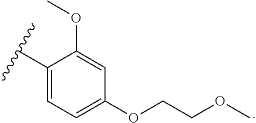 [SMILES: *c1ccc(OCCOC)cc1OC]

In some embodiments, R¹ is

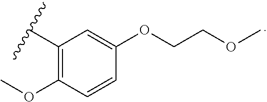

In some embodiments, R¹ is

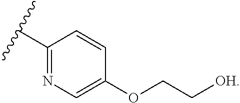 [SMILES: *c1ccc(OCCO)cn1]

In some embodiments, R¹ is

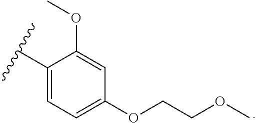 [SMILES: *c1ccc(OCCOC)cc1OC]

In some embodiments, R¹ is

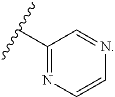 [SMILES: *c1cnccn1]

In some embodiments, R¹ is

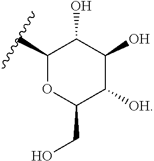 [SMILES: *[C@@H]1O[C@H](CO)[C@@H](O)[C@H](O)[C@H]1O]

In some embodiments, R¹ is

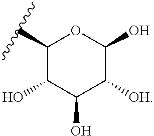 [SMILES: *[C@H]1O[C@@H](O)[C@H](O)[C@@H](O)[C@@H]1O]

In some embodiments, R¹ is

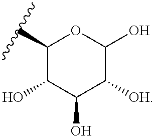 [SMILES: *[C@H]1OC(O)[C@H](O)[C@@H](O)[C@@H]1O]

In some embodiments, R¹ is

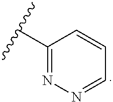 [SMILES: *c1cccnn1]

In some embodiments, R¹ is

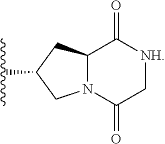 [SMILES: *[C@@H]1C[C@H]2C(=O)NCC(=O)N2C1]

In some embodiments, R¹ is

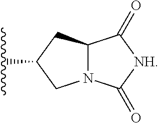 [SMILES: *[C@@H]1C[C@H]2C(=O)NC(=O)N2C1]

In some embodiments, R¹ is

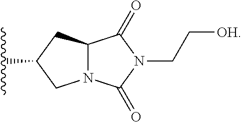 [SMILES: *[C@@H]1C[C@H]2C(=O)N(CCO)C(=O)N2C1]

In some embodiments, R¹ is

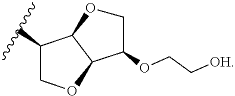 [SMILES: *[C@@H]1CO[C@H]2[C@@H]1OC[C@H]2OCCO]

In some embodiments, R¹ is

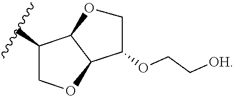 [SMILES: *[C@@H]1CO[C@H]2[C@@H]1OC[C@@H]2OCCO]

In some embodiments, R¹ is

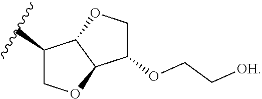 [SMILES: *[C@@H]1CO[C@H]2[C@H]1OC[C@@H]2OCCO]

In some embodiments, R¹ is

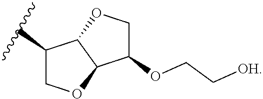 [SMILES: *[C@@H]1CO[C@H]2[C@H]1OC[C@H]2OCCO]

In some embodiments, R² is hydrogen. In some embodiments, R² is halogen. In some embodiments, R¹² is —OR. In some embodiments, R² is —CN. In some embodiments, R² is —(CR₂)₁₋₄NR₂. In some embodiments, R² is —COR. In some embodiments, R² is —CONR₂. In some embodiments, R² is —CONR(CR₂)₁₋₄NR₂. In some embodiments, R² is —NO₂. In some embodiments, R² is —NR₂. In some embodiments, R² is —NR(C₁₋₆ haloalkyl). In some embodiments, R² is —NRCOR. In some embodiments, R² is —NRCO₂R. In some embodiments, R² is —NRCONR₂. In some embodiments, R² is —NRSO₂R. In some embodiments, R² is —SR. In some embodiments, R² is —SO₂NR₂. In some embodiments, R² is —P(O)R₂. In some embodiments, R² is an optionally substituted C₁₋₆ aliphatic. In some embodiments, R² is an optionally substituted 3-8 membered saturated or partially unsaturated monocyclic carbocyclic ring. In some embodiments, R² is an optionally substituted phenyl. In some embodiments, R² is an optionally substituted 8-10 membered bicyclic aromatic carbocyclic ring. In some embodiments, R² is an optionally substituted 4-8 membered saturated or partially unsaturated monocyclic heterocyclic ring having 1-2 heteroatoms independently selected from nitrogen, oxygen, and sulfur. In some embodiments, R² is an optionally substituted 7-10 membered saturated or partially unsaturated bicyclic heterocyclic ring having 1-3 heteroatoms independently selected from nitrogen, oxygen, and sulfur. In some embodiments, R² is an optionally substituted 5-6 membered monocyclic heteroaromatic ring having 1-4 heteroatoms independently selected from nitrogen, oxygen, and sulfurs. In some embodiments, R² is an optionally substituted 8-10 membered bicyclic heteroaromatic ring having 1-5 heteroatoms independently selected from nitrogen, oxygen, and sulfur.

In some embodiments, R² is methyl. In some embodiments, R² is —CHF₂. In some embodiments, R² is —CF₃. In some embodiments, R² is

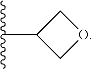 [SMILES: *C1COC1]

In some embodiments, R² is

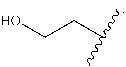 [SMILES: *CCO]

In some embodiments, R² is

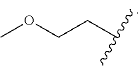 [SMILES: *CCOC]

In some embodiments, R² is

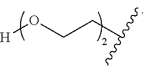

In some embodiments, R² is

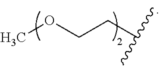

In some embodiments, R² is

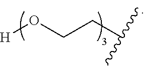

In some embodiments, R² is

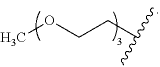

In some embodiments, R² is

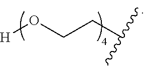

In some embodiments, R² is

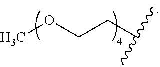

In some embodiments, R² is

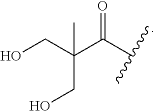 [SMILES: *C(=O)C(C)(CO)CO]

In some embodiments, R² is

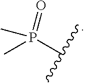 [SMILES: *P(C)(C)=O]

In some embodiments, R² is

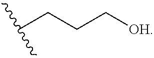 [SMILES: *CCCO]

In some embodiments, R² is

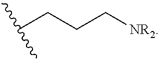

In some embodiments, R² is

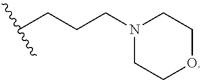 [SMILES: *CCCN1CCOCC1]

In some embodiments, R² is

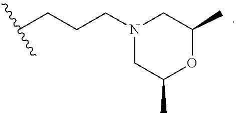 [SMILES: *CCCN1C[C@@H](C)O[C@@H](C)C1]

In some embodiments, R² is

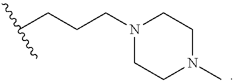 [SMILES: *CCCN1CCN(C)CC1]

In some embodiments, R² is

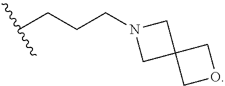 [SMILES: *CCCN1CC2(COC2)C1]

In some embodiments, R² is

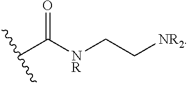

In some embodiments, R² is

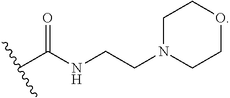 [SMILES: *C(=O)NCCN1CCOCC1]

In some embodiments, R² is

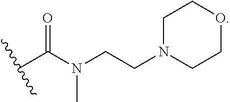 [SMILES: *C(=O)N(C)CCN1CCOCC1]

In some embodiments, R¹ and R² are selected from those depicted in the compounds of Table 1.

As defined above and described herein, R³ is hydrogen, halogen, —OR, or —OSiR₃.

In some embodiments, R³ is hydrogen. In some embodiments, R³ is halogen. In some embodiments, R³ is —OR. In some embodiments, R³ is —OSiR₃.

In some embodiments, R³ is —OMe.

As defined above and described herein, R^{3′} is hydrogen, halogen, —OR, or —OSiR₃.

In some embodiments, R³ is hydrogen. In some embodiments, R³ is halogen. In some embodiments, R³ is —OR. In some embodiments, R³ is —OSiR₃.

As defined above and described herein, in some embodiments, R³ and R^{3′} are taken together to form ═O or ═S;

In some embodiments, R³ and R^{3′} are taken together to form ═O. In some embodiments, R³ and R^{3′} are taken together to form ═S.

In some embodiments, R³ and R^{3′} are selected from those depicted in the compounds of Table 1.

As defined above and described herein, R⁴ and R⁶ are independently hydrogen, —OR, —NR₂, —NRCOR, —NRCO₂R, —NRCONR₂, —NRSO₂R, —SR, —SO₂NR₂, or an optionally substituted C₁₋₆ aliphatic.

In some embodiments, R⁴ is hydrogen. In some embodiments, R⁴ is —OR. In some embodiments, R⁴ is —NR₂. In some embodiments, R⁴ is —NRCOR. In some embodiments, R⁴ is —NRCO₂R. In some embodiments, R⁴ is —NRCONR₂. In some embodiments, R⁴ is —NRSO₂R. In some embodiments, R⁴ is —SR. In some embodiments, R⁴ is —SO₂NR₂. In some embodiments, R⁴ is an optionally substituted C₁₋₆ aliphatic.

In some embodiments, R⁴ is —OH. In some embodiments, R⁴ is —OMe.

In some embodiments, R⁶ is hydrogen. In some embodiments, R⁶ is —OR. In some embodiments, R⁶ is —NR₂. In some embodiments, R⁶ is —NRCOR. In some embodiments, R⁶ is —NRCO₂R. In some embodiments, R⁶ is —NRCONR₂. In some embodiments, R⁶ is —NRSO₂R. In some embodiments, R⁶ is —SR. In some embodiments, R⁶ is —SO₂NR₂. In some embodiments, R⁶ is an optionally substituted C₁₋₆ aliphatic.

In some embodiments, R⁶ is —OMe.

In some embodiments, R⁴ and R⁶ are selected from those depicted in the compounds of Table 1.

As defined above and described herein, R⁵ and R^{5′} are each hydrogen or taken together to form ═O or ═NOR.

In some embodiments, R⁵ is hydrogen. In some embodiments, R^{5′} is hydrogen. In some embodiments, R⁵ and R^{5′} are taken together to form ═O. In some embodiments, R⁵ and R^{5′} are taken together to form ═NOR.

In some embodiments, R⁵ and R^{5′} are selected from those depicted in the compounds of Table 1.

As defined above and described herein, X¹ and X² are each independently —CR₂—, —S—, or —S(O)—, wherein at least one of X¹ and X² is —CR₂—.

In some embodiments, X¹ is —CR₂—. In some embodiments, X¹ is —S—. In some embodiments, X¹ is —S(O)—. In some embodiments, X² is —CR₂—. In some embodiments, X² is —S—. In some embodiments, X² is —S(O)—.

In some embodiments, X¹ and X² are selected from those depicted in the compounds of Table 1.

In certain embodiments, the present invention provides a compound of formula I or I′ wherein X¹ is —CH₂—, X² is —CH₂—, and L² is

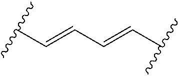 [SMILES: */C=C/C=C/*]

as shown below, thereby providing a compound of formula I-a-1:

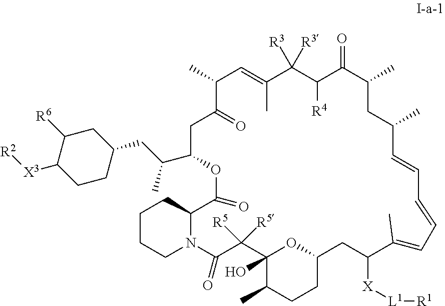

or a pharmaceutically acceptable salt thereof, wherein:
X is —CR₂—, —NRCO—, —NRCO₂—, —NRCONR—, —NRSO₂—, or —SO₂NR—; and
each of X³, R¹, R², R³, R^{3′}, R⁴, R⁵, R^{5′}, R⁶, and L¹ is as defined and described herein, both individually and in combination.

In certain embodiments, the present invention provides a compound of formula I or I′ wherein X¹ is —CH₂—, X² is —CH₂—, and L² is

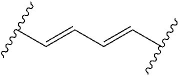 [SMILES: */C=C/C=C/*]

as shown below, thereby providing a compound of formula I-a-2:

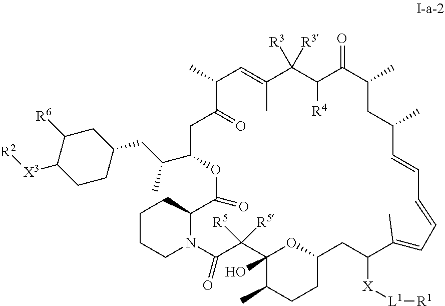

or a pharmaceutically acceptable salt thereof, wherein:
X³ is —CR₂—, —NRCO—, —NRCO₂—, —NRCONR—, —NRSO₂—, or —SO₂NR—; and each of X, R¹, R², R³, R^{3′}, R⁴, R⁵, R^{5′}, R⁶, and L¹ is as defined and described herein, both individually and in combination.

In certain embodiments, the present invention provides a compound of formula I or I′ wherein X¹ is —CH₂—, X² is —CH₂—, and L² is

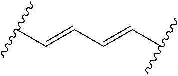 [SMILES: */C=C/C=C/*]

as shown below, thereby providing a compound of formula I-a-3:

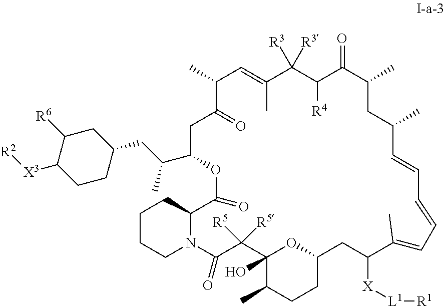

or a pharmaceutically acceptable salt thereof, wherein:
R⁴ is —NR₂, —NRCOR, —NRCO₂R, —NRCONR₂, —NRSO₂R, —SR, —SO₂NR₂, or an optionally substituted C₁₋₆ aliphatic; and
   each of X, X³, R¹, R², R³, R^{3′}, R⁵, R^{5′}, R⁶, and L¹ is as defined and described herein, both individually and in combination.

In certain embodiments, the present invention provides a compound of formula I or I′ wherein X¹ is —CH₂—, X² is —CH₂—, and L² is

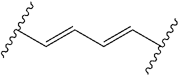 [SMILES: */C=C/C=C/*]

as shown below, thereby providing a compound of formula I-a-4:

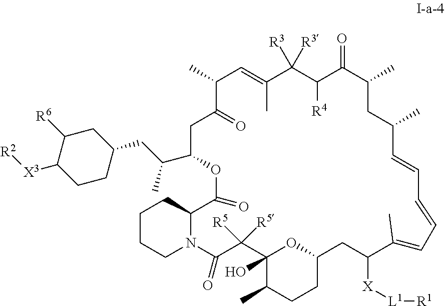

or a pharmaceutically acceptable salt thereof, wherein:
R⁶ is —NR₂, —NRCOR, —NRCO₂R, —NRCONR₂, —NRSO₂R, —SR, —SO₂NR₂, or an optionally substituted C₁₋₆ aliphatic; and
   each of X, X³, R¹, R², R³, R^{3′}, R⁴, R⁵, R^{5′}, and L¹ is as defined and described herein, both individually and in combination.

In certain embodiments, the present invention provides a compound of formula I or I′ wherein X¹ is —CH₂—, X² is —CH₂—, L² is

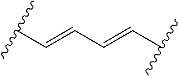 [SMILES: */C=C/C=C/*]

X³ is a covalent bond, and R² is hydrogen as shown below, thereby providing a compound of formula I-b-1:

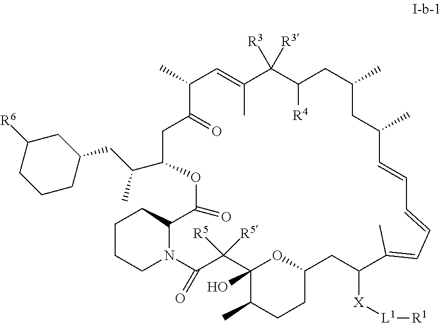

or a pharmaceutically acceptable salt thereof, wherein:
each of X, R¹, R³, R^{3′}, R⁴, R⁵, R^{5′}, R⁶, and L¹ is as defined and described herein, both individually and in combination.

In certain embodiments, the present invention provides a compound of formula I or I′ wherein X¹ is —CH₂—, X² is —CH₂—, L² is

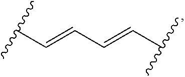 [SMILES: */C=C/C=C/*]

R³ is —OMe, and R^{3′} is hydrogen as shown below, thereby providing a compound of formula I-b-2:

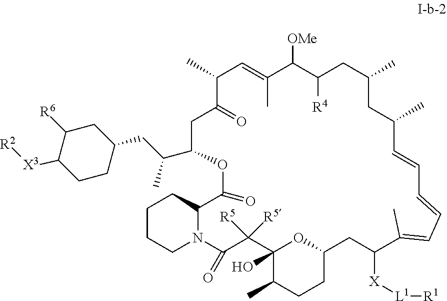

or a pharmaceutically acceptable salt thereof, wherein:
each of X, X³, R¹, R², R⁴, R⁵, R^{5′}, R⁶, and L¹ is as defined and described herein, both individually and in combination.

In certain embodiments, the present invention provides a compound of formula I or I′ wherein X¹ is —CH₂—, X² is —CH₂—, L² is

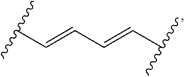 [SMILES: */C=C/C=C/*]

X³ is —O—, and R² is

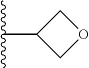 [SMILES: *C1COC1]

as shown below, thereby providing a compound of formula I-b-3:

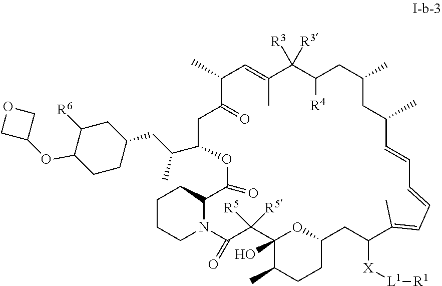

or a pharmaceutically acceptable salt thereof, wherein:
each of X, R¹, R³, R^{3′}, R⁴, R⁵, R^{5′}, R⁶, and L¹ is as defined and described herein, both individually and in combination.

Rapamycin is marketed under the brand name Rapamune® (generic name, sirolimus) and is well known for its antiproliferative and immunosuppressive activity. Rapamycin is FDA approved for the prevention of transplant rejection and for coating stents to prevent restenosis. Aside from the documented benefits of rapamycin, it is well known that rapamycin is associated with a number of serious side effects. Such side effects include diabetes-like symptoms of decreased glucose tolerance and lowering of insulin sensitivity. In addition, it has been reported that rapamycin activates the Akt signaling pathway (including activation of Akt and ERK) thereby increasing a patient's risk of cancer.

As used herein the phrase “rapamycin alone” is intended to compare a compound of the present invention with rapamycin, or an analog thereof such as everolimus, as alternatives.

In some embodiments, a provided compound of formula I or I′ is more efficacious than rapamycin alone.

In some embodiments, a provided compound of formula I-a-1 to I-a-5 is more efficacious than rapamycin alone.

In some embodiments, a provided compound of formula I-b-1 to I-b-3 is more efficacious than rapamycin alone.

In some embodiments, a provided compound of formula I or I′, when administered to a patient, results in fewer and/or lesser severity of side effects than when rapamycin is administered.

In some embodiments, a provided compound of formula I-a-1 to I-a-5, when administered to a patient, results in fewer and/or lesser severity of side effects than when rapamycin is administered.

In some embodiments, a provided compound of formula I-b-1 to I-b-3, when administered to a patient, results in fewer and/or lesser severity of side effects than when rapamycin is administered.

Exemplary compounds of the invention are set forth in Table 1, below.

TABLE 1
Exemplary Compounds

| I-# | Structure |
|---|---|
| I-1 | 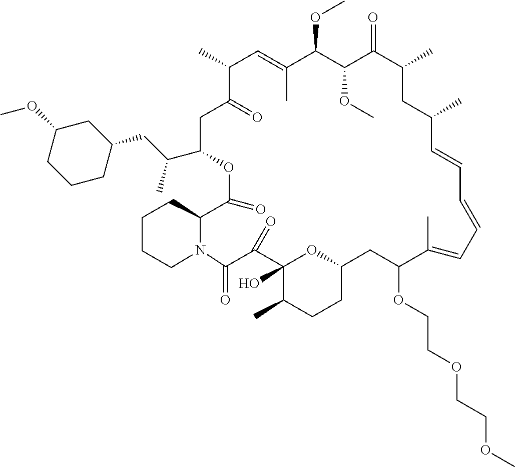 |
| I-2 | 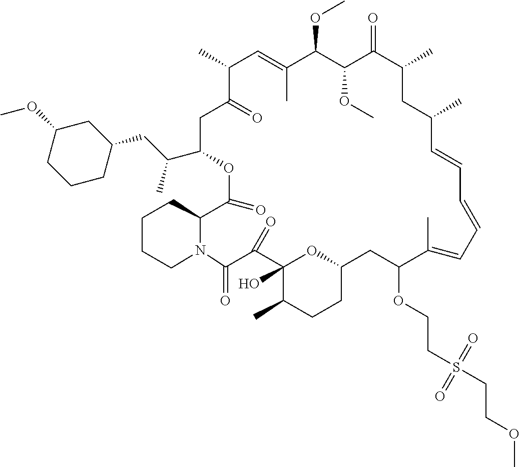 |
| I-3 | 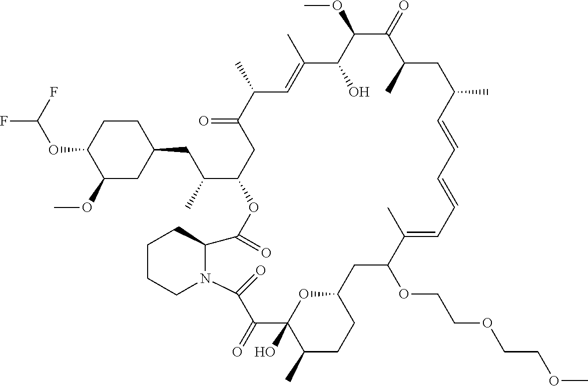 [SMILES: COCCOCCOC1C[C@@H]2CC[C@@H](C)[C@@](O)(O2)C(=O)C(=O)N2CCCC[C@H]2C(=O)O[C@H]([C@H](C)C[C@@H]2CC[C@@H](OC(F)F)[C@H](OC)C2)CC(=O)[C@H](C)/C=C(\C)[C@@H](O)[C@@H](OC)C(=O)[C@H](C)C[C@H](C)/C=C/C=C/C=C/1C] |
| I-4 | 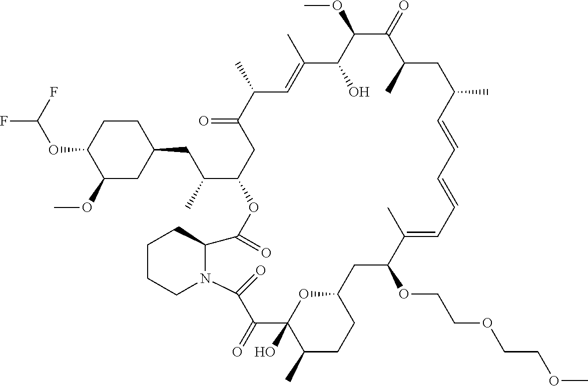 [SMILES: COCCOCCO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(F)F)[C@H](OC)C2)CC(=O)[C@H](C)/C=C(\C)[C@@H](O)[C@@H](OC)C(=O)[C@H](C)C[C@H](C)/C=C/C=C/C=C/1C] |
| I-5 | 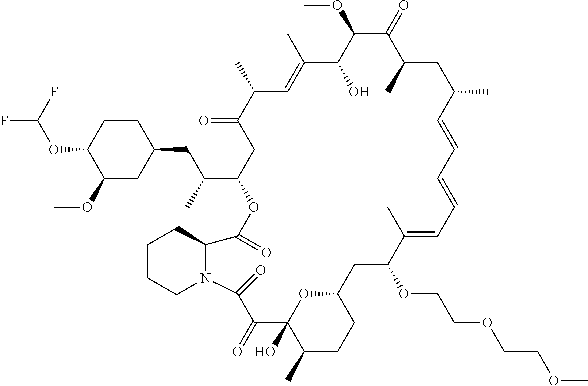 [SMILES: COCCOCCO[C@@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(F)F)[C@H](OC)C2)CC(=O)[C@H](C)/C=C(\C)[C@@H](O)[C@@H](OC)C(=O)[C@H](C)C[C@H](C)/C=C/C=C/C=C/1C] |
| I-6 | 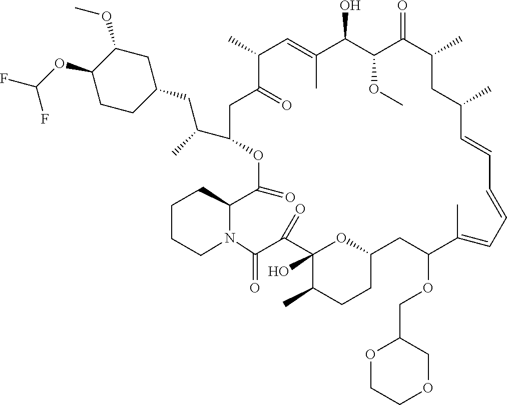 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCC3COCCO3)C[C@@H]3CC[C@@H](C)[C@@](O)(O3)C(=O)C(=O)N3CCCC[C@H]3C(=O)O2)CC[C@H]1OC(F)F] |
| I-7 | 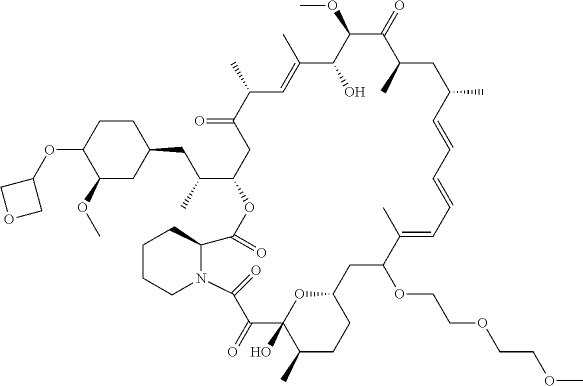 [SMILES: COCCOCCOC1C[C@@H]2CC[C@@H](C)[C@@](O)(O2)C(=O)C(=O)N2CCCC[C@H]2C(=O)O[C@H]([C@H](C)C[C@@H]2CCC(OC3COC3)[C@H](OC)C2)CC(=O)[C@H](C)/C=C(\C)[C@@H](O)[C@@H](OC)C(=O)[C@H](C)C[C@H](C)/C=C/C=C/C=C/1C] |
| I-8 | 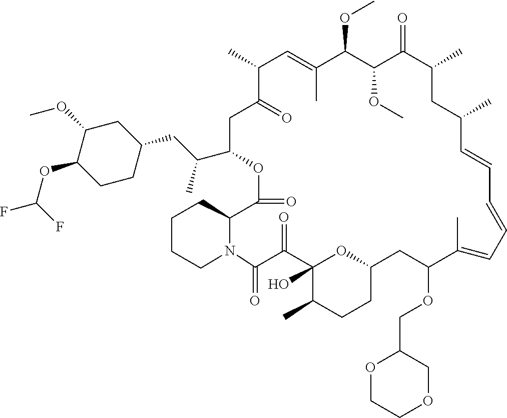 [SMILES: CO[C@@H]1/C(C)=C/[C@@H](C)C(=O)C[C@@H]([C@H](C)C[C@@H]2CC[C@@H](OC(F)F)[C@H](OC)C2)OC(=O)[C@@H]2CCCCN2C(=O)C(=O)[C@]2(O)O[C@@H](CC[C@H]2C)CC(OCC2COCCO2)/C(C)=C/C=C/C=C/[C@@H](C)C[C@@H](C)C(=O)[C@@H]1OC] |
| I-9 | 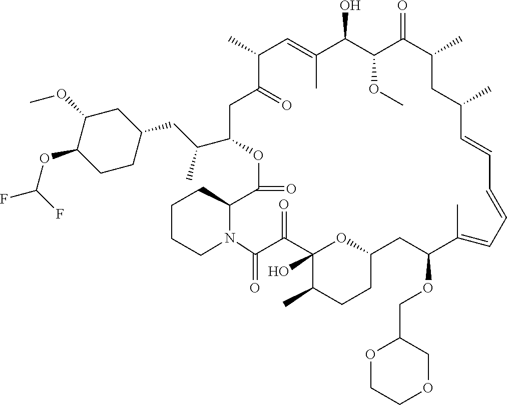 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OCC3COCCO3)C[C@@H]3CC[C@@H](C)[C@@](O)(O3)C(=O)C(=O)N3CCCC[C@H]3C(=O)O2)CC[C@H]1OC(F)F] |
| I-10 | 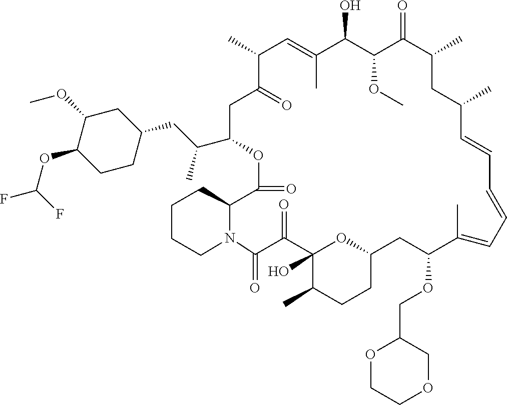 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OCC3COCCO3)C[C@@H]3CC[C@@H](C)[C@@](O)(O3)C(=O)C(=O)N3CCCC[C@H]3C(=O)O2)CC[C@H]1OC(F)F] |
| I-11 | 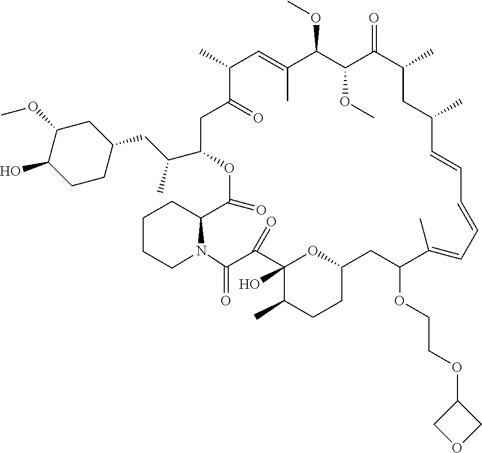 [SMILES: CO[C@@H]1/C(C)=C/[C@@H](C)C(=O)C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)CC(OCCOC2COC2)/C(C)=C/C=C/C=C/[C@@H](C)C[C@@H](C)C(=O)[C@@H]1OC] |
| I-12 | 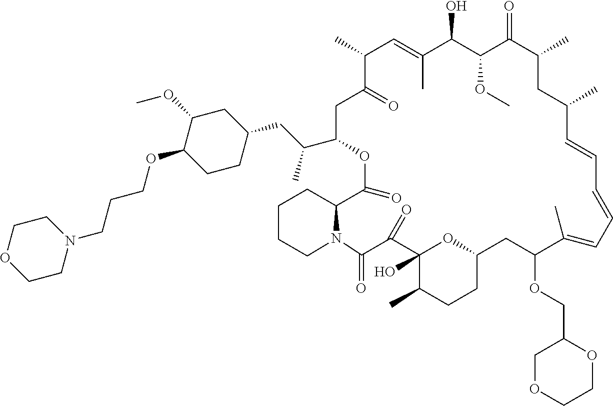 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCC3COCCO3)C[C@@H]3CC[C@@H](C)[C@@](O)(O3)C(=O)C(=O)N3CCCC[C@H]3C(=O)O2)CC[C@H]1OCCCN1CCOCC1] |
| I-13 | 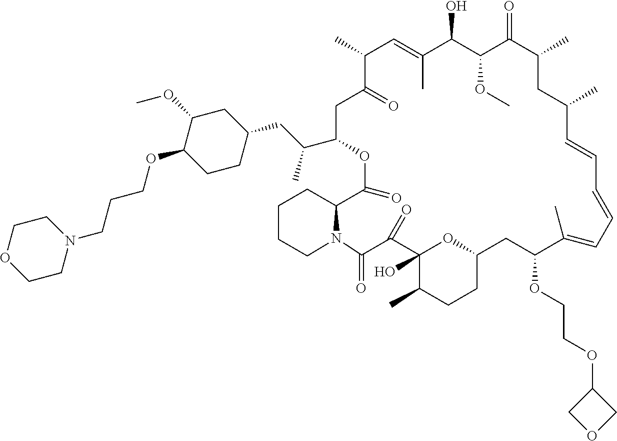 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OCCOC3COC3)C[C@@H]3CC[C@@H](C)[C@@](O)(O3)C(=O)C(=O)N3CCCC[C@H]3C(=O)O2)CC[C@H]1OCCCN1CCOCC1] |
| I-14 | 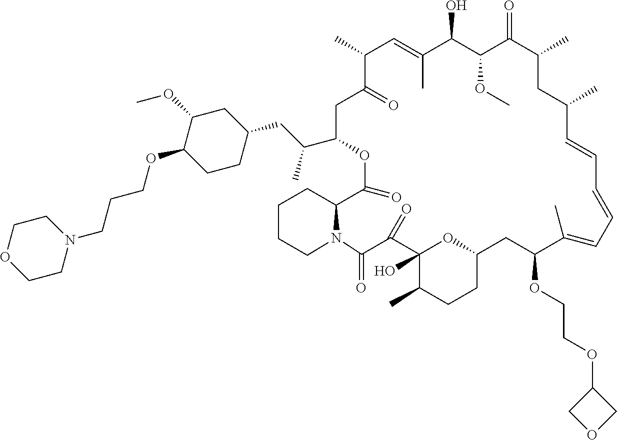 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OCCOC3COC3)C[C@@H]3CC[C@@H](C)[C@@](O)(O3)C(=O)C(=O)N3CCCC[C@H]3C(=O)O2)CC[C@H]1OCCCN1CCOCC1] |
| I-15 | 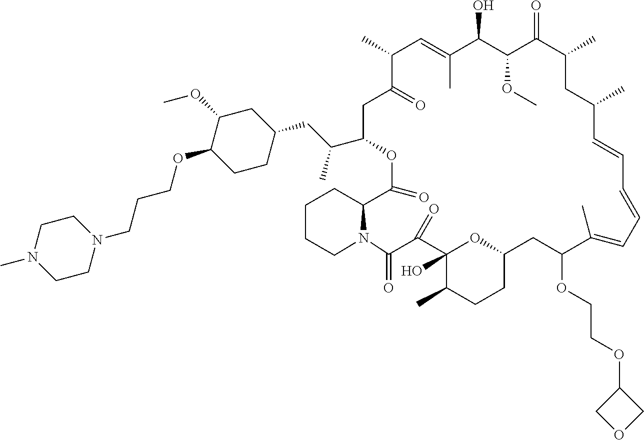 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCCOC3COC3)C[C@@H]3CC[C@@H](C)[C@@](O)(O3)C(=O)C(=O)N3CCCC[C@H]3C(=O)O2)CC[C@H]1OCCCN1CCN(C)CC1] |
| I-16 | 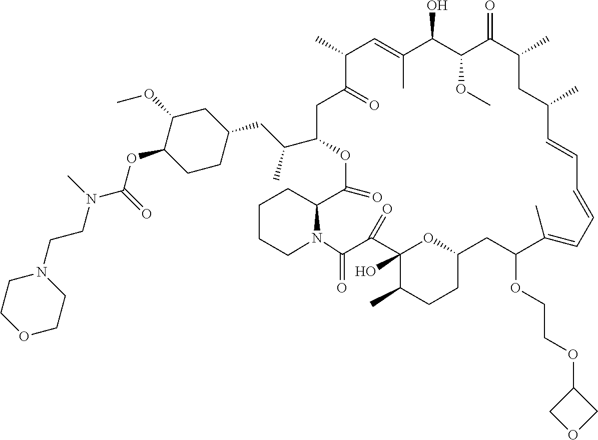 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCCOC3COC3)C[C@@H]3CC[C@@H](C)[C@@](O)(O3)C(=O)C(=O)N3CCCC[C@H]3C(=O)O2)CC[C@H]1OC(=O)N(C)CCN1CCOCC1] |
| I-17 | 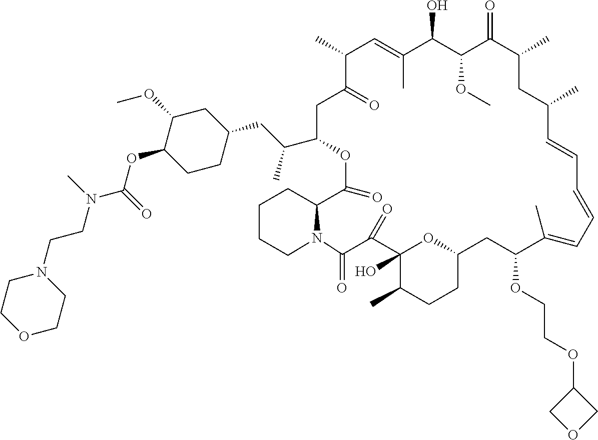 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OCCOC3COC3)C[C@@H]3CC[C@@H](C)[C@@](O)(O3)C(=O)C(=O)N3CCCC[C@H]3C(=O)O2)CC[C@H]1OC(=O)N(C)CCN1CCOCC1] |
| I-18 | 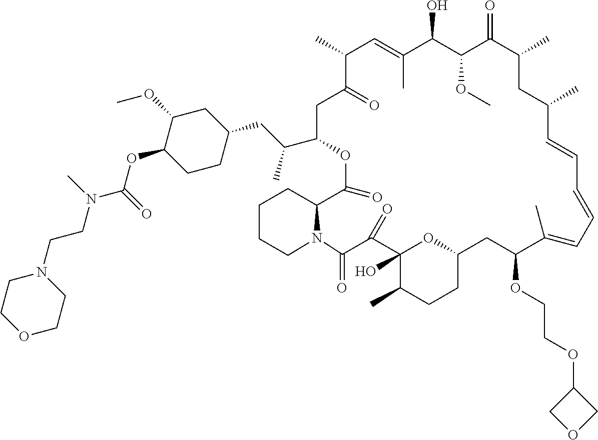 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OCCOC3COC3)C[C@@H]3CC[C@@H](C)[C@@](O)(O3)C(=O)C(=O)N3CCCC[C@H]3C(=O)O2)CC[C@H]1OC(=O)N(C)CCN1CCOCC1] |
| I-19 | 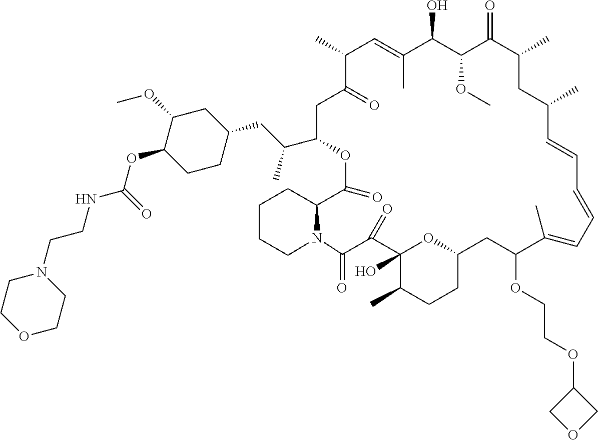 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCCOC3COC3)C[C@@H]3CC[C@@H](C)[C@@](O)(O3)C(=O)C(=O)N3CCCC[C@H]3C(=O)O2)CC[C@H]1OC(=O)NCCN1CCOCC1] |
| I-20 | 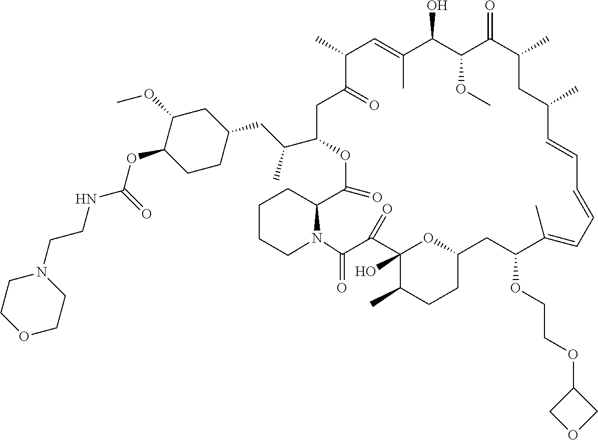 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OCCOC3COC3)C[C@@H]3CC[C@@H](C)[C@@](O)(O3)C(=O)C(=O)N3CCCC[C@H]3C(=O)O2)CC[C@H]1OC(=O)NCCN1CCOCC1] |
| I-21 | 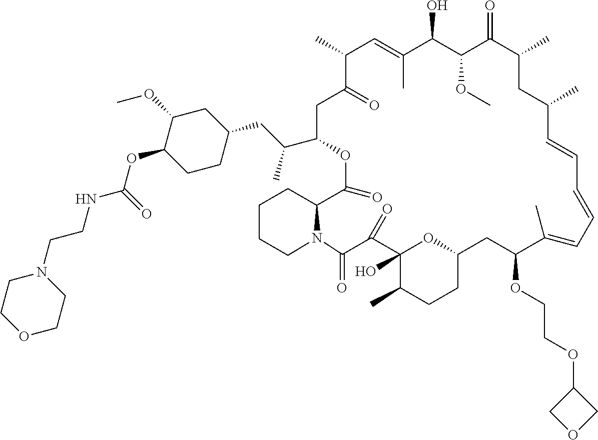 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OCCOC3COC3)C[C@@H]3CC[C@@H](C)[C@@](O)(O3)C(=O)C(=O)N3CCCC[C@H]3C(=O)O2)CC[C@H]1OC(=O)NCCN1CCOCC1] |
| I-22 | 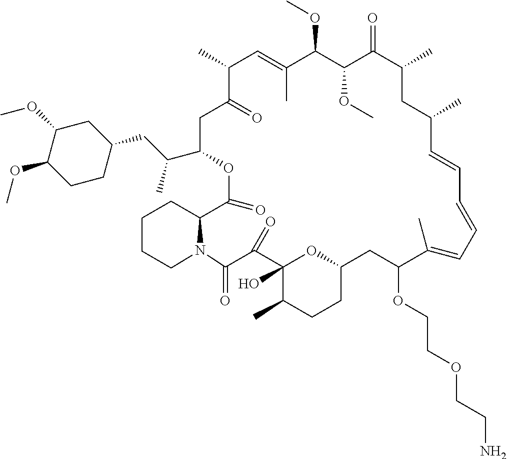 [SMILES: CO[C@@H]1/C(C)=C/[C@@H](C)C(=O)C[C@@H]([C@H](C)C[C@@H]2CC[C@@H](OC)[C@H](OC)C2)OC(=O)[C@@H]2CCCCN2C(=O)C(=O)[C@]2(O)O[C@@H](CC[C@H]2C)CC(OCCOCCN)/C(C)=C/C=C/C=C/[C@@H](C)C[C@@H](C)C(=O)[C@@H]1OC] |
| I-23 | 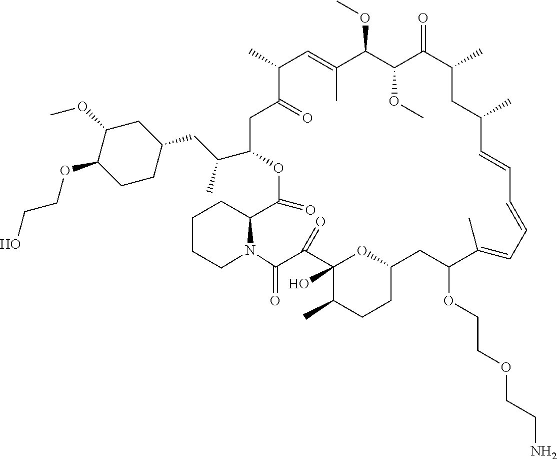 [SMILES: CO[C@@H]1/C(C)=C/[C@@H](C)C(=O)C[C@@H]([C@H](C)C[C@@H]2CC[C@@H](OCCO)[C@H](OC)C2)OC(=O)[C@@H]2CCCCN2C(=O)C(=O)[C@]2(O)O[C@@H](CC[C@H]2C)CC(OCCOCCN)/C(C)=C/C=C/C=C/[C@@H](C)C[C@@H](C)C(=O)[C@@H]1OC] |
| I-24 | 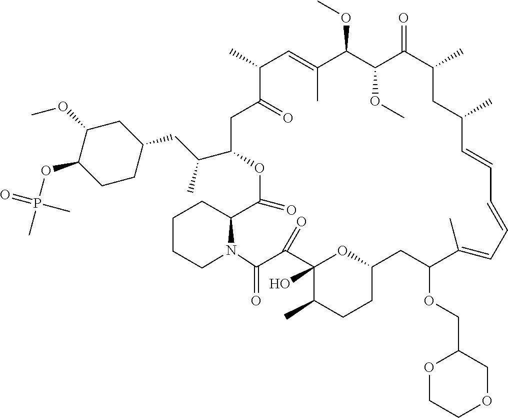 [SMILES: CO[C@@H]1/C(C)=C/[C@@H](C)C(=O)C[C@@H]([C@H](C)C[C@@H]2CC[C@@H](OP(C)(C)=O)[C@H](OC)C2)OC(=O)[C@@H]2CCCCN2C(=O)C(=O)[C@]2(O)O[C@@H](CC[C@H]2C)CC(OCC2COCCO2)/C(C)=C/C=C/C=C/[C@@H](C)C[C@@H](C)C(=O)[C@@H]1OC] |
| I-25 | 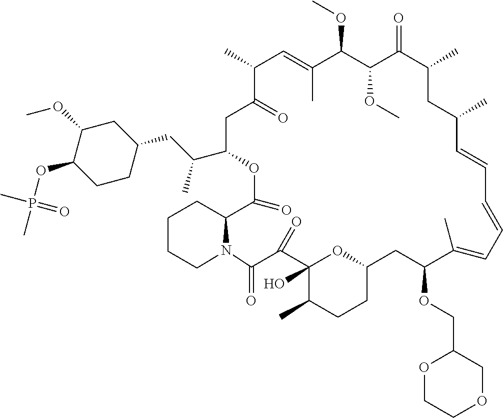 [SMILES: CO[C@@H]1/C(C)=C/[C@@H](C)C(=O)C[C@@H]([C@H](C)C[C@@H]2CC[C@@H](OP(C)(C)=O)[C@H](OC)C2)OC(=O)[C@@H]2CCCCN2C(=O)C(=O)[C@]2(O)O[C@@H](CC[C@H]2C)C[C@H](OCC2COCCO2)/C(C)=C/C=C/C=C/[C@@H](C)C[C@@H](C)C(=O)[C@@H]1OC] |
| I-26 | 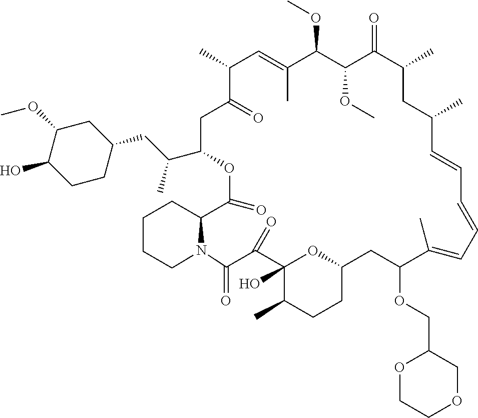 [SMILES: CO[C@@H]1/C(C)=C/[C@@H](C)C(=O)C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)CC(OCC2COCCO2)/C(C)=C/C=C/C=C/[C@@H](C)C[C@@H](C)C(=O)[C@@H]1OC] |
| I-27 | 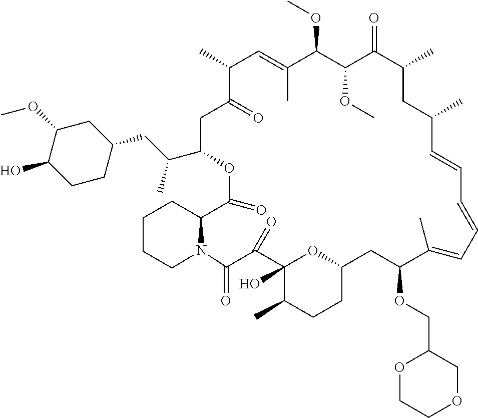 [SMILES: CO[C@@H]1/C(C)=C/[C@@H](C)C(=O)C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)C[C@H](OCC2COCCO2)/C(C)=C/C=C/C=C/[C@@H](C)C[C@@H](C)C(=O)[C@@H]1OC] |
| I-28 | 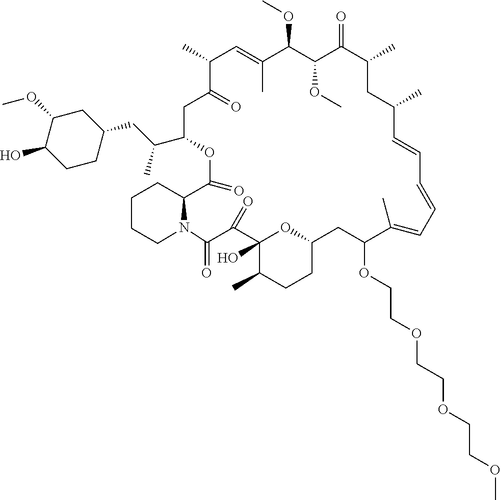 |
| I-29 | 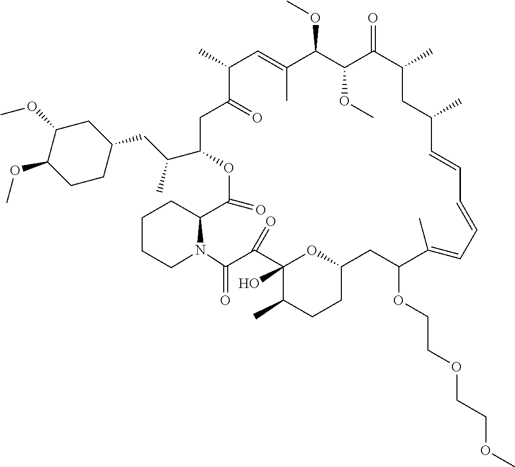 |
| I-30 | 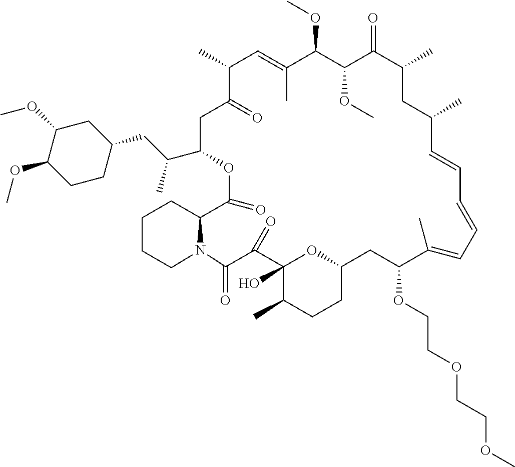 |
| I-31 | 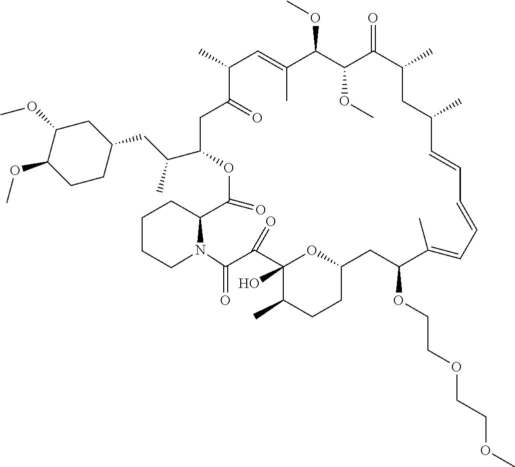 |
| I-32 | 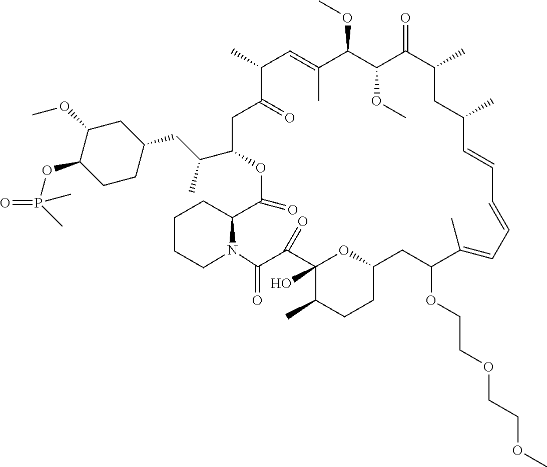 [SMILES: COCCOCCOC1C[C@@H]2CC[C@@H](C)[C@@](O)(O2)C(=O)C(=O)N2CCCC[C@H]2C(=O)O[C@H]([C@H](C)C[C@@H]2CC[C@@H](OP(C)(C)=O)[C@H](OC)C2)CC(=O)[C@H](C)/C=C(\C)[C@@H](OC)[C@@H](OC)C(=O)[C@H](C)C[C@H](C)/C=C/C=C/C=C/1C] |
| I-33 | 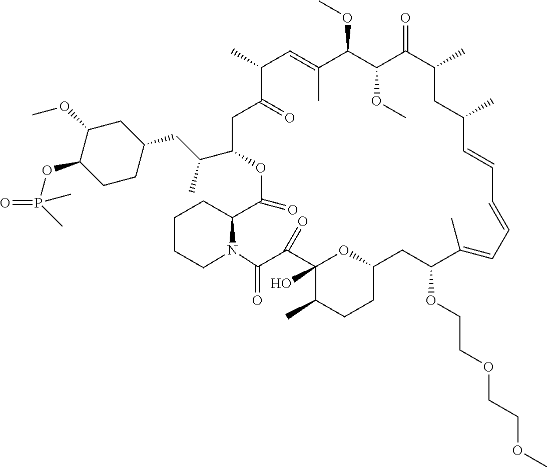 [SMILES: COCCOCCO[C@@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OP(C)(C)=O)[C@H](OC)C2)CC(=O)[C@H](C)/C=C(\C)[C@@H](OC)[C@@H](OC)C(=O)[C@H](C)C[C@H](C)/C=C/C=C/C=C/1C] |
| I-34 | 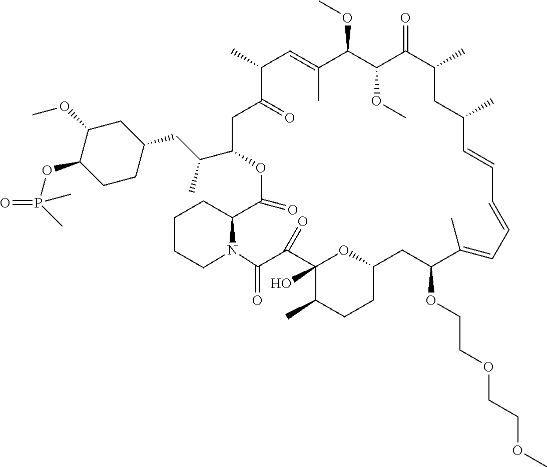 [SMILES: COCCOCCO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OP(C)(C)=O)[C@H](OC)C2)CC(=O)[C@H](C)/C=C(\C)[C@@H](OC)[C@@H](OC)C(=O)[C@H](C)C[C@H](C)/C=C/C=C/C=C/1C] |
| I-35 | 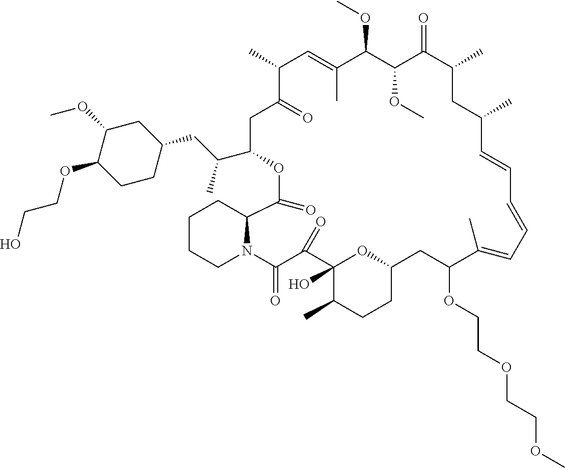 [SMILES: COCCOCCOC1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](OC)[C@@H](OC)C(=O)[C@H](C)C[C@H](C)/C=C/C=C/C=C/1C] |
| I-36 | 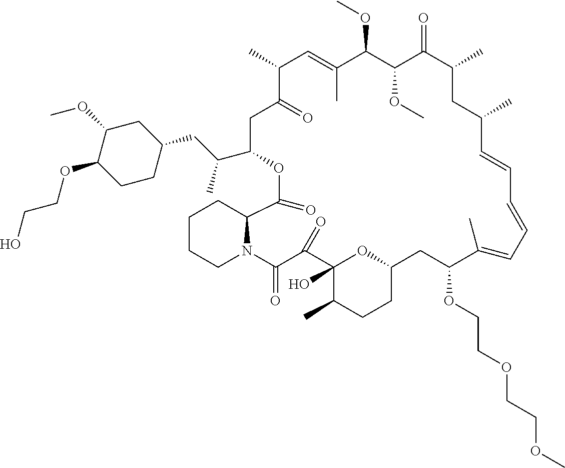 [SMILES: COCCOCCO[C@@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](OC)[C@@H](OC)C(=O)[C@H](C)C[C@H](C)/C=C/C=C/C=C/1C] |
| I-37 | 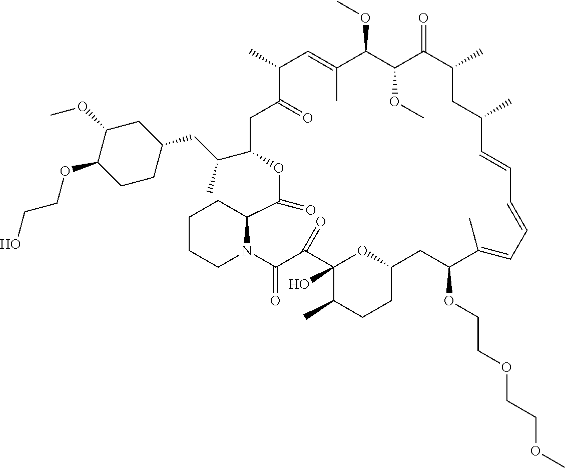 [SMILES: COCCOCCO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](OC)[C@@H](OC)C(=O)[C@H](C)C[C@H](C)/C=C/C=C/C=C/1C] |
| I-38 | 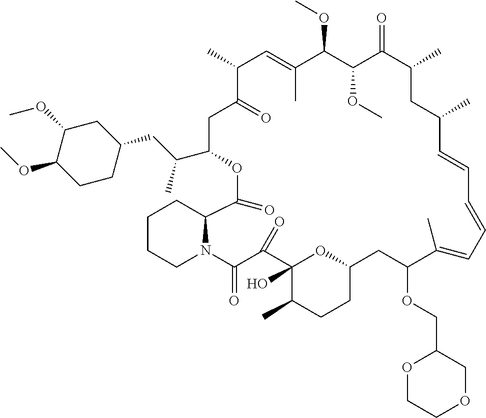 [SMILES: CO[C@@H]1/C(C)=C/[C@@H](C)C(=O)C[C@@H]([C@H](C)C[C@@H]2CC[C@@H](OC)[C@H](OC)C2)OC(=O)[C@@H]2CCCCN2C(=O)C(=O)[C@]2(O)O[C@@H](CC[C@H]2C)CC(OCC2COCCO2)/C(C)=C/C=C/C=C/[C@@H](C)C[C@@H](C)C(=O)[C@@H]1OC] |
| I-39 | 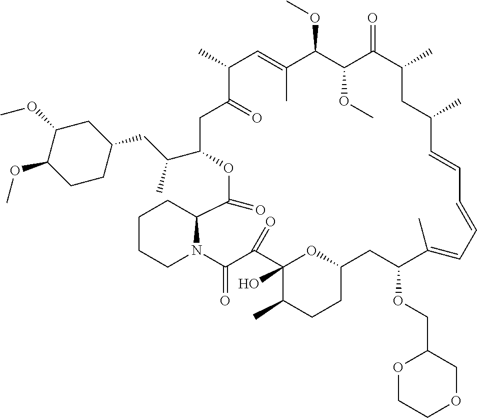 [SMILES: CO[C@@H]1/C(C)=C/[C@@H](C)C(=O)C[C@@H]([C@H](C)C[C@@H]2CC[C@@H](OC)[C@H](OC)C2)OC(=O)[C@@H]2CCCCN2C(=O)C(=O)[C@]2(O)O[C@@H](CC[C@H]2C)C[C@@H](OCC2COCCO2)/C(C)=C/C=C/C=C/[C@@H](C)C[C@@H](C)C(=O)[C@@H]1OC] |
| I-40 | 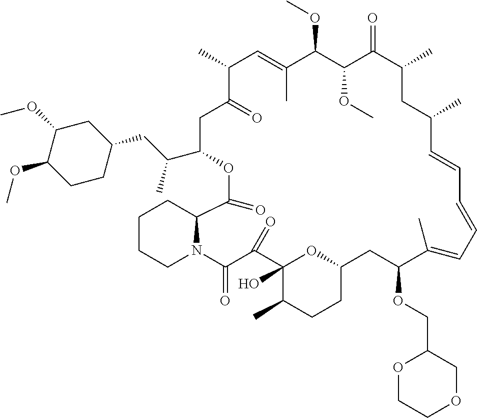 [SMILES: CO[C@@H]1/C(C)=C/[C@@H](C)C(=O)C[C@@H]([C@H](C)C[C@@H]2CC[C@@H](OC)[C@H](OC)C2)OC(=O)[C@@H]2CCCCN2C(=O)C(=O)[C@]2(O)O[C@@H](CC[C@H]2C)C[C@H](OCC2COCCO2)/C(C)=C/C=C/C=C/[C@@H](C)C[C@@H](C)C(=O)[C@@H]1OC] |
| I-41 | 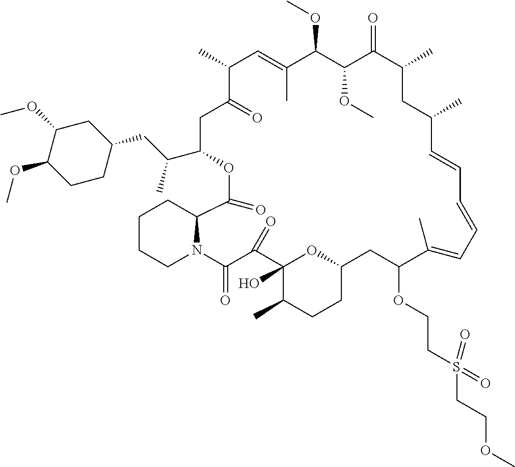 |
| I-42 | 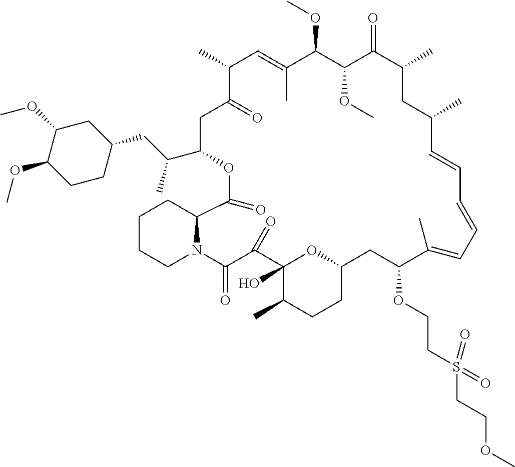 |
| I-43 | 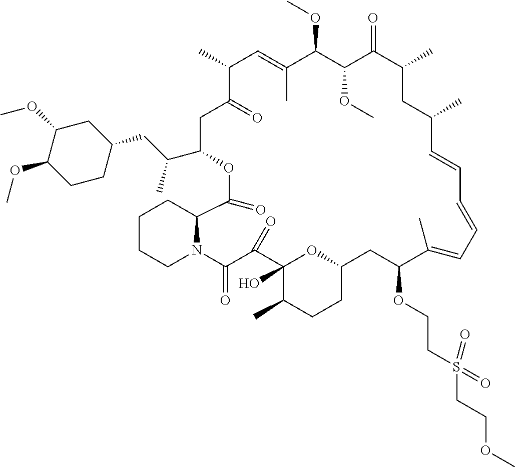 |
| I-44 | 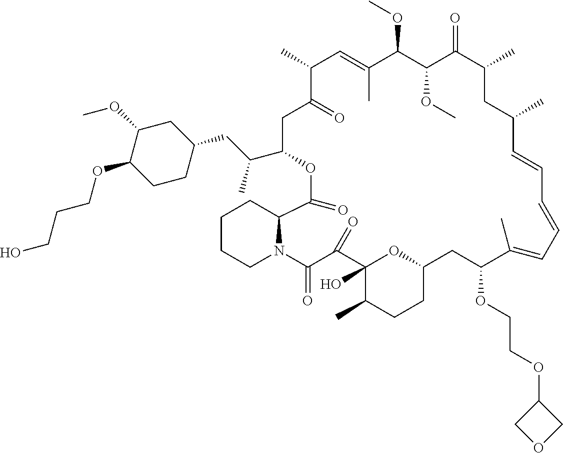 [SMILES: CO[C@@H]1/C(C)=C/[C@@H](C)C(=O)C[C@@H]([C@H](C)C[C@@H]2CC[C@@H](OCCCO)[C@H](OC)C2)OC(=O)[C@@H]2CCCCN2C(=O)C(=O)[C@]2(O)O[C@@H](CC[C@H]2C)C[C@@H](OCCOC2COC2)/C(C)=C/C=C/C=C/[C@@H](C)C[C@@H](C)C(=O)[C@@H]1OC] |
| I-45 | 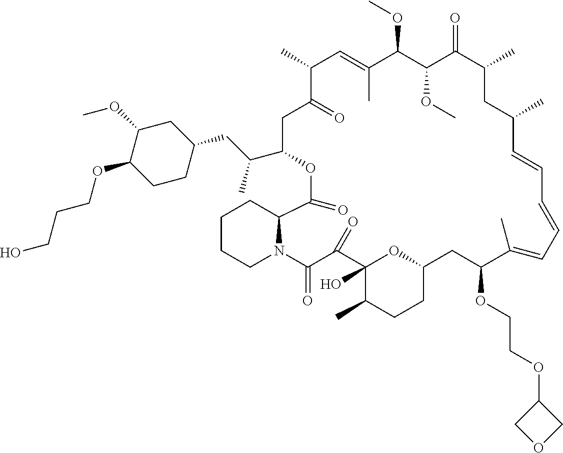 [SMILES: CO[C@@H]1/C(C)=C/[C@@H](C)C(=O)C[C@@H]([C@H](C)C[C@@H]2CC[C@@H](OCCCO)[C@H](OC)C2)OC(=O)[C@@H]2CCCCN2C(=O)C(=O)[C@]2(O)O[C@@H](CC[C@H]2C)C[C@H](OCCOC2COC2)/C(C)=C/C=C/C=C/[C@@H](C)C[C@@H](C)C(=O)[C@@H]1OC] |
| I-46 | 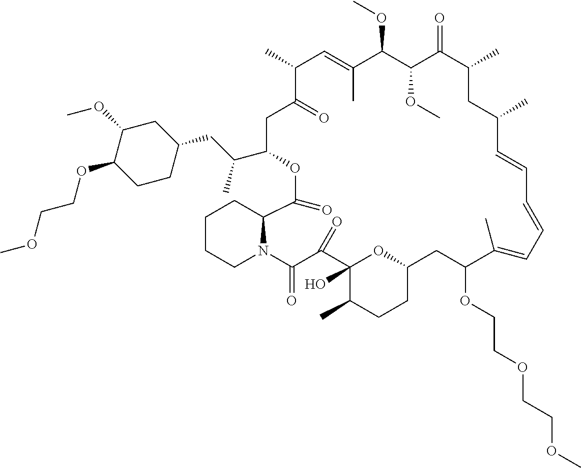 |
| I-47 | 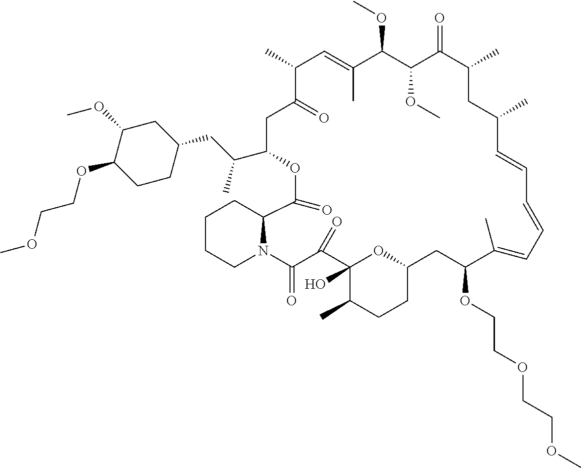 |
| I-48 | 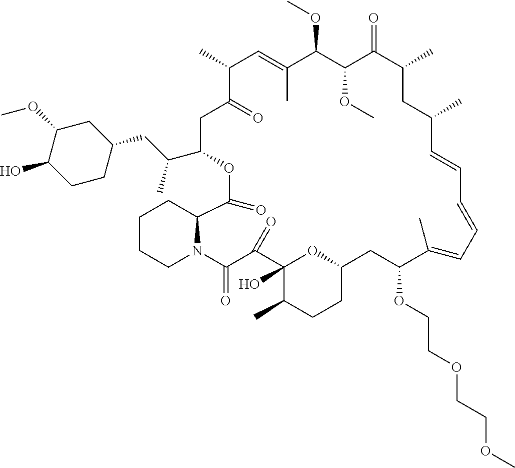 |
| I-49 | 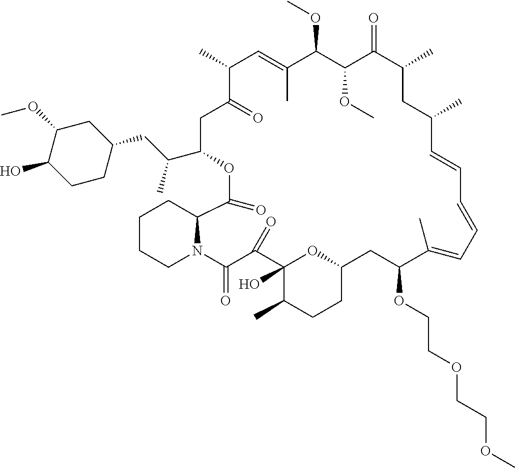 |
| I-50 | 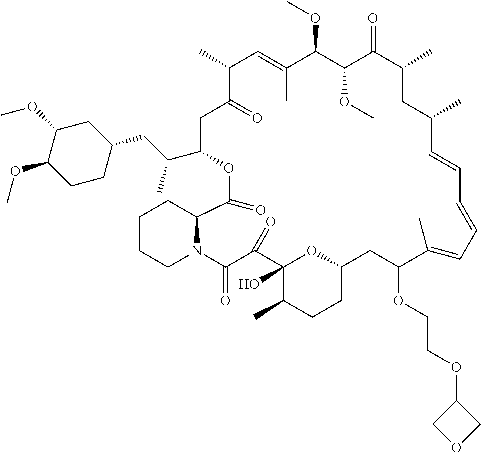 [SMILES: CO[C@@H]1/C(C)=C/[C@@H](C)C(=O)C[C@@H]([C@H](C)C[C@@H]2CC[C@@H](OC)[C@H](OC)C2)OC(=O)[C@@H]2CCCCN2C(=O)C(=O)[C@]2(O)O[C@@H](CC[C@H]2C)CC(OCCOC2COC2)/C(C)=C/C=C/C=C/[C@@H](C)C[C@@H](C)C(=O)[C@@H]1OC] |
| I-51 | 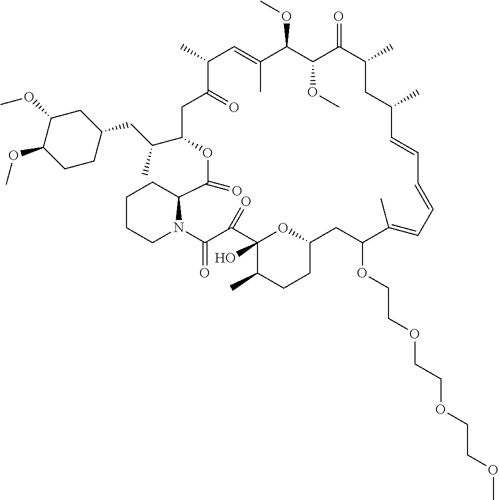 |
| I-52 | 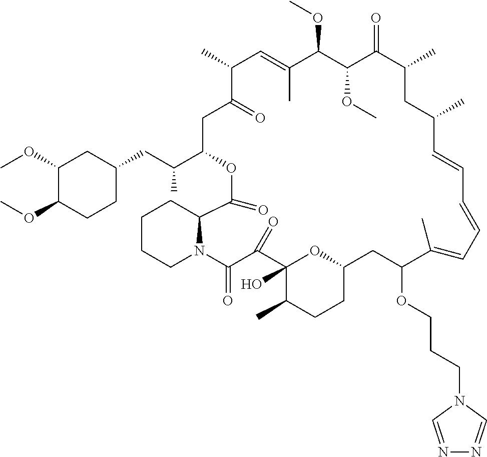 [SMILES: CO[C@@H]1/C(C)=C/[C@@H](C)C(=O)C[C@@H]([C@H](C)C[C@@H]2CC[C@@H](OC)[C@H](OC)C2)OC(=O)[C@@H]2CCCCN2C(=O)C(=O)[C@]2(O)O[C@@H](CC[C@H]2C)CC(OCCCn2cnnc2)/C(C)=C/C=C/C=C/[C@@H](C)C[C@@H](C)C(=O)[C@@H]1OC] |
| I-53 | 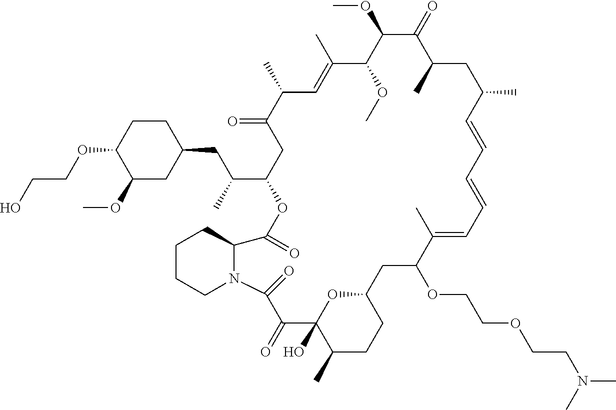 [SMILES: CO[C@@H]1/C(C)=C/[C@@H](C)C(=O)C[C@@H]([C@H](C)C[C@@H]2CC[C@@H](OCCO)[C@H](OC)C2)OC(=O)[C@@H]2CCCCN2C(=O)C(=O)[C@]2(O)O[C@@H](CC[C@H]2C)CC(OCCOCCN(C)C)/C(C)=C/C=C/C=C/[C@@H](C)C[C@@H](C)C(=O)[C@@H]1OC] |
| I-54 | 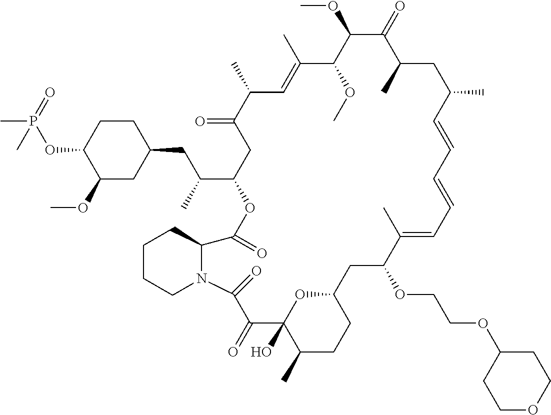 [SMILES: CO[C@@H]1/C(C)=C/[C@@H](C)C(=O)C[C@@H]([C@H](C)C[C@@H]2CC[C@@H](OP(C)(C)=O)[C@H](OC)C2)OC(=O)[C@@H]2CCCCN2C(=O)C(=O)[C@]2(O)O[C@@H](CC[C@H]2C)C[C@@H](OCCOC2CCOCC2)/C(C)=C/C=C/C=C/[C@@H](C)C[C@@H](C)C(=O)[C@@H]1OC] |
| I-55 | 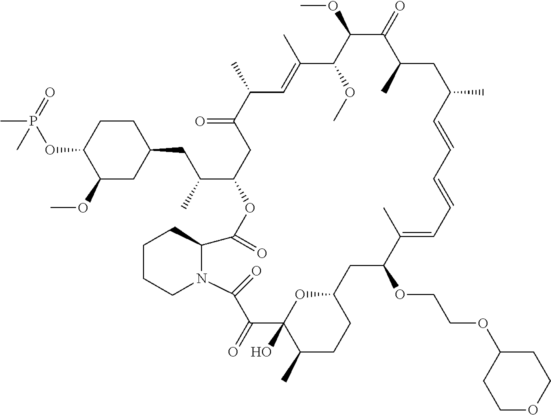 [SMILES: CO[C@@H]1/C(C)=C/[C@@H](C)C(=O)C[C@@H]([C@H](C)C[C@@H]2CC[C@@H](OP(C)(C)=O)[C@H](OC)C2)OC(=O)[C@@H]2CCCCN2C(=O)C(=O)[C@]2(O)O[C@@H](CC[C@H]2C)C[C@H](OCCOC2CCOCC2)/C(C)=C/C=C/C=C/[C@@H](C)C[C@@H](C)C(=O)[C@@H]1OC] |
| I-56 | 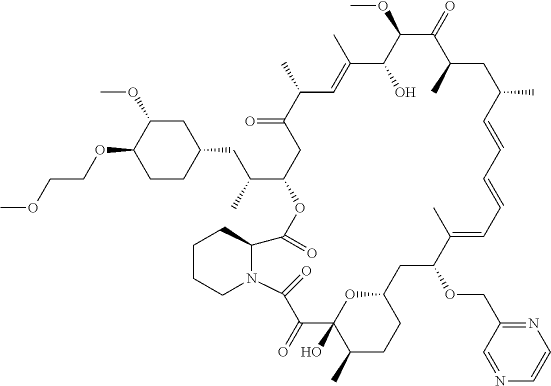 [SMILES: COCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OCc3cnccn3)C[C@@H]3CC[C@@H](C)[C@@](O)(O3)C(=O)C(=O)N3CCCC[C@H]3C(=O)O2)C[C@H]1OC] |
| I-57 | 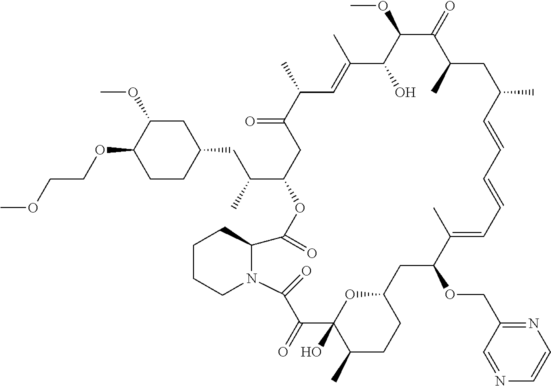 [SMILES: COCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OCc3cnccn3)C[C@@H]3CC[C@@H](C)[C@@](O)(O3)C(=O)C(=O)N3CCCC[C@H]3C(=O)O2)C[C@H]1OC] |
| I-58 | 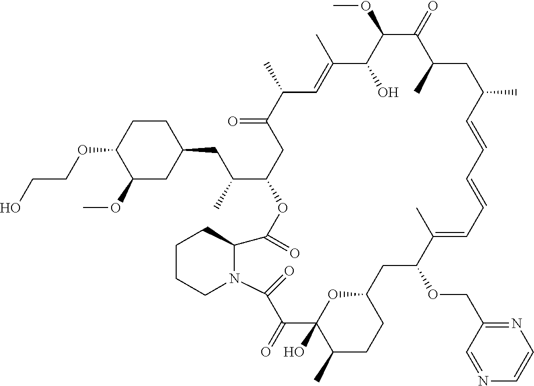 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OCc3cnccn3)C[C@@H]3CC[C@@H](C)[C@@](O)(O3)C(=O)C(=O)N3CCCC[C@H]3C(=O)O2)CC[C@H]1OCCO] |
| I-59 | 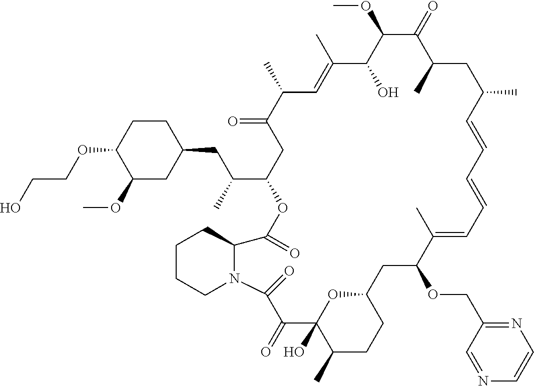 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OCc3cnccn3)C[C@@H]3CC[C@@H](C)[C@@](O)(O3)C(=O)C(=O)N3CCCC[C@H]3C(=O)O2)CC[C@H]1OCCO] |
| I-60 | 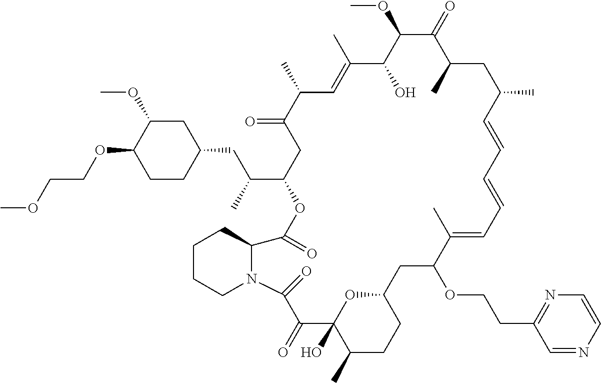 [SMILES: COCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCCc3cnccn3)C[C@@H]3CC[C@@H](C)[C@@](O)(O3)C(=O)C(=O)N3CCCC[C@H]3C(=O)O2)C[C@H]1OC] |
| I-61 | 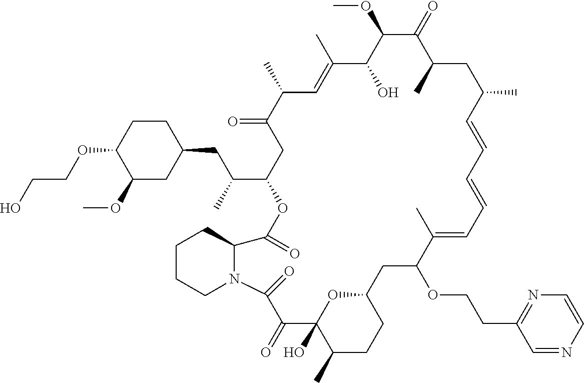 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCCc3cnccn3)C[C@@H]3CC[C@@H](C)[C@@](O)(O3)C(=O)C(=O)N3CCCC[C@H]3C(=O)O2)CC[C@H]1OCCO] |
| I-62 | 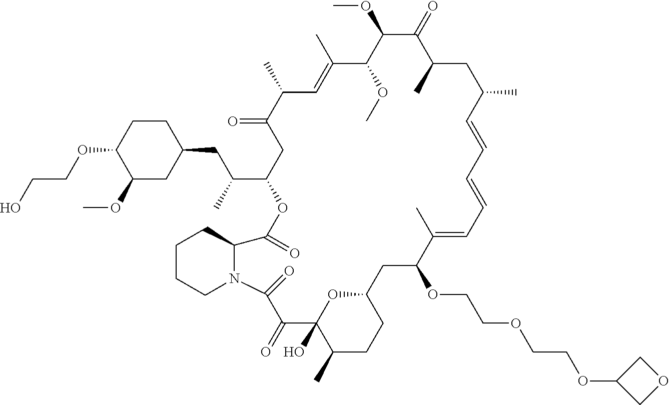 [SMILES: CO[C@@H]1/C(C)=C/[C@@H](C)C(=O)C[C@@H]([C@H](C)C[C@@H]2CC[C@@H](OCCO)[C@H](OC)C2)OC(=O)[C@@H]2CCCCN2C(=O)C(=O)[C@]2(O)O[C@@H](CC[C@H]2C)C[C@H](OCCOCCOC2COC2)/C(C)=C/C=C/C=C/[C@@H](C)C[C@@H](C)C(=O)[C@@H]1OC] |
| I-63 | 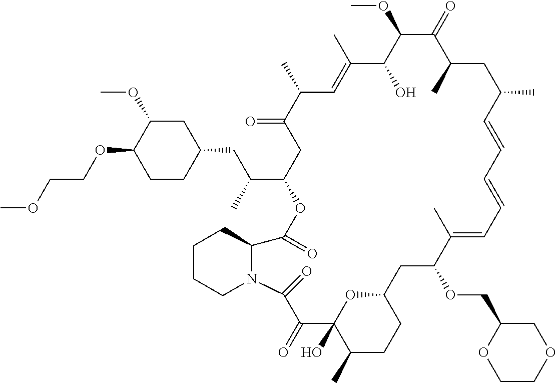 [SMILES: COCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OC[C@H]3COCCO3)C[C@@H]3CC[C@@H](C)[C@@](O)(O3)C(=O)C(=O)N3CCCC[C@H]3C(=O)O2)C[C@H]1OC] |
| I-64 | 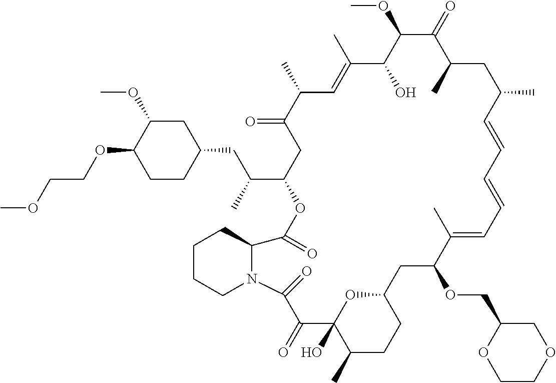 [SMILES: COCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC[C@H]3COCCO3)C[C@@H]3CC[C@@H](C)[C@@](O)(O3)C(=O)C(=O)N3CCCC[C@H]3C(=O)O2)C[C@H]1OC] |
| I-65 | 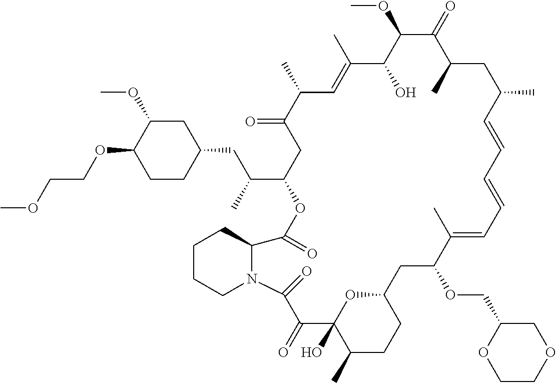 [SMILES: COCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OC[C@@H]3COCCO3)C[C@@H]3CC[C@@H](C)[C@@](O)(O3)C(=O)C(=O)N3CCCC[C@H]3C(=O)O2)C[C@H]1OC] |
| I-66 | 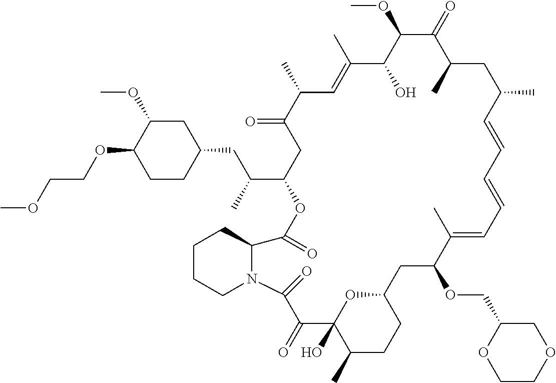 [SMILES: COCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC[C@@H]3COCCO3)C[C@@H]3CC[C@@H](C)[C@@](O)(O3)C(=O)C(=O)N3CCCC[C@H]3C(=O)O2)C[C@H]1OC] |
| I-67 | 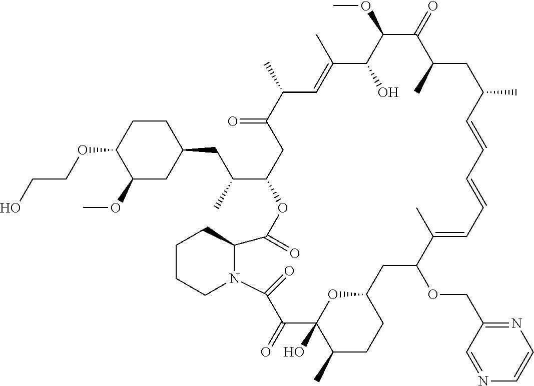 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCc3cnccn3)C[C@@H]3CC[C@@H](C)[C@@](O)(O3)C(=O)C(=O)N3CCCC[C@H]3C(=O)O2)CC[C@H]1OCCO] |
| I-68 | 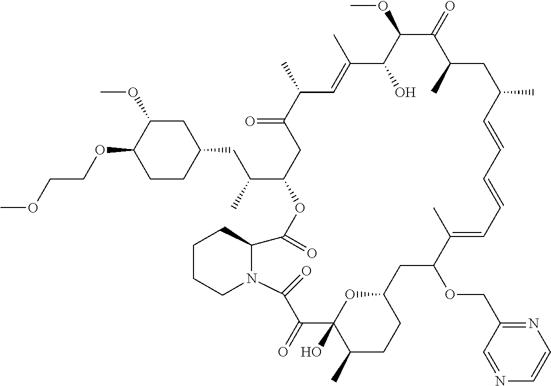 [SMILES: COCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCc3cnccn3)C[C@@H]3CC[C@@H](C)[C@@](O)(O3)C(=O)C(=O)N3CCCC[C@H]3C(=O)O2)C[C@H]1OC] |
| I-69 | 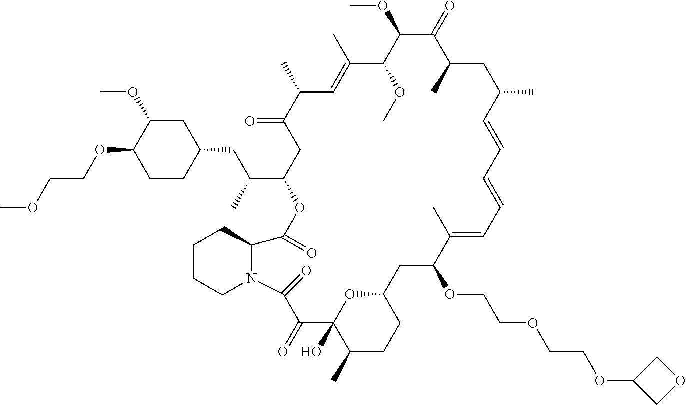 [SMILES: COCCO[C@@H]1CC[C@@H](C[C@@H](C)[C@@H]2CC(=O)[C@H](C)/C=C(\C)[C@@H](OC)[C@@H](OC)C(=O)[C@H](C)C[C@H](C)/C=C/C=C/C=C(\C)[C@@H](OCCOCCOC3COC3)C[C@@H]3CC[C@@H](C)[C@@](O)(O3)C(=O)C(=O)N3CCCC[C@H]3C(=O)O2)C[C@H]1OC] |
| I-70 | 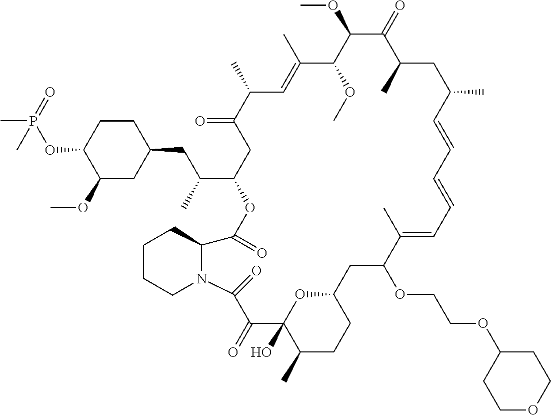 [SMILES: CO[C@@H]1/C(C)=C/[C@@H](C)C(=O)C[C@@H]([C@H](C)C[C@@H]2CC[C@@H](OP(C)(C)=O)[C@H](OC)C2)OC(=O)[C@@H]2CCCCN2C(=O)C(=O)[C@]2(O)O[C@@H](CC[C@H]2C)CC(OCCOC2CCOCC2)/C(C)=C/C=C/C=C/[C@@H](C)C[C@@H](C)C(=O)[C@@H]1OC] |
| I-71 | 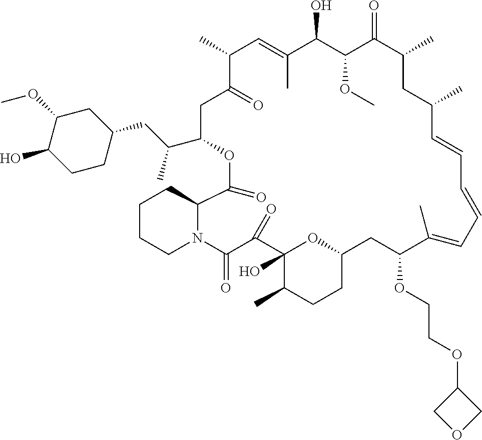 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OCCOC3COC3)C[C@@H]3CC[C@@H](C)[C@@](O)(O3)C(=O)C(=O)N3CCCC[C@H]3C(=O)O2)CC[C@H]1O] |
| I-72 | 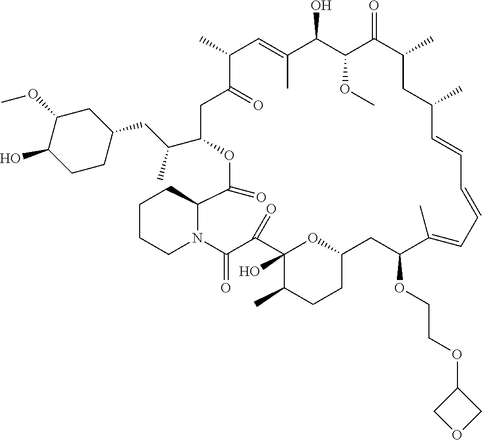 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OCCOC3COC3)C[C@@H]3CC[C@@H](C)[C@@](O)(O3)C(=O)C(=O)N3CCCC[C@H]3C(=O)O2)CC[C@H]1O] |
| I-73 | 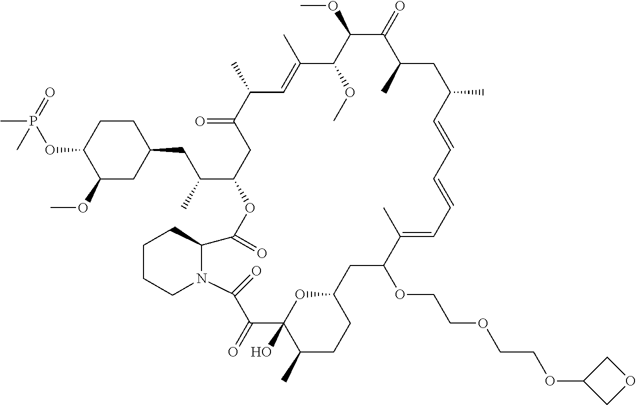 [SMILES: CO[C@@H]1/C(C)=C/[C@@H](C)C(=O)C[C@@H]([C@H](C)C[C@@H]2CC[C@@H](OP(C)(C)=O)[C@H](OC)C2)OC(=O)[C@@H]2CCCCN2C(=O)C(=O)[C@]2(O)O[C@@H](CC[C@H]2C)CC(OCCOCCOC2COC2)/C(C)=C/C=C/C=C/[C@@H](C)C[C@@H](C)C(=O)[C@@H]1OC] |
| I-74 | 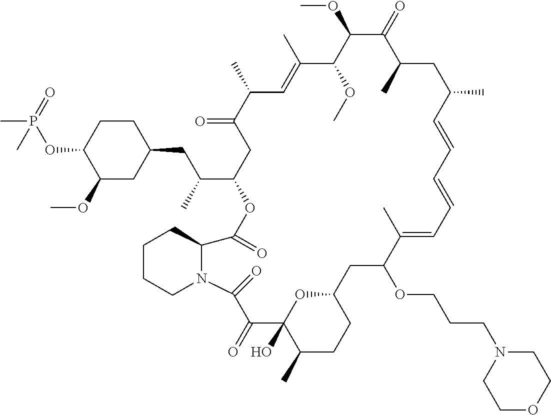 [SMILES: CO[C@@H]1/C(C)=C/[C@@H](C)C(=O)C[C@@H]([C@H](C)C[C@@H]2CC[C@@H](OP(C)(C)=O)[C@H](OC)C2)OC(=O)[C@@H]2CCCCN2C(=O)C(=O)[C@]2(O)O[C@@H](CC[C@H]2C)CC(OCCCN2CCOCC2)/C(C)=C/C=C/C=C/[C@@H](C)C[C@@H](C)C(=O)[C@@H]1OC] |
| I-75 | 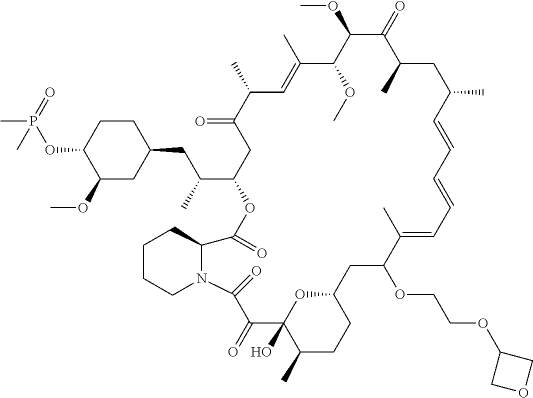 [SMILES: CO[C@@H]1/C(C)=C/[C@@H](C)C(=O)C[C@@H]([C@H](C)C[C@@H]2CC[C@@H](OP(C)(C)=O)[C@H](OC)C2)OC(=O)[C@@H]2CCCCN2C(=O)C(=O)[C@]2(O)O[C@@H](CC[C@H]2C)CC(OCCOC2COC2)/C(C)=C/C=C/C=C/[C@@H](C)C[C@@H](C)C(=O)[C@@H]1OC] |
| I-76 | 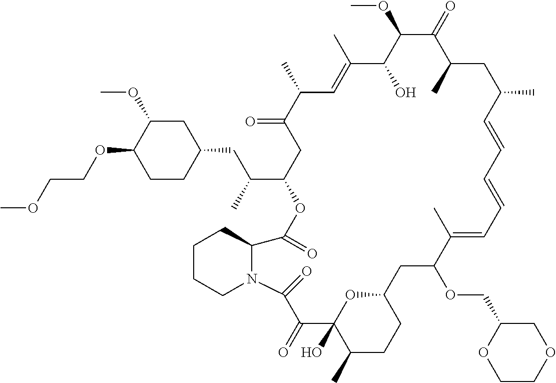 [SMILES: COCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OC[C@@H]3COCCO3)C[C@@H]3CC[C@@H](C)[C@@](O)(O3)C(=O)C(=O)N3CCCC[C@H]3C(=O)O2)C[C@H]1OC] |
| I-77 | 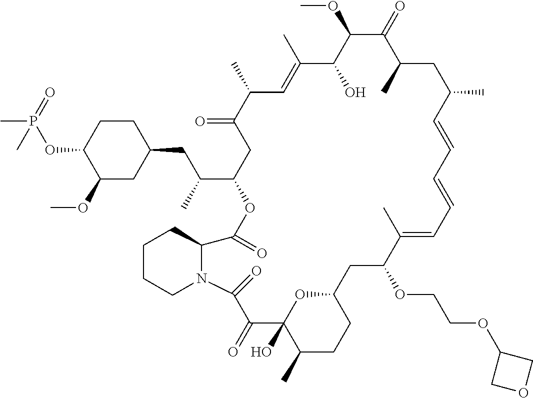 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OCCOC3COC3)C[C@@H]3CC[C@@H](C)[C@@](O)(O3)C(=O)C(=O)N3CCCC[C@H]3C(=O)O2)CC[C@H]1OP(C)(C)=O] |
| I-78 | 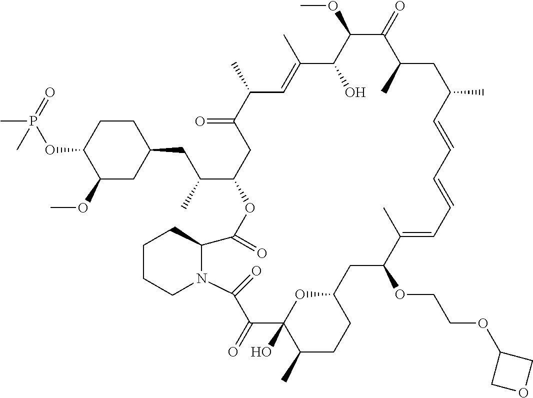 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OCCOC3COC3)C[C@@H]3CC[C@@H](C)[C@@](O)(O3)C(=O)C(=O)N3CCCC[C@H]3C(=O)O2)CC[C@H]1OP(C)(C)=O] |
| I-79 | 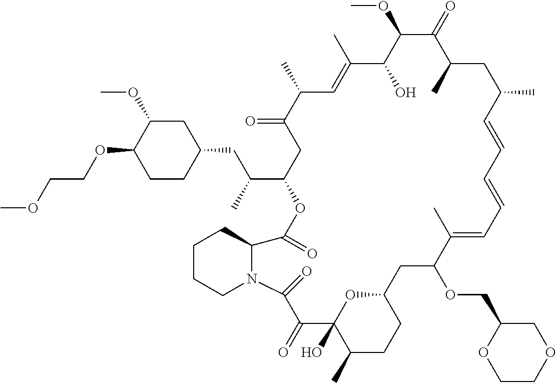 [SMILES: COCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OC[C@H]3COCCO3)C[C@@H]3CC[C@@H](C)[C@@](O)(O3)C(=O)C(=O)N3CCCC[C@H]3C(=O)O2)C[C@H]1OC] |
| I-80 | 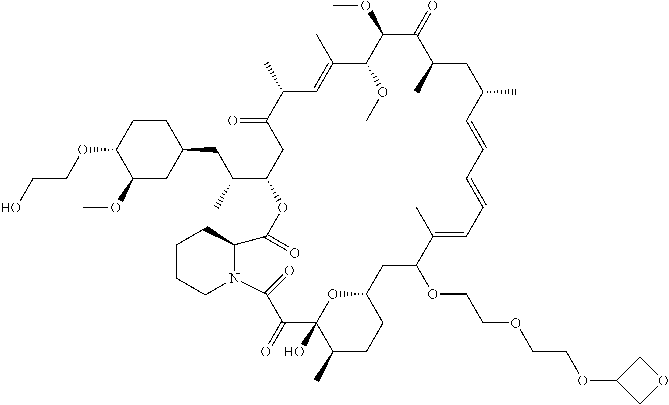 [SMILES: CO[C@@H]1/C(C)=C/[C@@H](C)C(=O)C[C@@H]([C@H](C)C[C@@H]2CC[C@@H](OCCO)[C@H](OC)C2)OC(=O)[C@@H]2CCCCN2C(=O)C(=O)[C@]2(O)O[C@@H](CC[C@H]2C)CC(OCCOCCOC2COC2)/C(C)=C/C=C/C=C/[C@@H](C)C[C@@H](C)C(=O)[C@@H]1OC] |
| I-81 | 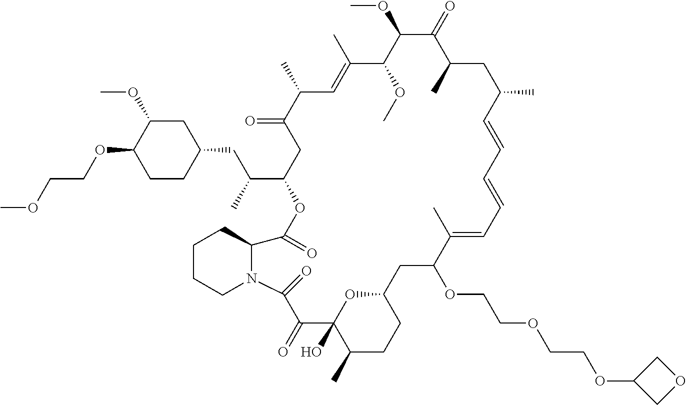 [SMILES: COCCO[C@@H]1CC[C@@H](C[C@@H](C)[C@@H]2CC(=O)[C@H](C)/C=C(\C)[C@@H](OC)[C@@H](OC)C(=O)[C@H](C)C[C@H](C)/C=C/C=C/C=C(\C)C(OCCOCCOC3COC3)C[C@@H]3CC[C@@H](C)[C@@](O)(O3)C(=O)C(=O)N3CCCC[C@H]3C(=O)O2)C[C@H]1OC] |
| I-82 | 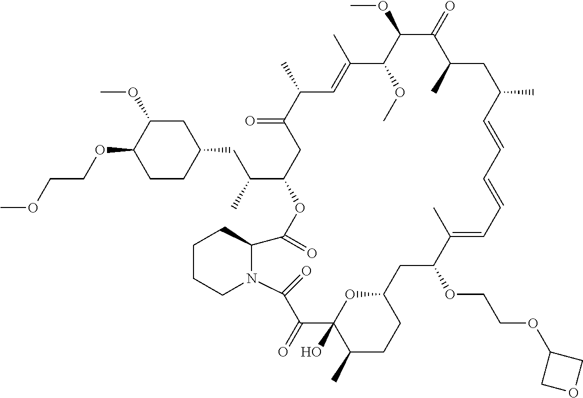 [SMILES: COCCO[C@@H]1CC[C@@H](C[C@@H](C)[C@@H]2CC(=O)[C@H](C)/C=C(\C)[C@@H](OC)[C@@H](OC)C(=O)[C@H](C)C[C@H](C)/C=C/C=C/C=C(\C)[C@H](OCCOC3COC3)C[C@@H]3CC[C@@H](C)[C@@](O)(O3)C(=O)C(=O)N3CCCC[C@H]3C(=O)O2)C[C@H]1OC] |
| I-83 | 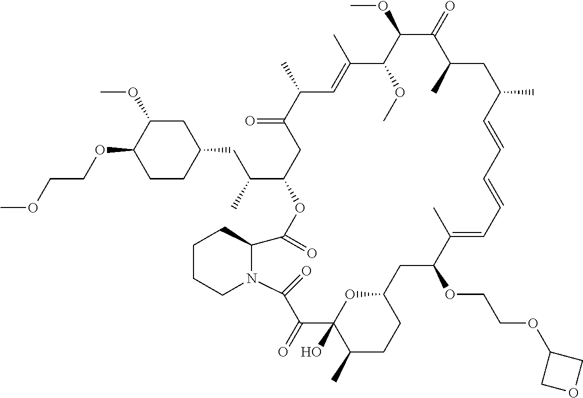 [SMILES: COCCO[C@@H]1CC[C@@H](C[C@@H](C)[C@@H]2CC(=O)[C@H](C)/C=C(\C)[C@@H](OC)[C@@H](OC)C(=O)[C@H](C)C[C@H](C)/C=C/C=C/C=C(\C)[C@@H](OCCOC3COC3)C[C@@H]3CC[C@@H](C)[C@@](O)(O3)C(=O)C(=O)N3CCCC[C@H]3C(=O)O2)C[C@H]1OC] |
| I-84 | 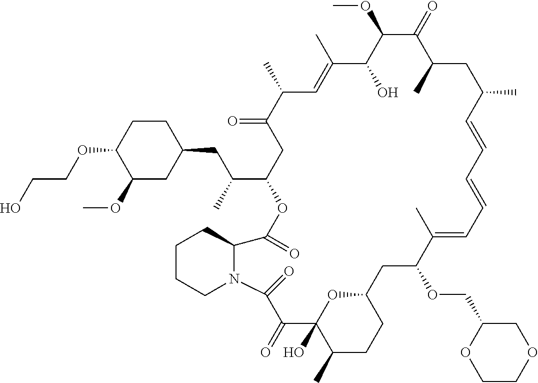 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OC[C@@H]3COCCO3)C[C@@H]3CC[C@@H](C)[C@@](O)(O3)C(=O)C(=O)N3CCCC[C@H]3C(=O)O2)CC[C@H]1OCCO] |
| I-85 | 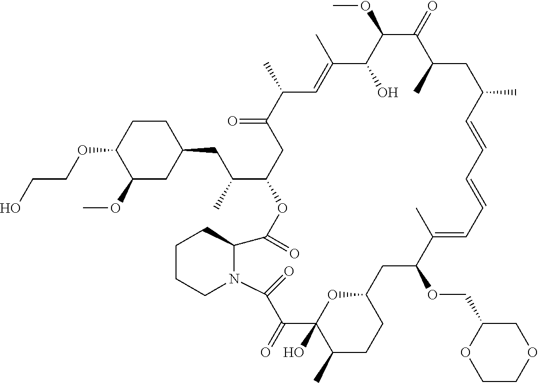 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC[C@@H]3COCCO3)C[C@@H]3CC[C@@H](C)[C@@](O)(O3)C(=O)C(=O)N3CCCC[C@H]3C(=O)O2)CC[C@H]1OCCO] |
| I-86 | 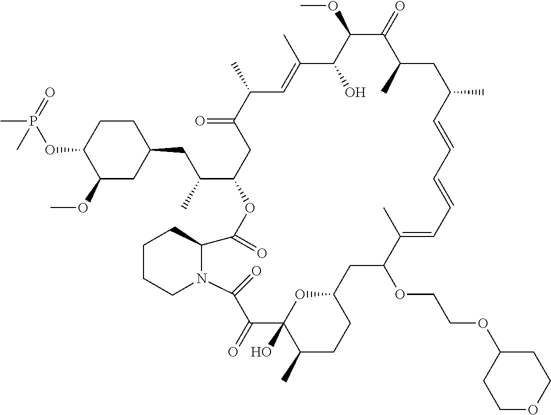 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCCOC3CCOCC3)C[C@@H]3CC[C@@H](C)[C@@](O)(O3)C(=O)C(=O)N3CCCC[C@H]3C(=O)O2)CC[C@H]1OP(C)(C)=O] |
| I-87 | 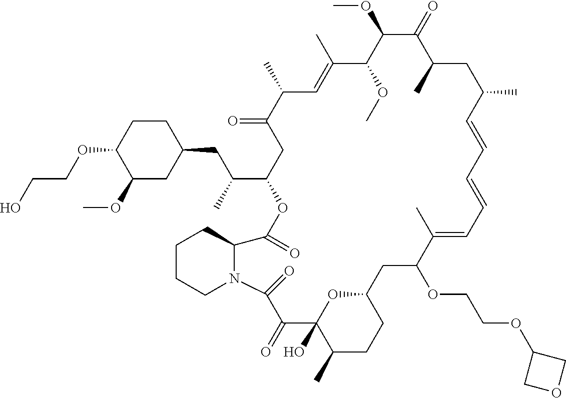 [SMILES: CO[C@@H]1/C(C)=C/[C@@H](C)C(=O)C[C@@H]([C@H](C)C[C@@H]2CC[C@@H](OCCO)[C@H](OC)C2)OC(=O)[C@@H]2CCCCN2C(=O)C(=O)[C@]2(O)O[C@@H](CC[C@H]2C)CC(OCCOC2COC2)/C(C)=C/C=C/C=C/[C@@H](C)C[C@@H](C)C(=O)[C@@H]1OC] |
| I-88 | 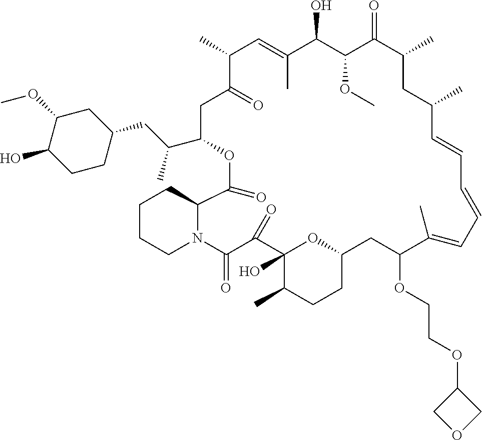 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCCOC3COC3)C[C@@H]3CC[C@@H](C)[C@@](O)(O3)C(=O)C(=O)N3CCCC[C@H]3C(=O)O2)CC[C@H]1O] |
| I-89 | 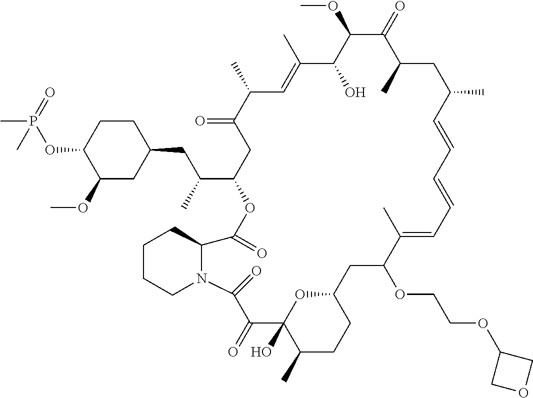 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCCOC3COC3)C[C@@H]3CC[C@@H](C)[C@@](O)(O3)C(=O)C(=O)N3CCCC[C@H]3C(=O)O2)CC[C@H]1OP(C)(C)=O] |
| I-90 | 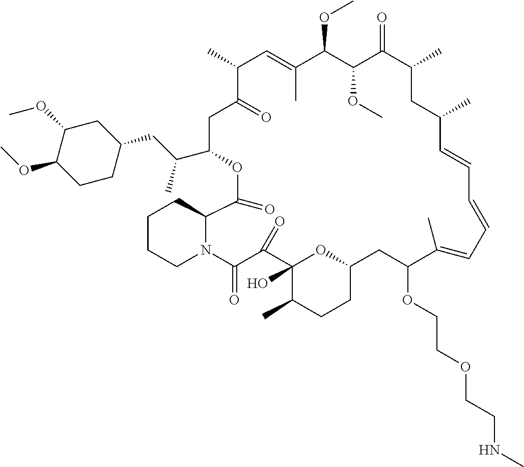 |
| I-91 | 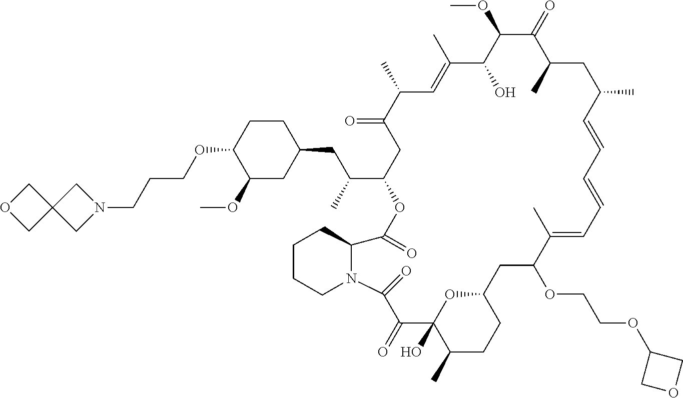 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCCOC3COC3)C[C@@H]3CC[C@@H](C)[C@@](O)(O3)C(=O)C(=O)N3CCCC[C@H]3C(=O)O2)CC[C@H]1OCCCN1CC2(COC2)C1] |
| I-92 | 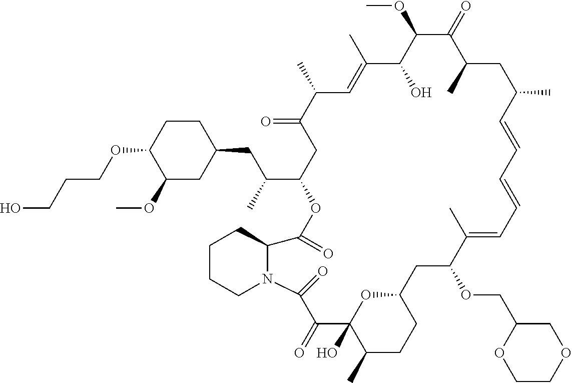 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OCC3COCCO3)C[C@@H]3CC[C@@H](C)[C@@](O)(O3)C(=O)C(=O)N3CCCC[C@H]3C(=O)O2)CC[C@H]1OCCCO] |
| I-93 | 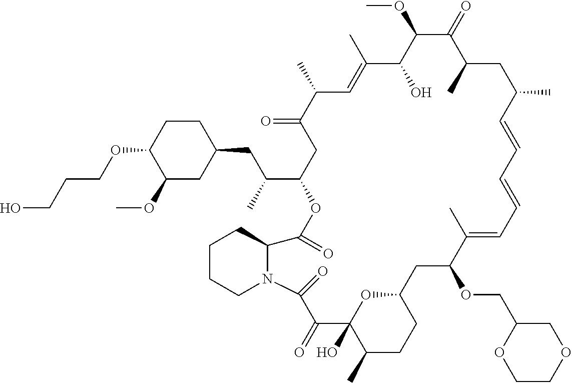 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OCC3COCCO3)C[C@@H]3CC[C@@H](C)[C@@](O)(O3)C(=O)C(=O)N3CCCC[C@H]3C(=O)O2)CC[C@H]1OCCCO] |
| I-94 | 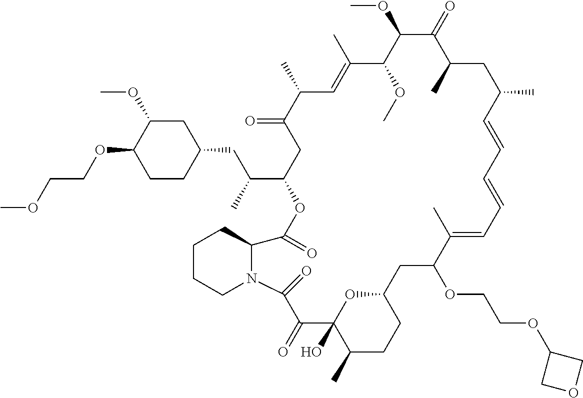 [SMILES: COCCO[C@@H]1CC[C@@H](C[C@@H](C)[C@@H]2CC(=O)[C@H](C)/C=C(\C)[C@@H](OC)[C@@H](OC)C(=O)[C@H](C)C[C@H](C)/C=C/C=C/C=C(\C)C(OCCOC3COC3)C[C@@H]3CC[C@@H](C)[C@@](O)(O3)C(=O)C(=O)N3CCCC[C@H]3C(=O)O2)C[C@H]1OC] |
| I-95 | 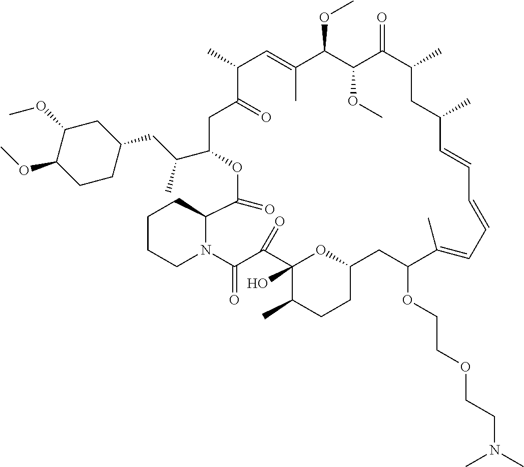 |
| I-96 | 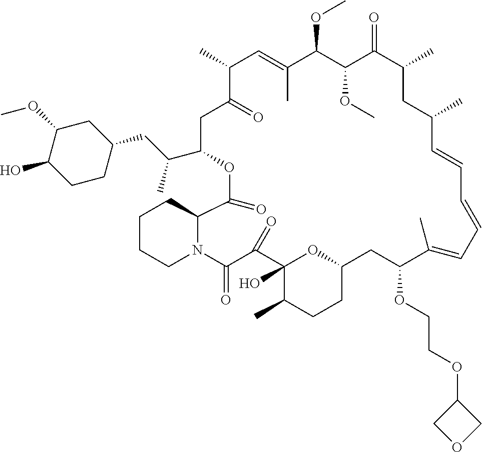 [SMILES: CO[C@@H]1/C(C)=C/[C@@H](C)C(=O)C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)C[C@@H](OCCOC2COC2)/C(C)=C/C=C/C=C/[C@@H](C)C[C@@H](C)C(=O)[C@@H]1OC] |
| I-97 | 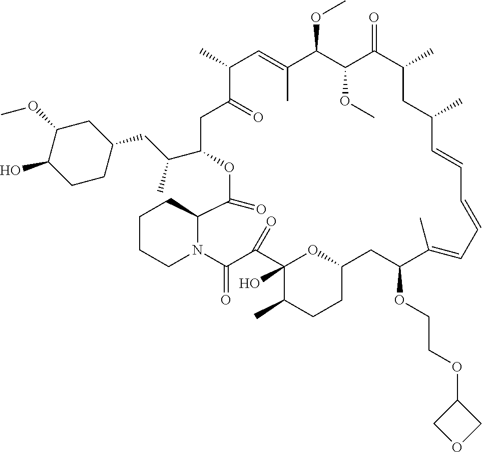 [SMILES: CO[C@@H]1/C(C)=C/[C@@H](C)C(=O)C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)C[C@H](OCCOC2COC2)/C(C)=C/C=C/C=C/[C@@H](C)C[C@@H](C)C(=O)[C@@H]1OC] |
| I-99 | 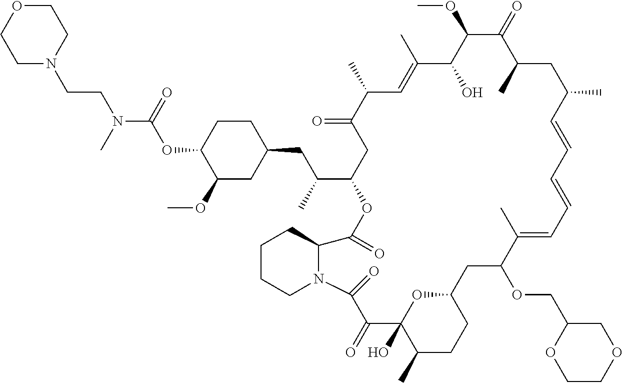 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCC3COCCO3)C[C@@H]3CC[C@@H](C)[C@@](O)(O3)C(=O)C(=O)N3CCCC[C@H]3C(=O)O2)CC[C@H]1OC(=O)N(C)CCN1CCOCC1] |
| I-100 | 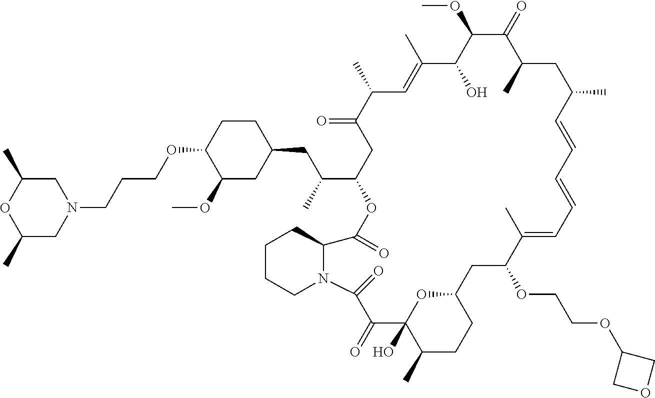 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OCCOC3COC3)C[C@@H]3CC[C@@H](C)[C@@](O)(O3)C(=O)C(=O)N3CCCC[C@H]3C(=O)O2)CC[C@H]1OCCCN1C[C@@H](C)O[C@@H](C)C1] |
| I-101 | 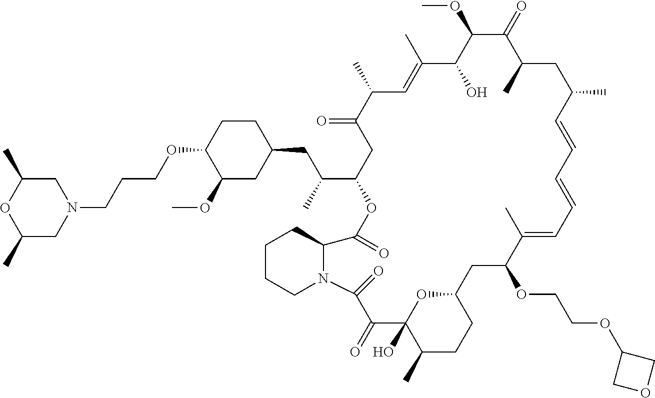 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OCCOC3COC3)C[C@@H]3CC[C@@H](C)[C@@](O)(O3)C(=O)C(=O)N3CCCC[C@H]3C(=O)O2)CC[C@H]1OCCCN1C[C@@H](C)O[C@@H](C)C1] |
| I-102 | 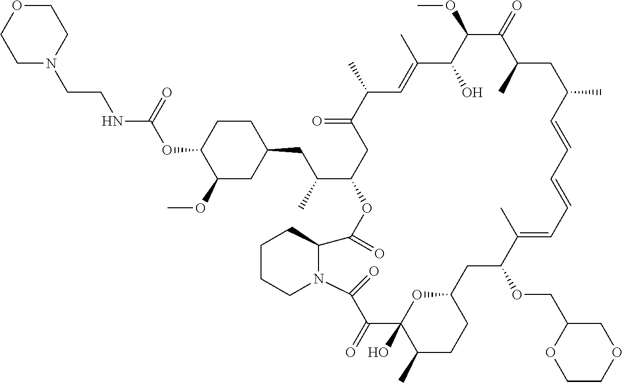 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OCC3COCCO3)C[C@@H]3CC[C@@H](C)[C@@](O)(O3)C(=O)C(=O)N3CCCC[C@H]3C(=O)O2)CC[C@H]1OC(=O)NCCN1CCOCC1] |
| I-103 | 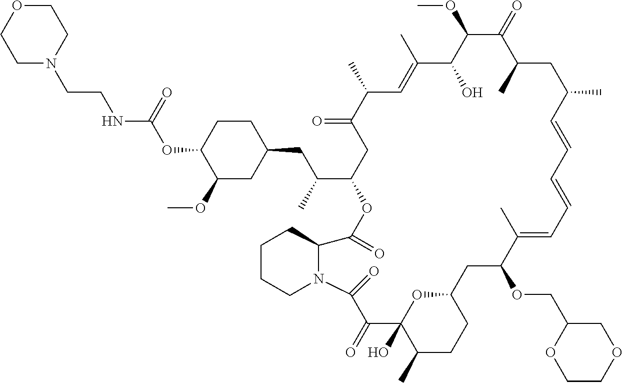 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OCC3COCCO3)C[C@@H]3CC[C@@H](C)[C@@](O)(O3)C(=O)C(=O)N3CCCC[C@H]3C(=O)O2)CC[C@H]1OC(=O)NCCN1CCOCC1] |
| I-104 | 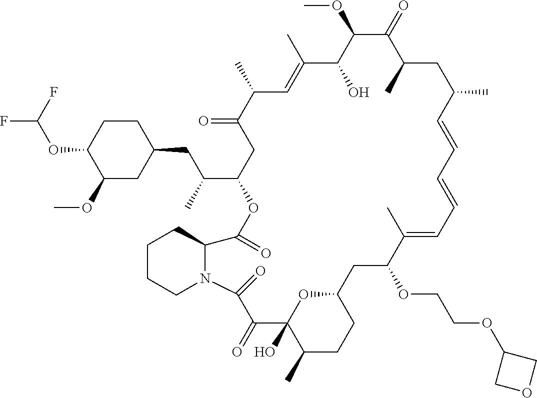 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OCCOC3COC3)C[C@@H]3CC[C@@H](C)[C@@](O)(O3)C(=O)C(=O)N3CCCC[C@H]3C(=O)O2)CC[C@H]1OC(F)F] |
| I-105 | 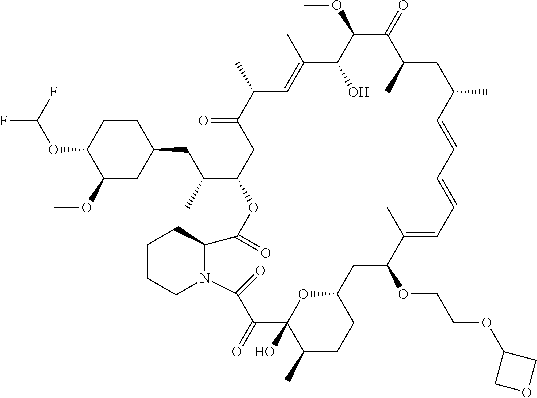 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OCCOC3COC3)C[C@@H]3CC[C@@H](C)[C@@](O)(O3)C(=O)C(=O)N3CCCC[C@H]3C(=O)O2)CC[C@H]1OC(F)F] |
| I-106 | 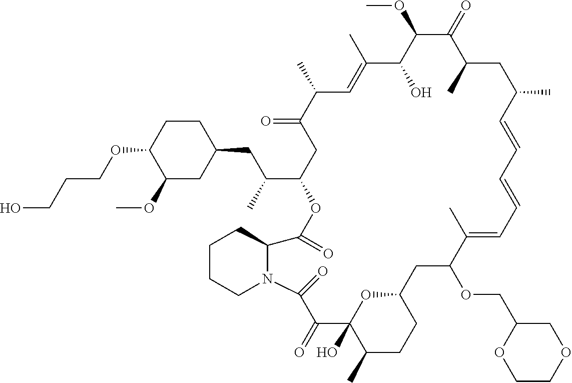 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCC3COCCO3)C[C@@H]3CC[C@@H](C)[C@@](O)(O3)C(=O)C(=O)N3CCCC[C@H]3C(=O)O2)CC[C@H]1OCCCO] |
| I-107 | 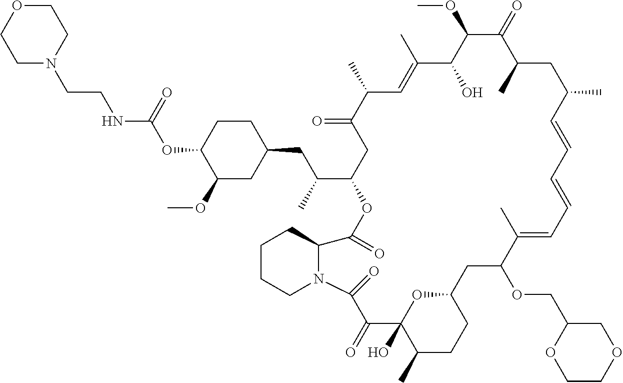 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCC3COCCO3)C[C@@H]3CC[C@@H](C)[C@@](O)(O3)C(=O)C(=O)N3CCCC[C@H]3C(=O)O2)CC[C@H]1OC(=O)NCCN1CCOCC1] |
| I-108 | 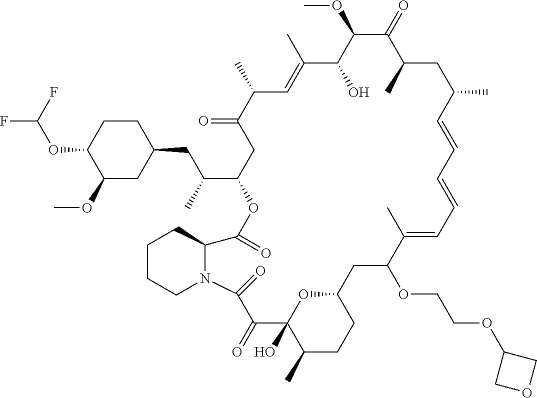 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCCOC3COC3)C[C@@H]3CC[C@@H](C)[C@@](O)(O3)C(=O)C(=O)N3CCCC[C@H]3C(=O)O2)CC[C@H]1OC(F)F] |
| I-109 | 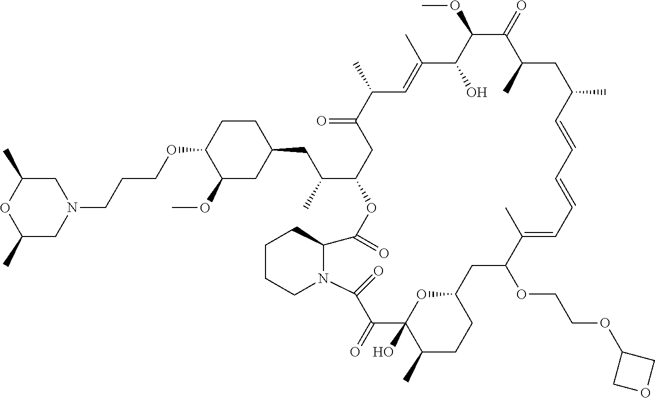 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(OCCOC3COC3)C[C@@H]3CC[C@@H](C)[C@@](O)(O3)C(=O)C(=O)N3CCCC[C@H]3C(=O)O2)CC[C@H]1OCCCN1C[C@@H](C)O[C@@H](C)C1] |
| I-110 | 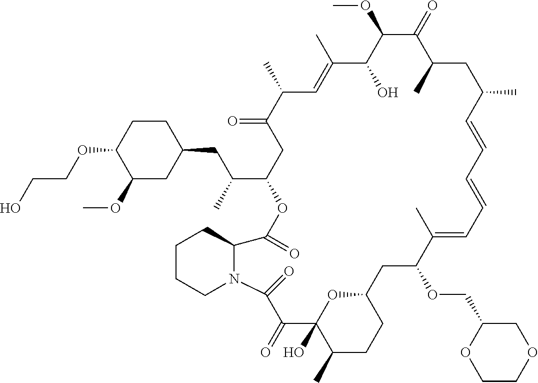 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@H](OC[C@@H]3COCCO3)C[C@@H]3CC[C@@H](C)[C@@](O)(O3)C(=O)C(=O)N3CCCC[C@H]3C(=O)O2)CC[C@H]1OCCO] |

In some embodiments, the present invention provides a compound set forth in Table 1, above, or a pharmaceutically acceptable salt thereof. In some embodiments, the present invention provides a compound set forth in Table 1, above, or a pharmaceutically acceptable salt, stereoisomers, tautomers, and polymorphs thereof. In some embodiments, the present invention provides a compound set forth in Table 1, above, include the replacement of one or more hydrogens by deuterium. It will be appreciated that the present invention also provides a compound set forth in Table 1, above, as a racemic mixture at the C7 position, or a pharmaceutically acceptable salt thereof. Further, it will be appreciated that compounds set forth in Table 1, above, as racemic mixtures at the C7 hydroxyl position may be separated into diastereomers by various methods, e.g., chiral chromatography.

In some embodiments, the present invention provides a compound of Formula I or I′, or pharmaceutically acceptable salt thereof, wherein when:

### L² is

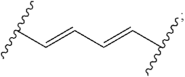 [SMILES: */C=C/C=C/*]

R³ and R^{3′} are hydrogen;
R⁵ and R^{5′} are taken together to form ═O;
and X¹ and X² are both —CH₂—; then:
—X-L¹-R¹, —X³—R², R⁴, and R⁶ are present in a combination other than those combinations presented in below in each row of Table 1A.

TABLE 1A

| No. | | | | |
|---|---|---|---|---|
| # | -X³-R² | R⁶ | R⁴ | -X-L¹-R¹ |
| 1 | 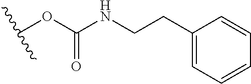 [SMILES: *OC(=O)NCCc1ccccc1] | 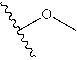 [SMILES: *OC] | 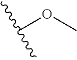 [SMILES: *OC] | 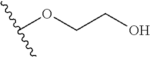 [SMILES: *OCCO] |
| 2 | 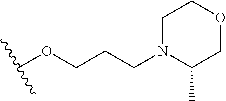 [SMILES: *OCCCN1CCOC[C@@H]1C] | 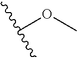 [SMILES: *OC] | 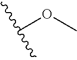 [SMILES: *OC] | 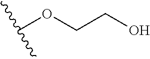 [SMILES: *OCCO] |
| 3 | 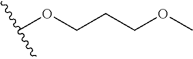 [SMILES: *OCCCOC] | 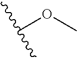 [SMILES: *OC] | 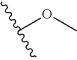 [SMILES: *OC] | 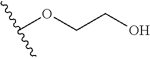 [SMILES: *OCCO] |
| 4 | 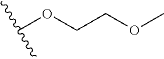 [SMILES: *OCCOC] | 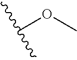 [SMILES: *OC] | 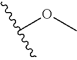 [SMILES: *OC] | 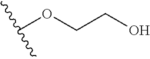 [SMILES: *OCCO] |
| 5 | 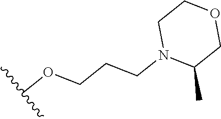 [SMILES: *OCCCN1CCOC[C@H]1C] | 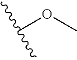 [SMILES: *OC] | 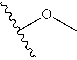 [SMILES: *OC] | 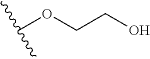 [SMILES: *OCCO] |
| 6 | 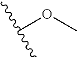 [SMILES: *OC] | 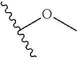 [SMILES: *OC] | 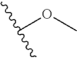 [SMILES: *OC] | 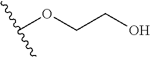 [SMILES: *OCCO] |
| 7 | 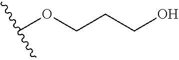 [SMILES: *OCCCO] | 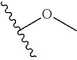 [SMILES: *OC] | 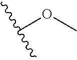 [SMILES: *OC] | 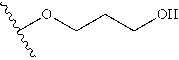 [SMILES: *OCCCO] |
| 8 | 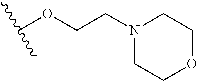 [SMILES: *OCCN1CCOCC1] | 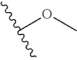 [SMILES: *OC] | 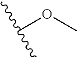 [SMILES: *OC] | 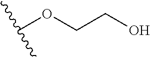 [SMILES: *OCCO] |
| 9 | 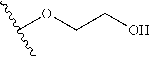 [SMILES: *OCCO] | 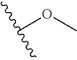 [SMILES: *OC] | 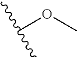 [SMILES: *OC] | 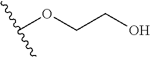 [SMILES: *OCCO] |
| 10 | 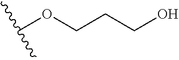 [SMILES: *OCCCO] | 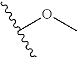 [SMILES: *OC] | 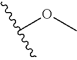 [SMILES: *OC] | 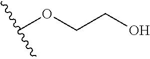 [SMILES: *OCCO] |
| 11 | 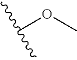 [SMILES: *OC] | 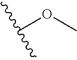 [SMILES: *OC] | 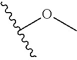 [SMILES: *OC] | 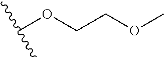 [SMILES: *OCCOC] |
| 12 | 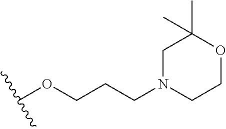 [SMILES: *OCCCN1CCOC(C)(C)C1] | 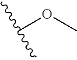 [SMILES: *OC] | 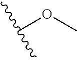 [SMILES: *OC] | 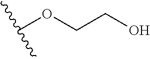 [SMILES: *OCCO] |
| 13 | 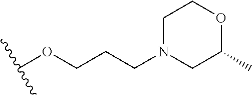 [SMILES: *OCCCN1CCO[C@H](C)C1] | 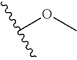 [SMILES: *OC] | 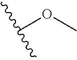 [SMILES: *OC] | 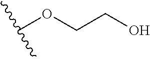 [SMILES: *OCCO] |
| 14 | 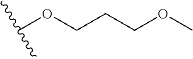 [SMILES: *OCCCOC] | 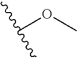 [SMILES: *OC] | 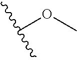 [SMILES: *OC] | 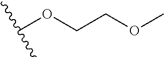 [SMILES: *OCCOC] |
| 15 | 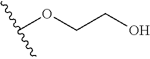 [SMILES: *OCCO] | 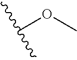 [SMILES: *OC] | 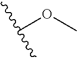 [SMILES: *OC] | 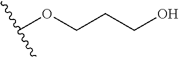 [SMILES: *OCCCO] |
| 16 | 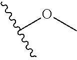 [SMILES: *OC] | 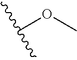 [SMILES: *OC] | 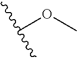 [SMILES: *OC] | 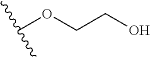 [SMILES: *OCCO] |
| 17 | 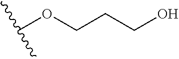 [SMILES: *OCCCO] | 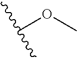 [SMILES: *OC] | 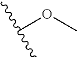 [SMILES: *OC] | 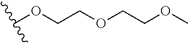 [SMILES: *OCCOCCOC] |
| 18 | 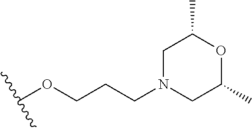 [SMILES: *OCCCN1C[C@@H](C)O[C@@H](C)C1] | 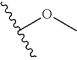 [SMILES: *OC] | 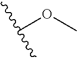 [SMILES: *OC] | 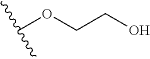 [SMILES: *OCCO] |
| 19 | 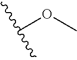 [SMILES: *OC] | 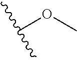 [SMILES: *OC] | 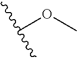 [SMILES: *OC] | 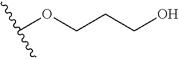 [SMILES: *OCCCO] |
| 20 | 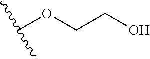 [SMILES: *OCCO] | 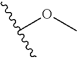 [SMILES: *OC] | 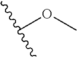 [SMILES: *OC] | 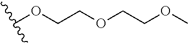 [SMILES: *OCCOCCOC] |
| 21 | 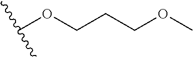 [SMILES: *OCCCOC] | 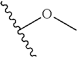 [SMILES: *OC] | 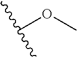 [SMILES: *OC] | 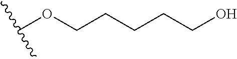 [SMILES: *OCCCCCO] |
| 22 | 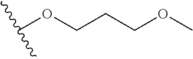 [SMILES: *OCCCOC] | 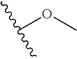 [SMILES: *OC] | 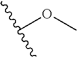 [SMILES: *OC] | 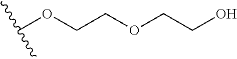 [SMILES: *OCCOCCO] |
| 24 | 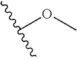 [SMILES: *OC] | 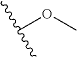 [SMILES: *OC] | 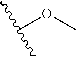 [SMILES: *OC] | 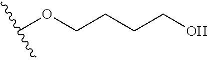 [SMILES: *OCCCCO] |
| 25 | 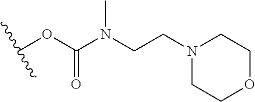 [SMILES: *OC(=O)N(C)CCN1CCOCC1] | 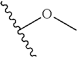 [SMILES: *OC] | 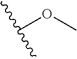 [SMILES: *OC] | 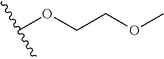 [SMILES: *OCCOC] |
| 26 | 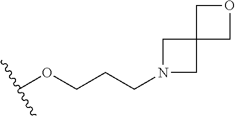 [SMILES: *OCCCN1CC2(COC2)C1] | 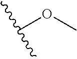 [SMILES: *OC] | 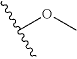 [SMILES: *OC] | 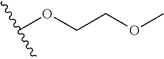 [SMILES: *OCCOC] |
| 27 | 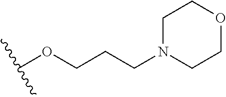 [SMILES: *OCCCN1CCOCC1] | 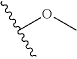 [SMILES: *OC] | 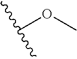 [SMILES: *OC] | 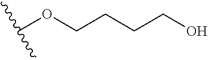 [SMILES: *OCCCCO] |
| 28 | 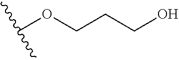 [SMILES: *OCCCO] | 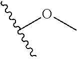 [SMILES: *OC] | 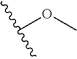 [SMILES: *OC] | 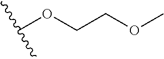 [SMILES: *OCCOC] |
| 29 | 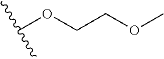 [SMILES: *OCCOC] | 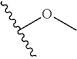 [SMILES: *OC] | 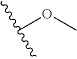 [SMILES: *OC] | 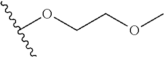 [SMILES: *OCCOC] |
| 30 | 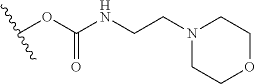 [SMILES: *OC(=O)NCCN1CCOCC1] | 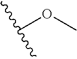 [SMILES: *OC] | 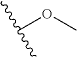 [SMILES: *OC] | 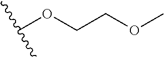 [SMILES: *OCCOC] |
| 31 | 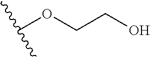 [SMILES: *OCCO] | 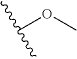 [SMILES: *OC] | 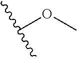 [SMILES: *OC] | 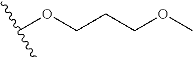 [SMILES: *OCCCOC] |
| 32 | 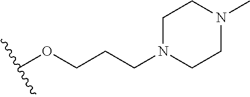 [SMILES: *OCCCN1CCN(C)CC1] | 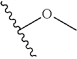 [SMILES: *OC] | 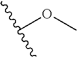 [SMILES: *OC] | 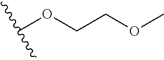 [SMILES: *OCCOC] |
| 33 | 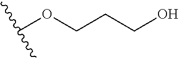 [SMILES: *OCCCO] | 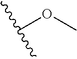 [SMILES: *OC] | 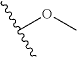 [SMILES: *OC] | 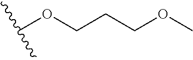 [SMILES: *OCCCOC] |
| 34 | 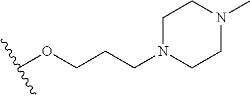 [SMILES: *OCCCN1CCN(C)CC1] | 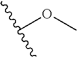 [SMILES: *OC] | 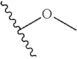 [SMILES: *OC] | 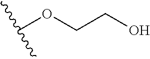 [SMILES: *OCCO] |
| 35 | 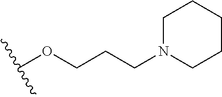 [SMILES: *OCCCN1CCCCC1] | 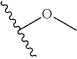 [SMILES: *OC] | 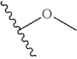 [SMILES: *OC] | 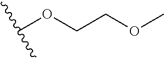 [SMILES: *OCCOC] |
| 36 | 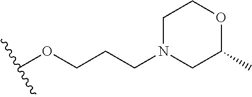 [SMILES: *OCCCN1CCO[C@H](C)C1] | 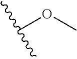 [SMILES: *OC] | 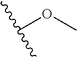 [SMILES: *OC] | 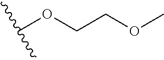 [SMILES: *OCCOC] |
| 37 | 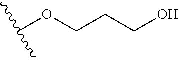 [SMILES: *OCCCO] | 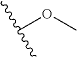 [SMILES: *OC] | 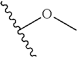 [SMILES: *OC] | 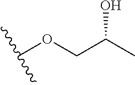 [SMILES: *OC[C@@H](C)O] |
| 24 | 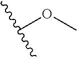 [SMILES: *OC] | 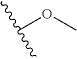 [SMILES: *OC] | 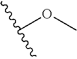 [SMILES: *OC] | 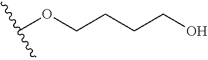 [SMILES: *OCCCCO] |
| 25 | 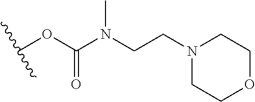 [SMILES: *OC(=O)N(C)CCN1CCOCC1] | 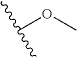 [SMILES: *OC] | 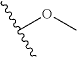 [SMILES: *OC] | 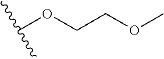 [SMILES: *OCCOC] |
| 26 | 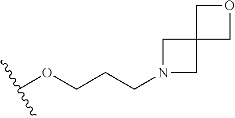 [SMILES: *OCCCN1CC2(COC2)C1] | 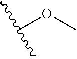 [SMILES: *OC] | 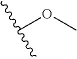 [SMILES: *OC] | 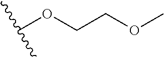 [SMILES: *OCCOC] |
| 27 | 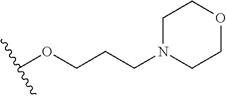 [SMILES: *OCCCN1CCOCC1] | 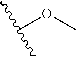 [SMILES: *OC] | 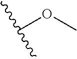 [SMILES: *OC] | 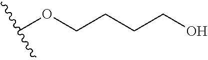 [SMILES: *OCCCCO] |
| 28 | 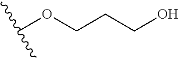 [SMILES: *OCCCO] | 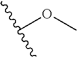 [SMILES: *OC] | 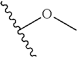 [SMILES: *OC] | 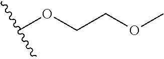 [SMILES: *OCCOC] |
| 29 | 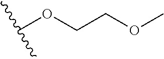 [SMILES: *OCCOC] | 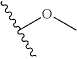 [SMILES: *OC] | 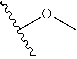 [SMILES: *OC] | 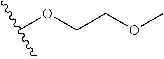 [SMILES: *OCCOC] |
| 30 | 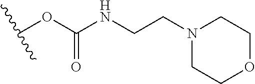 [SMILES: *OC(=O)NCCN1CCOCC1] | 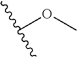 [SMILES: *OC] | 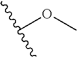 [SMILES: *OC] | 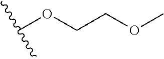 [SMILES: *OCCOC] |
| 31 | 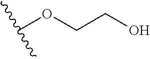 [SMILES: *OCCO] | 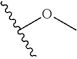 [SMILES: *OC] | 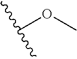 [SMILES: *OC] | 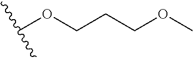 [SMILES: *OCCCOC] |
| 32 | 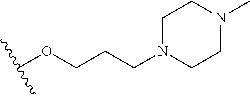 [SMILES: *OCCCN1CCN(C)CC1] | 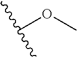 [SMILES: *OC] | 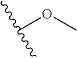 [SMILES: *OC] | 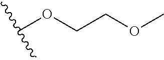 [SMILES: *OCCOC] |
| 33 | 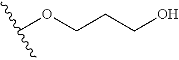 [SMILES: *OCCCO] | 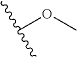 [SMILES: *OC] | 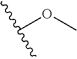 [SMILES: *OC] | 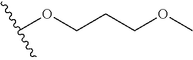 [SMILES: *OCCCOC] |
| 34 | 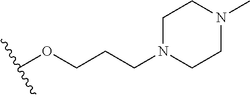 [SMILES: *OCCCN1CCN(C)CC1] | 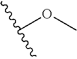 [SMILES: *OC] | 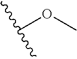 [SMILES: *OC] | 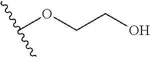 [SMILES: *OCCO] |
| 35 | 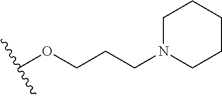 [SMILES: *OCCCN1CCCCC1] | 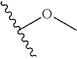 [SMILES: *OC] | 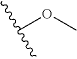 [SMILES: *OC] | 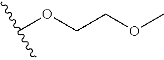 [SMILES: *OCCOC] |
| 36 | 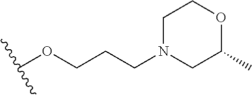 [SMILES: *OCCCN1CCO[C@H](C)C1] | 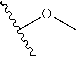 [SMILES: *OC] | 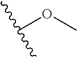 [SMILES: *OC] | 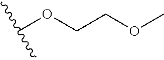 [SMILES: *OCCOC] |
| 37 | 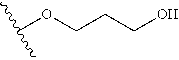 [SMILES: *OCCCO] | 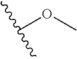 [SMILES: *OC] | 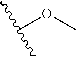 [SMILES: *OC] | 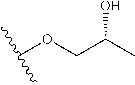 [SMILES: *OC[C@@H](C)O] |
| 38 | 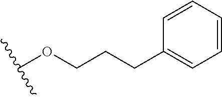 [SMILES: *OCCCc1ccccc1] | 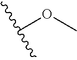 [SMILES: *OC] | 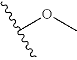 [SMILES: *OC] | 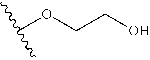 [SMILES: *OCCO] |
| 39 | 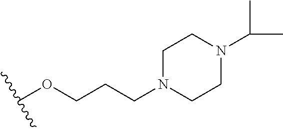 [SMILES: *OCCCN1CCN(C(C)C)CC1] | 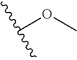 [SMILES: *OC] | 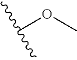 [SMILES: *OC] | 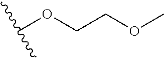 [SMILES: *OCCOC] |
| 40 | 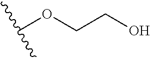 [SMILES: *OCCO] | 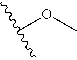 [SMILES: *OC] | 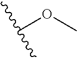 [SMILES: *OC] | 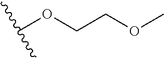 [SMILES: *OCCOC] |
| 41 | 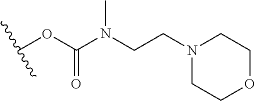 [SMILES: *OC(=O)N(C)CCN1CCOCC1] | 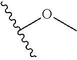 [SMILES: *OC] | 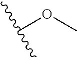 [SMILES: *OC] | 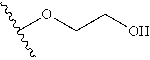 [SMILES: *OCCO] |
| 42 | 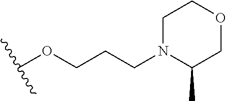 [SMILES: *OCCCN1CCOC[C@H]1C] | 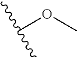 [SMILES: *OC] | 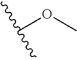 [SMILES: *OC] | 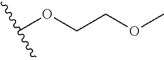 [SMILES: *OCCOC] |
| 43 | 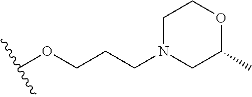 [SMILES: *OCCCN1CCO[C@H](C)C1] | 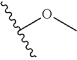 [SMILES: *OC] | 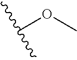 [SMILES: *OC] | 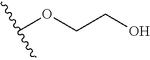 [SMILES: *OCCO] |
| 44 | 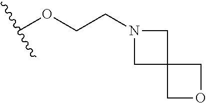 [SMILES: *OCCN1CC2(COC2)C1] | 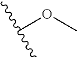 [SMILES: *OC] | 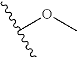 [SMILES: *OC] | 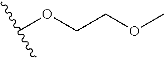 [SMILES: *OCCOC] |
| 45 | 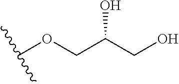 [SMILES: *OC[C@H](O)CO] | 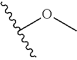 [SMILES: *OC] | 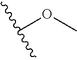 [SMILES: *OC] | 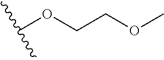 [SMILES: *OCCOC] |
| 46 | 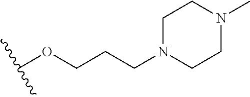 [SMILES: *OCCCN1CCN(C)CC1] | 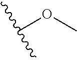 [SMILES: *OC] | 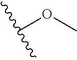 [SMILES: *OC] | 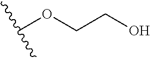 [SMILES: *OCCO] |
| 47 | 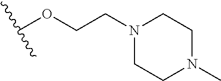 [SMILES: *OCCN1CCN(C)CC1] | 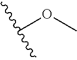 [SMILES: *OC] | 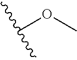 [SMILES: *OC] | 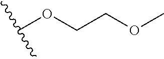 [SMILES: *OCCOC] |
| 48 | 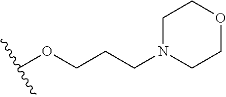 [SMILES: *OCCCN1CCOCC1] | 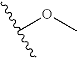 [SMILES: *OC] | 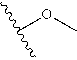 [SMILES: *OC] | 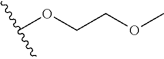 [SMILES: *OCCOC] |
| 49 | 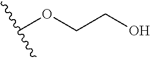 [SMILES: *OCCO] | 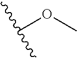 [SMILES: *OC] | 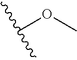 [SMILES: *OC] | 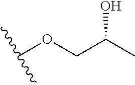 [SMILES: *OC[C@@H](C)O] |
| 50 | 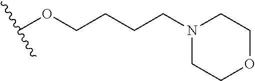 [SMILES: *OCCCCN1CCOCC1] | 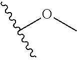 [SMILES: *OC] | 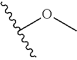 [SMILES: *OC] | 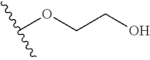 [SMILES: *OCCO] |
| 51 | 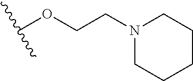 [SMILES: *OCCN1CCCCC1] | 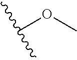 [SMILES: *OC] | 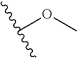 [SMILES: *OC] | 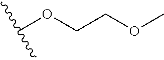 [SMILES: *OCCOC] |
| 52 | 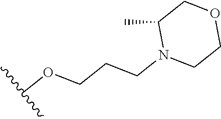 [SMILES: *OCCCN1CCOC[C@H]1C] | 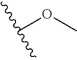 [SMILES: *OC] | 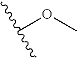 [SMILES: *OC] | 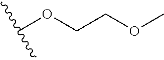 [SMILES: *OCCOC] |
| 53 | 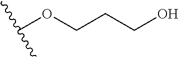 [SMILES: *OCCCO] | 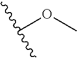 [SMILES: *OC] | 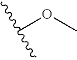 [SMILES: *OC] | 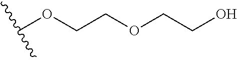 [SMILES: *OCCOCCO] |
| 54 | 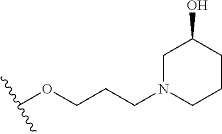 [SMILES: *OCCCN1CCC[C@H](O)C1] | 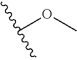 [SMILES: *OC] | 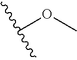 [SMILES: *OC] | 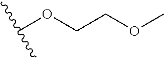 [SMILES: *OCCOC] |
| 55 | 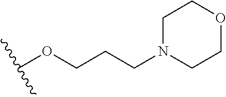 [SMILES: *OCCCN1CCOCC1] | 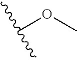 [SMILES: *OC] | 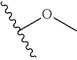 [SMILES: *OC] | 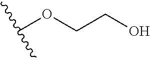 [SMILES: *OCCO] |
| 56 | 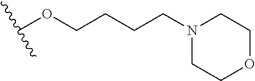 [SMILES: *OCCCCN1CCOCC1] | 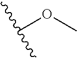 [SMILES: *OC] | 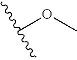 [SMILES: *OC] | 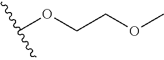 [SMILES: *OCCOC] |
| 57 | 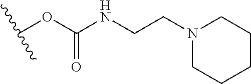 [SMILES: *OC(=O)NCCN1CCCCC1] | 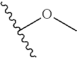 [SMILES: *OC] | 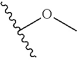 [SMILES: *OC] | 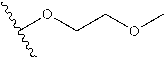 [SMILES: *OCCOC] |
| 58 | 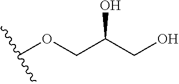 [SMILES: *OC[C@@H](O)CO] | 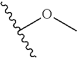 [SMILES: *OC] | 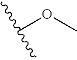 [SMILES: *OC] | 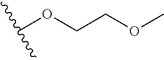 [SMILES: *OCCOC] |
| 59 | 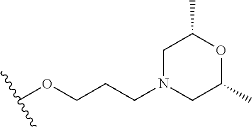 [SMILES: *OCCCN1C[C@@H](C)O[C@@H](C)C1] | 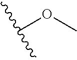 [SMILES: *OC] | 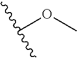 [SMILES: *OC] | 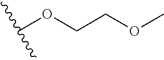 [SMILES: *OCCOC] |
| 60 | 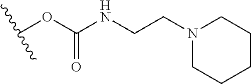 [SMILES: *OC(=O)NCCN1CCCCC1] | 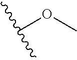 [SMILES: *OC] | 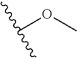 [SMILES: *OC] | 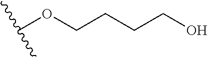 [SMILES: *OCCCCO] |
| 61 | 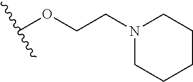 [SMILES: *OCCN1CCCCC1] | 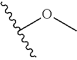 [SMILES: *OC] | 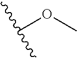 [SMILES: *OC] | 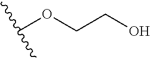 [SMILES: *OCCO] |
| 62 | 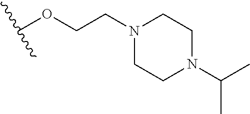 [SMILES: *OCCN1CCN(C(C)C)CC1] | 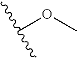 [SMILES: *OC] | 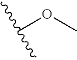 [SMILES: *OC] | 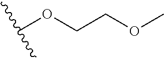 [SMILES: *OCCOC] |
| 63 | 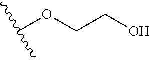 [SMILES: *OCCO] | 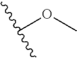 [SMILES: *OC] | 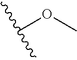 [SMILES: *OC] | 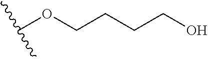 [SMILES: *OCCCCO] |
| 64 | 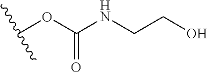 [SMILES: *OC(=O)NCCO] | 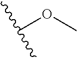 [SMILES: *OC] | 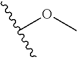 [SMILES: *OC] | 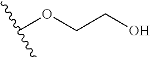 [SMILES: *OCCO] |
| 65 | 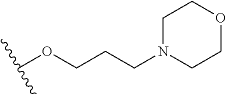 [SMILES: *OCCCN1CCOCC1] | 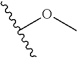 [SMILES: *OC] | 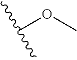 [SMILES: *OC] | 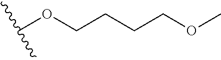 [SMILES: *OCCCCOC] |
| 66 | 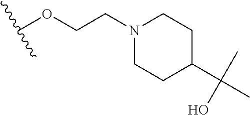 [SMILES: *OCCN1CCC(C(C)(C)O)CC1] | 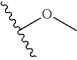 [SMILES: *OC] | 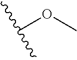 [SMILES: *OC] | 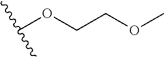 [SMILES: *OCCOC] |
| 67 | 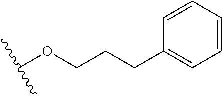 [SMILES: *OCCCc1ccccc1] | 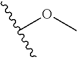 [SMILES: *OC] | 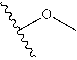 [SMILES: *OC] | 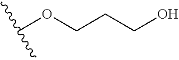 [SMILES: *OCCCO] |
| 68 | 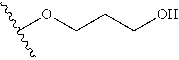 [SMILES: *OCCCO] | 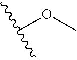 [SMILES: *OC] | 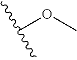 [SMILES: *OC] | 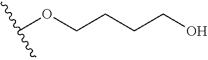 [SMILES: *OCCCCO] |
| 69 | 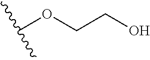 [SMILES: *OCCO] | 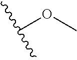 [SMILES: *OC] | 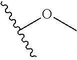 [SMILES: *OC] | 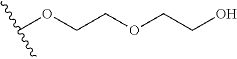 [SMILES: *OCCOCCO] |
| 70 | 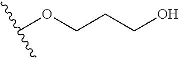 [SMILES: *OCCCO] | 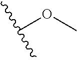 [SMILES: *OC] | 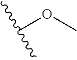 [SMILES: *OC] | 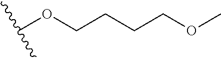 [SMILES: *OCCCCOC] |
| 71 | 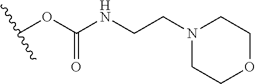 [SMILES: *OC(=O)NCCN1CCOCC1] | 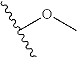 [SMILES: *OC] | 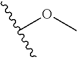 [SMILES: *OC] | 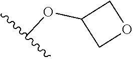 [SMILES: *OC1COC1] |
| 72 | 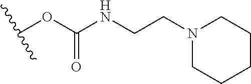 [SMILES: *OC(=O)NCCN1CCCCC1] | 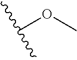 [SMILES: *OC] | 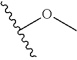 [SMILES: *OC] | 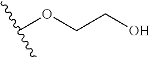 [SMILES: *OCCO] |
| 73 | 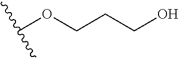 [SMILES: *OCCCO] | 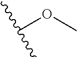 [SMILES: *OC] | 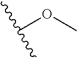 [SMILES: *OC] | 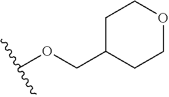 [SMILES: *OCC1CCOCC1] |
| 74 | 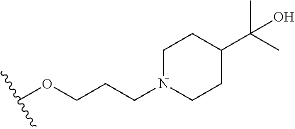 [SMILES: *OCCCN1CCC(C(C)(C)O)CC1] | 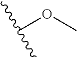 [SMILES: *OC] | 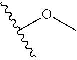 [SMILES: *OC] | 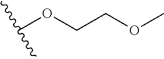 [SMILES: *OCCOC] |
| 75 | 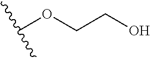 [SMILES: *OCCO] | 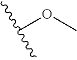 [SMILES: *OC] | 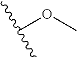 [SMILES: *OC] | 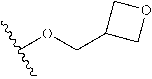 [SMILES: *OCC1COC1] |
| 76 | 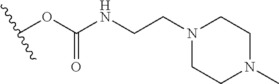 [SMILES: *OC(=O)NCCN1CCN(C)CC1] | 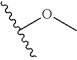 [SMILES: *OC] | 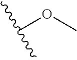 [SMILES: *OC] | 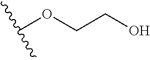 [SMILES: *OCCO] |
| 77 | 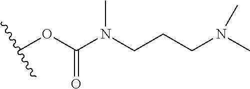 [SMILES: *OC(=O)N(C)CCCN(C)C] | 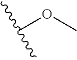 [SMILES: *OC] | 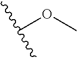 [SMILES: *OC] | 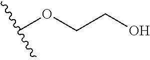 [SMILES: *OCCO] |
| 78 | 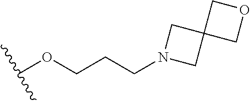 [SMILES: *OCCCN1CC2(COC2)C1] | 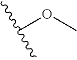 [SMILES: *OC] | 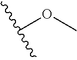 [SMILES: *OC] | 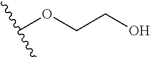 [SMILES: *OCCO] |
| 79 | 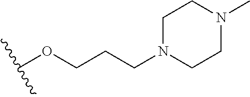 [SMILES: *OCCCN1CCN(C)CC1] | 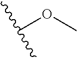 [SMILES: *OC] | 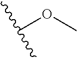 [SMILES: *OC] | 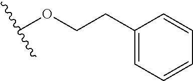 [SMILES: *OCCc1ccccc1] |
| 80 | 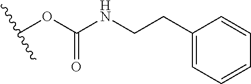 [SMILES: *OC(=O)NCCc1ccccc1] | 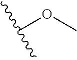 [SMILES: *OC] | 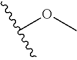 [SMILES: *OC] | 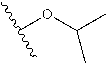 [SMILES: *OC(C)C] |
| 81 | 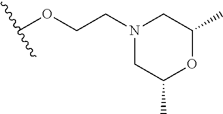 [SMILES: *OCCN1C[C@@H](C)O[C@@H](C)C1] | 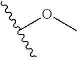 [SMILES: *OC] | 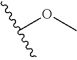 [SMILES: *OC] | 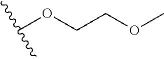 [SMILES: *OCCOC] |
| 82 | 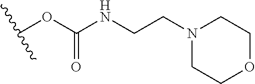 [SMILES: *OC(=O)NCCN1CCOCC1] | 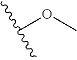 [SMILES: *OC] | 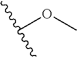 [SMILES: *OC] | 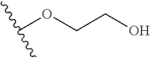 [SMILES: *OCCO] |
| 83 | 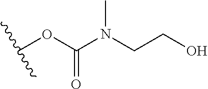 [SMILES: *OC(=O)N(C)CCO] | 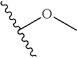 [SMILES: *OC] | 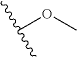 [SMILES: *OC] | 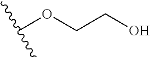 [SMILES: *OCCO] |
| 84 | 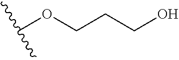 [SMILES: *OCCCO] | 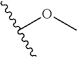 [SMILES: *OC] | 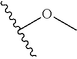 [SMILES: *OC] | 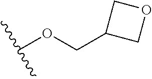 [SMILES: *OCC1COC1] |
| 85 | 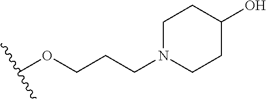 [SMILES: *OCCCN1CCC(O)CC1] | 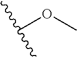 [SMILES: *OC] | 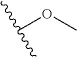 [SMILES: *OC] | 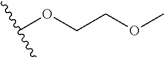 [SMILES: *OCCOC] |
| 86 | 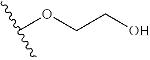 [SMILES: *OCCO] | 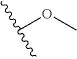 [SMILES: *OC] | 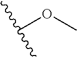 [SMILES: *OC] | 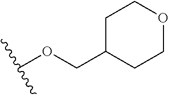 [SMILES: *OCC1CCOCC1] |
| 87 | 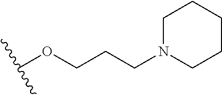 [SMILES: *OCCCN1CCCCC1] | 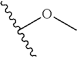 [SMILES: *OC] | 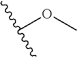 [SMILES: *OC] | 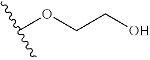 [SMILES: *OCCO] |
| 88 | 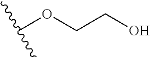 [SMILES: *OCCO] | 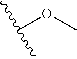 [SMILES: *OC] | 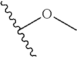 [SMILES: *OC] | 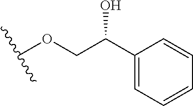 [SMILES: *OC[C@H](O)c1ccccc1] |
| 89 | 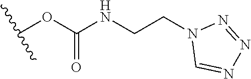 [SMILES: *OC(=O)NCCn1cnnn1] | 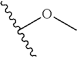 [SMILES: *OC] | 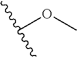 [SMILES: *OC] | 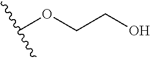 [SMILES: *OCCO] |
| 90 | 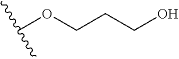 [SMILES: *OCCCO] | 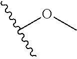 [SMILES: *OC] | 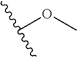 [SMILES: *OC] | 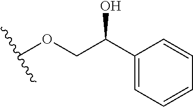 [SMILES: *OC[C@@H](O)c1ccccc1] |
| 91 | 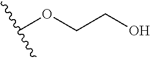 [SMILES: *OCCO] | 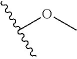 [SMILES: *OC] | 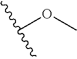 [SMILES: *OC] | 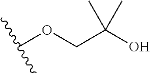 [SMILES: *OCC(C)(C)O] |
| 92 | 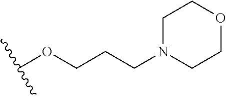 [SMILES: *OCCCN1CCOCC1] | 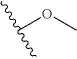 [SMILES: *OC] | 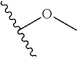 [SMILES: *OC] | 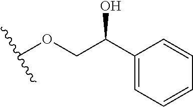 [SMILES: *OC[C@@H](O)c1ccccc1] |
| 93 | 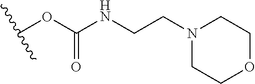 [SMILES: *OC(=O)NCCN1CCOCC1] | 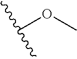 [SMILES: *OC] | 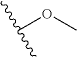 [SMILES: *OC] | 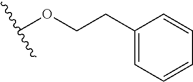 [SMILES: *OCCc1ccccc1] |
| 94 | 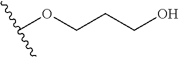 [SMILES: *OCCCO] | 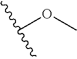 [SMILES: *OC] | 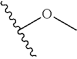 [SMILES: *OC] | 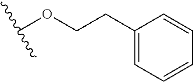 [SMILES: *OCCc1ccccc1] |
| 95 | 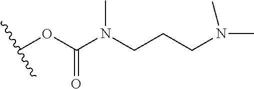 [SMILES: *OC(=O)N(C)CCCN(C)C] | 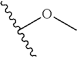 [SMILES: *OC] | 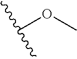 [SMILES: *OC] | 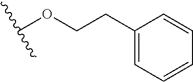 [SMILES: *OCCc1ccccc1] |
| 96 | 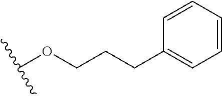 [SMILES: *OCCCc1ccccc1] | 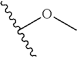 [SMILES: *OC] | 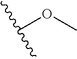 [SMILES: *OC] | 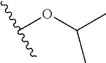 [SMILES: *OC(C)C] |
| 97 | 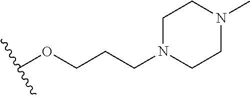 [SMILES: *OCCCN1CCN(C)CC1] | 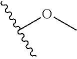 [SMILES: *OC] | 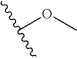 [SMILES: *OC] | 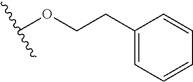 [SMILES: *OCCc1ccccc1] |
| 98 | 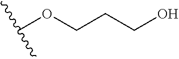 [SMILES: *OCCCO] | 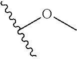 [SMILES: *OC] | 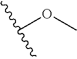 [SMILES: *OC] | 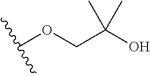 [SMILES: *OCC(C)(C)O] |
| 99 | 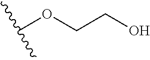 [SMILES: *OCCO] | 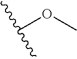 [SMILES: *OC] | 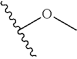 [SMILES: *OC] | 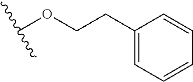 [SMILES: *OCCc1ccccc1] |
| 100 | 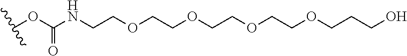 [SMILES: *OC(=O)NCCOCCOCCOCCOCCCO] | 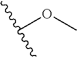 [SMILES: *OC] | 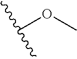 [SMILES: *OC] | 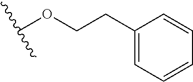 [SMILES: *OCCc1ccccc1] |
| 101 | 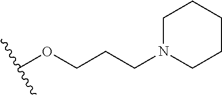 [SMILES: *OCCCN1CCCCC1] | 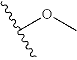 [SMILES: *OC] | 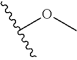 [SMILES: *OC] | 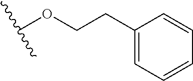 [SMILES: *OCCc1ccccc1] |
| 102 | 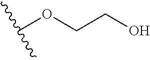 [SMILES: *OCCO] | 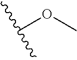 [SMILES: *OC] | 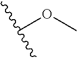 [SMILES: *OC] | 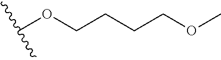 [SMILES: *OCCCCOC] |
| 103 | 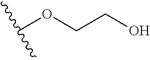 [SMILES: *OCCO] | 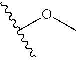 [SMILES: *OC] | 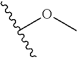 [SMILES: *OC] | 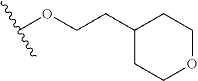 [SMILES: *OCCC1CCOCC1] |
| 104 | 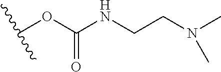 [SMILES: *OC(=O)NCCN(C)C] | 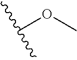 [SMILES: *OC] | 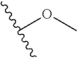 [SMILES: *OC] | 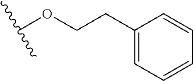 [SMILES: *OCCc1ccccc1] |
| 105 | 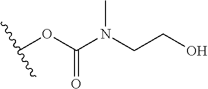 [SMILES: *OC(=O)N(C)CCO] | 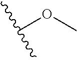 [SMILES: *OC] | 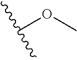 [SMILES: *OC] | 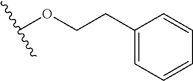 [SMILES: *OCCc1ccccc1] |
| 106 | 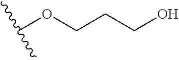 [SMILES: *OCCCO] | 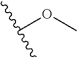 [SMILES: *OC] | 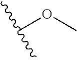 [SMILES: *OC] | 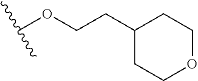 [SMILES: *OCCC1CCOCC1] |
| 107 | 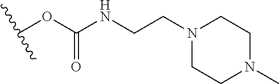 [SMILES: *OC(=O)NCCN1CCN(C)CC1] | 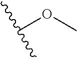 [SMILES: *OC] | 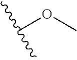 [SMILES: *OC] | 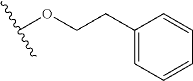 [SMILES: *OCCc1ccccc1] |
| 108 | 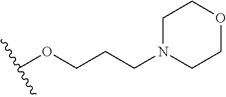 [SMILES: *OCCCN1CCOCC1] | 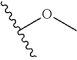 [SMILES: *OC] | 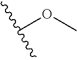 [SMILES: *OC] | 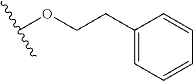 [SMILES: *OCCc1ccccc1] |
| 109 | 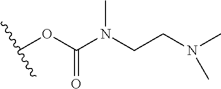 [SMILES: *OC(=O)N(C)CCN(C)C] | 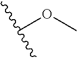 [SMILES: *OC] | 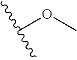 [SMILES: *OC] | 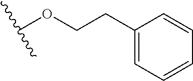 [SMILES: *OCCc1ccccc1] |
| 110 | 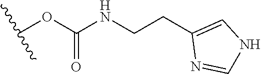 [SMILES: *OC(=O)NCCc1c[nH]cn1] | 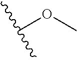 [SMILES: *OC] | 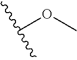 [SMILES: *OC] | 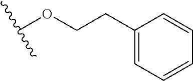 [SMILES: *OCCc1ccccc1] |
| 111 | 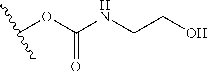 [SMILES: *OC(=O)NCCO] | 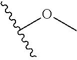 [SMILES: *OC] | 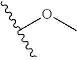 [SMILES: *OC] | 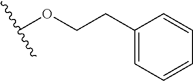 [SMILES: *OCCc1ccccc1] |
| 112 | 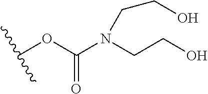 [SMILES: *OC(=O)N(CCO)CCO] | 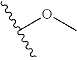 [SMILES: *OC] | 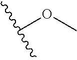 [SMILES: *OC] | 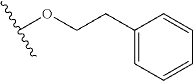 [SMILES: *OCCc1ccccc1] |
| 113 | 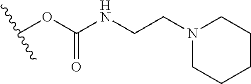 [SMILES: *OC(=O)NCCN1CCCCC1] | 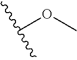 [SMILES: *OC] | 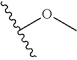 [SMILES: *OC] | 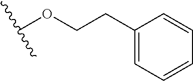 [SMILES: *OCCc1ccccc1] |
| 114 | 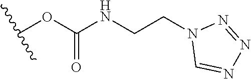 [SMILES: *OC(=O)NCCn1cnnn1] | 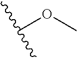 [SMILES: *OC] | 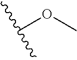 [SMILES: *OC] | 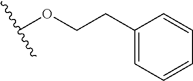 [SMILES: *OCCc1ccccc1] |
| 115 | 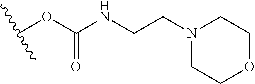 [SMILES: *OC(=O)NCCN1CCOCC1] | 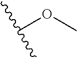 [SMILES: *OC] | 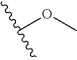 [SMILES: *OC] | 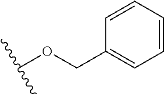 [SMILES: *OCc1ccccc1] |
| 116 | 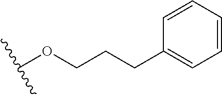 [SMILES: *OCCCc1ccccc1] | 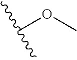 [SMILES: *OC] | 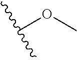 [SMILES: *OC] | 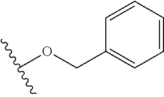 [SMILES: *OCc1ccccc1] |
| 117 | 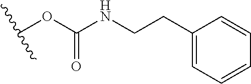 [SMILES: *OC(=O)NCCc1ccccc1] | 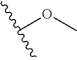 [SMILES: *OC] | 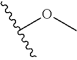 [SMILES: *OC] | 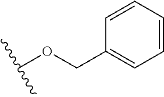 [SMILES: *OCc1ccccc1] |
| 118 | 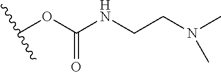 [SMILES: *OC(=O)NCCN(C)C] | 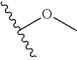 [SMILES: *OC] | 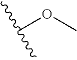 [SMILES: *OC] | 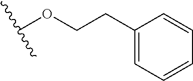 [SMILES: *OCCc1ccccc1] |
| 119 | 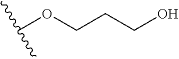 [SMILES: *OCCCO] | 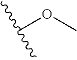 [SMILES: *OC] | 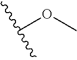 [SMILES: *OC] | 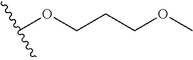 [SMILES: *OCCCOC] |

In some embodiments, the present invention provides a compound of Formula I or I′, wherein the compound is not one or more of the compounds in Table 1A. In some embodiments, the present invention provides a compound of Formula I or I′ excluding the compounds of Table 1A, or pharmaceutically acceptable salt, stereoisomers, tautomers, and polymorphs thereof, wherein one or more hydrogens were replaced by deuterium.

In some embodiments, the present invention provides a compound of Formula I or I′, or pharmaceutically acceptable salt, stereoisomers, tautomers, and polymorphs thereof, wherein when:

### L² is

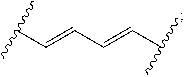 [SMILES: */C=C/C=C/*]

R³ and R^{3′} are hydrogen;
R⁵ and R^{5′} are taken together to form ═O;
and X¹ and X² are both —CH₂—; then:
—X-L¹-R¹, —X³—R², R⁴, and R⁶ are present in a combination other than those combinations presented in below in each row of Table 1A.

### 4. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably inhibit mTORC1, in a biological sample or in a patient. In certain embodiments, the amount of compound in compositions of this invention is such that is effective to measurably inhibit mTORC1, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are inhibitors of mTORC1 and are therefore useful for treating one or more disorders associated with activity of mTORC1. Thus, in certain embodiments, the present invention provides a method for treating an mTORC1-mediated disorder comprising the step of administering to a patient in need thereof a compound of the present invention, or pharmaceutically acceptable composition thereof.

As used herein, the terms “mTORC1-mediated” disorders, diseases, and/or conditions as used herein means any disease or other deleterious condition in which mTORC1, is known to play a role. Accordingly, another embodiment of the present invention relates to treating or lessening the severity of one or more diseases in which mTORC1 is known to play a role. In certain embodiments, an mTORC1-mediated disorder, disease, and/or condition is selected from those described by Matt Kaeberlin, Scientifica, vol. 2013, Article ID 849186.

The methods described herein include methods for the treatment of cancer in a subject. As used in this context, to “treat” means to ameliorate or improve at least one symptom or clinical parameter of the cancer. For example, a treatment can result in a reduction in tumor size or growth rate. A treatment need not cure the cancer or cause remission 100% of the time, in all subjects.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancer cells.

Cancers that can be treated or diagnoses using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

In some embodiments, the methods described herein are used for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

In some embodiments, the cancers that are treated by the methods described herein are cancers that have increased levels of mTORC1 or an increased expression or activity of a mTORC1 relative to normal tissues or to other cancers of the same tissues; methods known in the art and described herein can be used to identify those cancers. In some embodiments, the methods include obtaining a sample comprising cells of the cancer, determining the mTORC1 activity in the sample, and administering a treatment as described herein (e.g., a provided inhibitor of mTORC1). In some embodiments, the cancer is one that is shown herein to have increased levels of mTORC1 activity.

In some embodiments, the present invention provides a method for treating one or more disorders, diseases, and/or conditions wherein the disorder, disease, or condition includes, but is not limited to, a cellular proliferative disorder.

### Cellular Proliferative Disorders

The present invention features methods and compositions for the diagnosis and prognosis of cellular proliferative disorders (e.g., cancer) and the treatment of these disorders by inhibiting mTORC1 activity. Cellular proliferative disorders described herein include, e.g., cancer, obesity, and proliferation-dependent diseases. Such disorders may be diagnosed using methods known in the art.

### Cancer

Cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). In some embodiments, the cancer is melanoma or breast cancer.

### Fibrotic Diseases

Idiopathic Pulmonary Fibrosis (IPF). The PI3K pathway is activated in fibrotic foci, the cardinal lesions in IPF. mTOR kinase inhibitor GSK2126458 reduces PI3K pathway signaling and functional responses in IPF-derived lung fibroblasts and mTOR inhibition reduces collagen expression in models of IPF patients. In the bleomycin model of pulmonary fibrosis, rapamycin treatment is antifibrotic, and rapamycin also decreases expression of α-smooth muscle actin and fibronectin by fibroblasts in vitro.

In some embodiments, the method of inhibiting mTORC1 activity is used to treat idiopathic pulmonary fibrosis (IPF) (see Mercer, P. F. et al., Thorax., 71(8): 701-11 (2016); Patel, A. S., et al., PLoS One, 7(7): e41394 (2012)) Accordingly, in some embodiments, the present invention provides a method of treating idiopathic pulmonary fibrosis (IPF), in a patient in need thereof, comprising the step of administering to said patient a provided compound or pharmaceutically acceptable salt thereof.

Kidney Fibrosis. mTORC1 is activated in myofibroblasts, a major pathogenic cell type in kidney fibrosis. Inhibition of mTOR with rapamycin in a murine model of kidney fibrosis (UUO), attenuated expression of markers of fibrosis and tubulointerstitial damage.

In some embodiments, the method of inhibiting mTORC1 activity is used to treat kidney fibrosis (see Jiang, L., et al., J Am Soc Nephrol, 24(7): 1114-26 (2013); Wu, M. J. et al., Kidney International, 69(11): 2029-36 (2006); Chen, G. et al., PLoS One, 7(3): e33626 (2012); Liu, C. F. et al., Clin Invest Med, 37(34): E142-53 (2014)). Accordingly, in some embodiments, the present invention provides a method of treating kidney fibrosis, in a patient in need thereof, comprising the step of administering to said patient a provided compound or pharmaceutically acceptable salt thereof.

In some embodiments, the method of inhibiting mTORC1 activity is used to treat scleroderma (see Mitra, A., et al., J Invest Dermatol. 135(11): 2873-6 (2015)). Accordingly, in some embodiments, the present invention provides a method of treating scleroderma, in a patient in need thereof, comprising the step of administering to said patient a provided compound or pharmaceutically acceptable salt thereof.

In some embodiments, the method of inhibiting mTORC1 activity is used to treat hypertrophic scarring and keloid disease (see Syed, F., et al., Am J Pathol. 181(5): 1642-58 (2012)). Accordingly, in some embodiments, the present invention provides a method of treating hypertrophic scarring and keloid disease, in a patient in need thereof, comprising the step of administering to said patient a provided compound or pharmaceutically acceptable salt thereof.

In some embodiments, the method of inhibiting mTORC1 activity is used to treat cardiac fibrosis (see Yano, T., et al., J Mol Cell Cardiol. 91: 6-9 (2016)). Accordingly, in some embodiments, the present invention provides a method of treating cardiac fibrosis, in a patient in need thereof, comprising the step of administering to said patient a provided compound or pharmaceutically acceptable salt thereof.

### Other Proliferative Diseases

Other proliferative diseases include, e.g., obesity, benign prostatic hyperplasia, psoriasis, abnormal keratinization, lymphoproliferative disorders (e.g., a disorder in which there is abnormal proliferation of cells of the lymphatic system), chronic rheumatoid arthritis, arteriosclerosis, restenosis, and diabetic retinopathy. Proliferative diseases that are hereby incorporated by reference include those described in U.S. Pat. Nos. 5,639,600 and 7,087,648.

### Other Disorders

Other disorders include lysosomal storage diseases, including, but not limited to, Pompe disease, Gaucher disease, mucopolysaccharidosis, multiple sulfatase deficiency; neurodegenerative diseases such as Parkinson's disease, Alzheimer's disease, Huntington's disease, alpha1-anti-trypsin deficiency, and spinal bulbar muscular atrophy.

In some embodiments, the method of inhibiting mTORC1 activity is used to treat asthma (see Hua, W., et al., Respirology, 20(7): 1055-65 (2015)). Accordingly, in some embodiments, the present invention provides a method of treating asthma, in a patient in need thereof, comprising the step of administering to said patient a provided compound or pharmaceutically acceptable salt thereof.

In some embodiments, the method of inhibiting mTORC1 activity is used to treat a lysosomal storage disease (see Sardiello, M., Annals of the New York Academy of Sciences, 1371(1): 3-14 (2016); Awad, O., et al., Hum Mol Genet. 24(20): 5775-88 (2015); Spampanato, C., et al., EMBO Mol Med., 5(5): 691-706 (2013); Medina, D. L., et al., Dev Cell., 21(3): 421-30 (2011)). Accordingly, in some embodiments, the present invention provides a method of treating a lysosomal storage disease, in a patient in need thereof, comprising the step of administering to said patient a provided compound or pharmaceutically acceptable salt thereof.

In some embodiments, the method of inhibiting mTORC1 activity is used to treat Parkinson's disease (see Decressac, M., et al., Proc Natl Acad Sci USA., 110(19):E1817-26 (2013)). Accordingly, in some embodiments, the present invention provides a method of treating Parkinson's disease, in a patient in need thereof, comprising the step of administering to said patient a provided compound or pharmaceutically acceptable salt thereof.

In some embodiments, the method of inhibiting mTORC1 activity is used to treat Alzheimer's disease (see Polito, V. A., et al., EMBO Mol Med. 6(9):1142-60 (2014)). Accordingly, in some embodiments, the present invention provides a method of treating Alzheimer's disease, in a patient in need thereof, comprising the step of administering to said patient a provided compound or pharmaceutically acceptable salt thereof.

In some embodiments, the method of inhibiting mTORC1 activity is used to treat Huntington's disease (see Tsunemi, T., et al., Sci Transl Med., 4(142): 142ra97 (2012)). Accordingly, in some embodiments, the present invention provides a method of treating Huntington's disease, in a patient in need thereof, comprising the step of administering to said patient a provided compound or pharmaceutically acceptable salt thereof.

In some embodiments, the method of inhibiting mTORC1 activity is used to treat alpha-1-anti-trypsin deficiency (see Pastore, N. et al., EMBO Mol Med., 5(3): 397-412 (2013)). Accordingly, in some embodiments, the present invention provides a method of treating alpha1-anti-trypsin deficiency, in a patient in need thereof, comprising the step of administering to said patient a provided compound or pharmaceutically acceptable salt thereof.

In some embodiments, the method of inhibiting mTORC1 activity is used to treat spinal bulbar muscular atrophy (see Cortes, C. J., et al., Nat Neurosci., 17(9): 1180-9 (2014)). Accordingly, in some embodiments, the present invention provides a method of treating spinal bulbar muscular atrophy, in a patient in need thereof, comprising the step of administering to said patient a provided compound or pharmaceutically acceptable salt thereof.

In some embodiment, the method of inhibiting mTORC1 activity is used to treat Fragile X syndrome (FXS), amyotrophic lateral sclerosis (ALS), epilepsy, focal cortical dysplasia (FCD), hemimegalencephaly (HME), familial focal epilepsy with variable foci (FFEV), temporal lobe epilepsy (TLE), seizures, neurodegenerative diseases, Down syndrome, Rett syndrome (RTS), or diseases associated with activation or hyperactivation of mTOR signaling in the brain.

In some embodiments, the present invention provides a method of treating Fragile X syndrome (FXS) in a patient in need thereof, comprising administering a compound of the present invention, or a pharmaceutically salt thereof.

In some embodiments, the present invention provides a method of treating amyotrophic lateral sclerosis (ALS) in a patient in need thereof, comprising administering a compound of the present invention, or a pharmaceutically salt thereof.

In some embodiments, the present invention provides a method of treating epilepsy in a patient in need thereof, comprising administering a compound of the present invention, or a pharmaceutically salt thereof.

In some embodiments, the present invention provides a method of treating focal cortical dysplasia (FCD) in a patient in need thereof, comprising administering a compound of the present invention, or a pharmaceutically salt thereof.

In some embodiments, the present invention provides a method of treating hemimegalencephaly (HME) in a patient in need thereof, comprising administering a compound of the present invention, or a pharmaceutically salt thereof.

In some embodiments, the present invention provides a method of treating familial focal epilepsy with variable foci (FFEV) in a patient in need thereof, comprising administering a compound of the present invention, or a pharmaceutically salt thereof.

In some embodiments, the present invention provides a method of treating temporal lobe epilepsy (TLE) in a patient in need thereof, comprising administering a compound of the present invention, or a pharmaceutically salt thereof.

In some embodiments, the present invention provides a method of treating seizures in a patient in need thereof, comprising administering a compound of the present invention, or a pharmaceutically salt thereof.

In some embodiments, the present invention provides a method of treating neurodegenerative diseases in a patient in need thereof, comprising administering a compound of the present invention, or a pharmaceutically salt thereof.

In some embodiments, the present invention provides a method of treating Down syndrome in a patient in need thereof, comprising administering a compound of the present invention, or a pharmaceutically salt thereof.

In some embodiments, the present invention provides a method of treating Rett syndrome (RTS) in a patient in need thereof, comprising administering a compound of the present invention, or a pharmaceutically salt thereof.

In some embodiments, the present invention provides a method of treating diseases associated with activation or hyperactivation of mTOR signaling in the brain in a patient in need thereof, comprising administering a compound of the present invention, or a pharmaceutically salt thereof.

In some embodiments, a compound of the present invention binds to FKBP12 to form a complex. In some embodiments, the complex between a compound of the present invention and FKBP12 interacts with the FK506-rapamycin binding domain of mTOR.

In some embodiments, a compound of the present invention binds FKBP12 and interferes with protein-protein interaction between FRAP and FKBP12. In some embodiments, the R¹ group of a compound of the present invention interacts with both FRAP and FKBP12.

The present invention provides compounds that are inhibitors of mTORC1 activity and were shown to selectively inhibit mTORC1 over mTORC2 as measured by pS6K inhibition (a measure of mTORC1 activity) and pAKT activation (a measure of mTORC2 activity). In some embodiments, a provided compound inhibits mTORC1 selectively over mTORC2. In some embodiments, a provided compound does not measurably inhibit mTORC2. In some embodiments, a provided compound has a pAKT activation IC₅₀ of >10 μM. In some embodiments, a provided compound inhibits mTORC1 with >10-fold selectivity over mTORC2. In some embodiments, a provided compound inhibits mTORC1 with >20-fold selectivity over mTORC2. In some embodiments, a provided compound inhibits mTORC1 with >50-fold selectivity over mTORC2. In some embodiments, a provided compound inhibits mTORC1 with >100-fold selectivity over mTORC2. In some embodiments, a provided compound inhibits mTORC1 with >150-fold selectivity over mTORC2. In some embodiments, a provided compound inhibits mTORC1 with >200-fold selectivity over mTORC2. In some embodiments, a provided compound inhibits mTORC1 with >500-fold selectivity over mTORC2. In some embodiments, a provided compound inhibits mTORC1 with >1,000-fold selectivity over mTORC2.

In some embodiments, a provided compound inhibits mTORC1 selectively over mTORC2 after chronic treatment or exposure. In some embodiments, a provided compound inhibits mTORC1 selectively over mTORC2 after about 24 hours of treatment or exposure. In some embodiments, a provided compound inhibits mTORC1 selectively over mTORC2 after about 36 hours of treatment or exposure. In some embodiments, a provided compound inhibits mTORC1 selectively over mTORC2 after about 48 hours of treatment or exposure. In some embodiments, a provided compound inhibits mTORC1 selectively over mTORC2 after about 72 hours of treatment or exposure. In some embodiments, a provided compound inhibits mTORC1 selectively over mTORC2 after about 96 hours of treatment or exposure. In some embodiments, a provided compound inhibits mTORC1 selectively over mTORC2 after about 120 hours of treatment or exposure. In some embodiments, a provided compound inhibits mTORC1 selectively over mTORC2 after about 144 hours of treatment or exposure. In some embodiments, a provided compound inhibits mTORC1 selectively over mTORC2 after about one week of treatment or exposure. In some embodiments, a provided compound inhibits mTORC1 selectively over mTORC2 after more than about one week of treatment or exposure.

In some embodiments, a provided compound is less immunosuppressive than existing rapalogs. In some embodiments, a provided compound is less immunosuppressive than rapamycin. In some embodiments, a provided compound is less immunosuppressive than everolimus. In some embodiments, a provided compound is less immunosuppressive than temsirolimus. In some embodiments, a provided compound is less immunosuppressive than ridaforolimus. In some embodiments, a provided compound is less immunosuppressive than umirolimus.

In some embodiments, a provided compound suppresses interferon gamma (IFN-γ) production less than rapalogs. In some embodiments, a provided compound suppresses IFN-γ production less than rapamycin. In some embodiments, a provided compound suppresses IFN-γ production less than everolimus. In some embodiments, a provided compound suppresses IFN-γ production less than temsirolimus. In some embodiments, a provided compound suppresses IFN-γ production less than ridaforolimus. In some embodiments, a provided compound suppresses IFN-γ production less than umirolimus.

In some embodiments, a provided compound decreases the expression of fibrosis biomarkers in tissue that has been damaged. In some embodiments, a provided compound decreases the expression of collagen I (COL1A2) in tissue that has been damaged. In some embodiments, a provided compound decreases the expression of collagen III (COL3A1) in tissue that has been damaged. In some embodiments, a provided compound decreases the expression of fibronectin (FN1) in tissue that has been damaged.

In some embodiments, a provided compound decreases the propensity of immune cells from infiltrating damaged tissue. In some embodiments, a provided compound decreases the propensity of macrophage cells from infiltrating damaged tissue.

In some embodiments, a provided compound induces less glucose tolerance than rapalogs. In some embodiments, a provided compound induces less glucose tolerance than rapamycin. In some embodiments, a provided compound induces less glucose tolerance than everolimus. In some embodiments, a provided compound induces less glucose tolerance than temsirolimus. In some embodiments, a provided compound induces less glucose tolerance than ridaforolimus. In some embodiments, a provided compound induces less glucose tolerance than umirolimus. In some embodiments, a provided compound does not induce glucose tolerance significantly more than a placebo or vehicle alone.

Accordingly, in some embodiments, the present invention provides a method of treating a disorder associate with mTORC1 comprising administering to patient a compound that inhibits mTORC1 wherein said compound does not inhibit mTORC2. Such compounds may be employed for indications where rapamycin and rapalogs demonstrated a benefit either in animal models or in a human disease setting. Such indications include:

Treatment of Metabolic Disease (Obesity and Insulin Resistance in Type 2 Diabetes). Inhibition of mTORC1 pathway leads to extension of life span in yeast, fly and mouse, and caloric restriction improves longevity and insulin sensitivity. The underlying mechanism has been proposed to function by regulation of mTORC1 activation. Rapamycin-induced insulin resistance has been shown to be mediated by inhibition of mTORC2 and selective mTORC1 inhibitor is predicted to improve insulin sensitivity and glucose homeostasis.

In some embodiments, the method of inhibiting mTORC1 activity is used to treat metabolic disease (obesity and insulin resistance in type 2 diabetes) (see Yu, Z., et al., J Gerontol A Biol Sci Med Sci, 70(4), 410-20 (2015); Fok, W. C., et al., Aging Cell 13 (2): 311-9 (2014); Shum, M., et al., Diabetologia, 59(3):592-603 (2016); Lamming, D. W., et al., Science 335(6076): 1638-43 (2012)). Accordingly, in some embodiments, the present invention provides a method of treating metabolic disease (obesity and insulin resistance in type 2 diabetes), in a patient in need thereof, comprising the step of administering to said patient a provided compound or pharmaceutically acceptable salt thereof.

Neurofibromatosis. Neurofibromatosis type 1 (NF1) is caused by mutations in the NF1 gene. Its protein product, neurofibromin, functions as a tumor suppressor and ultimately produces constitutive upregulation of mTOR. mTOR inhibitors have been shown to reduce tumor size and induce anti-proliferative effect in NF1-associated plexiform neurofibroma.

In some embodiments, the method of inhibiting mTORC1 activity is used to treat neurofibromatosis (see Franz, D. N., et al., Curr Neurol Neurosci Rep., 12(3): 294-301 (2012); Varin, J., et al., Oncotarget., 7: 35753-67 (2016)). Accordingly, in some embodiments, the present invention provides a method of treating neurofibromatosis, in a patient in need thereof, comprising the step of administering to said patient a provided compound or pharmaceutically acceptable salt thereof.

Cardiomyopathy and skeletal muscle dystrophy, Emery-Dreifuss muscular dystrophy model (LMNA^{-/-}). Mutations in LMNA result in several human diseases including limb-girdle muscular dystrophy (LGMD1B), Emery-Dreifuss muscular dystrophy (EDMD2/3), dilated cardiomyopathy (DCM) and conduction-system disease (CMD1A), lipodystrophy, Charcot-Marie-Tooth disease, and Hutchinson-Gilford progeria syndrome (HGPS). Lmna^{-/-} mice have elevated mTORC1 activity and short-term treatment with rapamycin in Lmna^{-/-} mice results in reduced mTORC1 signaling, improved cardiac and skeletal muscle function and enhanced survival by ˜50%.

In some embodiments, the method of inhibiting mTORC1 activity is used to treat cardiomyopathy and skeletal muscle dystrophy (see Ramos, F., et al., Sci Transl Med., 4(144): 144ra103 (2012); Bonne, G. & Quijano-Roy, S., Handb Clin Neurol., 113: 1367-76 (2013)). Accordingly, in some embodiments, the present invention provides a method of treating cardiomyopathy and skeletal muscle dystrophy, in a patient in need thereof, comprising the step of administering to said patient a provided compound or pharmaceutically acceptable salt thereof.

Leigh syndrome. Ndufs4 knockout (KO) mice are used as a model of Leigh syndrome and exhibit hyperactivation of mTORC1 and metabolic defects. Treatment of Ndufs4 KO mice with rapamycin extended lifespan, improve metabolic and neurological defect associated with this disease.

In some embodiments, the method of inhibiting mTORC1 activity is used to treat Leigh syndrome (see Johnson, S. C., et al., Science, 342(6165): 1524-8 (2013)). Accordingly, in some embodiments, the present invention provides a method of treating Leigh syndrome, in a patient in need thereof, comprising the step of administering to said patient a provided compound or pharmaceutically acceptable salt thereof.

Oncology. Inhibition of mTOR with rapalogs has been shown to have antitumor activity in murine cancer models and in cancer patients. Examples of sensitive cancer types include, but are not limited to, hepatocellular carcinoma, breast cancers, mantle cell lymphomas, lung carcinoma, tuberous sclerosis and lymphangioleiomyomatosis.

In some embodiments, the method of inhibiting mTORC1 activity is used to treat cancer and oncologic disorders (see Ilagan, E. & manning, B. D., Trends Cancer, 2(5): 241-51 (2016)). Accordingly, in some embodiments, the present invention provides a method of treating cancer and oncologic disorders, in a patient in need thereof, comprising the step of administering to said patient a provided compound or pharmaceutically acceptable salt thereof.

Non-alcoholic steatohepatitis (NASH). The present invention provides inhibitors that induce autophagy to clear degraded cytoplasmic proteins, and NASH disease is characterized by lipid deposits, inflammation and fibrosis in the liver. The inhibition of mTORC1 pathway induce autophagy and down regulate SREBP-1 to decrease lipid biosynthesis to reduce lipid storage.

In some embodiments, the method of inhibiting mTORC1 activity is used to treat non-alcoholic steatohepatitis (NASH) (see Puri, P. & Chandra, A., J Clin Exp Hepatol, 4(1): 51-9 (2014)). Accordingly, in some embodiments, the present invention provides a method of treating non-alcoholic steatohepatitis (NASH), in a patient in need thereof, comprising the step of administering to said patient a provided compound or pharmaceutically acceptable salt thereof.

Tuberous sclerosis (TSC) and lymphangioleiomyomatosis (LAM). Failure in the regulation of mTOR is critical to the pathogenesis of the inherited disorder tuberous sclerosis complex (TSC) and the related lung disease, lymphangioleiomyomatosis (LAM). Both diseases are caused by mutations of TSC1 or TSC2 leading to inappropriate activity of signaling downstream of mTORC1. TSC patients develop nonmalignant tumors in many organs, including the brain, while LAM patients, mostly women, accumulate abnormal, muscle-like cells in certain organs or tissues, especially the lungs, lymph nodes, and kidneys. The rapalogs, everolimus and sirolimus, are currently approved for the treatment of both TSC and LAM, respectively, by the U.S. FDA.

In some embodiments, the method of inhibiting mTORC1 activity is used to treat tuberous sclerosis and lymphangioleiomyomatosis (see Wander, S. A., et al., J. Clin. Invest., 121(4): 1231-41 (2011); Taveira-DaSilva, A. M. & Moss, J., J. Clin Epidemiol., 7: 249-57 (2015)). Accordingly, in some embodiments, the present invention provides a method of treating tuberous sclerosis and lymphangioleiomyomatosis, in a patient in need thereof, comprising the step of administering to said patient a provided compound or pharmaceutically acceptable salt thereof.

Senescence and diseases of aging. Rapamycin suppresses the mammalian TORC1 complex, which regulates translation, and extends lifespan in diverse species, including mice. Rapamycin was shown to inhibit the pro-inflammatory phenotype of senescent cells. As senescent cells accumulate with age, the senescence-associated secretory phenotype (SASP) can disrupt tissues and contribute to age-related pathologies, including cancer. Inhibition of mTOR suppressed the secretion of inflammatory cytokines by senescent cells. Rapamycin reduced cytokine levels including IL6 and suppressed translation of the membrane-bound cytokine IL1A. Reduced IL1A diminishes NF-κB transcriptional activity, which controls the SASP. Thus, mTORC1 inhibitors might ameliorate age-related pathologies, including late-life cancer, by suppressing senescence-associated inflammation.

In some embodiments, the method of inhibiting mTORC1 activity is used to treat senescence and diseases of aging (see Laberge, R. M., et al., Nature Cell Biology, 17(8): 1049-61 (2015); Nacarelli, T., et al., Free Radic Biol Med., 95: 133-54 (2016)). Accordingly, in some embodiments, the present invention provides a method of treating senescence and diseases of aging, in a patient in need thereof, comprising the step of administering to said patient a provided compound or pharmaceutically acceptable salt thereof.

Diabetic nephropathy and kidney-related complications of type 1 diabetes and type 2 diabetes. Diabetic nephropathy is a kidney complication of type-1 and type-2 diabetes, affecting up to nearly 40% of people with diabetes. High levels of glucose force the kidneys work excessively to filter blood, resulting in kidney damage. Studies suggest that the mTOR pathway is highly activated in patients with diabetic neuropathy and may play a role in the pathological changes and renal dysfunction due to chronic high glucose. Further, mTOR inhibition may attenuate hyperinsulinemia.

In some embodiments, the method of inhibiting mTORC1 activity is used to treat diabetic nephropathy or kidney-related complications of type 1 diabetes and type 2 diabetes (see Mori, H., et al., Biochem. Res. Commun. 384(4): 471-5 (2009)). Accordingly, in some embodiments, the present invention provides a method of treating diabetic nephropathy or kidney-related complications of type 1 diabetes and type 2 diabetes in a patient in need thereof, comprising the step of administering to said patient a provided compound or pharmaceutically acceptable salt thereof.

Polycystic kidney disease. Polycystic kidney disease (PKD) is characterized by the development and accumulation of destructive kidney cysts that eventually result in kidney failure. PKD may be autosomal dominant (ADPKD) or recessive (ARPKD). Dysfunctional mTOR signaling pathway has been observed in ADPKD and ARPKD. Thus, normalization of the mTORC1 pathway may ameliorate the development of cysts and progression of the disease.

In some embodiments, the method of inhibiting mTORC1 activity is used to treat PKD (see Torres, V. E., et al., Clin. J. Am. Soc. Nephrol. 5(7): 1312-29 (2010)). Accordingly, in some embodiments, the present invention provides a method of treating PKD in a patient in need thereof, comprising the step of administering to said patient a provided compound or pharmaceutically acceptable salt thereof. In some embodiments, PKD is autosomal dominate. In some embodiments, PKD is autosomal recessive.

Focal Segmental Glomerulosclerosis (FSGS) and other diseases associated with sclerosis of the kidney. FSGS is the most common primary glomerular disorder causing end-stage renal disease (ESRD) in the United States. As the disease progresses there is a mismatch of podocyte cells in Bowman's capsule and the surface area of the glomerular basement membrane they cover. Studies have shown that podocyte size control is regulated by mTOR and that mTOR activation contributes to disease progression. Further, constitutive mTORC1 activation has been shown to cause FSGS-like lesions in mouse knockdown experiments. Thus, mTORC1 inhibition might ameliorate (FSGS) or other diseases associated with sclerosis of the kidney by normalizing or increasing autophagic activity.

In some embodiments, the method of inhibiting mTORC1 activity is used to treat FSGS or other diseases associated with sclerosis of the kidney (see Zschiedrich, S. et al., J. Am. Soc. Nephrol. 28(7): 2144-57 (2017)). Accordingly, in some embodiments, the present invention provides a method of treating FSGS or other diseases associated with sclerosis of the kidney in a patient in need thereof, comprising the step of administering to said patient a provided compound or pharmaceutically acceptable salt thereof.

Age-Related Macular Degeneration. Age-related macular degeneration (AMD) is a leading cause of blindness characterized by the death of photoreceptors in the macula. Possible mechanisms of AMD progression include oxidative stress leading to deposits of proteins and dysfunctional organelles, leading to retinal pigment epithelium hypertrophy, dedifferentiation, and eventual atrophy. mTOR is implicated in the dedifferentiation of the retinal pigment epithelium. Thus, mTORC1 inhibition may ameliorate AMD by blocking hypertrophy and dedifferentiation.

In some embodiments, the method of inhibiting mTORC1 activity is used to treat age-related macular degeneration (see Kolosova, N. G., et al., Am. J. Path. 181(2): 472-7 (2012) and Zhen, C. & Vollrath, D., Aging 3(4): 346-47 (2011)). Accordingly, in some embodiments, the present invention provides a method of treating age-related macular degeneration in a patient in need thereof, comprising the step of administering to said patient a provided compound or pharmaceutically acceptable salt thereof.

Diabetic Macular Edema. Diabetic macular edema (DME) is a leading cause of blindness in persons with diabetes, affecting approximately 35% of people with diabetes. Studies suggest that the pathogenesis of DME is an inflammatory disease involving various cytokines and chemokines. Chronic inflammatory and oxidative stress may contribute to the progression of DME. Thus, inhibition of mTORC1 may ameliorate DME symptoms and progression by decreasing the inflammatory response.

In some embodiments, the method of inhibiting mTORC1 activity is used to treat DME (see Okamoto, T., et al., PLOS ONE, (11)(1): e0146517, https://doi.org/10.1371/journal.pone.0146517 (2016)). Accordingly, in some embodiments, the present invention provides a method of treating DME in a patient in need thereof, comprising the step of administering to said patient a provided compound or pharmaceutically acceptable salt thereof.

Diabetic retinopathy. Diabetic retinopathy (DR) is a common eye disease accounting for ˜5% of blindness in adults and is associated with chronic hyperglycemia and defects of insulin signaling pathways. DR patients suffer persistent injury to retinal blood vessels and neurons by inflammation, reactive oxygen species and endoplasmic reticulum stress caused by chronic hyperglycemia. Significantly, rapamycin has been shown to block the action of insulin-induced hypoxia-inducible factor-1 (HIF-1) and retinal cell senescence, and induces autophagy, and could be beneficial in promoting apoptosis of nascent blood vessels and preventing angiogenesis. Thus, inhibition of mTORC1 may ameliorate DR symptoms and progression by decreasing inflammation and inhibiting pathogenic signaling pathways.

In some embodiments, the method of inhibiting mTORC1 activity is used to treat DR (see Di Rosa, M., et al., Curr. Neuropharmacol. 14(8): 810-25 (2016)). Accordingly, in some embodiments, the present invention provides a method of treating DR in a patient in need thereof, comprising the step of administering to said patient a provided compound or pharmaceutically acceptable salt thereof.

Glaucoma. Glaucoma is a common optic neuropathy associated with aging and elevated intraocular pressure, and is the leading cause of irreversible blindness. Studies suggest that mTOR dependent dysregulation of autophagocytosis may be a factor in the progression of the disease. Thus, inhibition of mTORC1 may slow the progression or ameliorate glaucoma by normalizing or increasing autophagy.

In some embodiments, the method of inhibiting mTORC1 activity is used to treat glaucoma (see Porter, K., et al., Biochim. Biophys. Acta. 1852(3): 379-85 (2014)). Accordingly, in some embodiments, the present invention provides a method of treating glaucoma in a patient in need thereof, comprising the step of administering to said patient a provided compound or pharmaceutically acceptable salt thereof.

Restoring immune function. mTORC1 inhibition has been shown to reduce the expression of programmed death-1 (PD-1) receptor in CD4⁺ and CD8⁺ T lymphocytes, promoting T-cell signaling. Thus, mTORC1 inhibition may restore immune function by improving the adaptive immune response.

In some embodiments, the method of inhibiting mTORC1 activity is used to restore immune function (see Mannick, J. B., et al., Sci. Trans. Med. 6(268): ppra179 (2014)). Accordingly, in some embodiments, the present invention provides a method of restoring immune function in a patient in need thereof, comprising the step of administering to said patient a provided compound or pharmaceutically acceptable salt thereof.

Treatment of respiratory and/or urinary tract infections. mTORC1 inhibition may reduce infections by upregulation of antiviral gene expression and response. Thus, mTORC1 inhibition may enhance the ability of a patient's immune system to defend against respiratory and/or urinary tract infections.

In some embodiments, the method of inhibiting mTORC1 activity is used to treat respiratory and/or urinary tract infections. (see Mannick, J.B., et al., Sci. Trans. Med. 10(449): eaaq1564 (2018)). Accordingly, in some embodiments, the present invention provides a method of restoring immune function in a patient in need thereof, comprising the step of administering to said patient a provided compound or pharmaceutically acceptable salt thereof.

Heart failure. mTORC1 activity is essential for cardiac hypertrophy in response to stress but can lead to cardiac derangements as a result of cardiac remodeling following infarction. Inhibition of mTORC1 reduces cardiac remodeling and heart failure in response to pressure overload. Thus, inhibition of mTORC1 may decrease heart failure in patients who have suffered damage to the myocardium.

In some embodiments, the method of inhibiting mTORC1 activity is used to treat heart failure (see Sciarretta, S. et al., Circ. Res. 122(3): 489-505 (2018)). Accordingly, in some embodiments, the present invention provides a method of treating heart failure in a patient in need thereof, comprising the step of administering to said patient a provided compound or pharmaceutically acceptable salt thereof.

Osteoarthritis. Osteoarthritis (OA) is a chronic degenerative disease resulting in loss of cartilage and joint inflammation. mTOR may play a significant role in collagen homeostasis and turnover and remodeling of cartilage. Thus, inhibition of mTORC1 may slow the progression or ameliorate osteoarthritis symptoms by normalizing cartilage turnover.

In some embodiments, the method of inhibiting mTORC1 activity is used to treat osteoarthritis (see Pal, B., et al., Drugs R&D, 15(1): 27-36 (2017))). Accordingly, in some embodiments, the present invention provides a method of treating osteoarthritis in a patient in need thereof, comprising the step of administering to said patient a provided compound or pharmaceutically acceptable salt thereof.

Pulmonary arterial hypertension. Pulmonary arterial hypertension (PAH) is a progressive, fatal disease associated with increases pulmonary vascular resistance. Pulmonary arterial smooth muscle cell proliferation and migration are implicated in the progressing of arterial wall thickening, exacerbating vasoconstriction. Thus, inhibition of mTORC1 may alleviate PAH by reducing vascular remodeling.

In some embodiments, the method of inhibiting mTORC1 activity is used to treat PAH (see Ma, X., et al., Interact. Cardiovasc. Thorac. Surg. 25(2): 206-11 (2017)). Accordingly, in some embodiments, the present invention provides a method of treating PAH is a patient in need thereof, comprising the step of administering to said patient a provided compound or pharmaceutically acceptable salt thereof.

Chronic Obstructive Pulmonary Disease. Reduced autophagy results in the accumulation of proteins and other cellular materials that accelerate cellular senescence in patients with chronic obstructive pulmonary disease (COPD). Thus, inhibition of mTORC1 may slow the progression or ameliorate COPD symptoms by normalizing or increasing autophagy.

In some embodiments, the method of inhibiting mTORC1 activity is used to treat COPD (see Fujii, S., et al., Oncoimmunology 1(5): 630-41 (2012)). Accordingly, in some embodiments, the present invention provides a method of treating COPD in a patient in need thereof, comprising the step of administering to said patient a provided compound or pharmaceutically acceptable salt thereof.

Additional therapeutic indications where mTORC inhibition may be beneficial are: cardiovascular disease (acute coronary syndrome), coronary occlusions with eluting stents, polycystic kidney disease, and kidney disease associated with cyst formation or cystogenesis), neurofibromatosis, epilepsy assoc. with TSC1 and/or TSC2 mutations, polycystic liver, pachyonychia congenital, fragile x syndrome, Friedrich ataxia, Peutz-Jeghers syndrome, eye disease including neovascular age-related macular degeneration, uveitis, diabetic macular edema, fibroblast growth including pulmonary fibrosis, renal insufficiency/fibrosis, metabolic syndrome, diseases of the immune system including immune senescence, lupus nephritis, chronic immune thrombocytopenia, multiple sclerosis, cancer including lymphoma, tumors associated with TSC1/2 mutations, angiomyolipoma assoc. with TSC1/2 mutations, breast cancer, hepatocellular cancer, leukemia, glioma, adenoid cystic carcinoma, senescence, autism, and vascular rheumatoid arthritis.

In some embodiments, the method of inhibiting mTORC1 activity is used to treat cardiovascular disease (acute coronary syndrome), coronary occlusions with eluting stents, polycystic kidney disease, neurofibromatosis, epilepsy assoc. with TSC1 and/or TSC2 mutations, polycystic liver, pachyonychia congenital, fragile x syndrome, Friedrich ataxia, Peutz-Jeghers syndrome, eye disease including neovascular age-related macular degeneration, uveitis, diabetic macular edema, fibroblast growth including pulmonary fibrosis, renal insufficiency/fibrosis, metabolic syndrome, diseases of the immune system including immune senescence, lupus nephritis, chronic immune thrombocytopenia, multiple sclerosis, cancer including lymphoma, tumors associated with TSC1/2 mutations, angiomyolipoma associated with TSC1/2 mutations, breast cancer, hepatocellular cancer, leukemia, glioma, adenoid cystic carcinoma, senescence, autism, and vascular rheumatoid arthritis.

Accordingly, in some embodiments, the present invention provides a method of treating cardiovascular disease (acute coronary syndrome), coronary occlusions with eluting stents, polycystic kidney disease, neurofibromatosis, epilepsy assoc. with TSC1 and/or TSC2 mutations, polycystic liver, pachyonychia congenital, fragile x syndrome, Friedrich ataxia, Peutz-Jeghers syndrome, eye disease including neovascular age-related macular degeneration, uveitis, diabetic macular edema, fibroblast growth including pulmonary fibrosis, renal insufficiency/fibrosis, metabolic syndrome, diseases of the immune system including immune senescence, lupus nephritis, chronic immune thrombocytopenia, multiple sclerosis, cancer including lymphoma, tumors associated with TSC1/2 mutations, angiomyolipoma assoc. with TSC1/2 mutations, breast cancer, hepatocellular cancer, leukemia, glioma, adenoid cystic carcinoma, senescence, autism, and vascular rheumatoid arthritis, in a patient in need thereof, comprising the step of administering to said patient a provided compound or pharmaceutically acceptable salt thereof.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

In other embodiments, the present invention provides a method for treating a disorder mediated by mTORC1 in a patient in need thereof, comprising the step of administering to said patient a compound according to the present invention or pharmaceutically acceptable composition thereof. Such disorders are described in detail herein.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g., in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g., under the trademark Carboplat™. Oxaliplatin can be administered, e.g., in the form as it is marketed, e.g., under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g., BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR1 ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO 2008/118802), navitoclax (and analogs thereof, see U.S. Pat. No. 7,390,799), NH-1 (Shenayng Pharmaceutical University), obatoclax (and analogs thereof, see WO 2004/106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

Further examples of BTK inhibitory compounds, and conditions treatable by such compounds in combination with compounds of this invention can be found in WO 2008/039218 and WO 2011/090760, the entirety of which are incorporated herein by reference.

Further examples of SYK inhibitory compounds, and conditions treatable by such compounds in combination with compounds of this invention can be found in WO 2003/063794, WO 2005/007623, and WO 2006/078846, the entirety of which are incorporated herein by reference.

Further examples of PI3K inhibitory compounds, and conditions treatable by such compounds in combination with compounds of this invention can be found in WO 2004/019973, WO 2004/089925, WO 2007/016176, U.S. Pat. No. 8,138,347, WO 2002/088112, WO 2007/084786, WO 2007/129161, WO 2006/122806, WO 2005/113554, and WO 2007/044729 the entirety of which are incorporated herein by reference.

Further examples of JAK inhibitory compounds, and conditions treatable by such compounds in combination with compounds of this invention can be found in WO 2009/114512, WO 2008/109943, WO 2007/053452, WO 2000/142246, and WO 2007/070514, the entirety of which are incorporated herein by reference.

Further anti-angiogenic compounds include compounds having another mechanism for their activity, e.g., unrelated to protein or lipid kinase inhibition, e.g., thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are, e.g., inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™, CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g., hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art (see Hellman, Principles of Radiation Therapy, Cancer, in Principles and Practice of Oncology, Devita et al., Eds., 4^{th} Edition, Vol. 1, pp. 248-275 (1993)).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocortisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code numbers, generic or trade names may be taken from the actual edition of the standard compendium “The Merck Index” or from databases, e.g., Patents International (e.g., IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In some embodiments, the additional therapeutic agent administered in combination with a compound of the present invention is another mTOR inhibitor. In some embodiments, the additional mTOR inhibitor inhibits mTOR by binding the catalytic active site of mTOR. Examples of such additional mTOR inhibitors include: dactolisib, 8-(6-methoxy-pyridin-3-yl)-3-methyl-1-(4-piperazin-1-yl-3-trifluoromethyl-phenyl)-1,3-dihydro-imidazo[4,5-c]quinolin-2-one (WO 2006/122806), vistusertib (AZD2014; WO 2009/153597); AZD8055 (WO 2009/153597; XL388 (U.S. Pat. App. Pub. 2010/0305093); sapanisertib (MLN0128; INK128; WO 2015/051043); DS3078; apitolisib (GDC0980; WO 2008/070740); omipalisib (GSK-2126458; WO 2008/14446); NVP-BGT226 (Chang, K. Y., et al., Clin. Cancer Res. 17(22): 7116-26 (2011)); voxtalisib (XL765; SAR245409; WO 2007/044813); PF04691502 (WO 2008/032162); gedatolisib (PF05212384; PKI-587; WO 2009/143313); SF1126 (WO 2004/089925); GSK1059615 (WO 2007/136940); BI-860585; OSI 027 (WO 2007/061737); VS 5584 (WO 2010/114484); CC-223 (WO 2010/062571); DCBCI-0901 (Lee, Y. E., et al., Mol. Canc. Thera. 12(11 Suppl): Abstract nr C270 (2013)):); LY3023414 (WO 2012/097039); P529 (WO 2007/133249); panulisib (P7170; WO 2012/007926); DS-7423 (Kashiyama, T., et al., PLoS One 9(2): e87220 (2014)); PWT33567 mesylate (VCD-597; WO 2010/110685); ME-344 (NV-128; Navarro, P., et al., Cell Rep. 15(12):2705-18 (2016)); ABTL0812 (WO 2010/106211); WYE-132; EXEL-3885 (Eur J Cancer Suppl. 6(12): Abst 322 (2008)); EXEL-4431 (Eur J Cancer Suppl. 6(12): Abst 322 (2008)); AR-mTOR-26 (101st Annu Meet Am Assoc Cancer Res (AACR) (April 17-21, Washington, D.C.) 2010, Abst 4484); NV-128 (A. B. Alvero et al., Mol Cancer Ther. 10(8): 1385-93 (2011)); salinomycin (VS-507; Gupta, P. B., et al., Cell 138(4): 645-59 (2009)); BN-107; BN-108; WAY-600; WYE-687; WYE-354 (Yu, K., et al., Cancer Res. 69(15): 6232-40 (2009)); Ku-063794 (Garcia-Martinez, J. M., et al., Biochem. J. 421(1): 29-42 (2009)); torkinib (PP242; Apsel, B., et al., Nat. Chem. Biol. 4(11): 691-99 (2008)); PP30; CZ415 (REF); INK1069; EXEL-2044; EXEL-7518; SB2158; SB2280; AR-mTOR-1 (Wallace, E. M., et al., Mol. Canc. Thera. 8(12 Suppl): Abst. B267 (2009)).

Reference to any particular additional mTOR inhibitor herein also comprises any pharmaceutically acceptable salts, stereoisomers, tautomers, solvates, hydrates and polymorphs thereof.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein (see also Luengo, J. I. et al., Chem. Biol., 2(7): 471-81 (1995); Grinfeld, A. A. et al., Tet. Lett., 35(37): 6835-38 (1994); PCT/US2019/037507; and PCT/US2020/063351, incorporated herein by reference in their entireties).

Where an Example which follow hereinafter lists only analytical measurements such as LC/MS, ¹H NMR, ¹⁹F NMR, etc. (rather than reaction step details), it will be understood that the title compound was prepared according to the procedures as described in the synthesis schemes and Examples herein, selecting and substituting suitable reagents and reactants, as would be readily recognized by those skilled in the art.

Unless otherwise indicated in the examples, all temperature is expressed in Centigrade (° C.). All reactions were conducted under an inert atmosphere at ambient temperature unless otherwise noted. Unless otherwise specified, reaction solutions were stirred at room temperature under a N₂(g) or Ar(g) atmosphere. Reagents employed without synthetic details are commercially available or made according to known methods, for example according to literature procedures. When solutions were “concentrated to dryness”, they were concentrated using a rotary evaporator under reduced pressure, when solutions were dried, they were typically dried over a drying agent such as MgSO₄ or Na₂SO₄. Where a synthesis product is listed as having been isolated as a residue, it will be understood by those skilled in the art that the term “residue” does not limit the physical state in which the product was isolated and may include, for example, a solid, an oil, a foam, a gum, a syrup, and the like.

In obtaining the compounds described in the examples below and the corresponding analytical data, the following experimental and analytical protocols were followed unless otherwise indicated.

LC-MS: Unless otherwise indicated, the analytical LC-MS system used consisted of a Shimadzu LCMS-2020 with electrospray ionization (ESI) in positive ion detection mode with 20ADXR pump, SIL-20ACXR autosampler, CTO-20AC column oven, M20A PDA Detector and LCMS 2020 MS detector. The column was a HALO a C18 30*5.0 mm, 2.7 μm. The mobile phase A was water containing 0.05% TFA and mobile phase B was acetonitrile containing 0.05% TFA. The gradient was from 5% mobile phase B to 100% (95%) in 2.0 min, hold 0.7 min, then revert to 5% mobile phase B over 0.05 min and maintain for 0.25 min. The Column Oven (CTO-20AC) was operated at a 40.0° C. The flow rate was 1.5 mL/min, and the injection volume was 1 μl. PDA (SPD-M20A) detection was in the range 190-400 nm. The MS detector, which was configured with electrospray ionization as ionizable source; Acquisition mode: Scan; Nebulizing Gas Flow:1.5 L/min; Drying Gas Flow:15 L/min; Detector Voltage: Tuning Voltage±0.2 kv; DL Temperature: 250° C.; Heat Block Temperature: 250° C.; Scan Range: 90.00-900.00 m/z. ELSD (Alltech 3300) detector Parameters: Drift Tube Temperature:60±5° C.; N2 Flow-Rate: 1.8±0.2 L/min. Mobile phase gradients were optimized for the individual compounds. Calculated mass corresponds to the exact mass.

Preparative HPLC: Unless otherwise noted, preparative HPLC purifications were performed with Waters Auto purification system (2545-2767) with a 2489 UV detector. The column was selected from one of the following: Waters C18, 19×150 mm, 5 μm; XBridge Prep OBD C18 Column, 30×150 mm 5 μm; XSelect CSH Prep C18 OBD Column, 5 μm,19*150 mm; XBridge Shield RP18 OBD Column, 30×150 mm, 5 μm; Xselect CSH Fluoro Phenyl, 30×150 mm, 5 μm; or YMC-Actus Triart C18, 30×150 mm, 5 μm. The mobile phases consisted of mixtures of acetonitrile (5-95%) in water containing 0.1% FA or 10 mmol/L NH₄HCO₃. Flow rates were maintained at 25 mL/min, the injection volume was 1200 μL, and the UV detector used two channels 254 nm and 220 nm. Mobile phase gradients were optimized for the individual compounds.

Normal phase flash chromatography: Unless otherwise noted, normal phase flash column chromatography (FCC) was performed on silica gel with pre-packaged silica gel columns (such as RediSep©), using ethyl acetate (EtOAc)/hexanes, ethyl acetate (EtOAc)/Petroleum ether (b.p. 60-90° C.), CH₂Cl₂/MeOH, or CH₂Cl₂/10% 2N NH₃ in MeOH, as eluent.

¹H NMR: Unless otherwise noted, ¹H NMR spectra were acquired using 400 MHz spectrometers (or 500 MHz spectrometers) in DMSO-d₆ or CDCl₃ solutions. The nuclear magnetic resonance (NMR) spectral characteristics refer to chemical shifts (δ) are expressed in parts per million (ppm). Tetramethylsilane (TMS) was used as internal reference in DMSO-d₆ solutions. Coupling constants (J) are reported in hertz (Hz). The nature of the shifts as to multiplicity is reported as s (singlet), d (doublet), t (triplet), q (quartet), dd (double doublet), dt (double triplet), m (multiplet), br (broad).

### List of Abbreviations Used in the Experimental Section

CH₃CN: acetonitrile

DCM: dichloromethane

DMAP: dimethyl aminopyridine

DMF: N,N-dimethylformamide

DMSO: dimethyl sulfoxide

EDCI: 1-ethyl-3-(3-dimethylaminopropyl)carbodiimide

ESI: electrospray ionization

EtOAc: ethyl acetate

Et₂O: diethyl ether

EtOH: ethanol

h: hours

HCl: hydrogen chloride

HF: hydrogen fluoride

HND-8: acidic ion exchange resin (e.g., Amberlyst)

H₂O: water

HPLC: high performance liquid chromatography

MeOH: methanol

min: minutes

MgSO₄: magnesium sulfate

mL: milliliters

mM: millimolar

mmol: millimoles

MS: mass spectrometry

N₂: nitrogen gas

NaHCO₃: sodium bicarbonate

NaOH: sodium hydroxide

Na₂SO₄: sodium sulfate

NH₃: ammonia

NH₄Cl: ammonium chloride

NMR: nuclear magnetic resonance

° C.: degrees Celsius

prep-HPLC: preparative high performance liquid chromatography

PE: petroleum ether

p-TsOH: para toluenesulfonic acid

rt: room temperature

TASF: tris(dimethylamino)sulfonium difluorotrimethylsilicate

TEA: triethylamine

TFA: trifluoracetic acid

THF: tetrahydrofuran SYNTHESIS EXAMPLES: INTERMEDIATES

### Synthesis of Intermediate I

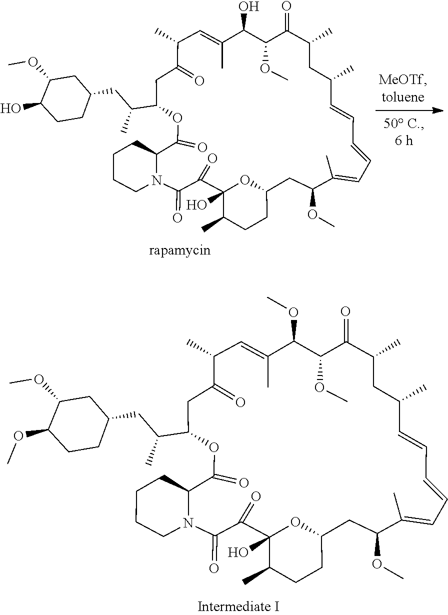 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC)[C@H](OC)C2)CC(=O)[C@H](C)/C=C(\C)[C@@H](OC)[C@@H](OC)C(=O)[C@H](C)C[C@H](C)/C=C/C=C/C=C/1C]

To a solution of rapamycin (0.2 g, 0.22 mmol) in toluene (5 mL) was added proton sponge (0.94 g, 4.38 mmol) at rt, followed by the addition of methyl trifluoromethanesulfonate (0.54 g, 3.28 mmol). The mixture was stirred at 50° C. for 6 h then cooled and purified via silica gel chromatography and reverse phase chromatography (85% CH₃CN in water) to provide (24E,26E,28E,29E,31R,32S,33R,34R,36S,38S,40S,41S,42R,43R,52R)-41-[(1R)-2-[(1S,3R,4R)-3,4-dimethoxycyclohexyl]-1-methyl-ethyl]-52-hydroxy-40,42,43-trimethoxy-31,32,33,34,44,45-hexamethyl-60,61-dioxa-53-azatricyclohexatriaconta-24,26,28(44),29(45)-tetraene-46,47,48,49,50-pentone (50 mg, 24% yield) as a white solid. ESI-MS (EI⁺, m/z): 964.2 [M+Na]⁺. ¹H NMR (400 MHz, CDCl₃) δ 6.50-5.80 (m, 4H), 5.62 (ddd, J=22.9, 14.5, 7.9 Hz, 1H), 5.32 (dt, J=11.6, 7.7 Hz, 2H), 5.18-5.03 (m, 1H), 4.68 (s, 1H), 3.95-3.54 (m, 5H), 3.50-3.33 (m, 7H), 3.32-3.21 (m, 3H), 3.18-2.92 (m, 8H), 2.83-2.48 (m, 3H), 2.25 (dd, J=30.1, 10.7 Hz, 2H), 2.02 (ddd, J=34.0, 26.3, 9.6 Hz, 4H), 1.88-1.56 (m, 14H), 1.51-1.16 (m, 9H), 1.15-0.82 (m, 18H), 0.79-0.68 (m, 1H).

### Synthesis of Intermediate II

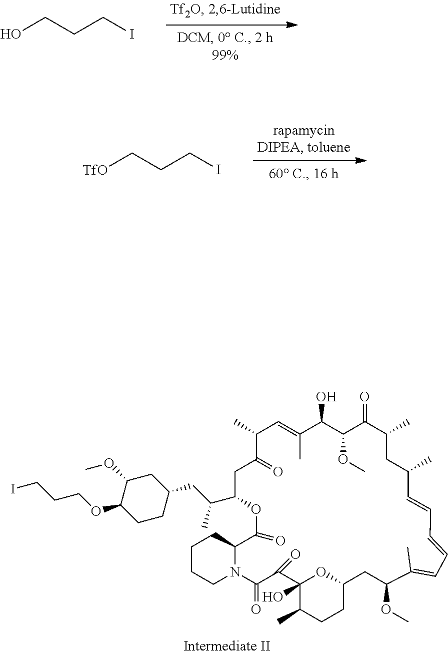 [SMILES: OCCCI ; O=S(=O)(OCCCI)C(F)(F)F ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCI)[C@H](OC)C2)CC(=O)[C@H](C)/C=C(\C)[C@@H](O)[C@@H](OC)C(=O)[C@H](C)C[C@H](C)/C=C/C=C/C=C/1C]

Step 1: 3-iodopropyltrifluoromethanesulfonate. A mixture of 3-iodopropan-1-ol (4 g, 21.51 mmol) and 2,6-lutidine (4.61 g, 43 mmol) in DCM (40 mL) was cooled to 0° C. under N₂ and trifluoromethylsulfonyl trifluoromethanesulfonate (6.67 g, 23.66 mmol) was added dropwise. The resulting solution was stirred at 0° C. for 2 h then quenched with 10% EtOAc in PE and filtered through a short silica gel column. The filtrate was concentrated in vacuo to afford 3-iodopropyl trifluoromethanesulfonate (6.72 g, 98 yield) as a light yellow liquid.

Step 2: (22E,24E,26E,27E,32R,33S,34R,35R,37S,39S,41S,42S,43R,44R,53R)-43,53-dihydroxy-42-[(1R)-2-[(1S,3R,4R)-4-(3-iodopropoxy)-3-methoxy-cyclohexyl]-1-methyl-ethyl]-41,44-dimethoxy-32,33,34,35,45,46-hexamethyl-62,63-dioxa-54-azatricyclohexatriaconta-22,24,26(45),27(46)-tetraene-47,48,49,50,51-pentone (Intermediate II). A mixture of rapamycin (2 g, 2.19 mmol) and N-ethyl-N-isopropyl-propan-2-amine (5.72 mL, 32.82 mmol) in toluene (40 mL) was stirred at 50° C. for 16 h. The mixture was poured onto ice cold saturated aqueous NaHCO₃ (50 mL), washed with ice-water (60 mL×2), brine (50 mL), dried over anhydrous Na₂SO₄, filtered and concentrated. The residue was purified via silica gel chromatography (PE:EA=3:1) to provide Intermediate II (1.45 g, 60% yield) as a light-yellow solid. ESI-MS (EI+, m/z): 1104.5 [M+Na]⁺.

### Synthesis of Intermediates III & VI

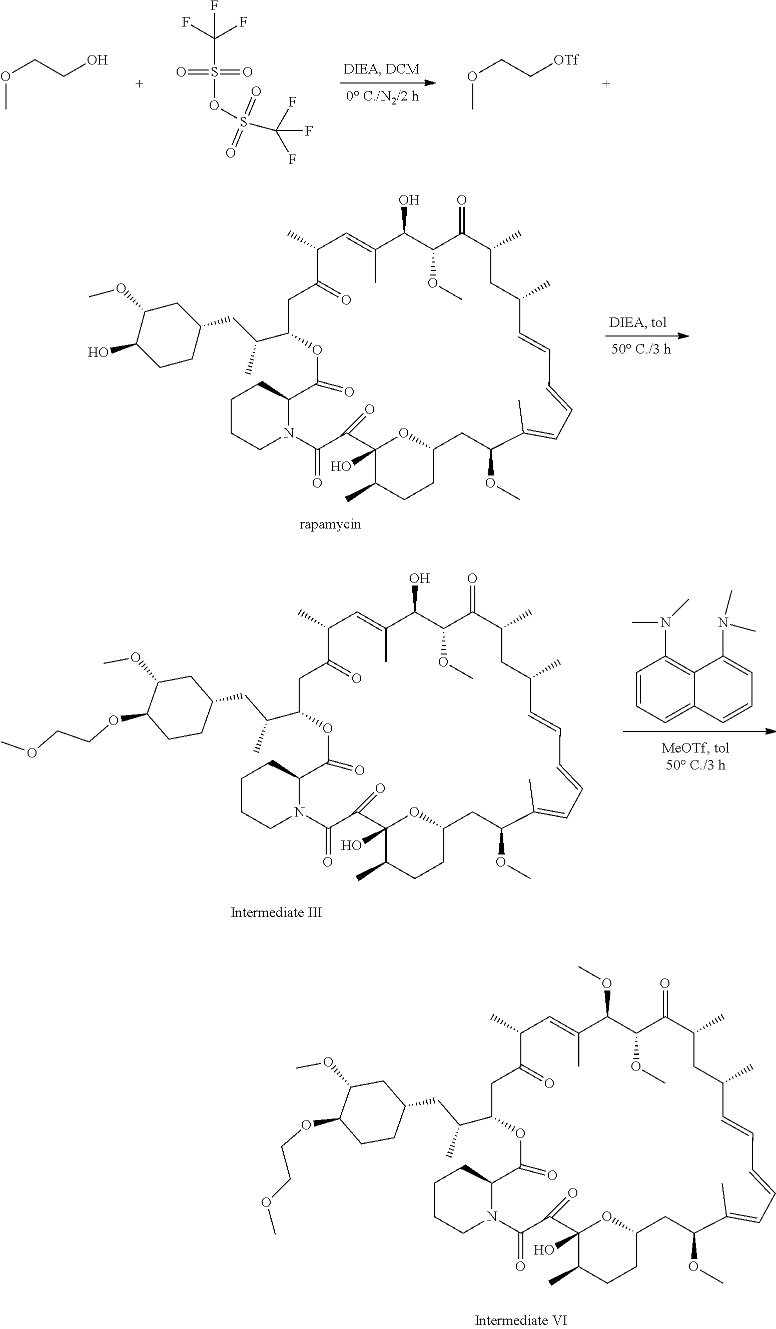

Step 1: 2-methoxyethyl trifluoromethanesulfonate. To a solution of 2-methoxyethanol (4.5 g, 59.14 mmol) and DIEA (11.46 g, 88.71 mmol) in DCM (50 mL) at 0° C. under N₂ was added trifluoromethylsulfonyl trifluoromethanesulfonate (18.35 g, 65.05 mmol) dropwise. This was stirred at 0° C. for 2 h then diluted with DCM (50 mL), washed with Sat.NaHCO₃ (50 mL), water (50 mL), brine (50 mL) then the organic layer was dried over Na₂SO₄, filtered and concentrated in vacuo to afford 2-methoxyethyl trifluoromethanesulfonate (12.3 g, 99% yield) as a brown oil which was used without further purification. ¹H NMR (400 MHz, CDCl₃): δ 4.62 (t, J=4.4 Hz, 2H), 3.71 (t, J=4.6 Hz, 2H), 3.42 (s, 3H).

Step 2: (3S,6R,7E,9R,10R,12R,14S,15E,17E,19E,21S,23S,26R,27R,34aS)-9,27-dihydroxy-10,21-dimethoxy-3-((R)-1-((1S,3R,4R)-3-methoxy-4-(2-methoxyethoxy)cyclohexyl)propan-2-yl)-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (Intermediate III). Rapamycin (3 g, 3.28 mmol) and N-ethyl-N-isopropyl-propan-2-amine (8.48 g, 65.63 mmol) in toluene (30 mL) was stirred at 50° C. for 3 h. The reaction was poured into cold NaHCO₃ (50 mL), washed with ice water (2×60 mL), brine (50 mL) and dried over anhydrous Na₂SO₄, filtered and concentrated. The residue was purified via silica gel chromatography (PE:EA=1:2) to provide Intermediate III (1.2 g, 38% yield) as a white solid. ¹H NMR (400 MHz, CDCl₃): δ 5.95-6.42 (m, 4H), 5.14-5.58 (m, 4H), 4.41-4.81 (m, 1H), 4.17-4.28 (m, 1H), 3.84-4.00 (m, 1H), 3.63-3.79 (m, 4H), 3.49-3.59 (m, 2H), 3.31-3.46 (m, 10H), 3.07-3.22 (m, 5H), 2.55-2.76 (m, 2H), 2.31-2.35 (m, 1H), 1.91-2.10 (m, 3H), 1.61-1.88 (m, 19H), 1.41-1.55 (m, 4H), 1.15-1.36 (m, 7H), 0.83-1.11 (m, 16H), 0.69-0.76 (m, 1H).

Step 3: (3S,6R,7E,9R,10R,12R,14S,15E,17E,19E,21S,23S,26R,27R,34aS)-27-hydroxy-9,10,21-trimethoxy-3-((R)-1-((1S,3R,4R)-3-methoxy-4-(2-methoxyethoxy)cyclohexyl)propan-2-yl)-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (Intermediate VI). To a suspension of (23E,25E,27E,28E,32R,33S,34R,35R,37S,39S,41S,42S,43R,44R,53R)-43,53-dihydroxy-41,44-dimethoxy-42-[(1R)-2-[(1S,3R,4R)-3-methoxy-4-(2-methoxyethoxy)cyclohexyl]-1-methyl-ethyl]-32,33,34,35,45,46-hexamethyl-62,63-dioxa-54-azatricyclohexatriaconta-23,25,27(45),28(46)-tetraene-47,48,49,50,51-pentone (0.5 g, 0.51 mmol) and 1,8-bis(dimethylamino)naphtalene (1.65 g, 7.71 mmol) in toluene (10 mL) was added methyl trifluoromethanesulfonate (1.01 g, 6.17 mmol) dropwise at rt under N₂. The reaction was heated to 50° C. for 3 h then filtered and diluted with EA (60 mL), washed with saturated aqueous NH₄Cl (60 mL×10), water (60 mL) and brine (60 mL). The organic layer was dried over anhydrous sodium sulfate, filtered and concentrated. The residue was purified via silica gel chromatography (PE:EA=1:1) to provide Intermediate VI (92 mg, 18% yield) as a white solid. ¹H NMR (400 MHz, CDCl₃): δ 6.03-6.42 (m, 4H), 5.08-5.60 (m, 4H), 4.10-4.74 (m, 1H), 3.73-3.93 (m, 4H), 3.49-3.71 (m, 5H), 3.44-3.46 (m, 3H), 3.34-3.41 (m, 4H), 3.24-3.31 (m, 3H), 3.07-3.18 (m, 7H), 2.48-2.82 (m, 2H), 1.95-2.35 (m, 5H), 1.53-1.83 (m, 18H), 1.42-1.52 (m, 3H), 1.22-1.37 (m, 6H), 1.04-1.15 (m, 10H), 0.86-0.97 (m, 7H), 0.69-0.79 (m, 1H).

### Synthesis of Intermediate IV

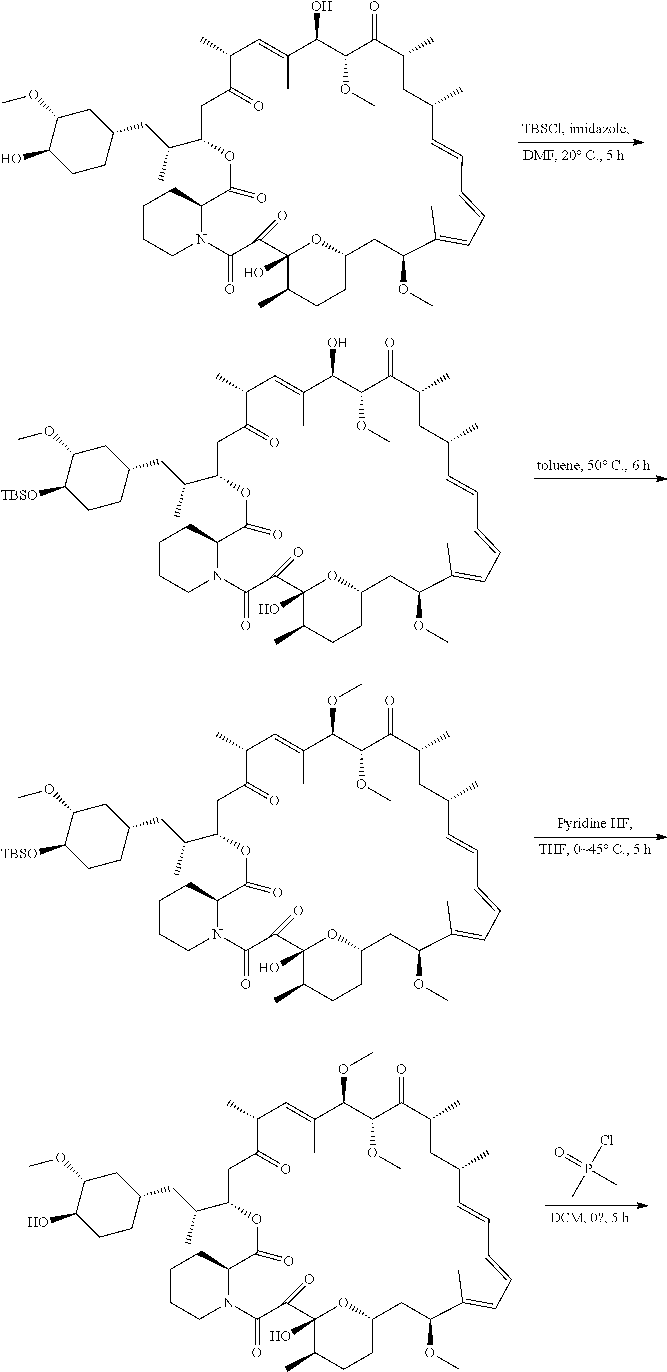

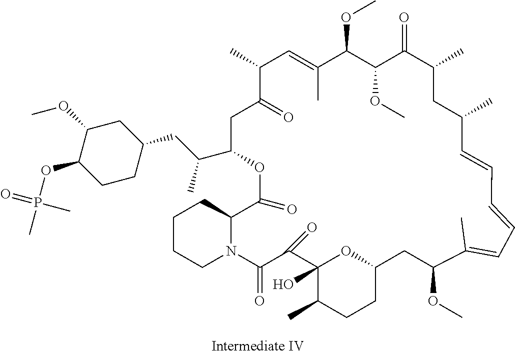 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OP(C)(C)=O)[C@H](OC)C2)CC(=O)[C@H](C)/C=C(\C)[C@@H](OC)[C@@H](OC)C(=O)[C@H](C)C[C@H](C)/C=C/C=C/C=C/1C]

Step 1: (3S,6R,7E,9R,10R,12R,14S,15E,17E,19E,21S,23S,26R,27R,34aS)-3-((R)-1-((1S,3R,4R)-4-((tert-butyldimethylsilyl)oxy)-3-methoxycyclohexyl)propan-2-yl)-9,27-dihydroxy-10,21-dimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone. To a solution of rapamycin (5 g, 5.47 mmol) in DMF (60 mL) was added imidazole (1.49 g, 21.88 mmol) and tert-butyl-chloro-dimethyl-silane (2.47 g, 16.41 mmol, 3.05 mL). The reaction was stirred at 50° C. for 6 h then poured into cold saturated NH₄Cl solution (40 mL) and Et₂O:PE (60 mL, 2:1). The organic layer was washed with saturated NH₄Cl solution (20 mL), water and brine (20 mL), dried over Na₂SO₄, filtered and concentrated. The residue was purified via silica gel chromatography (EtOAc in PE from 10% to 50%) to provide (3S,6R,7E,9R,10R,12R,14S,15E,17E,19E,21S,23S,26R,27R,34aS)-3-((R)-1-((1S,3R,4R)-4-((tert- butyldimethylsilyl)oxy)-3-methoxycyclohexyl)propan-2-yl)-9,27-dihydroxy-10,21-dimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (4 g, 71% yield) as a white solid. ESI-MS (EI⁺, m/z): 1050.5 [M+Na]⁺.

Step 2: (3S,6R,7E,9R,10R,12R,14S,15E,17E,19E,21S,23S,26R,27R,34aS)-3-((R)-1-((1S,3R,4R)-4-((tert-butyldimethylsilyl)oxy)-3-methoxycyclohexyl)propan-2-yl)-27-hydroxy-9,10,21-trimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone. To a suspension of (3S,6R,7E,9R,10R,12R,14S,15E,17E,19E,21S,23S,26R,27R,34aS)-3-((R)-1-((1S,3R,4R)-4-((tert- butyldimethylsilyl)oxy)-3-methoxycyclohexyl)propan-2-yl)-9,27-dihydroxy-10,21-dimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (1 g, 0.97 mmol) and 1,8-bis(dimethylamino)naphtalene (2.5 g, 11.67 mmol) in toluene (15 mL) was added methyl trifluoromethanesulfonate (2.39 g, 14.59 mmol, 1.60 mL) dropwise at rt under N₂. The reaction was then heated to 50° C. for 6 h, cooled and filtered. The filtrate was concentrated and purified via silica gel chromatography to provide (28E,30E,32E,33E,35R,36S,37R,38R,40S,42S,44S,45S,46R,47R,56R)-45-[(1R)-2-[(1S,3R,4R)-4-[tert-butyl(dimethyl)silyl]oxy-3-methoxy-cyclohexyl]-1-methyl-ethyl]-56-hydroxy-44,46,47-trimethoxy-35,36,37,38,48,49-hexamethyl-65,66-dioxa-58-azatricyclohexatriaconta-28,30,32(48),33(49)-tetraene-50,51,52,53,54-pentone (0.45 g, 44% yield) as a white solid. ESI-MS (EI⁺, m/z): 1064.6 [M+Na]⁺.

Step 3: (3S,6R,7E,9R,10R,12R,14S,15E,17E,19E,21S,23S,26R,27R,34aS)-27-hydroxy-3-((R)-1-((1S,3R,4R)-4-hydroxy-3-methoxycyclohexyl)propan-2-yl)-9,10,21-trimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone. To a solution of (3S,6R,7E,9R,10R,12R,14S,15E,17E,19E,21S,23S,26R,27R,34aS)-3-((R)-1-((1S,3R,4R)-4-((tert-butyldimethylsilyl)oxy)-3-methoxycyclohexyl)propan-2-yl)-27-hydroxy-9,10,21-trimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (0.4 g, 0.38 mmol) in THF (10 mL) was added pyridine hydrofluoride (3.8 g, 38.37 mmol) at 0° C. and the mixture stirred at 45° C. for 5 h. The mixture was diluted with DCM and saturated NaHCO₃, washed with water and brine, dried over Na₂SO₄, filtered and concentrated. The residue was purified via reverse phase chromatography to provide (3S,6R,7E,9R,10R,12R,14S,15E,17E,19E,21S,23S,26R,27R,34aS)-27-hydroxy-3-((R)-1-((1S,3R,4R)-4-hydroxy-3-methoxycyclohexyl)propan-2-yl)-9,10,21-trimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (0.16 g, 45% yield) as a white solid. ESI-MS (EI⁺, m/z): 949.9 [M+Na]⁺.

Step 4: (1R,2R,4S)-4-((R)-2-((3S,6R,7E,9R,10R,12R,14S,15E,17E,19E,21S,23S,26R,27R,34aS)-27-hydroxy-9,10,21-trimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)-2-methoxycyclohexyl dimethylphosphinate (Intermediate IV). To a solution of (3S,6R,7E,9R,10R,12R,14S,15E,17E,19E,21S,23S,26R,27R,34aS)-27-hydroxy-3-((R)-1-((1S,3R,4R)-4-hydroxy-3-methoxycyclohexyl)propan-2-yl)-9,10,21-trimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (0.26 g, 0.28 mmol) in DCM (10 mL) was added 2,6-di-tert-butyl-4-methylpyridine (0.17 g, 0.84 mmol) and dimethylphosphinic chloride (0.315 g, 2.80 mmol) in DCM (1 mL) at 0° C. The reaction was stirred at 0° C. for 5 h then diluted with EtOAc, washed with saturated NaHCO₃ solution, ice cold 0.5 N HCl, water, brine, dried over Na₂SO₄, filtered and concentrated. The residue was purified via silica gel chromatography (DCM:MeOH=40:1) to provide Intermediate IV (100 mg, 36% yield) as a white solid. ESI-MS (EI⁺, m/z): 1025.8 [M+Na]⁺.

### Synthesis of Intermediate V/IX

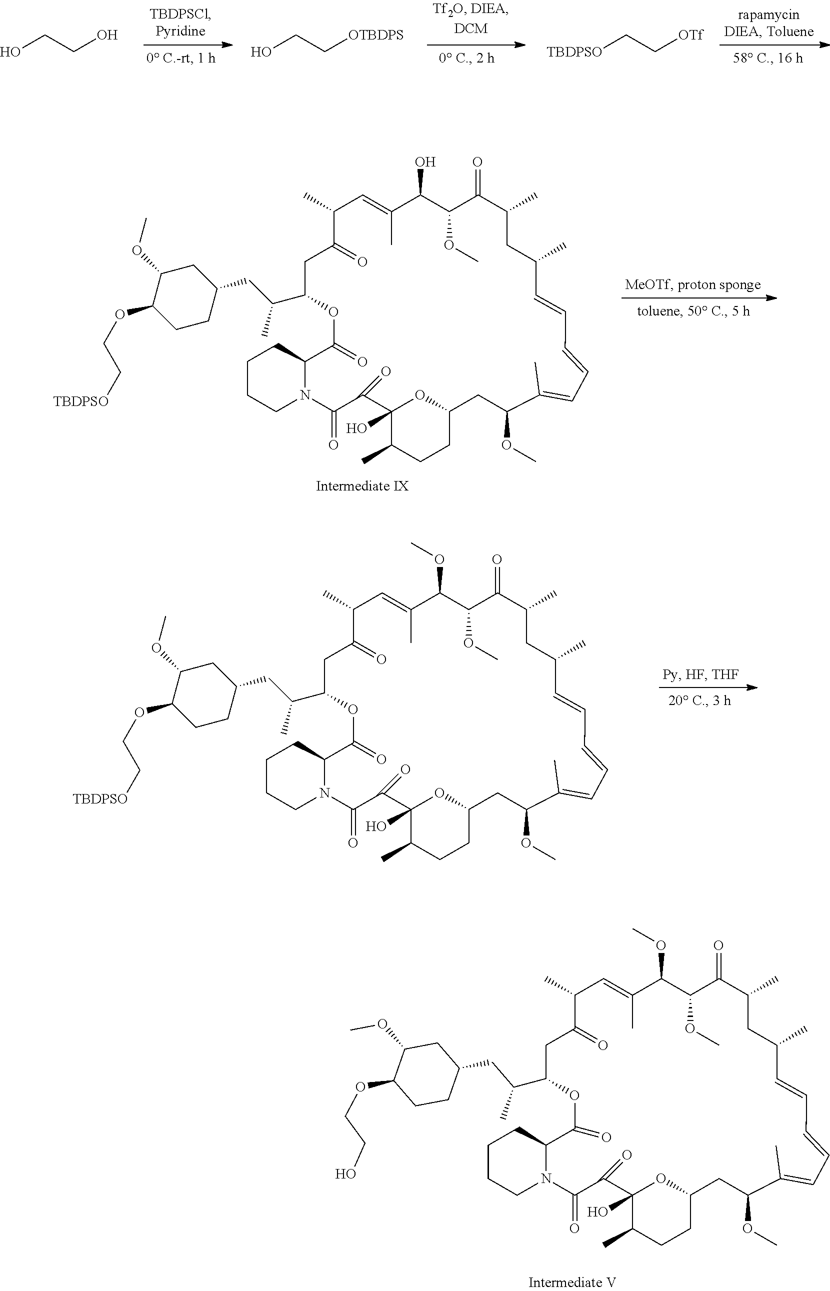

Step 1: 2-((tert-butyldiphenylsilyl) oxy) ethan-1-ol. Tert-butylchlorodiphenylsilane (26.61 g, 96.83 mmol) was added to a solution of ethylene glycol (49.28 g, 793.97 mmol) in pyridine (44 mL) at 0° C. The resulting solution was stirred at rt for 1 h, then poured into water (500 mL) and extracted with EtOAc (200 mL×3). The organic layer was dried over Na₂SO₄, filtered and concentrated. The residue was purified via silica gel chromatography (EA:PE=1:8) to provide 2-((tert-butyldiphenylsilyl)oxy)ethan-1-ol (25 g, 86% yield) as a colorless solid. ESI-MS (EI⁺, m/z): 323.1 [M+Na]⁺.

Step 2: 2-((tert-butyldiphenylsilyl) oxy) ethyl trifluoromethanesulfonate. To a solution of 2-((tert-butyldiphenylsilyl)oxy)ethan-1-ol (17.13 g, 57 mmol) and DIEA (11.05 g, 85.52 mmol) in DCM (120 mL) at 0° C. under N₂ was added trifluoromethylsulfonyl trifluoromethanesulfonate (17.69 g, 62.71 mmol). The reaction was stirred at 0° C. for 2 h then diluted with DCM (200 mL), washed with sat.NaHCO₃ (100 mL×3), water (100 mL×2) and brine (100 mL). The organic layer was dried over Na₂SO₄, filtered and concentrated in vacuo to afford 2-((tert-butyldiphenylsilyl)oxy)ethyl trifluoromethanesulfonate (24.5 g, 99% yield) as a brown oil. This was used without further purification.

Step 3: (35E,37E,39E,40E,46R,47S,48R,49R,51S,53S,55S,56S,57R,58R,67R)-56-[(1R)-2-[(1S,3R,4R)-4-[2-[tert-butyl(diphenyl)silyl]oxyethoxy]-3-methoxy-cyclohexyl]-1-methyl-ethyl]-57,67-dihydroxy-55,58-dimethoxy-46,47,48,49,59,60-hexamethyl-77,78-dioxa-69-azatricyclohexatriaconta-35,37,39(59),40(60)-tetraene-61,62,63,64,65-pentone. To a solution of rapamycin (5 g, 5.47 mmol) and 2-((tert-butyldiphenylsilyl)oxy)ethyl trifluoromethanesulfonate (23.66 g, 54.69 mmol) in toluene (100 mL) was added DIEA (8.48 g, 65.63 mmol). The reaction was stirred at 58° C. for 16 h then poured into cold saturated NaHCO₃ solution (200 mL) and extracted with EtOAc (100 mL×3). The organic layer was washed with water (100 mL×3) and brine (100 mL), dried over anhydrous Na₂SO₄, filtered and concentrated. The residue was purified via silica gel chromatography (PE:EA=3:1) to provide (35E,37E,39E,40E,46R,47S,48R,49R,51S,53S,55S,56S,57R,58R,67R)-56-[(1R)-2-[(1S,3R,4R)-4-[2-[tert-butyl(diphenyl)silyl]oxyethoxy]-3-methoxy-cyclohexyl]-1-methyl-ethyl]-57,67-dihydroxy-55,58-dimethoxy-46,47,48,49,59,60-hexamethyl-77,78-dioxa-69-azatricyclohexatriaconta-35,37,39(59),40(60)-tetraene-61,62,63,64,65-pentone (4.7 g, 72% yield) as a yellow solid. ESI-MS (EI⁺, m/z): 1219.5 [M+Na]⁺.

Step 4: (36E,38E,40E,41E,47R,48S,49R,50R,52S,54S,56S,57S,58R,59R,68R)-57-[(1R)-2-[(1S,3R,4R)-4-[2-[tert-butyl(diphenyl)silyl]oxyethoxy]-3-methoxy-cyclohexyl]-1-methyl-ethyl]-68-hydroxy-56,58,59-trimethoxy-47,48,49,50,60,61-hexamethyl-77,78-dioxa-70-azatricyclohexatriaconta-36,38,40(60),41(61)-tetraene-62,63,64,65,66-pentone. To a solution of (35E,37E,39E,40E,46R,47S,48R,49R,51S,53S,55S,56S,57R,58R,67R)-56-[(1R)-2-[(1S,3R,4R)-4-[2-[tert-butyl(diphenyl)silyl]oxyethoxy]-3-methoxy-cyclohexyl]-1-methyl-ethyl]-57,67-dihydroxy-55,58-dimethoxy-46,47,48,49,59,60-hexamethyl-77,78-dioxa-69-azatricyclohexatriaconta-35,37,39(59),40(60)-tetraene-61,62,63,64,65-pentone (2 g, 1.67 mmol) and 1,8-bis(dimethylamino)naphthalene (3.94 g, 18.39 mmol) in toluene (40 mL) was added methyl trifluoromethanesulfonate (2.19 g, 13.37 mmol) dropwise at room temperature under N₂. The mixture was then heated to 50° C. for 5 hr, filtered and diluted with EA (60 mL), washed with saturated NH₄Cl (aq) (60 mL×3), water (60 mL) and brine (60 mL). The organic layer was dried over anhydrous sodium sulfate, filtered and concentrated. The residue was purified via silica gel chromatography (PE:EA=3:1) to provide (36E,38E,40E,41E,47R,48S,49R,50R,52S,54S,56S,57S,58R,59R,68R)-57-[(1R)-2-[(1S,3R,4R)-4-[2-[tert-butyl(diphenyl)silyl]oxyethoxy]-3-methoxy-cyclohexyl]-1-methyl-ethyl]-68-hydroxy-56,58,59-trimethoxy-47,48,49,50,60,61-hexamethyl-77,78-dioxa-70-azatricyclohexatriaconta-36,38,40(60),41(61)-tetraene-62,63,64,65,66-pentone (700 mg, 35% yield) as a yellow solid. ESI-MS (EI⁺, m/z): 1232.7 [M+Na]

Step 5: (23E,25E,27E,28E,32R,33S,34R,35R,37S,39S,41S,42S,43R,44R,53R)-53-hydroxy-42-[(1R)-2-[(1S,3R,4R)-4-(2-hydroxyethoxy)-3-methoxy-cyclohexyl]-1-methyl-ethyl]-41,43,44-trimethoxy-32,33,34,35,45,46-hexamethyl-62,63-dioxa-54-azatricyclohexatriaconta-23,25,27(45),28(46)-tetraene-47,48,49,50,51-pentone (Intermediate V). To a solution of (36E,38E,40E,41E,47R,48S,49R,50R,52S,54S,56S,57S,58R,59R,68R)-57-[(1R)-2-[(1S,3R,4R)-4-[2-[tert-butyl(diphenyl)silyl]oxyethoxy]-3-methoxy-cyclohexyl]-1-methyl-ethyl]-68-hydroxy-56,58,59-trimethoxy-47,48,49,50,60,61-hexamethyl-77,78-dioxa-70-azatricyclohexatriaconta-36,38,40(60),41(61)-tetraene-62,63,64,65,66-pentone (0.6 g, 0.495 mmol) in THF (10 mL) was added pyridine HF (0.39 g, 4.96 mmol) at 0° C. The mixture was stirred at 30° C. for 3 h then quenched with saturated NaHCO₃ solution (20 mL) and extracted with EA (30 mL) at 0° C. The organic layer was washed with water (20 mL) and brine (20 mL), dried over anhydrous sodium sulfate, filtered and concentrated. The residue was purified via silica gel chromatography (PE:acetone=3:1) to provide Intermediate V (430 mg, 89% yield) as a light yellow solid. ESI-MS (EI⁺, m/z): 994.7 [M+Na]⁺.

### Synthesis of Intermediate VII

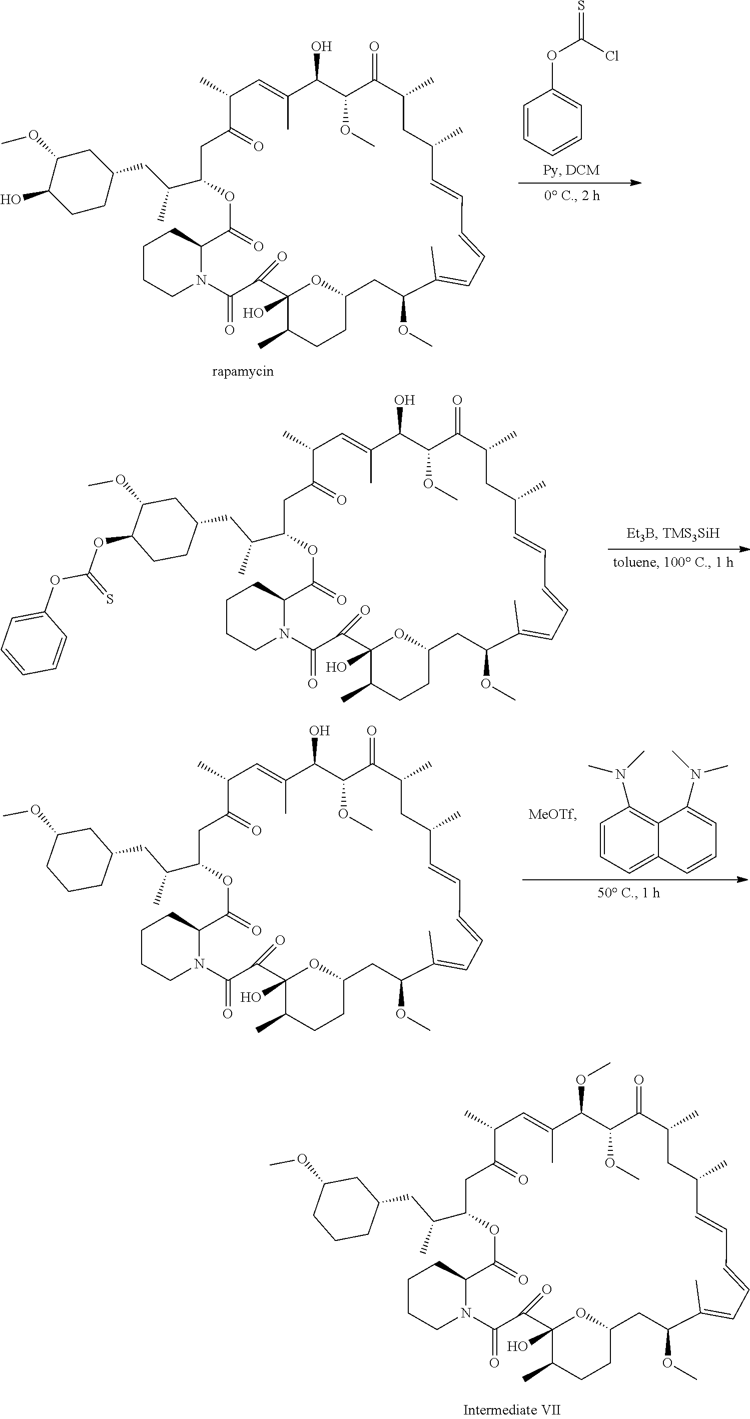

Step 1: (27E,29E,31E,32E,35R,36S,37R,38R,40S,42S,44S,45S,46R,47R,57R)-46,57-dihydroxy-44,47-dimethoxy-45-[(1R)-2-[(1 S,3R,4R)-3-methoxy-4-phenoxycarbothioyloxy-cyclohexyl]-1-methyl-ethyl]-35,36,37,38,48,49-hexamethyl-66,67-dioxa-58-azatricyclohexatriaconta-27,29,31(48),32(49)-tetraene-50,51,52,53,54-pentone. To a solution of (22E,24E,26E,27E,29R,30S,31R,32R,34S,36S,38S,39S,40R,41R,50R)-40,50-dihydroxy-39-[(1R)-2-[(1S,3R,4R)-4-hydroxy-3-methoxy-cyclohexyl]-1-methyl-ethyl]-38,41-dimethoxy-29,30,31,32,42,43-hexamethyl-60,61-dioxa-51-azatricyclohexatriaconta-22,24,26(42),27(43)-tetraene-44,45,46,47,48-pentone (300 mg, 0.328 mmol) in DCM (8 mL) was added pyridine (208 mg, 2.63 mmol) and O-phenyl carbonochloridothioate (283 mg, 1.64 mmol) at 0° C. The resulting solution was stirred at 0° C. for 2 h then diluted with DCM, washed with NH₄Cl, water and brine, dried over Na₂SO₄, filtered and concentrated. The residue was purified via reverse phase chromatography (CH₃CN in water from 0% to 100%) to provide the titled compound (150 mg, 44% yield) as a white solid. ESI-MS (EI⁺, m/z): 1072.3 [M+Na]⁺. ¹H NMR (500 MHz, CDCl₃) δ 7.41 (t, J=7.9 Hz, 2H), 7.29 (d, J=7.4 Hz, 1H), 7.12 (d, J=7.8 Hz, 2H), 6.44-6.09 (m, 3H), 5.96 (d, J=10.4 Hz, 1H), 5.61-5.38 (m, 2H), 5.29 (d, J=5.2 Hz, 1H), 5.22-5.06 (m, 2H), 4.79 (s, 1H), 4.20 (dd, J=16.6, 6.0 Hz, 1H), 3.93-3.52 (m, 4H), 3.51-3.28 (m, 10H), 3.14 (s, 3H), 2.91-2.55 (m, 3H), 2.25 (dd, J=91.2, 12.9 Hz, 4H), 1.97 (d, J=4.8 Hz, 2H), 1.90-1.69 (m, 9H), 1.60 (t, J=22.2 Hz, 11H), 1.54-1.38 (m, 7H), 1.37-1.19 (m, 5H), 1.11 (ddd, J=25.6, 13.0, 7.6 Hz, 10H), 1.01-0.84 (m, 10H).

Step 2: (23E,25E,27E,28E,30R,31S,32R,33R,35S,37S,38S,39S,40R,41R,50R)-40,50-dihydroxy-38,41-dimethoxy-39-[(1R)-2-[(1S,3S)-3-methoxycyclohexyl]-1-methyl-ethyl]-30,31,32,33,42,43-hexamethyl-59,60-dioxa-51-azatricyclohexatriaconta-23,25,27(42),28(43)-tetraene-44,45,46,47,48-pentone. To a solution of (27E,29E,31E,32E,35R,36S,37R,38R,40S,42S,44S,45S,46R,47R,57R)-46,57-dihydroxy-44,47-dimethoxy-45-[(1R)-2-[(1S,3R,4R)-3-methoxy-4-phenoxycarbothioyloxy-cyclohexyl]-1-methyl-ethyl]-35,36,37,38,48,49-hexamethyl-66,67-dioxa-58-azatricyclohexatriaconta-27,29,31(48),32(49)-tetraene-50,51,52,53,54-pentone (1.4 g, 1.33 mmol) in toluene (15 mL) was added triethylborane (157 mg, 1.60 mmol) and bis(trimethylsilyl)silyl-trimethyl-silane (994 mg, 4 mmol, 1M in THF). The resulting solution was stirred at 100° C. for 1 h then concentrated and purified via silica gel chromatography (EtOAc in PE from 0% to 50%) to provide the titled compound (0.6 g, 50% yield) as a light yellow solid. ESI-MS (EI⁺, m/z): 920.0 [M+Na]⁺. ¹H NMR (400 MHz, CDCl₃) δ 6.59-5.85 (m, 4H), 5.68-5.06 (m, 4H), 4.68 (dd, J=48.1, 31.4 Hz, 1H), 4.49-3.99 (m, 2H), 3.99-3.51 (m, 4H), 3.52-3.27 (m, 7H), 3.29-3 (m, 5H), 2.88-2.53 (m, 3H), 2.20 (ddd, J=80.2, 58.5, 14.9 Hz, 6H), 1.80 (dd, J=34.0, 5.5 Hz, 7H), 1.63 (d, J=16.1 Hz, 12H), 1.52-1.19 (m, 10H), 1.21-0.78 (m, 19H), 0.70 (dd, J=16.1, 9.9 Hz, 2H).

Step 3: (24E,26E,28E,29E,31R,32S,33R,34R,36S,38S,39S,40S,41R,42R,51R)-51-hydroxy-39,41,42-trimethoxy-40-[(1R)-2-[(1S,3S)-3-methoxycyclohexyl]-1-methyl-ethyl]-31,32,33,34,43,44-hexamethyl-59,60-dioxa-52-azatricyclohexatriaconta-24,26,28(43),29(44)-tetraene-45,46,47,48,49-pentone (Intermediate VI). To a solution of (23E,25E,27E,28E,30R,31S,32R,33R,35S,37S,38S,39S,40R,41R,50R)-40,50-dihydroxy-38,41-dimethoxy-39-[(1R)-2-[(1S,3S)-3-methoxycyclohexyl]-1-methyl-ethyl]-30,31,32,33,42,43-hexamethyl-59,60-dioxa-51-azatricyclohexatriaconta-23,25,27(42),28(43)-tetraene-44,45,46,47,48-pentone (200 mg, 0.222 mmol) in toluene (8 mL) was added N1,N1,N8,N8-tetramethylnaphthalene-1,8-diamine (668 mg, 3.12 mmol) and methyl trifluoromethanesulfonate (365 mg, 2.23 mmol). The resulting solution was stirred at 50° C. for 1 h then cooled, filtered and concentrated. The residue was purified via silica gel chromatography (45% EtOAc in PE) to provide Intermediate VI (50 mg, 12% yield) as a white solid. ESI-MS (EI⁺, m/z): 934.2 [M+Na]⁺. ¹H NMR (400 MHz, CDCl₃) δ 6.54-5.81 (m, 4H), 5.78-5.02 (m, 5H), 4.52 (dd, J=105.2, 28.6 Hz, 1H), 4.38-3.94 (m, 1H), 3.93-3.53 (m, 4H), 3.54-3.01 (m, 12H), 3.03-2.46 (m, 3H), 2.45-1.88 (m, 6H), 1.90-1.54 (m, 16H), 1.54-1.19 (m, 9H), 1.19-0.76 (m, 16H), 0.70 (d, J=11.0 Hz, 2H).

### COMPOUND SYNTHESIS EXAMPLES

### Example 1: Synthesis of (24E,26E,28E,29E,35R,36S,37R,38R,40S,42S,44S,45R,46R,55R)-55-hydroxy-45,46-dimethoxy-44-[(1R)-2-[(1S,3S)-3-methoxycyclohexyl]-1-methyl-ethyl]-43-[2-(2-methoxyethoxy)ethoxy]-35,36,37,38,47,48-hexamethyl-63,64-dioxa-56-azatricyclohexatriaconta-24,26,28(47),29(48)-tetraene-49,50,51,52,53-pentone (I-1)

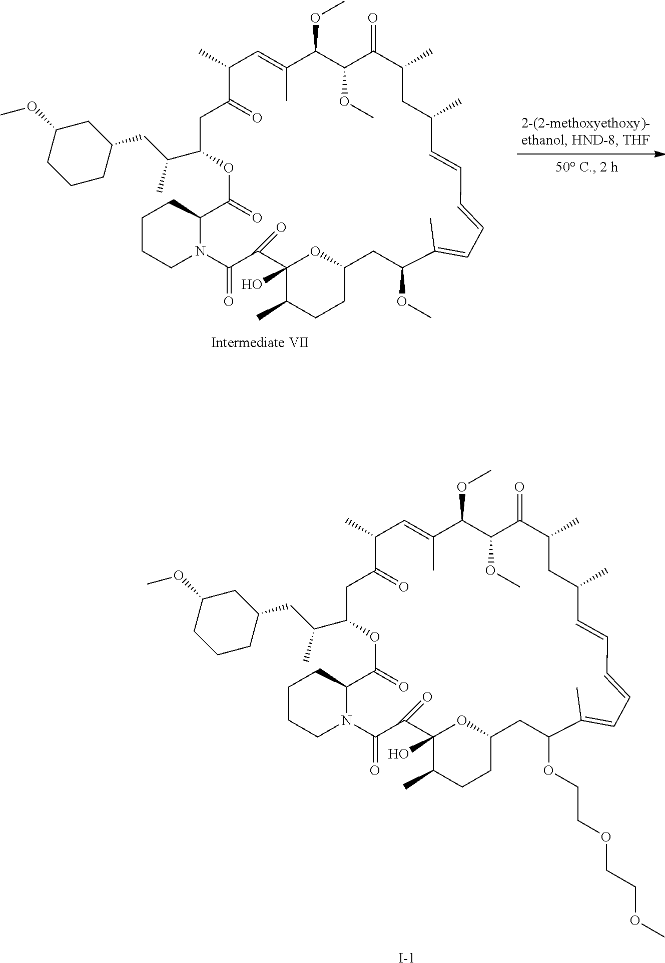

To a solution of Intermediate VII (150 mg, 0.164 mmol) and 2-(2-methoxyethoxy)ethanol (395 mg, 3.29 mmol) in THF (5 mL) was added HND-8 (25 mg) at 50° C. under Ar. The resulting solution was stirred at 50° C. for 2 h, then filtered and concentrated. The residue was purified via reverse phase chromatography (85% CH₃CN in water) to provide the titled compound (I-1: 105 mg, 64% yield) as a white solid. ESI-MS (EI⁺, m/z): 1022.0 [M+Na]⁺. ¹H NMR (500 MHz, CDCl₃) δ 6.51-5.83 (m, 4H), 5.72-5.08 (m, 4H), 4.41 (ddd, J=101.7, 68.7, 23.6 Hz, 2H), 4.01-3.03 (m, 22H), 2.93-2.50 (m, 5H), 2.42-1.70 (m, 17H), 1.52-1.21 (m, 16H), 1.20-0.78 (m, 18H), 0.77-0.65 (m, 1H).

### Example 2: Synthesis of (3S,6R,7E,9R,10R,12R,14S,15E,17E,19E,21S,23S,26R,27R,34aS)-27-hydroxy-9,10-dimethoxy-3-((R)-1-((1S,3S)-3-methoxycyclohexyl)propan-2-yl)-21-(2-((2-methoxyethyl)sulfonyl)ethoxy)-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (I-2)

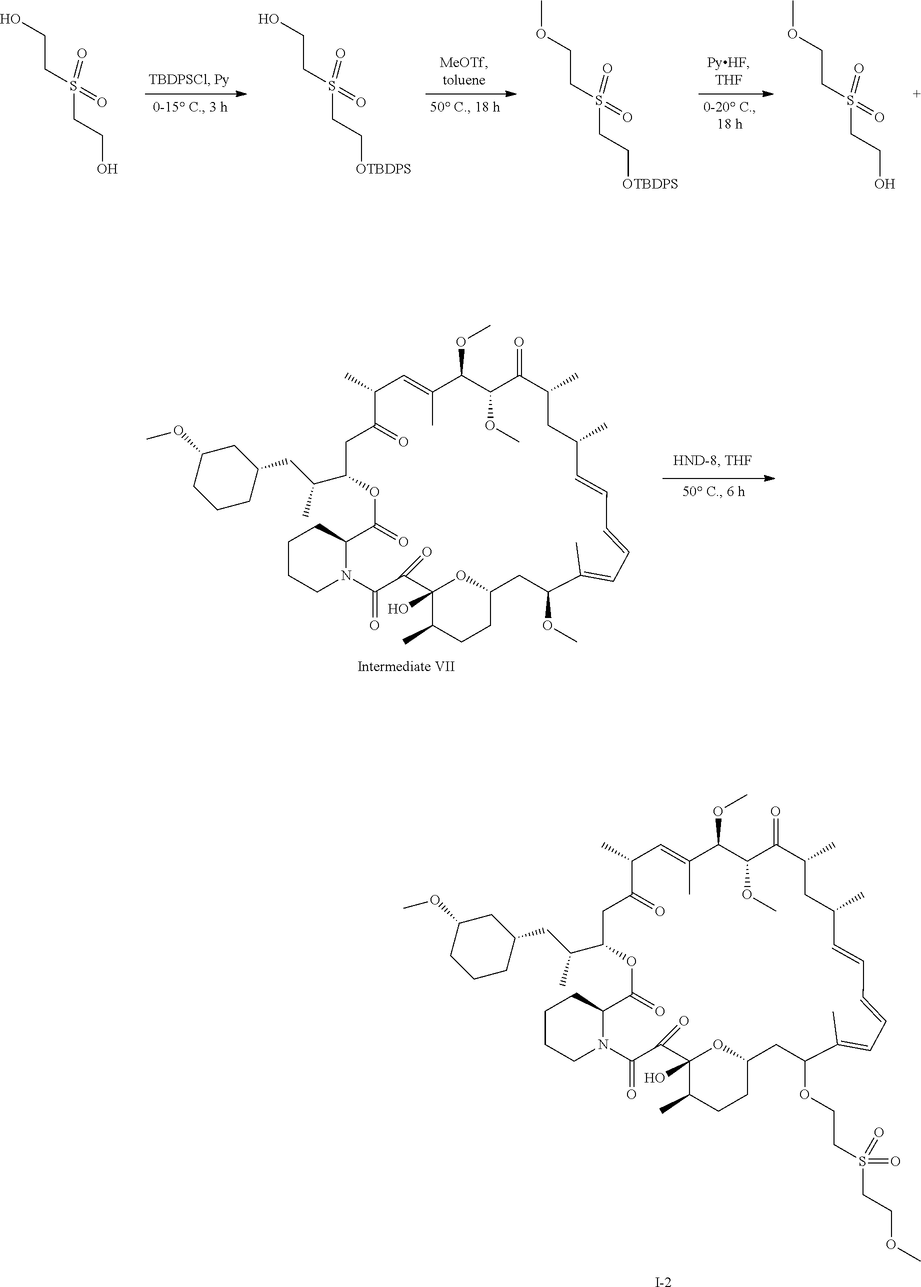

Step 1: 2-[2-[tert-butyl (diphenyl)silyl]oxyethylsulfonyl]ethanol. To a solution of 2-(2-hydroxyethylsulfonyl) ethanol (5.01 g, 32.47 mmol) in pyridine (20 mL) was added tert-butyl-chloro-diphenyl-silane (2.22 g, 8.08 mmol) at 0° C. The reaction was stirred at 15° C. for 3 h then diluted with water (200 mL) and extracted with EtOAc (100 mL×3). The combined organic layers were concentrated and purified via silica gel chromatography (EtOAc:PE=1:2) to provide 2-[2-[tert-butyl(diphenyl)silyl]oxyethylsulfonyl]ethanol (2.25 g, 71% yield) as a white solid. ¹HNMR (400 MHz, CDCl₃): δ 7.65-7.67 (m, 4H), 7.42-7.47 (m, 6H), 4.09-4.14 (m, 4H), 3.44-3.46 (m, 2H), 3.25-3.27 (m, 2H), 2.57-2.60 (m, 1H), 1.06 (s, 9H).

Step 2: tert-butyl-[2-(2-methoxyethylsulfonyl) ethoxy]-diphenyl-silane. To a solution of 2-[2-[tert-butyl(diphenyl)silyl]oxyethylsulfonyl]ethanol (8.6 g, 21.91 mmol) and N1,N1,N8,N8-tetramethylnaphthalene-1,8-diamine (14.08 g, 65.72 mmol) in toluene (20 mL) was added methyl trifluoromethanesulfonate (10.78 g, 65.72 mmol,) at 0° C. The mixture was stirred at 50° C. for 18 h then concentrated, treated with water (200 mL) and extracted with EtOAc (150 mL×2). The combined organic layers were concentrated and purified via silica gel column chromatography (PE:EtOAc=3:1) to provide tert-butyl-[2-(2-methoxyethylsulfonyl) ethoxy]-diphenyl-silane (7.9 g, 89% yield) as a white solid. ESI-MS (EI⁺, m/z): 429.0 [M+Na]⁺. ¹HNMR (400 MHz, CDCl₃): δ 7.67-7.69 (m, 4H), 7.39-7.45 (m, 6H), 4.07-4.10 (m, 2H), 3.82-3.84 (m, 2H), 3.40-3.43 (m, 2H), 3.37 (s, 3H), 3.29-3.31 (m, 2H), 1.06 (s, 9H).

Step 3: 2-(2-methoxyethylsulfonyl) ethanol. To a solution of tert-butyl-[2-(2-methoxyethylsulfonyl) ethoxy]-diphenyl-silane (8.6 g, 21.15 mmol) in THF (10 mL) was added Py.HF (31.44 g, 317.26 mmol). The mixture was stirred at 20° C. for 18 h then concentrated and treated with EtOAc (100 mL). NaHCO₃ (aq. 150 mL) was added, the mixture was stirred at rt for 1 h then filtered and washed with EtOAc (20 mL). The combined organic layers were concentrated and purified via reverse-phase chromatography (water) to provide 2-(2-methoxyethylsulfonyl) ethanol (3.55 g, 99% yield) as yellow oil. ESI-MS (EI⁺, m/z): 169.0 [M+H]⁺. ¹H NMR (400 MHz, CDCl₃): δ 4.09-4.13 (m, 2H), 3.83-3.86 (m, 2H), 3.40 (s, 3H), 3.31-3.37 (m, 4H), 2.68-2.71 (m, 1H).

Step 4: (24E,26E,28E,29E,35R,36S,37R,38R,40S,42S,44S,45R,46R,55R)-55-hydroxy-45,46-dimethoxy-44-[(1R)-2-[(1S,3S)-3-methoxycyclohexyl]-1-methyl-ethyl]-43-[2-(2-methoxyethylsulfonyl)ethoxy]-35,36,37,38,47,48-hexamethyl-65,66-dioxa-56-azatricyclohexatriaconta-24,26,28(47),29(48)-tetraene-49,50,51,52,53-pentone (I-2). To a solution of Intermediate VII (200 mg, 0.22 mmol) and 2-(2-methoxyethylsulfonyl)ethanol (369 mg, 2.19 mmol) in THF (3 mL) was added HND-8 (80 mg) at 50° C. The mixture was stirred at 50° C. for 6 h then quenched with saturated NaHCO₃ (aq.) (20 mL) and extracted with EtOAc (30 mL). The organic layer was washed with water (20 mL) and brine (20 mL), dried over anhydrous sodium sulfate, filtered and concentrated. The residue was purified via reverse phase chromatography (0-100% CH₃CN in water) to provide the titled compound (1-2, 25 mg, 11% yield). ESI-MS (EI⁺, m/z): 1069.7 [M+Na]⁺. ¹H NMR (400 MHz, CDCl₃): δ 6.05-6.43 (m, 4H), 5.10-5.59 (m, 4H), 4.41-4.44 (m, 1H), 3.53-3.90 (m, 7H), 3.24-3.46 (m, 15H), 3.03-3.17 (m, 4H), 2.89-2.95 (m, 1H), 2.70-2.78 (m, 1H), 2.51-2.69 (m, 2H), 2.17-2.34 (m, 4H), 1.94-2.15 (m, 4H), 1.54-1.89 (m, 25H), 1.22-1.53 (m, 12H), 1.01-1.20 (m, 12H), 0.84-0.96 (m, 8H), 0.69-0.82 (m, 1H).

### Example 3: Synthesis of (22E,24E,26E,27E,33R,34S,35R,36R,38S,40S,43S,44R,45R,55R)-43-[(1R)-2-[(1S,3R,4R)-4-(difluoromethoxy)-3-methoxy-cyclohexyl]-1-methyl-ethyl]-44,55-dihydroxy-45-methoxy-42-[2-(2-methoxyethoxy)ethoxy]-33,34,35,36,46,47-hexamethyl-64,65-dioxa-56-azatricyclohexatriaconta-22,24,26(46),27(47)-tetraene-48,49,50,51,52-pentone (I-3), (22E,24E,26E,27E,33R,34S,35R,36R,38S,40S,42S,43S,44R,45R,55R)-43-[(1R)-2-[(1S,3R,4R)-4-(difluoromethoxy)-3-methoxy-cyclohexyl]-1-methyl-ethyl]-44,55-dihydroxy-45-methoxy-42-[2-(2-methoxyethoxy)ethoxy]-33,34,35,36,46,47-hexamethyl-64,65-dioxa-56-azatricyclohexatriaconta-22,24,26(46),27(47)-tetraene-48,49,50,51,52-pentone (I-4) and (22E,24E,26E,27E,33R,34S,35R,36R,38S,40S,42R,43S,44R,45R,55R)-43-[(1R)-2-[(1S,3R,4R)-4-(difluoromethoxy)-3-methoxy-cyclohexyl]-1-methyl-ethyl]-44,55-dihydroxy-45-methoxy-42-[2-(2-methoxyethoxy)ethoxy]-33,34,35,36,46,47-hexamethyl-64,65-dioxa-56-azatricyclohexatriaconta-22,24,26(46),27(47)-tetraene-48,49,50,51,52-pentone (I-5)

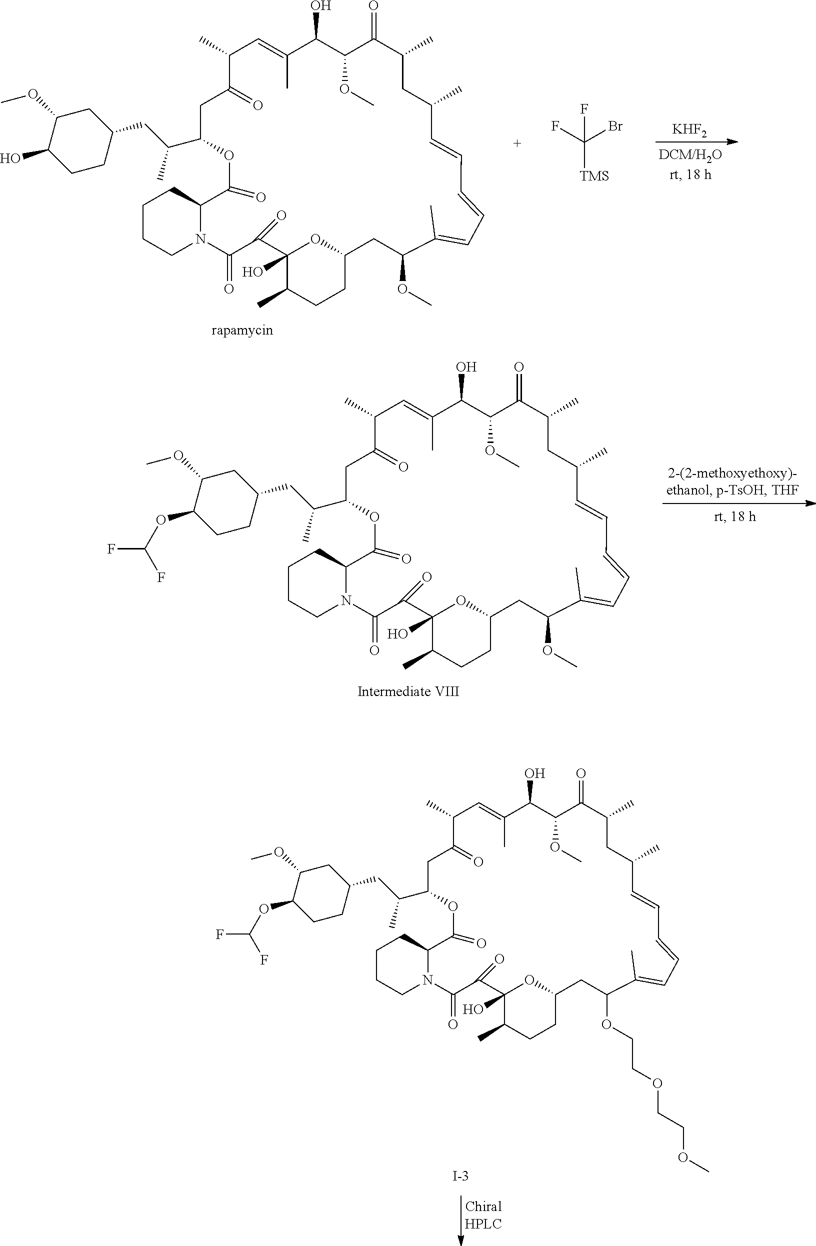

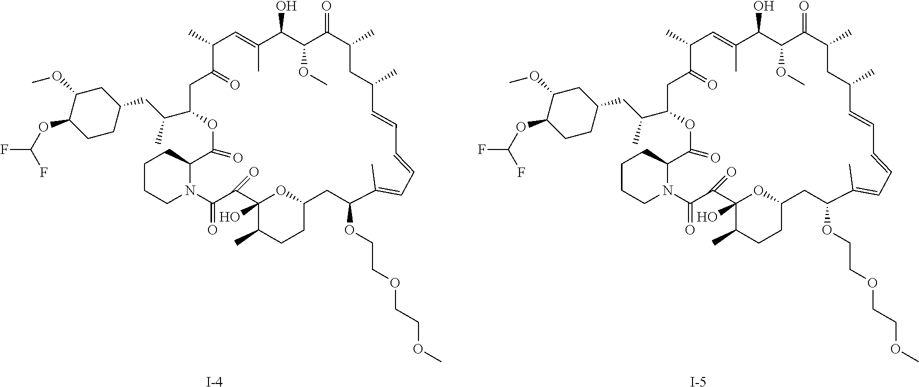

Step 1: (22E,24E,26E,27E,29R,30S,31R,32R,34S,36S,38S,39S,40R,41R,51R)-39-[(1R)-2-[(1S,3R,4R)-4-(difluoromethoxy)-3-methoxy-cyclohexyl]-1-methyl-ethyl]-40,51-dihydroxy-38,41-dimethoxy-29,30,31,32,42,43-hexamethyl-60,61-dioxa-52-azatricyclohexatriaconta-22,24,26(42),27(43)-tetraene-44,45,46,47,48-pentone (Intermediate VIII). To a solution of (22E,24E,26E,27E,29R,30S,31R,32R,34S,36S,38S,39S,40R,41R,50R)-40,50-dihydroxy-39-[(1R)-2-[(1S,3R,4R)-4-hydroxy-3-methoxy-cyclohexyl]-1-methyl-ethyl]-38,41-dimethoxy-29,30,31,32,42,43-hexamethyl-60,61-dioxa-51-azatricyclohexatriaconta-22,24,26(42),27(43)-tetraene-44,45,46,47,48-pentone (1.0 g, 1.09 mmol) in DCM (15 mL) at rt was added potassium fluoride hydrofluoride (1.28 g, 16.41 mmol) in water (15 mL) and bromodifluoro(trimethylsilyl)methane (2.22 g, 10.94 mmol). The reaction was stirred at 25° C. for 18 h then diluted with DCM, washed with saturated aqueous NH₄Cl solution, water, brine, dried over Na₂SO₄, filtered and concentrated. The residue was purified via silica gel chromatography (EtOAc:PE=1:1.2) to obtain the titled compound (110 mg, 10% yield) as a white solid. ESI-MS (EI⁺, m/z): 985.8 [M+Na]⁺. ¹H NMR (400 MHz, CDCl₃) δ 6.72-5.83 (m, 5H), 5.62 (ddd, J=22.9, 14.6, 7.9 Hz, 1H), 5.49-5.01 (m, 3H), 4.67 (s, 1H), 3.98-3.54 (m, 6H), 3.52-3.05 (m, 15H), 2.88-2.52 (m, 3H), 2.41-1.68 (m, 16H), 1.56-1.19 (m, 10H), 1.17-0.86 (m, 17H), 0.76 (dd, J=24.3, 12.0 Hz, 2H).

Step 2: (22E,24E,26E,27E,33R,34S,35R,36R,38S,40S,43S,44R,45R,55R)-43-[(1R)-2-[(1S,3R,4R)-4-(difluoromethoxy)-3-methoxy-cyclohexyl]-1-methyl-ethyl]-44,55-dihydroxy-45-methoxy-42-[2-(2-methoxyethoxy)ethoxy]-33,34,35,36,46,47-hexamethyl-64,65-dioxa-56-azatricyclohexatriaconta-22,24,26(46),27(47)-tetraene-48,49,50,51,52-pentone (CP-NAV-067-1410). To a solution of (22E,24E,26E,27E,29R,30S,31R,32R,34S,36S,38S,39S,40R,41R,51R)-39-[(1R)-2-[(1S,3R,4R)-4-(difluoromethoxy)-3-methoxy-cyclohexyl]-1-methyl-ethyl]-40,51-dihydroxy-38,41-dimethoxy-29,30,31,32,42,43-hexamethyl-60,61-dioxa-52-azatricyclohexatriaconta-22,24,26(42),27(43)-tetraene-44,45,46,47,48-pentone (200 mg, 0.21 mmol) and 2-(2-methoxyethoxy)ethanol (498 mg, 4.15 mmol) in THF (5 mL) at 0° C. under N₂ was added 4-methylbenzenesulfonic acid hydrate (197 mg, 1.04 mmol). The reaction was stirred at this temperature for 2 h then diluted with ice cold aqueous NaHCO₃ solution, extracted with EtOAc, washed with brine, dried over Na₂SO₄, filtered and concentrated. The residue was purified via reverse phase chromatography (76% CH₃CN in water) to provide the titled compound (I-3: 40 mg, 18% yield) as a white solid.

Step 3: (22E,24E,26E,27E,33R,34S,35R,36R,38S,40S,42S,43S,44R,45R,55R)-43-[(1R)-2-[(1S,3R,4R)-4-(difluoromethoxy)-3-methoxy-cyclohexyl]-1-methyl-ethyl]-44,55-dihydroxy-45-methoxy-42-[2-(2-methoxyethoxy)ethoxy]-33,34,35,36,46,47-hexamethyl-64,65-dioxa-56-azatricyclohexatriaconta-22,24,26(46),27(47)-tetraene-48,49,50,51,52-pentone (CP-NAV-067-1429-P1) and (22E,24E,26E,27E,33R,34S,35R,36R,38S,40S,42R,43S,44R,45R,55R)-43-[(1R)-2-[(1S,3R,4R)-4-(difluoromethoxy)-3-methoxy-cyclohexyl]-1-methyl-ethyl]-44,55-dihydroxy-45-methoxy-42-[2-(2-methoxyethoxy)ethoxy]-33,34,35,36,46,47-hexamethyl-64,65-dioxa-56-azatricyclohexatriaconta-22,24,26(46),27(47)-tetraene-48,49,50,51,52-pentone (CP-NAV-067-1429-P2). 100 mg of the mixture was separated via chiral HPLC and then purified via silica gel chromatography (PE:DCM:EtOAc:MeOH=3:3:1:0.3) to provide the titled compound (I-4: 28 mg, 28% yield) and (I-5: 15 mg, 15% yield) as white solids.

Chiral separation method:

### Column: CHIRALPAK IC

Column size: 5.0 cm I.D.×25 cm L, 10 μm
Sample solution: 1 mg/mL in Mobile phase

### Injection: 5 mL

Mobile phase: Hexane/EtOH=70/30(V/V)
Flow rate: 30 mL/min
Wave length: UV 254 nm

### Temperature: 38° C.

I-4: ESI-MS (EI⁺, m/z): 1073.7 [M+Na]⁺. ¹H NMR (500 MHz, CDCl₃) δ 6.60-6.05 (m, 4H), 5.91 (dd, J=41.4, 11.1 Hz, 1H), 5.58-5.07 (m, 4H), 4.74 (s, 1H), 4.19 (dd, J=14.0, 6.0 Hz, 1H), 3.95-3.26 (m, 24H), 3.12 (dd, J=16.8, 7.9 Hz, 1H), 2.92-2.51 (m, 3H), 2.40-1.86 (m, 8H), 1.84-1.64 (m, 11H), 1.54-1.16 (m, 10H), 1.16-0.83 (m, 18H), 0.78-0.65 (m, 1H).
I-5: ESI-MS (EI⁺, m/z): 1073.7 [M+Na]⁺. ¹H NMR (500 MHz, CDCl₃) δ 6.64-5.88 (m, 5H), 5.75-5.08 (m, 5H), 4.28 (s, 1H), 4.03-3.02 (m, 26H), 2.98-1.90 (m, 9H), 1.86-1.63 (m, 16H), 1.50-1.17 (m, 6H), 1.16-0.81 (m, 18H), 0.78-0.61 (m, 1H).

### Example 4: (24E,26E,28E,29E,32R,33S,34R,35R,37S,39S,42S,44R,45R,55R)-42-[(1R)-2-[(1S,3R,4R)-4-(difluoromethoxy)-3-methoxy-cyclohexyl]-1-methyl-ethyl]-41-(1,4-dioxan-2-ylmethoxy)-44,55-dihydroxy-45-methoxy-32,33,34,35,46,47-hexamethyl-66,67-dioxa-56-azatricyclohexatriaconta-24,26,28(46),29(47)-tetraene-48,49,50,51,52-pentone (I-6), (24E,26E,28E,29E,32R,33S,34R,35R,37S,39S,41S,42S,44R,45R,55R)-42-[(1R)-2-[(1S,3R,4R)-4-(difluoromethoxy)-3-methoxy-cyclohexyl]-1-methyl-ethyl]-41-(1,4-dioxan-2-ylmethoxy)-44,55-dihydroxy-45-methoxy-32,33,34,35,46,47-hexamethyl-66,67-dioxa-56-azatricyclohexatriaconta-24,26,28(46),29(47)-tetraene-48,49,50,51,52-pentone (I-9) and (24E,26E,28E,29E,32R,33S,34R,35R,37S,39S,41R,42S,44R,45R,55R)-42-[(1R)-2-[(1S,3R,4R)-4-(difluoromethoxy)-3-methoxy-cyclohexyl]-1-methyl-ethyl]-41-(1,4-dioxan-2-ylmethoxy)-44,55-dihydroxy-45-methoxy-32,33,34,35,46,47-hexamethyl-66,67-dioxa-56-azatricyclohexatriaconta-24,26,28(46),29(47)-tetraene-48,49,50,51,52-pentone (I-10)

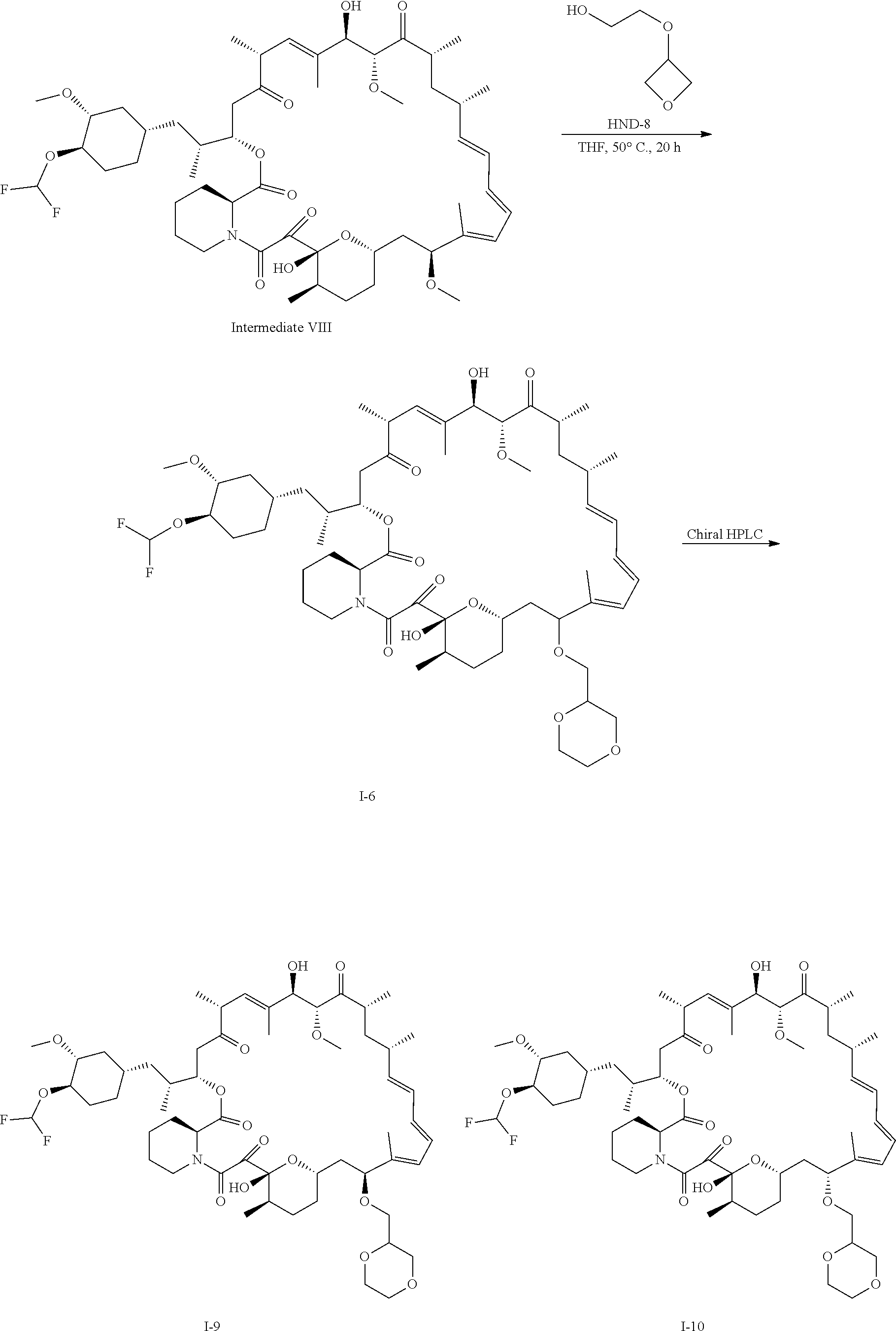

Step 1: (24E,26E,28E,29E,32R,33S,34R,35R,37S,39S,42S,44R,45R,55R)-42-[(1R)-2-[(1S,3R,4R)-4-(difluoromethoxy)-3-methoxy-cyclohexyl]-1-methyl-ethyl]-41-(1,4-dioxan-2-ylmethoxy)-44,55-dihydroxy-45-methoxy-32,33,34,35,46,47-hexamethyl-66,67-dioxa-56-azatricyclohexatriaconta-24,26,28(46),29(47)-tetraene-48,49,50,51,52-pentone (I-6). To a solution of (22E,24E,26E,27E,29R,30S,31R,32R,34S,36S,38S,39S,40R,41R,51R)-39-[(1R)-2-[(1S,3R,4R)-4-(difluoromethoxy)-3-methoxy-cyclohexyl]-1-methyl-ethyl]-40,51-dihydroxy-38,41-dimethoxy-29,30,31,32,42,43-hexamethyl-60,61-dioxa-52-azatricyclohexatriaconta-22,24,26(42),27(43)-tetraene-44,45,46,47,48-pentone (0.1 g, 0.1 mmol, from Example 3) and 2-(oxetan-3-yloxy)ethanol (245 mg, 2.07 mmol) in THF (10 mL) at 50° C. under N₂ was added HND-8 (50 mg). The reaction mixture was stirred for 20 h at 50° C., cooled, filtered and the filtrate was poured into saturated aqueous NaHCO₃ (2 mL) at 0° C. and extracted with EtOAc (20 mL). The organic layer was washed with water (20 mL) and brine (20 mL), dried over anhydrous sodium sulfate, filtered and concentrated. The residue was purified via silica gel chromatography (EtOAc:PE=4:1) and reverse phase chromatography eluting with 60% CH₃CN in water to provide the titled compound (30 mg, 28% yield) as a white solid. ESI-MS (EI⁺, m/z): 1072.5 [M+Na]⁺. 1H NMR (400 MHz, CDCl₃) δ 6.58-5.92 (m, 5H), 5.53-4.75 (m, 5H), 4.27-4.09 (m, 2H), 3.84-3.67 (m, 9H), 3.63-3.54 (m, 2H), 3.45-3.28 (m, 10H), 3.25-3.07 (m, 3H), 2.84-2.55 (m, 3H), 2.35-2.20 (m, 2H), 2.13-1.86 (m, 6H), 1.46-1.77 (m, 37H), 1.43-1.17 (m, 14H), 1.11-0.82 (m, 22H), 0.79-0.69 (m, 1H).

Step 2: (24E,26E,28E,29E,32R,33S,34R,35R,37S,39S,41S,42S,44R,45R,55R)-42-[(1R)-2-[(1S,3R,4R)-4-(difluoromethoxy)-3-methoxy-cyclohexyl]-1-methyl-ethyl]-41-(1,4-dioxan-2-ylmethoxy)-44,55-dihydroxy-45-methoxy-32,33,34,35,46,47-hexamethyl-66,67-dioxa-56-azatricyclohexatriaconta-24,26,28(46),29(47)-tetraene-48,49,50,51,52-pentone (I-9) and (24E,26E,28E,29E,32R,33S,34R,35R,37S,39S,41R,42S,44R,45R,55R)-42-[(1R)-2-[(1S,3R,4R)-4-(difluoromethoxy)-3-methoxy-cyclohexyl]-1-methyl-ethyl]-41-(1,4-dioxan-2-ylmethoxy)-44,55-dihydroxy-45-methoxy-32,33,34,35,46,47-hexamethyl-66,67-dioxa-56-azatricyclohexatriaconta-24,26,28(46),29(47)-tetraene-48,49,50,51,52-pentone (I-10). 115 mg of the mixture was separated via chiral HPLC and then purified via silica gel chromatography (PE:DCM:EtOAc:MeOH=3:3:1:0.3) to provide the titled compounds I-9 (35 mg, 30% yield) and I-10 (12 mg, 10% yield) as white solids.

Chiral separation method:

Column: CHIRALPAK IC

Column size: 5.0 cm I.D.×25 cm L, 10 μm

Sample solution: 0.2 mg/mL in Mobile phase

Injection: 5 mL

Mobile phase: Hexane/EtOH=60/40(V/V)

Flow rate: 30 mL/min

Wave length: UV 266 nm

Temperature: 35° C.

I-9: ESI-MS (EI⁺, m/z): 1072.5 [M+Na]⁺. ¹H NMR (400 MHz, CDCl₃) δ 6.59-6.10 (m, 4H), 5.91 (dd, J=28.2, 10.4 Hz, 1H), 5.58-5.06 (m, 4H), 4.75 (dd, J=16.6, 9.8 Hz, 1H), 4.69-4.53 (m, 1H), 4.17 (d, J=5.7 Hz, 1H), 3.91-3.54 (m, 12H), 3.48-3.01 (m, 13H), 2.91-2.53 (m, 3H), 2.38-1.81 (m, 7H), 1.83-1.64 (m, 9H), 1.52-1.19 (m, 10H), 1.16-0.81 (m, 18H), 0.74 (dd, J=24.3, 12.0 Hz, 1H).
I-10: ESI-MS (EI⁺, m/z): 1072.5 [M+Na]⁺. ¹H NMR (400 MHz, CDCl₃) δ 6.67-5.84 (m, 5H), 5.70-5.07 (m, 4H), 4.37-4.07 (m, 3H), 3.98 (t, J=4.3 Hz, 1H), 3.87-3.57 (m, 8H), 3.56-3.05 (m, 13H), 2.93-1.97 (m, 10H), 1.94-1.64 (m, 15H), 1.54-1.20 (m, 7H), 1.18-0.83 (m, 18H), 0.77-0.61 (m, 1H).

### Example 5: Synthesis of (24E,26E,28E,29E,35R,36S,37R,38R,40S,42S,45S,47R,48R,57R)-47,57-dihydroxy-48-methoxy-44-[2-(2-methoxyethoxy)ethoxy]-45-[(1R)-2-[(1S,3R)-3-methoxy-4-(oxetan-3-yloxy)cyclohexyl]-1-methyl-ethyl]-35,36,37,38,49,50-hexamethyl-67,68-dioxa-58-azatricyclohexatriaconta-24,26,28(49),29(50)-tetraene-51,52,53,54,55-pentone (I-7)

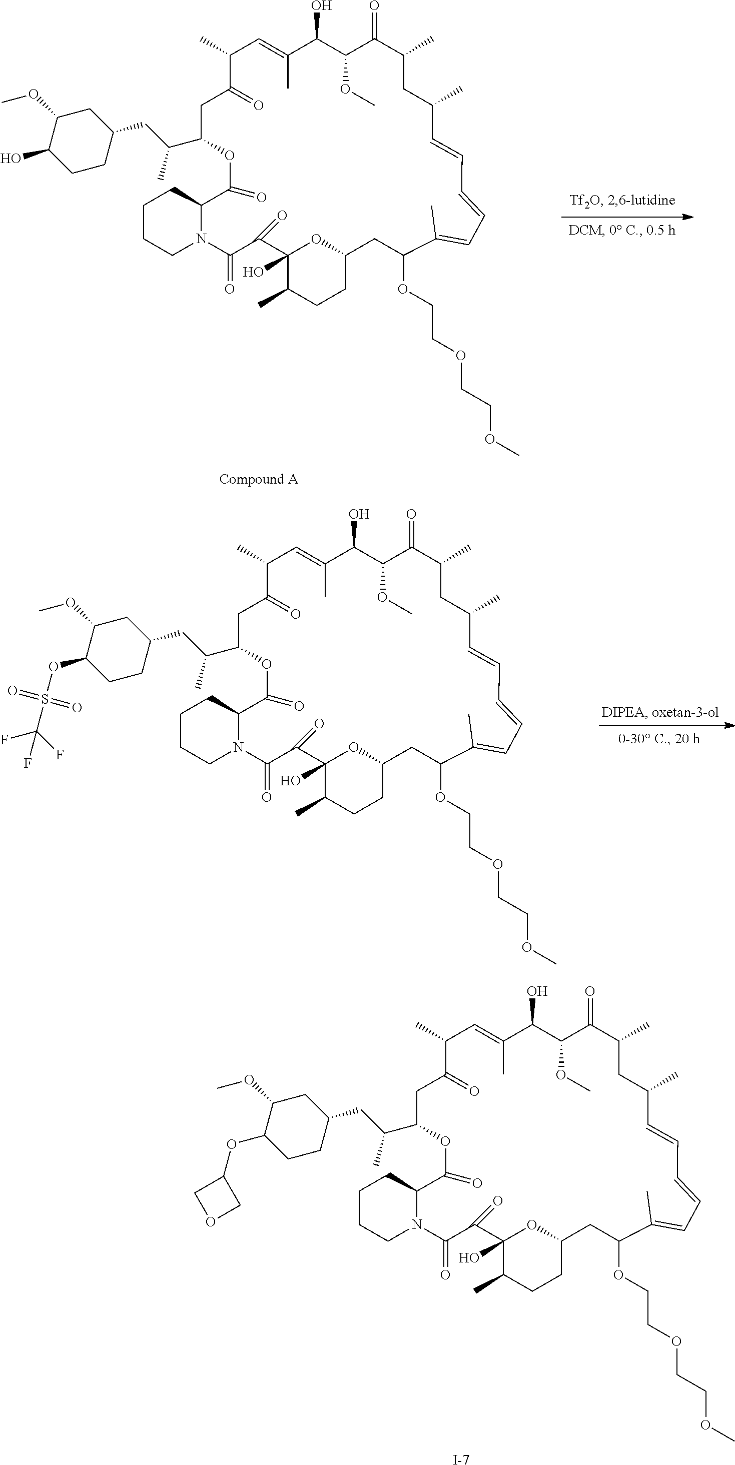

Step 1: [(37S,39R,41R)-4-[(2R)-(22E,24E,26E,27E,33R,34S,35R,36R,38S,40S,43S,44R,45R,54R)-44,54-dihydroxy-45-methoxy-42-[(2-(2methoxyethoxy)ethoxy]-33,34,35,36,46,47-hexamethyl-48,49,50,51,52-pentaoxo-66,67-dioxa-56-azatricyclohexatriaconta-22,24,26(46),27(47)-tetra-43-yl]propyl]-41-methoxy-39-cyclohexyl]trifluoromethanesulfonate. To a solution of (22E,24E,26E,27E,33R,34S,35R,36R,38S,40S,43S,44R,45R,54R)-44,54-dihydroxy-43-[(1R)-2-[(1S,3R,4R)-4-hydroxy-3-methoxy-cyclohexyl]-1-methyl-ethyl]-45-methoxy-42-[2-(2-methoxyethoxy)ethoxy]-33,34,35,36,46,47-hexamethyl-64,65-dioxa-55-azatricyclohexatriaconta-22,24,26(46),27(47)-tetraene-48,49,50,51,52-pentone (Compound A was prepared according to U.S. Pat. No. 10,980,784, 1 g, 1 mmol) and 2,6-dimethyl pyridine (1.07 g, 10 mmol) in DCM (10 mL) was added trifluoromethanesulfonic anhydride (1.41 g, 4.99 mmol) (dissolved in 1 mL DCM) dropwise at 0° C. under N₂. The reaction was stirred for 0.5 h at 0° C. The reaction mixture was used directly in the next step without further purification.

Step 2: (24E,26E,28E,29E,35R,36S,37R,38R,40S,42S,45S,47R,48R,57R)-47,57-dihydroxy-48-methoxy-44-[2-(2-methoxyethoxy)ethoxy]-45-[(1R)-2-[(1S,3R)-3-methoxy-4-(oxetan-3-yloxy)cyclohexyl]-1-methyl-ethyl]-35,36,37,38,49,50-hexamethyl-67,68-dioxa-58-azatricyclohexatriaconta-24,26,28(49),29(50)-tetraene-51,52,53,54,55-pentone (I-7). The reaction solution from step 1 was cooled to 0° C. under N₂ and DIPEA (1.29 g, 9.96 mmol) and oxetan-3-ol (0.74 g, 9.96 mmol) was added. The reaction was warmed to rt and stirred for 20 h then concentrated and purified via silica gel chromatography (80% EtOAc in PE) and reverse phase chromatography (eluting with 60% CH₃CN in water) to provide the titled compound (0.055 g, 5% yield) as a white solid. ESI-MS (EI⁺, m/z): 1079.9 [M+Na]⁺. 1H NMR (400 MHz, CDCl₃) δ 6.39-5.94 (m, 4H), 5.54-5.12 (m, 4H),4.79-4.49 (m, 4H), 4.27-3.98 (m, 2H), 3.91-3.74 (m, 3H), 3.63-3.52 (m, 9H), 3.50-3.12 (m, 13H), 2.81-2.49 (m, 3H), 2.26-1.97 (m, 4H), 1.91-1.49 (m, 29H), 1.53-1.12 (m, 12H), 1.14-0.84 (m, 15H).

### Example 6: Synthesis of (25E,27E,29E,30E,33R,34S,35R,36R,38S,40S,43S,45R,46R,56R)-43-[(1R)-2-[(1S,3R,4R)-4-(difluoromethoxy)-3-methoxy-cyclohexyl]-1-methyl-ethyl]-42-(1,4-dioxan-2-ylmethoxy)-56-hydroxy-45,46-dimethoxy-33,34,35,36,47,48-hexamethyl-66,67-dioxa-57-azatricyclohexatriaconta-25,27,29(47),30(48)-tetraene-49,50,51,52,53-pentone (I-8)

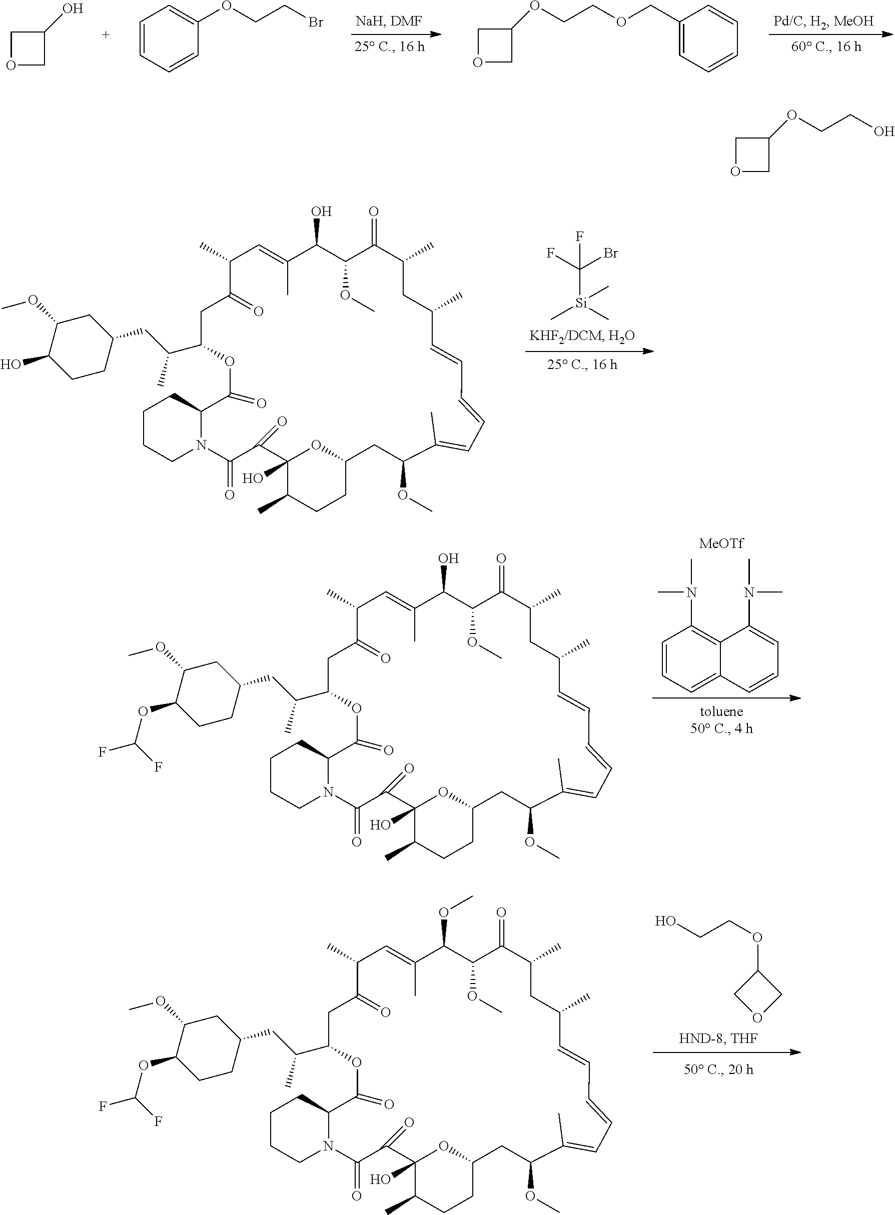

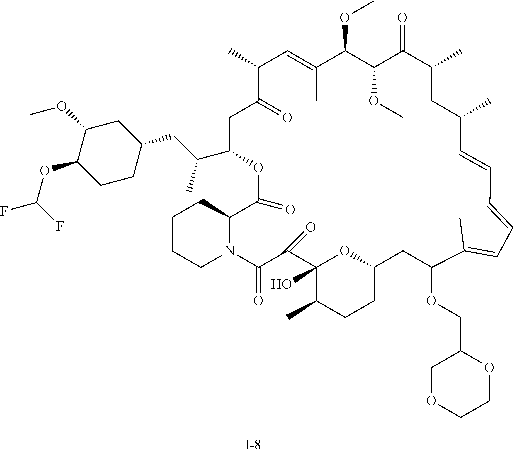

Step 1: 3-(2-benzyloxyethoxy) oxetane. To a solution of oxetan-3-ol (8 g, 108 mmol) and 2-bromoethoxymethylbenzene (34.84 g, 162 mmol) in DMF (20 mL) was added sodium hydride (5.18 g, 216 mmol) batchwise. The resulting solution was stirred for 2 h at 0° C. and for 16 h at room temperature. The reaction was then quenched with 50 mL of NH₄Cl (sat. aq.) then extracted with EtOAc (50 mL×2) and the organic layers were combined and concentrated. The residue was purified via silica gel chromatography eluting with PE:EtOAc (8:1) to provide 3-(2-benzyloxyethoxy)oxetane (12.4 g, 55% yield). ¹H NMR (400 MHz, CDCl₃) δ 7.32 (s, 4H), 7.31-7.26 (m, 1H), 4.72 (dd, J=6.3, 5.7 Hz, 2H), 4.64-4.60 (m, 2H), 4.58 (dd, J=8.6, 3.0 Hz, 1H), 4.54 (s, 2H), 3.57 (dt, J=5.6, 2.7 Hz, 5H).

Step 2: 2-(oxetan-3-yloxy) ethanol. To a solution of 3-(2-benzyloxyethoxy) oxetane (8 g, 38.41 mmol) in MeOH (20 mL) was added Pd/C (4.09 g, 38.41 mmol, 10%) batchwise. The resulting solution was stirred at 60° C. for 16 h then filtered and concentrated. The residue was purified via silica gel chromatography (PE:EtOAc=1:5) to provide 2-(oxetan-3-yloxy) ethanol (2.96 g, 65% yield). ¹H NMR (400 MHz, CDCl₃) δ 4.79 (dd, J=8.3, 4.3 Hz, 2H), 4.62 (dt, J=10.1, 4.9 Hz, 3H), 3.75 (d, J=3.9 Hz, 2H), 3.54-3.45 (m, 2H), 2.44 (d, J=5.9 Hz, 1H).

Step 3: (22E,24E,26E,27E,29R,30S,31R,32R,34S,36S,38S,39S,40R,41R,51R)-39-[(1R)-2-[(1S,3R,4R)-4-(difluoromethoxy)-3-methoxy-cyclohexyl]-1-methyl-ethyl]-40,51-dihydroxy-38,41-dimethoxy-29,30,31,32,42,43-hexamethyl-60,61-dioxa-52-azatricyclohexatriaconta-22,24,26(42),27(43)-tetraene-44,45,46,47,48-pentone: To a solution of rapamycin (1 g, 1.09 mmol) in DCM (15 mL) at rt was added bromodifluoro(trimethylsilyl)methane (2.22 g, 10.94 mmol) dissolved in 30 mL water. The reaction was stirred for 16 h at rt then poured into ice cold saturated aqueous NaHCO₃ (10 mL). The organic layer was washed with water (10 mL×3) and brine (10 mL×3), dried over anhydrous sodium sulfate, filtered and concentrate. The residue was purified via silica gel chromatography (EtOAc:PE=1:1) to provide the titled compound (200 mg, 19% yield) as a white solid. ESI-MS (EI⁺, m/z): 986.5 [M+Na]⁺, T=2.428 min.

Step 4: (23E,25E,27E,28E,30R,31S,32R,33R,35S,37S,39S,40S,41R,42R,52R)-40-[(1R)-2-[(1S,3R,4R)-4-(difluoromethoxy)-3-methoxy-cyclohexyl]-1-methyl-ethyl]-52-hydroxy-39,41,42-trimethoxy-30,31,32,33,43,44-hexamethyl-60,61-dioxa-53-azatricyclohexatriaconta-23,25,27(43),28(44)-tetraene-45,46,47,48,49-pentone: To a solution of (22E,24E,26E,27E,29R,30S,31R,32R,34S,36S,38S,39S,40R,41R,51R)-39-[(1R)-2-[(1S,3R,4R)-4-(difluoromethoxy)-3-methoxy-cyclohexyl]-1-methyl-ethyl]-40,51-dihydroxy-38,41-dimethoxy-29,30,31,32,42,43-hexamethyl-60,61-dioxa-52-azatricyclohexatriaconta-22,24,26(42),27(43)-tetraene-44,45,46,47,48-pentone (300 mg, 0.31 mmol) in toluene (6 mL) at rt was added N1,N1,N8,N8-tetramethylnaphthalene-1,8-diamine (867 mg, 4.04 mmol) and methyl trifluoromethanesulfonate (0.51 g, 3.11 mmol). The reaction was stirred at 50° C. for 2 h then filtered, concentrated and purified via silica gel chromatography (EtOAc:PE=1:1.5) and by reverse-phase chromatography (85% CH₃CN in water) to provide the titled compound (100 mg, 33% yield) as a white solid. ESI-MS (EI⁺, m/z): 1000.5 [M+Na]⁺.

Step 5: (25E,27E,29E,30E,33R,34S,35R,36R,38S,40S,43S,45R,46R,56R)-43-[(1R)-2-[(1S,3R,4R)-4-(difluoromethoxy)-3-methoxy-cyclohexyl]-1-methyl-ethyl]-42-(1,4-dioxan-2-ylmethoxy)-56-hydroxy-45,46-dimethoxy-33,34,35,36,47,48-hexamethyl-66,67-dioxa-57-azatricyclohexatriaconta-25,27,29(47),30(48)-tetraene-49,50,51,52,53-pentone (1-8). To a solution of (23E,25E,27E,28E,30R,31S,32R,33R,35S,37S,39S,40S,41R,42R,52R)-40-[(1R)-2-[(1S,3R,4R)-4-(difluoromethoxy)-3-methoxy-cyclohexyl]-1-methyl-ethyl]-52-hydroxy-39,41,42-trimethoxy-30,31,32,33,43,44-hexamethyl-60,61-dioxa-53-azatricyclohexatriaconta-23,25,27(43),28(44)-tetraene-45,46,47,48,49-pentone (50 mg, 0.05 mmol) and 2-(oxetan-3-yloxy)ethanol (121 mg, 1.02 mmol) in THF (5 mL) was added HND-8 (25 mg) at 50° C. under N₂. The reaction mixture was stirred for 20 h at 50° C. then cooled, concentrated and purified via silica gel chromatography (EtOAc:PE=1:1) to provide the titled compound (7 mg, 13% yield) as a white solid. ESI-MS (EI⁺, m/z): 1086.6 [M+Na]⁺, T=2.479 min. ¹H NMR (400 MHz, CDCl₃) δ 6.51-5.88 (m, 4H), 5.34 (d, J=3.5 Hz, 4H), 4.86-4.10 (m, 2H), 3.94-3.51 (m, 8H), 3.51-2.99 (m, 13H), 2.85-2.45 (m, 3H), 2.46-1.97 (m, 6H), 1.97-1.54 (m, 20H), 1.55-1.21 (m, 11H), 1.21-0.81 (m, 17H), 0.83-0.65 (m, 2H).

### Example 7: Synthesis of (3S,6R,7E,9R,10R,12R,14S,15E,17E,19E,23S,26R,27R,34aS)-27-hydroxy-3-((R)-1-((1S,3R,4R)-4-hydroxy-3-methoxycyclohexyl)propan-2-yl)-9,10-dimethoxy-6,8,12,14,20,26-hexamethyl-21-(2-(oxetan-3-yloxy)ethoxy)-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (I-11)

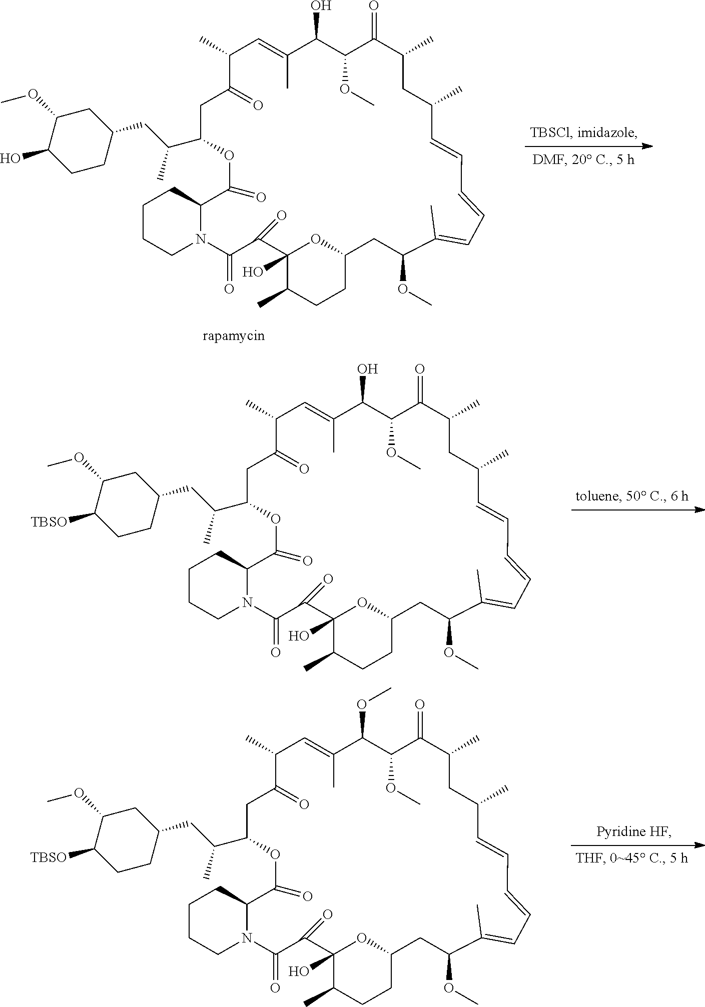

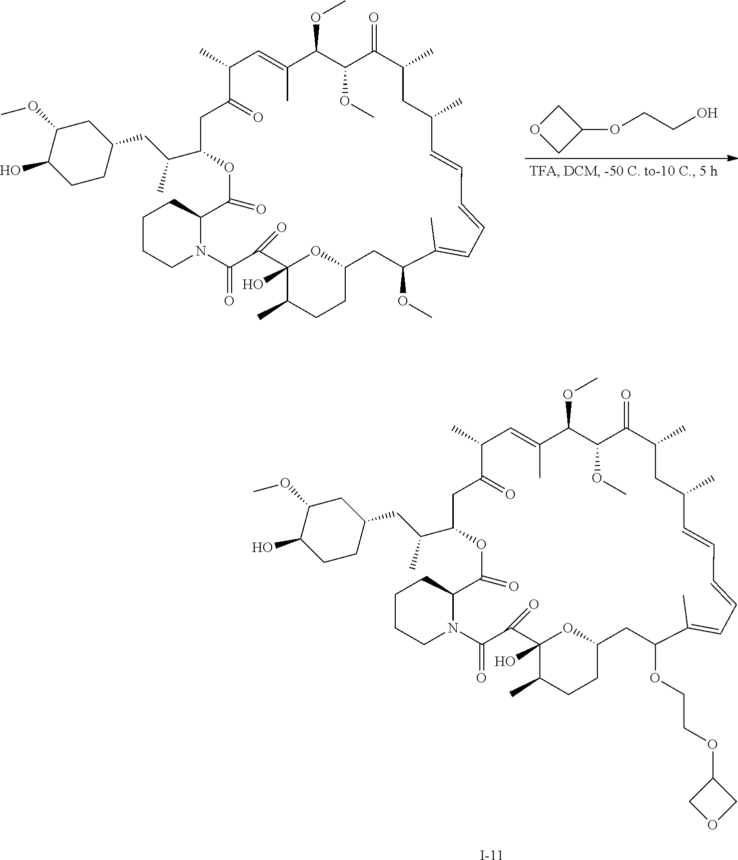

Step 1: (3S,6R,7E,9R,10R,12R,14S,15E,17E,19E,21S,23S,26R,27R,34aS)-3-((R)-1-((1S,3R,4R)-4-((tert-butyldimethylsilyl)oxy)-3-methoxycyclohexyl)propan-2-yl)-9,27-dihydroxy-10,21-dimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone. To a solution of rapamycin (5 g, 5.47 mmol) in DMF (60 mL) was added imidazole (1.49 g, 21.88 mmol) at rt, followed immediately by tert-butyl-chloro-dimethyl-silane (2.47 g, 16.41 mmol). The mixture was stirred at 50° C. for 6 h then poured into a mixture of ice cold saturated NH₄Cl aqueous solution (40 mL) and Et₂O:petroleum ether (60 mL, 2:1). The organic layer was then washed with saturated NH₄Cl aqueous solution (20 mL), washed with water and brine (20 mL), dried over Na₂SO₄, filtered and concentrated. The residue was purified via silica gel chromatography (EtOAc in petroleum ether from 10% to 50%) to provide the titled compound (4 g, 71% yield) as a white solid. ESI-MS (EI⁺, m/z): 1050.5 [M+Na]⁺.

Step 2: (3S,6R,7E,9R,10R,12R,14S,15E,17E,19E,21S,23S,26R,27R,34aS)-3-((R)-1-((1S,3R,4R)-4-((tert-butyldimethylsilyl)oxy)-3-methoxycyclohexyl)propan-2-yl)-27-hydroxy-9,10,21-trimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone. To a suspension of (3S,6R,7E,9R,10R,12R,14S,15E,17E,19E,21S,23S,26R,27R,34aS)-3-((R)-1-((1S,3R,4R)-4-((tert- butyldimethylsilyl)oxy)-3-methoxycyclohexyl)propan-2-yl)-9,27-dihydroxy-10,21-dimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (1 g, 0.97 mmol) and 1,8-bis(dimethylamino)naphtalene (2.5 g, 11.67 mmol) in toluene (15 mL) was added methyl trifluoromethanesulfonate (1.6 mL, 14.59 mmol) dropwise at rt under N₂. The mixture was then heated to 50° C. for 6 h then cooled, filtered and concentrated. The residue was purified via silica gel chromatography to provide the titled compound (0.45 g, 0.43 mmol) as a white solid. ESI-MS (EI⁺, m/z): 1064.6 [M+Na]⁺.

Step 3: (3S,6R,7E,9R,10R,12R,14S,15E,17E,19E,21S,23S,26R,27R,34aS)-27-hydroxy-3-((R)-1-((1S,3R,4R)-4-hydroxy-3-methoxycyclohexyl)propan-2-yl)-9,10,21-trimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone. To a solution of (3S,6R,7E,9R,10R,12R,14S,15E,17E,19E,21S,23S,26R,27R,34aS)-3-((R)-1-((1S,3R,4R)-4-((tert-butyldimethylsilyl)oxy)-3-methoxycyclohexyl)propan-2-yl)-27-hydroxy-9,10,21-trimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (0.4 g, 0.38 mmol) in THF (10 mL) was added pyridine hydrofluoride (3.34 mL, 38.37 mmol) at 0° C. The reaction was stirred at 45° C. for 5 h then poured into a mixture of DCM and aqueous NaHCO₃, washed with water and brine, dried over Na₂SO₄, filtered and concentrated. The residue was purified via silica gel chromatography to provide the titled compound (160 mg, 45% yield) as a white solid. ESI-MS (EI⁺, m z): 949.9 [M+Na]⁺.

Step 4: (3S,6R,7E,9R,10R,12R,14S,15E,17E,19E,23S,26R,27R,34aS)-27-hydroxy-3-((R)-1-((1S,3R,4R)-4-hydroxy-3-methoxycyclohexyl)propan-2-yl)-9,10-dimethoxy-6,8,12,14,20,26-hexamethyl-21-(2-(oxetan-3-yloxy)ethoxy)-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (I-11). To a solution of (3S,6R,7E,9R,10R,12R,14S,15E,17E,19E,21S,23S,26R,27R,34aS)-27-hydroxy-3-((R)-1-((1S,3R,4R)-4-hydroxy-3-methoxycyclohexyl)propan-2-yl)-9,10,21-trimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (500 mg, 538.68 mol) in DCM (15 mL) was added TFA (1.66 mL, 21.55 mmol) at −50° C. The mixture was stirred at the same temperature for 10 minutes then 2-(oxetan-3-yloxy) ethanol (1.91 g, 16.16 mmol) dissolved in DCM (0.2 mL) was added and the mixture was stirred at −10° C. for 5 h. The reaction was diluted with a mixture of DCM and aqueous NaHCO₃, washed with water and brine, dried over Na₂SO₄, filtered and concentrated. The residue was then purified via reverse phase chromatography (70% CH₃CN in water) to provide I-11. ESI-MS (EI⁺, m/z): 1036.3 [M+Na]⁺. ¹H NMR (400 MHz, CDCl₃) δ 6.51-5.80 (m, 4H), 5.75-5.03 (m, 4H), 4.83-4.09 (m, 4H), 3.99-3.53 (m, 7H), 3.52-3.02 (m, 15H), 3.01-2.44 (m, 5H), 2.11 (ddd, J=99.8, 49.8, 39.7 Hz, 7H), 1.83-1.61 (m, 13H), 1.52-1.20 (m, 10H), 1.18-0.80 (m, 17H), 0.69 (dd, J=23.8, 11.9 Hz, 1H).

### Example 8: Synthesis of (28E,30E,32E,33E,39R,40S,41R,42R,44S,46S,49S,51R,52R,61R)-48-(1,4-dioxan-2-ylmethoxy)-51,61-dihydroxy-52-methoxy-49-[(1R)-2-[(1S,3R,4R)-3-methoxy-4-(3-morpholinopropoxy)cyclohexyl]-1-methyl-ethyl]-39,40,41,42,53,54-hexamethyl-74,75-dioxa-63-azatricyclohexatriaconta-28,30,32(53),33(54)-tetraene-55,56,57,58,59-pentone (I-12)

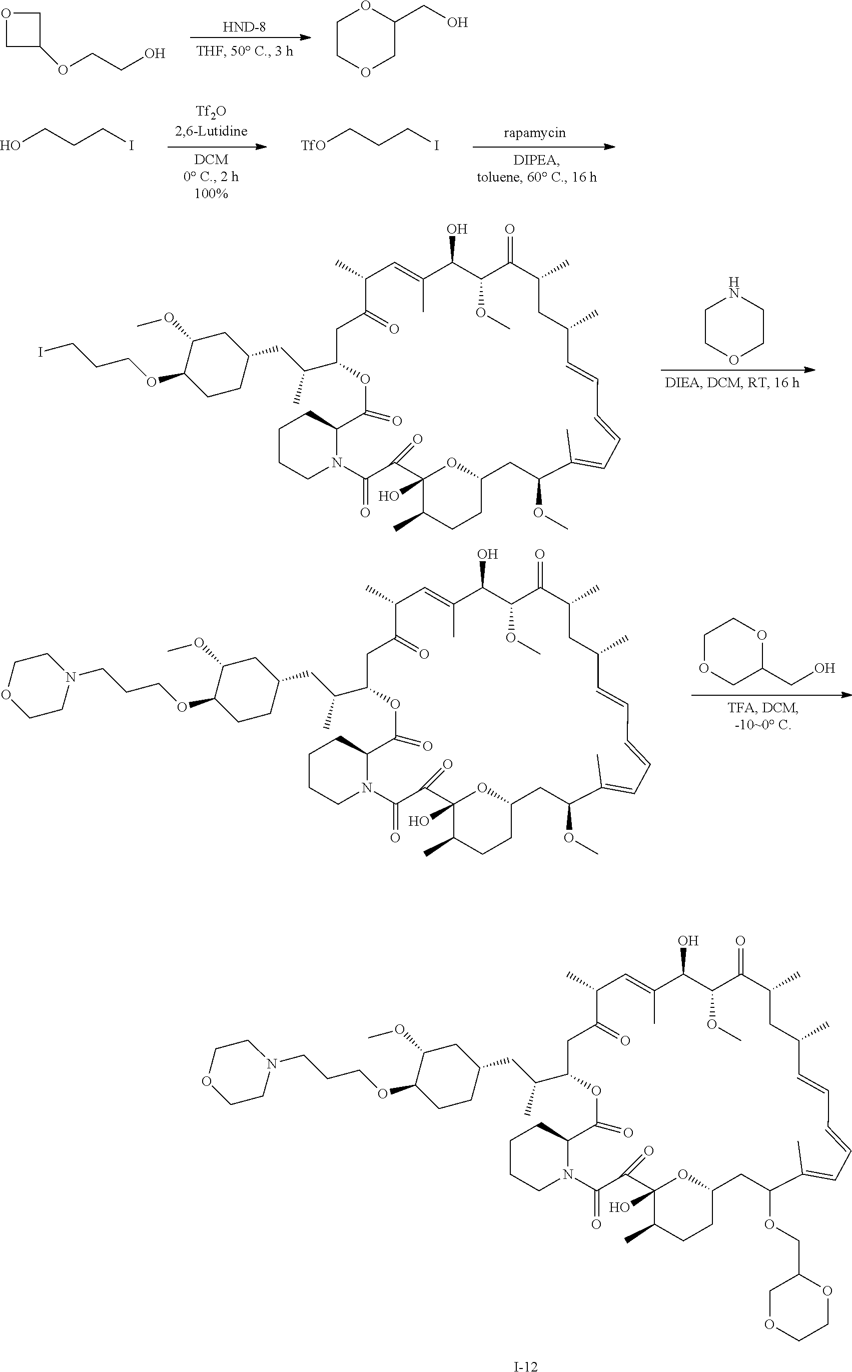

Step 1: 1, 4-dioxan-2-ylmethanol. A mixture of 2-(oxetan-3-yloxy)ethanol (7.77 g, 65.77 mmol) and HND-8 (2.33 g, 65.77 mmol) in THF (120 mL) was stirred at 50° C. for 3 h. The mixture was filtered and concentrated to provide 1,4-dioxan-2-ylmethanol (6.97 g, 90% yield) as a colorless oil. ¹H NMR (400 MHz, CDCl₃) δ 3.87-3.39 (m, 9H), 2.37-2.11 (m, 1H).

Step 2: 3-iodopropyltrifluoromethanesulfonate. A mixture of 3-iodopropan-1-ol (4 g, 21.51 mmol) and 2,6-lutidine (4.61 g, 43.01 mmol) in DCM (40 mL) was cooled to 0° C. under N₂, and trifluoromethylsulfonyl trifluoromethanesulfonate (6.67 g, 23.66 mmol) was added dropwise. The resulting solution was stirred at 0° C. for 2 h then quenched with 10% EtOAc in petroleum ether and passed through a short silica gel column, filtered and concentrated to afford 3-iodopropyl trifluoromethanesulfonate (6.72 g, 98% yield).

Step 3: (22E,24E,26E,27E,32R,33S,34R,35R,37S,39S,41S,42S,43R,44R,53R)-43,53-dihydroxy-42-[(1R)-2-[(1S,3R,4R)-4-(3-iodopropoxy)-3-methoxy-cyclohexyl]-1-methyl-ethyl]-41,44-dimethoxy-32,33,34,35,45,46-hexamethyl-62,63-dioxa-54-azatricyclohexatriaconta-22,24,26(45),27(46)-tetraene-47,48,49,50,51-pentone. A mixture of rapamycin (2 g, 2.19 mmol) and N-ethyl-N-isopropyl-propan-2-amine (4.24 g, 32.82 mmol) in toluene (40 mL) was stirred at 50° C. for 16 h. The mixture was poured into ice cold saturated NaHCO₃ (50 mL), washed with ice-water twice (60 mL), brine (50 mL), and then dried over anhydrous Na₂SO₄, filtered and concentrated. The residue was purified via silica gel chromatography (petroleum ether:EA=3:1) to provide the titled compound (1.45 g, 61% yield) as a light-yellow solid. ESI-MS (EI⁺, m/z): 1104.5 [M+Na]⁺.

Step 4: (26E,28E,30E,31E,36R,37S,38R,39R,41S,43S,45S,46S,47R,48R,57R)-47,57-dihydroxy-45,48-dimethoxy-46-[(1R)-2-[(1S,3R,4R)-3-methoxy-4-(3-morpholinopropoxy)cyclohexyl]-1-methyl-ethyl]-36,37,38,39,49,50-hexamethyl-68,69-dioxa-59-azatricyclohexatriaconta-26,28,30(49),31(50)-tetraene-51,52,53,54,55-pentone. A mixture of (22E,24E,26E,27E,32R,33S,34R,35R,37S,39S,41S,42S,43R,44R,53R)-43,53-dihydroxy-42-[(1R)-2-[(1S,3R,4R)-4-(3-iodopropoxy)-3-methoxy-cyclohexyl]-1-methyl-ethyl]-41,44-dimethoxy-32,33,34,35,45,46-hexamethyl-62,63-dioxa-54-azatricyclohexatriaconta-22,24,26(45),27(46)-tetraene-47,48,49,50,51-pentone (1.35 g, 1.25 mmol) and N-ethyl-N-isopropyl-propan-2-amine (0.48 g, 3.74 mmol) in DCM (7.2 mL) was stirred at rt for 16 h. The reaction mixture was diluted with DCM and acidified with HCl 1N to pH 5. The organic phase was washed with H₂O, passed through a phase separator then dried over anhydrous Na₂SO₄ and concentrated. The residue was purified via silica gel chromatography (EA:5% NH₃/MeOH) to provide the titled compound (0.5 g, 37% yield) as a light-yellow solid. ESI-MS (EI+, m/z): 1042.0 [M+Na]⁺.

Step 5: (28E,30E,32E,33E,39R,40S,41R,42R,44S,46S,49S,51R,52R,61R)-48-(1,4-dioxan-2-ylmethoxy)-51,61-dihydroxy-52-methoxy-49-[(1R)-2-[(1S,3R,4R)-3-methoxy-4-(3-morpholinopropoxy)cyclohexyl]-1-methyl-ethyl]-39,40,41,42,53,54-hexamethyl-74,75-dioxa-63-azatricyclohexatriaconta-28,30,32(53),33(54)-tetraene-55,56,57,58,59-pentone (I-12). To a solution of (26E,28E,30E,31E,36R,37S,38R,39R,41S,43S,45S,46S,47R,48R,57R)-47,57-dihydroxy-45,48-dimethoxy-46-[(1R)-2-[(1S,3R,4R)-3-methoxy-4-(3-morpholinopropoxy)cyclohexyl]-1-methyl-ethyl]-36,37,38,39,49,50-hexamethyl-68,69-dioxa-59-azatricyclohexatriaconta-26,28,30(49),31(50)-tetraene-51,52,53,54,55-pentone (0.4 g, 0.38 mmol) and 1,4-dioxan-2-ylmethanol (1.36 g, 11.52 mmol) in DCM (16 mL) was added 2,2,2-trifluoroacetic acid (1.75 g, 15.36 mmol) at 0° C. under N₂. The reaction mixture was stirred for 20 h at −10° C. then washed with ice saturated aqueous NaHCO₃ (10 mL), water (10 mL×3) and brine (10 mL×3), dried over anhydrous sodium sulfate, filtered and concentrated. The reaction mixture was purified by reverse phase chromatography eluting with 50% CH₃CN in water to provide I-12 (156 mg, 34% yield). ESI-MS (EI⁺, m/z): 1150.7 [M+Na]⁺. ¹H NMR (400 MHz, CDCl₃) δ 6.51-6.01 (m, 4H), 5.47 (d, J=43.7 Hz, 3H), 5.33-5.10 (m, 2H), 4.22 (d, J=31.5 Hz, 2H), 3.73 (dd, J=48.2, 40.9 Hz, 12H), 3.39 (dd, J=28.6, 10.3 Hz, 10H), 3.02 (d, J=10.8 Hz, 3H), 2.71 (d, J=16.9 Hz, 9H), 2.32 (s, 2H), 2.12-1.37 (m, 31H), 1.35-0.75 (m, 20H).

### Example 9: Synthesis of (27E,29E,31E,32E,39R,40S,41R,42R,44S,46S,48S,49S,51R,52R,61R)-51,61-dihydroxy-52-methoxy-49-[(1R)-2-[(1S,3R,4R)-3-methoxy-4-(3-morpholinopropoxy)cyclohexyl]-1-methyl-ethyl]-39,40,41,42,53,54-hexamethyl-48-[2-(oxetan-3-yloxy)ethoxy]-73,74-dioxa-63-azatricyclohexatriaconta-27,29,31(53),32(54)-tetraene-55,56,57,58,59-pentone (I-14) and (27E,29E,31E,32E,39R,40S,41R,42R,44S,46S,48R,49S,51R,52R,61R)-51,61-dihydroxy-52-methoxy-49-[(1R)-2-[(1S,3R,4R)-3-methoxy-4-(3-morpholinopropoxy)cyclohexyl]-1-methyl-ethyl]-39,40,41,42,53,54-hexamethyl-48-[2-(oxetan-3-yloxy)ethoxy]-73,74-dioxa-63-azatricyclohexatriaconta-27,29,31(53),32(54)-tetraene-55,56,57,58,59-pentone (I-13)

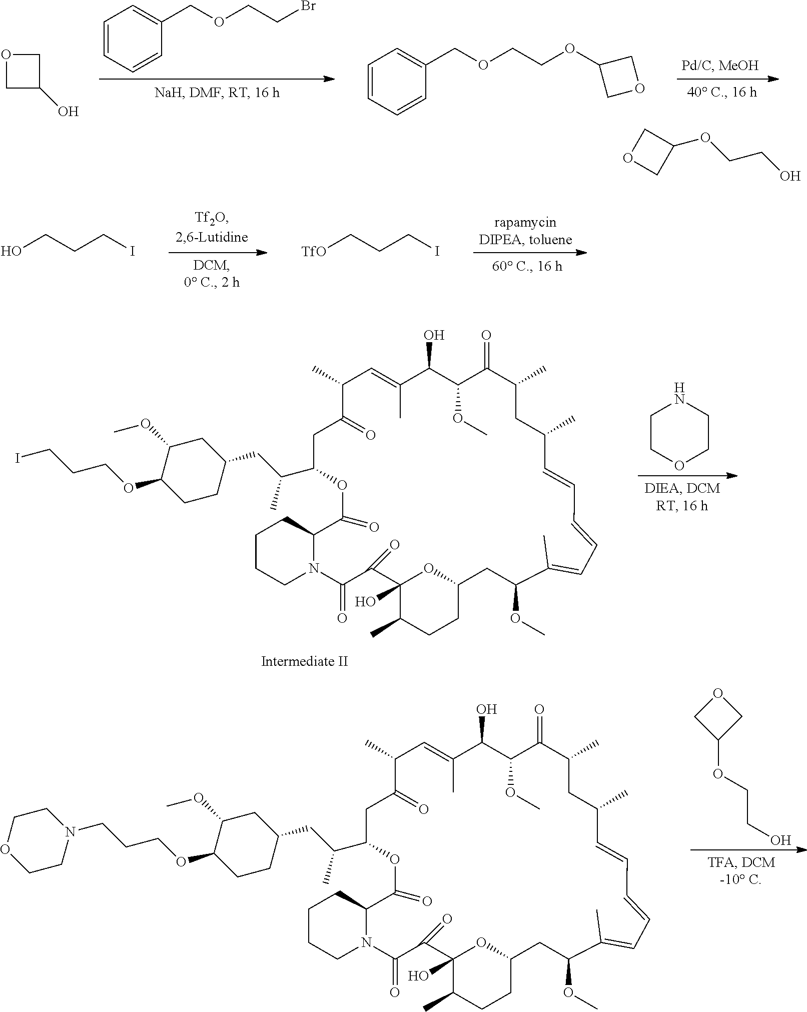

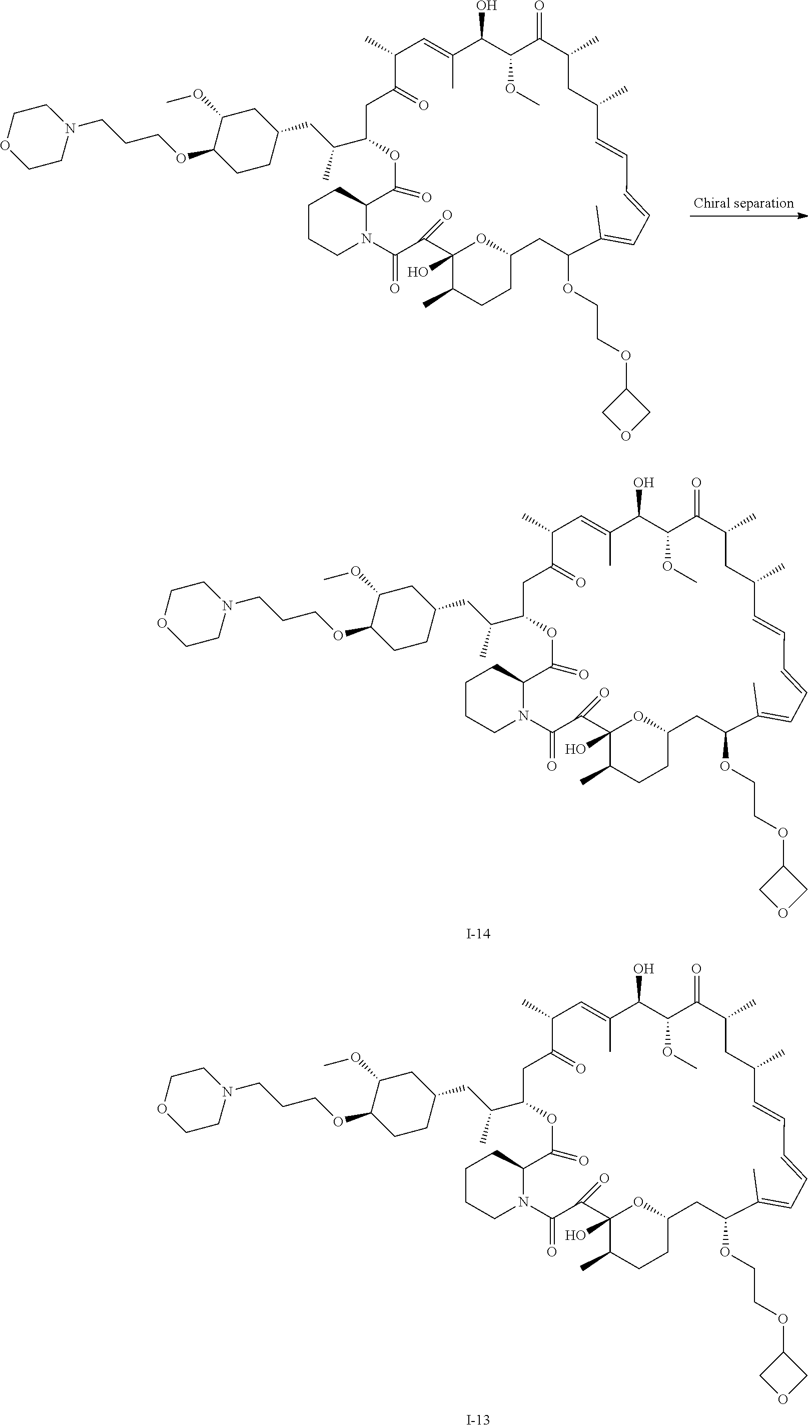

Step 1: 3-(2-benzyloxyethoxy) oxetane. To a solution of oxetan-3-ol (10 g, 135 mmol) in DMF (160 mL) was added sodium hydride (3.24 g, 135 mmol) at 0° C. The resulting solution was stirred at this temperature for 30 min then 2-bromoethoxymethylbenzene (43.55 g, 202.49 mmol) was added. The resulting solution was stirred for 2 h at 0° C. then for 16 h at room temperature. The reaction was quenched by the addition of 800 mL of NH₄Cl (sat., aq.), then extracted with 2×120 mL of ethyl acetate and the organic layers combined and concentrated. The residue was purified via silica gel chromatography eluting with petroleum ether/EA (8:1) to provide the titled compound (16.4 g, 78.75 mmol) as a colorless liquid. ESI-MS (EI⁺, m/z): 231 [M+Na]⁺. ¹H NMR (400 MHz, CDCl₃) δ 7.41-7.23 (m, 6H), 4.79-4.70 (m, 2H), 4.68-4.52 (m, 6H), 3.62-3.53 (m, 4H).

Step 2: 2-(oxetan-3-yloxy) ethanol. To a solution of 3-(2-benzyloxyethoxy) oxetane (4 g, 19.21 mmol) in MeOH (20 mL) was added Pd/C (2.04 g, 19.21 mmol) under N₂, then the resulting solution was stirred under H₂ at 40° C. overnight, then filtered and concentrated. The residue was purified via silica gel chromatography eluting with petroleum ether:EA=1:5 to afford the titled compound (2.1 g, 93% yield) as a colorless liquid. ¹H NMR (400 MHz, CDCl₃) δ 4.79 (td, J=5.8, 2.1 Hz, 2H), 4.62 (dt, J=10.2, 4.9 Hz, 3H), 3.80-3.69 (m, 2H), 3.52-3.44 (m, 2H), 2.36 (s, 1H).

Step 3: 3-iodopropyltrifluoromethanesulfonate. To a mixture of 3-iodopropan-1-ol (4 g, 21.5 mmol) and 2,6-lutidine (4.61 g, 43 mmol) in DCM (40 mL) at 0° C. under N₂, was added trifluoromethylsulfonyl trifluoromethanesulfonate (6.67 g, 23.66 mmol) dropwise. The resulting solution was stirred at 0° C. for 2 h then quenched with 10% EtOAc in petroleum ether and filtered through a short silica gel column; the filtrate was concentrated in vacuo to afford the titled compound (6.72 g, 98% yield) as a light yellow liquid.

Step 4: (22E,24E,26E,27E,32R,33S,34R,35R,37S,39S,41S,42S,43R,44R,53R)-43,53-dihydroxy-42-[(1R)-2-[(1S,3R,4R)-4-(3-iodopropoxy)-3-methoxy-cyclohexyl]-1-methyl-ethyl]-41,44-dimethoxy-32,33,34,35,45,46-hexamethyl-62,63-dioxa-54-azatricyclohexatriaconta-22,24,26(45),27(46)-tetraene-47,48,49,50,51-pentone. A mixture of rapamycin (2 g, 2.19 mmol) and N-ethyl-N-isopropyl-propan-2-amine (5.72 mL, 32.82 mmol) in toluene (40 mL) was stirred at 50° C. for 16 h, then poured into ice cold saturated NaHCO₃ (50 mL), washed with ice-water (60 mL×2), brine (50 mL), dried over anhydrous Na₂SO₄, filtered and concentrated. The residue was purified via silica gel chromatography (petroleum ether:EA=3:1) to provide the titled compound (1.45 g, 61% yield) as a light-yellow solid. ESI-MS (EI⁺, m/z): 1104.5 [M+Na]⁺.

Step 5: (26E,28E,30E,31E,36R,37S,38R,39R,41S,43S,45S,46S,47R,48R,57R)-47,57-dihydroxy-45,48-dimethoxy-46-[(1R)-2-[(1S,3R,4R)-3-methoxy-4-(3-morpholinopropoxy)cyclohexyl]-1-methyl-ethyl]-36,37,38,39,49,50-hexamethyl-68,69-dioxa-59-azatricyclohexatriaconta-26,28,30(49),31(50)-tetraene-51,52,53,54,55-pentone. A mixture of (22E,24E,26E,27E,32R,33S,34R,35R,37S,39S,41S,42S,43R,44R,53R)-43,53-dihydroxy-42-[(1R)-2-[(1S,3R,4R)-4-(3-iodopropoxy)-3-methoxy-cyclohexyl]-1-methyl-ethyl]-41,44-dimethoxy-32,33,34,35,45,46-hexamethyl-62,63-dioxa-54-azatricyclohexatriaconta-22,24,26(45),27(46)-tetraene-47,48,49,50,51-pentone (Intermediate II, 1.35 g, 1.25 mmol) and N-ethyl-N-isopropyl-propan-2-amine (0.65 mL, 3.74 mmol) in DCM (7.2 mL) was stirred at rt for 16 h. The reaction mixture was diluted with DCM and acidified with 1N HCl aqueous solution to pH=5. The organic phase was washed with H₂O, filtered through a phase separator, dried over anhydrous Na₂SO₄, filtered and concentrated. The residue was purified via silica gel chromatography (EA: 5% 7 M NH₃ in MeOH=4:1) to provide the titled compound (498 mg, 37% yield) as a light-yellow solid. ESI-MS (EI⁺, m/z): 1042.0 [M+Na]⁺.

Step 6: (27E,29E,31E,32E,39R,40S,41R,42R,44S,46S,49S,51R,52R,61R)-51,61-dihydroxy-52-methoxy-49-[(1R)-2-[(1S,3R,4R)-3-methoxy-4-(3-morpholinopropoxy)cyclohexyl]-1-methyl-ethyl]-39,40,41,42,53,54-hexamethyl-48-[2-(oxetan-3-yloxy)ethoxy]-73,74-dioxa-63-azatricyclohexatriaconta-27,29,31(53),32(54)-tetraene-55,56,57,58,59-pentone. To a solution of (26E,28E,30E,31E,37R,38S,39R,40R,42S,44S,46S,47S,48R,49S,58R)-48-ethyl-49,58-dihydroxy-46-methoxy-47-[(1R)-2-[(1S,3R,4R)-3-methoxy-4-(3-morpholinopropoxy)cyclohexyl]-1-methyl-ethyl]-37,38,39,40,50,51-hexamethyl-69,70-dioxa-60-azatricyclohexatriaconta-26,28,30(50),31(51)-tetraene-52,53,54,55,56-pentone (200 mg, 0.19 mmol) in DCM (30 mL) was added 2,2,2-trifluoroacetic acid (0.59 mL, 7.70 mmol) dropwise at −50° C. under N₂. After addition, the reaction mixture was stirred for 10 min at −50° C. then 2-(oxetan-3-yloxy) ethanol (682 mg, 5.77 mmol, dissolved in DCM) was added to the reaction mixture at the same temperature. The reaction mixture was stirred for 2 h at −10° C. then poured into saturated aqueous NaHCO₃ (15 mL) at 0° C. and extracted with DCM (20 mL). The organic layer was washed with water (50 mL) and brine (50 mL), dried over anhydrous sodium sulfate, filtered and concentrated. The residue was purified via reverse phase chromatography eluting with 50% CH₃CN in water to provide the titled compound (40 mg, 18% yield) as a white solid. ESI-MS (EI⁺, m/z): 1126.69 [M+H]⁺. ¹H NMR (400 MHz, CDCl₃) δ 6.39-5.98 (m, 4H), 5.55-5.03 (m, 5H), 4.78-4.43 (m, 4H), 4.15 (d, J=40.7 Hz, 2H), 3.71 (t, J=21.3 Hz, 6H), 3.60-3.45 (m, 3H), 3.46-3.14 (m, 10H), 2.96 (d, J=11.0 Hz, 3H), 2.56 (d, J=54.3 Hz, 8H), 2.26 (s, 2H), 2.17-2.03 (m, 2H), 1.94 (s, 4H), 1.80-1.32 (m, 15H), 1.28-1.10 (m, 11H), 1.06-0.56 (m, 19H).

Step 7: (27E,29E,31E,32E,39R,40S,41R,42R,44S,46S,48S,49S,51R,52R,61R)-51,61-dihydroxy-52-methoxy-49-[(1R)-2-[(1S,3R,4R)-3-methoxy-4-(3-morpholinopropoxy)cyclohexyl]-1-methyl-ethyl]-39,40,41,42,53,54-hexamethyl-48-[2-(oxetan-3-yloxy)ethoxy]-73,74-dioxa-63-azatricyclohexatriaconta-27,29,31(53),32(54)-tetraene-55,56,57,58,59-pentone (I-14) and (27E,29E,31E,32E,39R,40S,41R,42R,44S,46S,48R,49S,51R,52R,61R)-51,61-dihydroxy-52-methoxy-49-[(1R)-2-[(1S,3R,4R)-3-methoxy-4-(3-morpholinopropoxy)cyclohexyl]-1-methyl-ethyl]-39,40,41,42,53,54-hexamethyl-48-[2-(oxetan-3-yloxy)ethoxy]-73,74-dioxa-63-azatricyclohexatriaconta-27,29,31(53),32(54)-tetraene-55,56,57,58,59-pentone (I-13). 120 mg of (27E,29E,31E,32E,39R,40S,41R,42R,44S,46S,49S,51R,52R,61R)-51,61-dihydroxy-52-methoxy-49-[(1R)-2-[(1S,3R,4R)-3-methoxy-4-(3-morpholinopropoxy)cyclohexyl]-1-methyl-ethyl]-39,40,41,42,53,54-hexamethyl-48-[2-(oxetan-3-yloxy)ethoxy]-73,74-dioxa-63-azatricyclohexatriaconta-27,29,31(53),32(54)-tetraene-55,56,57,58,59-pentone was separated via chiral preparative HPLC then purified via silica gel chromatography (17% MeOH in petroleum ether:DCM:EA: 3:3:1) to provide I-14 (7.2 mg, 6% yield) as a white solid and I-13 (14.8 mg, 12% yield) as a white solid.

Chiral separation method:

Column: CHIRALPAK IC

Column size: 2.5 cm I.D.×25 cm L, 10 μm

Sample solution: 1.3 mg/mL in mobile phase

Injection: 8 mL

Mobile phase: Hexane/EtOH=50/50(V/V)

Flow rate: 20 mL/min

Wave length: UV 254 nm

Temperature: 38° C.

I-14: ESI-MS (EI⁺, m/z): 1126.8 [M+H]⁺; 1148.9 [M+Na]⁺. ¹H NMR (500 MHz, CDCl₃) δ 6.41-5.74 (m, 4H), 5.51-5.01 (m, 4H), 4.80-4.41 (m, 5H), 4.10 (d, J=5.4 Hz, 1H), 3.88-2.88 (m, 26H), 2.72-2.13 (m, 12H), 2.00-1.27 (m, 26H), 1.10-0.59 (m, 20H).
I-13: ESI-MS (EI⁺, m/z): 1126.8 [M+H]⁺; 1148.9 [M+Na]⁺.

¹H NMR (500 MHz, CDCl₃) δ 6.41-5.80 (m, 4H), 5.63-5.01 (m, 4H), 4.76-4.45 (m, 5H), 4.25-3.92 (m, 2H), 3.87-2.88 (m, 25H), 2.71-2.04 (m, 12H), 1.91 (d, J=28.3 Hz, 5H), 1.62 (ddt, J=39.3, 32.9, 10.5 Hz, 14H), 1.47-1.29 (m, 7H), 1.08-0.55 (m, 20H).

### Example 10: Synthesis of (28E,30E,32E,33E,40R,41S,42R,43R,45S,47S,50S,52R,53R,62R)-52,62-dihydroxy-53-methoxy-50-[(1R)-2-[(1S,3R,4R)-3-methoxy-4-[3-(4-methylpiperazin-1-yl)propoxy]cyclohexyl]-1-methyl-ethyl]-40,41,42,43,54,55-hexamethyl-49-[2-(oxetan-3-yloxy)ethoxy]-74,75-dioxa-65-azatricyclohexatriaconta-28,30,32(54),33(55)-tetraene-56,57,58,59,60-pentone (I-15)

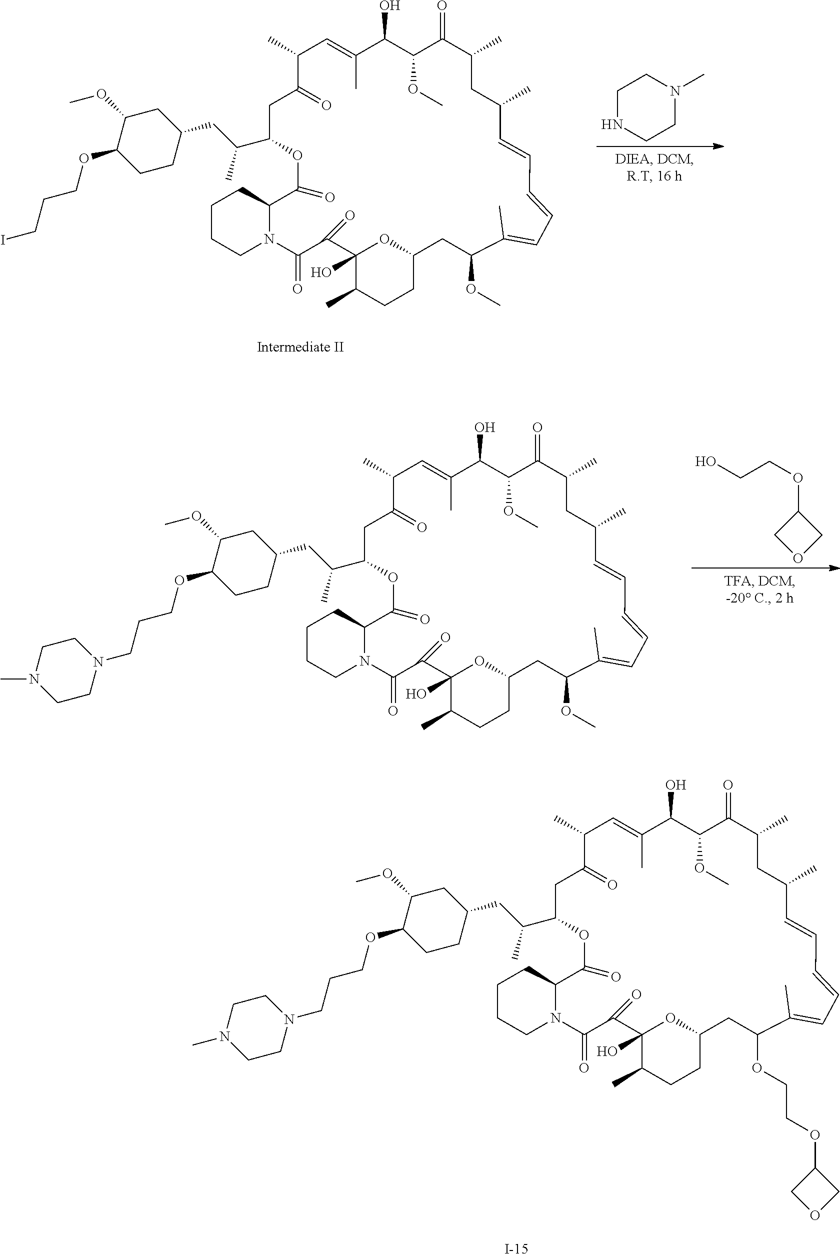

Step 1: (27E,29E,31E,32E,37R,38S,39R,40R,42S,44S,46S,47S,48R,49R,58R)-48,58-dihydroxy-46,49-dimethoxy-47-[(1R)-2-[(1S,3R,4R)-3-methoxy-4-[3-(4-methylpiperazin-1-yl)propoxy]cyclohexyl]-1-methyl-ethyl]-37,38,39,40,50,51-hexamethyl-69,70-dioxa-61-azatricyclohexatriaconta-27,29,31(50),32(51)-tetraene-52,53,54,55,56-pentone. A mixture of (22E,24E,26E,27E,32R,33S,34R,35R,37S,39S,41S,42S,43R,44R,53R)-43,53-dihydroxy-42-[(1R)-2-[(1S,3R,4R)-4-(3-iodopropoxy)-3-methoxy-cyclohexyl]-1-methyl-ethyl]-41,44-dimethoxy-32,33,34,35,45,46-hexamethyl-62,63-dioxa-54-azatricyclohexatriaconta-22,24,26(45),27(46)-tetraene-47,48,49,50,51-pentone (Intermediate II, 1.45 g, 1.34 mmol), 1-methylpiperazine (0.16 g, 1.61 mmol) and N-ethyl-N-isopropyl-propan-2-amine (0.52 g, 4.02 mmol) in DCM (30 mL) was stirred at rt for 16 h then diluted with DCM and acidified with 1N HCl to pH 5. The organic phase was washed with H₂O, passed through a phase separator then dried over anhydrous Na₂SO₄ and concentrated. The residue was purified via silica gel chromatography (EA:5% NH₃/MeOH) to provide the titled compound (0.96 g, 68% yield) as a light-yellow solid. ¹H NMR (400 MHz, CDCl₃): δ 5.95-6.39 (m, 4H), 5.16-5.55 (m, 4H), 4.10-4.22 (m, 2H), 3.55-3.87 (m, 6H), 3.30-3.43 (m, 8H), 2.98-3.17 (m, 6H), 2.67-2.86 (m, 9H), 2.50-2.64 (m, 3H), 2.46 (S, 2H), 2.27-2.35 (m, 2H), 1.95-2.05 (m, 7H), 1.79-1.86 (m, 3H), 1.74-1.76 (m, 3H), 1.58-1.71 (m, 8H), 1.46-1.54 (m, 3H), 1.31-1.35 (m, 2H), 0.86-1.35 (m, 23H).

Step 2: (28E,30E,32E,33E,40R,41S,42R,43R,45S,47S,50S,52R,53R,62R)-52,62-dihydroxy-53-methoxy-50-[(1R)-2-[(1S,3R,4R)-3-methoxy-4-[3-(4-methylpiperazin-1-yl)propoxy]cyclohexyl]-1-methyl-ethyl]-40,41,42,43,54,55-hexamethyl-49-[2-(oxetan-3-yloxy)ethoxy]-74,75-dioxa-65-azatricyclohexatriaconta-28,30,32(54),33(55)-tetraene-56,57,58,59,60-pentone (I-15). To a solution of (27E,29E,31E,32E,37R,38S,39R,40R,42S,44S,46S,47S,48R,49R,58R)-48,58-dihydroxy-46,49-dimethoxy-47-[(1R)-2-[(1S,3R,4R)-3-methoxy-4-[3-(4-methylpiperazin-1-yl)propoxy]cyclohexyl]-1-methyl-ethyl]-37,38,39,40,50,51-hexamethyl-69,70-dioxa-61-azatricyclohexatriaconta-27,29,31(50),32(51)-tetraene-52,53,54,55,56-pentone (0.78 g, 0.74 mmol) in DCM (20 mL) was added 2,2,2-trifluoroacetic acid (3.38 g, 29.65 mmol) dropwise at −40° C. under N₂. After addition, the reaction mixture was stirred for 10 min at −40° C. then 2-(oxetan-3-yloxy) ethanol (2.63 g, 22.23 mmol in DCM) was added to the reaction mixture at the same temperature. The reaction mixture was stirred for 2 h at −20° C. then poured into saturated aqueous NaHCO₃ solution (25 mL) at 0° C. and extracted with DCM (25 mL). The organic layer was washed with water (25 mL) and brine (25 mL), dried over anhydrous sodium sulfate, filtered and the filtrate was concentrated under vacuum. The residue was purified by reverse phase chromatography eluting with 50% CH₃CN in 0.01% HCOOH in water to provide I-15 (120 mg, 14% yield) as a white solid. ESI-MS (EI⁺, m/z): 1140.8 [M+Na]⁺. ¹H NMR (400 MHz, CDCl₃): δ 5.97-6.36 (m, 4H), 5.15-5.49 (m, 4H), 4.57-4.79 (m, 5H), 4.04-4.27 (m, 2H), 3.73-3.86 (m, 2H), 3.54-3.64 (m, 3H), 3.33-3.52 (m, 11H), 2.64-3.09 (m, 15H), 2.48-2.63 (m, 4H), 2.28-2.35 (m, 2H), 1.86-2.11 (m, 8H), 1.61-1.79 (m, 11H), 1.14-1.56 (m, 12H), 0.82-1.09 (m, 18H).

### Example 11: Synthesis of [(40S,42R,44R)-4-[(2R)-2-[(27E,29E,31E,32E,36R,37S,38R,39R,41S,43S,45S,46S,47R,48R,58R)-47,58-dihydroxy-45,48-dimethoxy-36,37,38,39,49,50-hexamethyl-51,52,53,54,55-pentaoxo-71,72-dioxa-60-azatricyclohexatriaconta-27,29,31(49),32(50)-tetraen-46-yl]propyl]-44-methoxy-42-cyclohexyl]N-methyl-N-(2-morpholinoethyl)carbamate (I-16), [(43S,45R,47R)-4-[(2R)-2-[(28E,30E,32E,33E,39R,40S,41R,42R,44S,46S,48S,49S,51R,52R,62R)-51,62-dihydroxy-52-methoxy-39,40,41,42,53,54-hexamethyl-48-[2-(oxetan-3-yloxy)ethoxy]-55,56,57,58,59-pentaoxo-76,77-dioxa-64-azatricyclohexatriaconta-28,30,32(53),33(54)-tetraen-49-yl]propyl]-47-methoxy-45-cyclohexyl] N-methyl-N-(2-morpholinoethyl)carbamate (I-18) and [(43S,45R,47R)-4-[(2R)-2-[(28E,30E,32E,33E,39R,40S,41R,42R,44S,46S,48R,49S,51R,52R,62R)-51,62-dihydroxy-52-methoxy-39,40,41,42,53,54-hexamethyl-48-[2-(oxetan-3-yloxy)ethoxy]-55,56,57,58,59-pentaoxo-76,77-dioxa-64-azatricyclohexatriaconta-28,30,32(53),33(54)-tetraen-49-yl]propyl]-47-methoxy-45-cyclohexyl] N-methyl-N-(2-morpholinoethyl)carbamate (I-17)

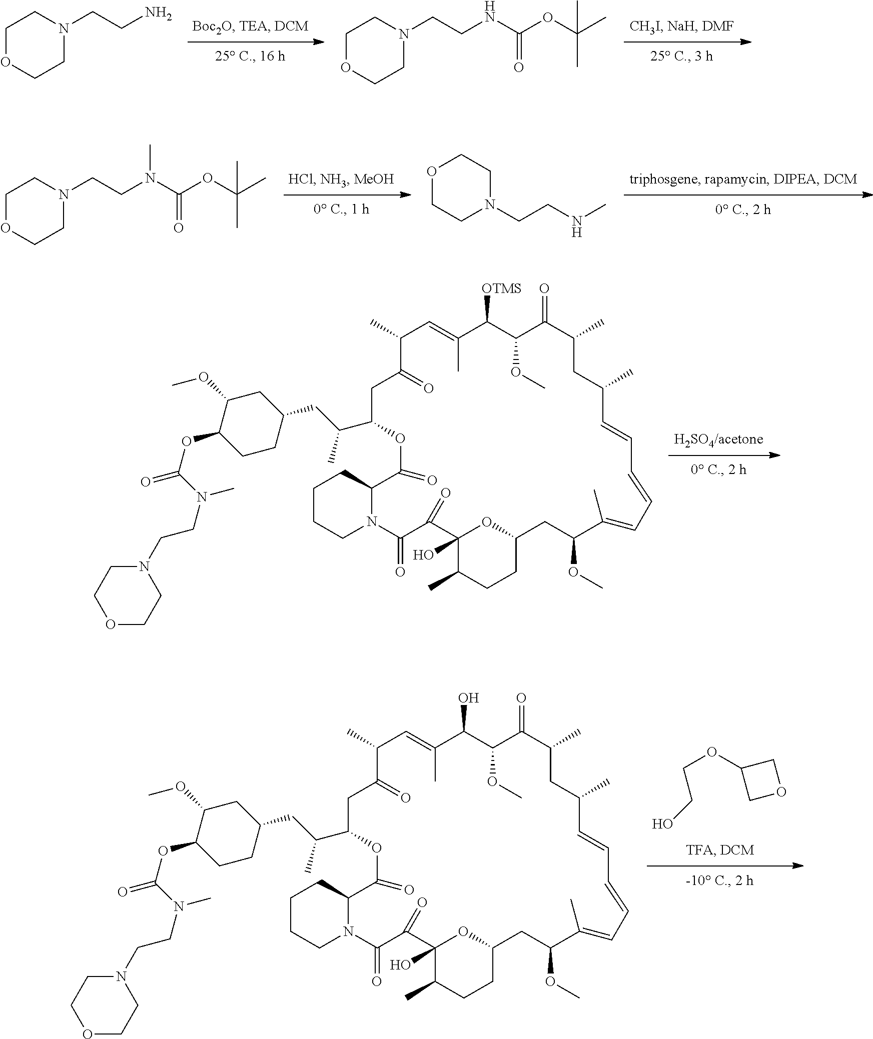

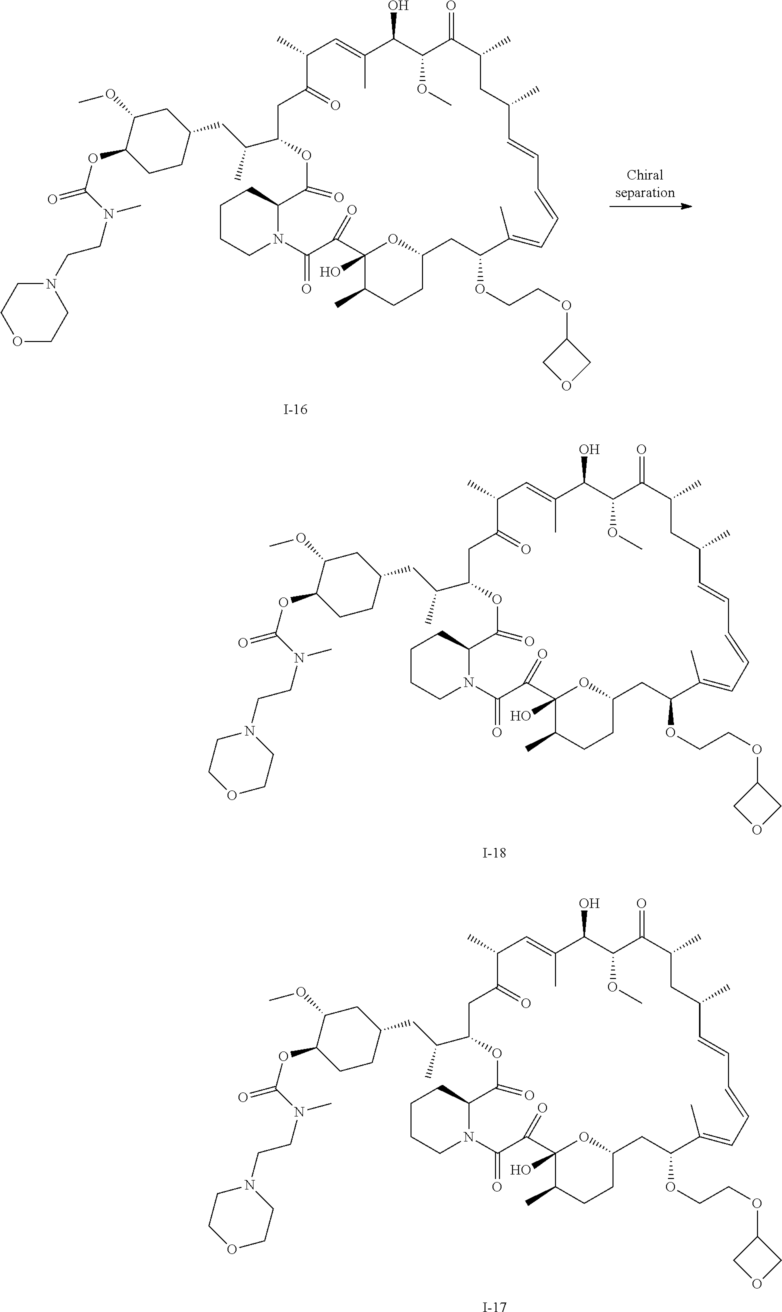

Step 1: tert-butyl N-(2-morpholinoethyl) carbamate. To a solution of 2-morpholinoethanamine (10 g, 76.81 mmol) in DCM (5 mL) was added triethylamine (5.35 mL, 38.41 mmol) and tert-butoxycarbonyl tert-butyl carbonate (18.44 g, 84.5 mmol) at 0° C. and the resulting solution was stirred overnight at 25° C. The reaction mixture was diluted with 200 mL of dichloromethane and then washed with 30 mL of 10% sodium bicarbonate and 30 mL of brine. The organic layer was dried over sodium sulfate, filtered and concentrated to provide the titled compound (17 g, 96% yield) as an off-white solid. ESI-MS (EI⁺, m/z): 231.3 [M+H]⁺. ¹H NMR (400 MHz, CDCl₃) δ 3.78-3.62 (m, 4H), 3.24 (d, J=5.5 Hz, 2H), 2.45 (dd, J=8.0, 3.9 Hz, 6H), 1.49-1.42 (m, 9H).

Step 2: tert-butyl N-methyl-N-(2-morpholinoethyl) carbamate. Tert-butyl N-(2-morpholinoethyl)carbamate (18 g, 78.16 mmol) was dissolved in DMF (240 mL) cooled to 0° C. and NaH (9.38 g, 234.47 mmol, 60% purity) was added. The reaction was stirred at room temperature for 20 minutes then cooled to 0° C., iodomethane (12.2 g, 85.97 mmol) was added, and the mixture stirred for a further 3 h. The reaction was then diluted with ethyl acetate (500 ml) and washed sequentially with saturated aqueous ammonium chloride solution (300 mL) and brine (300 mL×5). The organic layer was dried over sodium sulfate, filtered, and concentrated under reduced pressure to provide the crude titled compound (14 g, 73% yield) as a white solid. ESI-MS (EI⁺, m/z): 245.3 [M+H]⁺. ¹H NMR (400 MHz, CDCl₃) δ 3.74-3.64 (m, 4H), 3.34 (s, 2H), 2.93-2.81 (m, 3H), 2.48 (d, J=4.8 Hz, 6H), 1.46 (s, 10H).

Step 3: N-methyl-2-morpholino-ethanamine. To hydrochloric acid (4 M, 143.25 mL) at 0° C. was added tert-butyl N-methyl-N-(2-morpholinoethyl) carbamate (14 g, 57.3 mmol) and the mixture stirred at rt for 50 min. The reaction mixture was concentrated and the residue was treated with NH₃ (7 M, 81.86 mL) and stirred for 1 h, then concentrated. The residue was purified via silica gel chromatography (DCM:MeOH:TEA=90:10:0.1) to provide the titled compound (7.4 g, 90% yield) as a yellow solid. ESI-MS (EI⁺, m/z): 145.1 [M+H]⁺. ¹H NMR (400 MHz, DMSO-d₆) δ 9.03 (s, 2H), 3.80 (s, 4H), 3.26 (dd, J=44.9, 20.4 Hz, 8H), 2.63 (s, 3H).

Step 4: [(43S,45R,47R)-4-[(2R)-2-[(30E,32E,34E,35E,39R,40S,41R,42R,44S,46S,48S,49S,50R,51R,61R)-61-hydroxy-48,51-dimethoxy-39,40,41,42,52,53-hexamethyl-54,55,56,57,58-pentaoxo-50-trimethylsilyloxy-73,74-dioxa-63-azatricyclohexatriaconta-30,32,34(52),35(53)-tetraen-49-yl]propyl]-47-methoxy-45-cyclohexyl] N-methyl-N-(2-morpholinoethyl)carbamate. To a solution of (25E,27E,29E,30E,32R,33S,34R,35R,37S,39S,41S,42S,43R,44R,53R)-53-hydroxy-42-[(1R)-2-[(1S,3R,4R)-4-hydroxy-3-methoxy-cyclohexyl]-1-methyl-ethyl]-41,44-dimethoxy-32,33,34,35,45,46-hexamethyl-43-trimethylsilyloxy-62,63-dioxa-54-azatricyclohexatriaconta-25,27,29(45),30(46)-tetraene-47,48,49,50,51-pentone (0.5 g, 0.507 mmol) and pyridine (160.4 mg, 2.03 mmol, 164 μL) in DCM (5 mL) at 0° C. under argon was added triphosgene (150.43 mg, 0507 mmol, in THF (20 mL)) dropwise via syringe. The reaction mixture was stirred for 1 h at 0° C. then triethylamine (0.41 g, 4.06 mmol) and N-methyl-2-morpholino-ethanamine (1.46 g, 10.14 mmol) were added and the resulting solution was stirred at 0° C. for another 1 h, diluted with DCM, washed with aqueous NH₄Cl solution, water, brine, dried over Na₂SO₄, filtered and concentrated. The residue was purified via silica gel chromatography (8% MeOH in DCM) to provide the titled compound (386 mg, 66% yield) as a light yellow solid. ESI-MS (EI⁺, m/z): 1156.4 [M+H]⁺. ¹H NMR (400 MHz, CDCl₃) δ 6.57-5.93 (m, 4H), 5.73-5.47 (m, 1H), 5.27-4.98 (m, 2H), 4.72 (s, 1H), 4.56 (s, 1H), 4.36-3.54 (m, 12H), 3.54-3.05 (m, 12H), 2.93 (s, 4H), 2.40 (dt, J=34.4, 23.8 Hz, 11H), 2.04 (s, 5H), 1.88-1.52 (m, 12H), 1.52-1.17 (m, 10H), 1.20-0.73 (m, 17H), 0.10-0.14 (m, 9H).

Step 5: [(40S,42R,44R)-4-[(2R)-2-[(27E,29E,31E,32E,36R,37S,38R,39R,41S,43S,45S,46S,47R,48R,58R)-47,58-dihydroxy-45,48-dimethoxy-36,37,38,39,49,50-hexamethyl-51,52,53,54,55-pentaoxo-71,72-dioxa-60-azatricyclohexatriaconta-27,29,31(49),32(50)-tetraen-46-yl]propyl]-44-methoxy-42-cyclohexyl] N-methyl-N-(2-morpholinoethyl)carbamate. To a solution of [(43S,45R,47R)-4-[(2R)-2-[(30E,32E,34E,35E,39R,40S,41R,42R,44S,46S,48S,49S,50R,51R,61R)-61-hydroxy-48,51-dimethoxy-39,40,41,42,52,53-hexamethyl-54,55,56,57,58-pentaoxo-50-trimethylsilyloxy-73,74-dioxa-63-azatricyclohexatriaconta-30,32,34(52),35(53)-tetraen-49-yl]propyl]-47-methoxy-45-cyclohexyl] N-methyl-N-(2-morpholinoethyl)carbamate (1.8 g, 1.56 mmol) in acetone (5 mL) and water (5 mL) was added 0.5 N sulfuric acid (4.67 mL) at 0° C. The resulting solution was stirred at 0° C. for 2 h, and then poured into a mixture of 100 mL EtOAc and 100 mL of saturated aqueous NaHCO₃ solution. The organic layer was washed with water and brine, dried over Na₂SO₄, filtered and concentrated. The residue was purified via silica gel chromatography (5% MeOH in DCM) to provide the titled compound (1.4 g, 83% yield) as a light yellow solid. ¹H NMR (400 MHz, CDCl₃) δ 6.47-5.84 (m, 4H), 5.60-5.05 (m, 4H), 4.77 (s, 1H), 4.55 (s, 1H), 4.34-4.10 (m, 1H), 3.92-3.52 (m, 7H), 3.52-3.23 (m, 10H), 3.13 (d, J=2.7 Hz, 4H), 2.92 (s, 3H), 2.78-2.39 (m, 8H), 2.40-2.00 (m, 5H), 2.03-1.53 (m, 18H), 1.53-1.11 (m, 12H), 1.11-0.87 (m, 13H), 0.83 (d, J=6.5 Hz, 2H).

Step 6: [(43S,45R,47R)-4-[(2R)-2-[(28E,30E,32E,33E,39R,40S,41R,42R,44S,46S,49S,51R,52R,62R)-51,62-dihydroxy-52-methoxy-39,40,41,42,53,54-hexamethyl-48-[2-(oxetan-3-yloxy)ethoxy]-55,56,57,58,59-pentaoxo-76,77-dioxa-64-azatricyclohexatriaconta-28,30,32(53),33(54)-tetraen-49-yl]propyl]-47-methoxy-45-cyclohexyl]N-methyl-N-(2-morpholinoethyl)carbamate (I-16). To a solution of [(40S,42R,44R)-4-[(2R)-2-[(27E,29E,31E,32E,36R,37S,38R,39R,41S,43S,45S,46S,47R,48R,58R)-47,58-dihydroxy-45,48-dimethoxy-36,37,38,39,49,50-hexamethyl-51,52,53,54,55-pentaoxo-71,72-dioxa-60-azatricyclohexatriaconta-27,29,31(49),32(50)-tetraen-46-yl]propyl]-44-methoxy-42-cyclohexyl] N-methyl-N-(2-morpholinoethyl)carbamate (0.2 g, 0.18 mmol) in DCM (5 mL) under nitrogen was added TFA (426 μL, 5.53 mmol) at −10° C. followed by 2-(oxetan-3-yloxy) ethanol (0.436 g, 3.69 mmol) and the mixture was stirred at −10° C. for 2 h then washed with aqueous NaHCO₃ solution, water and brine, filtered and concentrated. The residue was purified via reverse phase chromatography (65% CH₃CN in water) to provide I-16 (63 mg, 29% yield) as a white solid. ESI-MS (EI⁺, m/z): 1170.8 [M+H]⁺. ¹H NMR (400 MHz, CDCl₃) δ 6.46-6.01 (m, 4H), 5.56-5.15 (m, 4H), 4.75 (s, 2H), 4.60 (s, 3H), 4.18 (s, 2H), 3.72 (s, 6H), 3.64-3.03 (m, 13H), 2.94 (s, 3H), 2.80-2.28 (m, 9H), 2.13-1.87 (m, 4H), 1.84-1.38 (m, 24H), 1.85-0.75 (m, 25H).

Step 7: [(43S,45R,47R)-4-[(2R)-2-[(28E,30E,32E,33E,39R,40S,41R,42R,44S,46S,48S,49S,51R,52R,62R)-51,62-dihydroxy-52-methoxy-39,40,41,42,53,54-hexamethyl-48-[2-(oxetan-3-yloxy)ethoxy]-55,56,57,58,59-pentaoxo-76,77-dioxa-64-azatricyclohexatriaconta-28,30,32(53),33(54)-tetraen-49-yl]propyl]-47-methoxy-45-cyclohexyl] N-methyl-N-(2-morpholinoethyl)carbamate (I-18) and [(43S,45R,47R)-4-[(2R)-2-[(28E,30E,32E,33E,39R,40S,41R,42R,44S,46S,48R,49S,51R,52R,62R)-51,62-dihydroxy-52-methoxy-39,40,41,42,53,54-hexamethyl-48-[2-(oxetan-3-yloxy)ethoxy]-55,56,57,58,59-pentaoxo-76,77-dioxa-64-azatricyclohexatriaconta-28,30,32(53),33(54)-tetraen-49-yl]propyl]-47-methoxy-45-cyclohexyl] N-methyl-N-(2-morpholinoethyl)carbamate (I-17). 120 mg of I-16 was separated via chiral preparative HPLC to provide I-18 (27.5 mg, 23% yield) as a white solid and I-17 (16.1 mg, 13% yield) as a white solid.

Chiral analysis method:

Column: CHIRALPAK IC (IC00CE-UF123)

Column size: 0.46 cm I.D.×25 cm L

Injection: 20 μl

Mobile phase: EtOH=100%

Flow rate: 1.0 mL/min

Wave length: UV 254 nm

Temperature: 35° C.

I-18: ESI-MS (EI⁺, m/z): 1170.8 [M+H]⁺. ¹H NMR (400 MHz, CDCl₃) δ 6.40-5.78 (m, 4H), 5.62-5.03 (m, 4H), 4.76-4.39 (m, 6H), 4.12 (d, J=5.9 Hz, 1H), 3.88-3.59 (m, 6H), 3.56-3.00 (m, 17H), 2.86 (s, 3H), 2.79-2.18 (m, 11H), 2.15-1.81 (m, 5H), 1.59 (t, J=15.2 Hz, 13H), 1.49-1.15 (m, 11H), 1.10-0.66 (m, 18H).
I-17: ESI-MS (EI⁺, m/z): 1170.7 [M+H]⁺. ¹H NMR (400 MHz, CDCl₃) δ 6.49-5.81 (m, 4H), 5.64-5.06 (m, 4H), 4.66 (d, J=65.7 Hz, 3H), 4.23 (d, J=26.2 Hz, 2H), 3.94-3.03 (m, 28H), 2.98-2.22 (m, 15H), 2.21-1.69 (m, 11H), 1.54-1.18 (m, 13H), 1.15-0.69 (m, 19H).

### Example 12: [(42S,44R,46R)-4-[(2R)-2-[(27E,29E,31E,32E,38R,39S,40R,41R,43S,45S,48S,50R,51R,61R)-50,61-dihydroxy-51-methoxy-38,39,40,41,52,53-hexamethyl-47-[2-(oxetan-3-yloxy)ethoxy]-54,55,56,57,58-pentaoxo-75,76-dioxa-64-azatricyclohexatriaconta-27,29,31(52),32(53)-tetraen-48-yl]propyl]-46-methoxy-44-cyclohexyl] N-(2-morpholinoethyl)carbamate (I-19), [(42S,44R,46R)-4-[(2R)-2-[(27E,29E,31E,32E,38R,39S,40R,41R,43S,45S,47S,48S,50R,51R,61R)-50,61-dihydroxy-51-methoxy-38,39,40,41,52,53-hexamethyl-47-[2-(oxetan-3-yloxy)ethoxy]-54,55,56,57,58-pentaoxo-75,76-dioxa-64-azatricyclohexatriaconta-27,29,31(52),32(53)-tetraen-48-yl]propyl]-46-methoxy-44-cyclohexyl] N-(2-morpholinoethyl)carbamate (I-21) and [(42S,44R,46R)-4-[(2R)-2-[(27E,29E,31E,32E,38R,39S,40R,41R,43S,45S,47R,48S,50R,51R,61R)-50,61-dihydroxy-51-methoxy-38,39,40,41,52,53-hexamethyl-47-[2-(oxetan-3-yloxy)ethoxy]-54,55,56,57,58-pentaoxo-75,76-dioxa-64-azatricyclohexatriaconta-27,29,31(52),32(53)-tetraen-48-yl]propyl]-46-methoxy-44-cyclohexyl] N-(2-morpholinoethyl)carbamate (I-20)

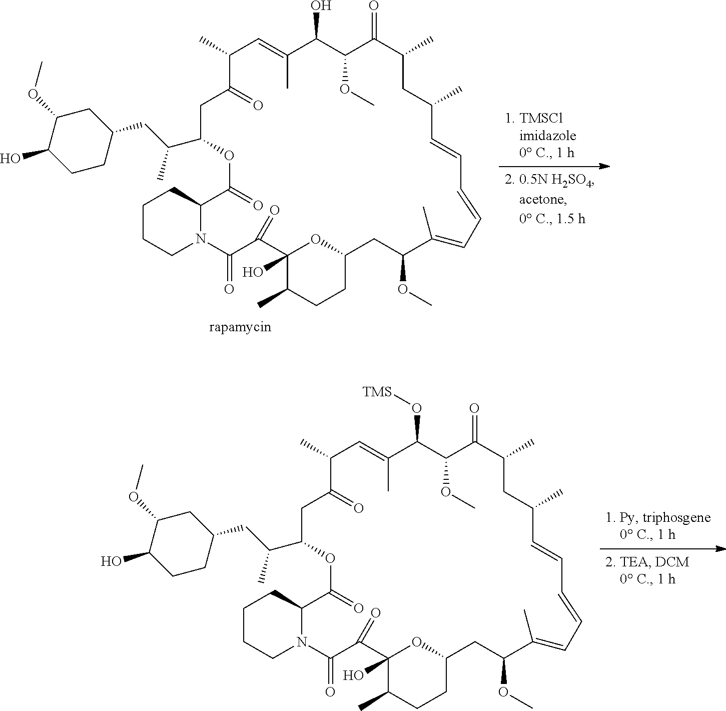

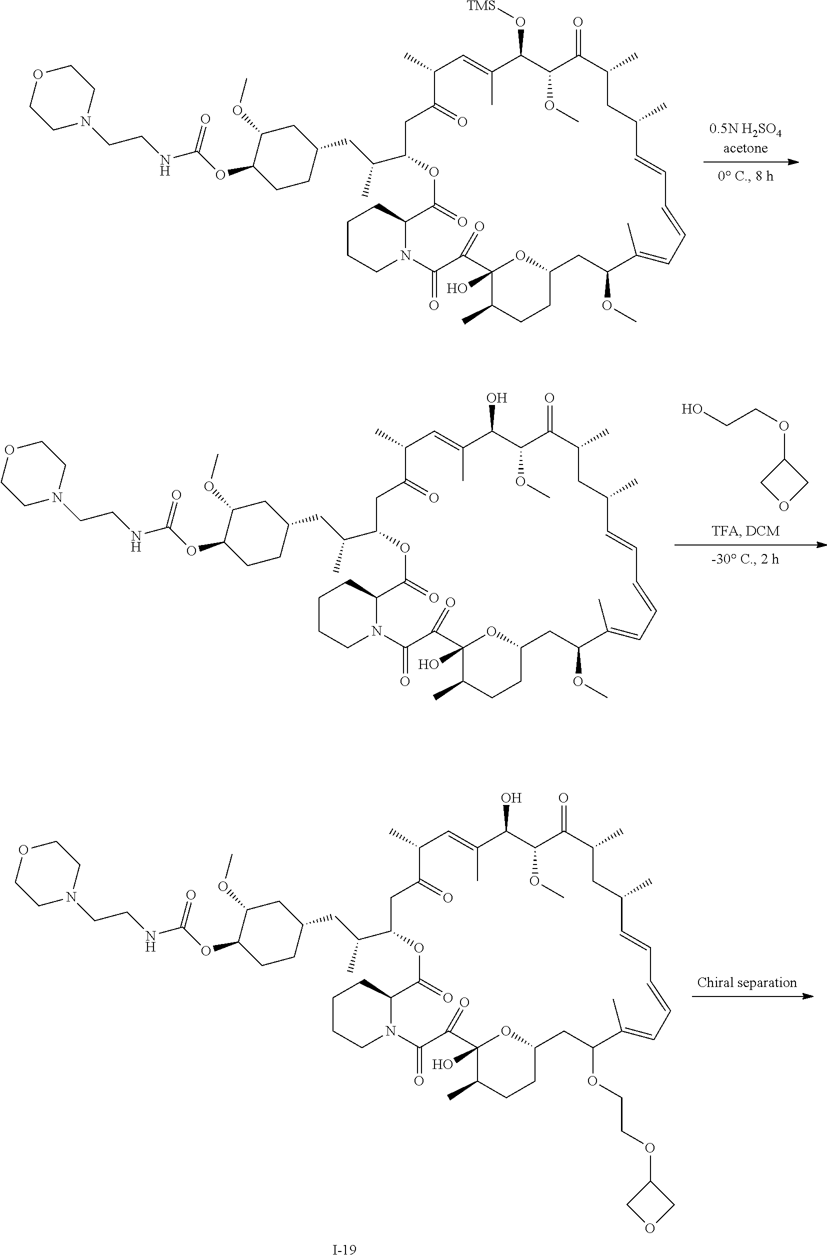

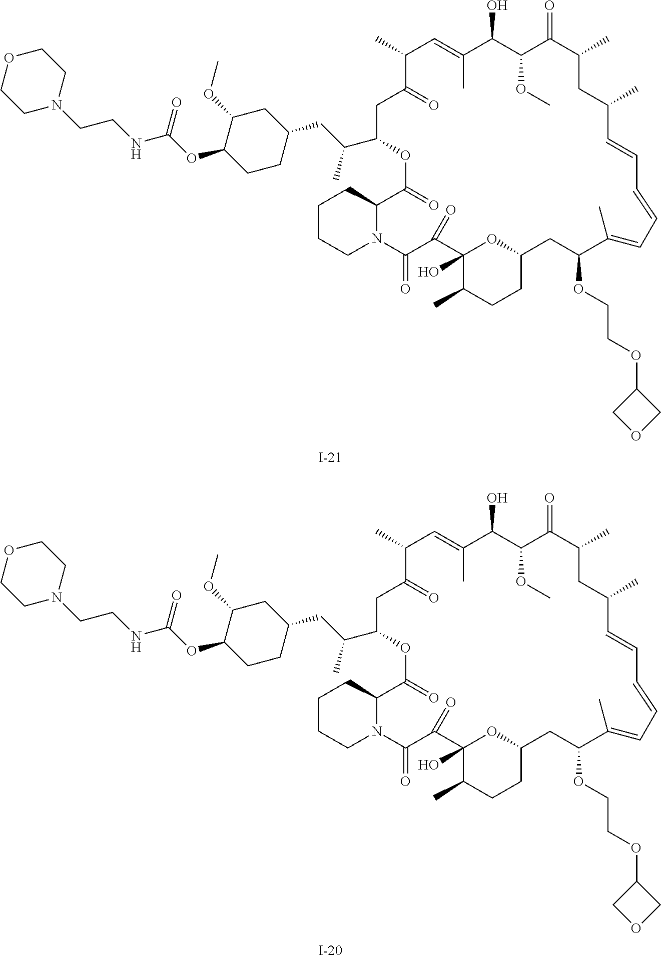

Step 1: (25E,27E,29E,30E,32R,33S,34R,35R,37S,39S,41S,42S,43R,44R,53R)-53-hydroxy-42-[(1R)-2-[(1S,3R,4R)-4-hydroxy-3-methoxy-cyclohexyl]-1-methyl-ethyl]-41,44-dimethoxy-32,33,34,35,45,46-hexamethyl-43-trimethylsilyloxy-62,63-dioxa-54-azatricyclohexatriaconta-25,27,29(45),30(46)-tetraene-47,48,49,50,51-pentone. To a solution of rapamycin (5.5 g, 6.02 mmol) and imidazole (3.2 g, 48 mmol) in EtOAc (35 mL) was added TMSCl (5.2 g, 48 mmol) dropwise at 0° C. After the addition, the resulting solution was stirred at rt for 1 h, then 0.5 N H₂SO₄ solution (24 mL) was added at 0° C. After the addition, the resulting solution was stirred at 0° C. for 1.5 h, then diluted with EtOAc (100 mL) and brine (50 mL), the organic layer was dried over anhydrous Na₂SO₄, filtered and concentrated. The residue was purified via silica gel chromatography to obtain the titled compound (4.33 g, 73% for 2 steps) as a white solid. ESI-MS (EI⁺, m/z): 1008.2 [M+Na]⁺. ¹H-NMR (400 MHz, CDCl₃) δ 6.50-5.80 (m, 4H), 5.61 (ddd, J=23.0, 14.1, 7.6 Hz, 1H), 5.37-5.19 (m, 2H), 5.07 (ddd, J=11.3, 9.1, 5.2 Hz, 1H), 4.71 (d, J=1.4 Hz, 1H), 3.89-3.56 (m, 4H), 3.50-3.30 (m, 6H), 3.29-3.18 (m, 3H), 3.18-3.04 (m, 3H), 2.97-2.86 (m, 1H), 2.84-2.45 (m, 3H), 2.43-2.05 (m, 4H), 1.97 (dd, J=10.0, 5.4 Hz, 2H), 1.86-1.50 (m, 19H), 1.49-0.81 (m, 23H), 0.68 (dd, J=23.6, 11.9 Hz, 1H), 0.05-0.07 (m, 9H).

Step 2: [(42S,44R,46R)-4-[(2R)-2-[(29E,31E,33E,34E,38R,39S,40R,41R,43S,45S,47S,48S,49R,50R,60R)-60-hydroxy-47,50-dimethoxy-38,39,40,41,51,52-hexamethyl-53,54,55,56,57-pentaoxo-49-trimethylsilyloxy-72,73-dioxa-63-azatricyclohexatriaconta-29,31,33(51),34(52)-tetraen-48-yl]propyl]-46-methoxy-44-cyclohexyl] N-(2-morpholinoethyl)carbamate. To a solution of (25E,27E,29E,30E,32R,33S,34R,35R,37S,39S,41S,42S,43R,44R,53R)-53-hydroxy-42-[(1R)-2-[(1S,3R,4R)-4-hydroxy-3-methoxy-cyclohexyl]-1-methyl-ethyl]-41,44-dimethoxy-32,33,34,35,45,46-hexamethyl-43-trimethylsilyloxy-62,63-dioxa-54-azatricyclohexatriaconta-25,27,29(45),30(46)-tetraene-47,48,49,50,51-pentone (2 g, 2.03 mmol) and pyridine (0.64 g, 8.11 mmol) in DCM (40 mL) was added triphosgene (0.6 g, 2.03 mmol in 10 mL DCM) dropwise by syringe at 0° C. under argon. The reaction mixture was stirred for 1 h at 0° C. then TEA (0.62 g, 6.08 mmol) and 2-morpholinoethanamine (0.53 g, 4.06 mmol) were added to the mixture and the resulting solution was stirred at 0° C. for another 1 h, then diluted with DCM, washed with aqueous NH₄Cl solution, water, brine, dried over Na₂SO₄, filtered and concentrated. The residue was purified via silica gel chromatography (8% MeOH in DCM) to provide the titled compound (2 g, 86% yield) as a light yellow solid.

Step 3: [(39S,41R,43R)-4-[(2R)-2-[(26E,28E,30E,31E,35R,36S,37R,38R,40S,42S,44S,45S,46R,47R,57R)-46,57-dihydroxy-44,47-dimethoxy-35,36,37,38,48,49-hexamethyl-50,51,52,53,54-pentaoxo-70,71-dioxa-60-azatricyclohexatriaconta-26,28,30(48),31(49)-tetraen-45-yl]propyl]-43-methoxy-41-cyclohexyl] N-(2-morpholinoethyl)carbamate. To a solution of [(42S,44R,46R)-4-[(2R)-2-[(29E,31E,33E,34E,38R,39S,40R,41R,43S,45S,47S,48S,49R,50R,60R)-60-hydroxy-47,50-dimethoxy-38,39,40,41,51,52-hexamethyl-53,54,55,56,57-pentaoxo-49-trimethylsilyloxy-72,73-dioxa-63-azatricyclohexatriaconta-29,31,33(51),34(52)-tetraen-48-yl]propyl]-46-methoxy-44-cyclohexyl]-N-(2-morpholinoethyl)carbamate (2 g, 1.75 mmol) in acetone (40 mL) and H₂O (10 mL) was added 0.5 N H₂SO₄ (2.63 mmol, 5.3 mL) at 0° C. The resulting solution was stirred at 0° C. for 8 h then poured into a mixture of 100 mL EtOAc and 100 mL of saturated aqueous NaHCO₃ solution. The organic layer was washed with water and brine, dried over Na₂SO₄, filtered and concentrated. The residue was purified via silica gel chromatography (5% MeOH in DCM) to provide the titled compound (1.5 g, 80% yield) as a light yellow solid.

Step 4: [(42S,44R,46R)-4-[(2R)-2-[(27E,29E,31E,32E,38R,39S,40R,41R,43S,45S,48S,50R,51R,61R)-50,61-dihydroxy-51-methoxy-38,39,40,41,52,53-hexamethyl-47-[2-(oxetan-3-yloxy)ethoxy]-54,55,56,57,58-pentaoxo-75,76-dioxa-64-azatricyclohexatriaconta-27,29,31(52),32(53)-tetraen-48-yl]propyl]-46-methoxy-44-cyclohexyl]N-(2-morpholinoethyl)carbamate. To a solution of [(39S,41R,43R)-4-[(2R)-2-[(26E,28E,30E,31E,35R,36S,37R,38R,40S,42S,44S,45S,46R,47R,57R)-46,57-dihydroxy-44,47-dimethoxy-35,36,37,38,48,49-hexamethyl-50,51,52,53,54-pentaoxo-70,71-dioxa-60-azatricyclohexatriaconta-26,28,30(48),31(49)-tetraen-45-yl]propyl]-43-methoxy-41-cyclohexyl] N-(2-morpholinoethyl)carbamate (02 g, 0.18 mmol) in DCM (4 mL) under nitrogen was added TFA (0.85 g, 7.47 mmol) at −40° C. 2-(oxetan-3-yloxy) ethanol (0.22 g, 1.87 mmol) was added and the mixture was stirred at −30° C. for 2 h. The mixture was poured into cold saturated aqueous NaHCO₃ (30 mL), extracted with DCM (30 mL), washed with water (30 mL) and brine (30 mL), dried over anhydrous Na₂SO₄, filtered and concentrated. The residue was purified by reverse phase column eluting with 80% CH₃CN in water to provide I-19 (35 mg, 16% yield) as a white solid. ESI-MS (EI⁺, m/z): 1179.6 [M+Na]⁺. ¹H NMR (400 MHz, CDCl₃) δ 6.41-5.90 (m, 4H), 5.58-5.39 (m, 2H), 5.30-5.15 (m, 2H), 4.80-4.51 (m, 5H), 4.32-3.95 (m, 2H), 3.92-3.66 (m, 7H), 3.60-3.40 (m, 4H), 3.39-3.20 (m, 11H), 3.19-3.05 (m, 2H), 2.79-2.62 (m, 2H), 2.61-2.40 (m, 7H), 2.37-2.20 (m, 2H), 2.15-1.90 (m, 5H), 1.84-1.58 (m, 17H), 1.54-1.16 (m, 7H), 1.15-0.83 (m, 17H), 0.82-0.75 (m, 1H).

Step 5: [(42S,44R,46R)-4-[(2R)-2-[(27E,29E,31E,32E,38R,39S,40R,41R,43S,45S,47S,48S,50R,51R,61R)-50,61-dihydroxy-51-methoxy-38,39,40,41,52,53-hexamethyl-47-[2-(oxetan-3-yloxy)ethoxy]-54,55,56,57,58-pentaoxo-75,76-dioxa-64-azatricyclohexatriaconta-27,29,31(52),32(53)-tetraen-48-yl]propyl]-46-methoxy-44-cyclohexyl] N-(2-morpholinoethyl)carbamate (I-21) and [(42S,44R,46R)-4-[(2R)-2-[(27E,29E,31E,32E,38R,39S,40R,41R,43S,45S,47R,48S,50R,51R,61R)-50,61-dihydroxy-51-methoxy-38,39,40,41,52,53-hexamethyl-47-[2-(oxetan-3-yloxy)ethoxy]-54,55,56,57,58-pentaoxo-75,76-dioxa-64-azatricyclohexatriaconta-27,29,31(52),32(53)-tetraen-48-yl]propyl]-46-methoxy-44-cyclohexyl] N-(2-morpholinoethyl)carbamate (I-20)

115 mg of [(42S,44R,46R)-4-[(2R)-2-[(27E,29E,31E,32E,38R,39S,40R,41R,43S,45S,48S,50R,51R,61R)-50,61-dihydroxy-51-methoxy-38,39,40,41,52,53-hexamethyl-47-[2-(oxetan-3-yloxy)ethoxy]-54,55,56,57,58-pentaoxo-75,76-dioxa-64-azatricyclohexatriaconta-27,29,31(52),32(53)-tetraen-48-yl]propyl]-46-methoxy-44-cyclohexyl]N-(2-morpholinoethyl)carbamate was separated via chiral preparative HPLC then purified via silica gel chromatography (12% MeOH in petroleum ether:DCM:EA=3:3:1) to provide I-21 (23 mg, 20% yield) as a white solid and I-20 (10 mg, 8.7% yield) as a white solid.

Chiral separation method:

Column: CHIRALPAK IC

Column size: 2.5 cm I.D.×25 cm L, 10 μm

Sample solution: 3 mg/mL in mobile phase

Injection: 10 mL

Mobile phase: EtOH=100%

Flow rate: 10 mL/min

Wave length: UV 254 nm

Temperature: 35° C.

I-21: ESI-MS (EI⁺, m/z): 1156.9 [M+H]⁺, 1178.8 [M+Na]⁺. ¹H NMR (400 MHz, CDCl₃) δ 6.51-5.78 (m, 4H), 5.74-5.01 (m, 5H), 4.68 (ddd, J=37.1, 11.1, 6.1 Hz, 6H), 4.32-4.13 (m, 1H), 3.96-3.06 (m, 23H), 2.79-2.24 (m, 10H), 2.17-1.18 (m, 29H), 1.17-0.76 (m, 19H).
I-20: ESI-MS (EI⁺, m/z): 1156.9 [M+H]⁺, 1178.8 [M+Na]⁺. ¹H NMR (400 MHz, CDCl₃) δ 6.58-5.81 (m, 4H), 5.67-5.02 (m, 5H), 4.44 (dd, J=176.8, 44.7 Hz, 6H), 4.03-3.05 (m, 26H), 2.83-2.29 (m, 10H), 2.17-1.19 (m, 27H), 1.16-0.69 (m, 19H).

### Example 13: (22E,24E,26E,27E,35R,36S,37R,38R,40S,42S,45S,46R,47R,56R)-44-[2-(2-aminoethoxy)ethoxy]-56-hydroxy-45-[(1R)-2-[(1S,3R,4R)-4-(2-hydroxyethoxy)-3-methoxy-cyclohexyl]-1-methyl-ethyl]-46,47-dimethoxy-35,36,37,38,48,49-hexamethyl-66,67-dioxa-58-azatricyclohexatriaconta-22,24,26(48),27(49)-tetraene-50,51,52,53,54-pentone (I-23)

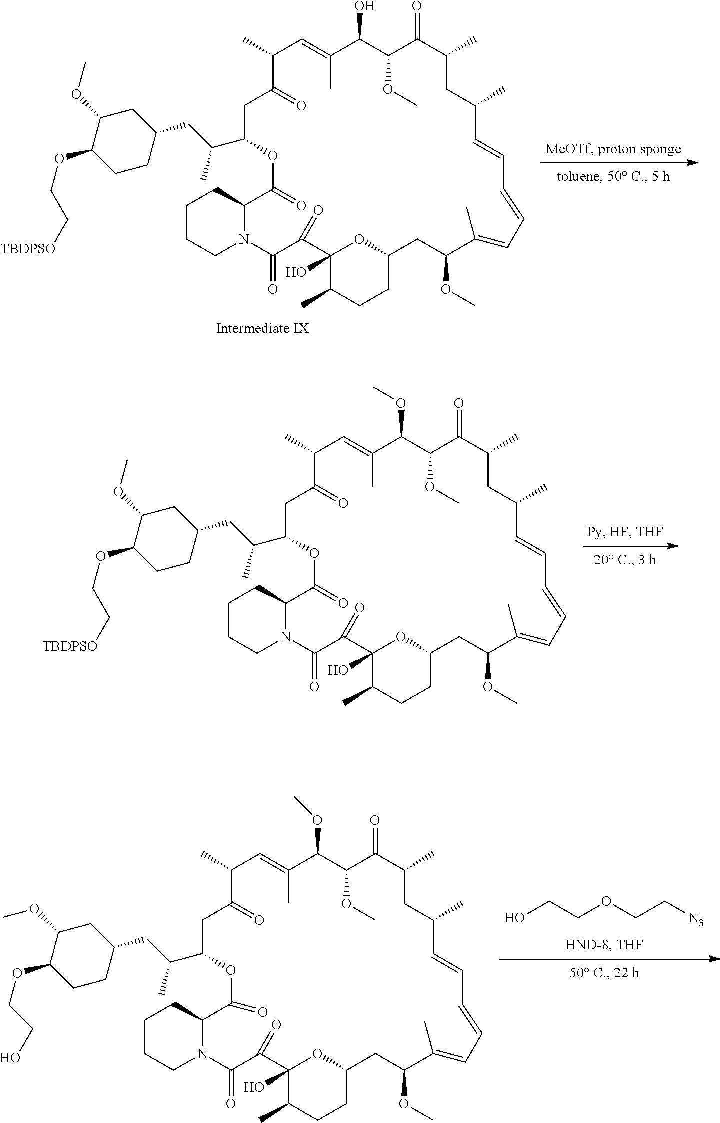

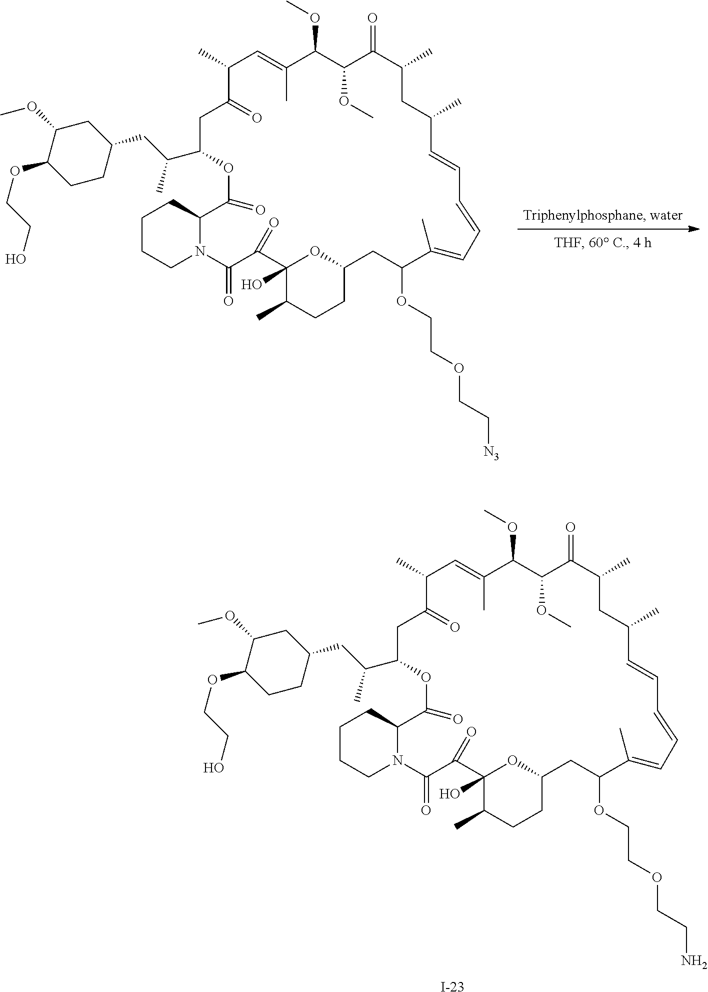

Step 1: (36E,38E,40E,41E,47R,48S,49R,50R,52S,54S,56S,57S,58R,59R,68R)-57-[(1R)-2-[(1S,3R,4R)-4-[2-[tert-butyl(diphenyl)silyl]oxyethoxy]-3-methoxy-cyclohexyl]-1-methyl-ethyl]-68-hydroxy-56,58,59-trimethoxy-47,48,49,50,60,61-hexamethyl-77,78-dioxa-70-azatricyclohexatriaconta-36,38,40(60),41(61)-tetraene-62,63,64,65,66-pentone. To a solution of (35E,37E,39E,40E,46R,47S,48R,49R,51S,53S,55S,56S,57R,58R,67R)-56-[(1R)-2-[(1S,3R,4R)-4-[2-[tert-butyl(diphenyl)silyl]oxyethoxy]-3-methoxy-cyclohexyl]-1-methyl-ethyl]-57,67-dihydroxy-55,58-dimethoxy-46,47,48,49,59,60-hexamethyl-77,78-dioxa-69-azatricyclohexatriaconta-35,37,39(59),40(60)-tetraene-61,62,63,64,65-pentone (Intermediate IX is prepared according to Example 22, 2 g, 1.67 mmol) and 1,8-bis(dimethylamino)naphtalene (3.94 g, 18.39 mmol) in toluene (40 mL) was added methyl trifluoromethanesulfonate (2.19 g, 13.37 mmol) dropwise at room temperature under N₂. After addition, the mixture was heated to 50° C. for 5 h then cooled, filtered and diluted with EA (60 mL), washed with sat. NH₄Cl (aq) (60 mL×3), water (60 mL) and brine (60 mL). The organic layer was dried over anhydrous sodium sulfate, filtered and concentrated. The residue was purified via silica gel chromatography (petroleum ether:EA=3:1) to provide the titled compound (700 mg, 35% yield) as a yellow solid. ESI-MS (EI⁺, m/z): 1232.7 [M+Na]⁺.

Step 2: (23E,25E,27E,28E,32R,33S,34R,35R,37S,39S,41S,42S,43R,44R,53R)-53-hydroxy-42-[(1R)-2-[(1S,3R,4R)-4-(2-hydroxyethoxy)-3-methoxy-cyclohexyl]-1-methyl-ethyl]-41,43,44-trimethoxy-32,33,34,35,45,46-hexamethyl-62,63-dioxa-54-azatricyclohexatriaconta-23,25,27(45),28(46)-tetraene-47,48,49,50,51-pentone. To a solution of (36E,38E,40E,41E,47R,48S,49R,50R,52S,54S,56S,57S,58R,59R,68R)-57-[(1R)-2-[(1S,3R,4R)-4-[2-[tert-butyl(diphenyl)silyl]oxyethoxy]-3-methoxy-cyclohexyl]-1-methyl-ethyl]-68-hydroxy-56,58,59-trimethoxy-47,48,49,50,60,61-hexamethyl-77,78-dioxa-70-azatricyclohexatriaconta-36,38,40(60),41(61)-tetraene-62,63,64,65,66-pentone (600 mg, 0.496 mmol) in THF (10 mL) was added pyridine HF (392 mg, 4.96 mmol) at 0° C. The mixture was stirred at 30° C. for 3 h then quenched by adding saturated aqueous NaHCO₃ (20 mL) and extracted with EA (30 mL) at 0° C. The organic layer was washed with water (20 mL) and brine (20 mL), dried over anhydrous sodium sulfate, filtered and concentrated. The residue was purified via silica gel chromatography (petroleum ether:acetone=3:1) to provide the titled compound (430 mg, 89% yield) as a light yellow solid. ESI-MS (EI⁺, m/z): 994.7 [M+Na]⁺.

Step 3: (22E,24E,26E,27E,35R,36S,37R,38R,40S,42S,45S,46R,47R,56R)-44-[2-(2-azidoethoxy)ethoxy]-56-hydroxy-45-[(1R)-2-[(1S,3R,4R)-4-(2-hydroxyethoxy)-3-methoxy-cyclohexyl]-1-methyl-ethyl]-46,47-dimethoxy-35,36,37,38,48,49-hexamethyl-68,69-dioxa-60-azatricyclohexatriaconta-22,24,26(48),27(49)-tetraene-50,51,52,53,54-pentone. To a solution of (23E,25E,27E,28E,32R,33S,34R,35R,37S,39S,41S,42S,43R,44R,53R)-53-hydroxy-42-[(1R)-2-[(1S,3R,4R)-4-(2-hydroxyethoxy)-3-methoxy-cyclohexyl]-1-methyl-ethyl]-41,43,44-trimethoxy-32,33,34,35,45,46-hexamethyl-62,63-dioxa-54-azatricyclohexatriaconta-23,25,27(45),28(46)-tetraene-47,48,49,50,51-pentone (450 mg, 462.85 μmol) and 2-(2-azidoethoxy)ethanol (1.21 g, 9.26 mmol) in THF (10 mL) was added HND-8 (100 mg) at 50° C. under N₂. The reaction mixture was stirred for 22 h at 50° C. then cooled and filtered. The filtrate was poured into a solution of saturated aqueous NaHCO₃ (10 mL) at 0° C. and extracted with EA (30 mL), the organic layer was washed with water (10 mL) and brine (10 mL), dried over anhydrous sodium sulfate, filtered and concentrated. The residue was purified by reverse-phase chromatography (70% CH₃CN in water) to provide the titled compound (250 mg, 50% yield) as a light yellow solid. ESI-MS (EI⁺, m/z): 1093.4 [M+Na]⁺.

Step 4: (22E,24E,26E,27E,35R,36S,37R,38R,40S,42S,45S,46R,47R,56R)-44-[2-(2-aminoethoxy)ethoxy]-56-hydroxy-45-[(1R)-2-[(1S,3R,4R)-4-(2-hydroxyethoxy)-3-methoxy-cyclohexyl]-1-methyl-ethyl]-46,47-dimethoxy-35,36,37,38,48,49-hexamethyl-66,67-dioxa-58-azatricyclohexatriaconta-22,24,26(48),27(49)-tetraene-50,51,52,53,54-pentone (I-23). To a solution of (22E,24E,26E,27E,35R,36S,37R,38R,40S,42S,45S,46R,47R,56R)-44-[2-(2-azidoethoxy)ethoxy]-56-hydroxy-45-[(1R)-2-[(1S,3R,4R)-4-(2-hydroxyethoxy)-3-methoxy-cyclohexyl]-1-methyl-ethyl]-46,47-dimethoxy-35,36,37,38,48,49-hexamethyl-68,69-dioxa-60-azatricyclohexatriaconta-22,24,26(48),27(49)-tetraene-50,51,52,53,54-pentone (0.6 g, 0.56 mmol) in THF (10 mL) was added triphenylphosphine (0.44 g, 1.68 mmol) slowly. The resulting solution was stirred at 60° C. for 2 h, 0.05 ml of water was added, the reaction stirred at room temperature for 6 h then concentrated. The residue was purified by reverse-phase chromatography (CH₃CN/H₂O with 0.025% TFA) to provide I-23 (40 mg, 7% yield) as a white solid. ESI-MS (EI⁺, m/z): 1045.7 [M+H]⁺. ¹H NMR (400 MHz, CDCl₃) δ 4.61-5.96 (m, 4H), 5.69-5.07 (m, 4H), 4.51-4.01 (m, 3H), 3.82-3.52 (m, 7H), 3.47-3.37 (m, 5H), 3.31-3.04 (m, 13H), 2.88-2.52 (m, 2H), 2.38-1.97 (m, 7H), 1.85-1.52 (m, 17H), 1.38-1.13 (m, 7H), 1.12-0.98 (m, 5H), 0.98-0.77 (m, 17H), 0.75-0.68 (m, 1H).

### Example 14: Synthesis of (1R,2R,4S)-4-((2R)-2-((3S,6R,7E,9R,10R,12R,14S,15E,17E,19E,23S,26R,27R,34aS)-21-((1,4-dioxan-2-yl)methoxy)-27-hydroxy-9,10-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)-2-methoxycyclohexyl dimethylphosphinate (I-24) and (27E,29E,31E,32E,35R,36S,37R,38R,40S,42S,44S,45S,47R,48R,57R)-45-[(1R)-2-[(1S,3R,4R)-4-dimethylphosphoryloxy-3-methoxy-cyclohexyl]-1-methyl-ethyl]-44-(1,4-dioxan-2-ylmethoxy)-57-hydroxy-47,48-dimethoxy-35,36,37,38,49,50-hexamethyl-68,69-dioxa-58-azatricyclohexatriaconta-27,29,31(49),32(50)-tetraene-51,52,53,54,55-pentone (I-25)

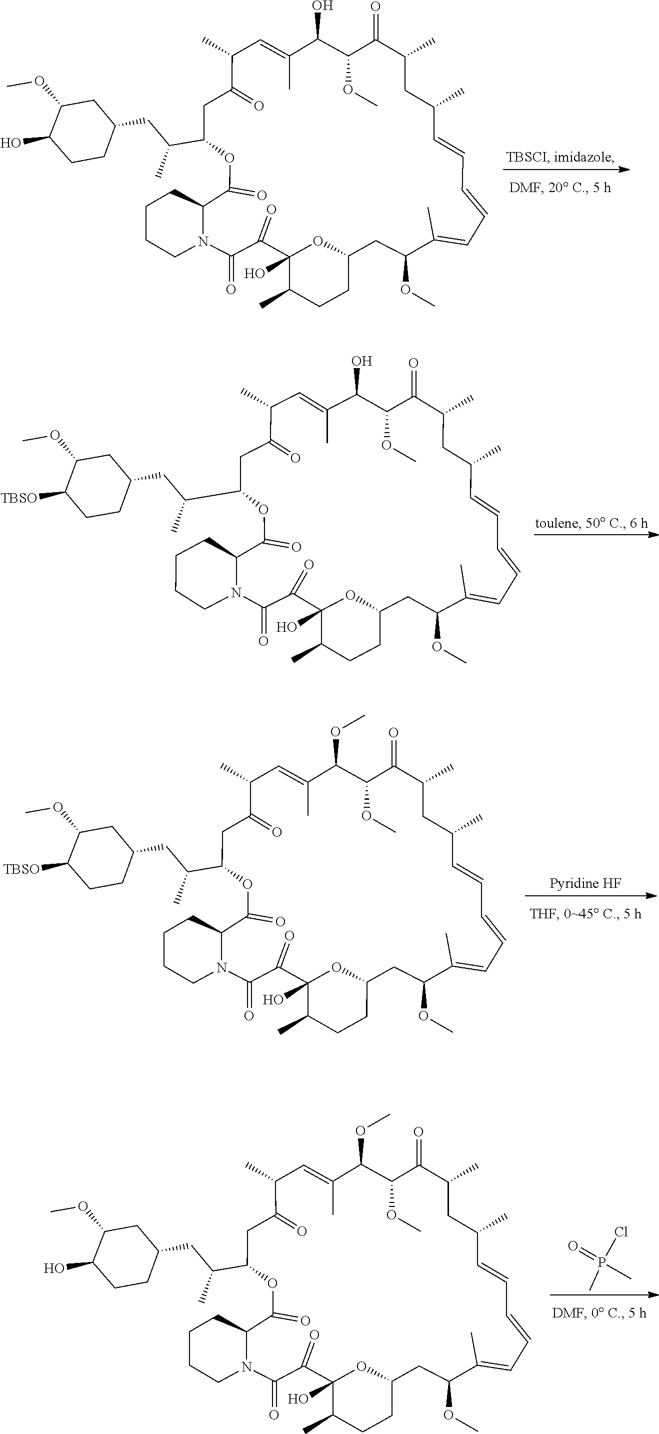

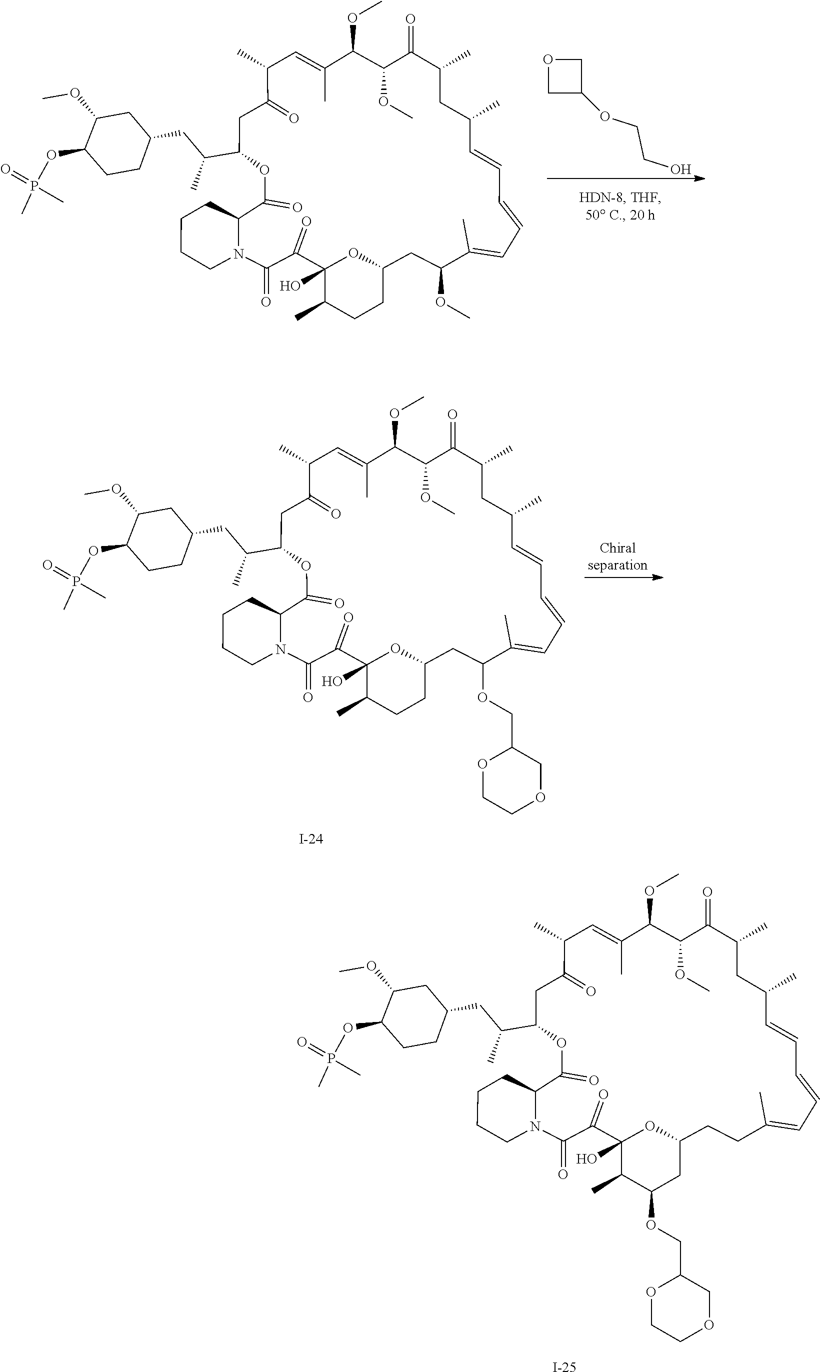

Step 1: (3S,6R,7E,9R,10R,12R,14S,15E,17E,19E,21S,23S,26R,27R,34aS)-3-((R)-1-((1S,3R,4R)-4-((tert-butyldimethylsilyl)oxy)-3-methoxycyclohexyl)propan-2-yl)-9,27-dihydroxy-10,21-dimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone. To a solution of rapamycin (5 g, 5.47 mmol) in DMF (60 mL) at rt was added imidazole (1.49 g, 21.88 mmol) and tert-butyl-chloro-dimethyl-silane (2.47 g, 16.41 mmol). The reaction was stirred at 50° C. for 6 h then poured into a mixture of ice cold saturated aqueous NH₄Cl (40 mL) and Et₂O:petroleum ether (60 mL, 2:1). The organic layer was washed with saturated aqueous NH₄Cl (20 mL), washed with water and brine (20 mL), dried over Na₂SO₄, filtered and concentrated. The residue was purified via silica gel chromatography (EtOAc in petroleum ether from 10% to 50%) to provide the titled compound (4 g, 71% yield) as a white solid. ESI-MS (EI⁺, m/z): 1050.5 [M+Na]⁺.

Step 2: (3S,6R,7E,9R,10R,12R,14S,15E,17E,19E,21S,23S,26R,27R,34aS)-3-((R)-1-((1S,3R,4R)-4-((tert-butyldimethylsilyl)oxy)-3-methoxycyclohexyl)propan-2-yl)-27-hydroxy-9,10,21-trimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone. To a suspension of (3S,6R,7E,9R,10R,12R,14S,15E,17E,19E,21S,23S,26R,27R,34aS)-3-((R)-1-((1S,3R,4R)-4-((tert- butyldimethylsilyl)oxy)-3-methoxycyclohexyl)propan-2-yl)-9,27-dihydroxy-10,21-dimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (1 g, 0.97 mmol) and 1,8-bis(dimethylamino)naphtalene (2.5 g, 11.67 mmol) in toluene (15 mL) was added methyl trifluoromethanesulfonate (1.6 mL, 14.59 mmol) dropwise at rt under N₂. After addition, the mixture was heated to 50° C. for 6 h. then cooled, filtered and the filtrate purified via silica gel chromatography to provide the titled compound (0.45 g, 0.43 mmol) as a white solid. ESI-MS (EI+, m/z): 1064.6 [M+Na]⁺.

Step 3: (3S,6R,7E,9R,10R,12R,14S,15E,17E,19E,21S,23S,26R,27R,34aS)-27-hydroxy-3-((R)-1-((1S,3R,4R)-4-hydroxy-3-methoxycyclohexyl)propan-2-yl)-9,10,21-trimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone. To a solution of (3S,6R,7E,9R,10R,12R,14S,15E,17E,19E,21S,23S,26R,27R,34aS)-3-((R)-1-((1S,3R,4R)-4-((tert-butyldimethylsilyl)oxy)-3-methoxycyclohexyl)propan-2-yl)-27-hydroxy-9,10,21-trimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (400 mg, 038 mmol) in THF (10 mL) was added pyridine hydrofluoride (3.34 mL, 38.37 mmol) at 0° C. The reaction was warmed to 45° C., stirred for 5 h then diluted with DCM and aqueous NaHCO₃ solution, washed with water and brine, dried over Na₂SO₄, filtered and concentrated. The residue was purified via silica gel chromatography to provide the titled compound (0.16 g, 45% yield) as a white solid. ESI-MS (EI⁺, m/z): 949.9 [M+Na]⁺.

Step 4: (1R,2R,4S)-4-((R)-2-((3S,6R,7E,9R,10R,12R,14S,15E,17E,19E,21S,23S,26R,27R,34aS)-27-hydroxy-9,10,21-trimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)-2-methoxycyclohexyl dimethylphosphinate. To a solution of (3S,6R,7E,9R,10R,12R,14S,15E,17E,19E,21S,23S,26R,27R,34aS)-27-hydroxy-3-((R)-1-((1S,3R,4R)-4-hydroxy-3-methoxycyclohexyl)propan-2-yl)-9,10,21-trimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (0.26 g, 0.28 mmol) in DCM (10 mL) was added 2,6-di-tert-butyl-4-methylpyridine (0.173 g, 0.84 mmol) and dimethylphosphinic chloride (0.315 g, 2.8 mmol) in DCM (1 mL) at 0° C. The resulting solution was stirred at 0° C. for 5 h then diluted with EtOAc, washed with aqueous NaHCO3 solution, ice cold 0.5 N HCl solution, water, brine, dried over Na₂SO₄, filtered and concentrated. The residue was purified via silica gel chromatography (DCM:MeOH=40:1) to provide the titled compound (0.1 g, 36% yield) as a white solid. ESI-MS (EI⁺, m/z): 1025.8 [M+Na]⁺.

Step 5: (1R,2R,4S)-4-((2R)-2-((3S,6R,7E,9R,10R,12R,14S,15E,17E,19E,23S,26R,27R,34aS)-21-((1,4-dioxan-2-yl)methoxy)-27-hydroxy-9,10-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)-2-methoxycyclohexyl dimethylphosphinate (I-24). To a solution of (1R,2R,4S)-4-((R)-2-((3S,6R,7E,9R,10R,12R,14S,15E,17E,19E,21S,23S,26R,27R,34aS)-27-hydroxy-9,10,21-trimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)-2-methoxycyclohexyl dimethylphosphinate (129 mg, 0.129 mmol) in DCM (5 mL) was added TFA (0.49 mL, 6.42 mmol) at −50° C. The mixture was stirred at the same temperature for 10 minutes then 2-(oxetan-3-yloxy) ethanol (0.758 g, 6.42 mmol) dissolved in DCM (0.5 mL) was added and the mixture stirred at 0° C. for 6 h. The reaction was then diluted with DCM and aqueous NaHCO₃ solution, washed with water and brine, dried over Na₂SO₄, filtered and concentrated. The residue was then purified via reverse-phase chromatography (70% CH₃CN in water) to provide I-24 (30 mg, 21% yield) as a white solid. ESI-MS (EI⁺, m/z): 1112.8 [M+Na]⁺. ¹H NMR (400 MHz, CDCl₃) δ 6.49 (br, 4H), 5.63-5.06 (m, 4H), 4.78-4.05 (m, 3H), 3.66 (ddd, J=23.6, 18.4, 8.5 Hz, 9H), 3.49-2.97 (m, 17H), 2.82-2.48 (m, 2H), 2.37-1.86 (m, 7H), 1.56-1.23 (m, 22H), 1.18-0.68 (m, 24H).

Step 6: (27E,29E,31E,32E,35R,36S,37R,38R,40S,42S,44S,45S,47R,48R,57R)-45-[(1R)-2-[(1S,3R,4R)-4-dimethylphosphoryloxy-3-methoxy-cyclohexyl]-1-methyl-ethyl]-44-(1,4-dioxan-2-ylmethoxy)-57-hydroxy-47,48-dimethoxy-35,36,37,38,49,50-hexamethyl-68,69-dioxa-58-azatricyclohexatriaconta-27,29,31(49),32(50)-tetraene-51,52,53,54,55-pentone (I-25). 100 mg of I-24 was separated via chiral preparative HPLC and then purified via silica gel chromatography (8% MeOH in petroleum ether:DCM:EA: 3:3:1) to provide I-25 (14 mg, 14% yield) as a white solid.

Chiral separation method:

Column: CHIRALPAK IC

Column size: 2.5 cm I.D.×25 cm L, 10 μm

Sample solution: 9 mg/mL in Mobile phase

Injection: 15 mL

Mobile phase: Hexane/EtOH=50/50(V/V)

Flow rate: 30 mL/min

Wave length: UV 254 nm

Temperature: 35° C.

ESI-MS (EI⁺, m/z): 1112.6 [M+Na]⁺. ¹H NMR (400 MHz, CDCl₃) δ 6.49-5.80 (m, 4H), 5.67-5.15 (m, 4H), 4.20-4.02 (m, 2H), 3.98-3.55 (m, 12H), 3.52-3.00 (m, 17H), 2.60 (ddd, J=39.8, 34.1, 28.4 Hz, 6H), 2.37-1.83 (m, 7H), 1.66 (dt, J=39.0, 20.7 Hz, 12H), 1.42-1.19 (m, 8H), 1.18-0.66 (m, 20H).

### Example 15: Synthesis of (25E,27E,29E,30E,33R,34S,35R,36R,38S,40S,43S,45R,46R,55R)-42-(1,4-dioxan-2-ylmethoxy)-55-hydroxy-43-[(1R)-2-[(1S,3R,4R)-4-hydroxy-3-methoxy-cyclohexyl]-1-methyl-ethyl]-45,46-dimethoxy-33,34,35,36,47,48-hexamethyl-66,67-dioxa-56-azatricyclohexatriaconta-25,27,29(47),30(48)-tetraene-49,50,51,52,53-pentone (I-26) and (25E,27E,29E,30E,33R,34S,35R,36R,38S,40S,42S,43S,45R,46R,55R)-42-(1,4-dioxan-2-ylmethoxy)-55-hydroxy-43-[(1R)-2-[(1S,3R,4R)-4-hydroxy-3-methoxy-cyclohexyl]-1-methyl-ethyl]-45,46-dimethoxy-33,34,35,36,47,48-hexamethyl-66,67-dioxa-56-azatricyclohexatriaconta-25,27,29(47),30(48)-tetraene-49,50,51,52,53-pentone (I-27)

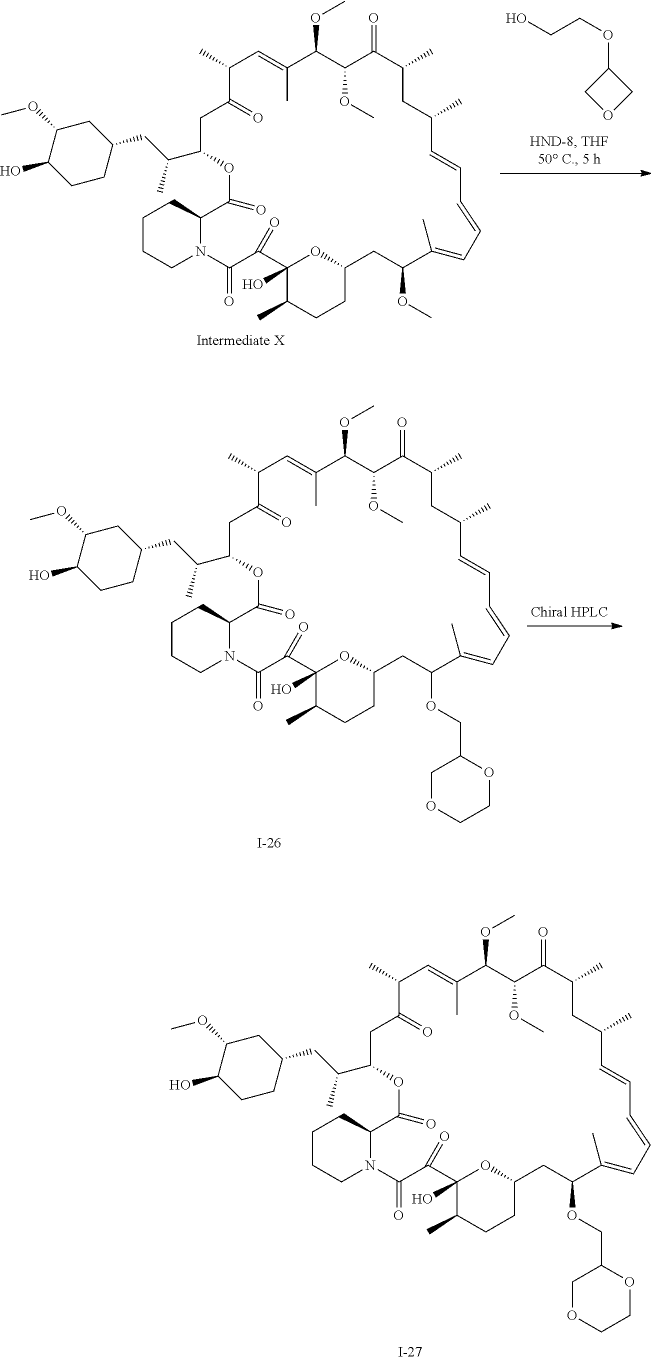

Step 1: (25E,27E,29E,30E,33R,34S,35R,36R,38S,40S,43S,45R,46R,55R)-42-(1,4-dioxan-2-ylmethoxy)-55-hydroxy-43-[(1R)-2-[(1S,3R,4R)-4-hydroxy-3-methoxy-cyclohexyl]-1-methyl-ethyl]-45,46-dimethoxy-33,34,35,36,47,48-hexamethyl-66,67-dioxa-56-azatricyclohexatriaconta-25,27,29(47),30(48)-tetraene-49,50,51,52,53-pentone (I-26). To a solution of (23E,25E,27E,28E,30R,31S,32R,33R,35S,37S,39S,40S,41R,42R,51R)-51-hydroxy-40-[(1R)-2-[(1S,3R,4R)-4-hydroxy-3-methoxy-cyclohexyl]-1-methyl-ethyl]-39,41,42-trimethoxy-30,31,32,33,43,44-hexamethyl-60,61-dioxa-52-azatricyclohexatriaconta-23,25,27(43),28(44)-tetraene-45,46,47,48,49-pentone (Intermediate X was prepared according to Example 16, 0.312 g, 0.336 mmol) in THF (15 mL) under nitrogen at 0° C. was added 2-(oxetan-3-yloxy)ethanol (0.397 g, 3.36 mmol) and HND-8 (624 mg). The mixture was stirred at 50° C. for 5 h then purified via reverse phase chromatography, eluting with 80% CH₃CN in water, and by TLC (petroleum ether:EtOAc=1:2) to provide I-26 (30 mg, 9% yield) as a white solid. ESI-MS (EI⁺, m/z): 1035.8 [M+Na]⁺. ¹H NMR (500 MHz, CDCl₃): ¹H NMR (500 MHz, CDCl₃) δ 6.57-5.90 (m, 3H), 5.71-5.00 (m, 3H), 4.72-4.10 (m, 1H), 3.91-3.52 (m, 7H), 3.38 (dd, J=22.8, 12.9 Hz, 5H), 3.30-3.15 (m, 3H), 3.16-3.02 (m, 3H), 3.00-2.46 (m, 4H), 2.15 (dd, J=97.2, 37.0 Hz, 5H), 1.85-1.53 (m, 23H), 1.52-1.21 (m, 9H), 1.19-0.82 (m, 14H), 0.69 (d, J=11.9 Hz, 1H).

Step 2: (25E,27E,29E,30E,33R,34S,35R,36R,38S,40S,42S,43S,45R,46R,55R)-42-(1,4-dioxan-2-ylmethoxy)-55-hydroxy-43-[(1R)-2-[(1S,3R,4R)-4-hydroxy-3-methoxy-cyclohexyl]-1-methyl-ethyl]-45,46-dimethoxy-33,34,35,36,47,48-hexamethyl-66,67-dioxa-56-azatricyclohexatriaconta-25,27,29(47),30(48)-tetraene-49,50,51,52,53-pentone (I-27). 85 mg of the epimeric mixture was purified via preparative chiral HPLC and then by silica gel chromatography (hexane:DCM:EtOAc:MeOH=3:3:1:0.3) to provide I-27 (25 mg, 29% yield) as a white solid.

Chiral separation method:

Column: CHIRALPAK IC

Column size: 5.0 cm I.D.×25 cm L, 10 μm

Sample solution: 0.3 mg/mL in Mobile phase

Injection: 3 mL

Mobile phase: Hexane/EtOH=70/30(V/V)

Flow rate: 25 mL/min

Wave length: UV 254 nm

Temperature: 35° C.

ESI-MS (EI⁺, m/z): 1036.4 [M+Na]⁺. ¹H NMR (400 MHz, CDCl₃) δ 6.44-5.80 (m, 4H), 5.65-5.01 (m, 4H), 4.64 (d, J=15.9 Hz, 1H), 3.99-3.52 (m, 11H), 3.47-3.02 (m, 16H), 3.02-2.46 (m, 5H), 2.43-1.85 (m, 8H), 1.83-1.64 (m, 9H), 1.46-1.19 (m, 10H), 1.16-0.83 (m, 18H), 0.79-0.59 (m, 1H).

### Example 16: Synthesis of (23E,25E,27E,28E,36R,37S,38R,39R,41S,43S,46S,47R,48R,57R)-57-hydroxy-46-[(1R)-2-[(1S,3R,4R)-4-hydroxy-3-methoxy-cyclohexyl]-1-methyl-ethyl]-47,48-dimethoxy-45-[2-[2-(2-methoxyethoxy)ethoxy]ethoxy]-36,37,38,39,49,50-hexamethyl-66,67-dioxa-58-azatricyclohexatriaconta-23,25,27(49),28(50)-tetraene-51,52,53,54,55-pentone (I-28)

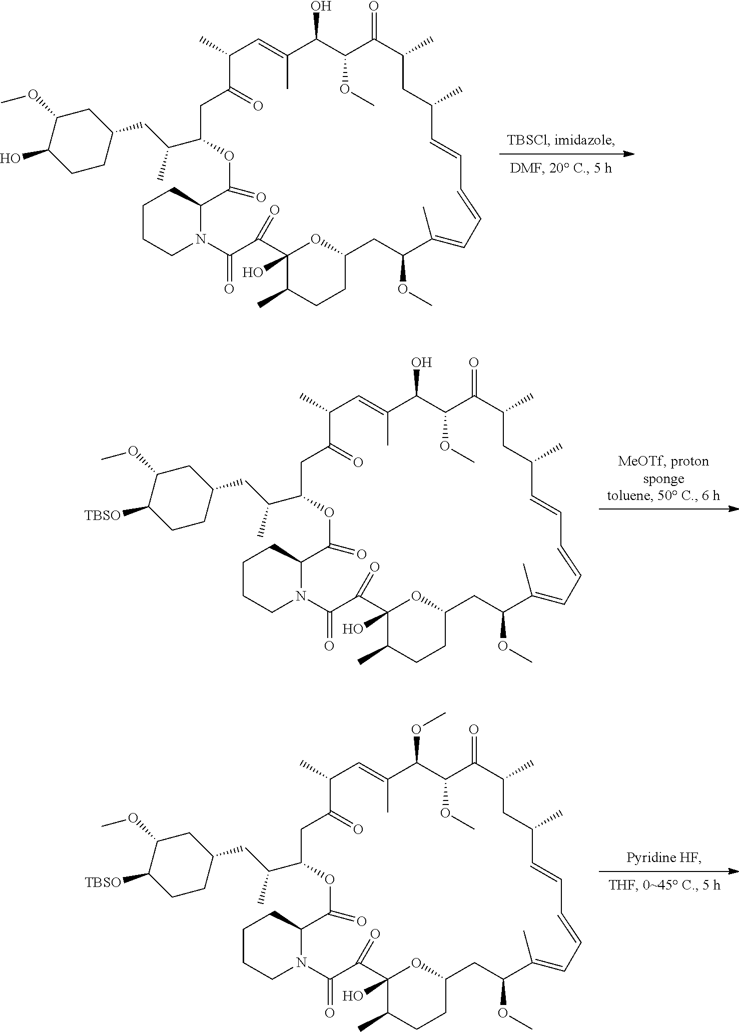

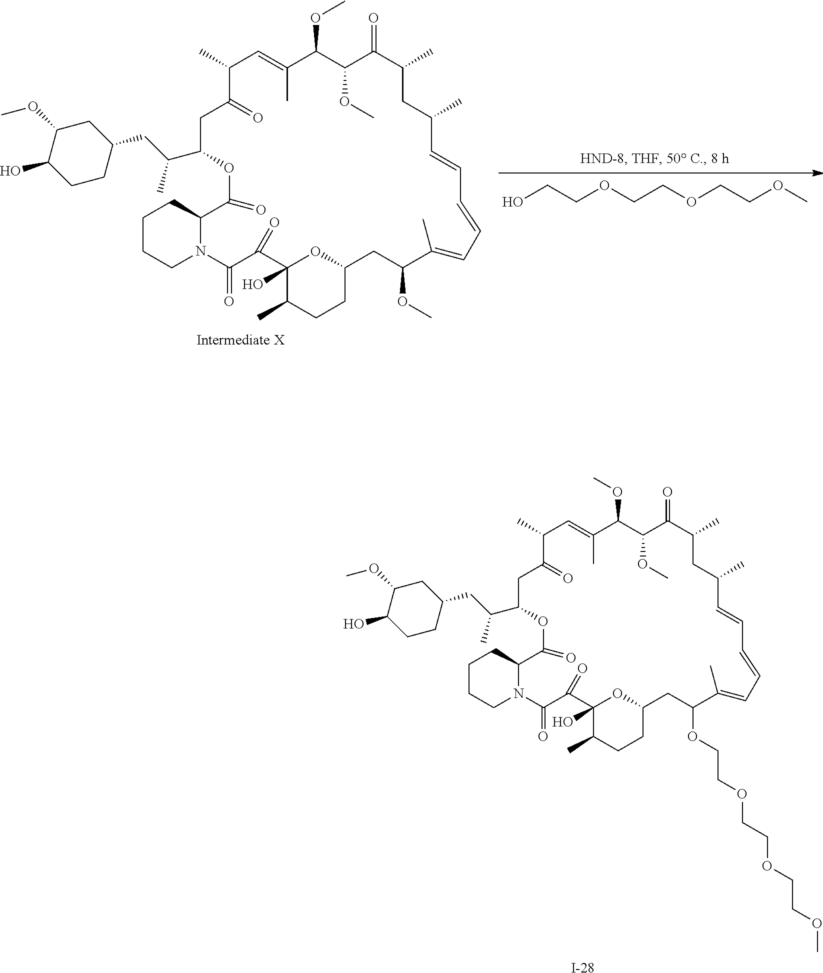

Step 1: (27E,29E,31E,32E,34R,35S,36R,37R,39S,41S,43S,44S,45R,46R,55R)-44-[(1R)-2-[(1S,3R,4R)-4-[tert-butyl(dimethyl)silyl]oxy-3-methoxy-cyclohexyl]-1-methyl-ethyl]-45,55-dihydroxy-43,46-dimethoxy-34,35,36,37,47,48-hexamethyl-65,66-dioxa-57-azatricyclohexatriaconta-27,29,31(47),32(48)-tetraene-49,50,51,52,53-pentone. To a solution of (22E,24E,26E,27E,29R,30S,31R,32R,34S,36S,38S,39S,40R,41R,50R)-40,50-dihydroxy-39-[(1R)-2-[(1S,3R,4R)-4-hydroxy-3-methoxy-cyclohexyl]-1-methyl-ethyl]-38,41-dimethoxy-29,30,31,32,42,43-hexamethyl-60,61-dioxa-51-azatricyclohexatriaconta-22,24,26(42),27(43)-tetraene-44,45,46,47,48-pentone (2 g, 2.19 mmol) in DMF (30 mL) was added imidazole (0.596 g, 8.75 mmol) and tert-butyl chlorodimethylsilane (0.989 g, 6.56 mmol) at rt. The mixture was stirred at 20° C. for 5 h then poured into ice cold saturated aqueous NH₄Cl solution (40 mL) and Et₂O:petroleum ether (60 mL, 2:1). The organic layer was washed with saturated aqueous NH₄Cl solution (20 mL), washed with water and brine (20 mL), dried over Na₂SO₄, filtered and concentrated. The residue was purified via silica gel chromatography (EtOAc in petroleum ether from 10% to 50%) to provide the titled compound (1.5 g, 67% yield) as a white solid. ESI-MS (EI⁺, m/z): 1049.8 [M+Na]⁺.

Step 2: (28E,30E,32E,33E,35R,36S,37R,38R,40S,42S,44S,45S,46R,47R,56R)-45-[(1R)-2-[(1S,3R,4R)-4-[tert-butyl(dimethyl)silyl]oxy-3-methoxy-cyclohexyl]-1-methyl-ethyl]-56-hydroxy-44,46,47-trimethoxy-35,36,37,38,48,49-hexamethyl-65,66-dioxa-58-azatricyclohexatriaconta-28,30,32(48),33(49)-tetraene-50,51,52,53,54-pentone. To a suspension of (27E,29E,31E,32E,34R,35S,36R,37R,39S,41S,43S,44S,45R,46R,55R)-44-[(1R)-2-[(1S,3R,4R)-4-[tert-butyl(dimethyl)silyl]oxy-3-methoxy-cyclohexyl]-1-methyl-ethyl]-45,55-dihydroxy-43,46-dimethoxy-34,35,36,37,47,48-hexamethyl-65,66-dioxa-57-azatricyclohexatriaconta-27,29,31(47),32(48)-tetraene-49,50,51,52,53-pentone (0.6 g, 0.58 mmol) and 1,8-bis(dimethylamino)naphtalene (1.5 g, 7 mmol) in toluene (20 mL) was added methyl trifluoromethanesulfonate (0.957 g, 5.83 mmol) dropwise at rt under N₂. After the addition, the mixture was heated to 50° C. for 6 h then cooled, filtered and the filtrate purified via silica gel chromatography (EtOAc:petroleum ether=4:1) to provide the titled compound (0.24 g, 39% yield) as a white solid. ESI-MS (EI⁺, m/z): 1063.8 [M+Na]⁺.

Step 3: (23E,25E,27E,28E,30R,31S,32R,33R,35S,37S,39S,40S,41R,42R,51R)-51-hydroxy-40-[(1R)-2-[(1S,3R,4R)-4-hydroxy-3-methoxy-cyclohexyl]-1-methyl-ethyl]-39,41,42-trimethoxy-30,31,32,33,43,44-hexamethyl-60,61-dioxa-52-azatricyclohexatriaconta-23,25,27(43),28(44)-tetraene-45,46,47,48,49-pentone. To a solution of (28E,30E,32E,33E,35R,36S,37R,38R,40S,42S,44S,45S,46R,47R,56R)-45-[(1R)-2-[(1S,3R,4R)-4-[tert-butyl(dimethyl)silyl]oxy-3-methoxy-cyclohexyl]-1-methyl-ethyl]-56-hydroxy-44,46,47-trimethoxy-35,36,37,38,48,49-hexamethyl-65,66-dioxa-58-azatricyclohexatriaconta-28,30,32(48),33(49)-tetraene-50,51,52,53,54-pentone (0.24 g, 0.23 mmol) in THF (10 mL) was added pyridine hydrofluoride (2.28 g, 23.02 mmol, 2 mL) at 0° C. The reaction was stirred at 45° C. for 5 h then diluted with DCM and saturated aqueous NaHCO₃ solution, washed with water and brine, dried over Na₂SO₄, filtered and concentrated. The residue was purified via reverse phase chromatography (78% CH₃CN in water) to provide the titled compound (0.105 g, 49% yield) as a white solid. ESI-MS (EI⁺, m/z): 949.7 [M+Na]⁺.

Step 4: (23E,25E,27E,28E,36R,37S,38R,39R,41S,43S,46S,47R,48R,57R)-57-hydroxy-46-[(1R)-2-[(1S,3R,4R)-4-hydroxy-3-methoxy-cyclohexyl]-1-methyl-ethyl]-47,48-dimethoxy-45-[2-[2-(2-methoxyethoxy)ethoxy]ethoxy]-36,37,38,39,49,50-hexamethyl-66,67-dioxa-58-azatricyclohexatriaconta-23,25,27(49),28(50)-tetraene-51,52,53,54,55-pentone (I-28). To a solution of (23E,25E,27E,28E,30R,31S,32R,33R,35S,37S,39S,40S,41R,42R,51R)-51-hydroxy-40-[(1R)-2-[(1S,3R,4R)-4-hydroxy-3-methoxy-cyclohexyl]-1-methyl-ethyl]-39,41,42-trimethoxy-30,31,32,33,43,44-hexamethyl-60,61-dioxa-52-azatricyclohexatriaconta-23,25,27(43),28(44)-tetraene-45,46,47,48,49-pentone (0.15 g, 0.16 mmol) in THF (15 mL) under nitrogen at 0° C. was added 2-(2-(2-methoxyethoxy)ethoxy)ethanol (0.265 g, 1.62 mmol) and HND-8 (0.3 g) and the mixture was stirred at 50° C. for 8 h. The reaction mixture was purified via reverse phase chromatography eluting with 80% CH₃CN in water then by preparative TLC (petroleum ether: ethyl acetate=1:2) to provide (23E,25E,27E,28E,36R,37S,38R,39R,41S,43S,46S,47R,48R,57R)-57-hydroxy-46-[(1R)-2-[(1S,3R,4R)-4-hydroxy-3-methoxy-cyclohexyl]-1-methyl-ethyl]-47,48-dimethoxy-45-[2-[2-(2-methoxyethoxy)ethoxy]ethoxy]-36,37,38,39,49,50-hexamethyl-66,67-dioxa-58-azatricyclohexatriaconta-23,25,27(49),28(50)-tetraene-51,52,53,54,55-pentone (36.5 mg, 21% yield) as a white solid. ESI-MS (EI⁺, m/z): 1035.8 [M+Na]⁺. ¹HNMR (500 MHz, CDCl₃): ¹H NMR (400 MHz, CDCl₃) δ 6.59-5.88 (m, 3H), 5.85-4.93 (m, 4H), 4.72-4.18 (m, 1H), 4.15-3.76 (m, 2H), 3.74-3.52 (m, 8H), 3.50-3.30 (m, 8H), 3.29-3.03 (m, 5H), 3.03-2.47 (m, 5H), 2.45-1.89 (m, 6H), 1.90-1.52 (m, 21H), 1.32 (ddd, J=28.1, 22.9, 5.8 Hz, 9H), 1.19-0.78 (m, 14H), 0.69 (d, J=12.0 Hz, 1H).

### Example 17: Synthesis of (24E,26E,28E,29E,35R,36S,37R,38R,40S,42S,45S,46R,47R,56R)-45-[(1R)-2-[(1S,3R,4R)-3,4-dimethoxycyclohexyl]-1-methyl-ethyl]-56-hydroxy-46,47-dimethoxy-44-[2-(2-methoxyethoxy)ethoxy]-35,36,37,38,48,49-hexamethyl-64,65-dioxa-57-azatricyclohexatriaconta-24,26,28(48),29(49)-tetraene-50,51,52,53,54-pentone (I-29), (24E,26E,28E,29E,35R,36S,37R,38R,40S,42S,44S,45S,46R,47R,56R)-45-[(1R)-2-[(1S,3R,4R)-3,4-dimethoxycyclohexyl]-1-methyl-ethyl]-56-hydroxy-46,47-dimethoxy-44-[2-(2-methoxyethoxy)ethoxy]-35,36,37,38,48,49-hexamethyl-64,65-dioxa-57-azatricyclohexatriaconta-24,26,28(48),29(49)-tetraene-50,51,52,53,54-pentone (I-31) and (24E,26E,28E,29E,35R,36S,37R,38R,40S,42S,44R,45S,46R,47R,56R)-45-[(1R)-2-[(1S,3R,4R)-3,4-dimethoxycyclohexyl]-1-methyl-ethyl]-56-hydroxy-46,47-dimethoxy-44-[2-(2-methoxyethoxy)ethoxy]-35,36,37,38,48,49-hexamethyl-64,65-dioxa-57-azatricyclohexatriaconta-24,26,28(48),29(49)-tetraene-50,51,52,53,54-pentone (I-30)

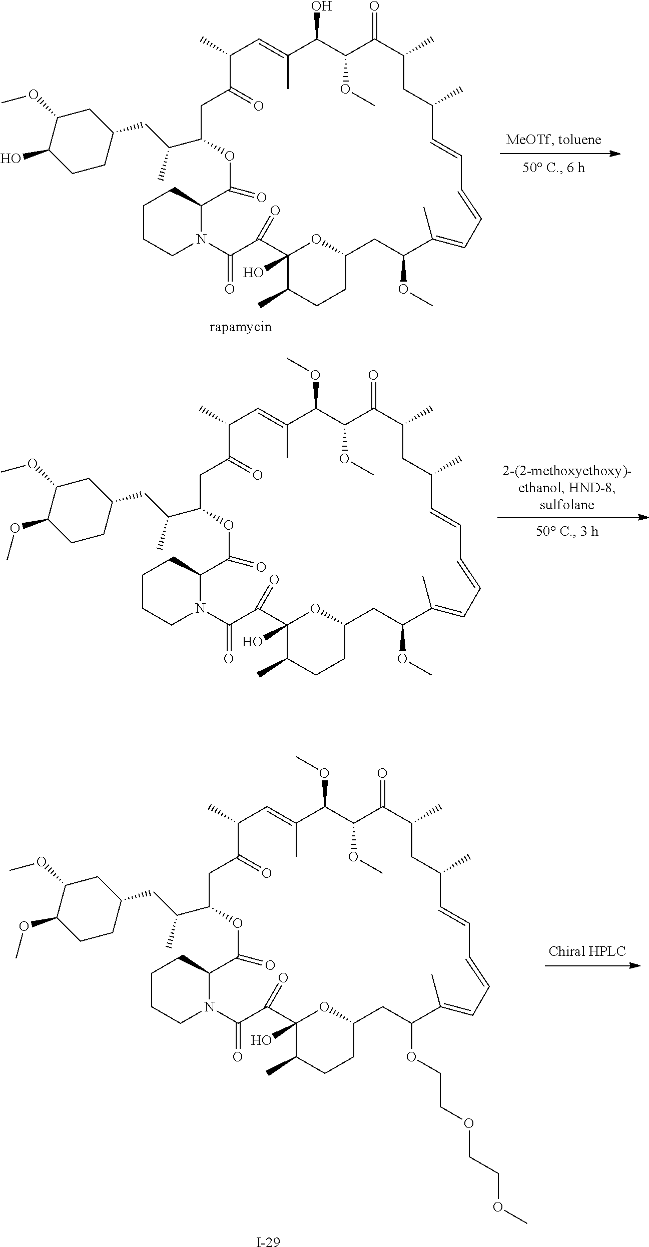

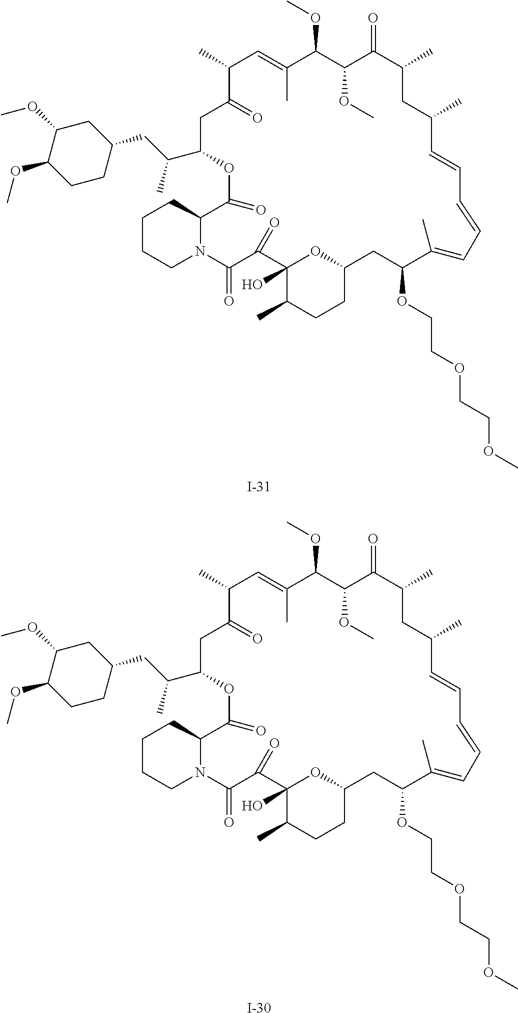

Step 1: (24E,26E,28E,29E,31R,32S,33R,34R,36S,38S,40S,41S,42R,43R,52R)-41-[(1R)-2-[(1S,3R,4R)-3,4-dimethoxycyclohexyl]-1-methyl-ethyl]-52-hydroxy-40,42,43-trimethoxy-31,32,33,34,44,45-hexamethyl-60,61-dioxa-53-azatricyclohexatriaconta-24,26,28(44),29(45)-tetraene-46,47,48,49,50-pentone. To a solution of (22E,24E,26E,27E,29R,30S,31R,32R,34S,36S,38S,39S,40R,41R,50R)-40,50-dihydroxy-39-[(1R)-2-[(1S,3R,4R)-4-hydroxy-3-methoxy-cyclohexyl]-1-methyl-ethyl]-38,41-dimethoxy-29,30,31,32,42,43-hexamethyl-60,61-dioxa-51-azatricyclohexatriaconta-22,24,26(42),27(43)-tetraene-44,45,46,47,48-pentone (0.2 g, 0.219 mmol) in toluene (5 mL) was added proton sponge (0.938 g, 4.38 mmol) followed by methyl trifluoromethanesulfonate (0.539 g, 3.28 mmol) at rt. The mixture was stirred at 50° C. for 6 hrs, cooled and purified by silica gel chromatography and then by reverse phase chromatography (85% CH₃CN in water) to provide the titled compound (50 mg, 24% yield) as a white solid. ESI-MS (EI⁺, m/z): 964.2 [M+Na]⁺. ¹H NMR (400 MHz, CDCl₃) δ 6.50-5.80 (m, 4H), 5.62 (ddd, J=22.9, 14.5, 7.9 Hz, 1H), 5.32 (dt, J=11.6, 7.7 Hz, 2H), 5.18-5.03 (m, 1H), 4.68 (s, 1H), 3.95-3.54 (m, 5H), 3.50-3.33 (m, 7H), 3.32-3.21 (m, 3H), 3.18-2.92 (m, 8H), 2.83-2.48 (m, 3H), 2.25 (dd, J=30.1, 10.7 Hz, 2H), 2.02 (ddd, J=34.0, 26.3, 9.6 Hz, 4H), 1.88-1.56 (m, 14H), 1.51-1.16 (m, 9H), 1.15-0.82 (m, 18H), 0.79-0.68 (m, 1H).

Step 2: (24E,26E,28E,29E,35R,36S,37R,38R,40S,42S,45S,46R,47R,56R)-45-[(1R)-2-[(1S,3R,4R)-3,4-dimethoxycyclohexyl]-1-methyl-ethyl]-56-hydroxy-46,47-dimethoxy-44-[2-(2-methoxyethoxy)ethoxy]-35,36,37,38,48,49-hexamethyl-64,65-dioxa-57-azatricyclohexatriaconta-24,26,28(48),29(49)-tetraene-50,51,52,53,54-pentone (I-29). To a solution of (24E,26E,28E,29E,31R,32S,33R,34R,36S,38S,40S,41S,42R,43R,52R)-41-[(1R)-2-[(1S,3R,4R)-3,4-dimethoxycyclohexyl]-1-methyl-ethyl]-52-hydroxy-40,42,43-trimethoxy-31,32,33,34,44,45-hexamethyl-60,61-dioxa-53-azatricyclohexatriaconta-24,26,28(44),29(45)-tetraene-46,47,48,49,50-pentone (0.17 g, 0.18 mmol) and 2-(2-methoxyethoxy)ethanol (0.43 g, 3.61 mmol) in sulfolane (5 mL) was added HND-8 (35 mg) at 50° C. under N₂. The resulting solution was stirred at 50° C. for 3 hrs, filtered and the filtrate was passed through a C18 column, eluting with 85% CH₃CN in water to provide I-29 (65 mg, 35% yield) as a white solid. ESI-MS (EI⁺, m/z): 1052.5 [M+Na]⁺. ¹H NMR (500 MHz, CDCl₃) δ 6.46-5.81 (m, 4H), 5.74-5.03 (m, 4H), 4.68-4.15 (m, 2H), 3.99-3.52 (m, 11H), 3.50-3.22 (m, 16H), 3.21-2.98 (m, 6H), 2.94-2.44 (m, 3H), 2.37-1.89 (m, 7H), 1.86-1.69 (m, 7H), 1.52-1.24 (m, 9H), 1.22-0.84 (m, 21H), 0.74 (dd, J=22.3, 10.9 Hz, 1H).

Step 3: (24E,26E,28E,29E,35R,36S,37R,38R,40S,42S,44S,45S,46R,47R,56R)-45-[(1R)-2-[(1S,3R,4R)-3,4-dimethoxycyclohexyl]-1-methyl-ethyl]-56-hydroxy-46,47-dimethoxy-44-[2-(2-methoxyethoxy)ethoxy]-35,36,37,38,48,49-hexamethyl-64,65-dioxa-57-azatricyclohexatriaconta-24,26,28(48),29(49)-tetraene-50,51,52,53,54-pentone (I-31) and (24E,26E,28E,29E,35R,36S,37R,38R,40S,42S,44R,45S,46R,47R,56R)-45-[(1R)-2-[(1S,3R,4R)-3,4-dimethoxycyclohexyl]-1-methyl-ethyl]-56-hydroxy-46,47-dimethoxy-44-[2-(2-methoxyethoxy)ethoxy]-35,36,37,38,48,49-hexamethyl-64,65-dioxa-57-azatricyclohexatriaconta-24,26,28(48),29(49)-tetraene-50,51,52,53,54-pentone (I-30). 130 mg of the mixture was separated via chiral preparative HPLC then purified via silica gel chromatography (hexane:DCM:EtOAc:MeOH from 3:3:1:0 to 3:3:1:0.4) to provide I-31 (45 mg, 35% yield) as a white solid and I-30 (40 mg, 31% yield) as a white solid.

Chiral separation method:

Column: CHIRALPAK IC

Column size: 5.0 cm I.D.×25 cm L, 10 μm

Sample solution: 1.4 mg/mL in Mobile phase

Injection: 15 mL

Mobile phase: Hexane/EtOH=50/50(V/V)

Flow rate: 60 mL/min

Wave length: UV 254 nm

Temperature: 35° C.

I-31: ESI-MS (EI⁺, m/z): 1052.1 [M+Na]⁺. ¹H NMR (400 MHz, CDCl₃) δ 6.51-5.82 (m, 4H), 5.76-5.03 (m, 4H), 4.51 (dd, J=56.0, 27.4 Hz, 1H), 4.35-4.06 (m, 1H), 4.00-3.20 (m, 26H), 3.19-2.98 (m, 5H), 2.88-2.48 (m, 3H), 2.40-1.85 (m, 7H), 1.82-1.65 (m, 11H), 1.38 (ddd, J=37.8, 31.6, 21.3 Hz, 10H), 1.21-0.83 (m, 18H), 0.79-0.68 (m, 1H).
I-30: ESI-MS (EI⁺, m/z): 1052.2 [M+Na]⁺. ¹H NMR (400 MHz, CDCl₃) δ 6.52-5.81 (m, 4H), 5.77-5.04 (m, 5H), 4.70-4.14 (m, 2H), 4.01-2.97 (m, 31H), 2.64 (dd, J=50.7, 36.3 Hz, 3H), 2.42-1.68 (m, 16H), 1.50-0.61 (m, 30H).

### Example 18: Synthesis of (25E,27E,29E,30E,36R,37S,38R,39R,41S,43S,46S,47R,48R,57R)-46-[(1R)-2-[(1S,3R,4R)-4-dimethylphosphoryloxy-3-methoxy-cyclohexyl]-1-methyl-ethyl]-57-hydroxy-47,48-dimethoxy-45-[2-(2-methoxyethoxy)ethoxy]-36,37,38,39,49,50-hexamethyl-66,67-dioxa-58-azatricyclohexatriaconta-25,27,29(49),30(50)-tetraene-51,52,53,54,55-pentone (I-32), (25E,27E,29E,30E,36R,37S,38R,39R,41S,43S,45S,46S,47R,48R,57R)-46-[(1R)-2-[(1S,3R,4R)-4-dimethylphosphoryloxy-3-methoxy-cyclohexyl]-1-methyl-ethyl]-57-hydroxy-47,48-dimethoxy-45-[2-(2-methoxyethoxy)ethoxy]-36,37,38,39,49,50-hexamethyl-66,67-dioxa-58-azatricyclohexatriaconta-25,27,29(49),30(50)-tetraene-51,52,53,54,55-pentone (I-34) and (25E,27E,29E,30E,36R,37S,38R,39R,41S,43S,45R,46S,47R,48R,57R)-46-[(1R)-2-[(1S,3R,4R)-4-dimethylphosphoryloxy-3-methoxy-cyclohexyl]-1-methyl-ethyl]-57-hydroxy-47,48-dimethoxy-45-[2-(2-methoxyethoxy)ethoxy]-36,37,38,39,49,50-hexamethyl-66,67-dioxa-58-azatricyclohexatriaconta-25,27,29(49),30(50)-tetraene-51,52,53,54,55-pentone (I-33)

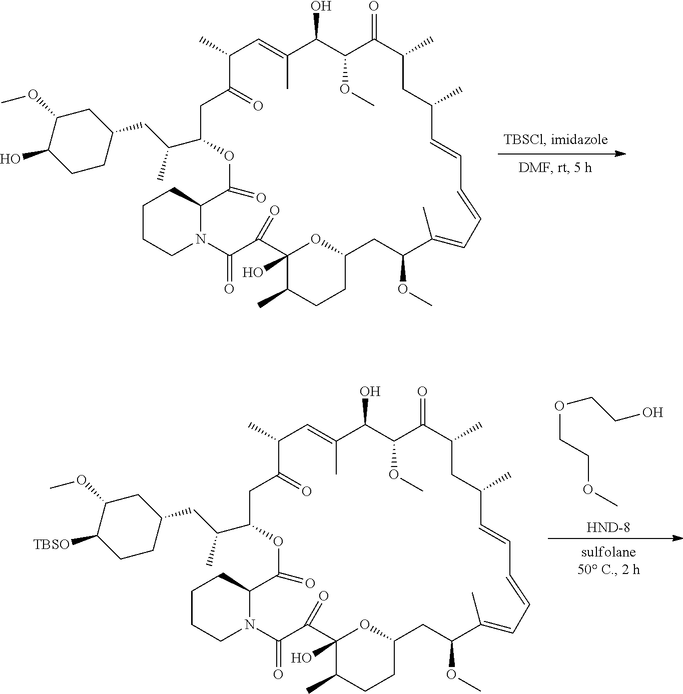

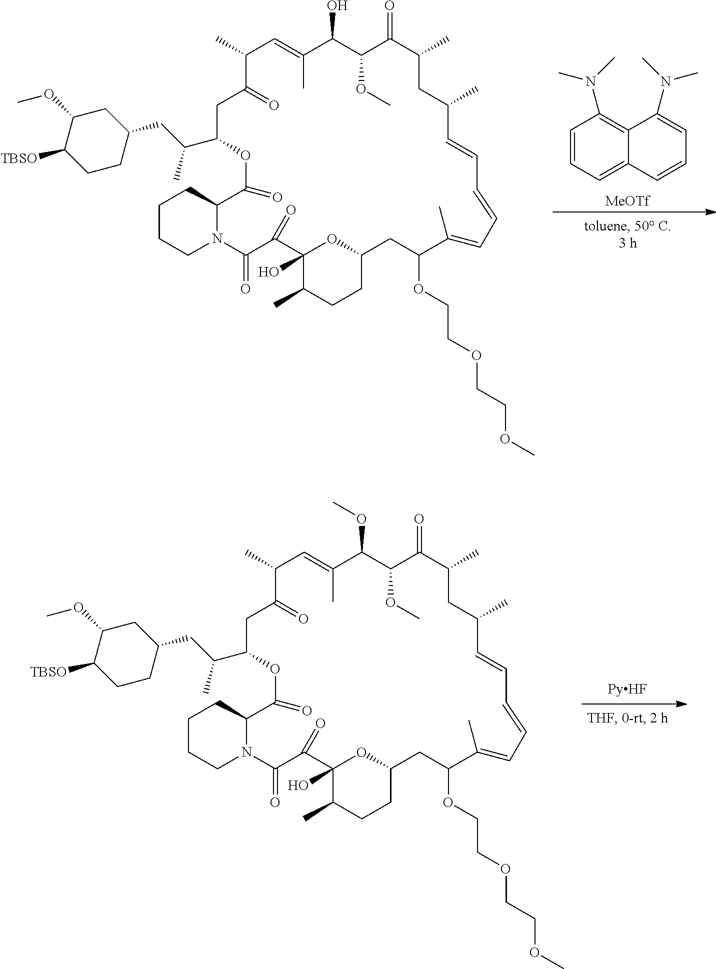

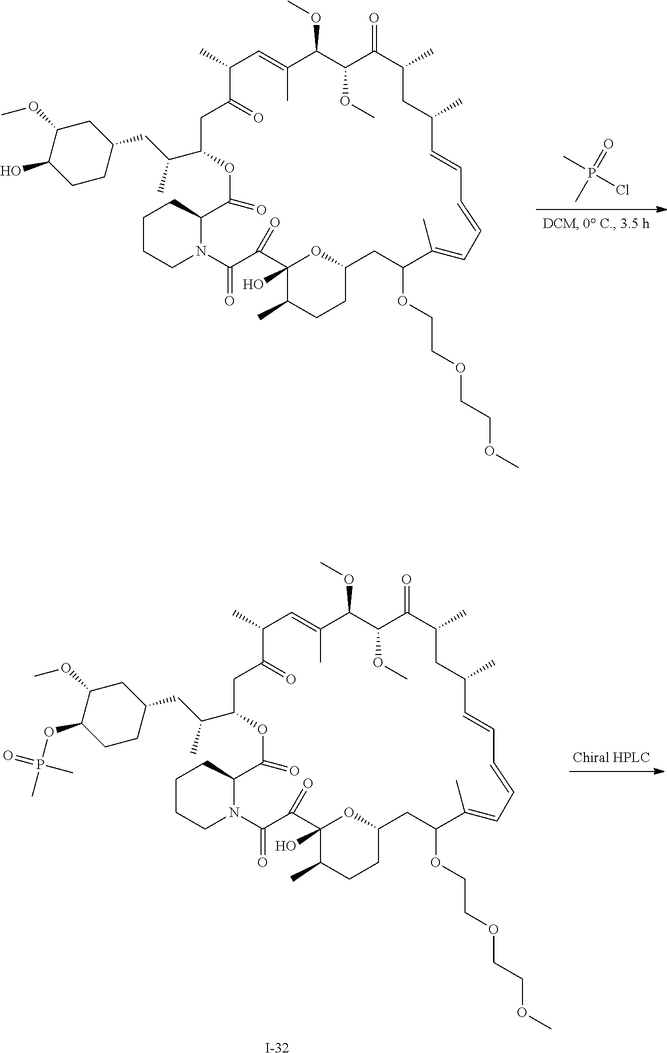

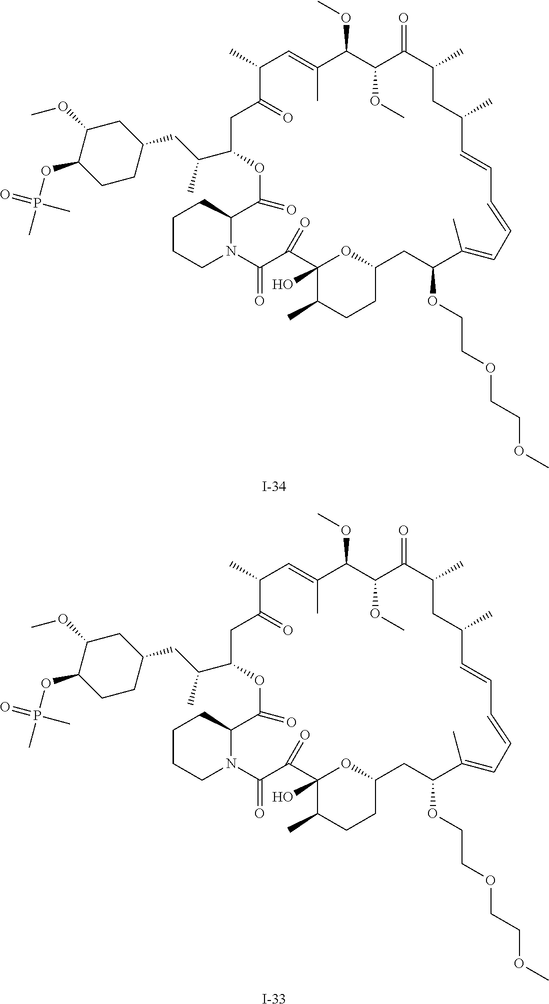

Step 1: (27E,29E,31E,32E,34R,35S,36R,37R,39S,41S,43S,44S,45R,46R,55R)-44-[(1R)-2-[(1S,3R,4R)-4-[tert-butyl(dimethyl)silyl]oxy-3-methoxy-cyclohexyl]-1-methyl-ethyl]-45,55-dihydroxy-43,46-dimethoxy-34,35,36,37,47,48-hexamethyl-65,66-dioxa-57-azatricyclohexatriaconta-27,29,31(47),32(48)-tetraene-49,50,51,52,53-pentone. To a solution of (22E,24E,26E,27E,29R,30S,31R,32R,34S,36S,38S,39S,40R,41R,50R)-40,50-dihydroxy-39-[(1R)-2-[(1S,3R,4R)-4-hydroxy-3-methoxy-cyclohexyl]-1-methyl-ethyl]-38,41-dimethoxy-29,30,31,32,42,43-hexamethyl-60,61-dioxa-51-azatricyclohexatriaconta-22,24,26(42),27(43)-tetraene-44,45,46,47,48-pentone (2 g, 2.19 mmol) in DMF (15 mL) was added imidazole (0.298 g, 4.38 mmol) and tert-butyl-chloro-dimethyl-silane (0.495 g, 3.28 mmol). The mixture was stirred at 20° C. for 5 h then poured into ice cold saturated aqueous NH₄Cl solution (10 mL) and Et₂O:petroleum ether (300 mL, 2:1), the organic layer was washed with saturated aqueous NH₄Cl solution (100 mL), washed with water and brine (100 mL), dried over Na₂SO₄, filtered and concentrated. The residue was purified by silica gel chromatography (EtOAc in petroleum ether from 10% to 50%) to provide the titled compound (1.85 g, 82% yield) as a white solid. ESI-MS (EI⁺, m/z): 1050.2 [M+Na]⁺. ¹H NMR (400 MHz, CDCl₃) δ 6.44-5.83 (m, 4H), 5.60-5.07 (m, 4H), 4.32-4.04 (m, 2H), 3.79 (d, J=13.4 Hz, 1H), 3.70 (d, J=6.1 Hz, 1H), 3.65 (dd, J=9.8, 5.5 Hz, 1H), 3.62-3.53 (m, 1H), 3.43-3.28 (m, 8H), 3.13 (s, 3H), 2.94-2.81 (m, 1H), 2.73 (dd, J=16.8, 5.9 Hz, 2H), 2.63-2.47 (m, 1H), 2.33 (d, J=12.7 Hz, 2H), 2.07-1.89 (m, 4H), 1.89-1.40 (m, 19H), 1.38-1.02 (m, 15H), 1.02-0.76 (m, 18H), 0.69 (s, 1H), 0.05 (dd, J=8.2, 5.1 Hz, 6H).

Step 2: (27E,29E,31E,32E,38R,39S,40R,41R,43S,45S,48S,49R,50R,59R)-48-[(1R)-2-[(1S,3R,4R)-4-[tert-butyl(dimethyl)silyl]oxy-3-methoxy-cyclohexyl]-1-methyl-ethyl]-49,59-dihydroxy-50-methoxy-47-[2-(2-methoxyethoxy)ethoxy]-38,39,40,41,51,52-hexamethyl-69,70-dioxa-61-azatricyclohexatriaconta-27,29,31(51),32(52)-tetraene-53,54,55,56,57-pentone. To a solution of (27E,29E,31E,32E,34R,35S,36R,37R,39S,41S,43S,44S,45R,46R,55R)-44-[(1R)-2-[(1S,3R,4R)-4-[tert-butyl(dimethyl)silyl]oxy-3-methoxy-cyclohexyl]-1-methyl-ethyl]-45,55-dihydroxy-43,46-dimethoxy-34,35,36,37,47,48-hexamethyl-65,66-dioxa-57-azatricyclohexatriaconta-27,29,31(47),32(48)-tetraene-49,50,51,52,53-pentone (1.7 g, 1.65 mmol) and 2-(2-methoxyethoxy)ethanol (3.97 g, 33.06 mmol) in sulfolane (20 mL) was added HND-8 (255 mg) at 50° C. under N₂, the resulting solution was then stirred at 50° C. for 2 hrs. The reaction mixture was poured into water, extracted with EtOAc, washed with water and brine, dried over Na₂SO₄, filtered and concentrated. The residue was purified by silica gel chromatography (50% EtOAc in petroleum ether) and then by reverse phase chromatography (85% CH₃CN in water) to provide the titled compound (950 mg, 51% yield) as a white solid. ESI-MS (EI⁺, m/z): 1138.2 [M+Na]⁺.

Step 3: (28E,30E,32E,33E,39R,40S,41R,42R,44S,46S,49S,50R,51R,60R)-49-[(1R)-2-[(1S,3R,4R)-4-[tert-butyl(dimethyl)silyl]oxy-3-methoxy-cyclohexyl]-1-methyl-ethyl]-60-hydroxy-50,51-dimethoxy-48-[2-(2-methoxyethoxy)ethoxy]-39,40,41,42,52,53-hexamethyl-69,70-dioxa-62-azatricyclohexatriaconta-28,30,32(52),33(53)-tetraene-54,55,56,57,58-pentone. To a solution of (27E,29E,31E,32E,38R,39S,40R,41R,43S,45S,48S,49R,50R,59R)-48-[(1R)-2-[(1S,3R,4R)-4-[tert- butyl(dimethyl)silyl]oxy-3-methoxy-cyclohexyl]-1-methyl-ethyl]-49,59-dihydroxy-50-methoxy-47-[2-(2-methoxyethoxy)ethoxy]-38,39,40,41,51,52-hexamethyl-69,70-dioxa-61-azatricyclohexatriaconta-27,29,31(51),32(52)-tetraene-53,54,55,56,57-pentone (0.5 g, 0.448 mmol) in toluene (15 mL) was added N1,N1,N8,N8-tetramethylnaphthalene-1,8-diamine (1.92 g, 8.96 mmol) and methyl trifluoromethanesulfonate (1.10 g, 6.72 mmol) at rt. The resulting solution was stirred at 50° C. for 3 h then filtered and concentrated. The residue was purified via silica-gel chromatography, eluting with EtOAc in petroleum ether from 0% to 50% then by reverse phase chromatography (CH₃CN in water from 0% to 100%) to provide the titled compound (160 mg, 32% yield) as a light yellow solid. ESI-MS (EI⁺, m/z): 1152.2 [M+Na]⁺.

Step 4: (23E,25E,27E,28E,34R,35S,36R,37R,39S,41S,44S,45R,46R,55R)-55-hydroxy-44-[(1R)-2-[(1S,3R,4R)-4-hydroxy-3-methoxy-cyclohexyl]-1-methyl-ethyl]-45,46-dimethoxy-43-[2-(2-methoxyethoxy)ethoxy]-34,35,36,37,47,48-hexamethyl-64,65-dioxa-56-azatricyclohexatriaconta-23,25,27(47),28(48)-tetraene-49,50,51,52,53-pentone. To a solution of (28E,30E,32E,33E,39R,40S,41R,42R,44S,46S,49S,50R,51R,60R)-49-[(1R)-2-[(1S,3R,4R)-4-[tert- butyl(dimethyl)silyl]oxy-3-methoxy-cyclohexyl]-1-methyl-ethyl]-60-hydroxy-50,51-dimethoxy-48-[2-(2-methoxyethoxy)ethoxy]-39,40,41,42,52,53-hexamethyl-69,70-dioxa-62-azatricyclohexatriaconta-28,30,32(52),33(53)-tetraene-54,55,56,57,58-pentone (0.58 g, 0.513 mmol) in THF (20 mL) was added Py.HF (2.54 g, 25.65 mmol) at 0° C. The reaction was stirred at rt for 3 hrs then diluted with DCM and saturated aqueous NaHCO₃ solution, washed with water and brine, dried over Na₂SO₄, filtered and concentrated. The residue was purified via reverse phase chromatography (75% CH₃CN in water) to provide the titled compound (200 mg, 38% yield) as a white solid. ESI-MS (EI⁺, m/z): 1038.1 [M+Na]⁺. ¹H NMR (400 MHz, CDCl₃) δ 6.49-5.79 (m, 4H), 5.69-5.03 (m, 4H), 4.62 (d, J=13.2 Hz, 1H), 4.00-3.07 (m, 28H), 3.02-2.47 (m, 6H), 2.41-1.68 (m, 16H), 1.54-1.21 (m, 11H), 1.17-0.82 (m, 18H), 0.79-0.55 (m, 1H).

Step 5: (25E,27E,29E,30E,36R,37S,38R,39R,41S,43S,46S,47R,48R,57R)-46-[(1R)-2-[(1S,3R,4R)-4-dimethylphosphoryloxy-3-methoxy-cyclohexyl]-1-methyl-ethyl]-57-hydroxy-47,48-dimethoxy-45-[2-(2-methoxyethoxy)ethoxy]-36,37,38,39,49,50-hexamethyl-66,67-dioxa-58-azatricyclohexatriaconta-25,27,29(49),30(50)-tetraene-51,52,53,54,55-pentone (I-32). To a solution of (23E,25E,27E,28E,34R,35S,36R,37R,39S,41S,44S,45R,46R,55R)-55-hydroxy-44-[(1R)-2-[(1S,3R,4R)-4-hydroxy-3-methoxy-cyclohexyl]-1-methyl-ethyl]-45,46-dimethoxy-43-[2-(2-methoxyethoxy)ethoxy]-34,35,36,37,47,48-hexamethyl-64,65-dioxa-56-azatricyclohexatriaconta-23,25,27(47),28(48)-tetraene-49,50,51,52,53-pentone (0.18 g, 0.177 mmol) in DCM (3 mL) was added 2,6-di-tert-butyl-4-methylpyridine (0.273 g, 1.33 mmol) and dimethylphosphinic chloride (0.1 g, 0.89 mmol, dissolved in 0.5 mL of DCM) at 0° C. The resulting solution was stirred at 0° C. for 3.5 hrs, then diluted with EtOAc, washed with saturated aqueous NaHCO₃ solution, washed with water and brine, dried over Na₂SO₄, filtered and concentrated. The residue was purified by reverse phase chromatography (CH₃CN in water) to provide I-32 (90 mg, 47% yield) as a white solid. ESI-MS (EI⁺, m z): 1114.1 [M+Na]⁺. ¹H NMR (500 MHz, CDCl₃) δ 6.49-5.81 (m, 4H), 5.74-4.96 (m, 4H), 4.67-4.03 (m, 2H), 4.00-3.01 (m, 29H), 2.99-2.46 (m, 4H), 2.44-1.73 (m, 17H), 1.59-1.22 (m, 15H), 1.19-0.83 (m, 18H), 0.82-0.59 (m, 1H).

Step 6: (25E,27E,29E,30E,36R,37S,38R,39R,41S,43S,45S,46S,47R,48R,57R)-46-[(1R)-2-[(1S,3R,4R)-4-dimethylphosphoryloxy-3-methoxy-cyclohexyl]-1-methyl-ethyl]-57-hydroxy-47,48-dimethoxy-45-[2-(2-methoxyethoxy)ethoxy]-36,37,38,39,49,50-hexamethyl-66,67-dioxa-58-azatricyclohexatriaconta-25,27,29(49),30(50)-tetraene-51,52,53,54,55-pentone (I-34) and (25E,27E,29E,30E,36R,37S,38R,39R,41S,43S,45R,46S,47R,48R,57R)-46-[(1R)-2-[(1S,3R,4R)-4-dimethylphosphoryloxy-3-methoxy-cyclohexyl]-1-methyl-ethyl]-57-hydroxy-47,48-dimethoxy-45-[2-(2-methoxyethoxy)ethoxy]-36,37,38,39,49,50-hexamethyl-66,67-dioxa-58-azatricyclohexatriaconta-25,27,29(49),30(50)-tetraene-51,52,53,54,55-pentone (I-33). 125 mg of the epimeric mixture was separated via chiral preparative HPLC then purified via silica gel chromatography (hexane:DCM:EtOAc:MeOH from 3:3:1:0 to 3:3:1:0.3) to provide I-34 (25 mg, 20% yield) as a white solid and I-33 (15 mg, 12% yield) as a white solid.

Chiral separation method:

Column: CHIRALPAK IC

Column size: 5.0 cm I.D.×25 cm L, 10 μm

Sample solution: 1.2 mg/mL in Mobile phase

Injection: 10 mL

Mobile phase: Hexane/EtOH=40/60(V/V)

Flow rate: 25 mL/min

Wave length: UV 254 nm

Temperature: 35° C.

I-34: ESI-MS (EI⁺, m/z): 1114.1 [M+Na]⁺. ¹H NMR (500 MHz, CDCl₃) δ 6.41-6.01 (m, 3H), 5.87 (dd, J=83.6, 10.7 Hz, 1H), 5.57-5.40 (m, 1H), 5.38-4.97 (m, 3H), 4.57 (s, 1H), 4.02 (d, J=20.9 Hz, 1H), 3.92-3.62 (m, 3H), 3.61-2.94 (m, 26H), 2.78-2.40 (m, 3H), 2.29-1.79 (m, 9H), 1.60-1.38 (m, 15H), 1.36-1.11 (m, 9H), 1.08-0.76 (m, 18H), 0.75-0.64 (m, 1H).
I-33: ESI-MS (EI⁺, m/z): 1114.1 [M+Na]⁺. ¹H NMR (500 MHz, CDCl₃) δ 6.48-5.79 (m, 4H), 5.63-5.02 (m, 4H), 4.56 (d, J=62.6 Hz, 1H), 3.99-3.09 (m, 28H), 3.01-2.49 (m, 5H), 2.40-1.72 (m, 18H), 1.54-1.19 (m, 14H), 1.18-0.81 (m, 19H), 0.78-0.59 (m, 1H).

### Example 19: Synthesis of (23E,25E,27E,28E,36R,37S,38R,39R,41S,43S,46S,47R,48R,57R)-57-hydroxy-46-[(1R)-2-[(1S,3R,4R)-4-(2-hydroxyethoxy)-3-methoxy-cyclohexyl]-1-methyl-ethyl]-47,48-dimethoxy-45-[2-(2-methoxyethoxy)ethoxy]-36,37,38,39,49,50-hexamethyl-66,67-dioxa-58-azatricyclohexatriaconta-23,25,27(49),28(50)-tetraene-51,52,53,54,55-pentone (I-35), (23E,25E,27E,28E,36R,37S,38R,39R,41S,43S,45S,46S,47R,48R,57R)-57-hydroxy-46-[(1R)-2-[(1S,3R,4R)-4-(2-hydroxyethoxy)-3-methoxy-cyclohexyl]-1-methyl-ethyl]-47,48-dimethoxy-45-[2-(2-methoxyethoxy)ethoxy]-36,37,38,39,49,50-hexamethyl-66,67-dioxa-58-azatricyclohexatriaconta-23,25,27(49),28(50)-tetraene-51,52,53,54,55-pentone (I-37) and (23E,25E,27E,28E,36R,37S,38R,39R,41S,43S,45R,46S,47R,48R,57R)-57-hydroxy-46-[(1R)-2-[(1S,3R,4R)-4-(2-hydroxyethoxy)-3-methoxy-cyclohexyl]-1-methyl-ethyl]-47,48-dimethoxy-45-[2-(2-methoxyethoxy)ethoxy]-36,37,38,39,49,50-hexamethyl-66,67-dioxa-58-azatricyclohexatriaconta-23,25,27(49),28(50)-tetraene-51,52,53,54,55-pentone (I-36)

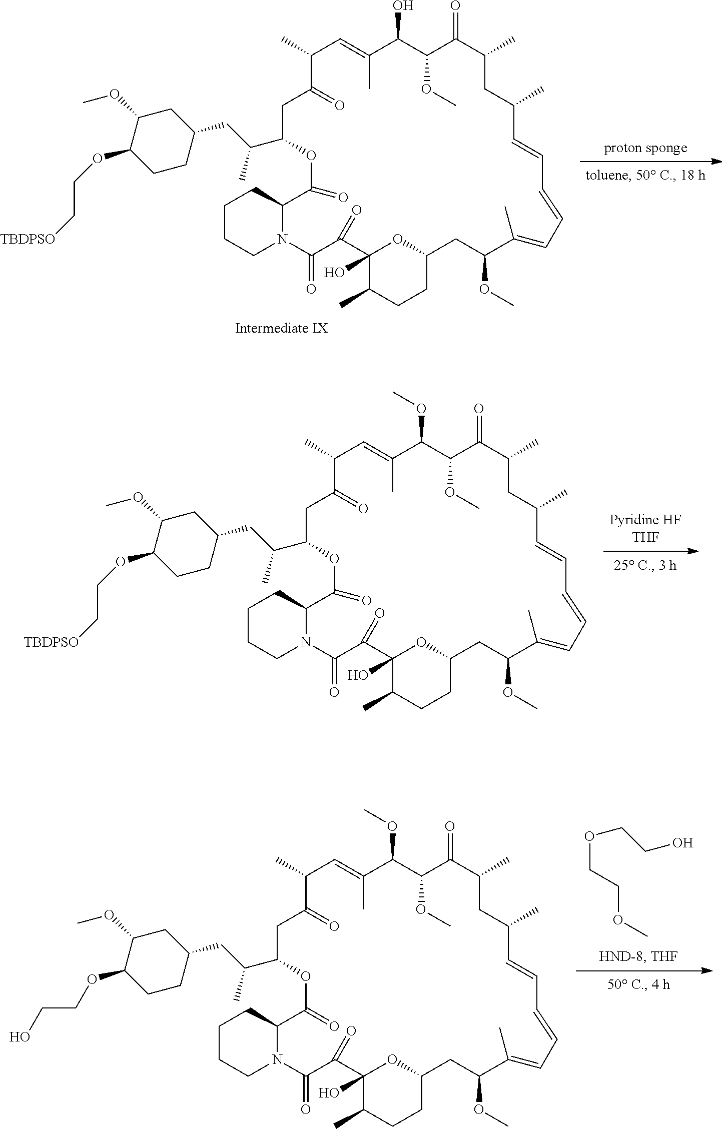

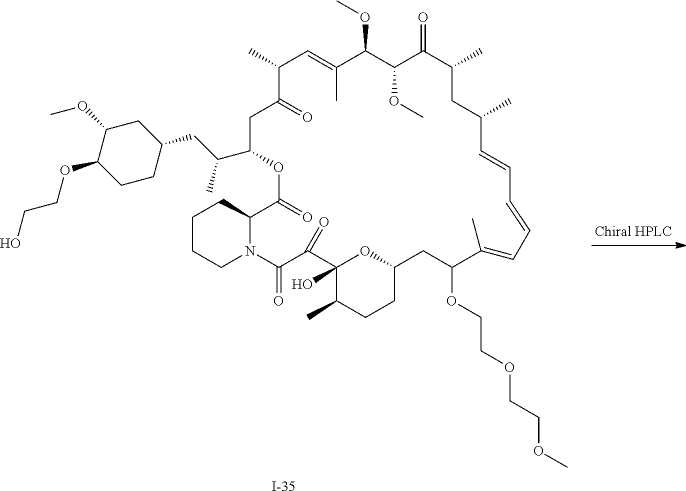

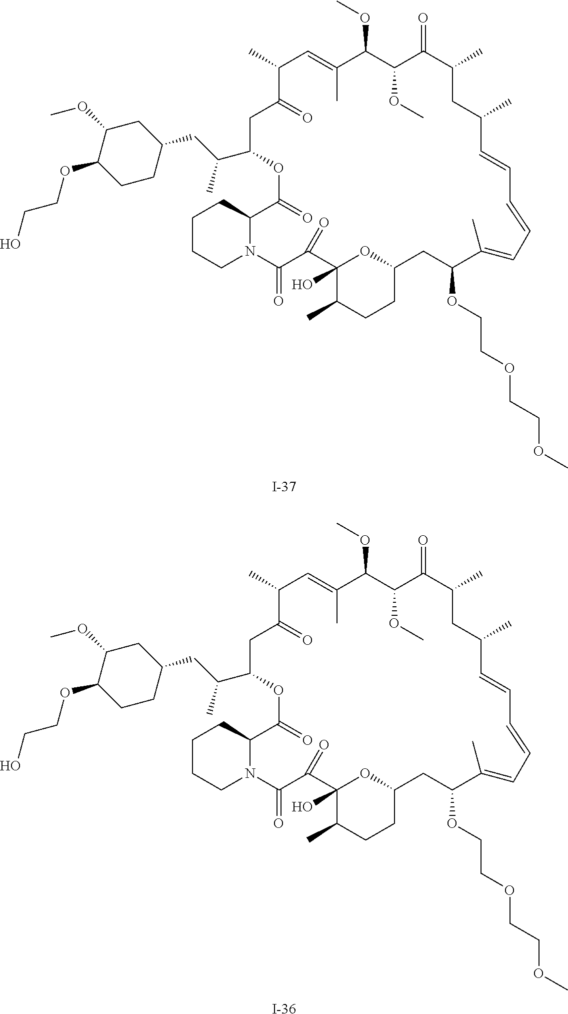

Step 1: (36E,38E,40E,41E,47R,48S,49R,50R,52S,54S,56S,57S,58R,59R,68R)-57-[(1R)-2-[(1S,3R,4R)-4-[2-[tert-butyl(diphenyl)silyl]oxyethoxy]-3-methoxy-cyclohexyl]-1-methyl-ethyl]-68-hydroxy-56,58,59-trimethoxy-47,48,49,50,60,61-hexamethyl-77,78-dioxa-70-azatricyclohexatriaconta-36,38,40(60),41(61)-tetraene-62,63,64,65,66-pentone. To a suspension of (35E,37E,39E,40E,46R,47S,48R,49R,51S,53S,55S,56S,57R,58R,67R)-56-[(1R)-2-[(1S,3R,4R)-4-[2-[tert-butyl(diphenyl)silyl]oxyethoxy]-3-methoxy-cyclohexyl]-1-methyl-ethyl]-57,67-dihydroxy-55,58-dimethoxy-46,47,48,49,59,60-hexamethyl-77,78-dioxa-69-azatricyclohexatriaconta-35,37,39(59),40(60)-tetraene-61,62,63,64,65-pentone (Intermediate IX was prepared according to Example 22, 1.8 g, 1.5 mmol) and 1,8-bis(dimethylamino)naphtalene (6.45 g, 30.08 mmol) in toluene (40 mL) was added methyl trifluoromethanesulfonate (3.70 g, 22.56 mmol) dropwise at rt under N₂. After addition, the mixture was heated to 50° C. for 5 hrs then the mixture was quenched by adding water (50 mL) and extracted with EtOAc (50 mL) at 0° C. The organic layer was washed with water (50 mL×3) and brine (50 mL×3), dried over anhydrous sodium sulfate, filtered and concentrated. The residue was purified via silica gel chromatography (petroleum ether:EtOAc=3:1) to provide the titled compound (700 mg, 38% yield) as a light yellow solid. ESI-MS (EI⁺, m/z): 1232.2 [M+Na]⁺. ¹HNMR (400 MHz, CDCl₃) δ 7.70-7.68 (m, 4H), 7.43-7.26 (m, 6H), 6.40-5.87 (m, 4H), 5.68-5.07 (m, 4H), 4.67 (s, 1H), 4.48-4.13 (m, 1H), 3.81-3.57 (m, 7H), 3.47-3.33 (m, 5H), 3.20-3.08 (m, 7H), 3.07-2.97 (m, 1H), 2.71-2.50 (m, 2H), 2.35-2.20 (m, 2H), 2.09-1.97 (m, 3H), 1.70-1.66 (m, 6H), 1.61-1.58 (m, 11H), 1.38-1.20 (m, 10H), 1.15-1.10 (m, 5H), 1.09-1.05 (m, 10H), 0.98-0.73 (m, 13H), 0.71-0.66 (m, 1H).

Step 2: (23E,25E,27E,28E,32R,33S,34R,35R,37S,39S,41S,42S,43R,44R,53R)-53-hydroxy-42-[(1R)-2-[(1S,3R,4R)-4-(2-hydroxyethoxy)-3-methoxy-cyclohexyl]-1-methyl-ethyl]-41,43,44-trimethoxy-32,33,34,35,45,46-hexamethyl-62,63-dioxa-54-azatricyclohexatriaconta-23,25,27(45),28(46)-tetraene-47,48,49,50,51-pentone. To a solution of (36E,38E,40E,41E,47R,48S,49R,50R,52S,54S,56S,57S,58R,59R,68R)-57-[(1R)-2-[(1S,3R,4R)-4-[2-[tert-butyl(diphenyl)silyl]oxyethoxy]-3-methoxy-cyclohexyl]-1-methyl-ethyl]-68-hydroxy-56,58,59-trimethoxy-47,48,49,50,60,61-hexamethyl-77,78-dioxa-70-azatricyclohexatriaconta-36,38,40(60),41(61)-tetraene-62,63,64,65,66-pentone (0.7 g, 0.578 mmol) in THF (7 mL) was added Py.HF (0.457 g, 5.78 mmol) at 0° C. The mixture was stirred at 30° C. for 3 h then quenched by adding saturated aqueous NaHCO₃ (20 mL) and extracted with EtOAc (30 mL) at 0° C. The organic layer was washed with water (20 mL) and brine (20 mL), dried over anhydrous sodium sulfate, filtered and concentrated. The residue was purified via silica gel chromatography (petroleum ether:acetone=3:1) to provide the titled compound (250 mg, 44% yield) as a light yellow solid. ESI-MS (EI⁺, m/z): 995.0 [M+Na]⁺. ¹HNMR (400 MHz, CDCl₃) δ 6.48-6.01 (m, 4H), 5.71-5.08 (m, 4H), 4.68 (s, 1H), 4.50-4.08 (m, 1H), 3.83-3.55 (m, 7H), 3.45-3.08 (m, 17H), 3.00-2.51 (m, 2H), 2.40-2.32 (m, 2H), 2.16-1.97 (m, 3H), 1.75-1.58 (m, 15H), 1.30-1.24 (m, 6H), 1.15-1.10 (m, 5H), 0.98-0.82 (m, 17H), 0.78-0.68 (m, 1H).

Step 3: (23E,25E,27E,28E,36R,37S,38R,39R,41S,43S,46S,47R,48R,57R)-57-hydroxy-46-[(1R)-2-[(1S,3R,4R)-4-(2-hydroxyethoxy)-3-methoxy-cyclohexyl]-1-methyl-ethyl]-47,48-dimethoxy-45-[2-(2-methoxyethoxy)ethoxy]-36,37,38,39,49,50-hexamethyl-66,67-dioxa-58-azatricyclohexatriaconta-23,25,27(49),28(50)-tetraene-51,52,53,54,55-pentone (I-35). To a solution of (23E,25E,27E,28E,32R,33S,34R,35R,37S,39S,41S,42S,43R,44R,53R)-53-hydroxy-42-[(1R)-2-[(1S,3R,4R)-4-(2-hydroxyethoxy)-3-methoxy-cyclohexyl]-1-methyl-ethyl]-41,43,44-trimethoxy-32,33,34,35,45,46-hexamethyl-62,63-dioxa-54-azatricyclohexatriaconta-23,25,27(45),28(46)-tetraene-47,48,49,50,51-pentone (0.25 g, 0.257 mmol) and 2-(2-methoxyethoxy)ethanol (0.618 g, 5.14 mmol) in THF (4 mL) was added HND-8 (80 mg) at 0° C. The mixture was stirred at 50° C. for 4 h then quenched by adding saturated aqueous NaHCO₃ (20 mL) and was extracted with EtOAc (30 mL) at 0° C. The organic layer was washed with water (20 mL) and brine (20 mL), dried over anhydrous sodium sulfate, filtered and concentrated. The residue was purified by reverse phase (85% CH₃CN in water) to provide I-35 (0.12 g, 44% yield) as a white solid. ESI-MS (EI⁺, m/z): 1082.8 [M+Na]⁺. ¹HNMR (400 MHz, CDCl₃) δ 6.42-5.98 (m, 4H), 5.85-5.08 (m, 4H), 4.72-4.65 (m, 1H), 4.51-4.10 (m, 1H), 3.83-3.75 (m, 2H), 3.65-3.55 (m, 7H), 3.40-3.06 (m, 17H), 2.71-2.46 (m, 2H), 2.40-2.20 (m, 2H), 2.15-1.88 (m, 3H), 1.75-1.58 (m, 21H), 1.42-1.30 (m, 5H), 1.19-1.00 (m, 13H), 0.97-0.82 (m, 10H), 0.78-0.68 (m, 1H).

Step 4: (23E,25E,27E,28E,36R,37S,38R,39R,41S,43S,45S,46S,47R,48R,57R)-57-hydroxy-46-[(1R)-2-[(1S,3R,4R)-4-(2-hydroxyethoxy)-3-methoxy-cyclohexyl]-1-methyl-ethyl]-47,48-dimethoxy-45-[2-(2-methoxyethoxy)ethoxy]-36,37,38,39,49,50-hexamethyl-66,67-dioxa-58-azatricyclohexatriaconta-23,25,27(49),28(50)-tetraene-51,52,53,54,55-pentone (I-37) and (23E,25E,27E,28E,36R,37S,38R,39R,41S,43S,45R,46S,47R,48R,57R)-57-hydroxy-46-[(1R)-2-[(1S,3R,4R)-4-(2-hydroxyethoxy)-3-methoxy-cyclohexyl]-1-methyl-ethyl]-47,48-dimethoxy-45-[2-(2-methoxyethoxy)ethoxy]-36,37,38,39,49,50-hexamethyl-66,67-dioxa-58-azatricyclohexatriaconta-23,25,27(49),28(50)-tetraene-51,52,53,54,55-pentone (I-36). 140 mg of the epimeric mixture was purified via preparative chiral HPLC to provide I-37 (30 mg, 30% yield) as a white solid and I-36 (30 mg, 30% yield) as a white solid.

Chiral separation method:

Column: CHIRALPAK IC

Column size: 5.0 cm I.D.×25 cm L, 10 μm

Sample solution: 4 mg/mL in Mobile phase

Injection: 5 mL

Mobile phase: Hexane/EtOH=70/30(V/V)

Flow rate: 30 mL/min

Wave length: UV 254 nm

Temperature: 38° C.

I-37: ESI-MS (EI⁺, m/z): 1081.7 [M+Na]⁺. ¹H NMR (400 MHz, CDCl₃) δ 6.52-6.10 (m, 3H), 5.96 (dd, J=62.3, 11.6 Hz, 1H), 5.62 (ddd, J=40.8, 14.6, 7.8 Hz, 1H), 5.24 (ddd, J=66.7, 18.2, 10.9 Hz, 3H), 4.68 (s, 1H), 3.93-3.52 (m, 9H), 3.51-3.03 (m, 17H), 3.01-2.49 (m, 3H), 2.40-1.63 (m, 24H), 1.53-1.18 (m, 12H), 1.18-0.81 (m, 18H), 0.78-0.62 (m, 1H).
I-36: ESI-MS (EI⁺, m/z): 1081.7 [M+Na]⁺. ¹H NMR (400 MHz, CDCl₃) δ 6.56-5.81 (m, 4H), 5.75-5.15 (m, 4H), 4.01-3.51 (m, 16H), 3.51-3.06 (m, 20H), 2.85-2.49 (m, 2H), 2.45-1.64 (m, 18H), 1.47-1.19 (m, 10H), 1.17-0.61 (m, 19H).

### Example 20: Synthesis of (26E,28E,30E,31E,34R,35S,36R,37R,39S,41S,44S,46R,47R,56R)-44-[(1R)-2-[(1S,3R,4R)-3,4-dimethoxycyclohexyl]-1-methyl-ethyl]-43-(1,4-dioxan-2-ylmethoxy)-56-hydroxy-46,47-dimethoxy-34,35,36,37,48,49-hexamethyl-66,67-dioxa-57-azatricyclohexatriaconta-26,28,30(48),31(49)-tetraene-50,51,52,53,54-pentone (I-38), (26E,28E,30E,31E,34R,35S,36R,37R,39S,41S,43S,44S,46R,47R,56R)-44-[(1R)-2-[(1S,3R,4R)-3,4-dimethoxycyclohexyl]-1-methyl-ethyl]-43-(1,4-dioxan-2-ylmethoxy)-56-hydroxy-46,47-dimethoxy-34,35,36,37,48,49-hexamethyl-66,67-dioxa-57-azatricyclohexatriaconta-26,28,30(48),31(49)-tetraene-50,51,52,53,54-pentone (I-40), and (26E,28E,30E,31E,34R,35S,36R,37R,39S,41S,43R,44S,46R,47R,56R)-44-[(1R)-2-[(1S,3R,4R)-3,4-dimethoxycyclohexyl]-1-methyl-ethyl]-43-(1,4-dioxan-2-ylmethoxy)-56-hydroxy-46,47-dimethoxy-34,35,36,37,48,49-hexamethyl-66,67-dioxa-57-azatricyclohexatriaconta-26,28,30(48),31(49)-tetraene-50,51,52,53,54-pentone (I-39)

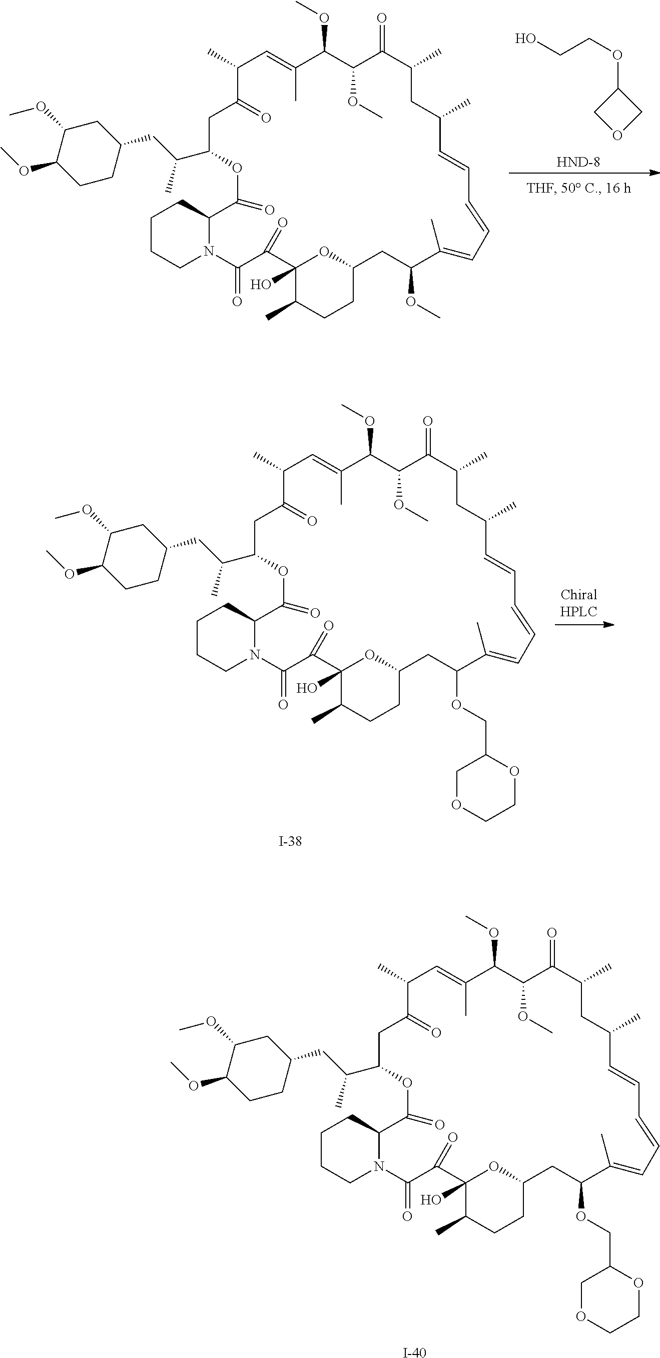

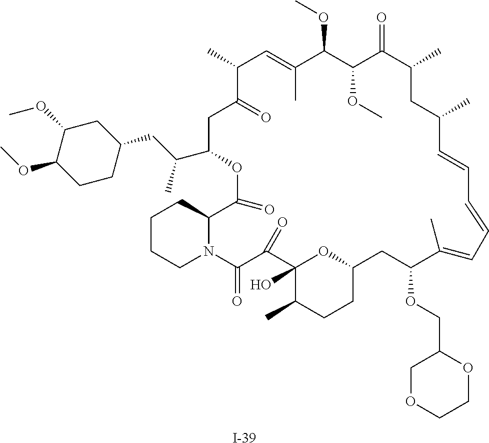

Step 1: (26E,28E,30E,31E,34R,35S,36R,37R,39S,41S,44S,46R,47R,56R)-44-[(1R)-2-[(1S,3R,4R)-3,4-dimethoxycyclohexyl]-1-methyl-ethyl]-43-(1,4-dioxan-2-ylmethoxy)-56-hydroxy-46,47-dimethoxy-34,35,36,37,48,49-hexamethyl-66,67-dioxa-57-azatricyclohexatriaconta-26,28,30(48),31(49)-tetraene-50,51,52,53,54-pentone (I-38). To a solution of (24E,26E,28E,29E,31R,32S,33R,34R,36S,38S,40S,41S,42R,43R,52R)-41-[(1R)-2-[(1S,3R,4R)-3,4-dimethoxycyclohexyl]-1-methyl-ethyl]-52-hydroxy-40,42,43-trimethoxy-31,32,33,34,44,45-hexamethyl-60,61-dioxa-53-azatricyclohexatriaconta-24,26,28(44),29(45)-tetraene-46,47,48,49,50-pentone (0.05 g, 0.053 mmol) and 2-(oxetan-3-yloxy)ethanol (0.125 g, 1.06 mmol) in THF (5 mL) was added HND-8 (0.02 g) at 50° C. under N₂. The reaction mixture was stirred for 16 hrs at 50° C. then cooled, filtered and concentrated. The residue was purified by reverse phase chromatography eluting with 80% CH₃CN in water to provide I-38 (0.019 g, 35% yield) as a white solid. ESI-MS (EI⁺, m/z): 1050.1 [M+Na]⁺. ¹HNMR (400 MHz, CDCl₃) δ 6.44-5.98 (m, 4H), 5.69-5.01 (m, 4H), 4.66-4.27 (m, 2H), 3.89-3.56 (m, 9H), 3.44-3.31 (m, 10H), 3.28-3.21 (m, 3H), 3.07-2.96 (m, 7H), 2.95-2.51 (m, 4H), 2.34-1.82 (m, 7H), 1.77-1.48 (m, 27H), 1.44-1.22 (m, 8H), 1.20-1.01 (m, 13H), 1.01-0.88 (m, 8H), 0.85-0.65 (m, 2H).

Step 2: (26E,28E,30E,31E,34R,35S,36R,37R,39S,41S,43S,44S,46R,47R,56R)-44-[(1R)-2-[(1S,3R,4R)-3,4-dimethoxycyclohexyl]-1-methyl-ethyl]-43-(1,4-dioxan-2-ylmethoxy)-56-hydroxy-46,47-dimethoxy-34,35,36,37,48,49-hexamethyl-66,67-dioxa-57-azatricyclohexatriaconta-26,28,30(48),31(49)-tetraene-50,51,52,53,54-pentone (I-40) and (26E,28E,30E,31E,34R,35S,36R,37R,39S,41S,43R,44S,46R,47R,56R)-44-[(1R)-2-[(1S,3R,4R)-3,4-dimethoxycyclohexyl]-1-methyl-ethyl]-43-(1,4-dioxan-2-ylmethoxy)-56-hydroxy-46,47-dimethoxy-34,35,36,37,48,49-hexamethyl-66,67-dioxa-57-azatricyclohexatriaconta-26,28,30(48),31(49)-tetraene-50,51,52,53,54-pentone (I-39). 140 mg of the epimeric mixture was purified via preparative chiral HPLC to provide I-40 (36.6 mg, 26% yield) as a white solid and I-39 (17.2 mg, 12% yield) as a white solid.

Chiral separation method:

Column: CHIRALPAK IC

Column size: 5.0 cm I.D.×25 cm L, 10 μm

Sample solution: 2 mg/mL in Mobile phase

Injection: 5 mL

Mobile phase: Hexane/EtOH=70/30(V/V)

Flow rate: 30 mL/min

Wave length: UV 254 nm

Temperature: 38° C.

I-40: ESI-MS (EI⁺, m/z): 1049.8 [M+Na]⁺. ¹H NMR (500 MHz, CDCl₃) δ 6.47-5.80 (m, 4H), 5.75-5.50 (m, 1H), 5.49-5.04 (m, 3H), 4.69-4.41 (m, 1H), 4.36-4.11 (m, 1H), 3.91-3.50 (m, 10H), 3.48-2.99 (m, 19H), 2.79-2.51 (m, 2H), 2.38-1.85 (m, 7H), 1.83-1.58 (m, 12H), 1.53-1.17 (m, 10H), 1.14-0.84 (m, 18H), 0.75 (d, J=10.9 Hz, 1H).
I-39: ESI-MS (EI⁺, m/z): 1049.8 [M+Na]⁺. ¹H NMR (500 MHz, CDCl₃) δ 6.60-5.70 (m, 4H), 5.66-5.01 (m, 4H), 4.72-4.14 (m, 2H), 4.10-3.50 (m, 9H), 3.49-2.98 (m, 18H), 2.59 (dd, J=79.6, 49.4 Hz, 3H), 2.40-1.64 (m, 19H), 1.52-1.20 (m, 10H), 1.19-0.65 (m, 20H).

### Example 21: Synthesis of (24E,26E,28E,29E,35R,36S,37R,38R,40S,42S,45S,46R,47R,56R)-45-[(1R)-2-[(1S,3R,4R)-3,4-dimethoxycyclohexyl]-1-methyl-ethyl]-56-hydroxy-46,47-dimethoxy-44-[2-(2-methoxyethylsulfonyl)ethoxy]-35,36,37,38,48,49-hexamethyl-66,67-dioxa-57-azatricyclohexatriaconta-24,26,28(48),29(49)-tetraene-50,51,52,53,54-pentone (I-41), (24E,26E,28E,29E,35R,36S,37R,38R,40S,42S,44S,45S,46R,47R,56R)-45-[(1R)-2-[(1S,3R,4R)-3,4-dimethoxycyclohexyl]-1-methyl-ethyl]-56-hydroxy-46,47-dimethoxy-44-[2-(2-methoxyethylsulfonyl)ethoxy]-35,36,37,38,48,49-hexamethyl-66,67-dioxa-57-azatricyclohexatriaconta-24,26,28(48),29(49)-tetraene-50,51,52,53,54-pentone (I-43), and (24E,26E,28E,29E,35R,36S,37R,38R,40S,42S,44R,45S,46R,47R,56R)-45-[(1R)-2-[(1S,3R,4R)-3,4-dimethoxycyclohexyl]-1-methyl-ethyl]-56-hydroxy-46,47-dimethoxy-44-[2-(2-methoxyethylsulfonyl)ethoxy]-35,36,37,38,48,49-hexamethyl-66,67-dioxa-57-azatricyclohexatriaconta-24,26,28(48),29(49)-tetraene-50,51,52,53,54-pentone (I-42)

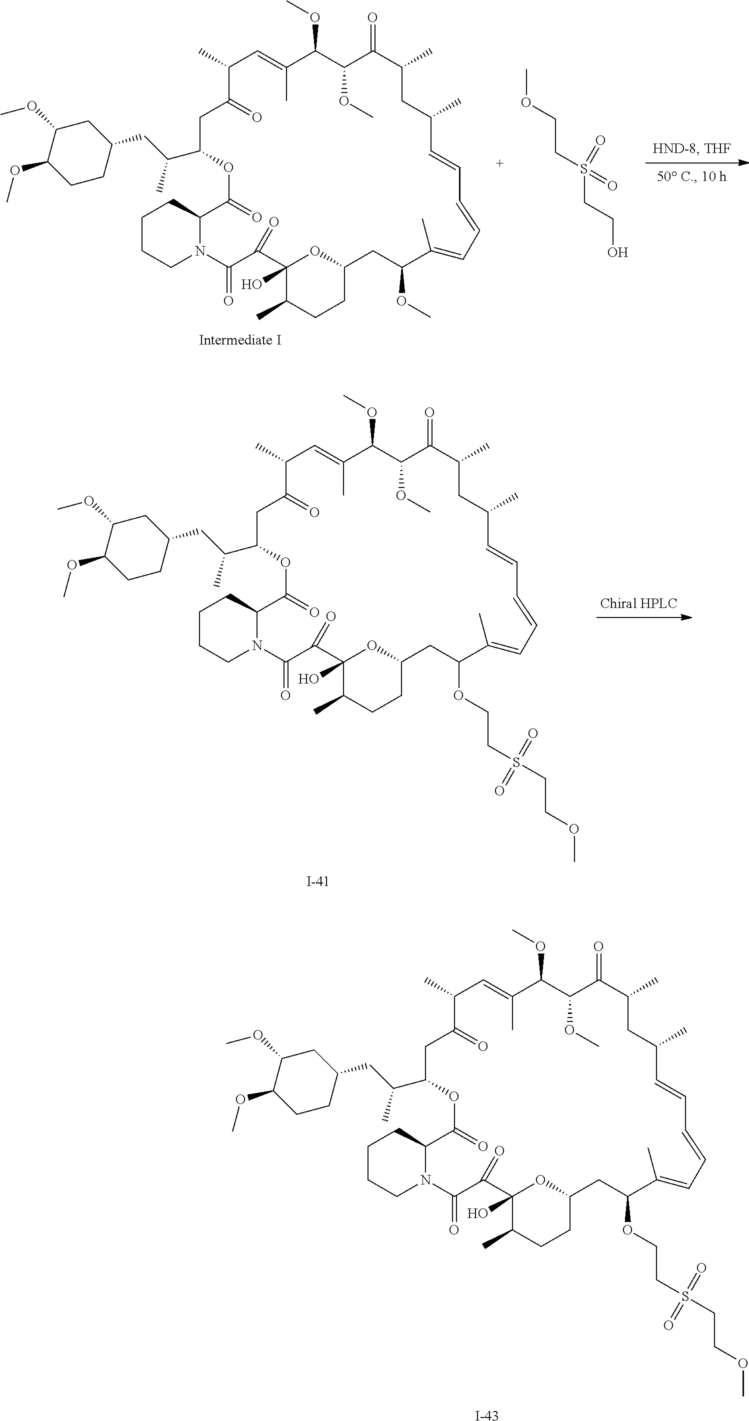

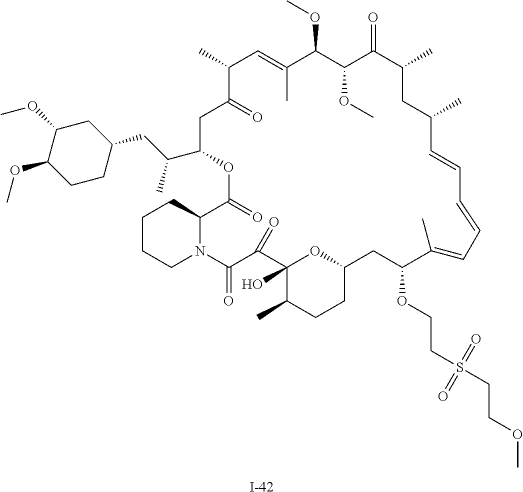

Step 1: (24E,26E,28E,29E,35R,36S,37R,38R,40S,42S,45S,46R,47R,56R)-45-[(1R)-2-[(1S,3R,4R)-3,4-dimethoxycyclohexyl]-1-methyl-ethyl]-56-hydroxy-46,47-dimethoxy-44-[2-(2-methoxyethylsulfonyl)ethoxy]-35,36,37,38,48,49-hexamethyl-66,67-dioxa-57-azatricyclohexatriaconta-24,26,28(48),29(49)-tetraene-50,51,52,53,54-pentone (I-41). To a solution of (24E,26E,28E,29E,31R,32S,33R,34R,36S,38S,40S,41S,42R,43R,52R)-41-[(1R)-2-[(1S,3R,4R)-3,4-dimethoxycyclohexyl]-1-methyl-ethyl]-52-hydroxy-40,42,43-trimethoxy-31,32,33,34,44,45-hexamethyl-60,61-dioxa-53-azatricyclohexatriaconta-24,26,28(44),29(45)-tetraene-46,47,48,49,50-pentone (Intermediate I, 0.15 g, 0.16 mmol) and 2-(2-methoxyethylsulfonyl)ethanol (0.268 g, 1.59 mmol) in THF (5 mL) was added HND-8 (50 mg) at 0° C. The mixture was stirred at 50° C. for 10 h then quenched by adding saturated aqueous NaHCO₃ solution (20 mL) and extracted with EtOAc (30 mL) at 0° C. The organic layer was washed with water (20 mL) and brine (20 mL), dried over anhydrous sodium sulfate, filtered and concentrated. The residue was purified via reverse phase chromatography (85% CH₃CN in water) to provide I-41 (44 mg, 26% yield) as a white solid. ESI-MS (EI⁺, m/z): 1100.0 [M+Na]⁺. ¹HNMR (400 MHz, CDCl₃) δ 6.50-5.96 (m, 4H), 5.80-5.02 (m, 4H), 4.83-4.75 (m, 1H), 4.76-4.39 (m, 1H), 3.85-3.80 (m, 2H), 3.75-3.53 (m, 4H), 3.45-3.10 (m, 17H), 3.09-2.85 (m, 3H), 2.81-2.48 (m, 3H), 2.35-1.85 (m, 7H), 1.76-1.57 (m, 21H), 1.39-1.22 (m, 5H), 1.17-0.83 (m, 18H), 0.79-0.66 (m, 1H).

Step 2: (24E,26E,28E,29E,35R,36S,37R,38R,40S,42S,44S,45S,46R,47R,56R)-45-[(1R)-2-[(1S,3R,4R)-3,4-dimethoxycyclohexyl]-1-methyl-ethyl]-56-hydroxy-46,47-dimethoxy-44-[2-(2-methoxyethylsulfonyl)ethoxy]-35,36,37,38,48,49-hexamethyl-66,67-dioxa-57-azatricyclohexatriaconta-24,26,28(48),29(49)-tetraene-50,51,52,53,54-pentone (I-43) and (24E,26E,28E,29E,35R,36S,37R,38R,40S,42S,44R,45S,46R,47R,56R)-45-[(1R)-2-[(1S,3R,4R)-3,4-dimethoxycyclohexyl]-1-methyl-ethyl]-56-hydroxy-46,47-dimethoxy-44-[2-(2-methoxyethylsulfonyl)ethoxy]-35,36,37,38,48,49-hexamethyl-66,67-dioxa-57-azatricyclohexatriaconta-24,26,28(48),29(49)-tetraene-50,51,52,53,54-pentone (I-42). 140 mg of the epimeric mixture was purified via preparative chiral HPLC to provide I-43 (18 mg, 20% yield) as a white solid and I-42 (26 mg, 29% yield) as a white solid.

Chiral separation method:

Column: CHIRALPAK IC

Column size: 5.0 cm I.D.×25 cm L, 10 μm

Sample solution: 2.5 mg/mL in Mobile phase

Injection: 8 mL

Mobile phase: Hexane/EtOH=50/50(V/V)

Flow rate: 40 mL/min

Wave length: UV 254 nm

Temperature: 35° C.

I-43: ESI-MS (EI⁺, m/z): 1099.7 [M+Na]⁺. ¹H NMR (400 MHz, CDCl₃) δ 6.48-5.83 (m, 4H), 5.56 (dd, J=14.8, 8.1 Hz, 1H), 5.49-5.02 (m, 3H), 4.75 (s, 1H), 3.91-3.51 (m, 9H), 3.46-3.18 (m, 18H), 3.16-2.98 (m, 6H), 2.96-2.45 (m, 3H), 2.38-1.66 (m, 17H), 1.54-1.16 (m, 13H), 1.25-0.65 (m, 19H).
I-42: ESI-MS (EI⁺, m/z): 1100.0 [M+Na]⁺. ¹H NMR (400 MHz, CDCl₃) δ 6.65-5.86 (m, 4H), 5.75-5.02 (m, 5H), 4.81-4.31 (m, 2H), 4.08-2.99 (m, 34H), 2.97-2.49 (m, 4H), 2.45-1.65 (m, 17H), 1.51-0.53 (m, 25H).

### Example 22: Synthesis of (23E,25E,27E,28E,35R,36S,37R,38R,40S,42S,44S,45S,47R,48R,57R)-47,57-dihydroxy-45-[(1R)-2-[(1S,3R,4R)-4-(3-hydroxypropoxy)-3-methoxy-cyclohexyl]-1-methyl-ethyl]-48-methoxy-35,36,37,38,49,50-hexamethyl-44-[2-(oxetan-3-yloxy)ethoxy]-68,69-dioxa-58-azatricyclohexatriaconta-23,25,27(49),28(50)-tetraene-51,52,53,54,55-pentone (I-45) and (23E,25E,27E,28E,35R,36S,37R,38R,40S,42S,44R,45S,47R,48R,57R)-47,57-dihydroxy-45-[(1R)-2-[(1S,3R,4R)-4-(3-hydroxypropoxy)-3-methoxy-cyclohexyl]-1-methyl-ethyl]-48-methoxy-35,36,37,38,49,50-hexamethyl-44-[2-(oxetan-3-yloxy)ethoxy]-68,69-dioxa-58-azatricyclohexatriaconta-23,25,27(49),28(50)-tetraene-51,52,53,54,55-pentone (I-44)

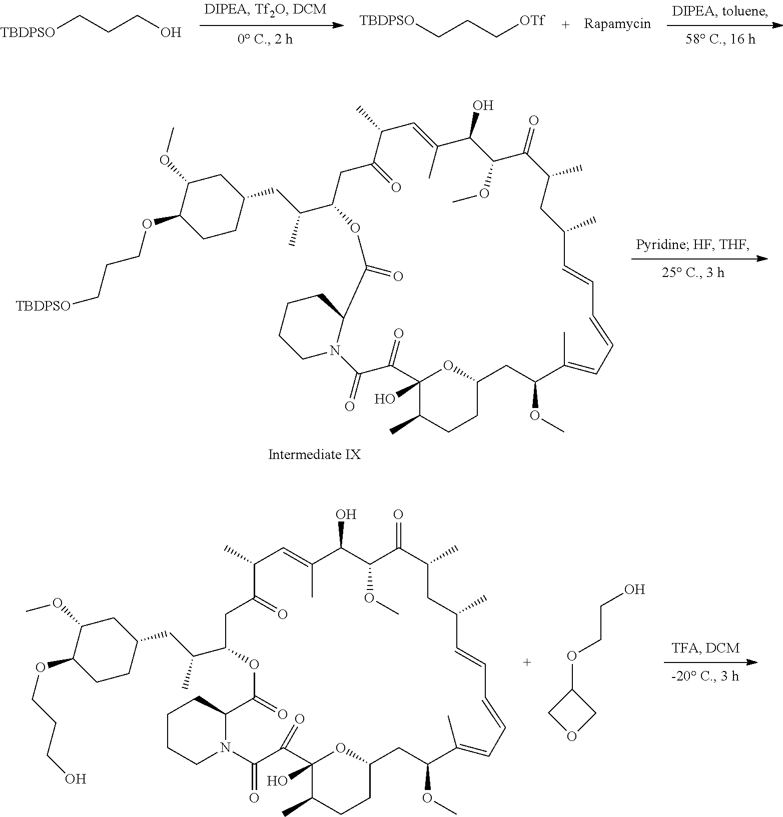

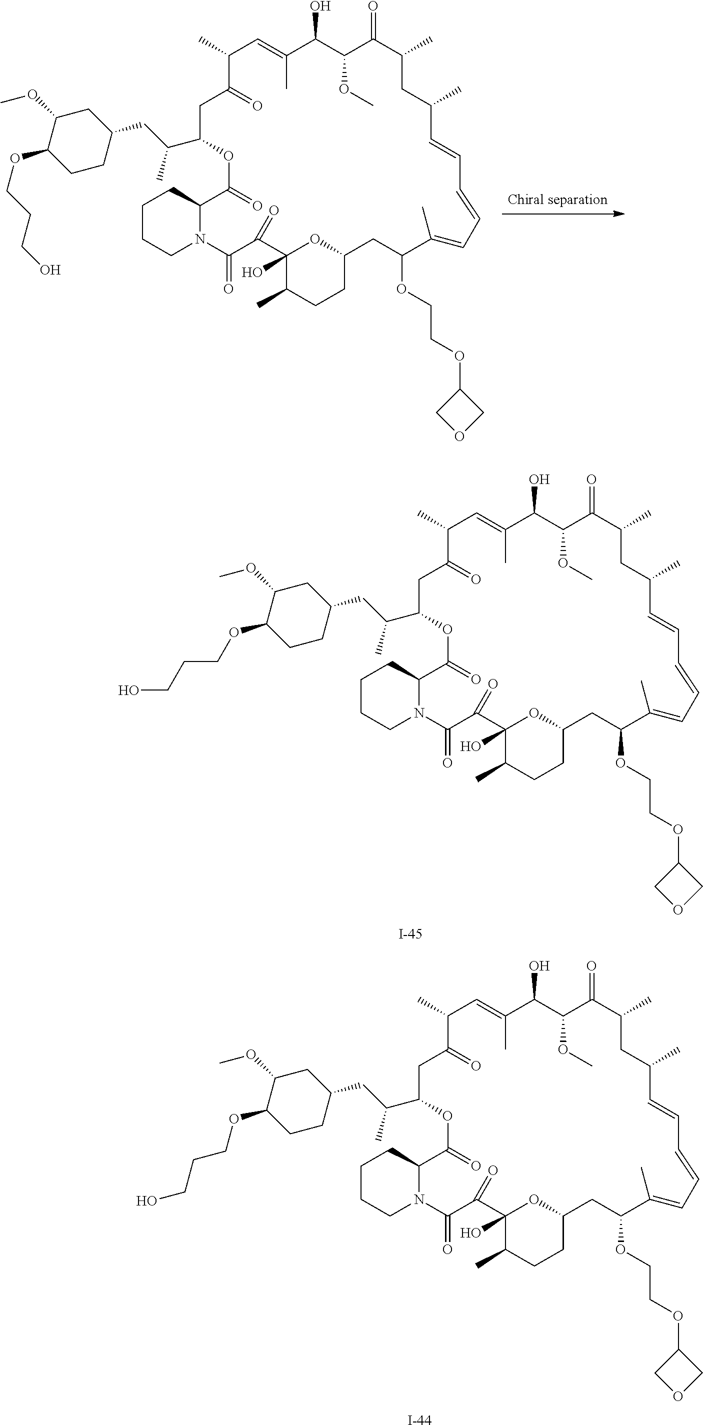

Step 1: 3-[tert-butyl (diphenyl) silyl] oxypropyl trifluoromethanesulfonate. To a mixture of 3-[tert-butyl(diphenyl)silyl]oxypropan-1-ol (7 g, 22.26 mmol) and DIPEA (5.82 mL, 33.39 mmol) in DCM (80 mL) at 0° C. under N2 was added trifluoromethylsulfonyl trifluoromethanesulfonate (6.91 g, 24.48 mmol) and the reaction stirred at 0° C. for 2 h. The mixture was diluted with DCM (150 mL), washed with saturated NaHCO₃(50 mL), water (50 mL) and brine (50 mL). The organic layer was dried over Na₂SO₄, filtrated and concentrated to afford 3-[tert-butyl(diphenyl)silyl]oxypropyl trifluoromethanesulfonate (9.9 g, 99.6% yield) as a brown oil. The curde was used in the next step without further purification. ¹H NMR (400 MHz, CDCl₃) δ 7.67-7.63 (m, 4H), 7.47-7.37 (m, 6H), 4.77-4.73 (t, J=6 Hz, 2H), 3.79-3.75 (t, J=6 Hz, 2H), 2.04-1.98 (m, 2H), 1.06 (s, 1H).

Step 2: (35E,37E,39E,40E,47R,48S,49R,50R,52S,54S,56S,57S,58R,59R,68R)-57-[(1R)-2-[(1S,3R,4R)-4-[3-[tert-butyl(diphenyl)silyl]oxypropoxy]-3-methoxy-cyclohexyl]-1-methyl-ethyl]-58,68-dihydroxy-56,59-dimethoxy-47,48,49,50,60,61-hexamethyl-78,79-dioxa-70-azatricyclohexatriaconta-35,37,39(60),40(61)-tetraene-62,63,64,65,66-pentone. A mixture of (22E,24E,26E,27E,29R,30S,31R,32R,34S,36S,38S,39S,40R,41R,50R)-40,50-dihydroxy-39-[(1R)-2-[(1S,3R,4R)-4-hydroxy-3-methoxy-cyclohexyl]-1-methyl-ethyl]-38,41-dimethoxy-29,30,31,32,42,43-hexamethyl-60,61-dioxa-51-azatricyclohexatriaconta-22,24,26(42),27(43)-tetraene-44,45,46,47,48-pentone (2 g, 2.19 mmol), 3-[tert-butyl(diphenyl) silyl] oxypropyl trifluoromethanesulfonate (9.77 g, 21.88 mmol) and N-ethyl-N-isopropyl-propan-2-amine (4.57 mL, 26.25 mmol) in toluene (40 mL) was stirred at 58° C. for 18 h. The mixture was poured into ice-cold saturated NaHCO₃ (150 mL), extracted with EtOAc (200 mL), and the organic layer was washed with water (150 mL×3) and brine (150 mL), dried over anhydrous Na₂SO₄, filtered and concentrated. The residue was purified via silica gel chromatography (petroleum ether:EA=3:1) to provide the titled compound (1.8 g, 68% yield) as a yellow solid. ESI-MS (EI⁺, m/z): 1232.7 [M+Na]⁺.

Step 3: (22E,24E,26E,27E,32R,33S,34R,35R,37S,39S,41S,42S,43R,44R,53R)-43,53-dihydroxy-42-[(1R)-2-[(1S,3R,4R)-4-(3-hydroxypropoxy)-3-methoxy-cyclohexyl]-1-methyl-ethyl]-41,44-dimethoxy-32,33,34,35,45,46-hexamethyl-63,64-dioxa-54-azatricyclohexatriaconta-22,24,26(45),27(46)-tetraene-47,48,49,50,51-pentone. To a solution of (35E,37E,39E,40E,47R,48S,49R,50R,52S,54S,56S,57S,58R,59R,68R)-57-[(1R)-2-[(1S,3R,4R)-4-[3-[tert-butyl(diphenyl)silyl]oxypropoxy]-3-methoxy-cyclohexyl]-1-methyl-ethyl]-58,68-dihydroxy-56,59-dimethoxy-47,48,49,50,60,61-hexamethyl-78,79-dioxa-70-azatricyclohexatriaconta-35,37,39(60),40(61)-tetraene-62,63,64,65,66-pentone (1.8 g, 1.49 mmol) in THF (15 mL) was added pyridine HF (1.2 mL, 14.87 mmol) and the reaction was stirred at 30° C. for 3 h. The mixture was quenched by adding saturated aqueous NaHCO₃ (20 mL) and extracted with EA (30 mL) at 0° C. The organic layer was washed with water (20 mL) and brine (20 mL), dried over anhydrous sodium sulfate, filtered and concentrated. The residue was purified via silica gel chromatography (petroleum ether:acetone=3:1) to obtain the titled compound (1.1 g, 76% yield) as a light yellow solid. ESI-MS (EI⁺, m/z): 994.7 [M+Na]⁺.

Step 4: (23E,25E,27E,28E,35R,36S,37R,38R,40S,42S,45S,47R,48R,57R)-47,57-dihydroxy-45-[(1R)-2-[(1S,3R,4R)-4-(3-hydroxypropoxy)-3-methoxy-cyclohexyl]-1-methyl-ethyl]-48-methoxy-35,36,37,38,49,50-hexamethyl-44-[2-(oxetan-3-yloxy)ethoxy]-68,69-dioxa-58-azatricyclohexatriaconta-23,25,27(49),28(50)-tetraene-51,52,53,54,55-pentone. To a solution of (22E,24E,26E,27E,32R,33S,34R,35R,37S,39S,41S,42S,43R,44R,53R)-43,53-dihydroxy-42-[(1R)-2-[(1S,3R,4R)-4-(3-hydroxypropoxy)-3-methoxy-cyclohexyl]-1-methyl-ethyl]-41,44-dimethoxy-32,33,34,35,45,46-hexamethyl-63,64-dioxa-54-azatricyclohexatriaconta-22,24,26(45),27(46)-tetraene-47,48,49,50,51-pentone (0.2 g, 0.206 mmol) in DCM (4 mL) under nitrogen was added TFA (0.32 mL, 4.11 mmol) at −40° C., then 2-(oxetan-3-yloxy)ethanol (0.49 g, 4.11 mmol) was added. The reaction was stirred at −40° C. for 3 h then poured into ice cold saturated aqueous NaHCO₃ solution and extracted with DCM, dried, filtered and concentrated. The residue was then purified via reverse phase chromatography eluting with 80% CH₃CN in water to provide the titled compound (30 mg, 14% yield) as a white solid. ESI-MS (EI⁺, m/z): 1080.3 [M+Na]⁺. ¹H NMR (400 MHz, CDCl₃) δ 6.41-5.92 (m, 4H), 5.57-5.08 (m, 4H), 4.70-4.55 (m, 5H), 4.35-4.0 (m, 3H), 3.92-3.69 (m, 5H), 3.68-3.54 (m, 3H), 3.53-3.30 (m, 7H), 3.29-2.98 (m, 4H), 2.88-2.40 (m, 4H), 2.38-2.25 (m, 2H), 2.22-1.90 (m, 5H), 1.87-1.57 (m, 17H), 1.54-1.16 (m, 10H), 1.15-0.83 (m, 17H), 0.76-0.62 (m, 1H).

Step 5: (23E,25E,27E,28E,35R,36S,37R,38R,40S,42S,44S,45S,47R,48R,57R)-47,57-dihydroxy-45-[(1R)-2-[(1S,3R,4R)-4-(3-hydroxypropoxy)-3-methoxy-cyclohexyl]-1-methyl-ethyl]-48-methoxy-35,36,37,38,49,50-hexamethyl-44-[2-(oxetan-3-yloxy)ethoxy]-68,69-dioxa-58-azatricyclohexatriaconta-23,25,27(49),28(50)-tetraene-51,52,53,54,55-pentone (I-45) and (23E,25E,27E,28E,35R,36S,37R,38R,40S,42S,44R,45S,47R,48R,57R)-47,57-dihydroxy-45-[(1R)-2-[(1S,3R,4R)-4-(3-hydroxypropoxy)-3-methoxy-cyclohexyl]-1-methyl-ethyl]-48-methoxy-35,36,37,38,49,50-hexamethyl-44-[2-(oxetan-3-yloxy)ethoxy]-68,69-dioxa-58-azatricyclohexatriaconta-23,25,27(49),28(50)-tetraene-51,52,53,54,55-pentone (I-44). 139 mg of (23E,25E,27E,28E,35R,36S,37R,38R,40S,42S,45S,47R,48R,57R)-47,57-dihydroxy-45-[(1R)-2-[(1S,3R,4R)-4-(3-hydroxypropoxy)-3-methoxy-cyclohexyl]-1-methyl-ethyl]-48-methoxy-35,36,37,38,49,50-hexamethyl-44-[2-(oxetan-3-yloxy)ethoxy]-68,69-dioxa-58-azatricyclohexatriaconta-23,25,27(49),28(50)-tetraene-51,52,53,54,55-pentone separated via chiral preparative HPLC then purified via silica gel chromatography (13% MeOH in petroleum ether:DCM:EA=3:3:1) to provide I-45 (30 mg, 22% yield) as a white solid and I-44 (17 mg, 12% yield) as a white solid.

Chiral separation method:

Column: CHIRALPAK IC

Column size: 2.5 cm I.D.×25 cm L, 10 μm

Sample solution: 2 mg/mL in mobile phase

Injection: 8 mL

Mobile phase: Hexane/EtOH=50/50(V/V)

Flow rate: 23 mL/min

Wave length: UV 254 nm

Temperature: 35° C.

I-45: ESI-MS (EI⁺, m/z): 1079.9 [M+Na]⁺. ¹H NMR (400 MHz, CDCl₃) δ 6.46-5.83 (m, 4H), 5.62-5.02 (m, 4H), 4.87-4.51 (m, 6H), 4.17 (d, J=5.0 Hz, 1H), 3.94-2.96 (m, 24H), 2.90-2.52 (m, 3H), 2.41-1.71 (m, 15H), 1.62-1.40 (m, 8H), 1.39-1.18 (m, 7H), 1.15-0.79 (m, 18H), 0.76-0.65 (m, 1H).

I-44: ESI-MS (EI⁺, m/z): 1079.8 [M+Na]⁺. ¹H NMR (400 MHz, CDCl₃) δ 6.48-5.81 (m, 4H), 5.75-5.08 (m, 4H), 4.87-4.53 (m, 5H), 4.40-4.11 (m, 2H), 4.06-3.71 (m, 5H), 3.70-2.89 (m, 24H), 2.87-1.74 (m, 17H), 1.55-1.17 (m, 11H), 1.16-0.82 (m, 18H), 0.73-0.65 (m, 1H).

### Example 23: Synthesis of (24E,26E,28E,29E,37R,38S,39R,40R,42S,44S,47S,48R,49R,58R)-58-hydroxy-48,49-dimethoxy-46-[2-(2-methoxyethoxy)ethoxy]-47-[(1R)-2-[(1S,3R,4R)-3-methoxy-4-(2-methoxyethoxy)cyclohexyl]-1-methyl-ethyl]-37,38,39,40,50,51-hexamethyl-66,67-dioxa-59-azatricyclohexatriaconta-24,26,28(50),29(51)-tetraene-52,53,54,55,56-pentone (I-46) and (24E,26E,28E,29E,37R,38S,39R,40R,42S,44S,46S,47S,48R,49R,58R)-58-hydroxy-48,49-dimethoxy-46-[2-(2-methoxyethoxy)ethoxy]-47-[(1R)-2-[(1S,3R,4R)-3-methoxy-4-(2-methoxyethoxy)cyclohexyl]-1-methyl-ethyl]-37,38,39,40,50,51-hexamethyl-66,67-dioxa-59-azatricyclohexatriaconta-24,26,28(50),29(51)-tetraene-52,53,54,55,56-pentone (I-47)

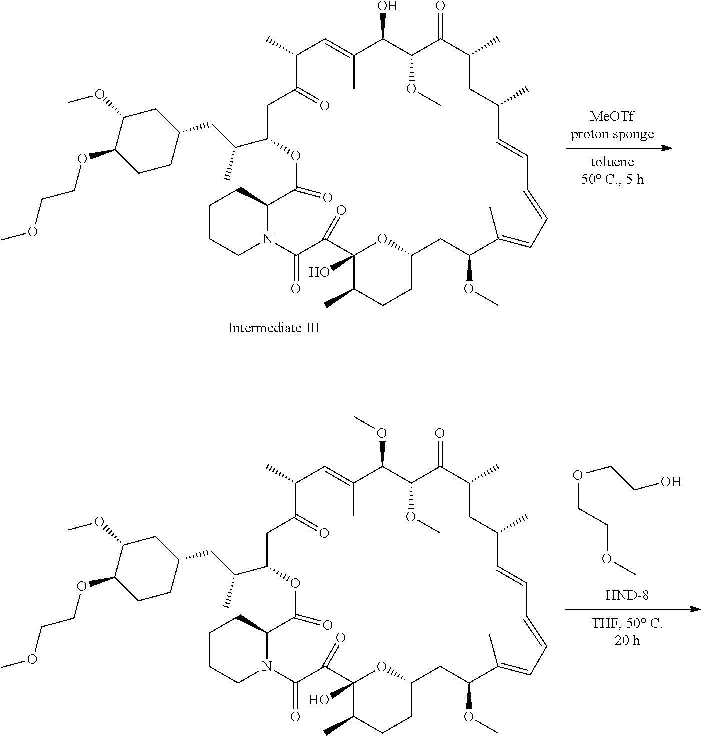

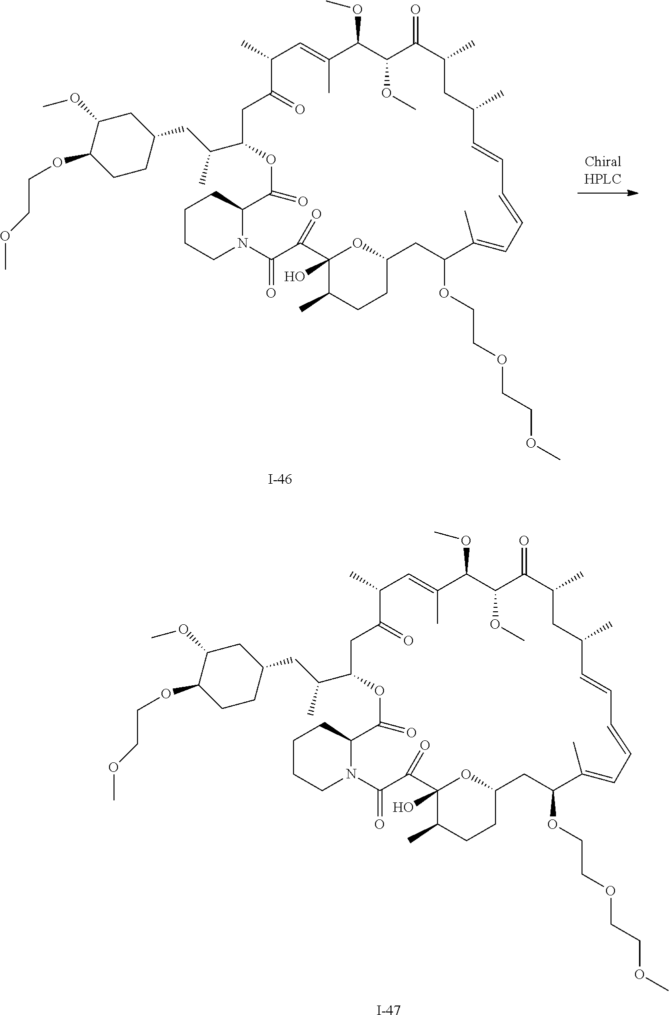

Step 1:(24E,26E,28E,29E,31R,32S,33R,34R,36S,38S,40S,41S,42R,43R,52R)-41-[(1R)-2-[(1S,3R,4R)-3,4-dimethoxycyclohexyl]-1-methyl-ethyl]-52-hydroxy-40,42,43-trimethoxy-31,32,33,34,44,45-hexamethyl-60,61-dioxa-53-azatricyclohexatriaconta-24,26,28(44),29(45)-tetraene-46,47,48,49,50-pentone. To a suspension of (23E,25E,27E,28E,32R,33S,34R,35R,37S,39S,41S,42S,43R,44R,53R)-43,53-dihydroxy-41,44-dimethoxy-42-[(1R)-2-[(1S,3R,4R)-3-methoxy-4-(2-methoxyethoxy)cyclohexyl]-1-methyl-ethyl]-32,33,34,35,45,46-hexamethyl-62,63-dioxa-54-azatricyclohexatriaconta-23,25,27(45),28(46)-tetraene-47,48,49,50,51-pentone (Intermediate III, 1.4 g, 1.44 mmol) and 1, 8-Bis(dimethylamino)naphtalene (4.63 g, 21.6 mmol) in toluene (24 mL) was added methyl trifluoromethanesulfonate (2.36 g, 14.4 mmol, 1.58 mL) dropwise at rt under N₂. After the addition, the mixture was heated to 50° C. for 3 hrs then filtered, diluted with EtOAc (60 mL), washed with saturated aqueous. NH₄Cl solution (60 mL×2), water (60 mL) and brine (60 mL). The organic layer was dried over anhydrous sodium sulfate, filtered and concentrated. The residue was purified via silica gel chromatography (petroleum ether:EtOAc=7: 3) and reverse phase chromatography eluting with 80% CH₃CN in water to provide the titled compound (0.22 g, 15% yield) as a white solid. ESI-MS (EI+, m/z): 1009.5 [M+Na]⁺.

Step 2: (24E,26E,28E,29E,37R,38S,39R,40R,42S,44S,47S,48R,49R,58R)-58-hydroxy-48,49-dimethoxy-46-[2-(2-methoxyethoxy)ethoxy]-47-[(1R)-2-[(1S,3R,4R)-3-methoxy-4-(2-methoxyethoxy)cyclohexyl]-1-methyl-ethyl]-37,38,39,40,50,51-hexamethyl-66,67-dioxa-59-azatricyclohexatriaconta-24,26,28(50),29(51)-tetraene-52,53,54,55,56-pentone (I-46). To a solution of (24E,26E,28E,29E,33R,34S,35R,36R,38S,40S,42S,43S,44R,45R,54R)-54-hydroxy-42,44,45-trimethoxy-43-[(1R)-2-[(1S,3R,4R)-3-methoxy-4-(2-methoxyethoxy)cyclohexyl]-1-methyl-ethyl]-33,34,35,36,46,47-hexamethyl-62,63-dioxa-55-azatricyclohexatriaconta-24,26,28(46),29(47)-tetraene-48,49,50,51,52-pentone (0.1 g, 0.101 mmol) and 2-(2-methoxyethoxy)ethanol (0.244 g, 2.03 mmol) in THF (10 mL) was added HND-8 (0.04 g) at 50° C. under N₂. The reaction mixture was stirred for 20 hrs at 50° C. then cooled, filtered and the filtrate poured into saturated aqueous NaHCO₃ solution (20 mL) at 0° C. and extracted with EtOAc (15 mL). The organic layer was washed with water (15 mL) and brine (15 mL), dried over anhydrous sodium sulfate, filtered and concentrated. The residue was purified via silica gel chromatography (EtOAc:petroleum ether=4:1) to provide I-46 (0.065 g, 60% yield) as a white solid. ESI-MS (EI⁺, m/z): 1095.8 [M+Na]⁺. ¹HNMR (400 MHz, CDCl₃) δ 6.43-5.84 (m, 4H), 5.72-5.06 (m, 4H), 4.84-4.17 (m, 2H), 3.96-3.73 (m, 4H), 3.70-3.52 (m, 10H), 3.50-3.43 (m, 4H), 3.41-3.30 (m, 8H), 3.29-3.20 (m, 3H), 3.18-2.99 (m, 5H), 2.96-2.50 (m, 4H), 2.35-2.14 (m, 3H), 2.05-1.84 (m, 5H), 1.80-1.56 (m, 21H), 1.55-1.23 (m, 10H), 1.16-1.00 (m, 11H), 0.97-0.84 (m, 9H), 0.81-0.69 (m, 1H).

Step 3: (24E,26E,28E,29E,37R,38S,39R,40R,42S,44S,46S,47S,48R,49R,58R)-58-hydroxy-48,49-dimethoxy-46-[2-(2-methoxyethoxy)ethoxy]-47-[(1R)-2-[(1S,3R,4R)-3-methoxy-4-(2-methoxyethoxy)cyclohexyl]-1-methyl-ethyl]-37,38,39,40,50,51-hexamethyl-66,67-dioxa-59-azatricyclohexatriaconta-24,26,28(50),29(51)-tetraene-52,53,54,55,56-pentone (I-47). 50 mg of the epimeric mixture was purified via preparative chiral HPLC and then by silica gel chromatography (petroleum ether:DCM:EtOAc:MeOH=3:3:1:0.2) to provide I-47 (13 mg, 26% yield) as a white solid.

Chiral separation method:

Column: CHIRALPAK IC

Column size: 5.0 cm I.D.×25 cm L, 10 μm

Sample solution: 0.55 mg/mL in Mobile phase

Injection: 15 mL

Mobile phase: Hexane/EtOH=70/30(V/V)

Flow rate: 30 mL/min

Wave length: UV 254 nm

Temperature: 38° C.

ESI-MS (EI+, m/z): 1095.8 [M+Na]⁺. ¹H NMR (400 MHz, CDCl₃) δ 6.44-5.72 (m, 4H), 5.72-4.98 (m, 4H), 3.96-3.14 (m, 32H), 3.05 (d, J=7.9 Hz, 5H), 2.76-2.42 (m, 3H), 2.37-1.57 (m, 22H), 1.46-1.17 (m, 16H), 1.14-0.77 (m, 18H), 0.73-0.61 (m, 1H).

### Example 24: Synthesis of (23E,25E,27E,28E,34R,35S,36R,37R,39S,41S,43S,44S,45R,46R,55R)-55-hydroxy-44-[(1R)-2-[(1S,3R,4R)-4-hydroxy-3-methoxy-cyclohexyl]-1-methyl-ethyl]-45,46-dimethoxy-43-[2-(2-methoxyethoxy)ethoxy]-34,35,36,37,47,48-hexamethyl-64,65-dioxa-56-azatricyclohexatriaconta-23,25,27(47),28(48)-tetraene-49,50,51,52,53-pentone (I-49) and (23E,25E,27E,28E,34R,35S,36R,37R,39S,41S,43R,44S,45R,46R,55R)-55-hydroxy-44-[(1R)-2-[(1S,3R,4R)-4-hydroxy-3-methoxy-cyclohexyl]-1-methyl-ethyl]-45,46-dimethoxy-43-[2-(2-methoxyethoxy)ethoxy]-34,35,36,37,47,48-hexamethyl-64,65-dioxa-56-azatricyclohexatriaconta-23,25,27(47),28(48)-tetraene-49,50,51,52,53-pentone (I-48)

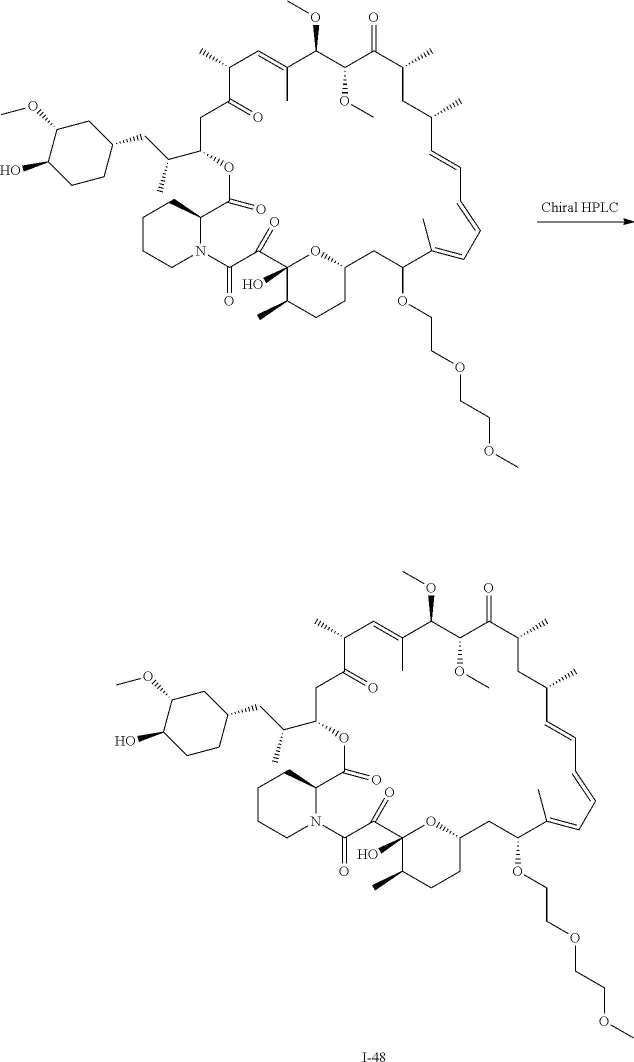

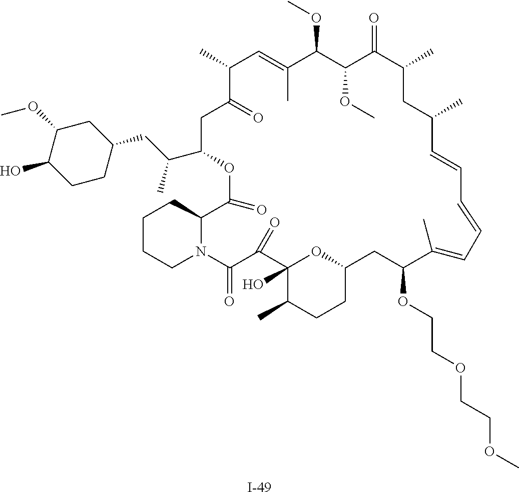

Step 1: (23E,25E,27E,28E,34R,35S,36R,37R,39S,41S,43S,44S,45R,46R,55R)-55-hydroxy-44-[(1R)-2-[(1S,3R,4R)-4-hydroxy-3-methoxy-cyclohexyl]-1-methyl-ethyl]-45,46-dimethoxy-43-[2-(2-methoxyethoxy)ethoxy]-34,35,36,37,47,48-hexamethyl-64,65-dioxa-56-azatricyclohexatriaconta-23,25,27(47),28(48)-tetraene-49,50,51,52,53-pentone (I-49) and (23E,25E,27E,28E,34R,35S,36R,37R,39S,41S,43R,44S,45R,46R,55R)-55-hydroxy-44-[(1R)-2-[(1S,3R,4R)-4-hydroxy-3-methoxy-cyclohexyl]-1-methyl-ethyl]-45,46-dimethoxy-43-[2-(2-methoxyethoxy)ethoxy]-34,35,36,37,47,48-hexamethyl-64,65-dioxa-56-azatricyclohexatriaconta-23,25,27(47),28(48)-tetraene-49,50,51,52,53-pentone (I-48). 116 mg of the epimeric mixture was separated via chiral preparative HPLC then purified via silica gel chromatography (hexane:DCM:EtOAc:MeOH=3:3:1:0.4) to provide I-49 (40 mg, 34% yield) as a white solid and I-48 (35 mg, 30% yield) as a white solid.

Chiral separation method:

Column: CHIRALPAK IC

Column size: 5.0 cm I.D.×25 cm L, 10 μm

Sample solution: 0.7 mg/mL in Mobile phase

Injection: 18 mL

Mobile phase: Hexane/EtOH=60/40(V/V)

Flow rate: 60 mL/min

Wave length: UV 254 nm

Temperature: 35° C.

I-49: ESI-MS (EI⁺, m/z): 1038.1 [M+Na]⁺. ¹H NMR (400 MHz, CDCl₃) δ 6.46-5.81 (m, 4H), 5.75-5.02 (m, 4H), 4.61 (d, J=16.7 Hz, 1H), 3.99-3.21 (m, 25H), 3.21-3.06 (m, 3H), 3.01-2.50 (m, 5H), 2.41-1.68 (m, 14H), 1.63-1.19 (m, 14H), 1.17-0.82 (m, 18H), 0.77-0.64 (m, 1H).
I-48: ESI-MS (EI⁺, m/z): 1038.1 [M+Na]⁺. ¹H NMR (400 MHz, CDCl₃) δ 6.62-5.87 (m, 4H), 5.77-5.02 (m, 4H), 4.72-4.27 (m, 1H), 3.99-3.06 (m, 28H), 3.00-2.47 (m, 6H), 2.43-1.70 (m, 15H), 1.52-1.20 (m, 12H), 1.18-0.79 (m, 18H), 0.69 (d, J=11.7 Hz, 1H).

### Example 25: (25E,27E,29E,30E,34R,35S,36R,37R,39S,41S,44S,46R,47R,56R)-44-[(1R)-2-[(1S,3R,4R)-3,4-dimethoxycyclohexyl]-1-methyl-ethyl]-56-hydroxy-46,47-dimethoxy-34,35,36,37,48,49-hexamethyl-43-[2-(oxetan-3-yloxy)ethoxy]-65,66-dioxa-57-azatricyclohexatriaconta-25,27,29(48),30(49)-tetraene-50,51,52,53,54-pentone (I-50)

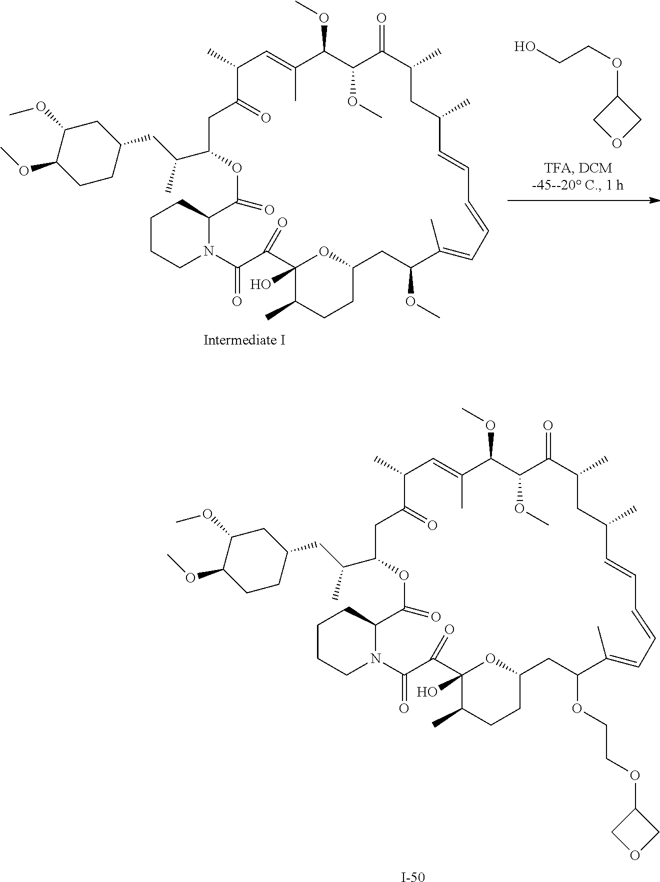

Step 1: (25E,27E,29E,30E,34R,35S,36R,37R,39S,41S,44S,46R,47R,56R)-44-[(1R)-2-[(1S,3R,4R)-3,4-dimethoxycyclohexyl]-1-methyl-ethyl]-56-hydroxy-46,47-dimethoxy-34,35,36,37,48,49-hexamethyl-43-[2-(oxetan-3-yloxy)ethoxy]-65,66-dioxa-57-azatricyclohexatriaconta-25,27,29(48),30(49)-tetraene-50,51,52,53,54-pentone (I-50). To a solution of (24E,26E,28E,29E,31R,32S,33R,34R,36S,38S,40S,41S,42R,43R,52R)-41-[(1R)-2-[(1S,3R,4R)-3,4-dimethoxycyclohexyl]-1-methyl-ethyl]-52-hydroxy-40,42,43-trimethoxy-31,32,33,34,44,45-hexamethyl-60,61-dioxa-53-azatricyclohexatriaconta-24,26,28(44),29(45)-tetraene-46,47,48,49,50-pentone (Intermediate I, 0.72 g, 0.76 mmol) in DCM (40 mL) was added 2,2,2-trifluoroacetic acid (1.18 mL, 15.28 mmol) dropwise at −55° C. under N₂. After addition, the reaction mixture was stirred for 10 min at −45° C. then 2-(oxetan-3-yloxy) ethanol (1.81 g, 15.28 mmol, dissolved in DCM) was added to the reaction mixture at the same temperature. The reaction mixture was stirred for 1 h at −45° C. then poured into saturated aqueous NaHCO₃ (60 mL) at 0° C. and extracted with DCM (60 mL). The organic layer was washed with water (60 mL) and brine (60 mL), dried over anhydrous sodium sulfate, filtered and the filtrate was concentrated in vacuo. The residue was purified via silica gel chromatography (100% EA) then by reverse-phase chromatography (eluting with 67% CH₃CN in water) to provide I-50 (0.07 g, 9% yield) as a white solid. ESI-MS (EI⁺, m/z): 1049.9 [M+Na]⁺. 1H NMR (400 MHz, CDCl₃) δ 6.41-6.01 (m, 4H), 5.35-4.94 (m, 4H), 4.78-4.57 (m, 5H), 4.50-4.13 (m, 1H), 3.89-3.58 (m, 4H), 3.55-3.31 (m, 11H), 3.28-3.201 (m, 4H), 3.21-3.10 (m, 3H), 3.07-2.97 (m, 2H), 2.78-2.54 (m, 3H), 2.30-2.27 (m, 2H), 2.10-1.95 (m, 5H), 1.79-1.48 (m, 13H), 1.45-1.04 (m, 19H), 0.97-0.84 (m, 8H) 0.78-0.73 (m, 1H).

### Example 26: Synthesis of (24E,26E,28E,29E,37R,38S,39R,40R,42S,44S,47S,48R,49R,58R)-47-[(1R)-2-[(1S,3R,4R)-3,4-dimethoxycyclohexyl]-1-methyl-ethyl]-58-hydroxy-48,49-dimethoxy-46-[2-[2-(2-methoxyethoxy)ethoxy]ethoxy]-37,38,39,40,50,51-hexamethyl-66,67-dioxa-59-azatricyclohexatriaconta-24,26,28(50),29(51)-tetraene-52,53,54,55,56-pentone (I-51)

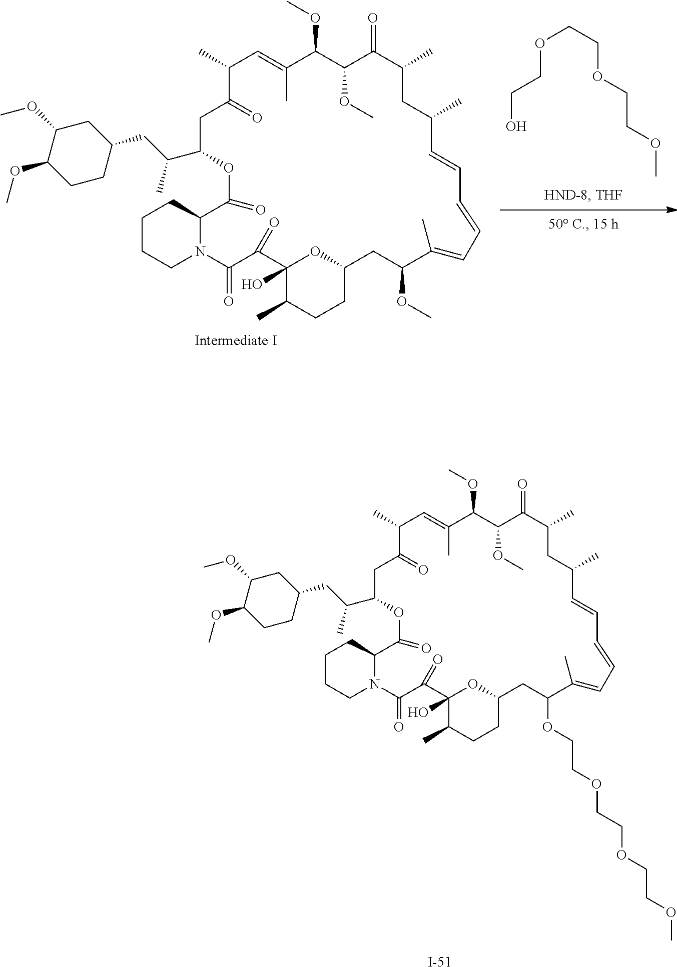

Step 1: (24E,26E,28E,29E,37R,38S,39R,40R,42S,44S,47S,48R,49R,58R)-47-[(1R)-2-[(1S,3R,4R)-3,4-dimethoxycyclohexyl]-1-methyl-ethyl]-58-hydroxy-48,49-dimethoxy-46-[2-[2-(2-methoxyethoxy)ethoxy]ethoxy]-37,38,39,40,50,51-hexamethyl-66,67-dioxa-59-azatricyclohexatriaconta-24,26,28(50),29(51)-tetraene-52,53,54,55,56-pentone (I-51). To a solution of (24E,26E,28E,29E,31R,32S,33R,34R,36S,38S,40S,41S,42R,43R,52R)-41-[(1R)-2-[(1S,3R,4R)-3,4-dimethoxycyclohexyl]-1-methyl-ethyl]-52-hydroxy-40,42,43-trimethoxy-31,32,33,34,44,45-hexamethyl-60,61-dioxa-53-azatricyclohexatriaconta-24,26,28(44),29(45)-tetraene-46,47,48,49,50-pentone (Intermediate I, 0.2 g, 0.212 mmol) and 2-[2-(2-methoxyethoxy)ethoxy]ethanol (0.349 g, 2.12 mmol) in THF (5 mL) was added HND-8 (50 mg) under N₂ at 50° C. The resulting solution was stirred for 15 h then diluted with EtOAc, filtered, then washed with water, brine, dried over Na₂SO₄, filtered again and concentrated. The residue was purified via silica gel chromatography (EtOAc:petroleum ether=1:0.8) and reverse phase chromatography (85% CH₃CN in water) to provide I-51 (40 mg, 18% yield) as a light yellow solid. ESI-MS (EI⁺, m/z): 1095.8 [M+Na]⁺. ¹HNMR (400 MHz, CDCl₃) δ 6.60-5.79 (m, 4H), 5.76-5.06 (m, 4H), 3.93-2.97 (m, 33H), 2.92-2.49 (m, 3H), 2.47-1.75 (m, 22H), 1.51-0.63 (m, 29H).

### Example 27: Synthesis of (25E,27E,29E,30E,35R,36S,37R,38R,40S,42S,45S,46R,47R,56R)-45-[(1R)-2-[(1S,3R,4R)-3,4-dimethoxycyclohexyl]-1-methyl-ethyl]-56-hydroxy-46,47-dimethoxy-35,36,37,38,48,49-hexamethyl-44-[3-(1,2,4-triazol-4-yl)propoxy]-67,68-dioxa-60-azatricyclohexatriaconta-25,27,29(48),30(49)-tetraene-50,51,52,53,54-pentone (I-52)

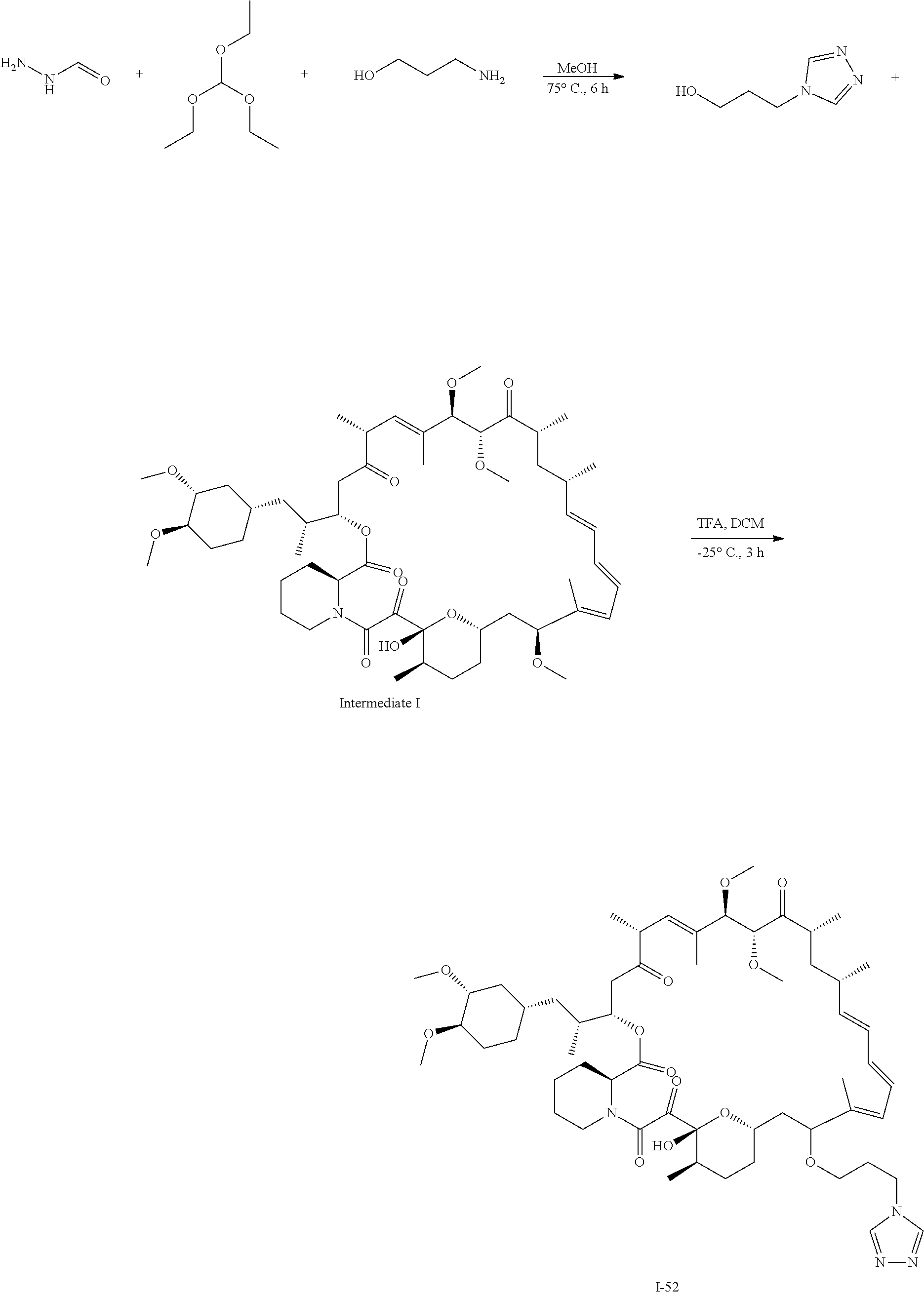

Step 1: 3-(1, 2, 4-triazol-4-yl) propan-1-ol. A mixture of formohydrazide (10 g, 166.51 mmol) and diethoxymethoxyethane (29.61 g, 199.82 mmol) in methanol (200 mL) was heated to reflux for 2 hrs, then 3-aminopropan-1-ol (12.51 g, 166.51 mmol) added dropwise. The reaction was refluxed for 4 hrs then concentrated and purified by reverse phase chromatography (10% CH₃CN in water) then by silica gel chromatography (DCM:CH₃OH=12:1) to afford the titled compound (20.6 g, 97% yield) as a pale solid. ESI-MS (EI+, m/z): 128.1 [M+H]⁺, T=0.189 min. ¹H NMR (400 MHz, MeOD-d₄) δ 8.49 (s, 2H), 4.18 (t, J=7.0 Hz, 2H), 3.48 (t, J=5.9 Hz, 2H), 2.00-1.90 (m, 2H).

Step 2 (25E,27E,29E,30E,35R,36S,37R,38R,40S,42S,45S,46R,47R,56R)-45-[(1R)-2-[(1S,3R,4R)-3,4-dimethoxycyclohexyl]-1-methyl-ethyl]-56-hydroxy-46,47-dimethoxy-35,36,37,38,48,49-hexamethyl-44-[3-(1,2,4-triazol-4-yl)propoxy]-67,68-dioxa-60-azatricyclohexatriaconta-25,27,29(48),30(49)-tetraene-50,51,52,53,54-pentone (I-52). To a mixture of 3-(1,2,4-triazol-4-yl)propan-1-ol (0.22 g, 1.75 mmol), (24E,26E,28E,29E,31R,32S,33R,34R,36S,38S,40S,41S,42R,43R,52R)-41-[(1R)-2-[(1S,3R,4R)-3,4-dimethoxycyclohexyl]-1-methyl-ethyl]-52-hydroxy-40,42,43-trimethoxy-31,32,33,34,44,45-hexamethyl-60,61-dioxa-53-azatricyclohexatriaconta-24,26,28(44),29(45)-tetraene-46,47,48,49,50-pentone (Intermediate I, 0.33 g, 0.35 mmol) and TFA (0.48 g, 4.20 mmol) in DCM (20 mL) was added 3-(1,2,4-triazol-4-yl)propan-1-ol (0.22 g, 1.75 mmol) and stirred at −30° C. for 3 hrs. The mixture was poured into saturated aqueous NaHCO₃ and the organic layer washed with water twice then brine. After concentration the residue was purified via reverse phase chromatography (MeOH:DCM=1:15) to provide I-52 (60 mg, 17% yield). ESI-MS (EI+, m/z): 1038.3 [M+H]⁺. ¹H NMR (400 MHz, CDCl₃) δ 8.36-7.82 (m, 1H), 6.49-5.92 (m, 4H), 5.75-4.96 (m, 5H), 4.51-3.92 (m, 2H), 3.64 (ddd, J=34.7, 33.2, 24.8 Hz, 4H), 3.48-3.20 (m, 11H), 3.08 (dd, J=38.8, 18.3 Hz, 7H), 2.92-2.42 (m, 5H), 2.25 (dd, J=76.9, 68.3 Hz, 8H), 1.94-1.46 (m, 19H), 1.44-0.96 (m, 20H), 0.96-0.62 (m, 9H).

### Example 28: Synthesis of (23E,25E,27E,28E,32R,33S,34R,35R,37S,39S,42S,44R,45R,55R)-42-[(1R)-2-[(1S,3R,4R)-4-(difluoromethoxy)-3-methoxy-cyclohexyl]-1-methyl-ethyl]-44,55-dihydroxy-45-methoxy-32,33,34,35,46,47-hexamethyl-41-[2-(oxetan-3-yloxy)ethoxy]-65,66-dioxa-56-azatricyclohexatriaconta-23,25,27(46),28(47)-tetraene-48,49,50,51,52-pentone (I-108), (23E,25E,27E,28E,32R,33S,34R,35R,37S,39S,41S,42S,44R,45R,55R)-42-[(1R)-2-[(1S,3R,4R)-4-(difluoromethoxy)-3-methoxy-cyclohexyl]-1-methyl-ethyl]-44,55-dihydroxy-45-methoxy-32,33,34,35,46,47-hexamethyl-41-[2-(oxetan-3-yloxy)ethoxy]-65,66-dioxa-56-azatricyclohexatriaconta-23,25,27(46),28(47)-tetraene-48,49,50,51,52-pentone (I-105) (23E,25E,27E,28E,32R,33S,34R,35R,37S,39S,41R,42S,44R,45R,55R)-42-[(1R)-2-[(1S,3R,4R)-4-(difluoromethoxy)-3-methoxy-cyclohexyl]-1-methyl-ethyl]-44,55-dihydroxy-45-methoxy-32,33,34,35,46,47-hexamethyl-41-[2-(oxetan-3-yloxy)ethoxy]-65,66-dioxa-56-azatricyclohexatriaconta-23,25,27(46),28(47)-tetraene-48,49,50,51,52-pentone (I-104)

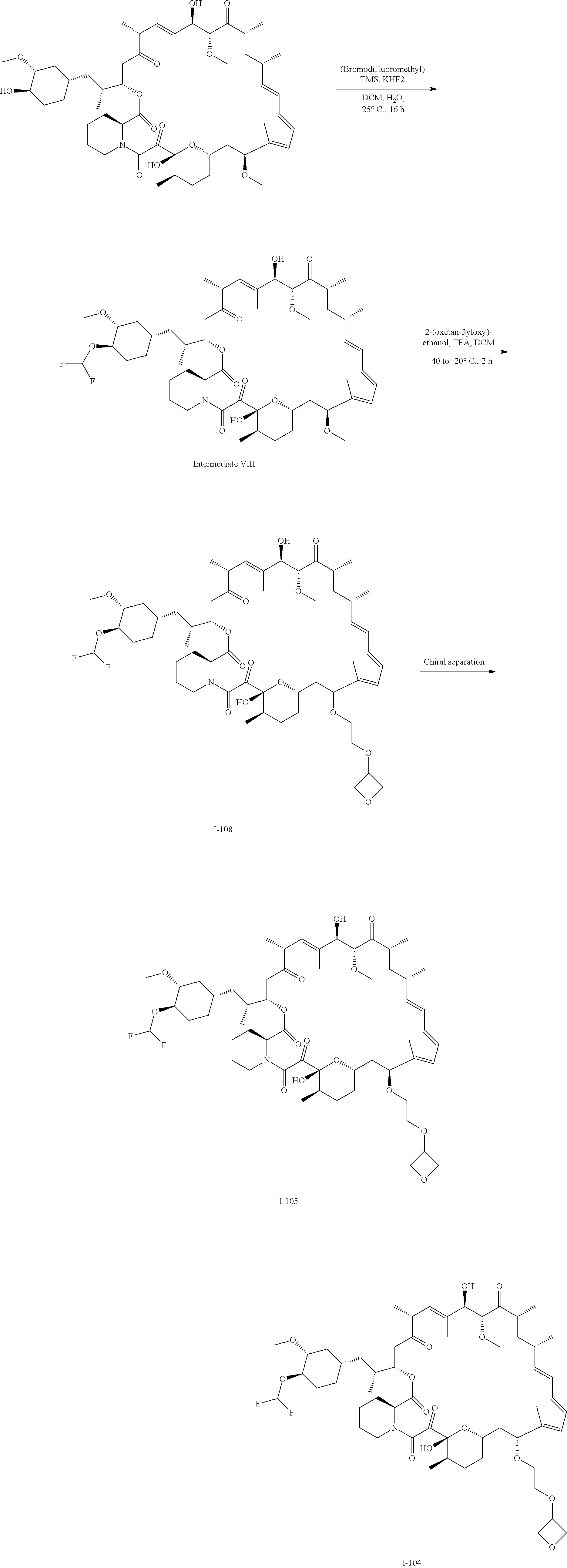

Step 1: (22E,24E,26E,27E,29R,30S,31R,32R,34S,36S,38S,39S,40R,41R,51R)-39-[(1R)-2-[(1S,3R,4R)-4-(difluoromethoxy)-3-methoxy-cyclohexyl]-1-methyl-ethyl]-40,51-dihydroxy-38,41-dimethoxy-29,30,31,32,42,43-hexamethyl-60,61-dioxa-52-azatricyclohexatriaconta-22,24,26(42),27(43)-tetraene-44,45,46,47,48-pentone. To a solution of rapamycin (2 g, 2.19 mmol) in DCM (30 mL) were added KHF₂ (2.56 g, 32.82 mmol in 2 mL water) and bromodifluoro(trimethylsilyl)methane (4.44 g, 21.88 mmol) at rt. The reaction mixture was stirred for 16 h then diluted with DCM and aqueous NaHCO₃, washed with water and brine, dried over Na₂SO₄, filtered and concentrated. The residue was purified via silica gel chromatography (PE:EA=3:1) to provide (22E,24E,26E,27E,29R,30S,31R,32R,34S,36S,38S,39S,40R,41R,51R)-39-[(1R)-2-[(1S,3R,4R)-4-(difluoromethoxy)-3-methoxy-cyclohexyl]-1-methyl-ethyl]-40,51-dihydroxy-38,41-dimethoxy-29,30,31,32,42,43-hexamethyl-60,61-dioxa-52-azatricyclohexatriaconta-22,24,26(42),27(43)-tetraene-44,45,46,47,48-pentone (0.4 g, 19% yield) as a white solid. ESI-MS (EI⁺, m/z): 986.5 [M+Na]⁺. ¹H NMR (400 MHz, CDCl₃) δ 6.43-5.86 (m, 4H), 5.58-5.07 (m, 4H), 4.49 (s, 1H), 4.18-4.09 (m, 2H), 3.89-3.56 (m, 4H), 3.47-3.28 (m, 7H), 3.19-3.02 (m, 4H), 2.90-2.55 (m, 3H), 2.41-2.21 (m, 2H), 2.20-1.91 (m, 6H), 1.90-1.41 (m, 20H), 1.40-1.13 (m, 7H), 1.12-0.81 (m, 14H), 0.80-0.67 (m, 1H).

Step 2: (23E,25E,27E,28E,32R,33S,34R,35R,37S,39S,42S,44R,45R,55R)-42-[(1R)-2-[(1S,3R,4R)-4-(difluoromethoxy)-3-methoxy-cyclohexyl]-1-methyl-ethyl]-44,55-dihydroxy-45-methoxy-32,33,34,35,46,47-hexamethyl-41-[2-(oxetan-3-yloxy)ethoxy]-65,66-dioxa-56-azatricyclohexatriaconta-23,25,27(46),28(47)-tetraene-48,49,50,51,52-pentone (I-108). To a solution of (22E,24E,26E,27E,29R,30S,31R,32R,34S,36S,38S,39S,40R,41R,51R)-39-[(1R)-2-[(1S,3R,4R)-4-(difluoromethoxy)-3-methoxy-cyclohexyl]-1-methyl-ethyl]-40,51-dihydroxy-38,41-dimethoxy-29,30,31,32,42,43-hexamethyl-60,61-dioxa-52-azatricyclohexatriaconta-22,24,26(42),27(43)-tetraene-44,45,46,47,48-pentone (0.3 g, 0.31 mmol) in DCM (6 mL) under nitrogen was added TFA (0.71 g, 6.22 mmol, 0.48 mL) at −40° C. The mixture was stirred for 10 minutes, then 2-(oxetan-3-yloxy)ethanol (0.74 g, 6.22 mmol) was added and the mixture stirred at −20° C. for 2h. The mixture was quenched by adding saturated aqueous NaHCO₃ (20 mL) and extracted with DCM (30 mL) at 0° C. The organic layer was washed with water (20 mL), brine (20 mL), dried over anhydrous sodium sulfate, filtered and concentrated in vacuo. The residue was purified by reverse phase column chromatography eluting with 80% CH₃CN in water to provide I-108 (50 mg, 15% yield) as a white solid. ESI-MS (EI⁺, m/z): 1072.7 [M+Na]⁺. ¹H NMR (400 MHz, CDCl₃) δ 6.41-5.93 (m, 4H), 5.57-5.07 (m, 4H), 5.82-4.53 (m, 5H), 4.31-3.99 (m, 2H), 3.93-3.65 (m, 3H), 3.63-3.04 (m, 13H), 2.90-2.27 (m, 5H), 2.26-1.86 (m, 5H), 1.85-1.55 (m, 17H), 1.53-1.17 (m, 9H), 1.16-0.77 (m, 17H), 0.76-0.65 (m, 1H).

Step 3: (23E,25E,27E,28E,32R,33S,34R,35R,37S,39S,41S,42S,44R,45R,55R)-42-[(1R)-2-[(1S,3R,4R)-4-(difluoromethoxy)-3-methoxy-cyclohexyl]-1-methyl-ethyl]-44,55-dihydroxy-45-methoxy-32,33,34,35,46,47-hexamethyl-41-[2-(oxetan-3-yloxy)ethoxy]-65,66-dioxa-56-azatricyclohexatriaconta-23,25,27(46),28(47)-tetraene-48,49,50,51,52-pentone (I-105) (23E,25E,27E,28E,32R,33S,34R,35R,37S,39S,41R,42S,44R,45R,55R)-42-[(1R)-2-[(1S,3R,4R)-4-(difluoromethoxy)-3-methoxy-cyclohexyl]-1-methyl-ethyl]-44,55-dihydroxy-45-methoxy-32,33,34,35,46,47-hexamethyl-41-[2-(oxetan-3-yloxy)ethoxy]-65,66-dioxa-56-azatricyclohexatriaconta-23,25,27(46),28(47)-tetraene-48,49,50,51,52-pentone (I-104). 0.17 g of I-108 was separated via chiral preparatory HPLC and then purified via silica gel chromatography (8% MeOH in a mixture of PE:DCM:EA=3:3:1) to provide I-105 (18 mg, 11% yield) as a white solid and I-104 (15 mg, 9% yield) as a white solid.

Chiral analysis method:

Column: CHIRALPAK IC (IC00CD-NA012)

Column size: 0.46 cm I.D.×15 cm L

Injection: 10 μl

Mobile phase: Hexane/EtOH=60/40(V/V)

Flow rate: 1.0 mL/min

Wavelength: UV 254 nm

Temperature: 35° C.

HPLC equipment: Shimadzu LC-20AT

I-105: ESI-MS (EI⁺, m/z): 1072.6 [M+Na]⁺. ¹H NMR (400 MHz, CDCl₃) δ 6.67-5.84 (m, 5H), 5.56-5.02 (m, 4H), 4.81-4.53 (m, 5H), 4.17 (d, J=5.6 Hz, 1H), 3.93-3.63 (m, 4H), 3.60-3.04 (m, 14H), 2.94-2.52 (m, 3H), 2.40-1.82 (m, 7H), 1.81-1.40 (m, 19H), 1.25 (ddd, J=23.7, 20.7, 10.9 Hz, 5H), 1.14-0.65 (m, 18H).
I-104: ESI-MS (EI⁺, m/z): 1072.7 [M+Na]⁺. ¹H NMR (400 MHz, CDCl₃) δ 6.63-5.90 (m, 5H), 5.73-5.03 (m, 4H), 4.80-4.54 (m, 5H), 4.31-3.66 (m, 5H), 3.59-3.04 (m, 14H), 2.93-1.96 (m, 10H), 1.94-1.59 (m, 12H), 1.54-1.19 (m, 11H), 1.15-0.63 (m, 19H).

### Example 29: Synthesis of (1R,2R,4S)-4-((2R)-2-((3S,6R,7E,9R,10R,12R,14S,15E,17E,19E,23S,26R,27R,34aS)-21-((1,4-dioxan-2-yl)methoxy)-9,27-dihydroxy-10-methoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)-2-methoxycyclohexyl (2-morpholinoethyl)carbamate (I-107), [(42S,44R,46R)-4-[(2R)-2-[(28E,30E,32E,33E,38R,39S,40R,41R,43S,45S,47S,48S,50R,51R,61R)-47-(1,4-dioxan-2-ylmethoxy)-50,61-dihydroxy-51-methoxy-38,39,40,41,52,53-hexamethyl-54,55,56,57,58-pentaoxo-76,77-dioxa-64-azatricyclohexatriaconta-28,30,32(52),33(53)-tetraen-48-yl]propyl]-46-methoxy-44-cyclohexyl] N-(2-morpholinoethyl)carbamate (I-103) and [(42S,44R,46R)-4-[(2R)-2-[(28E,30E,32E,33E,38R,39S,40R,41R,43S,45S,47R,48S,50R,51R,61R)-47-(1,4-dioxan-2-ylmethoxy)-50,61-dihydroxy-51-methoxy-38,39,40,41,52,53-hexamethyl-54,55,56,57,58-pentaoxo-76,77-dioxa-64-azatricyclohexatriaconta-28,30,32(52),33(53)-tetraen-48-yl]propyl]-46-methoxy-44-cyclohexyl] N-(2-morpholinoethyl)carbamate (I-102)

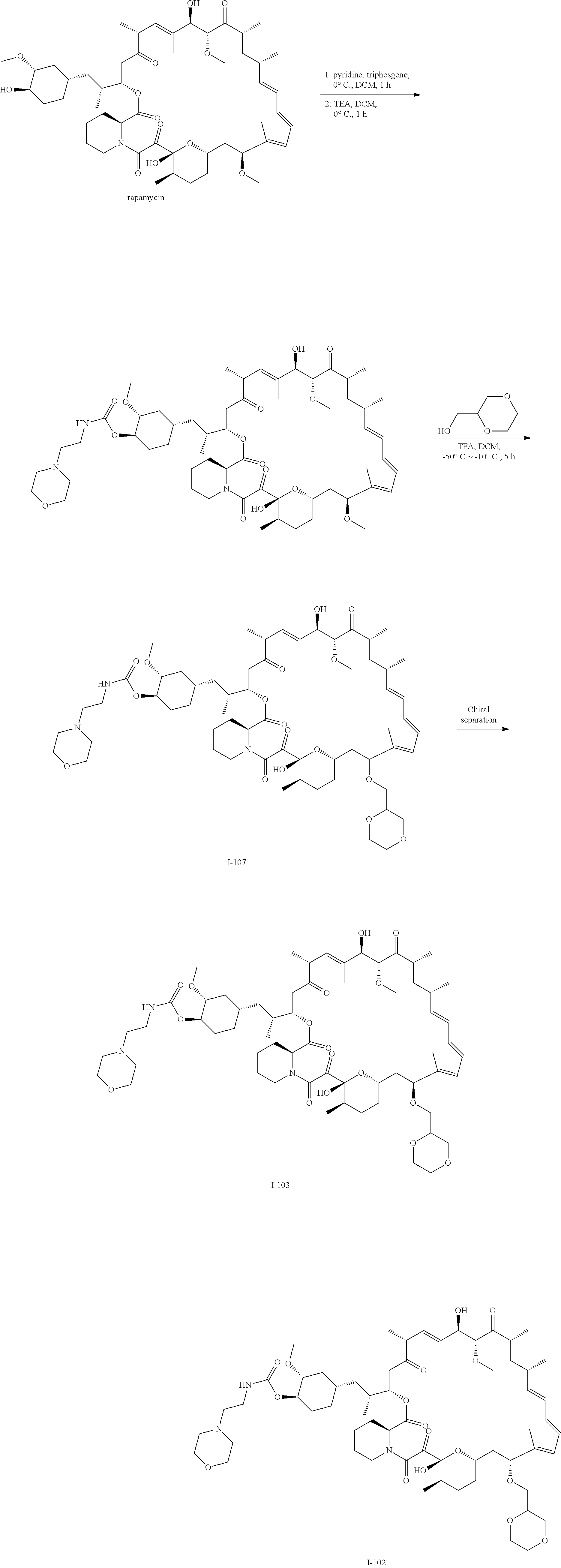

Step 1: (1R,2R,4S)-4-((R)-2-((3S,6R,7E,9R,10R,12R,14S,15E,17E,19E,21S,23S,26R,27R,34aS)-9,27-dihydroxy-10,21-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)-2-methoxycyclohexyl (2-morpholinoethyl)carbamate. To a solution of rapamycin (1 g, 1.09 mmol) and pyridine (0.35 mL, 4.38 mmol) in DCM (15 mL) was added triphosgene (0.325 g, 1.09 mmol) in DCM (0.5 mL) dropwise via syringe at 0° C. under argon. The reaction mixture was stirred for 1 h at 0° C. then TEA (1.22 mL, 8.75 mmol) and 2-morpholinoethanamine (2.85 mL, 21.88 mmol) were added to the mixture and the resulting solution was stirred at 0° C. for 1h then diluted with DCM, washed with NH₄Cl aqueous solution and water, brine, dried over Na₂SO₄, filtered and concentrated. The residue was purified via silica gel chromatography (8% MeOH in DCM) to provide (1R,2R,4S)-4-((R)-2-((3S,6R,7E,9R,10R,12R,14S,15E,17E,19E,21S,23S,26R,27R,34aS)-9,27-dihydroxy-10,21-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)-2-methoxycyclohexyl (2-morpholinoethyl)carbamate (0.25 g, 21% yield) as a light yellow solid. ESI-MS (EI⁺, m/z): 1070.4 [M+H]⁺.

Step 2: (1R,2R,4S)-4-((2R)-2-((3S,6R,7E,9R,10R,12R,14S,15E,17E,19E,23S,26R,27R,34aS)-21-((1,4-dioxan-2-yl)methoxy)-9,27-dihydroxy-10-methoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)-2-methoxycyclohexyl (2-morpholinoethyl)carbamate (I-107). To a solution of [(39S,41R,43R)-4-[(2R)-2-[(26E,28E,30E,31E,35R,36S,37R,38R,40S,42S,44S,45S,46R,47R,57R)-46,57-dihydroxy-44,47-dimethoxy-35,36,37,38,48,49-hexamethyl-50,51,52,53,54-pentaoxo-70,71-dioxa-60-azatricyclohexatriaconta-26,28,30(48),31(49)-tetraen-45-yl]propyl]-43-methoxy-41-cyclohexyl] N-(2-morpholinoethyl)carbamate (0.4 g, 0.37 mmol) in DCM (6 mL) was added TFA (1.15 mL, 14.95 mmol) at −50° C. The mixture was stirred for 10 minutes then 1, 4-dioxan-2-ylmethanol (1.32 g, 11.21 mmol) dissolved in DCM (10 mL) was added and the mixture was stirred at −10° C. for 5 h. The reaction was diluted with DCM and aqueous NaHCO₃, washed with water and brine, dried over Na₂SO₄, filtered and concentrated. The residue was purified via reverse-phase chromatography to provide I-107 (63 mg, 15% yield) as a white solid. ESI-MS (EI⁺, m/z): 1179.6 [M+Na]⁺. ¹H NMR (400 MHz, CDCl₃) δ 6.41-5.86 (m, 4H), 5.61-4.99 (m, 4H), 4.43 (dt, J=105.0, 47.9 Hz, 3H), 3.88-3.52 (m, 11H), 3.46-3.01 (m, 14H), 2.82-2.18 (m, 10H), 2.15-1.59 (m, 22H), 1.53-0.65 (m, 29H).

Step 3: [(42S,44R,46R)-4-[(2R)-2-[(28E,30E,32E,33E,38R,39S,40R,41R,43S,45S,47S,48S,50R,51R,61R)-47-(1,4-dioxan-2-ylmethoxy)-50,61-dihydroxy-51-methoxy-38,39,40,41,52,53-hexamethyl-54,55,56,57,58-pentaoxo-76,77-dioxa-64-azatricyclohexatriaconta-28,30,32(52),33(53)-tetraen-48-yl]propyl]-46-methoxy-44-cyclohexyl] N-(2-morpholinoethyl)carbamate (I-103) and [(42S,44R,46R)-4-[(2R)-2-[(28E,30E,32E,33E,38R,39S,40R,41R,43S,45S,47R,48S,50R,51R,61R)-47-(1,4-dioxan-2-ylmethoxy)-50,61-dihydroxy-51-methoxy-38,39,40,41,52,53-hexamethyl-54,55,56,57,58-pentaoxo-76,77-dioxa-64-azatricyclohexatriaconta-28,30,32(52),33(53)-tetraen-48-yl]propyl]-46-methoxy-44-cyclohexyl] N-(2-morpholinoethyl)carbamate (I-102). 124 mg of I-107 was separated via chiral preparatory HPLC to provide I-103 (23.7 mg, 19% yield) as a white solid and I-102 (21.3 mg, 17% yield) as a white solid.

Chiral analysis method:

Column: CHIRALPAK IC (IC00CD-NA012)

Column size: 0.46 cm I.D.×15 cm L

Injection: 20 μl

Mobile phase: Hexane/EtOH=50/50 (V/V)

Flow rate: 0.8 mL/min

Wavelength: UV 254 nm

Temperature: Room temperature

HPLC equipment: Shimadzu LC-20AD

I-103: ESI-MS (EI⁺, m/z): 1156.8 [M+H]⁺, 1178.8 [M+Na]⁺. 1H NMR (500 MHz, CDCl₃) δ 6.35-5.75 (m, 4H), 5.52-4.93 (m, 4H), 4.50 (s, 1H), 4.14 (dd, J=25.0, 13.4 Hz, 1H), 3.85-3.44 (m, 14H), 3.41-2.93 (m, 15H), 2.83-1.77 (m, 17H), 1.76-1.11 (m, 17H), 1.08-0.62 (m, 24H).
I-102: ESI-MS (EI⁺, m/z): 1156.8 [M+H]⁺, 1178.8 [M+Na]⁺. 1H NMR (500 MHz, CDCl₃) δ 6.58-5.85 (m, 4H), 5.57-5.08 (m, 4H), 4.63-3.92 (m, 3H), 3.88-3.04 (m, 24H), 2.80-1.94 (m, 14H), 1.87-1.44 (m, 28H), 1.13-0.60 (m, 20H).

### Example 30: Synthesis of (27E,29E,31E,32E,39R,40S,41R,44R,46S,48S,51S,53R,54R,63R)-51-[(1R)-2-[(1S,3R,4R)-4-[3-[(2S,6R)-2,6-dimethylmorpholin-4-yl]propoxy]-3-methoxy-cyclohexyl]-1-methyl-ethyl]-53,63-dihydroxy-54-methoxy-39,40,41,44,55,56-hexamethyl-50-[2-(oxetan-3-yloxy)ethoxy]-75,76-dioxa-64-azatricyclohexatriaconta-27,29,31(55),32(56)-tetraene-57,58,59,60,61-pentone (I-109), (27E,29E,31E,32E,39R,40S,41R,44R,46S,48S,50S,51S,53R,54R,63R)-51-[(1R)-2-[(1S,3R,4R)-4-[3-[(2S,6R)-2,6-dimethylmorpholin-4-yl]propoxy]-3-methoxy-cyclohexyl]-1-methyl-ethyl]-53,63-dihydroxy-54-methoxy-39,40,41,44,55,56-hexamethyl-50-[2-(oxetan-3-yloxy)ethoxy]-75,76-dioxa-64-azatricyclohexatriaconta-27,29,31(55),32(56)-tetraene-57,58,59,60,61-pentone I-101) and (27E,29E,31E,32E,39R,40S,41R,44R,46S,48S,50R,51S,53R,54R,63R)-51-[(1R)-2-[(1S,3R,4R)-4-[3-[(2S,6R)-2,6-dimethylmorpholin-4-yl]propoxy]-3-methoxy-cyclohexyl]-1-methyl-ethyl]-53,63-dihydroxy-54-methoxy-39,40,41,44,55,56-hexamethyl-50-[2-(oxetan-3-yloxy)ethoxy]-75,76-dioxa-64-azatricyclohexatriaconta-27,29,31(55),32(56)-tetraene-57,58,59,60,61-pentone (I-100)

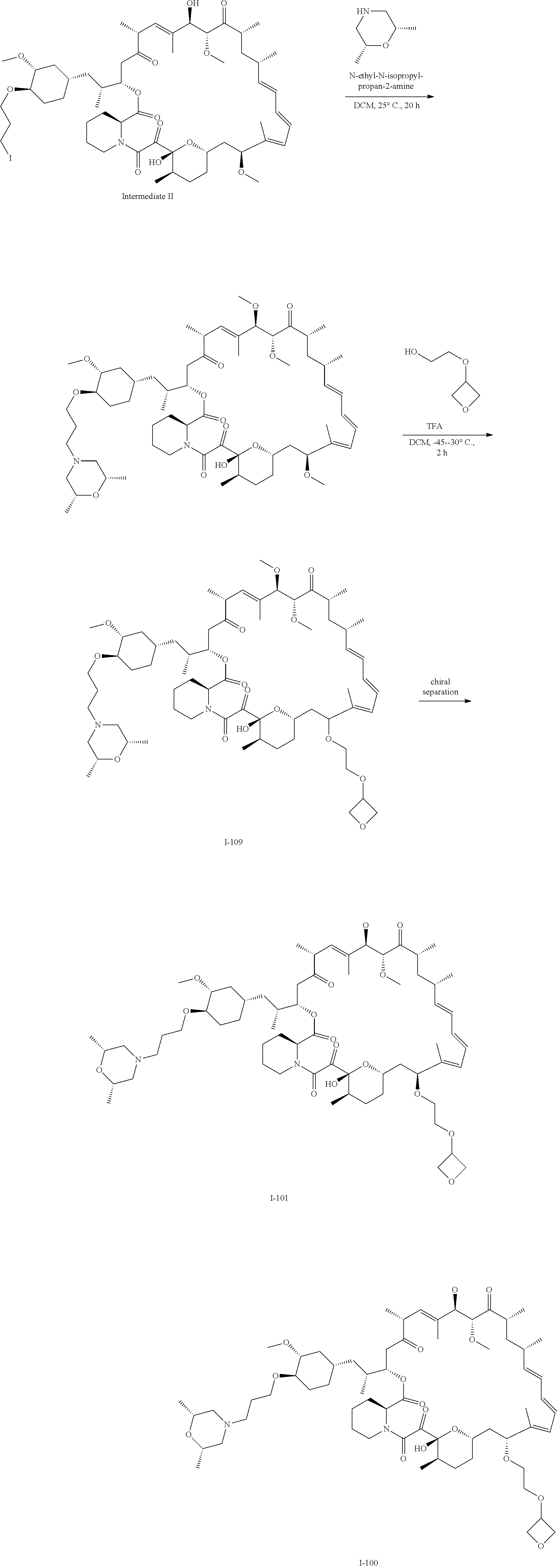

Step 1: (26E,28E,30E,31E,36R,37S,38R,41R,43S,45S,47S,48S,49R,50R,59R)-48-[(1R)-2-[(1S,3R,4R)-4-[3-[(2S,6R)-2,6-dimethylmorpholin-4-yl]propoxy]-3-methoxy-cyclohexyl]-1-methyl-ethyl]-49,59-dihydroxy-47,50-dimethoxy-36,37,38,41,51,52-hexamethyl-70,71-dioxa-60-azatricyclohexatriaconta-26,28,30(51),31(52)-tetraene-53,54,55,56,57-pentone. A solution of Intermediate II (0.5 g, 0.46 mmol) and N-ethyl-N-isopropyl-propan-2-amine (179.14 mg, 1.39 mmol, 0.24 mL) in DCM (10 mL) was stirred for 20 h at 25° C. The reaction mixture was diluted with DCM (30 mL) and washed with saturated NH₄Cl (30 mL×3) water (30 mL×3) and brine (30 mL×3), dried over anhydrous sodium sulfate, filtered and the concentrate. The residue was purified via reverse-phase chromatography eluting with 50% CH₃CN in water to provide (26E,28E,30E,31E,36R,37S,38R,41R,43S,45S,47S,48S,49R,50R,59R)-48-[(1R)-2-[(1S,3R,4R)-4-[3-[(2S,6R)-2,6-dimethylmorpholin-4-yl]propoxy]-3-methoxy-cyclohexyl]-1-methyl-ethyl]-49,59-dihydroxy-47,50-dimethoxy-36,37,38,41,51,52-hexamethyl-70,71-dioxa-60-azatricyclohexatriaconta-26,28,30(51),31(52)-tetraene-53,54,55,56,57-pentone (0.2 g, 40.5% yield) as a white solid. ESI-MS (EI⁺, m/z): 1069.1 [M+H]⁺, T=1.918 min, 98% purity, 254 n.

Step 2: (27E,29E,31E,32E,39R,40S,41R,44R,46S,48S,51S,53R,54R,63R)-51-[(1R)-2-[(1S,3R,4R)-4-[3-[(2S,6R)-2,6-dimethylmorpholin-4-yl]propoxy]-3-methoxy-cyclohexyl]-1-methyl-ethyl]-53,63-dihydroxy-54-methoxy-39,40,41,44,55,56-hexamethyl-50-[2-(oxetan-3-yloxy)ethoxy]-75,76-dioxa-64-azatricyclohexatriaconta-27,29,31(55),32(56)-tetraene-57,58,59,60,61-pentone (I-109). To a solution of (26E,28E,30E,31E,36R,37S,38R,41R,43S,45S,47S,48S,49R,50R,59R)-48-[(1R)-2-[(1S,3R,4R)-4-[3-[(2S,6R)-2,6-dimethylmorpholin-4-yl]propoxy]-3-methoxy-cyclohexyl]-1-methyl-ethyl]-49,59-dihydroxy-47,50-dimethoxy-36,37,38,41,51,52-hexamethyl-70,71-dioxa-60-azatricyclohexatriaconta-26,28,30(51),31(52)-tetraene-53,54,55,56,57-pentone (1.12 g, 1.05 mmol) in DCM (50 mL) was added 2,2,2-trifluoroacetic acid (3.58 g, 31.42 mmol, 2.42 mL) dropwise at −45° C. under N₂ and the reaction stirred for 10 minutes. 2-(oxetan-3-yloxy)ethanol (2.47 g, 20.95 mmol in DCM) was added and the mixture stirred for 2 h at −45° C. The reaction was poured into saturated NaHCO₃(5 mL) at 0° C. and extracted with DCM (10 mL). The organic layer was washed with water (10 mL) and brine (10 mL), dried over anhydrous sodium sulfate, filtered and the filtrate concentrated. The residue was purified via reverse-phase column eluting with 40% CH₃CN in water (0.001% HCOOH) to provide I-109 (0.074 g, 6% yield) as a white solid. ESI-MS (EI⁺, m/z): 1155.8 [M+H]⁺, T=1.849 min, 254 n. ¹H NMR (400 MHz, CDCl₃) δ 6.40-5.94 (m, 4H), 5.55-5.13 (m, 5H), 4.79-4.54 (m, 5H), 4.45-4.03 (m, 4H), 3.89-3.58 (m, 4H), 3.15-3.54 (m, 15H), 3.14-2.91 (m, 5H), 2.86-2.39 (m, 3H), 2.35-1.85 (m, 11H), 1.85-1.40 (m, 30H), 1.40-1.12 (m, 19H), 1.09-0.85 (m, 21H), 0.76-0.52 (m, 2H).

Step 3: (27E,29E,31E,32E,39R,40S,41R,44R,46S,48S,50S,51S,53R,54R,63R)-51-[(1R)-2-[(1S,3R,4R)-4-[3-[(2S,6R)-2,6-dimethylmorpholin-4-yl]propoxy]-3-methoxy-cyclohexyl]-1-methyl-ethyl]-53,63-dihydroxy-54-methoxy-39,40,41,44,55,56-hexamethyl-50-[2-(oxetan-3-yloxy)ethoxy]-75,76-dioxa-64-azatricyclohexatriaconta-27,29,31(55),32(56)-tetraene-57,58,59,60,61-pentone (I-101) and (27E,29E,31E,32E,39R,40S,41R,44R,46S,48S,50R,51S,53R,54R,63R)-51-[(1R)-2-[(1S,3R,4R)-4-[3-[(2S,6R)-2,6-dimethylmorpholin-4-yl]propoxy]-3-methoxy-cyclohexyl]-1-methyl-ethyl]-53,63-dihydroxy-54-methoxy-39,40,41,44,55,56-hexamethyl-50-[2-(oxetan-3-yloxy)ethoxy]-75,76-dioxa-64-azatricyclohexatriaconta-27,29,31(55),32(56)-tetraene-57,58,59,60,61-pentone (I-100). 94 mg of I-109 was separated via chiral preparatory HPLC to provide (27E,29E,31E,32E,39R,40S,41R,44R,46S,48S,50S,51S,53R,54R,63R)-51-[(1R)-2-[(1S,3R,4R)-4-[3I-101 (14 mg, 15% yield) as a white solid and I-100 (5 mg, 5% yield) as a white solid.

Chiral analysis method:

Column: CHIRALPAK IC (IC00CD-NA012)

Column size: 0.46 cm I.D.×15 cm L

Injection: 20 μl

Mobile phase: Hexane/EtOH=50/50 (V/V)

Flow rate: 0.8 mL/min

Wavelength: UV 254 nm

Temperature: Room temperature

HPLC equipment: Shimadzu LC-20AD

I-101: ESI-MS (EI⁺, m/z): 1155.8 [M+H]⁺. ¹H NMR (500 MHz, CDCl₃) δ 6.36-5.74 (m, 4H), 5.51-4.97 (m, 4H), 4.71-4.42 (m, 5H), 4.10 (d, J=5.7 Hz, 1H), 3.87-3.06 (m, 19H), 3.03-2.87 (m, 2H), 2.84-2.46 (m, 4H), 2.39-1.62 (m, 18H), 1.56-1.32 (m, 12H), 1.11-0.89 (m, 13H), 0.88-0.58 (m, 20H).
I-100: ESI-MS (EI⁺, m/z): 1155.8 [M+H]⁺. ¹H NMR (500 MHz, CDCl₃) δ 6.55-5.80 (m, 4H), 5.55-5.03 (m, 4H), 4.85-4.45 (m, 5H), 4.11 (dd, J=100.9, 30.7 Hz, 3H), 3.88-3.15 (m, 19H), 3.10-2.09 (m, 14H), 2.01-1.74 (m, 18H), 1.54-1.14 (m, 15H), 1.09-0.63 (m, 20H).

### Example 31: Synthesis of [(40S,42R,44R)-4-[(2R)-2-[(27E,29E,31E,32E,36R,37S,38R,39R,41S,43S,45S,46S,47R,48R,58R)-47,58-dihydroxy-45,48-dimethoxy-36,37,38,39,49,50-hexamethyl-51,52,53,54,55-pentaoxo-71,72-dioxa-60-azatricyclohexatriaconta-27,29,31(49),32(50)-tetraen-46-yl]propyl]-44-methoxy-42-cyclohexyl]N-methyl-N-(2-morpholinoethyl)carbamate (I-99)

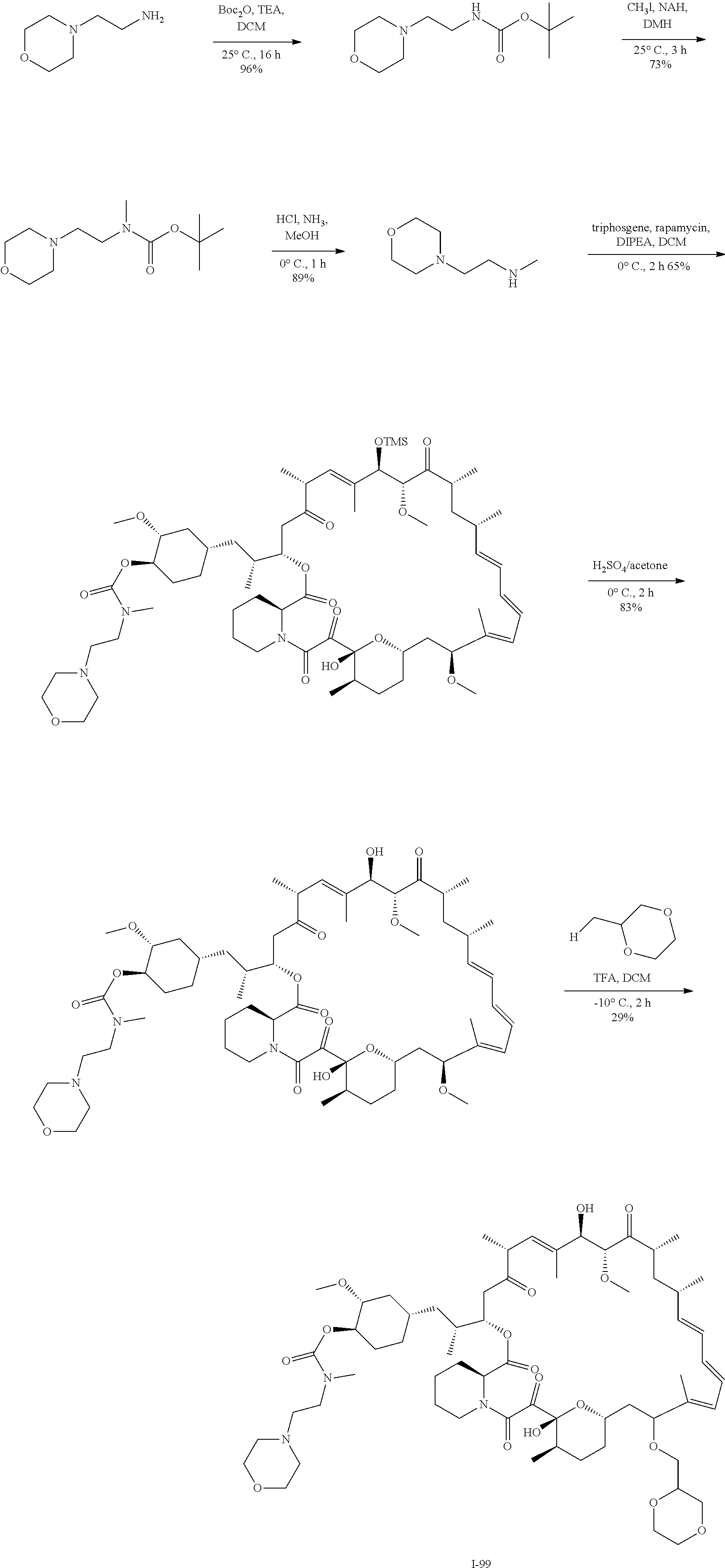

Step 1: tert-butyl N-(2-morpholinoethyl) carbamate. To a solution of 2-morpholinoethanamine (10 g, 76.81 mmol) in DCM (5 mL) was added triethylamine (5.35 mL, 38.41 mmol) and tert-butoxycarbonyl tert-butyl carbonate (18.44 g, 84.49 mmol) at 0° C. and the resulting solution was stirred overnight at 25° C. The reaction was diluted with 200 mL of dichloromethane and then washed with 30 mL of 10% sodium bicarbonate and 30 mL of brine. The organic layer was dried over sodium sulfate, filtered and concentrated to provide tert-butyl N-(2-morpholinoethyl) carbamate (17 g, 96% yield) as an off-white solid. ESI-MS (EI⁺, m/z): 231.3 [M+H]⁺. ¹H NMR (400 MHz, CDCl₃) δ 3.78-3.62 (m, 4H), 3.24 (d, J=5.5 Hz, 2H), 2.45 (dd, J=8.0, 3.9 Hz, 6H), 1.49-1.42 (m, 9H).

Step 2: tert-butyl N-methyl-N-(2-morpholinoethyl) carbamate. Tert-butyl N-(2-morpholinoethyl)carbamate (18 g, 78.16 mmol) was dissolved in DMF (240 mL), cooled to 0° C. and NaH (9.38 g, 234.47 mmol, 60% purity) was added. The reaction was stirred at room temperature for 20 minute then cooled to 0° C. and iodomethane (12.2 g, 85.97 mmol) added. The reaction mixture was stirred for 3 h then diluted with ethyl acetate (500 mL) and washed sequentially with saturated aqueous ammonium chloride solution (300 mL) and brine (300 mL×5). The organic layer was dried with sodium sulfate then concentrated to provide tert-butyl N-methyl-N-(2-morpholinoethyl) carbamate (14 g, 73% yield) as a white solid. ESI-MS (EI+, m/z): 245.3 [M+H]⁺. ¹H NMR (400 MHz, CDCl₃) δ 3.74-3.64 (m, 4H), 3.34 (s, 2H), 2.93-2.81 (m, 3H), 2.48 (d, J=4.8 Hz, 6H), 1.46 (s, 10H).

Step 3: N-methyl-2-morpholino-ethanamine. To tert-butyl N-methyl-N-(2-morpholinoethyl) carbamate (14 g, 57.30 mmol) was added hydrochloric acid (4 M, 143.25 mL) at 0° C. The reaction was stirred at room temperature for 50 min, concentrated and NH₃ (7 M, 81.86 mL) added. After stirring for 1 hr the reaction was concentrated and was purified via silica gel chromatography (DCM:MeOH:TEA=90:10:0.1) to provide the N-methyl-2-morpholino-ethanamine (7.4 g, 90% yield) as a yellow solid. ESI-MS (EI+, m/z): 145.1 [M+H]⁺. ¹H NMR (400 MHz, DMSO-d₆) δ 9.03 (s, 2H), 3.80 (s, 4H), 3.26 (dd, J=44.9, 20.4 Hz, 8H), 2.63 (s, 3H).

Step 4: [(43S,45R,47R)-4-[(2R)-2-[(30E,32E,34E,35E,39R,40S,41R,42R,44S,46S,48S,49S,50R,51R,61R)-61-hydroxy-48,51-dimethoxy-39,40,41,42,52,53-hexamethyl-54,55,56,57,58-pentaoxo-50-trimethylsilyloxy-73,74-dioxa-63-azatricyclohexatriaconta-30,32,34(52),35(53)-tetraen-49-yl]propyl]-47-methoxy-45-cyclohexyl] N-methyl-N-(2-morpholinoethyl)carbamate. To a solution of rapamycin (0.5 g, 0.507 mmol) and pyridine (2.03 mmol, 0.164 mL) in DCM (5 mL) was added triphosgene (150.43 mg, 0.507 mmol in 20 mL THF) dropwise at 0° C. under argon. The reaction mixture was stirred for 1 h at 0° C. then TEA (410 mg, 4.06 mmol) and N-methyl-2-morpholino-ethanamine (1.46 g, 10.14 mmol) were added and the resulting solution was stirred at 0° C. for a further 1 h. The reaction was diluted with DCM, washed with aqueous NH₄Cl, water, brine then dried over Na₂SO₄, filtered and concentrated. The residue was purified via silica gel chromatography (8% MeOH in DCM) to provide [(43S,45R,47R)-4-[(2R)-2-[(30E,32E,34E,35E,39R,40S,41R,42R,44S,46S,48S,49S,50R,51R,61R)-61-hydroxy-48,51-dimethoxy-39,40,41,42,52,53-hexamethyl-54,55,56,57,58-pentaoxo-50-trimethylsilyloxy-73,74-dioxa-63-azatricyclohexatriaconta-30,32,34(52),35(53)-tetraen-49-yl]propyl]-47-methoxy-45-cyclohexyl] N-methyl-N-(2-morpholinoethyl)carbamate (386 mg, 66% yield) as a light yellow solid. ESI-MS (EI⁺, m/z): 1156.4 [M+H]⁺. ¹H NMR (400 MHz, CDCl₃) δ 6.57-5.93 (m, 4H), 5.73-5.47 (m, 1H), 5.27-4.98 (m, 2H), 4.72 (s, 1H), 4.56 (s, 1H), 4.36-3.54 (m, 12H), 3.54-3.05 (m, 12H), 2.93 (s, 4H), 2.40 (dt, J=34.4, 23.8 Hz, 11H), 2.04 (s, 5H), 1.88-1.52 (m, 12H), 1.52-1.17 (m, 10H), 1.20-0.73 (m, 17H), 0.10-0.14 (m, 9H).

Step 5: [(40S,42R,44R)-4-[(2R)-2-[(27E,29E,31E,32E,36R,37S,38R,39R,41S,43S,45S,46S,47R,48R,58R)-47,58-dihydroxy-45,48-dimethoxy-36,37,38,39,49,50-hexamethyl-51,52,53,54,55-pentaoxo-71,72-dioxa-60-azatricyclohexatriaconta-27,29,31(49),32(50)-tetraen-46-yl]propyl]-44-methoxy-42-cyclohexyl] N-methyl-N-(2-morpholinoethyl)carbamate. To a solution of [(43S,45R,47R)-4-[(2R)-2-[(30E,32E,34E,35E,39R,40S,41R,42R,44S,46S,48S,49S,50R,51R,61R)-61-hydroxy-48,51-dimethoxy-39,40,41,42,52,53-hexamethyl-54,55,56,57,58-pentaoxo-50-trimethylsilyloxy-73,74-dioxa-63-azatricyclohexatriaconta-30,32,34(52),35(53)-tetraen-49-yl]propyl]-47-methoxy-45-cyclohexyl] N-methyl-N-(2-morpholinoethyl)carbamate (1.8 g, 1.56 mmol) in acetone (5 mL) and water (5 mL) was added 0.5 N sulfuric acid (0.5 M, 4.67 mL) at 0° C. The reaction was stirred at 0° C. for 2 h, and then poured into a mixture of 100 mL EtOAc and 100 mL of saturated NaHCO₃ solution. The organic layer was washed with water and brine, dried over Na₂SO₄, filtered and concentrated. The crude was purified via silica gel chromatography (5% MeOH in DCM) to provide [(40S,42R,44R)-4-[(2R)-2-[(27E,29E,31E,32E,36R,37S,38R,39R,41S,43S,45S,46S,47R,48R,58R)-47,58-dihydroxy-45,48-dimethoxy-36,37,38,39,49,50-hexamethyl-51,52,53,54,55-pentaoxo-71,72-dioxa-60-azatricyclohexatriaconta-27,29,31(49),32(50)-tetraen-46-yl]propyl]-44-methoxy-42-cyclohexyl] N-methyl-N-(2-morpholinoethyl)carbamate (1.4 g, 83% yield) as a light yellow solid. ¹H NMR (400 MHz, CDCl₃) δ 6.47-5.84 (m, 4H), 5.60-5.05 (m, 4H), 4.77 (s, 1H), 4.55 (s, 1H), 4.34-4.10 (m, 1H), 3.92-3.52 (m, 7H), 3.52-3.23 (m, 10H), 3.13 (d, J=2.7 Hz, 4H), 2.92 (s, 3H), 2.78-2.39 (m, 8H), 2.40-2.00 (m, 5H), 2.03-1.53 (m, 18H), 1.53-1.11 (m, 12H), 1.11-0.87 (m, 13H), 0.83 (d, J=6.5 Hz, 2H).

Step 6: [(43S,45R,47R)-4-[(2R)-2-[(29E,31E,33E,34E,39R,40S,41R,42R,44S,46S,49S,51R,52R,62R)-48-(1,4-dioxan-2-ylmethoxy)-51,62-dihydroxy-52-methoxy-39,40,41,42,53,54-hexamethyl-55,56,57,58,59-pentaoxo-77,78-dioxa-64-azatricyclohexatriaconta-29,31,33(53),34(54)-tetraen-49-yl]propyl]-47-methoxy-45-cyclohexyl]N-methyl-N-(2-morpholinoethyl)carbamate (I-99). To a solution of [(40S,42R,44R)-4-[(2R)-2-[(27E,29E,31E,32E,36R,37S,38R,39R,41S,43S,45S,46S,47R,48R,58R)-47,58-dihydroxy-45,48-dimethoxy-36,37,38,39,49,50-hexamethyl-51,52,53,54,55-pentaoxo-71,72-dioxa-60-azatricyclohexatriaconta-27,29,31(49),32(50)-tetraen-46-yl]propyl]-44-methoxy-42-cyclohexyl] N-methyl-N-(2-morpholinoethyl)carbamate (0.4 g, 0.37 mmol) in DCM (15 mL) was added trifluoroacetic acid (1.14 mL, 14.76 mmol) at −40° C. under N₂. then 1,4-dioxan-2-ylmethanol (0.87 g, 7.38 mmol) was added and the mixture was stirred for 2 h at −40° C. The reaction mixture was then poured into a mixture of DCM and ice cold aqueous NaHCO₃ solution and the organic layer was dried over Na₂SO₄, filtered and concentrated. The residue was purified via reverse phase chromatography (68% CH₃CN in water) to provide I-99 (70 mg, 16% yield) as a white solid. ESI-MS (EI⁺, m/z): 1170.8 [M+H]⁺. ¹H NMR (400 MHz, CDCl₃) δ 6.52-5.80 (m, 4H), 5.61-5.04 (m, 4H), 4.33 (dt, J=75.6, 73.9 Hz, 5H), 3.93-3.03 (m, 26H), 3.00-1.89 (m, 18H), 1.88-1.58 (m, 6H), 1.52-1.18 (m, 11H), 1.14-0.69 (m, 19H).

### Example 32: Synthesis (1R,9S,12S,15R,16E,18R,19R,21R,23S,24E,26E,28E,30S,32S,35R)-1-hydroxy-12-[(1R)-2-[(1S,3R,4R)-4-hydroxy-3-methoxy-cyclohexyl]-1-methyl-ethyl]-18,19-dimethoxy-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone (I-97)

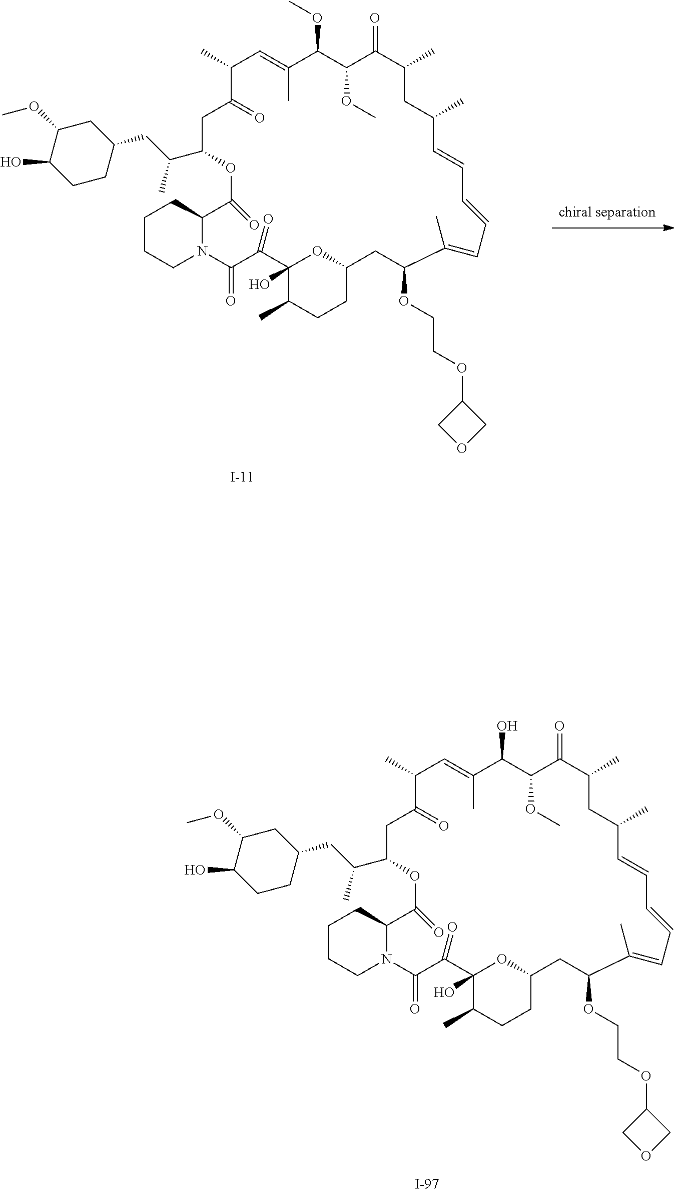

220 mg of I-11 was separated via chiral preparatory HPLC and then purified via silica gel chromatography to provide I-97 (71.5 mg, 33% yield) as a white solid.

Chiral analysis method:

Column: CHIRALPAK IC (IC00CD-TB016)

Column size: 0.46 cm I.D.×15 cm L

Injection: 80 μl

Mobile phase: Hexane/EtOH=50/50 (V/V)

Flow rate: 1.0 mL/min

Wavelength: UV 254 nm

Temperature: 35° C.

HPLC equipment: Shimadzu LC-20AT

I-97: ESI-MS (EI⁺, m/z): 1036.6 [M+Na]⁺. ¹H NMR (500 MHz, CDCl₃) δ 6.48-5.82 (m, 4H), 5.72-5.04 (m, 4H), 4.81-4.50 (m, 5H), 3.97-3.09 (m, 20H), 3.00-2.48 (m, 5H), 2.36-1.86 (m, 7H), 1.83-1.55 (m, 14H), 1.52-1.20 (m, 9H), 1.17-0.61 (m, 19H).

### Example 33: Synthesis of (25E,27E,29E,30E,36R,37S,38R,39R,41S,43S,46S,47R,48R,57R)-46-[(1R)-2-[(1S,3R,4R)-3,4-dimethoxycyclohexyl]-1-methyl-ethyl]-45-[2-[2-(dimethylamino)ethoxy]ethoxy]-57-hydroxy-47,48-dimethoxy-36,37,38,39,49,50-hexamethyl-66,67-dioxa-58-azatricyclohexatriaconta-25,27,29(49),30(50)-tetraene-51,52,53,54,55-pentone (I-95)

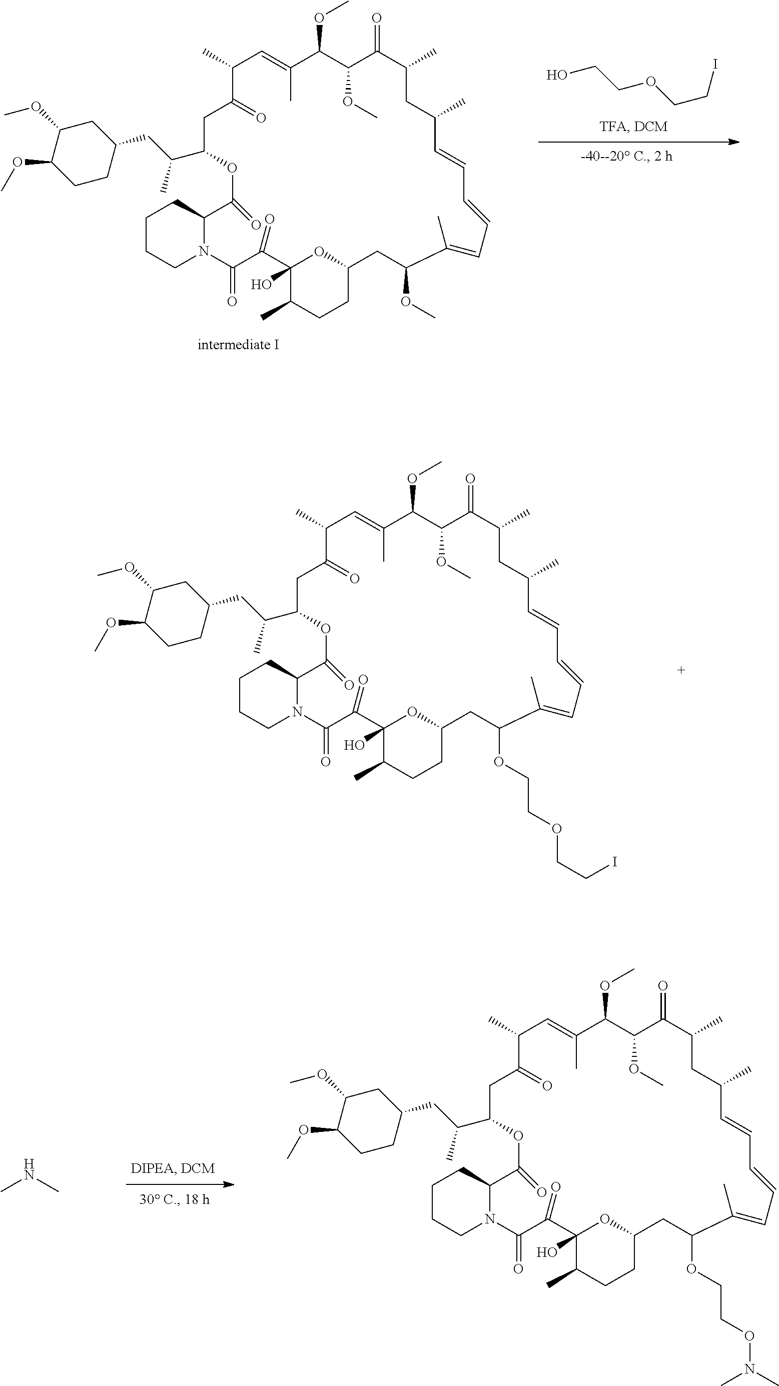

Step 1: (23E,25E,27E,28E,34R,35S,36R,37R,39S,41S,44S,45R,46R,55R)-44-[(1R)-2-[(1S,3R,4R)-3,4-dimethoxycyclohexyl]-1-methyl-ethyl]-55-hydroxy-43-[2-(2-iodoethoxy)ethoxy]-45,46-dimethoxy-34,35,36,37,47,48-hexamethyl-63,64-dioxa-56-azatricyclohexatriaconta-23,25,27(47),28(48)-tetraene-49,50,51,52,53-pentone. To a solution of (24E,26E,28E,29E,31R,32S,33R,34R,36S,38S,40S,41S,42R,43R,52R)-41-[(1R)-2-[(1S,3R,4R)-3,4-dimethoxycyclohexyl]-1-methyl-ethyl]-52-hydroxy-40,42,43-trimethoxy-31,32,33,34,44,45-hexamethyl-60,61-dioxa-53-azatricyclohexatriaconta-24,26,28(44),29(45)-tetraene-46,47,48,49,50-pentone (0.5 g, 0.53 mmol) in DCM (10 mL) under nitrogen was added TFA (1.82 g, 15.92 mmol, 1.23 mL) at −40° C. Then 2-(2-iodoethoxy)ethanol (2.29 g, 10.61 mmol) was added and the mixture was stirred at −20° C. for 3h. The reaction was quenched by adding saturated aqueous NaHCO₃ (20 mL) and extracted with DCM (30 mL) at 0° C. The organic layer was washed with water (20 mL) and brine (20 mL), dried over anhydrous sodium sulfate, filtered and concentrated. The residue was purified via silica gel chromatography (PE:EA=1:1) to provide (23E,25E,27E,28E,34R,35S,36R,37R,39S,41S,44S,45R,46R,55R)-44-[(1R)-2-[(1S,3R,4R)-3,4-dimethoxycyclohexyl]-1-methyl-ethyl]-55-hydroxy-43-[2-(2-iodoethoxy)ethoxy]-45,46-dimethoxy-34,35,36,37,47,48-hexamethyl-63,64-dioxa-56-azatricyclohexatriaconta-23,25,27(47),28(48)-tetraene-49,50,51,52,53-pentone (0.2 g, 33% yield) as a white solid. ESI-MS (EI⁺, m/z): 1148.4 [M+Na]⁺. ¹H NMR (400 MHz, CDCl₃) δ 6.46-5.97 (m, 4H), 5.71-5.03 (m, 4H), 4.19-4.04 (m, 1H), 3.93-3.53 (m, 7H), 3.50-3.38 (m, 8H), 3.37-3.21 (m, 7H), 3.20-2.97 (m, 6H), 2.96-2.50 (m, 4H), 2.40-2.19 (m, 4H), 2.18-1.85 (m, 6H), 1.82-1.55 (m, 13H), 1.53-1.21 (m, 10H), 1.20-0.81 (m, 13H), 0.79-0.69 (m, 1H).

Step 2: (25E,27E,29E,30E,36R,37S,38R,39R,41S,43S,46S,47R,48R,57R)-46-[(1R)-2-[(1S,3R,4R)-3,4-dimethoxycyclohexyl]-1-methyl-ethyl]-45-[2-[2-(dimethylamino)ethoxy]ethoxy]-57-hydroxy-47,48-dimethoxy-36,37,38,39,49,50-hexamethyl-66,67-dioxa-58-azatricyclohexatriaconta-25,27,29(49),30(50)-tetraene-51,52,53,54,55-pentone (I-95). A solution of (23E,25E,27E,28E,34R,35S,36R,37R,39S,41S,44S,45R,46R,55R)-44-[(1R)-2-[(1S,3R,4R)-3,4-dimethoxycyclohexyl]-1-methyl-ethyl]-55-hydroxy-43-[2-(2-iodoethoxy)ethoxy]-45,46-dimethoxy-34,35,36,37,47,48-hexamethyl-63,64-dioxa-56-azatricyclohexatriaconta-23,25,27(47),28(48)-tetraene-49,50,51,52,53-pentone (0.3 g, 0.27 mmol), N-methylmethanamine (120 mg, 2.66 mmol) and N-ethyl-N-isopropyl-propan-2-amine (344 mg, 2.66 mmol) in DCM (5 mL) was stirred for 18 h at 30° C. The reaction mixture was diluted with DCM (10 mL) and washed with saturated NH₄Cl (10 mL), water (10 mL) and brine (10 mL), dried over anhydrous sodium sulfate, filtered and concentrated. The residue was purified via reverse-phase chromatography eluting with 50% CH₃CN in water to provide I-95 (70 mg, 25% yield) as a white solid. ESI-MS (EI⁺, m/z): 1044.7 [M+H]⁺. ¹H NMR (400 MHz, CDCl₃) δ 6.81-6.70 (m, 1H), 6.39-5.87 (m, 4H), 5.58-5.39 (m, 2H), 4.50-3.91 (m, 4H), 3.87-3.49 (m, 7H), 3.48-3.35 (m, 7H), 3.34-3.20 (m, 5H), 3.19-2.97 (m, 6H), 2.90-2.76 (m, 6H), 2.69-1.97 (m, 17H), 1.88-1.40 (m, 11H), 1.37-0.92 (m, 21H), 0.91-0.77 (m, 3H).

### Example 34: Synthesis of (25E,27E,29E,30E,36R,37S,38R,39R,41S,43S,46S,48R,49R,58R)-58-hydroxy-48,49-dimethoxy-46-[(1R)-2-[(1S,3R,4R)-3-methoxy-4-(2-methoxyethoxy)cyclohexyl]-1-methyl-ethyl]-36,37,38,39,50,51-hexamethyl-45-[2-(oxetan-3-yloxy)ethoxy]-67,68-dioxa-59-azatricyclohexatriaconta-25,27,29(50),30(51)-tetraene-52,53,54,55,56-pentone (I-94), (1R,9S,12S,15R,16E,18R,19R,21R,23S,24E,26E,28E,30S,32S,35R)-1-hydroxy-18,19-dimethoxy-12-[(1R)-2-[(1S,3R,4R)-3-methoxy-4-(2-methoxyethoxy)cyclohexyl]-1-methyl-ethyl]-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone (I-83) and (1R,9S,12S,15R,16E,18R,19R,21R,23S,24E,26E,28E,30R,32S,35R)-1-hydroxy-18,19-dimethoxy-12-[(1R)-2-[(1S,3R,4R)-3-methoxy-4-(2-methoxyethoxy)cyclohexyl]-1-methyl-ethyl]-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone (I-82)

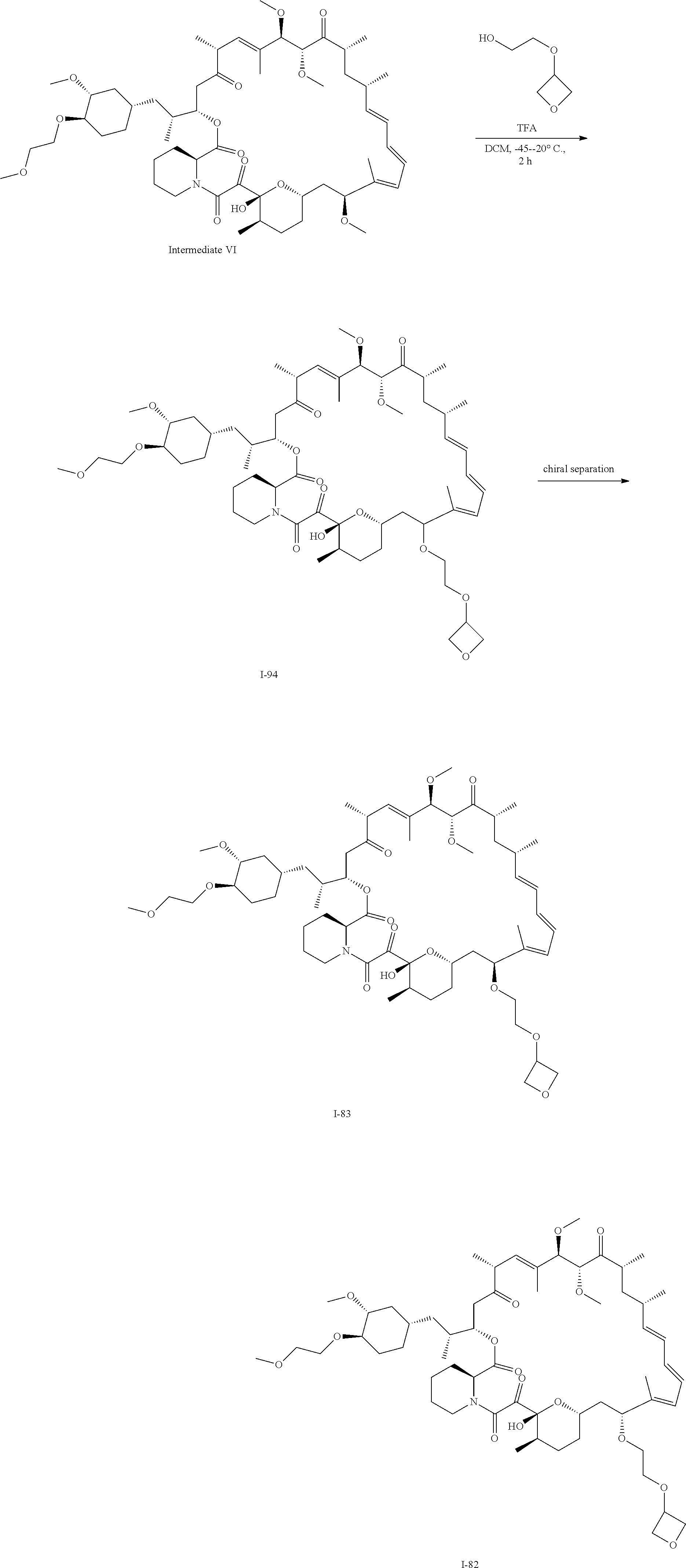

Step 1: (25E,27E,29E,30E,36R,37S,38R,39R,41S,43S,46S,48R,49R,58R)-58-hydroxy-48,49-dimethoxy-46-[(1R)-2-[(1S,3R,4R)-3-methoxy-4-(2-methoxyethoxy)cyclohexyl]-1-methyl-ethyl]-36,37,38,39,50,51-hexamethyl-45-[2-(oxetan-3-yloxy)ethoxy]-67,68-dioxa-59-azatricyclohexatriaconta-25,27,29(50),30(51)-tetraene-52,53,54,55,56-pentone (I-94). To a solution of (24E,26E,28E,29E,33R,34S,35R,36R,38S,40S,42S,43S,44R,45R,54R)-54-hydroxy-42,44,45-trimethoxy-43-[(1R)-2-[(1S,3R,4R)-3-methoxy-4-(2-methoxyethoxy)cyclohexyl]-1-methyl-ethyl]-33,34,35,36,46,47-hexamethyl-62,63-dioxa-55-azatricyclohexatriaconta-24,26,28(46),29(47)-tetraene-48,49,50,51,52-pentone (Intermediate VI, 0.62 g, 0.63 mmol) in DCM (30 mL) was added 2,2,2-trifluoroacetic acid (1.43 g, 12.57 mmol, 0.97 mL) dropwise at −55° C. under N₂. The reaction was stirred for 10 min at −45° C., 2-(oxetan-3-yloxy) ethanol (1.49 g, 12.57 mmol in DCM) was added and the mixture stirred for 2 h at −45° C. The reaction mixture was poured into saturated aqueous NaHCO₃ (40 mL) at 0° C. and extracted with DCM (40 mL). The organic layer was washed with water (40 mL) and brine (40 mL), dried over anhydrous sodium sulfate, filtered and concentrated. The residue was purified via silica gel chromatography (EA:PE=9:1) then reverse-phase chromatography (eluting with 40% CH₃CN in water) to provide I-94 (0.074 g, 11% yield) as a white solid. ESI-MS (EI⁺, m/z): 1094.8 [M+H]⁺. ¹H NMR (400 MHz, CDCl₃) δ 6.45-5.98 (m, 4H), 5.66-4.97 (m, 4H), 4.43-4.78 (m, 5H), 4.31-4.18 (m, 1H), 3.91-3.69 (m, 4H), 3.67-3.24 (m, 17H), 3.21-2.99 (m, 5H), 2.86-2.50 (m, 3H), 2.30-1.84 (m, 6H), 1.78-1.59 (m, 20H), 1.51-1.23 (m, 10H), 1.20-1.03 (m, 11H), 0.97-0.84 (m, 8H), 0.78-0.69 (m, 1H).

Step 2: (1R,9S,12S,15R,16E,18R,19R,21R,23S,24E,26E,28E,30S,32S,35R)-1-hydroxy-18,19-dimethoxy-12-[(1R)-2-[(1S,3R,4R)-3-methoxy-4-(2-methoxyethoxy)cyclohexyl]-1-methyl-ethyl]-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone (I-83) and (1R,9S,12S,15R,16E,18R,19R,21R,23S,24E,26E,28E,30R,32S,35R)-1-hydroxy-18,19-dimethoxy-12-[(1R)-2-[(1S,3R,4R)-3-methoxy-4-(2-methoxyethoxy)cyclohexyl]-1-methyl-ethyl]-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone (I-82). 130 mg of I-94 was separated via chiral preparatory HPLC and then purified via silica gel chromatography to obtain I-83 (50.8 mg, 39% yield) as a white solid and I-82 (6.2 mg, 5% yield) as a white solid.

Chiral analysis method:

Column: CHIRALPAK IC (IC00CD-TB016)

Column size: 0.46 cm I.D.×15 cm L

Injection: 10 μl

Mobile phase: Hexane/EtOH=50/50 (V/V)

Flow rate: 1.0 mL/min

Wavelength: UV 254 nm

Temperature: 35° C.

HPLC equipment: Shimadzu-LC-20AD

I-83: ESI-MS (EI⁺, m/z): 1094.7 [M+H]⁺. ¹H NMR (500 MHz, CDCl₃) δ 6.46-5.82 (m, 4H), 5.71-5.02 (m, 4H), 4.81-4.49 (m, 5H), 4.00-3.20 (m, 24H), 3.19-2.98 (m, 5H), 2.95-2.43 (m, 3H), 2.36-1.84 (m, 7H), 1.80-1.56 (m, 13H), 1.52-1.22 (m, 9H), 1.19-0.68 (m, 19H).
I-82: ESI-MS (EI⁺, m/z): 1094.6 [M+H]⁺. ¹H NMR (500 MHz, CDCl₃) δ 6.46-5.84 (m, 4H), 5.78-5.02 (m, 4H), 4.74-4.42 (m, 5H), 3.91-2.79 (m, 29H), 2.72-1.56 (m, 24H), 1.46-0.59 (m, 27H).

### Example 35: Synthesis of (24E,26E,28E,29E,35R,36S,37R,38R,40S,42S,45S,47R,48R,57R)-44-(1,4-dioxan-2-ylmethoxy)-47,57-dihydroxy-45-[(1R)-2-[(1S,3R,4R)-4-(3-hydroxypropoxy)-3-methoxy-cyclohexyl]-1-methyl-ethyl]-48-methoxy-35,36,37,38,49,50-hexamethyl-69,70-dioxa-58-azatricyclohexatriaconta-24,26,28(49),29(50)-tetraene-51,52,53,54,55-pentone (I-106), (24E,26E,28E,29E,35R,36S,37R,38R,40S,42S,44S,45S,47R,48R,57R)-44-(1,4-dioxan-2-ylmethoxy)-47,57-dihydroxy-45-[(1R)-2-[(1S,3R,4R)-4-(3-hydroxypropoxy)-3-methoxy-cyclohexyl]-1-methyl-ethyl]-48-methoxy-35,36,37,38,49,50-hexamethyl-69,70-dioxa-58-azatricyclohexatriaconta-24,26,28(49),29(50)-tetraene-51,52,53,54,55-pentone (I-93) and (24E,26E,28E,29E,35R,36S,37R,38R,40S,42S,44R,45S,47R,48R,57R)-44-(1,4-dioxan-2-ylmethoxy)-47,57-dihydroxy-45-[(1R)-2-[(1S,3R,4R)-4-(3-hydroxypropoxy)-3-methoxy-cyclohexyl]-1-methyl-ethyl]-48-methoxy-35,36,37,38,49,50-hexamethyl-69,70-dioxa-58-azatricyclohexatriaconta-24,26,28(49),29(50)-tetraene-51,52,53,54,55-pentone (I-92)

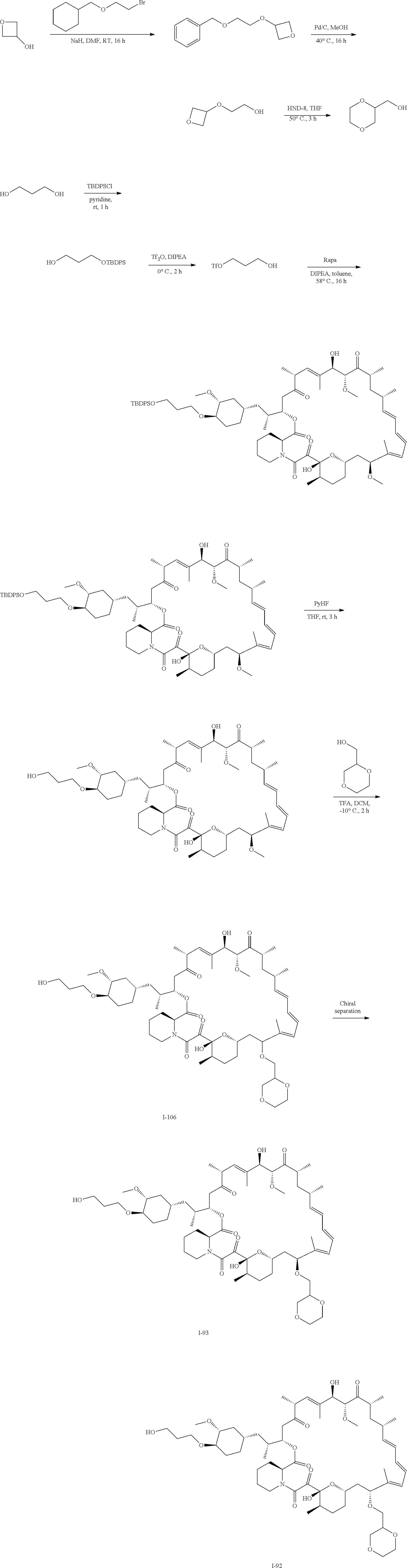

Step 1: 3-(2-benzyloxyethoxy) oxetane. To a solution of oxetan-3-ol (10 g, 135 mmol) in DMF (160 mL) was added sodium hydride (3.24 g, 135 mmol) at 0° C., the resulting solution was stirred at this temperature for 30 min, then 2-bromoethoxymethylbenzene (43.55 g, 202.49 mmol) was added. The reaction was stirred for 2 h at 0° C. in an ice water bath then warmed to rt and was stirred for 16 h. The reaction was quenched with 1200 mL of NH₄Cl (sat., aq.), extracted with ethyl acetate (2×120 mL) and the organic layers combined and concentrated. The residue was purified via silica gel chromatography with PE/EA (8:1) to provide 3-(2-benzyloxyethoxy) oxetane (16.4 g, 58% yield) as a colorless liquid. ESI-MS (EI⁺, m/z): 231 [M+Na]⁺. ¹H NMR (400 MHz, CDCl₃) δ 7.41-7.23 (m, 6H), 4.79-4.70 (m, 2H), 4.68-4.52 (m, 6H), 3.62-3.53 (m, 4H).

Step 2: 2-(oxetan-3-yloxy) ethanol. To a solution of 3-(2-benzyloxyethoxy)oxetane (4 g, 19.21 mmol) in MeOH (20 mL) was added palladium (10% on carbon) (2.04 g, 19.21 mmol) under N₂. The solution was stirred under H₂ at 40° C. overnight then filtered and concentrated. The residue was purified via silica gel chromatography (PE:EA=1:5) to provide 2-(oxetan-3-yloxy)ethanol (2.1 g, 93% yield) as a colorless liquid. ¹HNMR (400 MHz, CDCl₃) δ 4.79 (td, J=5.8, 2.1 Hz, 2H), 4.62 (dt, J=10.2, 4.9 Hz, 3H), 3.80-3.69 (m, 2H), 3.52-3.44 (m, 2H), 2.36 (s, 1H).

Step 3: 1, 4-dioxan-2-ylmethanol. A mixture of 2-(oxetan-3-yloxy)ethanol (4 g, 33.86 mmol) and HND-8 (1.2 g) in THF (60 mL) was stirred at 50° C. for 3 hr. The mixture was filtered and concentrated to provide 1,4-dioxan-2-ylmethanol (3.66 g, 92% yield) as a colorless oil. ¹HNMR (400 MHz, CDCl₃) δ 3.86-3.42 (m, 9H), 1.98 (s, 1H).

Step 4: 2-[tert-butyl (diphenyl) silyl] oxyethyl trifluoromethanesulfonate. To a solution of 2-[tert-butyl(diphenyl)silyl]oxyethanol (7 g, 23.3 mmol) and DIEA (4.52 g, 34.95 mmol) in DCM (20 mL) at 0° C. under N₂ was added trifluoromethylsulfonyl trifluoromethanesulfonate (7.23 g, 25.63 mmol). The mixture was stirred at 0° C. for 2 h then diluted with DCM (150 mL), washed with saturated NaHCO₃ (50 mL), water (50 mL), brine (50 mL), dried over Na₂SO₄, filtered and concentrated to provide 2-[tert-butyl(diphenyl)silyl]oxyethyl trifluoromethanesulfonate as a brown oil. This was used without further purification. ¹H NMR (400 MHz, CDCl₃) δ 7.68-7.65 (m, 4H), 7.47-7.38 (m, 6H), 4.59-4.53 (m, 2H), 3.94-3.86 (m, 2H), 1.07 (d, J=5.4 Hz, 9H).

Step 5: (35E,37E,39E,40E,46R,47S,48R,49R,51S,53S,55S,56S,57R,58R,67R)-56-[(1R)-2-[(1S,3R,4R)-4-[2-[tert-butyl(diphenyl)silyl]oxyethoxy]-3-methoxy-cyclohexyl]-1-methyl-ethyl]-57,67-dihydroxy-55,58-dimethoxy-46,47,48,49,59,60-hexamethyl-77,78-dioxa-69-azatricyclohexatriaconta-35,37,39(59),40(60)-tetraene-61,62,63,64,65-pentone. A mixture of rapamycin (2 g, 2.19 mmol), 2-[tert-butyl(diphenyl)silyl]oxyethyl trifluoromethanesulfonate (9.46 g, 21.88 mmol) and N-ethyl-N-isopropyl-propan-2-amine (3.39 g, 26.25 mmol) in toluene (20 mL) was stirred at 58° C. for 18 h then poured into cold saturated NaHCO₃ (150 mL). This was extracted with EtOAc (200 mL) and the organic layer washed with water (150 mL×3) and brine (150 mL), dried over anhydrous Na₂SO₄, filtered and concentrated. The residue was purified via silica gel chromatography (PE:EA=3:1) to provide (35E,37E,39E,40E,46R,47S,48R,49R,51S,53S,55S,56S,57R,58R,67R)-56-[(1R)-2-[(1S,3R,4R)-4-[2-[tert-butyl(diphenyl)silyl]oxyethoxy]-3-methoxy-cyclohexyl]-1-methyl-ethyl]-57,67-dihydroxy-55,58-dimethoxy-46,47,48,49,59,60-hexamethyl-77,78-dioxa-69-azatricyclohexatriaconta-35,37,39(59),40(60)-tetraene-61,62,63,64,65-pentone as a yellow solid. ESI-MS (EI⁺, m/z):1218.6 [M+Na]⁺.

Step 6: (22E,24E,26E,27E,32R,33S,34R,35R,37S,39S,41S,42S,43R,44R,53R)-43,53-dihydroxy-42-[(1R)-2-[(1S,3R,4R)-4-(3-hydroxypropoxy)-3-methoxy-cyclohexyl]-1-methyl-ethyl]-41,44-dimethoxy-32,33,34,35,45,46-hexamethyl-63,64-dioxa-54-azatricyclohexatriaconta-22,24,26(45),27(46)-tetraene-47,48,49,50,51-pentone. To a solution of (35E,37E,39E,40E,47R,48S,49R,50R,52S,54S,56S,57S,58R,59R,68R)-57-[(1R)-2-[(1S,3R,4R)-4-[3-[tert-butyl(diphenyl)silyl]oxypropoxy]-3-methoxy-cyclohexyl]-1-methyl-ethyl]-58,68-dihydroxy-56,59-dimethoxy-47,48,49,50,60,61-hexamethyl-78,79-dioxa-70-azatricyclohexatriaconta-35,37,39(60),40(61)-tetraene-62,63,64,65,66-pentone (3.46 g, 2.86 mmol) in THF (70 mL) at 0° C. was added pyridine HF (2.26 g, 28.58 mmol). The mixture was stirred at 30° C. for 3 h then cooled to 0° C. and quenched with saturated aqueous NaHCO₃ (20 mL) and extracted with EA (30 mL). The organic layer was washed with water (100 mL) and brine (100 mL), dried over anhydrous sodium sulfate, filtered and concentrated. The residue was purified via silica gel chromatography (PE:EA=2:3) to provide (22E,24E,26E,27E,32R,33S,34R,35R,37S,39S,41S,42S,43R,44R,53R)-43,53-dihydroxy-42-[(1R)-2-[(1S,3R,4R)-4-(3-hydroxypropoxy)-3-methoxy-cyclohexyl]-1-methyl-ethyl]-41,44-dimethoxy-32,33,34,35,45,46-hexamethyl-63,64-dioxa-54-azatricyclohexatriaconta-22,24,26(45),27(46)-tetraene-47,48,49,50,51-pentone as a light yellow solid. ESI-MS (EI⁺, m z):994.5 [M+Na]

Step 7: (24E,26E,28E,29E,35R,36S,37R,38R,40S,42S,45S,47R,48R,57R)-44-(1,4-dioxan-2-ylmethoxy)-47,57-dihydroxy-45-[(1R)-2-[(1S,3R,4R)-4-(3-hydroxypropoxy)-3-methoxy-cyclohexyl]-1-methyl-ethyl]-48-methoxy-35,36,37,38,49,50-hexamethyl-69,70-dioxa-58-azatricyclohexatriaconta-24,26,28(49),29(50)-tetraene-51,52,53,54,55-pentone (I-106). To a solution of (22E,24E,26E,27E,32R,33S,34R,35R,37S,39S,41S,42S,43R,44R,53R)-43,53-dihydroxy-42-[(1R)-2-[(1S,3R,4R)-4-(3-hydroxypropoxy)-3-methoxy-cyclohexyl]-1-methyl-ethyl]-41,44-dimethoxy-32,33,34,35,45,46-hexamethyl-63,64-dioxa-54-azatricyclohexatriaconta-22,24,26(45),27(46)-tetraene-47,48,49,50,51-pentone (0.6 g, 0.62 mmol) and 1,4-dioxan-2-ylmethanol (2.19 g, 18.51 mmol) in DCM (40 mL) at −40° C. under N₂ was added 2,2,2-trifluoroacetic acid (2.81 g, 24.69 mmol). The reaction mixture was stirred for 2 h at −10° C. then washed with ice cold saturated NaHCO₃ (100 mL), water (100 mL×3) and brine (100 mL×3), dried over anhydrous sodium sulfate, filtered and concentrated. The residue was purified via reverse phase column chromatography eluting with 65% CH₃CN in water to provide I-106. ESI-MS (EI⁺, m/z):1080.4 [M+Na]⁺. ¹H NMR (400 MHz, CDCl₃) δ 6.44-5.89 (m, 4H), 5.61-5.37 (m, 2H), 5.31-5.12 (m, 2H), 4.79 (d, J=18.7 Hz, 1H), 4.29 (d, J=12.8 Hz, 1H), 3.95-3.53 (m, 11H), 3.53-3.27 (m, 9H), 3.27-2.96 (m, 5H), 2.71 (s, 1H), 2.58 (d, J=13.9 Hz, 1H), 2.34 (d, J=11.8 Hz, 2H), 2.08 (s, 3H), 1.87-1.57 (m, 20H), 1.47 (dd, J=22.8, 10.6 Hz, 3H), 1.26-0.77 (m, 19H), 0.70 (dd, J=23.6, 12.0 Hz, 1H).

Step 8: (24E,26E,28E,29E,35R,36S,37R,38R,40S,42S,44S,45S,47R,48R,57R)-44-(1,4-dioxan-2-ylmethoxy)-47,57-dihydroxy-45-[(1R)-2-[(1S,3R,4R)-4-(3-hydroxypropoxy)-3-methoxy-cyclohexyl]-1-methyl-ethyl]-48-methoxy-35,36,37,38,49,50-hexamethyl-69,70-dioxa-58-azatricyclohexatriaconta-24,26,28(49),29(50)-tetraene-51,52,53,54,55-pentone (I-93) and (24E,26E,28E,29E,35R,36S,37R,38R,40S,42S,44R,45S,47R,48R,57R)-44-(1,4-dioxan-2-ylmethoxy)-47,57-dihydroxy-45-[(1R)-2-[(1S,3R,4R)-4-(3-hydroxypropoxy)-3-methoxy-cyclohexyl]-1-methyl-ethyl]-48-methoxy-35,36,37,38,49,50-hexamethyl-69,70-dioxa-58-azatricyclohexatriaconta-24,26,28(49),29(50)-tetraene-51,52,53,54,55-pentone (I-92). 240 mg of I-106 was separated via chiral preparatory HPLC and then purified via silica gel chromatography (9% MeOH in a mixture of DCM:PE:EA=3:3:1) to provide I-93 (45 mg, 19% yield) as a white solid and I-92 (25 mg, 10% yield) as a white solid.

Chiral analysis method:

Column: CHIRALPAK IC (IC00CD-TB016)

Column size: 0.46 cm I.D.×15 cm L

Injection: 100 μl

Mobile phase: Hexane/EtOH=60/40(V/V)

Flow rate: 1.0 mL/min

Wavelength: UV 254 nm

Temperature: 35° C.

HPLC equipment: Shimadzu LC-20AT

I-93: ESI-MS (EI⁺, m/z):1080.8 [M+Na]⁺. ¹H NMR (400 MHz, CDCl₃) δ 6.37-5.74 (m, 4H), 5.51-5.00 (m, 4H), 4.10 (d, J=6.0 Hz, 1H), 3.86-3.44 (m, 14H), 3.40-2.90 (m, 15H), 2.82-2.45 (m, 3H), 2.34-1.34 (m, 24H), 1.30-1.10 (m, 7H), 1.05-0.53 (m, 19H).
I-92: ESI-MS (EI⁺, m/z):1080.7 [M+Na]⁺. ¹H NMR (400 MHz, CDCl₃) δ 6.46-5.88 (m, 4H), 5.70-5.06 (m, 4H), 4.23 (t, J=32.2 Hz, 2H), 4.01-3.52 (m, 12H), 3.52-2.88 (m, 16H), 2.78-1.98 (m, 9H), 1.92-1.72 (m, 8H), 1.51-1.20 (m, 17H), 1.14-0.60 (m, 19H).

### Example 36: Synthesis of (27E,29E,31E,32E,39R,40S,41R,42R,44S,46S,49S,51R,52R,62R)-51,62-dihydroxy-52-methoxy-49-[(1R)-2-[(1S,3R,4R)-3-methoxy-4-[3-(2-oxa-6-azaspiro[3.3]heptan-6-yl)propoxy]cyclohexyl]-1-methyl-ethyl]-39,40,41,42,53,54-hexamethyl-48-[2-(oxetan-3-yloxy)ethoxy]-74,75-dioxa-63-azatricyclohexatriaconta-27,29,31(53),32(54)-tetraene-55,56,57,58,59-pentone (I-91)

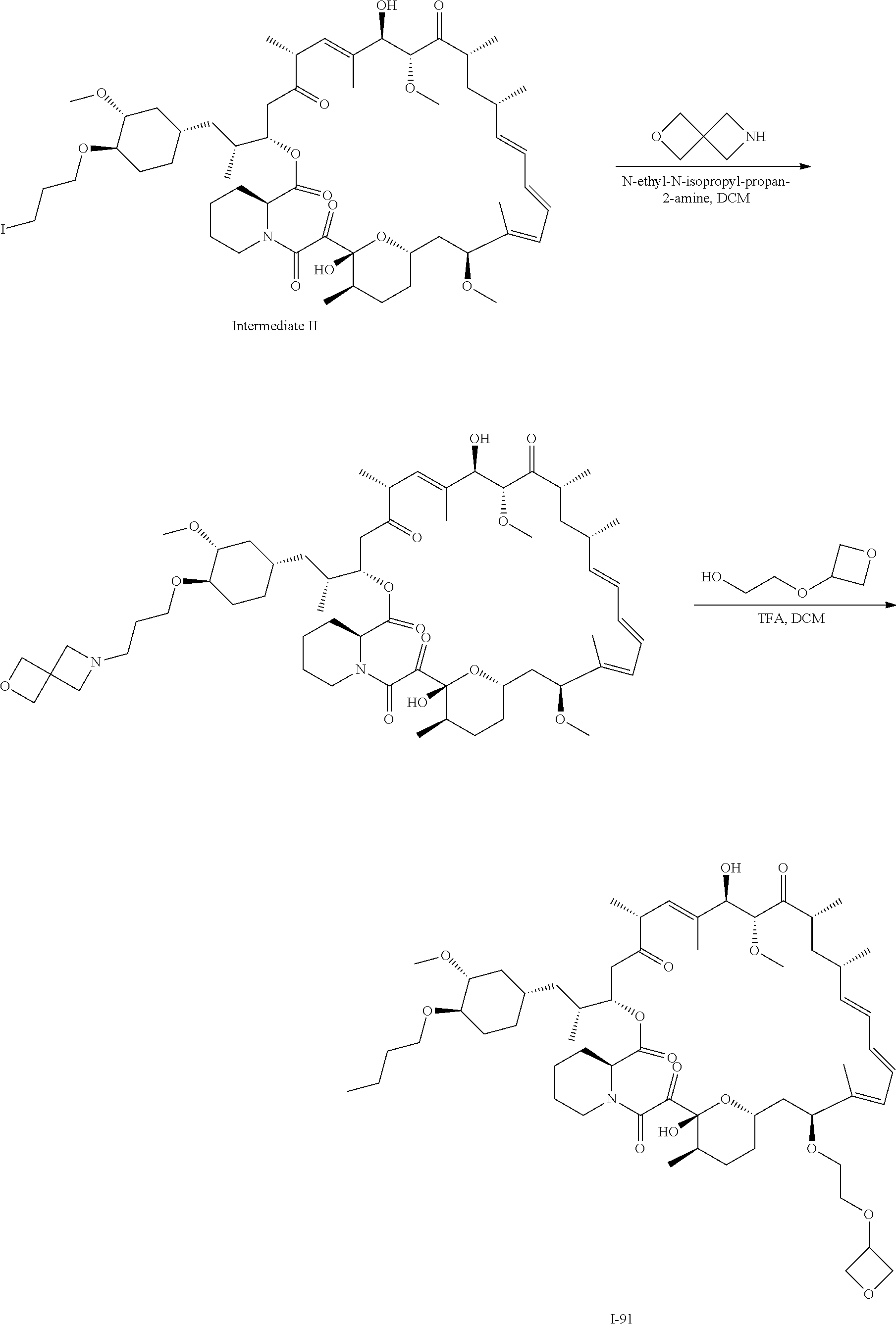

Step 1: (26E,28E,30E,31E,36R,37S,38R,39R,41S,43S,45S,46S,47R,48R,58R)-47,58-dihydroxy-45,48-dimethoxy-46-[(1R)-2-[(1S,3R,4R)-3-methoxy-4-[3-(2-oxa-6-azaspiro[3.3]heptan-6-yl)propoxy]cyclohexyl]-1-methyl-ethyl]-36,37,38,39,49,50-hexamethyl-69,70-dioxa-59-azatricyclohexatriaconta-26,28,30(49),31(50)-tetraene-51,52,53,54,55-pentone. A solution of Intermediate II (2.9 g, 2.68 mmol), 2-oxa-6-azaspiro[3.3]heptane (0.797 g, 8.04 mmol, 0.123 mL) and N-ethyl-N-isopropyl-propan-2-amine (1.04 g, 8.04 mmol, 1.40 mL) in DCM (50 mL) was stirred for 20 h at 22° C. The reaction mixture was diluted with DCM (10 mL) and washed with saturated NH₄Cl (10 mL×3), water (10 mL×3) and brine (10 mL×3), dried over anhydrous sodium sulfate, filtered and concentrated. The residue was purified via reverse-phase chromatography eluting with 50% CH₃CN in water to provide (26E,28E,30E,31E,36R,37S,38R,39R,41S,43S,45S,46S,47R,48R,58R)-47,58-dihydroxy-45,48-dimethoxy-46-[(1R)-2-[(1S,3R,4R)-3-methoxy-4-[3-(2-oxa-6-azaspiro[3.3]heptan-6-yl)propoxy]cyclohexyl]-1-methyl-ethyl]-36,37,38,39,49,50-hexamethyl-69,70-dioxa-59-azatricyclohexatriaconta-26,28,30(49),31(50)-tetraene-51,52,53,54,55-pentone (1.5 g, 53% yield) as a white solid. ESI-MS (EI+, m/z): 1053.8 [M+H]+, T=1.882 min, 100% purity, 254 nm.

Step 2: (26E,28E,30E,31E,36R,37S,38R,39R,41S,43S,45S,46S,47R,48R,58R)-47,58-dihydroxy-45,48-dimethoxy-46-[(1R)-2-[(1S,3R,4R)-3-methoxy-4-[3-(2-oxa-6-azaspiro[3.3]heptan-6-yl)propoxy]cyclohexyl]-1-methyl-ethyl]-36,37,38,39,49,50-hexamethyl-69,70-dioxa-59-azatricyclohexatriaconta-26,28,30(49),31(50)-tetraene-51,52,53,54,55-pentone (I-91). To a solution of (26E,28E,30E,31E,36R,37S,38R,39R,41S,43S,45S,46S,47R,48R,58R)-47,58-dihydroxy-45,48-dimethoxy-46-[(1R)-2-[(1S,3R,4R)-3-methoxy-4-[3-(2-oxa-6-azaspiro[3.3]heptan-6-yl)propoxy]cyclohexyl]-1-methyl-ethyl]-36,37,38,39,49,50-hexamethyl-69,70-dioxa-59-azatricyclohexatriaconta-26,28,30(49),31(50)-tetraene-51,52,53,54,55-pentone (0.5 g, 0.47 mmol) in DCM (25 mL) was added 2,2,2-trifluoroacetic acid (1.89 g, 16.61 mmol, 1.28 mL) dropwise at −55° C. under N₂. The reaction was stirred for 10 min at −45° C. then 2-(oxetan-3-yloxy)ethanol (1.12 g, 9.49 mmol in DCM) was added and the reaction stirred for a further 2 h at −20° C. The reaction mixture was poured into saturated NaHCO₃ (40 mL) at 0° C. and extracted with DCM (40 mL). The organic layer was washed with water (40 mL) and brine (40 mL), dried over anhydrous sodium sulfate, filtered and concentrated. The residue was purified by reverse phase chromatography eluting with 40% CH₃CN in water (0.01% HCOOH to provide I-91 (0.06 g, 11% yield) as a white solid. ESI-MS (EI⁺, m/z): 1139.8 [M+H]⁺, T=1.814 min, 98% purity, 254 nm. ¹H NMR (400 MHz, CDCl₃) δ 6.40-5.95 (m, 4H), 5.54-5.12 (m, 4H), 4.85 (br, 4H), 4.79-4.54 (m, 5H), 4.45-4.03 (m, 6H), 3.94-3.64 (m, 4H), 3.57-3.19 (m, 15H), 3.13-2.95 (m, 6H), 2.77-2.13 (m, 6H), 2.03-1.56 (m, 27H), 1.53-1.37 (m, 5H), 1.38-081 (m, 33H), 0.69-0.61 (m, 3H).

### Example 37: Synthesis of (24E,26E,28E,29E,35R,36S,37R,38R,40S,42S,45S,46R,47R,56R)-45-[(1R)-2-[(1S,3R,4R)-3,4-dimethoxycyclohexyl]-1-methyl-ethyl]-56-hydroxy-46,47-dimethoxy-35,36,37,38,48,49-hexamethyl-44-[2-[2-(methylamino)ethoxy]ethoxy]-65,66-dioxa-58-azatricyclohexatriaconta-24,26,28(48),29(49)-tetraene-50,51,52,53,54-pentone (I-90)

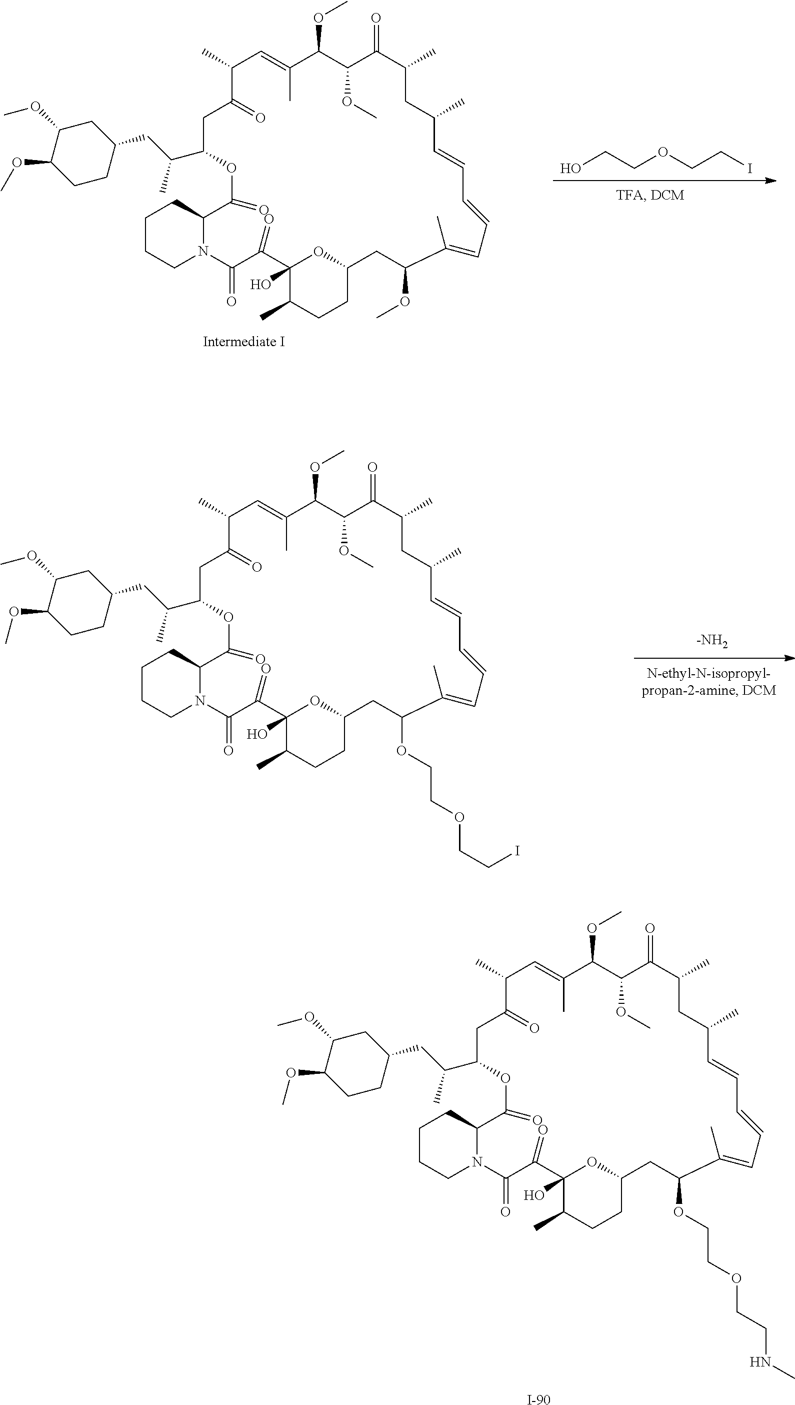

Step 1: (23E,25E,27E,28E,34R,35S,36R,37R,39S,41S,44S,45R,46R,55R)-44-[(1R)-2-[(1S,3R,4R)-3,4-dimethoxycyclohexyl]-1-methyl-ethyl]-55-hydroxy-43-[2-(2-iodoethoxy)ethoxy]-45,46-dimethoxy-34,35,36,37,47,48-hexamethyl-63,64-dioxa-56-azatricyclohexatriaconta-23,25,27(47),28(48)-tetraene-49,50,51,52,53-pentone. To a solution of Intermediate I (2.9 g, 2.68 mmol) in DCM (10 mL) under nitrogen was added TFA (1.82 g, 15.92 mmol, 1.23 mL) at −40° C. Then 2-(2-iodoethoxy)ethanol (2.29 g, 10.61 mmol) was added and the mixture was stirred at −20° C. for 3h. The reaction mixture was purified via silica gel chromatography (PE:EA=1:1) to provide (23E,25E,27E,28E,34R,35S,36R,37R,39S,41S,44S,45R,46R,55R)-44-[(1R)-2-[(1S,3R,4R)-3,4-dimethoxycyclohexyl]-1-methyl-ethyl]-55-hydroxy-43-[2-(2-iodoethoxy)ethoxy]-45,46-dimethoxy-34,35,36,37,47,48-hexamethyl-63,64-dioxa-56-azatricyclohexatriaconta-23,25,27(47),28(48)-tetraene-49,50,51,52,53-pentone (0.3 g, 50% yield) as a white solid.

Step 2: (24E,26E,28E,29E,35R,36S,37R,38R,40S,42S,45S,46R,47R,56R)-45-[(1R)-2-[(1S,3R,4R)-3,4-dimethoxycyclohexyl]-1-methyl-ethyl]-56-hydroxy-46,47-dimethoxy-35,36,37,38,48,49-hexamethyl-44-[2-[2-(methylamino)ethoxy]ethoxy]-65,66-dioxa-58-azatricyclohexatriaconta-24,26,28(48),29(49)-tetraene-50,51,52,53,54-pentone (I-90). A solution of (23E,25E,27E,28E,34R,35S,36R,37R,39S,41S,44S,45R,46R,55R)-44-[(1R)-2-[(1S,3R,4R)-3,4-dimethoxycyclohexyl]-1-methyl-ethyl]-55-hydroxy-43-[2-(2-iodoethoxy)ethoxy]-45,46-dimethoxy-34,35,36,37,47,48-hexamethyl-63,64-dioxa-56-azatricyclohexatriaconta-23,25,27(47),28(48)-tetraene-49,50,51,52,53-pentone (0.38 g, 0.34 mmol), methanamine (0.105 g, 3.37 mmol, 0.117 mL) and N-ethyl-N-isopropyl-propan-2-amine (0.436 g, 3.37 mmol, 0.588 mL) in DCM (8 mL) was stirred for 24 h at 22° C. The reaction was diluted with DCM (10 mL) and washed with saturated NH₄Cl (10 mL×3), water (10 mL×3) and brine (10 mL×3), dried over anhydrous sodium sulfate, filtered and concentrated. The residue was purified via reverse-phase chromatography eluting with 50% CH₃CN in water to provide I-90 (55 mg, 16% yield) as a white solid.

### Example 38: Synthesis of (23E,25E,27E,28E,32R,33S,34R,35R,37S,39S,41R,42S,44R,45R,54R)-44,54-dihydroxy-42-[(1R)-2-[(1S,3R,4R)-4-hydroxy-3-methoxy-cyclohexyl]-1-methyl-ethyl]-45-methoxy-32,33,34,35,46,47-hexamethyl-41-[2-(oxetan-3-yloxy)ethoxy]-65,66-dioxa-55-azatricyclohexatriaconta-23,25,27(46),28(47)-tetraene-48,49,50,51,52-pentone (I-88), (1R,9S,12S,15R,16E,18R,19R,21R,23S,24E,26E,28E,30S,32S,35R)-1,18-dihydroxy-12-[(1R)-2-[(1S,3R,4R)-4-hydroxy-3-methoxy-cyclohexyl]-1-methyl-ethyl]-19-methoxy-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone (I-72) and (1R,9S,12S,15R,16E,18R,19R,21R,23S,24E,26E,28E,30R,32S,35R)-1,18-dihydroxy-12-[(1R)-2-[(1S,3R,4R)-4-hydroxy-3-methoxy-cyclohexyl]-1-methyl-ethyl]-19-methoxy-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone (I-71)

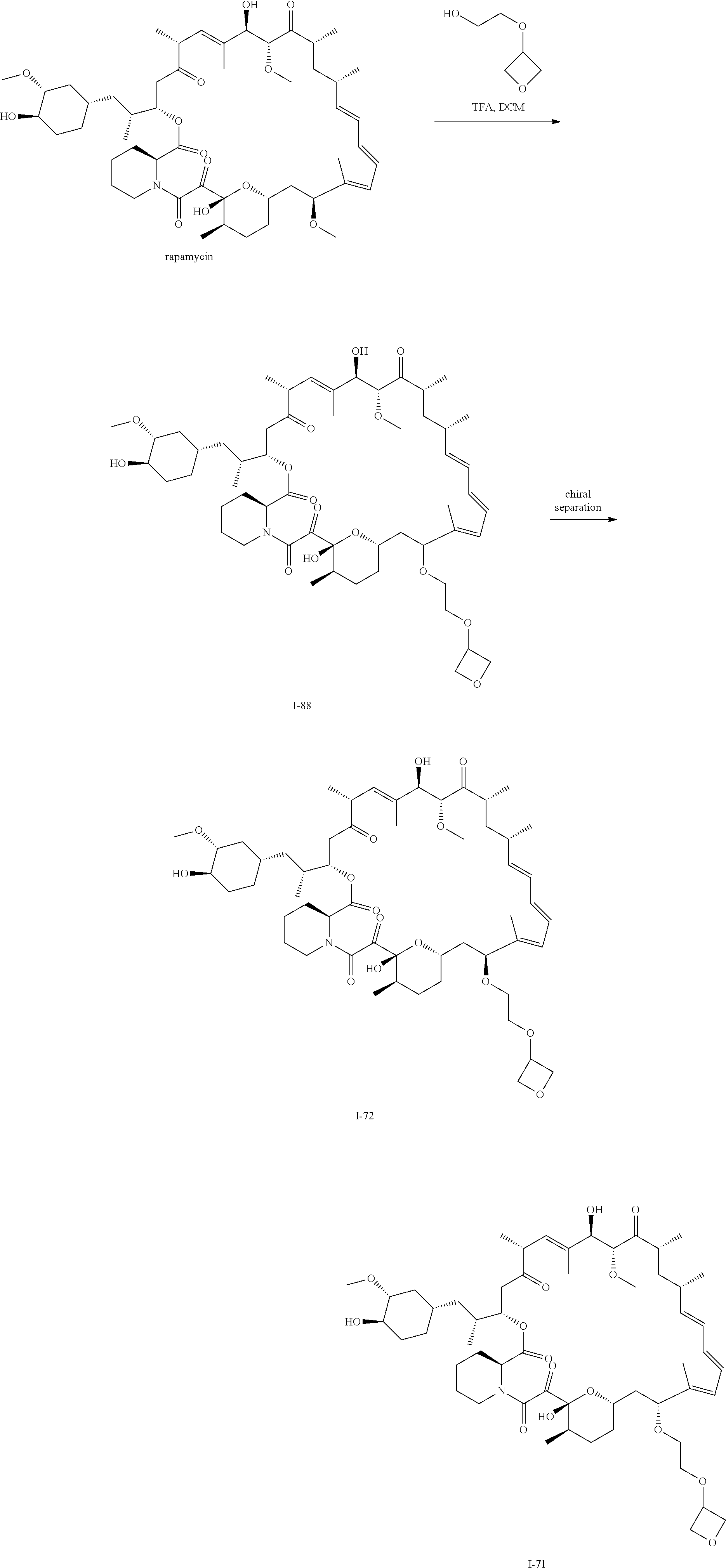

Step 1: (23E,25E,27E,28E,32R,33S,34R,35R,37S,39S,41R,42S,44R,45R,54R)-44,54-dihydroxy-42-[(1R)-2-[(1S,3R,4R)-4-hydroxy-3-methoxy-cyclohexyl]-1-methyl-ethyl]-45-methoxy-32,33,34,35,46,47-hexamethyl-41-[2-(oxetan-3-yloxy)ethoxy]-65,66-dioxa-55-azatricyclohexatriaconta-23,25,27(46),28(47)-tetraene-48,49,50,51,52-pentone (I-88). To a solution of rapamycin (0.5 g, 0.547 mmol) in DCM (10 mL) under nitrogen was added TFA (1.87 g, 16.41 mmol) at −40° C. followed by 2-(oxetan-3-yloxy) ethanol (1.29 g, 10.94 mmol) and the mixture was stirred at −20° C. for 2h. The reaction mixture was quenched by adding saturated aqueous NaHCO₃ (20 mL) and extracted with DCM (30 mL) at 0° C. The organic layer was washed with water (20 mL) and brine (20 mL), dried over anhydrous sodium sulfate, filtered and concentrated. The residue was purified by reverse phase column chromatography eluting with 80% CH₃CN in water to provide I-88 (120 mg, 22% yield) as a white solid. ESI-MS (EI⁺, m/z): 1022.7 [M+Na]⁺. ¹H NMR (400 MHz, CDCl₃) δ 6.42-5.88 (m, 4H), 5.58-5.08 (m, 4H), 4.83-4.54 (m, 5H), 4.35-3.93 (m, 2H), 3.91-3.68 (m, 3H), 3.53-3.21 (m, 13H), 2.99-2.41 (m, 5H), 2.38-1.87 (m, 7H), 1.85-1.58 (m, 13H), 1.55-1.17 (m, 11H), 1.16-0.82 (m, 17H), 0.80-0.63 (m, 1H).

Step 2: (1R,9S,12S,15R,16E,18R,19R,21R,23S,24E,26E,28E,30S,32S,35R)-1,18-dihydroxy-12-[(1R)-2-[(1S,3R,4R)-4-hydroxy-3-methoxy-cyclohexyl]-1-methyl-ethyl]-19-methoxy-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone (I-72) and (1R,9S,12S,15R,16E,18R,19R,21R,23S,24E,26E,28E,30R,32S,35R)-1,18-dihydroxy-12-[(1R)-2-[(1S,3R,4R)-4-hydroxy-3-methoxy-cyclohexyl]-1-methyl-ethyl]-19-methoxy-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone (I-71). 200 mg of I-88 was separated via chiral preparatory HPLC and then purified via silica gel chromatography to obtain I-72 (31 mg, 16% yield) as a white solid and I-71 (18 mg, 9% yield) as a white solid.

Chiral analysis method:

Column: CHIRALPAK IC-3 (IC30CE-NJ008)

Column size: 0.46 cm I.D.×25 cm L

Injection: 10 μL

Mobile phase: Hexane/EtOH=50/50 (V/V)

Flow rate: 1.0 mL/min

Wavelength: UV 254 nm

Temperature: 35° C.

HPLC equipment: Shimadzu LC-20AT

I-72: ESI-MS (EI⁺, m/z): 1022.3 [M+Na]⁺. ¹H NMR (400 MHz, CDCl₃) δ 6.37-5.77 (m, 4H), 5.51-4.99 (m, 4H), 4.77-4.45 (m, 5H), 4.12 (dd, J=13.5, 6.1 Hz, 1H), 3.84-3.58 (m, 3H), 3.54-2.99 (m, 15H), 2.93-2.48 (m, 5H), 2.34-1.63 (m, 14H), 1.49-1.10 (m, 14H), 1.08-0.71 (m, 19H), 0.59 (dt, J=16.8, 8.4 Hz, 1H).
I-71: ESI-MS (EI⁺, m/z): 1022.3 [M+Na]⁺. ¹H NMR (400 MHz, CDCl₃) δ 6.44-5.86 (m, 4H), 5.66-4.99 (m, 4H), 4.77-4.46 (m, 5H), 4.25-3.59 (m, 5H), 3.55-3.02 (m, 15H), 2.96-1.83 (m, 13H), 1.81-1.59 (m, 10H), 1.46-1.10 (m, 10H), 1.08-0.48 (m, 19H).

### Example 39: Synthesis of (1R,9S,12S,15R,16E,18R,19R,21R,23S,24E,26E,28E,32S,35R)-12-[(1R)-2-[(1S,3R,4R)-4-dimethylphosphoryloxy-3-methoxy-cyclohexyl]-1-methyl-ethyl]-1,18-dihydroxy-19-methoxy-15,17,21,23,29,35-hexamethyl-30-[2-(oxetan-3-yloxy)ethoxy]-11,36-dioxa-4-azatricyclo[30.3.1.04,9]hexatriaconta-16,24,26,28-tetraene-2,3,10,14,20-pentone (I-89)

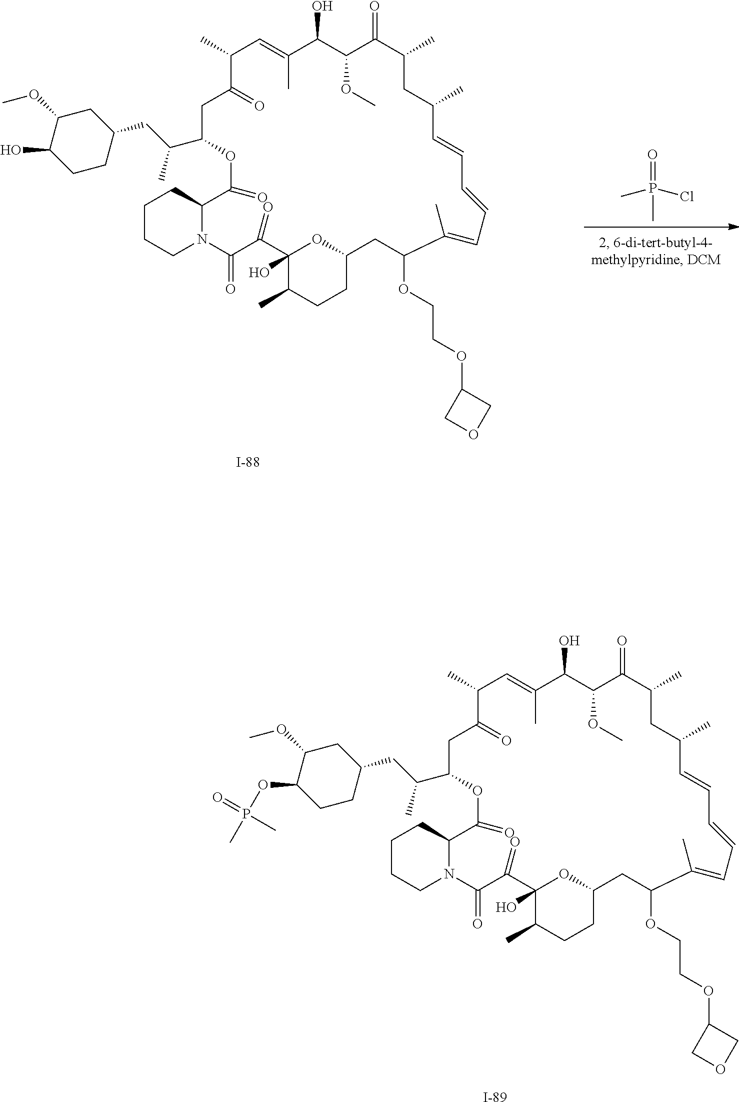

To a solution of (23E,25E,27E,28E,32R,33S,34R,35R,37S,39S,41R,42S,44R,45R,54R)-44,54-dihydroxy-42-[(1R)-2-[(1S,3R,4R)-4-hydroxy-3-methoxy-cyclohexyl]-1-methyl-ethyl]-45-methoxy-32,33,34,35,46,47-hexamethyl-41-[2-(oxetan-3-yloxy)ethoxy]-65,66-dioxa-55-azatricyclohexatriaconta-23,25,27(46),28(47)-tetraene-48,49,50,51,52-pentone (I-88) (0.290 g, 0.290 mmol) in DCM (7 mL) was added 2,6-di-tert-butyl-4-methylpyridine (0.447 g, 2.17 mmol) at 0° C., followed by dimethylphosphinic chloride (0.16 g, 1.45 mmol) in DCM (2 mL). The resulting solution was stirred at 0° C. for 7 hr, then diluted with DCM, washed with saturated NaHCO₃ (30 mL), 0.5N HCl aqueous solution, water (30 mL), brine (50 mL), then the organic layer was dried over Na₂SO₄, filtrated and concentrated. The residue was purified via silica gel chromatography (MeOH in PE:EA:DCM (3:3:10) from 0 to 15%), then by reverse phase chromatography (60% CH₃CN in water) to provide I-89 (0.13 g, 42% yield) as a white solid.

### Example 40: Synthesis of (3S,6R,7E,9R,10R,12R,14S,15E,17E,19E,23S,26R,27R,34aS)-27-hydroxy-3-((R)-1-((1S,3R,4R)-4-(2-hydroxyethoxy)-3-methoxycyclohexyl)propan-2-yl)-9,10-dimethoxy-6,8,12,14,20,26-hexamethyl-21-(2-(oxetan-3-yloxy)ethoxy)-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (I-87)

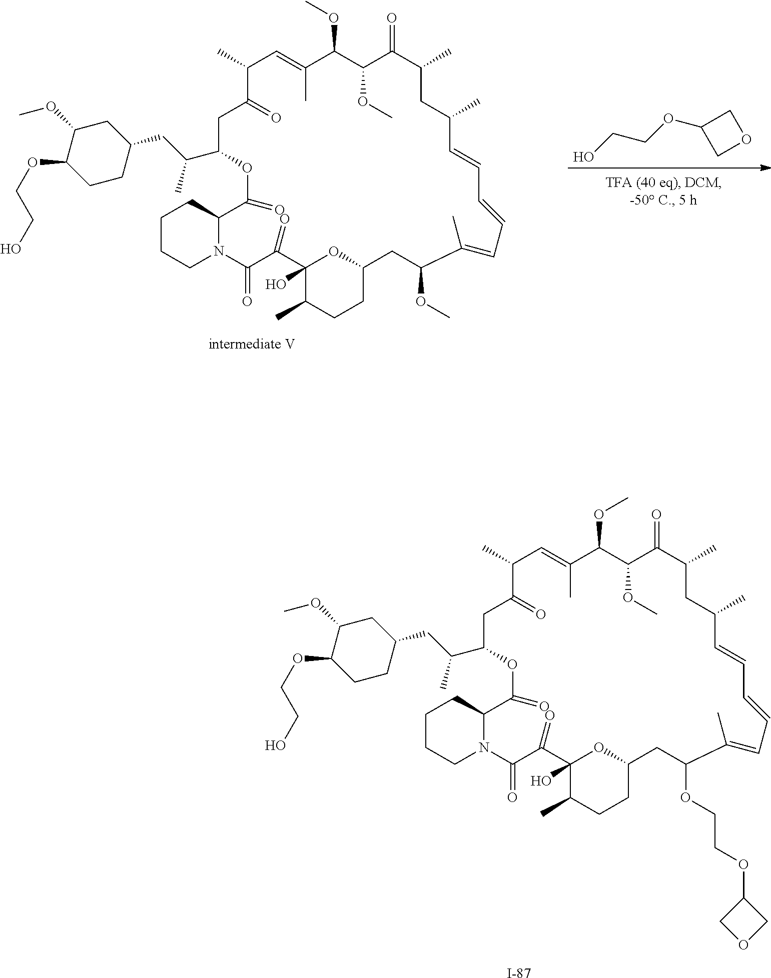

Step 1: (3S,6R,7E,9R,10R,12R,14S,15E,17E,19E,23S,26R,27R,34aS)-27-hydroxy-3-((R)-1-((1S,3R,4R)-4-(2-hydroxyethoxy)-3-methoxycyclohexyl)propan-2-yl)-9,10-dimethoxy-6,8,12,14,20,26-hexamethyl-21-(2-(oxetan-3-yloxy)ethoxy)-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (I-87). To a solution of Intermediate V (0.66 g, 0.679 mmol) in DCM (10 mL) was added TFA (3.1 g, 27.15 mmol, 2.09 mL) at −50° C. After 10 minutes 2-(oxetan-3-yloxy)ethanol (2.41 g, 20.37 mmol) in DCM (0.5 mL) was added and the mixture stirred at 0° C. for 5 h. DCM and aqueous NaHCO₃ solution was added and the organic layer washed with water and brine, dried over Na₂SO₄, filtered and concentrated. The residue was purified via reverse-phase chromatography to provide I-87 (129.4 mg, 18% yield) as a white solid. ESI-MS (EI⁺, m/z): 1080.6 [M+Na]⁺. ¹H NMR (500 MHz, CDCl₃) δ 6.46-5.93 (m, 4H), 5.65-5.01 (m, 4H), 4.82-4.15 (m, 4H), 3.92-3.54 (m, 9H), 3.51-3.07 (m, 16H), 2.95-2.48 (m, 3H), 2.37-1.83 (m, 6H), 1.82-1.46 (m, 19H), 1.44-1.21 (m, 7H), 1.17-0.81 (m, 18H), 0.74 (d, J=11.9 Hz, 1H).

### Example 41: Synthesis of (1R,9S,12S,15R,16E,18R,19R,21R,23S,24E,26E,28E,32S,35R)-12-[(1R)-2-[(1S,3R,4R)-4-dimethylphosphoryloxy-3-methoxy-cyclohexyl]-1-methyl-ethyl]-1,18-dihydroxy-19-methoxy-15,17,21,23,29,35-hexamethyl-30-(2-tetrahydropyran-4-yloxyethoxy)-11,36-dioxa-4-azatricyclo[30.3.1.0^{4,9}]hexatriaconta-16,24,26,28-tetraene-2,3,10,14,20-pentone (I-86)

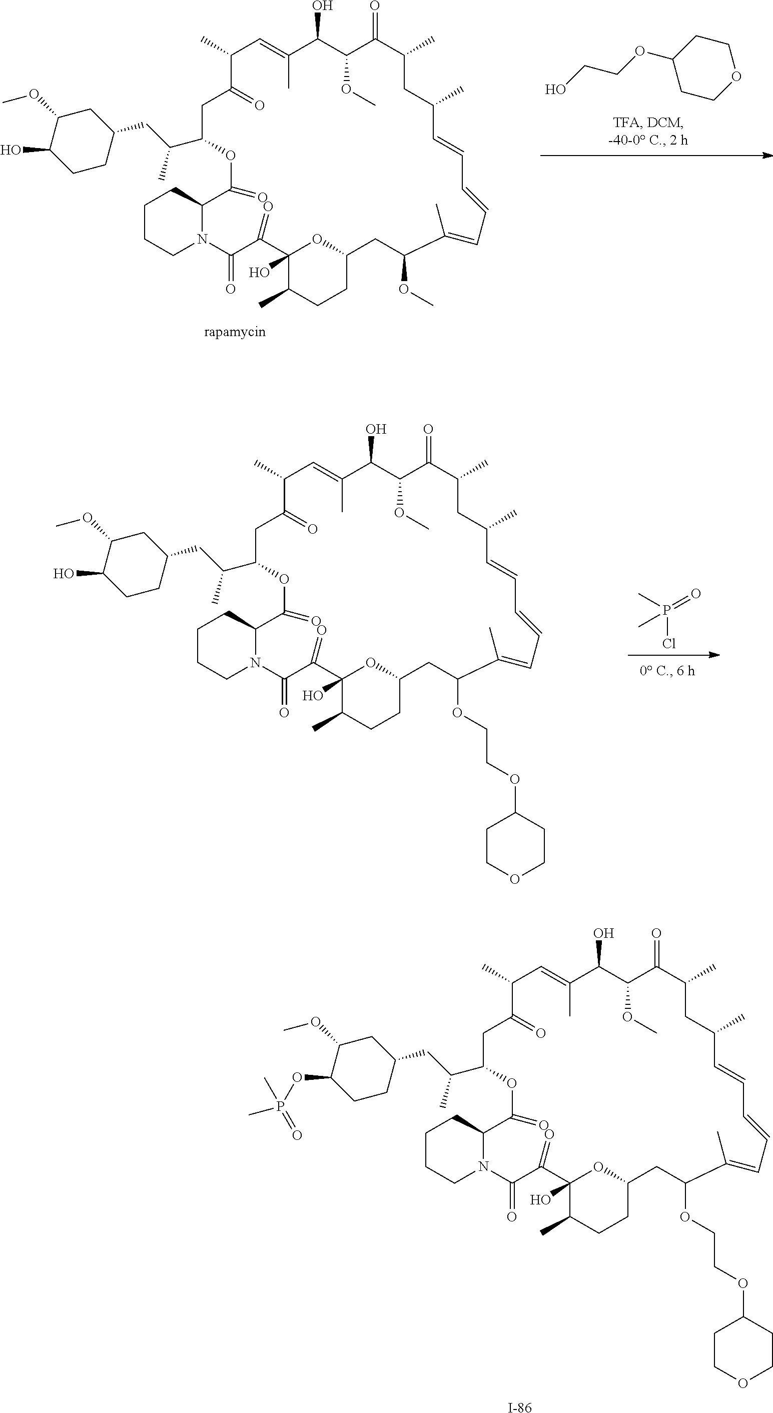

Step 1: (25E,27E,29E,30E,34R,35S,36R,37R,40S,42S,45S,46R,47R,56R)-46,56-dihydroxy-45-[(1R)-2-[(1S,3R,4R)-4-hydroxy-3-methoxy-cyclohexyl]-1-methyl-ethyl]-47-methoxy-34,35,36,37,48,49-hexamethyl-44-(2-tetrahydropyran-4-yloxyethoxy)-67,68-dioxa-57-azatricyclohexatriaconta-25,27,29(48),30(49)-tetraene-50,51,52,53,54-pentone. To a solution of rapamycin (0.5 g, 0.547 mmol) and 2-tetrahydropyran-4-yloxyethanol (2.4 g, 16.41 mmol) in DCM (15 mL) was added 2,2,2-trifluoroacetic acid (2.49 g, 21.88 mmol) at 0° C. under N₂. The reaction mixture was stirred at 0° C. for 2 h then washed with cold saturated NaHCO₃ solution (10 mL), water (10 mL×3) and brine (10 mL×3), dried over anhydrous sodium sulfate, filtered and concentrated, The residue was purified via reverse phase column chromatography eluting with 70% CH₃CN in water to provide (25E,27E,29E,30E,34R,35S,36R,37R,40S,42S,45S,46R,47R,56R)-46,56-dihydroxy-45-[(1R)-2-[(1S,3R,4R)-4-hydroxy-3-methoxy-cyclohexyl]-1-methyl-ethyl]-47-methoxy-34,35,36,37,48,49-hexamethyl-44-(2-tetrahydropyran-4-yloxyethoxy)-67,68-dioxa-57-azatricyclohexatriaconta-25,27,29(48),30(49)-tetraene-50,51,52,53,54-pentone (175 mg, 31% yield) as a white solid. ESI-MS (EI+, m/z):1050.6 [M+Na]⁺.

Step 2: (1R,9S,12S,15R,16E,18R,19R,21R,23S,24E,26E,28E,32S,35R)-12-[(1R)-2-[(1S,3R,4R)-4-dimethylphosphoryloxy-3-methoxy-cyclohexyl]-1-methyl-ethyl]-1,18-dihydroxy-19-methoxy-15,17,21,23,29,35-hexamethyl-30-(2-tetrahydropyran-4-yloxyethoxy)-11,36-dioxa-4-azatricyclo[30.3.1.0^{4,9}]hexatriaconta-16,24,26,28-tetraene-2,3,10,14,20-pentone (I-86). To a solution of (1R,9S,12S,15R,16E,18R,19R,21R,23S,24E,26E,28E,32S,35R)-1,18-dihydroxy-12-[(1R)-2-[(1S,3R,4R)-4-hydroxy-3-methoxy-cyclohexyl]-1-methyl-ethyl]-19-methoxy-15,17,21,23,29,35-hexamethyl-30-(2-tetrahydropyran-4-yloxyethoxy)-11,36-dioxa-4-azatricyclo[30.3.1.0^{4,9}]hexatriaconta-16,24,26,28-tetraene-2,3,10,14,20-pentone (0.25 g, 0.24 mmol) in DCM (9 mL) was added 2,6-di-tert-butyl-4-methylpyridine (0.37 g, 1.82 mmol) and [chloro(methyl)phosphoryl]methane (0.137 g, 1.22 mmol) in DCM (3 mL) at 0° C. under N₂. The mixture was stirred at 0° C. for 6 h then diluted with DCM (30 mL), washed with saturated NaHCO₃(30 mL), 0.5N HCl aqueous solution, water (30 mL), brine (50 mL), then, the organic layer was dried over Na₂SO₄, filtered and concentrated in vacuo and the crude was purified via silica gel chromatography (MeOH in PE:EA:DCM (3:3:10) from 0 to 15%) to afford I-86 (130 mg, 48% yield) as a white solid. ESI-MS (EI⁺, m/z):1126.4 [M+Na]⁺. ¹H NMR (500 MHz, CDCl₃) δ 6.53-5.81 (m, 4H), 5.6-5.12 (m, 4H), 4.33-3.70 (m, 7H), 3.65-2.90 (m, 16H), 2.84-1.97 (m, 8H), 1.95-1.17 (m, 36H), 1.14-0.63 (m, 19H).

### Example 42: Synthesis of (24E,26E,28E,29E,34R,35S,36R,37R,39S,41S,44S,46R,47R,56R)-43-[[(2S)-1,4-dioxan-2-yl]methoxy]-46,56-dihydroxy-44-[(1R)-2-[(1S,3R,4R)-4-(2-hydroxyethoxy)-3-methoxy-cyclohexyl]-1-methyl-ethyl]-47-methoxy-34,35,36,37,48,49-hexamethyl-68,69-dioxa-57-azatricyclohexatriaconta-24,26,28(48),29(49)-tetraene-50,51,52,53,54-pentone (I-110), (1R,9S,12S,15R,16E,18R,19R,21R,23S,24E,26E,28E,30S,32S,35R)-30-[[(2S)-1,4-dioxan-2-yl]methoxy]-1,18-dihydroxy-12-[(1R)-2-[(1S,3R,4R)-4-(2-hydroxyethoxy)-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (I-85) and (1R,9S,12S,15R,16E,18R,19R,21R,23S,24E,26E,28E,30R,32S,35R)-30-[[(2S)-1,4-dioxan-2-yl]methoxy]-1,18-dihydroxy-12-[(1R)-2-[(1S,3R,4R)-4-(2-hydroxyethoxy)-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (I-84)

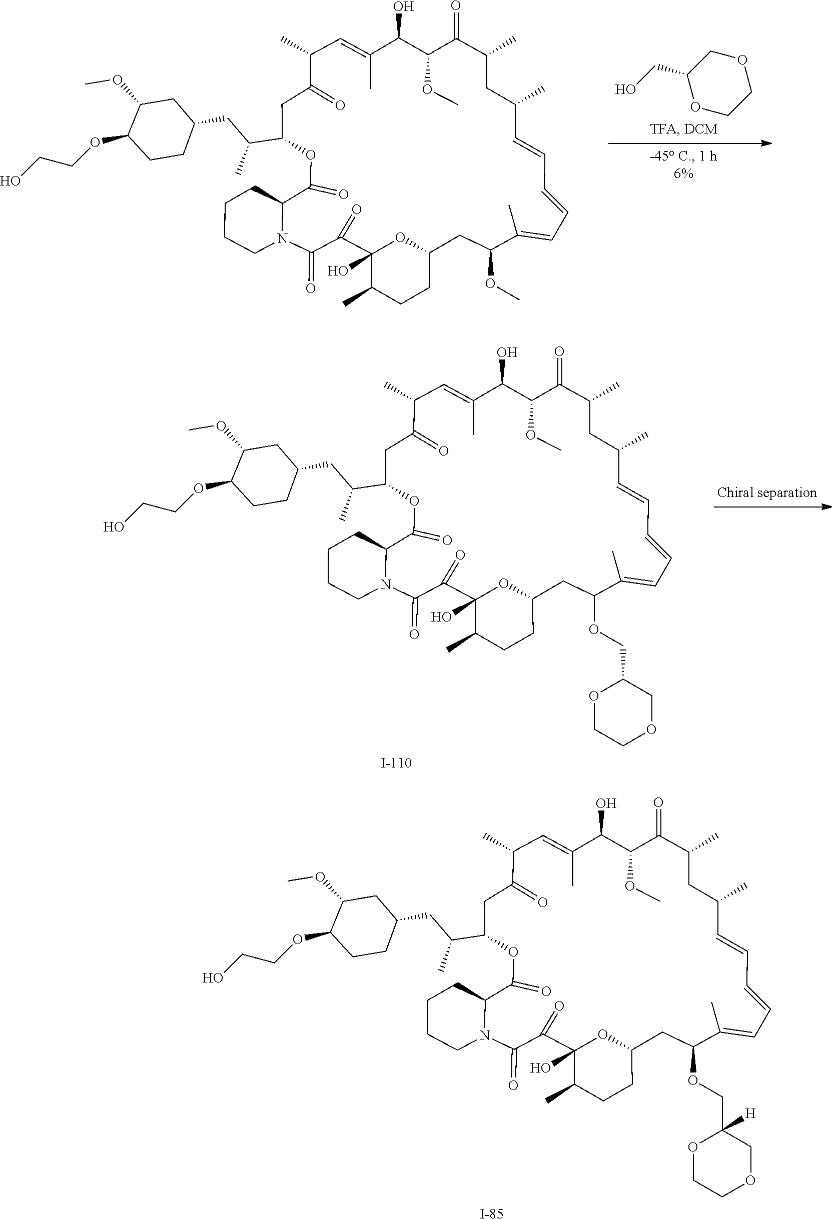

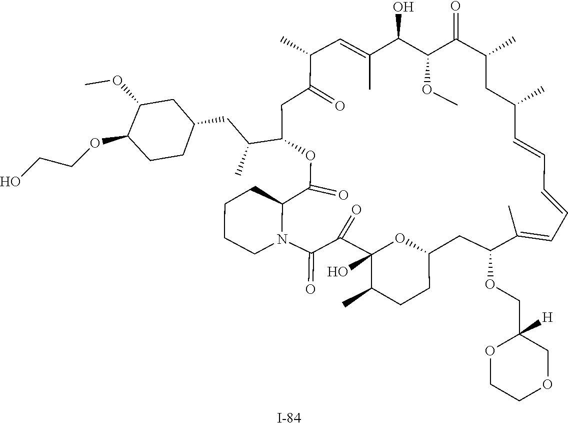

Step 1: (24E,26E,28E,29E,34R,35S,36R,37R,39S,41S,44S,46R,47R,56R)-43-[[(2S)-1,4-dioxan-2-yl]methoxy]-46,56-dihydroxy-44-[(1R)-2-[(1S,3R,4R)-4-(2-hydroxyethoxy)-3-methoxy-cyclohexyl]-1-methyl-ethyl]-47-methoxy-34,35,36,37,48,49-hexamethyl-68,69-dioxa-57-azatricyclohexatriaconta-24,26,28(48),29(49)-tetraene-50,51,52,53,54-pentone (I-110). To a solution of everolimus (1 g, 1.04 mmol) in DCM (60 mL) was added 2,2,2-trifluoroacetic acid (2.38 g, 20.88 mmol, 1.61 mL) dropwise at −55° C. under N₂. The reaction mixture was stirred for 10 min at −45° C. then [(2R)-1,4-dioxan-2-yl]methanol (0.493 g, 4.17 mmol) in DCM was added and the reaction stirred for 1 h at −20° C. The mixture was poured into saturated aqueous NaHCO₃ (80 mL) at 0° C. and extracted with DCM (80 mL). The organic layer was washed with water (80 mL) and brine (80 mL), dried over anhydrous sodium sulfate, filtered and concentrated. The residue was purified by reverse phase chromatography then re-purified via silica gel chromatography (100% EA) to provide I-110 (65 mg, 6% yield) as a white solid. ESI-MS (EI⁺, m/z): 1066.4 [M+Na]⁺. ¹H NMR (400 MHz, CDCl₃) δ 6.39-5.92 (m, 4H), 5.56-4.81 (m, 5H), 4.26-3.98 (m, 2H), 3.84-3.68 (m, 9H), 3.62-3.53 (m, 3H), 3.48-3.04 (m, 15H), 2.87-2.55 (m, 4H), 2.35-1.83 (m, 7H), 1.79-1.38 (m, 27H), 1.34-1.22 (m, 7H), 1.18-0.79 (m, 19H), 0.76-0.67 (m, 1H).

Step 2: (1R,9S,12S,15R,16E,18R,19R,21R,23S,24E,26E,28E,30S,32S,35R)-30-[[(2S)-1,4-dioxan-2-yl]methoxy]-1,18-dihydroxy-12-[(1R)-2-[(1S,3R,4R)-4-(2-hydroxyethoxy)-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (I-85) and (1R,9S,12S,15R,16E,18R,19R,21R,23S,24E,26E,28E,30R,32S,35R)-30-[[(2S)-1,4-dioxan-2-yl]methoxy]-1,18-dihydroxy-12-[(1R)-2-[(1S,3R,4R)-4-(2-hydroxyethoxy)-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (I-84). 110 mg of (I-110 was separated via chiral preparatory HPLC and then purified via silica gel chromatography to provide I-85 (32.2 mg, 29% yield) as a white solid and I-84 (12 mg 11% yield) as a white solid.

Chiral analysis method:

Column: CHIRALPAK IC (IC00CD-TB016)

Column size: 0.46 cm I.D.×15 cm L

Injection: 20 μL

Mobile phase: Hexane/EtOH=50/50 (V/V)

Flow rate: 1.0 mL/min

Wavelength: UV 254 nm

Temperature: 35° C.

HPLC equipment: Shimadzu LC-20AT

I-85: ESI-MS (EI⁺, m/z): 1066.3 [M+Na]⁺. ¹H NMR (400 MHz, CDCl₃) δ 6.42-5.82 (m, 4H), 5.59-5.07 (m, 4H), 4.81 (s, 1H), 4.17 (d, J=6.1 Hz, 1H), 3.89-3.53 (m, 15H), 3.47-3.01 (m, 16H), 2.90-2.52 (m, 3H), 2.41-1.85 (m, 8H), 1.82-1.42 (m, 8H), 1.39-1.18 (m, 10H), 1.15-0.61 (m, 19H).
I-84: ESI-MS (EI⁺, m/z): 1066.3 [M+Na]⁺. ¹H NMR (400 MHz, CDCl₃) δ 6.42-5.86 (m, 4H), 5.59-5.07 (m, 4H), 4.30-3.95 (m, 3H), 3.87-3.03 (m, 28H), 2.97-1.71 (m, 21H), 1.52-1.17 (m, 14H), 1.13-0.64 (m, 19H).

### Example 43: Synthesis of (1R,9S,12S,15R,16E,18R,19R,21R,23S,24E,26E,28E,32S,35R)-1-hydroxy-18,19-dimethoxy-12-[(1R)-2-[(1S,3R,4R)-3-methoxy-4-(2-methoxyethoxy)cyclohexyl]-1-methyl-ethyl]-15,17,21,23,29,35-hexamethyl-30-[2-[2-(oxetan-3-yloxy)ethoxy]ethoxy]-11,36-dioxa-4-azatricyclo[30.3.1.0^{4,9}]hexatriaconta-16,24,26,28-tetraene-2,3,10,14,20-pentone (I-81), (1R,9S,12S,15R,16E,18R,19R,21R,23S,24E,26E,28E,30S,32S,35R)-1-hydroxy-18,19-dimethoxy-12-[(1R)-2-[(1S,3R,4R)-3-methoxy-4-(2-methoxyethoxy)cyclohexyl]-1-methyl-ethyl]-15,17,21,23,29,35-hexamethyl-30-[2-[2-(oxetan-3-yloxy)ethoxy]ethoxy]-11,36-dioxa-4-azatricyclo[30.3.1.0^{4,9}]hexatriaconta-16,24,26,28-tetraene-2,3,10,14,20-pentone (I-69)

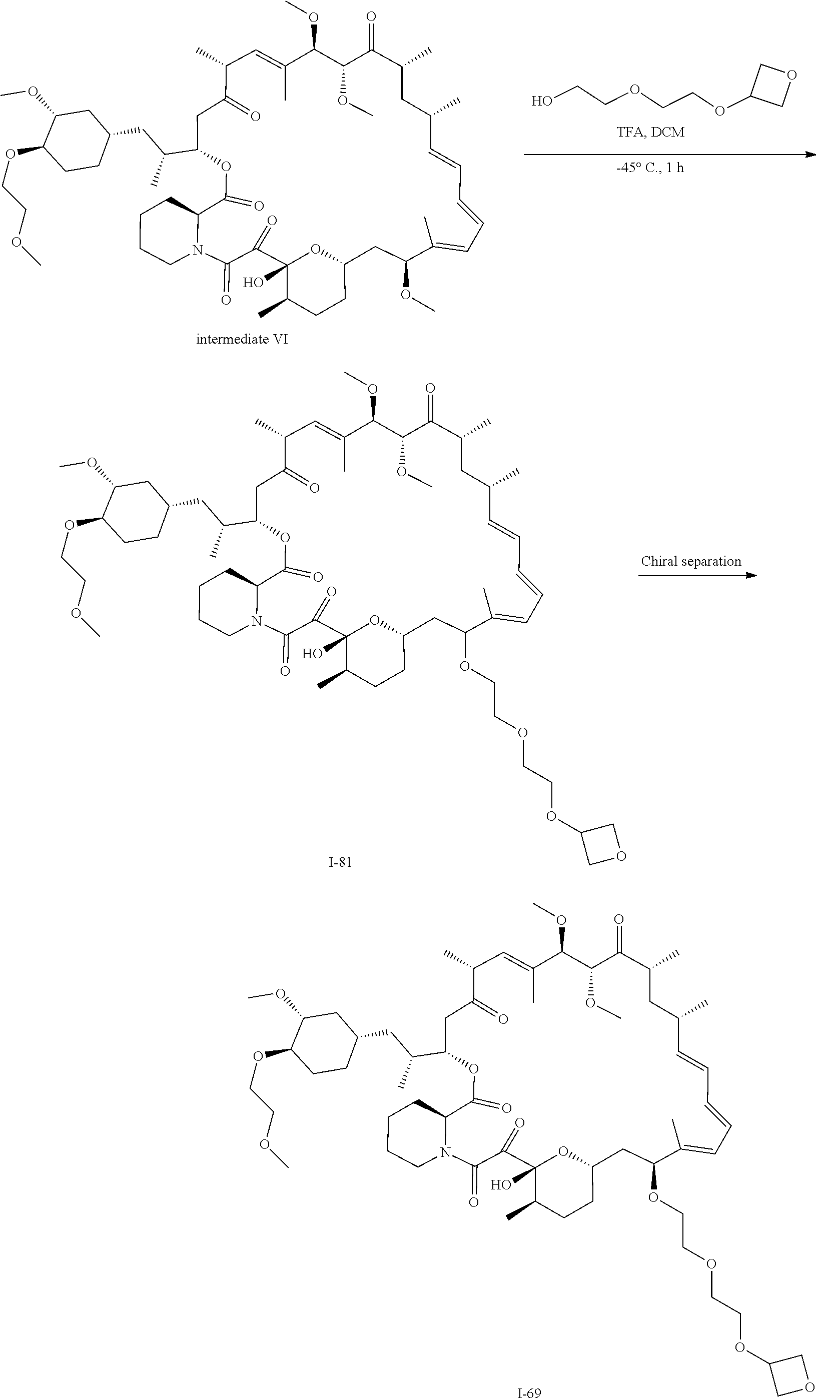

Step 1: (1R,9S,12S,15R,16E,18R,19R,21R,23S,24E,26E,28E,32S,35R)-1-hydroxy-18,19-dimethoxy-12-[(1R)-2-[(1S,3R,4R)-3-methoxy-4-(2-methoxyethoxy)cyclohexyl]-1-methyl-ethyl]-15,17,21,23,29,35-hexamethyl-30-[2-[2-(oxetan-3-yloxy)ethoxy]ethoxy]-11,36-dioxa-4-azatricyclo[30.3.1.0^{4,9}]hexatriaconta-16,24,26,28-tetraene-2,3,10,14,20-pentone (I-81). To a solution of Intermediate VI (0.5 g, 0.507 mmol) in DCM (20 mL) was added 2,2,2-trifluoroacetic acid (1.16 g, 10.14 mmol, 0.78 mL) dropwise at −55° C. under N₂. The reaction was stirred for 10 min at −45° C. then 2-[2-(oxetan-3-yloxy) ethoxy] ethanol (1.64 g, 10.14 mmol) in DCM was added at the same temperature. The mixture was warmed to 0° C. over 2 h. The reaction was poured into saturated aqueous NaHCO₃ (40 mL) at 0° C. and extracted with DCM (40 mL). The organic layer was washed with water (40 mL) and brine (40 mL), dried over anhydrous sodium sulfate, filtered and concentrated. The residue was purified via silica gel chromatography (100% EA) and re-purified via reverse phase chromatography eluting with 60% CH₃CN in water to give I-81 (0.03 g, 5% yield) as a white solid. ESI-MS (EI⁺, m/z): 1138.7 [M+Na]⁺. ¹H NMR (400 MHz, CDCl₃) δ 6.45-6.00 (m, 4H), 5.54-5.08 (m, 4H), 4.78-4.57 (m, 5H), 4.45-4.30 (m, 1H), 3.88-3.79 (m, 4H), 3.70-3.50 (m, 9H), 3.47-3.42 (m, 4H), 3.38-3.30 (m, 5H), 3.28-3.23 (m, 3H), 3.22-3.03 (m, 5H), 2.75-2.50 (m, 2H), 2.31-1.84 (m, 6H), 1.76-1.48 (m, 18H), 1.53-1.21 (m, 10H), 1.18-1.04 (m, 11H), 0.98-0.83 (m, 8H), 0.80-0.67 (m, 2H).

Step 2: (1R,9S,12S,15R,16E,18R,19R,21R,23S,24E,26E,28E,30S,32S,35R)-1-hydroxy-18,19-dimethoxy-12-[(1R)-2-[(1S,3R,4R)-3-methoxy-4-(2-methoxyethoxy)cyclohexyl]-1-methyl-ethyl]-15,17,21,23,29,35-hexamethyl-30-[2-[2-(oxetan-3-yloxy)ethoxy]ethoxy]-11,36-dioxa-4-azatricyclo[30.3.1.0^{4,9}]hexatriaconta-16,24,26,28-tetraene-2,3,10,14,20-pentone (I-69). 100 mg of I-81 was separated via chiral preparatory HPLC and then via silica gel chromatography to obtain I-69 (25 mg, 25% yield) as a white solid.

Chiral analysis method:

Column: CHIRALPAK IC (IC00CE-BN011)

Column size: 0.46 cm I.D.×25 cm L

Injection: 50 μL

Mobile phase: Hexane/EtOH=50/50 (V/V)

Flow rate: 1.0 mL/min

Wavelength: UV 254 nm

Temperature: 35° C.

HPLC equipment: Shimadzu LC-20AT

I-69: ESI-MS (EI⁺, m/z): 1138.3 [M+Na]⁺. ¹H NMR (400 MHz, CDCl₃) δ 6.42-5.73 (m, 4H), 5.72-4.98 (m, 4H), 4.72-4.47 (m, 5H), 3.94-2.92 (m, 32H), 2.90-2.39 (m, 3H), 2.33-1.49 (m, 17H), 1.47-1.13 (m, 12H), 1.07-0.57 (m, 20H).

### Example 44: Synthesis of (1R,9S,12S,15R,16E,18R,19R,21R,23S,24E,26E,28E,32S,35R)-1-hydroxy-12-[(1R)-2-[(1S,3R,4R)-4-(2-hydroxyethoxy)-3-methoxy-cyclohexyl]-1-methyl-ethyl]-18,19-dimethoxy-15,17,21,23,29,35-hexamethyl-30-[2-[2-(oxetan-3-yloxy)ethoxy]ethoxy]-11,36-dioxa-4-azatricyclo[30.3.1.0^{4,9}]hexatriaconta-16,24,26,28-tetraene-2,3,10,14,20-pentone (I-80) and (1R,9S,12S,15R,16E,18R,19R,21R,23S,24E,26E,28E,30S,32S,35R)-1-hydroxy-12-[(1R)-2-[(1S,3R,4R)-4-(2-hydroxyethoxy)-3-methoxy-cyclohexyl]-1-methyl-ethyl]-18,19-dimethoxy-15,17,21,23,29,35-hexamethyl-30-[2-[2-(oxetan-3-yloxy)ethoxy]ethoxy]-11,36-dioxa-4-azatricyclo[30.3.1.0^{4,9}]hexatriaconta-16,24,26,28-tetraene-2,3,10,14,20-pentone (I-62)

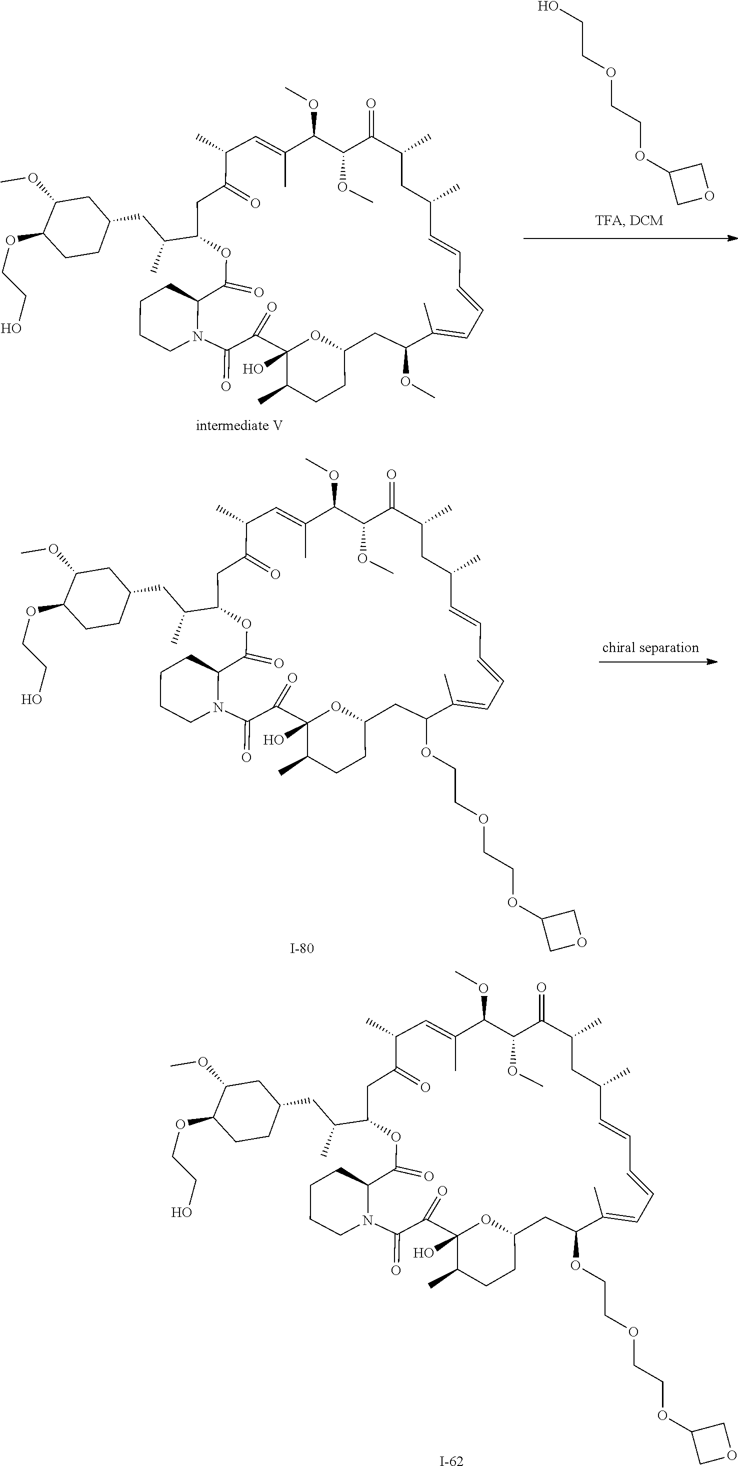

Step 1: (1R,9S,12S,15R,16E,18R,19R,21R,23S,24E,26E,28E,32S,35R)-1-hydroxy-12-[(1R)-2-[(1S,3R,4R)-4-(2-hydroxyethoxy)-3-methoxy-cyclohexyl]-1-methyl-ethyl]-18,19-dimethoxy-15,17,21,23,29,35-hexamethyl-30-[2-[2-(oxetan-3-yloxy)ethoxy]ethoxy]-11,36-dioxa-4-azatricyclo[30.3.1.0^{4,9}]hexatriaconta-16,24,26,28-tetraene-2,3,10,14,20-pentone (I-80). To a solution of Intermediate V (0.6 g, 0.62 mmol) in DCM (15 mL) was added TFA (2.11 g, 18.51 mmol, 1.43 mL) at −40° C. under N₂, 2-[2-(oxetan-3-yloxy) ethoxy] ethanol (2 g, 12.34 mmol) was then added and the mixture was stirred at −20° C. for 2h. The reaction was poured into a solution of saturated NaHCO₃(aq) solution (20 mL) at 0° C. and extracted with DCM (20 mL). The organic layer was washed with water (20 mL) and brine (20 mL), dried over anhydrous sodium sulfate, filtered and concentrated. The residue was purified by reverse phase chromatography to obtain I-80 (50 mg, 7% yield) as a light yellow solid. ESI-MS (EI⁺, m/z): 1124.7 [M+Na]⁺. ¹H NMR (400 MHz, CDCl₃) δ 6.46-5.97 (m, 4H), 5.73-5.03 (m, 4H), 4.70-4.54 (m, 5H), 4.50-4.12 (m, 2H), 3.93-3.73 (m, 3H), 3.72-3.50 (m, 8H), 3.49-3.03 (m, 13H), 2.98-2.51 (m, 4H), 2.38-1.87 (m, 7H), 1.83-1.55 (m, 17H), 1.54-1.15 (m, 10H), 1.14-0.81 (m, 17H), 0.80-0.68 (m, 1H).

Step 2: (1R,9S,12S,15R,16E,18R,19R,21R,23S,24E,26E,28E,30S,32S,35R)-1-hydroxy-12-[(1R)-2-[(1S,3R,4R)-4-(2-hydroxyethoxy)-3-methoxy-cyclohexyl]-1-methyl-ethyl]-18,19-dimethoxy-15,17,21,23,29,35-hexamethyl-30-[2-[2-(oxetan-3-yloxy)ethoxy]ethoxy]-11,36-dioxa-4-azatricyclo[30.3.1.0^{4,9}]hexatriaconta-16,24,26,28-tetraene-2,3,10,14,20-pentone (I-62). 129 mg of I-80 was separated via chiral preparatory HPLC and then purified via silica gel chromatography to provide I-62 (30.6 mg, 24% yield) as a white solid.

Chiral analysis method:

Column: CHIRALPAK IC (IC00CD-TB016)

Column size: 0.46 cm I.D.×15 cm L

Injection: 10 μl

Mobile phase: Hexane/EtOH=50/50 (V/V)

Flow rate: 1.0 mL/min

Wavelength: UV 254 nm

Temperature: 35° C.

HPLC equipment Shimadzu-LC-20AD

I-62: ESI-MS (EI⁺, m/z): 1124.3 [M+Na]⁺. ¹H NMR (400 MHz, CDCl₃) δ 6.51-5.80 (m, 4H), 5.73-5.03 (m, 4H), 4.86-4.53 (m, 5H), 3.99-3.03 (m, 31H), 2.99-2.50 (m, 4H), 2.40-1.83 (m, 10H), 1.82-1.44 (m, 9H), 1.43-1.18 (m, 9H), 1.18-0.67 (m, 19H).

### Example 45: Synthesis of (1R,9S,12S,15R,16E,18R,19R,21R,23S,24E,26E,28E,32S,35R)-30-[[(2S)-1,4-dioxan-2-yl]methoxy]-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-11,36-dioxa-4-azatricyclo[30.3.1.0^{4,9}]hexatriaconta-16,24,26,28-tetraene-2,3,10,14,20-pentone (I-76), (1R,9S,12S,15R,16E,18R,19R,21R,23S,24E,26E,28E,30S,32S,35R)-30-[[(2S)-1,4-dioxan-2-yl]methoxy]-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-11,36-dioxa-4-azatricyclo[30.3.1.0^{4,9}]hexatriaconta-16,24,26,28-tetraene-2,3,10,14,20-pentone (I-66) and (1R,9S,12S,15R,16E,18R,19R,21R,23S,24E,26E,28E,30R,32S,35R)-30-[[(2S)-1,4-dioxan-2-yl]methoxy]-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-11,36-dioxa-4-azatricyclo[30.3.1.0^{4,9}]hexatriaconta-16,24,26,28-tetraene-2,3,10,14,20-pentone (I-65)

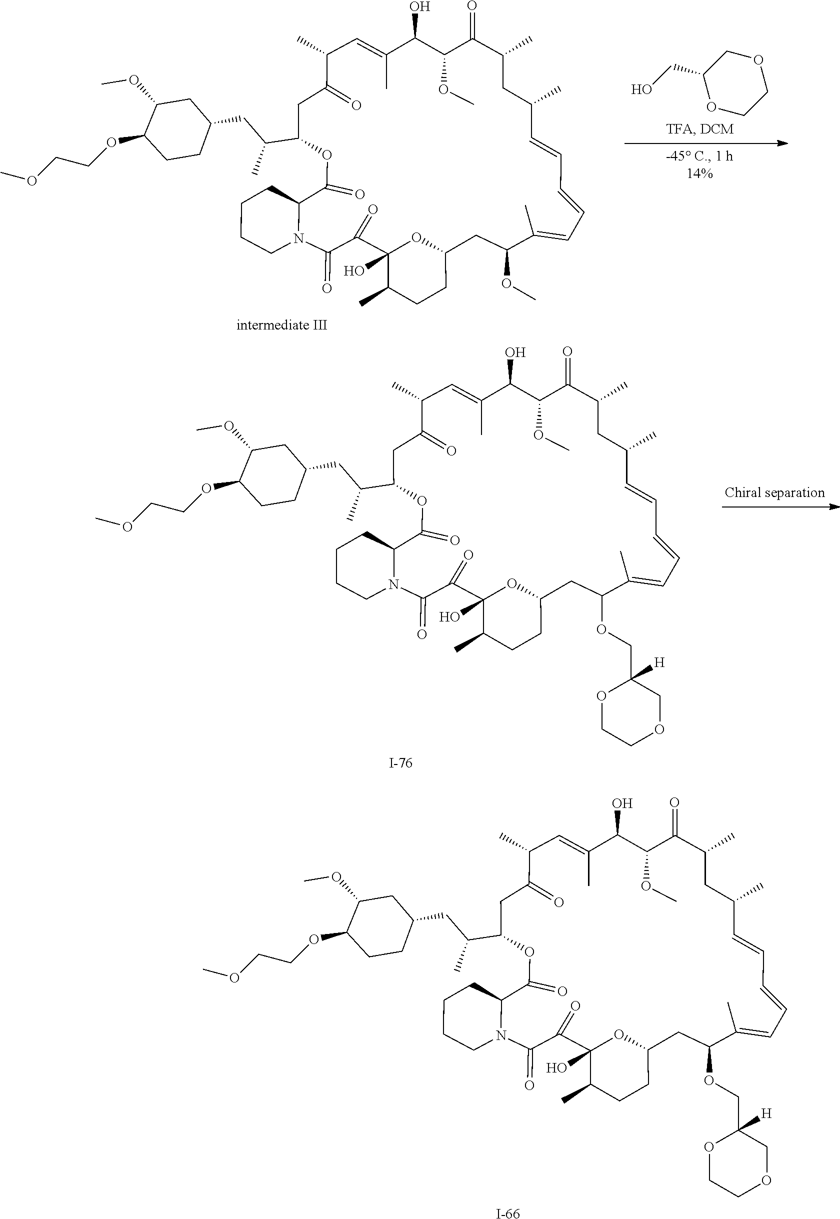

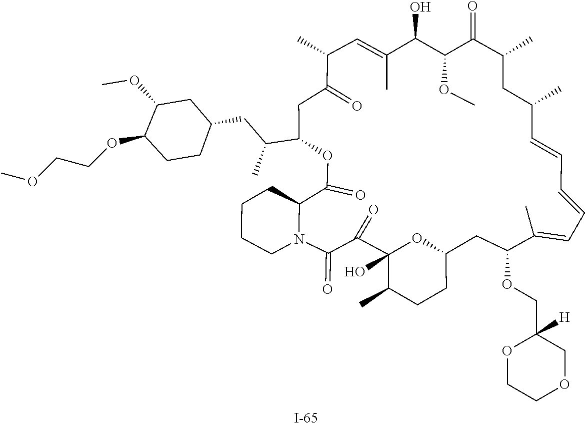

Step 1: (1R,9S,12S,15R,16E,18R,19R,21R,23S,24E,26E,28E,32S,35R)-30-[[(2S)-1,4-dioxan-2-yl]methoxy]-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-11,36-dioxa-4-azatricyclo[30.3.1.0^{4,9}]hexatriaconta-16,24,26,28-tetraene-2,3,10,14,20-pentone (I-76). To a solution of Intermediate III (0.5 g, 0.51 mmol) in DCM (40 mL) was added 2,2,2-trifluoroacetic acid (1.17 g, 10.29 mmol) dropwise at −55° C. under N₂. The reaction was stirred for 10 min then [(2R)-1,4-dioxan-2-yl]methanol (1.03 g, 8.74 mmol in DCM) was added at the same temperature. The reaction was warmed over 2 h to −10° C. then poured into saturated aqueous NaHCO₃ (40 mL) at 0° C. and extracted with DCM (50 mL). The organic layer was washed with water (50 mL) and brine (50 mL), dried over anhydrous sodium sulfate, filtered and concentrated. The residue was purified via silica gel chromatography (EA 100%) then by reverse phase chromatography eluting with 65% CH₃CN in water to provide I-76 (0.08 g, 15% yield) as a white solid. ESI-MS (EI⁺, m/z): 1080.7 [M+Na]⁺. ¹H NMR (400 MHz, CDCl₃) δ 6.39-5.92 (m, 4H), 5.56-4.81 (m, 5H), 4.28-3.98 (m, 3H), 3.90-3.68 (m, 9H), 3.65-3.28 (m, 16H), 3.26-2.97 (m, 5H), 2.88-2.46 (m, 4H), 2.35-1.91 (m, 6H), 1.89-1.60 (m, 18H), 1.55-1.16 (m, 10H), 1.14-0.83 (m, 18H), 1.76-0.65 (m, 1H).

Step 2: (1R,9S,12S,15R,16E,18R,19R,21R,23S,24E,26E,28E,30S,32S,35R)-30-[[(2S)-1,4-dioxan-2-yl]methoxy]-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-11,36-dioxa-4-azatricyclo[30.3.1.0^{4,9}]hexatriaconta-16,24,26,28-tetraene-2,3,10,14,20-pentone (I-66) and (1R,9S,12S,15R,16E,18R,19R,21R,23S,24E,26E,28E,30R,32S,35R)-30-[[(2S)-1,4-dioxan-2-yl]methoxy]-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-11,36-dioxa-4-azatricyclo[30.3.1.0^{4,9}]hexatriaconta-16,24,26,28-tetraene-2,3,10,14,20-pentone (I-65). 155 mg of I-76 was separated via chiral preparatory HPLC and then purified via silica gel chromatography to obtain I-66 (33.2 mg, 21.42% yield) as a white solid and I-65 (13.8 mg, 9% yield) as a white solid.

Chiral analysis method:

Column: CHIRALPAK IC (IC00CD-TB016)

Column size: 0.46 cm I.D.×15 cm L

Injection: 10 μl

Mobile phase: Hexane/EtOH=50/50 (V/V)

Flow rate: 1.0 mL/min

Wavelength: UV 254 nm

Temperature: 35° C.

HPLC equipment: Shimadzu-LC-20AD

I-66: ESI-MS (EI⁺, m/z): 1080.4 [M+Na]⁺. ¹H NMR (400 MHz, CDCl₃) δ 6.43-5.82 (m, 4H), 5.60-5.07 (m, 4H), 4.82 (s, 1H), 4.17 (d, J=5.7 Hz, 1H), 3.88-3.00 (m, 31H), 2.88-2.51 (m, 3H), 2.40-1.68 (m, 13H), 1.55-1.30 (m, 8H), 1.29-1.15 (m, 7H), 1.14-0.62 (m, 19H).
I-65: ESI-MS (EI⁺, m/z): 1080.4 [M+Na]⁺. ¹H NMR (400 MHz, CDCl₃) δ 6.45-5.87 (m, 4H), 5.71-5.10 (m, 4H), 4.10 (dd, J=85.6, 30.3 Hz, 3H), 3.86-2.83 (m, 30H), 2.82-1.71 (m, 17H), 1.54-1.14 (m, 14H), 1.12-0.59 (m, 19H).

### Example 46: Synthesis of (1R,9S,12S,15R,16E,18R,19R,21R,23S,24E,26E,28E,30S,32S,35R)-12-[(1R)-2-[(1S,3R,4R)-4-dimethylphosphoryloxy-3-methoxy-cyclohexyl]-1-methyl-ethyl]-1,18-dihydroxy-19-methoxy-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone (I-78) and (1R,9S,12S,15R,16E,18R,19R,21R,23S,24E,26E,28E,30R,32S,35R)-12-[(1R)-2-[(1S,3R,4R)-4-dimethylphosphoryloxy-3-methoxy-cyclohexyl]-1-methyl-ethyl]-1,18-dihydroxy-19-methoxy-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone (I-77)

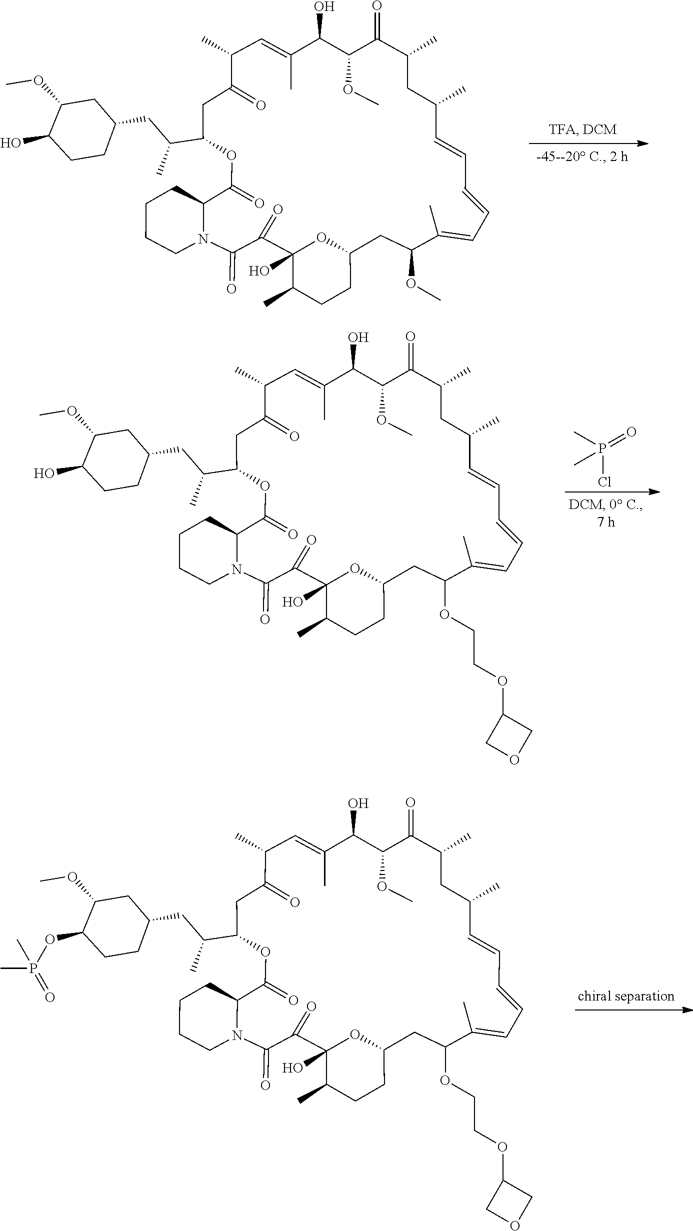

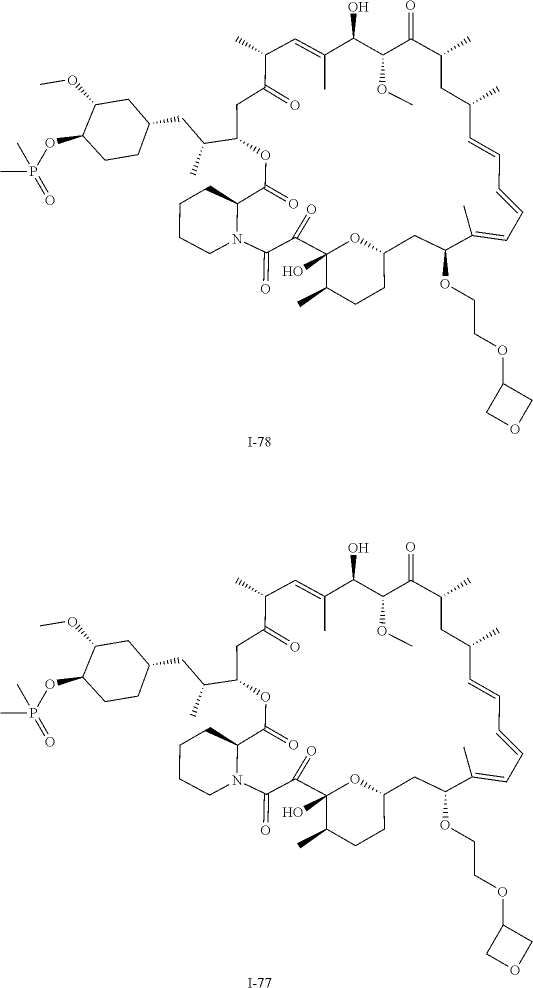

Step 1: (23E,25E,27E,28E,32R,33S,34R,35R,37S,39S,41R,42S,44R,45R,54R)-44,54-dihydroxy-42-[(1R)-2-[(1S,3R,4R)-4-hydroxy-3-methoxy-cyclohexyl]-1-methyl-ethyl]-45-methoxy-32,33,34,35,46,47-hexamethyl-41-[2-(oxetan-3-yloxy)ethoxy]-65,66-dioxa-55-azatricyclohexatriaconta-23,25,27(46),28(47)-tetraene-48,49,50,51,52-pentone. To a solution of rapamycin (0.5 g, 0.547 mmol) in DCM (10 mL) under nitrogen was added TFA (1.87 g, 16.41 mmol, 1.26 mL) at −40° C. 2-(oxetan-3-yloxy) ethanol (1.29 g, 10.94 mmol) was added and the mixture was stirred at −20° C. for 2h. The reaction mixture was quenched by adding saturated aqueous NaHCO₃(20 mL) and extracted with DCM (30 mL) at 0° C. The organic layer was washed with water (20 mL) and brine (20 mL), dried over anhydrous sodium sulfate, filtered and concentrated. The residue was purified by reverse phase column chromatography eluting with 80% CH₃CN in water to provide (23E,25E,27E,28E,32R,33S,34R,35R,37S,39S,41R,42S,44R,45R,54R)-44,54-dihydroxy-42-[(1R)-2-[(1S,3R,4R)-4-hydroxy-3-methoxy-cyclohexyl]-1-methyl-ethyl]-45-methoxy-32,33,34,35,46,47-hexamethyl-41-[2-(oxetan-3-yloxy)ethoxy]-65,66-dioxa-55-azatricyclohexatriaconta-23,25,27(46),28(47)-tetraene-48,49,50,51,52-pentone (120 mg, 22% yield) as a white solid. ESI-MS (EI⁺, m/z): 1022.7 [M+Na]⁺. ¹H NMR (400 MHz, CDCl₃) δ 6.42-5.88 (m, 4H), 5.58-5.08 (m, 4H), 4.83-4.54 (m, 5H), 4.35-3.93 (m, 2H), 3.91-3.68 (m, 3H), 3.53-3.21 (m, 13H), 2.99-2.41 (m, 5H), 2.38-1.87 (m, 7H), 1.85-1.58 (m, 13H), 1.55-1.17 (m, 11H), 1.16-0.82 (m, 17H), 0.80-0.63 (m, 1H).

Step 2: (1R,9S,12S,15R,16E,18R,19R,21R,23S,24E,26E,28E,32S,35R)-12-[(1R)-2-[(1S,3R,4R)-4-dimethylphosphoryloxy-3-methoxy-cyclohexyl]-1-methyl-ethyl]-1,18-dihydroxy-19-methoxy-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone. To a solution of (1R,9S,12S,15R,16E,18R,19R,21R,23S,24E,26E,28E,32S,35R)-1,18-dihydroxy-12-[(1R)-2-[(1S,3R,4R)-4-hydroxy-3-methoxy-cyclohexyl]-1-methyl-ethyl]-19-methoxy-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone (0.29 g, 0.29 mmol) in DCM (7 mL) was added 2,6-di-tert-butyl-4-methylpyridine (0.45 g, 2.17 mmol) at 0° C. Dimethylphosphinic chloride (0.163 g, 1.45 mmol) in DCM (2 mL) was added and the reaction stirred at 0° C. for 7 h then diluted with DCM, washed with saturated NaHCO₃ (30 mL), 0.5N HCl aqueous solution, water (30 mL) and brine (50 mL). The organic layer was dried over Na₂SO₄, filtered, concentrated in vacuo and the residue purified via silica gel chromatography (MeOH in PE:EA:DCM (3:3:10) from 0 to 15%) and by reverse phase chromatography (60% CH₃CN in water) to provide (1R,9S,12S,15R,16E,18R,19R,21R,23S,24E,26E,28E,32S,35R)-12-[(1R)-2-[(1S,3R,4R)-4-dimethylphosphoryloxy-3-methoxy-cyclohexyl]-1-methyl-ethyl]-1,18-dihydroxy-19-methoxy-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone (130 mg, 42% yield) as a white solid. ESI-MS (EI⁺, m/z): 1098.7 [M+Na]⁺.

Step 3: (1R,9S,12S,15R,16E,18R,19R,21R,23S,24E,26E,28E,30S,32S,35R)-12-[(1R)-2-[(1S,3R,4R)-4-dimethylphosphoryloxy-3-methoxy-cyclohexyl]-1-methyl-ethyl]-1,18-dihydroxy-19-methoxy-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone (I-78) and (1R,9S,12S,15R,16E,18R,19R,21R,23S,24E,26E,28E,30R,32S,35R)-12-[(1R)-2-[(1S,3R,4R)-4-dimethylphosphoryloxy-3-methoxy-cyclohexyl]-1-methyl-ethyl]-1,18-dihydroxy-19-methoxy-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone (I-77). 150 mg of (1R,9S,12S,15R,16E,18R,19R,21R,23S,24E,26E,28E,32S,35R)-12-[(1R)-2-[(1S,3R,4R)-4-dimethylphosphoryloxy-3-methoxy-cyclohexyl]-1-methyl-ethyl]-1,18-dihydroxy-19-methoxy-15,17,21,23,29,35-hexamethyl-30-[2-(oxetan-3-yloxy)ethoxy]-11,36-dioxa-4-azatricyclo[30.3.1.0^{4,9}]hexatriaconta-16,24,26,28-tetraene-2,3,10,14,20-pentone was separated via chiral preparatory HPLC and then purified via silica gel chromatography to provide I-78 (28.5 mg, 19% yield) as a white solid and I-77 (12.3 mg, 8% yield) as a white solid.

Chiral analysis method:

Column: CHIRALPAK IC (IC00CE-QE014)

Column size: 0.46 cm I.D.×25 cm L

Injection: 10 μL

Mobile phase: Hexane/EtOH=40/60 (V/V)

Flow rate: 1.0 mL/min

Wavelength: UV 254 nm

Temperature: 35° C.

HPLC equipment: Shimadzu LC-20AD

I-78: ESI-MS (EI⁺, m/z): 1098.7 [M+Na]⁺. ¹H NMR (400 MHz, CDCl₃) δ 6.37-5.78 (m, 4H), 5.52-5.02 (m, 4H), 4.79-4.44 (m, 5H), 4.24-3.94 (m, 2H), 3.89-3.57 (m, 3H), 3.55-2.88 (m, 15H), 2.80-2.42 (m, 3H), 2.36-1.78 (m, 8H), 1.75-1.35 (m, 16H), 1.32-1.10 (m, 11H), 1.08-0.57 (m, 19H).
I-77: ESI-MS (EI⁺, m/z): 1098.7 [M+Na]⁺. ¹H NMR (400 MHz, CDCl₃) δ 6.39-5.93 (m, 4H), 5.65-5.01 (m, 4H), 4.80-4.46 (m, 5H), 4.26-3.91 (m, 4H), 3.51-3.10 (m, 13H), 3.04-1.91 (m, 11H), 1.86-1.52 (m, 20H), 1.49-1.11 (m, 10H), 1.08-0.57 (m, 19H).

### Example 47: Synthesis of (1R,9S,12S,15R,16E,18R,19R,21R,23S,24E,26E,28E,32S,35R)-30-[[(2R)-1,4-dioxan-2-yl]methoxy]-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-11,36-dioxa-4-azatricyclo[30.3.1.0^{4,9}]hexatriaconta-16,24,26,28-tetraene-2,3,10,14,20-pentone (I-79), (1R,9S,12S,15R,16E,18R,19R,21R,23S,24E,26E,28E,30S,32S,35R)-30-[[(2R)-1,4-dioxan-2-yl]methoxy]-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-11,36-dioxa-4-azatricyclo[30.3.1.0^{4,9}]hexatriaconta-16,24,26,28-tetraene-2,3,10,14,20-pentone (I-64) and (1R,9S,12S,15R,16E,18R,19R,21R,23S,24E,26E,28E,30R,32S,35R)-30-[[(2R)-1,4-dioxan-2-yl]methoxy]-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-11,36-dioxa-4-azatricyclo[30.3.1.0^{4,9}]hexatriaconta-16,24,26,28-tetraene-2,3,10,14,20-pentone (I-63)

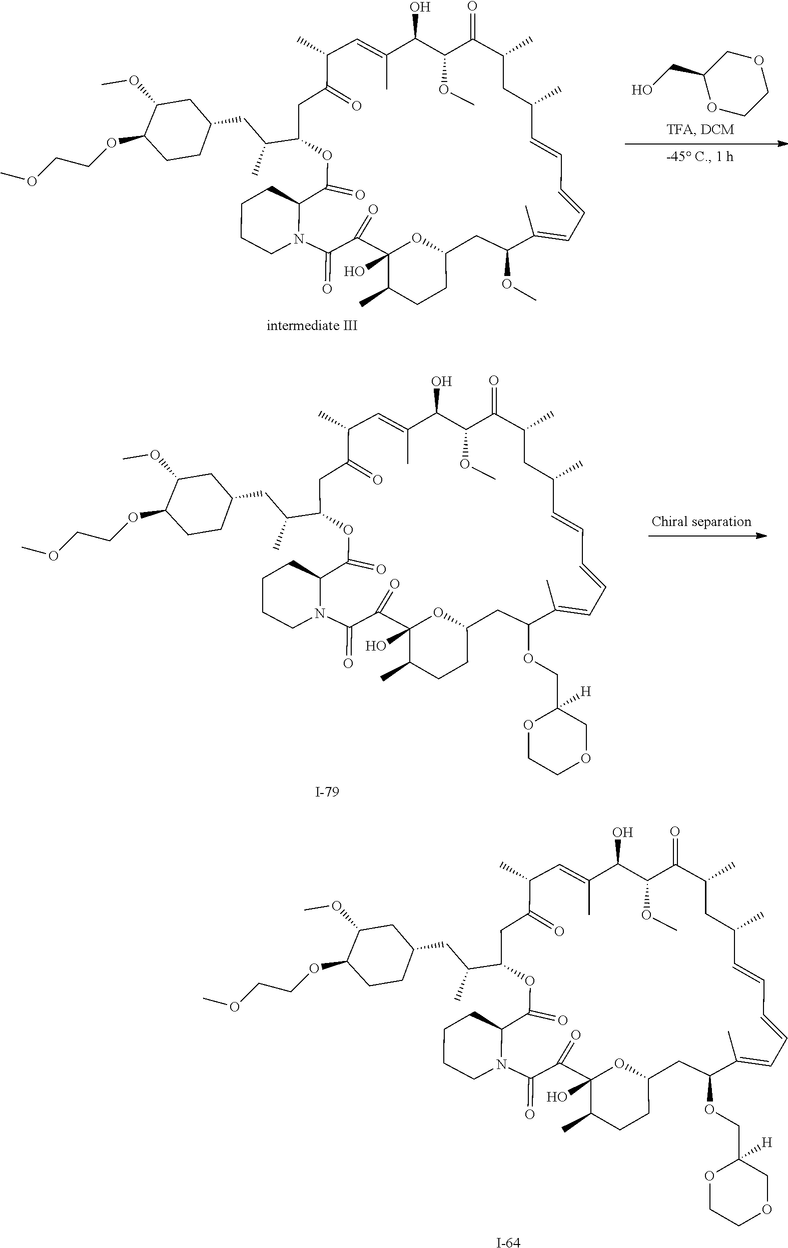

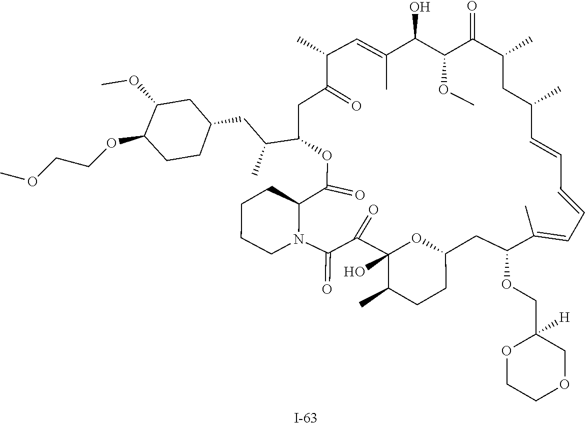

Step 1: (1R,9S,12S,15R,16E,18R,19R,21R,23S,24E,26E,28E,32S,35R)-30-[[(2R)-1,4-dioxan-2-yl]methoxy]-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-11,36-dioxa-4-azatricyclo[30.3.1.0^{4,9}]hexatriaconta-16,24,26,28-tetraene-2,3,10,14,20-pentone (I-79). To a solution of Intermediate III (0.5 g, 0.51 mmol) in DCM (35 mL) was added 2,2,2-trifluoroacetic acid (1.17 g, 10.29 mmol, −0.79 mL) dropwise at −55° C. under N₂. The reaction was stirred for 10 min at −45° C., [(2S)-1,4-dioxan-2-yl]methanol (0.97 g, 8.23 mmol in DCM) was added then the mixture was warmed to −10° C. over 1h. The reaction was poured into saturated aqueous NaHCO₃ (40 mL) at 0° C. and extracted with DCM (40 mL). The organic layer was washed with water (40 mL) and brine (40 mL), dried over anhydrous sodium sulfate, filtered and concentrated. The residue was purified via silica gel chromatography (100% EA) then re-purified by reverse phase chromatography eluting with 60% CH₃CN in water to provide I-79 (0.1 g, 18% yield) as a white solid. ESI-MS (EI⁺, m/z): 1080.6 [M+Na]⁺. ¹H NMR (400 MHz, CDCl₃) δ 6.39-5.92 (m, 4H), 5.57-4.77 (m, 5H), 4.31-3.98 (m, 3H), 3.85-3.67 (m, 8H), 3.65-3.24 (m, 17H), 3.22-2.97 (m, 3H), 2.75-2.26 (m, 5H), 2.17-1.90 (m, 5H), 1.86-1.58 (m, 17H), 1.51-1.16 (m, 10H), 1.15-0.81 (m, 18H), 0.76-0.65 (m, 1H).

Step 2: (1R,9S,12S,15R,16E,18R,19R,21R,23S,24E,26E,28E,30S,32S,35R)-30-[[(2R)-1,4-dioxan-2-yl]methoxy]-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-11,36-dioxa-4-azatricyclo[30.3.1.0^{4,9}]hexatriaconta-16,24,26,28-tetraene-2,3,10,14,20-pentone (I-64) and (1R,9S,12S,15R,16E,18R,19R,21R,23S,24E,26E,28E,30R,32S,35R)-30-[[(2R)-1,4-dioxan-2-yl]methoxy]-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-11,36-dioxa-4-azatricyclo[30.3.1.0^{4,9}]hexatriaconta-16,24,26,28-tetraene-2,3,10,14,20-pentone (I-63). 146 mg of I-79 was separated via chiral preparatory HPLC and then purified via silica gel chromatography to provide I-64 (31.2 mg, 21% yield) as a white solid and I-63 (15.4 mg, 11% yield) as a white solid.

Chiral analysis method:

Column: CHIRALPAK IC (IC00CE-BN011)

Column size: 0.46 cm I.D.×25 cm L

Injection: 10 μL

Mobile phase: Hexane/EtOH=50/50 (V/V)

Flow rate: 1.0 mL/min

Wavelength: UV 254 nm

Temperature: 35° C.

HPLC equipment: Shimadzu-LC-20AD

I-64: ESI-MS (EI⁺, m/z): 1080.3 [M+Na]⁺. ¹H NMR (400 MHz, CDCl₃) δ 6.43-5.81 (m, 4H), 5.58-5.08 (m, 4H), 4.77 (s, 1H), 4.17 (d, J=5.6 Hz, 1H), 3.89-3.26 (m, 28H), 3.22-2.99 (m, 4H), 2.89-2.46 (m, 3H), 2.38-1.67 (m, 13H), 1.55-1.16 (m, 13H), 1.13-0.59 (m, 20H).
I-63: ESI-MS (EI⁺, m/z): 1080.3 [M+Na]⁺. ¹H NMR (400 MHz, CDCl₃) δ 6.47-5.93 (m, 4H), 5.70-5.14 (m, 4H), 4.34-3.94 (m, 3H), 3.86-2.93 (m, 30H), 2.87-1.87 (m, 9H), 1.72 (t, J=14.6 Hz, 8H), 1.51-1.16 (m, 12H), 1.13-0.59 (m, 21H).

### Example 48: Synthesis of (1R,2R,4S)-4-((2R)-2-((3S,6R,7E,9R,10R,12R,14S,15E,17E,19E,23S,26R,27R,34aS)-27-hydroxy-9,10-dimethoxy-6,8,12,14,20,26-hexamethyl-21-(2-(oxetan-3-yloxy)ethoxy)-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)-2-methoxycyclohexyl dimethylphosphinate (I-75)

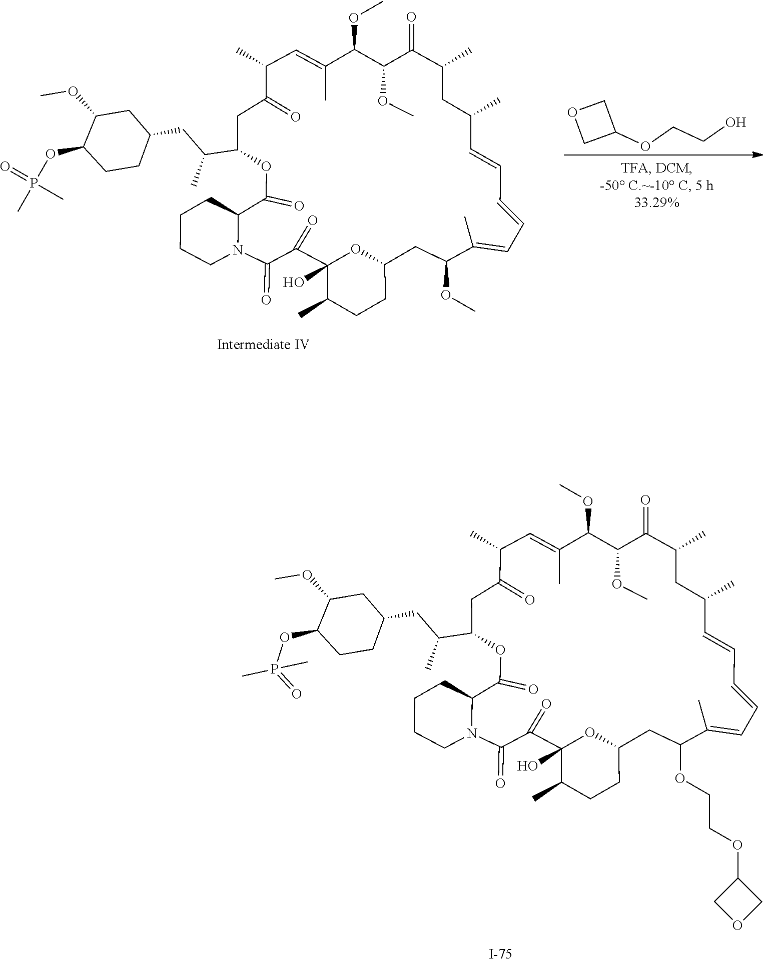

To a solution of Intermediate IV (0.5 g, 0.498 mmol) in DCM (15 mL) was added TFA (2.27 g, 19.92 mmol, 1.53 mL) at −50° C. After 10 minutes 2-(oxetan-3-yloxy) ethanol (1.76 g, 14.94 mmol) in DCM (0.5 mL) was added and the mixture stirred at −10° C. for 5 h. The reaction was diluted with DCM and aqueous NaHCO₃ solution, washed with water and brine, dried over Na₂SO₄, filtered and concentrated. The residue was purified via reverse-phase chromatography to provide I-75 (180.7 mg, 33% yield) as a white solid. ESI-MS (EI+, m/z): 1112.5 [M+Na]⁺. ¹H NMR (500 MHz, CDCl₃) δ 6.47-5.79 (m, 4H), 5.38 (dddd, J=91.9, 76.3, 49.1, 19.1 Hz, 4H), 4.80-4.03 (m, 7H), 3.94-2.94 (m, 22H), 2.93-1.83 (m, 11H), 1.67-1.30 (m, 22H), 1.30-0.82 (m, 21H), 0.77 (dd, J=24.4, 12.2 Hz, 1H).

### Example 49: Synthesis of (1R,2R,4S)-4-((2R)-2-((3S,6R,7E,9R,10R,12R,14S,15E,17E,19E,23S,26R,27R,34aS)-27-hydroxy-9,10-dimethoxy-6,8,12,14,20,26-hexamethyl-21-(3-morpholinopropoxy)-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c]1]oxa[4]azacyclohentriacontin-3-yl)propyl)-2-methoxycyclohexyl dimethylphosphinate (I-74)

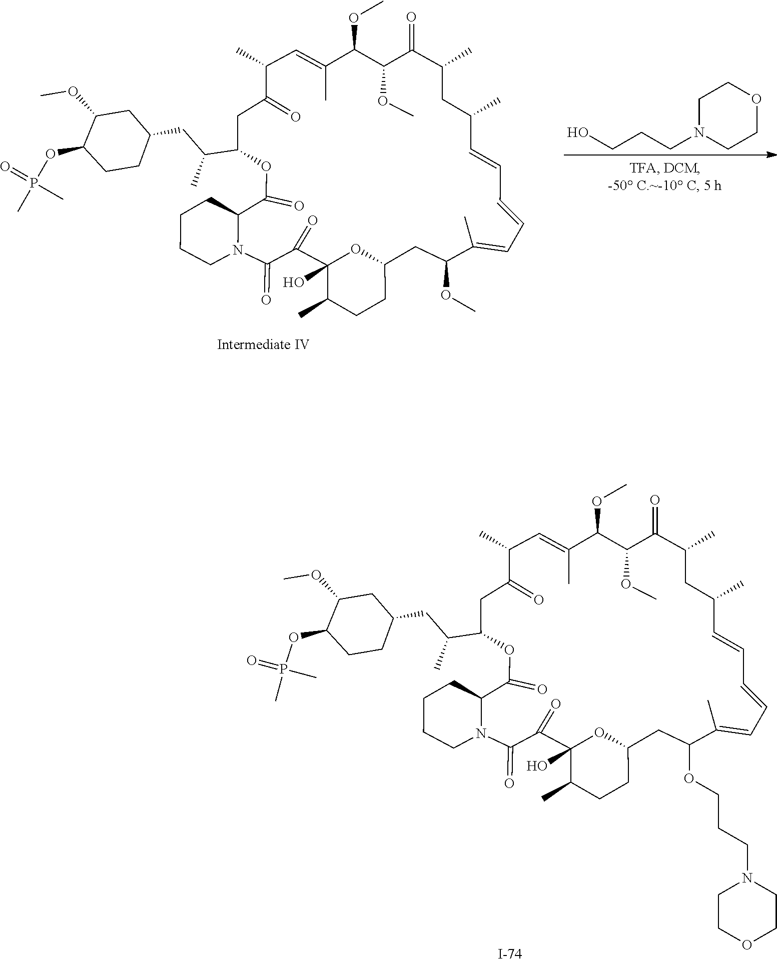

To a solution of Intermediate IV (0.35 g, 0.349 mmol) in DCM (15 mL) was added TFA (1.59 g, 13.94 mmol) at −50° C. The reaction was stirred 10 minutes then 3-morpholinopropan-1-ol (1.52 g, 10.46 mmol) dissolved in DCM (0.5 mL) was added and the mixture stirred at −10° C. for 5 h. The mixture was diluted with DCM and aqueous NaHCO₃ solution, washed with water and brine, dried over Na₂SO₄, filtered and concentrated. The residue was purified via reverse-phase chromatography to provide I-74 (138.8 mg, 36% yield) as a white solid. ESI-MS (EI⁺, m/z): 1118.7 [M+H]⁺. ¹H NMR (400 MHz, CDCl₃) δ 6.19 (dd, J=78.7, 69.0 Hz, 4H), 5.32 (d, J=60.0 Hz, 4H), 4.11 (s, 2H), 3.93-3.54 (m, 9H), 3.47-2.93 (m, 18H), 2.90-1.93 (m, 17H), 1.32 (dd, J=60.9, 36.3 Hz, 17H), 1.19-0.62 (m, 26H).

### Example 49: (1R,9S,12S,15R,16E,18R,19R,21R,23S,24E,26E,28E,32S,35R)-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-30-(pyrazin-2-ylmethoxy)-11,36-dioxa-4-azatricyclo[30.3.1.0^{4,9}]hexatriaconta-16,24,26,28-tetraene-2,3,10,14,20-pentone (I-68), (1R,9S,12S,15R,16E,18R,19R,21R,23S,24E,26E,28E,30S,32S,35R)-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-30-(pyrazin-2-ylmethoxy)-11,36-dioxa-4-azatricyclo[30.3.1.0^{4,9}]hexatriaconta-16,24,26,28-tetraene-2,3,10,14,20-pentone (I-57) and (1R,9S,12S,15R,16E,18R,19R,21R,23S,24E,26E,28E,30R,32S,35R)-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-30-(pyrazin-2-ylmethoxy)-11,36-dioxa-4-azatricyclo[30.3.1.0^{4,9}]hexatriaconta-16,24,26,28-tetraene-2,3,10,14,20-pentone (I-56)

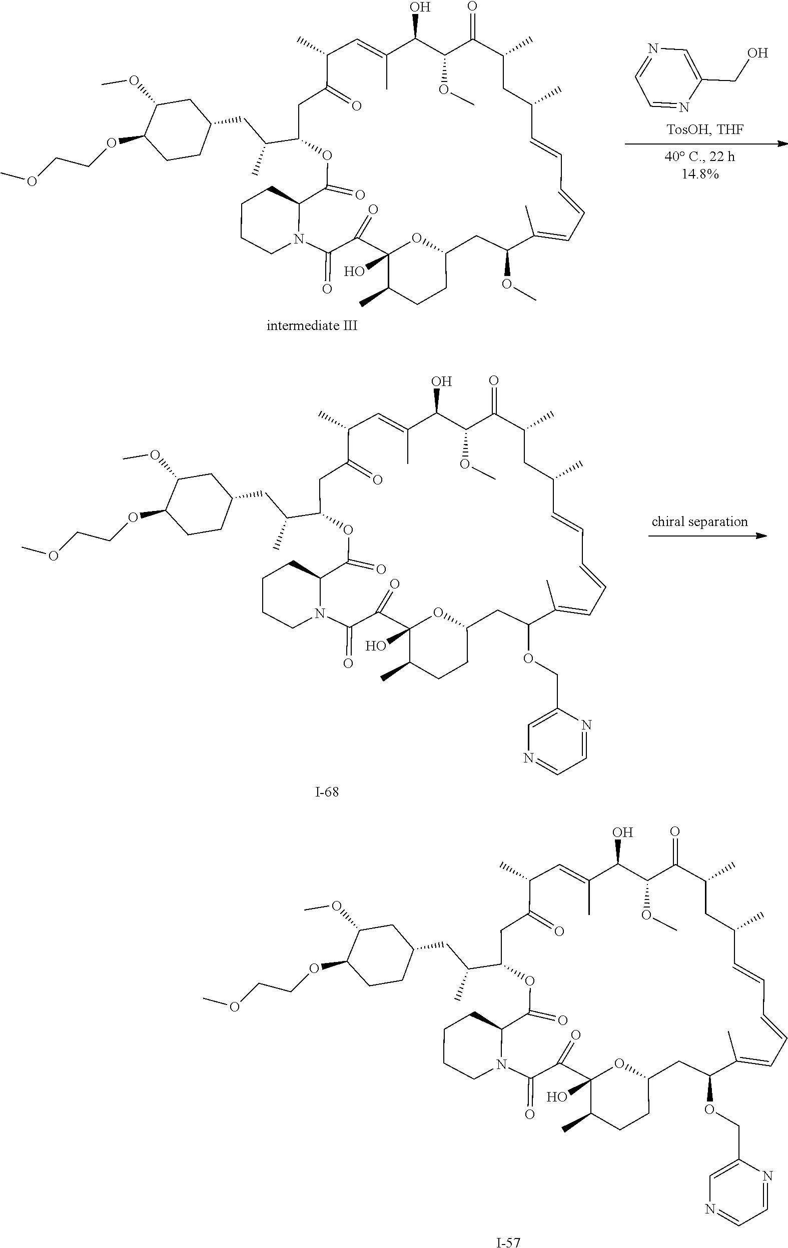

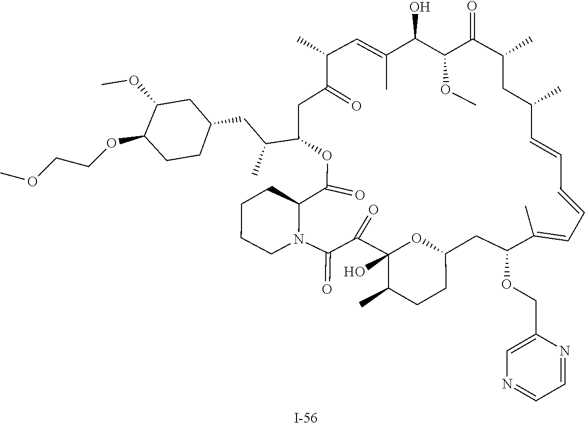

Step 1: (1R,9S,12S,15R,16E,18R,19R,21R,23S,24E,26E,28E,32S,35R)-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-30-(pyrazin-2-ylmethoxy)-11,36-dioxa-4-azatricyclo[30.3.1.0^{4,9}]hexatriaconta-16,24,26,28-tetraene-2,3,10,14,20-pentone (I-68). To a solution of (23E,25E,27E,28E,32R,33S,34R,35R,37S,39S,41S,42S,43R,44R,53R)-43,53-dihydroxy-41,44-dimethoxy-42-[(1R)-2-[(1S,3R,4R)-3-methoxy-4-(2-methoxyethoxy)cyclohexyl]-1-methyl-ethyl]-32,33,34,35,45,46-hexamethyl-62,63-dioxa-54-azatricyclohexatriaconta-23,25,27(45),28(46)-tetraene-47,48,49,50,51-pentone (0.5 g, 0.51 mmol) and pyrazin-2-ylmethanol (0.96 g, 8.74 mmol) in THF (15 mL) was added 4-methylbenzenesulfonic acid hydrate (0.49 g, 2.57 mmol, 0.395 mL) at 0° C. under N₂. The reaction was stirred for 22 h at 40° C. then poured into cold saturated aqueous NaHCO₃ (30 mL) and extracted with DCM (10 mL). The organic layer was washed with water (30 mL) and brine (30 mL), dried over anhydrous sodium sulfate, filtered and concentrated. The residue was purified via silica gel chromatography (100% EA) then by reverse phase chromatography eluting with 70% CH₃CN in water to provide I-68 (0.08 g, 15% yield) as a light-yellow solid. ESI-MS (EI⁺, m/z): 1072.5 [M+Na]⁺. ¹H NMR (400 MHz, CDCl₃) δ 8.73-8.70 (m, 1H), 8.56-8.48 (m, 2H), 6.42-5.98 (m, 4H), 5.60-4.82 (m, 4H), 4.62-4.15 (m, 4H), 4.07-3.86 (m, 2H), 3.75-3.48 (m, 6H), 3.47-3.20 (m, 12H), 3.16-2.95 (m, 4H), 2.98-2.10 (m, 6H), 2.05-1.54 (m, 23H), 1.56-1.16 (m, 10H), 1.15-0.82 (m, 19H), 0.79-0.64 (m, 1H).

Step 2: (1R,9S,12S,15R,16E,18R,19R,21R,23S,24E,26E,28E,30S,32S,35R)-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-30-(pyrazin-2-ylmethoxy)-11,36-dioxa-4-azatricyclo[30.3.1.0^{4,9}]hexatriaconta-16,24,26,28-tetraene-2,3,10,14,20-pentone (I-57) and (1R,9S,12S,15R,16E,18R,19R,21R,23S,24E,26E,28E,30R,32S,35R)-1,18-dihydroxy-19-methoxy-12-[(1R)-2-[(1S,3R,4R)-3-methoxy-4-(2-methoxyethoxy)cyclohexyl]-1-methyl-ethyl]-15,17,21,23,29,35-hexamethyl-30-(pyrazin-2-ylmethoxy)-11,36-dioxa-4-azatricyclo[30.3.1.0^{4,9}]hexatriaconta-16,24,26,28-tetraene-2,3,10,14,20-pentone (I-56). 200 mg of I-68 was separated via chiral preparatory HPLC and then purified via silica gel chromatography (11% MeOH in PE:DCM:EA 3:3:1) to provide I-57 (24.4 mg, 12% yield) as a white solid and I-56 (21.5 mg, 10% yield) as a white solid.

Chiral analysis method:

Column: CHIRALPAK IC (IC00CD-TB016)

Column size: 0.46 cm I.D.×15 cm L

Injection: 10 μl

Mobile phase: Hexane/EtOH=50/50 (V/V)

Flow rate: 1.0 mL/min

Wavelength: UV 254 nm

Temperature: 35° C.

HPLC equipment: Shimadzu LC-20AT

I-57: ESI-MS (EI⁺, m/z): 1072.6 [M+Na]⁺. ¹H NMR (500 MHz, CDCl₃) δ 8.71 (s, 1H), 8.49 (d, J=2.4 Hz, 2H), 6.41-5.90 (m, 4H), 5.59-5.08 (m, 4H), 4.91 (s, 1H), 4.58 (d, J=13.8 Hz, 1H), 4.44-4.32 (m, 1H), 4.18 (t, J=16.3 Hz, 1H), 3.94 (dd, J=21.3, 14.0 Hz, 2H), 3.71 (ddd, J=25.5, 13.1, 7.6 Hz, 3H), 3.60-3.26 (m, 15H), 3.22-2.95 (m, 3H), 2.86-2.54 (m, 3H), 2.37-2.16 (m, 2H), 2.01 (dd, J=31.2, 14.8 Hz, 5H), 1.70 (dd, J=31.5, 12.5 Hz, 9H), 1.51-1.16 (m, 11H), 1.14-0.79 (m, 18H), 0.71 (dd, J=23.8, 12.1 Hz, 1H).
I-56: ESI-MS (EI⁺, m/z): 1072.7 [M+Na]⁺. ¹H NMR (500 MHz, CDCl₃) δ 8.73 (s, 1H), 8.51 (d, J=2.5 Hz, 2H), 6.43-5.78 (m, 4H), 5.71-5.01 (m, 4H), 4.65-4.16 (m, 4H), 4.03-3.61 (m, 4H), 3.56-2.90 (m, 18H), 2.85-1.69 (m, 16H), 1.41 (ddd, J=79.6, 43.9, 14.6 Hz, 14H), 1.14-0.59 (m, 20H).

### Example 50: Synthesis of (3S,6R,7E,9R,10R,12R,14S,15E,17E,19E,23S,26R,27R,34aS)-9,27-dihydroxy-10-methoxy-3-((R)-1-((1S,3R,4R)-3-methoxy-4-(2-methoxyethoxy)cyclohexyl)propan-2-yl)-6,8,12,14,20,26-hexamethyl-21-(2-(pyrazin-2-yl)ethoxy)-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (I-60)

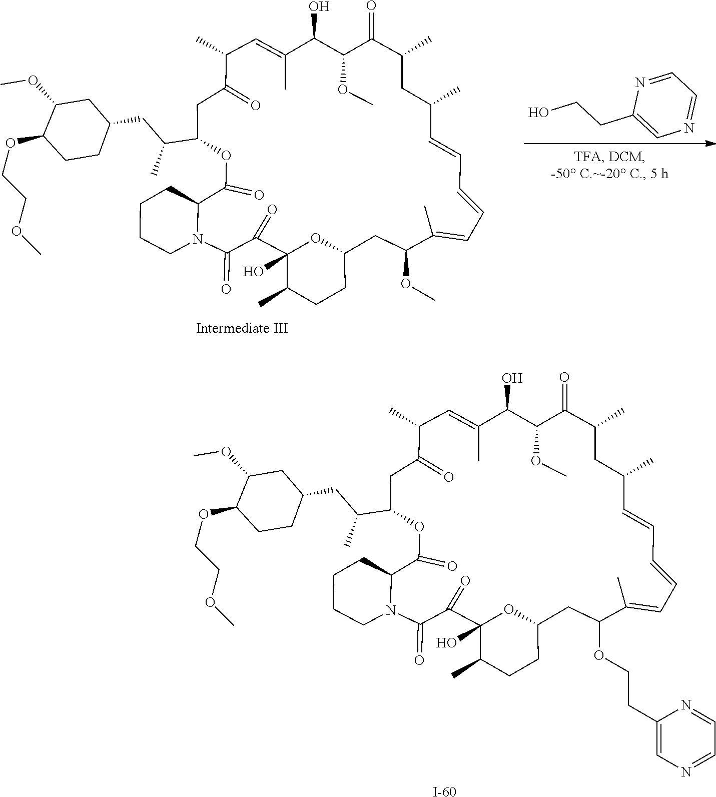

To a solution of Intermediate III (0.5 g, 0.51 mmol) in DCM (15 mL) was added TFA (2.35 g, 20.57 mmol) at −50° C. The reaction was stirred for 10 minutes then 2-pyrazin-2-ylethanol (1.92 g, 15.43 mmol) dissolved in DCM (0.5 mL) was added and the mixture stirred at −20° C. for 5 h. The mixture was diluted with DCM and aqueous NaHCO₃ solution then the organic layer was washed with water and brine, dried over Na₂SO₄, filtered and concentrated. The residue was purified via reverse-phase chromatography to provide I-60 (162.8 mg, 30% yield) as a white solid. ESI-MS (EI⁺, m/z): 1086.6 [M+Na]⁺. ¹H NMR (500 MHz, CDCl₃) δ 8.65-8.29 (m, 1H), 6.53-5.95 (m, 4H), 5.56-5.14 (m, 4H), 4.59-3.65 (m, 6H), 3.62-2.43 (m, 25H), 2.15 (dt, J=144.5, 40.3 Hz, 6H), 1.56-1.16 (m, 16H), 1.15-0.54 (m, 26H).

### Example 51: (1R,9S,12S,15R,16E,18R,19R,21R,23S,24E,26E,28E,32S,35R)-1,18-dihydroxy-12-[(1R)-2-[(1S,3R,4R)-4-(2-hydroxyethoxy)-3-methoxy-cyclohexyl]-1-methyl-ethyl]-19-methoxy-15,17,21,23,29,35-hexamethyl-30-(pyrazin-2-ylmethoxy)-11,36-dioxa-4-azatricyclo[30.3.1.0^{4,9}]hexatriaconta-16,24,26,28-tetraene-2,3,10,14,20-pentone (I-67), (1R,9S,12S,15R,16E,18R,19R,21R,23S,24E,26E,28E,30S,32S,35R)-1,18-dihydroxy-12-[(1R)-2-[(1 S,3R,4R)-4-(2-hydroxyethoxy)-3-methoxy-cyclohexyl]-1-methyl-ethyl]-19-methoxy-15,17,21,23,29,35-hexamethyl-30-(pyrazin-2-ylmethoxy)-11,36-dioxa-4-azatricyclo[30.3.1.0^{4,9}]hexatriaconta-16,24,26,28-tetraene-2,3,10,14,20-pentone (I-59) and (1R,9S,12S,15R,16E,18R,19R,21R,23S,24E,26E,28E,30R,32S,35R)-1,18-dihydroxy-12-[(1R)-2-[(1 S,3R,4R)-4-(2-hydroxyethoxy)-3-methoxy-cyclohexyl]-1-methyl-ethyl]-19-methoxy-15,17,21,23,29,35-hexamethyl-30-(pyrazin-2-ylmethoxy)-11,36-dioxa-4-azatricyclo[30.3.1.0^{4,9}]hexatriaconta-16,24,26,28-tetraene-2,3,10,14,20-pentone (I-58)

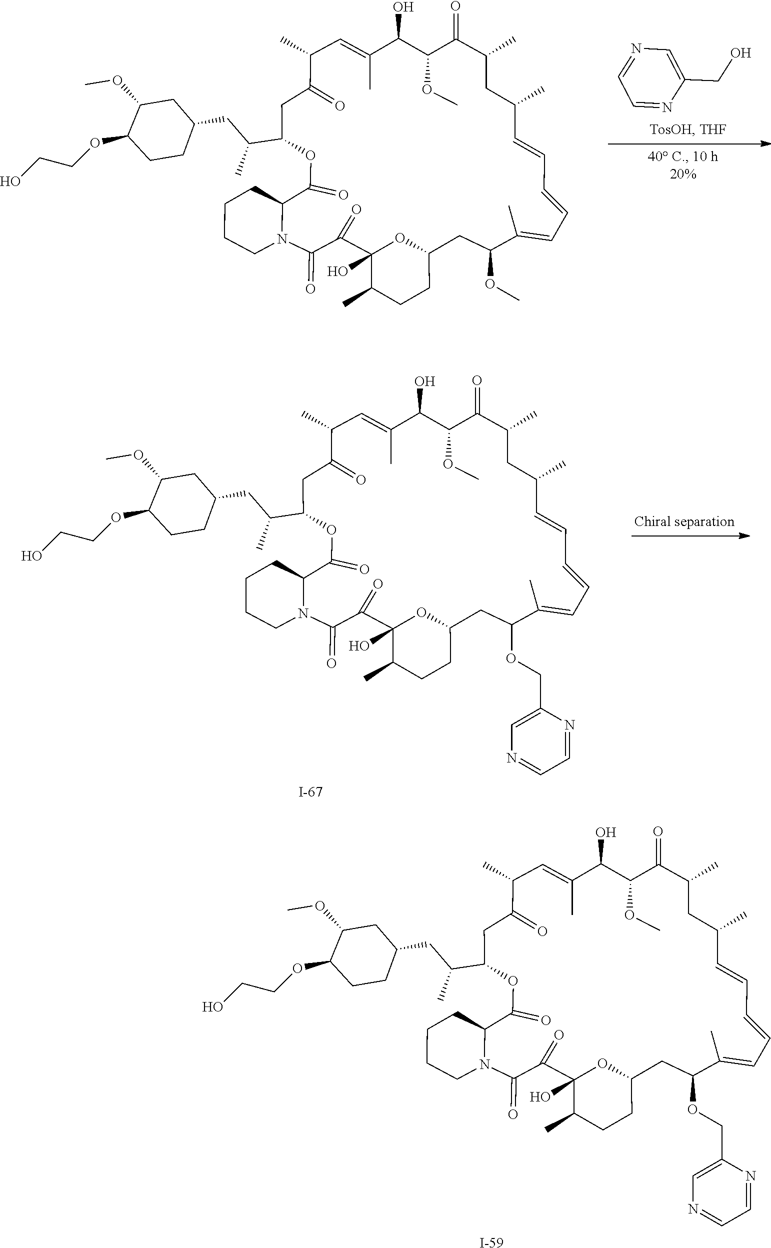

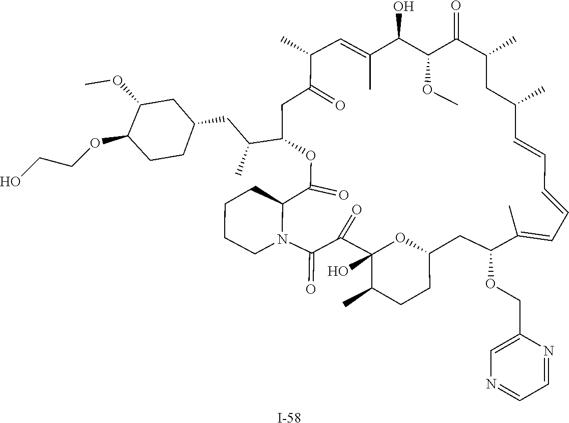

Step 1: (1R,9S,12S,15R,16E,18R,19R,21R,23S,24E,26E,28E,32S,35R)-1,18-dihydroxy-12-[(1R)-2-[(1S,3R,4R)-4-(2-hydroxyethoxy)-3-methoxy-cyclohexyl]-1-methyl-ethyl]-19-methoxy-15,17,21,23,29,35-hexamethyl-30-(pyrazin-2-ylmethoxy)-11,36-dioxa-4-azatricyclo[30.3.1.0^{4,9}]hexatriaconta-16,24,26,28-tetraene-2,3,10,14,20-pentone (I-67). To a solution of everolimus (0.977 g, 8.87 mmol) in THF (15 mL) was added 4-methylbenzenesulfonic acid hydrate (0.496 g, 2.61 mmol) at 0° C. under N₂. The reaction was stirred for 10 h at 40° C. then poured into cold saturated aqueous NaHCO₃ (30 mL) and extracted with EA (30 mL). The organic layer was washed with (30 mL) water and brine (30 mL), dried over anhydrous sodium sulfate, filtered and concentrated. The residue was purified via silica gel chromatography (DCM:MeOH=1:5) then by reverse phase chromatography eluting with 60% CH₃CN in water to give I-67 (0.11 g, 20% yield) as a light-yellow solid. ESI-MS (EI⁺, m/z): 1059.6 [M+Na]⁺. ¹H NMR (400 MHz, CDCl₃) δ 8.743-8.70 (m, 1H), 8.56-8.48 (m, 2H), 6.42-5.93 (m, 4H), 5.60-4.89 (m, 5H), 4.63-4.06 (m, 4H), 4.017-3.53 (m, 7H), 3.46-3.28 (m, 8H), 3.25-2.91 (m, 4H), 3.16-2.95 (m, 4H), 2.88-2.42 (m, 4H), 2.32-1.97 (m, 8H), 1.96-1.61 (m, 23H), 1.56-1.13 (m, 12H), 1.11-0.82 (m, 17H), 0.76-0.63 (m, 1H).

Step 2: (1R,9S,12S,15R,16E,18R,19R,21R,23S,24E,26E,28E,30S,32S,35R)-1,18-dihydroxy-12-[(1R)-2-[(1S,3R,4R)-4-(2-hydroxyethoxy)-3-methoxy-cyclohexyl]-1-methyl-ethyl]-19-methoxy-15,17,21,23,29,35-hexamethyl-30-(pyrazin-2-ylmethoxy)-11,36-dioxa-4-azatricyclo[30.3.1.0^{4,9}]hexatriaconta-16,24,26,28-tetraene-2,3,10,14,20-pentone (I-59) and (1R,9S,12S,15R,16E,18R,19R,21R,23S,24E,26E,28E,30R,32S,35R)-1,18-dihydroxy-12-[(1R)-2-[(1S,3R,4R)-4-(2-hydroxyethoxy)-3-methoxy-cyclohexyl]-1-methyl-ethyl]-19-methoxy-15,17,21,23,29,35-hexamethyl-30-(pyrazin-2-ylmethoxy)-11,36-dioxa-4-azatricyclo[30.3.1.0^{4,9}]hexatriaconta-16,24,26,28-tetraene-2,3,10,14,20-pentone (I-58). 220 mg of I-67 was separated via chiral preparatory HPLC and then purified via silica gel chromatography (11% MeOH in PE:DCM:EA 3:3:1) to provide I-59 (40.2 mg, 18% yield) as a white solid and I-58 (32.1 mg, 15% yield) as a white solid.

Chiral analysis method:

Column: CHIRALPAK IC (IC00CD-TB016)

Column size: 0.46 cm I.D.×15 cm L

Injection: 10 μL

Mobile phase: Hexane/EtOH=50/50 (V/V)

Flow rate: 1.0 mL/min

Wavelength: UV 254 nm

Temperature: 35° C.

HPLC equipment: Shimadzu LC-20AT

I-59: ESI-MS (EI⁺, m/z): 1059.6 [M+Na]⁺. ¹H NMR (500 MHz, CDCl₃) δ 8.71 (s, 1H), 8.49 (d, J=2.0 Hz, 2H), 6.45-5.91 (m, 4H), 5.60-5.09 (m, 4H), 4.90 (s, 1H), 4.58 (dd, J=13.6, 4.1 Hz, 1H), 4.46-4.33 (m, 1H), 4.19 (dd, J=20.7, 6.5 Hz, 1H), 4.02-3.51 (m, 8H), 3.48-3.02 (m, 12H), 2.88-2.54 (m, 3H), 2.36-1.88 (m, 7H), 1.85-1.63 (m, 11H), 1.52-1.17 (m, 10H), 1.14-0.79 (m, 18H), 0.75-0.64 (m, 1H).
I-58: ESI-MS (EI⁺, m/z): 1059.0 [M+Na]⁺. ¹H NMR (500 MHz, CDCl₃) δ 8.79 (s, 1H), 8.51 (d, J=2.3 Hz, 2H), 6.43-5.96 (m, 4H), 5.74-5.08 (m, 4H), 4.90 (s, 1H), 4.65-4.18 (m, 4H), 4.01-3.54 (m, 6H), 3.50-2.82 (m, 14H), 2.76-1.69 (m, 14H), 1.56-1.19 (m, 16H), 1.16-0.60 (m, 19H).

### Example 52: Synthesis of (1R,2R,4S)-4-((2R)-2-((3S,6R,7E,9R,10R,12R,14S,15E,17E,19E,23S,26R,27R,34aS)-27-hydroxy-9,10-dimethoxy-6,8,12,14,20,26-hexamethyl-1,5,11,28,29-pentaoxo-21-(2-((tetrahydro-2H-pyran-4-yl)oxy)ethoxy)-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)-2-methoxycyclohexyl dimethylphosphinate (I-70), (1R,9S,12S,15R,16E,18R,19R,21R,23S,24E,26E,28E,30S,32S,35R)-12-[(1R)-2-[(1S,3R,4R)-4-dimethylphosphoryloxy-3-methoxy-cyclohexyl]-1-methyl-ethyl]-1-hydroxy-18,19-dimethoxy-15,17,21,23,29,35-hexamethyl-30-(2-tetrahydropyran-4-yloxyethoxy)-11,36-dioxa-4-azatricyclo[30.3.1.0^{4,9}]hexatriaconta-16,24,26,28-tetraene-2,3,10,14,20-pentone (I-55) and (1R,9S,12S,15R,16E,18R,19R,21R,23S,24E,26E,28E,30R,32S,35R)-12-[(1R)-2-[(1S,3R,4R)-4-dimethylphosphoryloxy-3-methoxy-cyclohexyl]-1-methyl-ethyl]-1-hydroxy-18,19-dimethoxy-15,17,21,23,29,35-hexamethyl-30-(2-tetrahydropyran-4-yloxyethoxy)-11,36-dioxa-4-azatricyclo[30.3.1.0^{4,9}]hexatriaconta-16,24,26,28-tetraene-2,3,10,14,20-pentone (I-54)

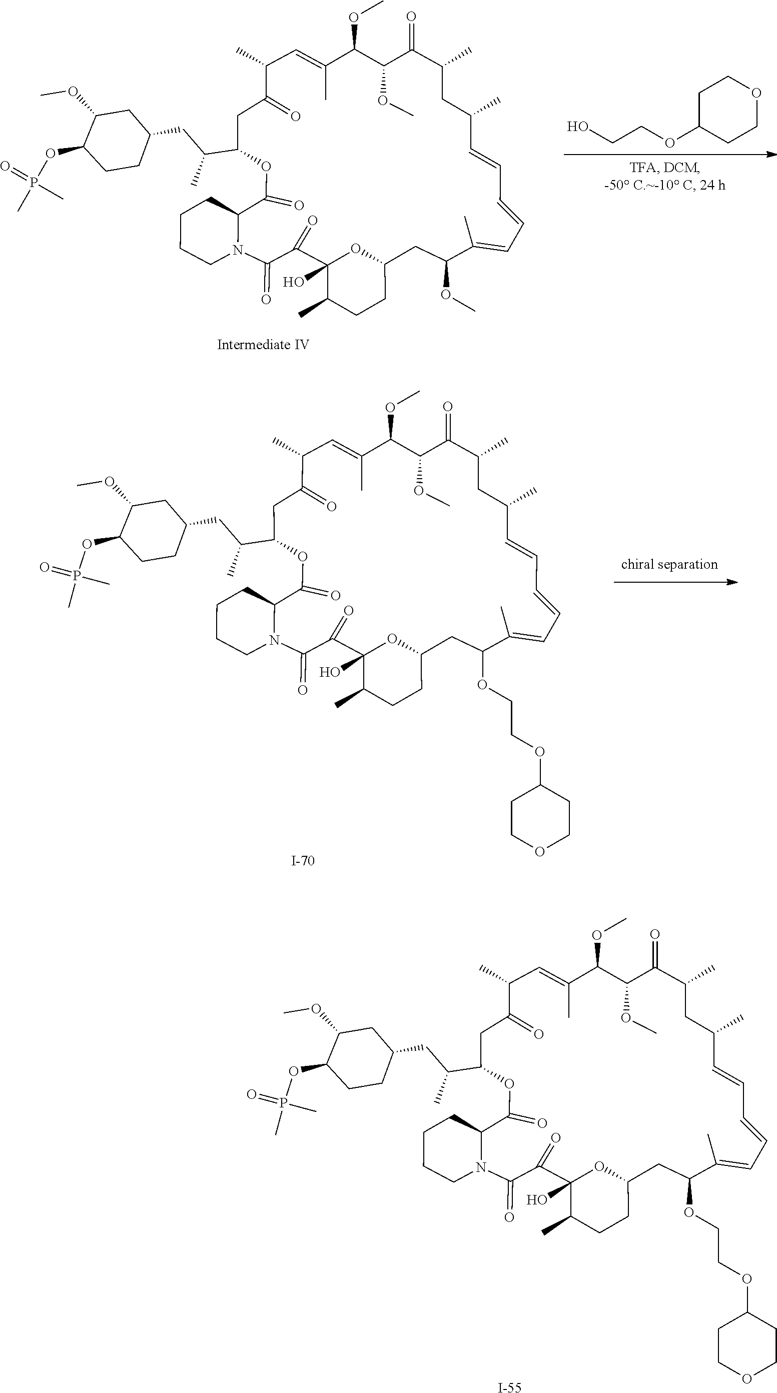

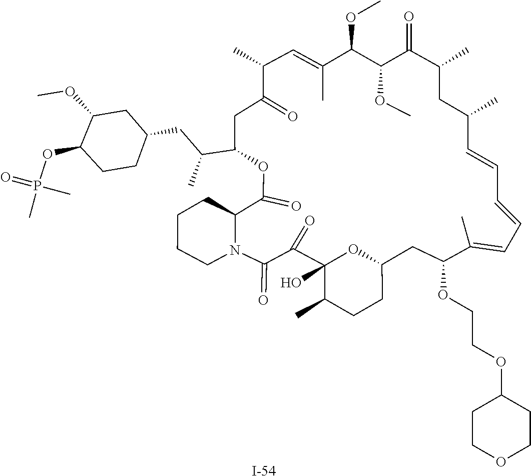

Step 1: (1R,2R,4S)-4-((2R)-2-((3 S,6R,7E,9R,10R,12R,14S,15E,17E,19E,23 S,26R,27R,34aS)-27-hydroxy-9,10-dimethoxy-6,8,12,14,20,26-hexamethyl-1,5,11,28,29-pentaoxo-21-(2-((tetrahydro-2H-pyran-4-yl)oxy)ethoxy)-1,4,5,6,9,10,11,12,13,14,21,22,23,24,25,26,27,28,29,31,32,33,34,34a-tetracosahydro-3H-23,27-epoxypyrido[2,1-c] [1]oxa[4]azacyclohentriacontin-3-yl)propyl)-2-methoxycyclohexyl dimethylphosphinate (I-70). To a solution of Intermediate IV (0.5 g, 0.498 mmol) in DCM (15 mL) was added TFA (2.27 g, 19.92 mmol, 1.53 mL) at −50° C. The reaction was stirred for 10 minutes then 2-tetrahydropyran-4-yloxyethanol (2.18 g, 14.94 mmol) dissolved in DCM (0.5 mL) was added. The mixture was stirred at −10° C. for 24 hr then diluted with DCM and NaHCO₃ aqueous solution. The organic layer was washed with water and brine, dried over Na₂SO₄, filtered and concentrated. The residue was purified via reverse-phase chromatography to provide I-70 (89.8 mg, 16% yield) as a white solid. ESI-MS (EI⁺, m/z): 1140.5 [M+Na]⁺. ¹H NMR (500 MHz, CDCl₃) δ 6.61-5.89 (m, 4H), 5.77-5.06 (m, 4H), 4.66-4.01 (m, 2H), 4.01-3.47 (m, 6H), 3.47-2.93 (m, 15H), 2.92-2.33 (m, 3H), 2.33-1.84 (m, 7H), 1.71-1.34 (m, 29H), 1.33-0.66 (m, 26H).

Step 2: (1R,9S,12S,15R,16E,18R,19R,21R,23S,24E,26E,28E,30S,32S,35R)-12-[(1R)-2-[(1S,3R,4R)-4-dimethylphosphoryloxy-3-methoxy-cyclohexyl]-1-methyl-ethyl]-1-hydroxy-18,19-dimethoxy-15,17,21,23,29,35-hexamethyl-30-(2-tetrahydropyran-4-yloxyethoxy)-11,36-dioxa-4-azatricyclo[30.3.1.0^{4,9}]hexatriaconta-16,24,26,28-tetraene-2,3,10,14,20-pentone (I-55) and (1R,9S,12S,15R,16E,18R,19R,21R,23S,24E,26E,28E,30R,32S,35R)-12-[(1R)-2-[(1S,3R,4R)-4-dimethylphosphoryloxy-3-methoxy-cyclohexyl]-1-methyl-ethyl]-1-hydroxy-18,19-dimethoxy-15,17,21,23,29,35-hexamethyl-30-(2-tetrahydropyran-4-yloxyethoxy)-11,36-dioxa-4-azatricyclo[30.3.1.0^{4,9}]hexatriaconta-16,24,26,28-tetraene-2,3,10,14,20-pentone (I-54). 100 mg of I-70 was separated via chiral preparatory HPLC and then purified via silica gel chromatography (8% MeOH in PE:DCM:EA 3:3:1) to provide I-55 (26.5 mg, 25% yield) as a white solid and I-54 (11.4 mg, 11% yield) as a white solid.

Chiral analysis method:

Column: CHIRALPAK IC (IC00CD-TB016)

Column size: 0.46 cm I.D.×15 cm L

Injection: 50 μl

Mobile phase: EtOH=100%

Flow rate: 0.5 mL/min

Wavelength: UV 254 nm

Temperature: 35° C.

HPLC equipment: Shimadzu LC-20AT

I-55: ESI-MS (EI⁺, m/z): 1140.6 [M+Na]⁺. ¹H NMR (500 MHz, CDCl₃) δ 6.50-5.76 (m, 4H), 5.58-4.96 (m, 4H), 4.19-3.49 (m, 9H), 3.46-2.86 (m, 17H), 2.81-2.46 (m, 2H), 2.37-1.68 (m, 18H), 1.60-1.17 (m, 22H), 1.13-0.70 (m, 20H).
I-54: ESI-MS (EI⁺, m/z): 1140.4 [M+Na]⁺. ¹H NMR (500 MHz, CDCl₃) δ 6.36-5.75 (m, 4H), 5.46-4.97 (m, 4H), 4.65 (s, 1H), 4.11 (d, J=5.7 Hz, 1H), 3.90-3.58 (m, 5H), 3.53-3.17 (m, 18H), 2.89-2.38 (m, 6H), 2.31-1.64 (m, 20H), 1.55-1.20 (m, 17H), 1.07-0.56 (m, 20H).

### Example 53: Synthesis of (1R,9S,12S,15R,16E,18R,19R,21R,23S,24E,26E,28E,32S,35R)-12-[(1R)-2-[(1S,3R,4R)-4-dimethylphosphoryloxy-3-methoxy-cyclohexyl]-1-methyl-ethyl]-1-hydroxy-18,19-dimethoxy-15,17,21,23,29,35-hexamethyl-30-[2-[2-(oxetan-3-yloxy)ethoxy]ethoxy]-11,36-dioxa-4-azatricyclo[30.3.1.04,9]hexatriaconta-16,24,26,28-tetraene-2,3,10,14,20-pentone (I-73)

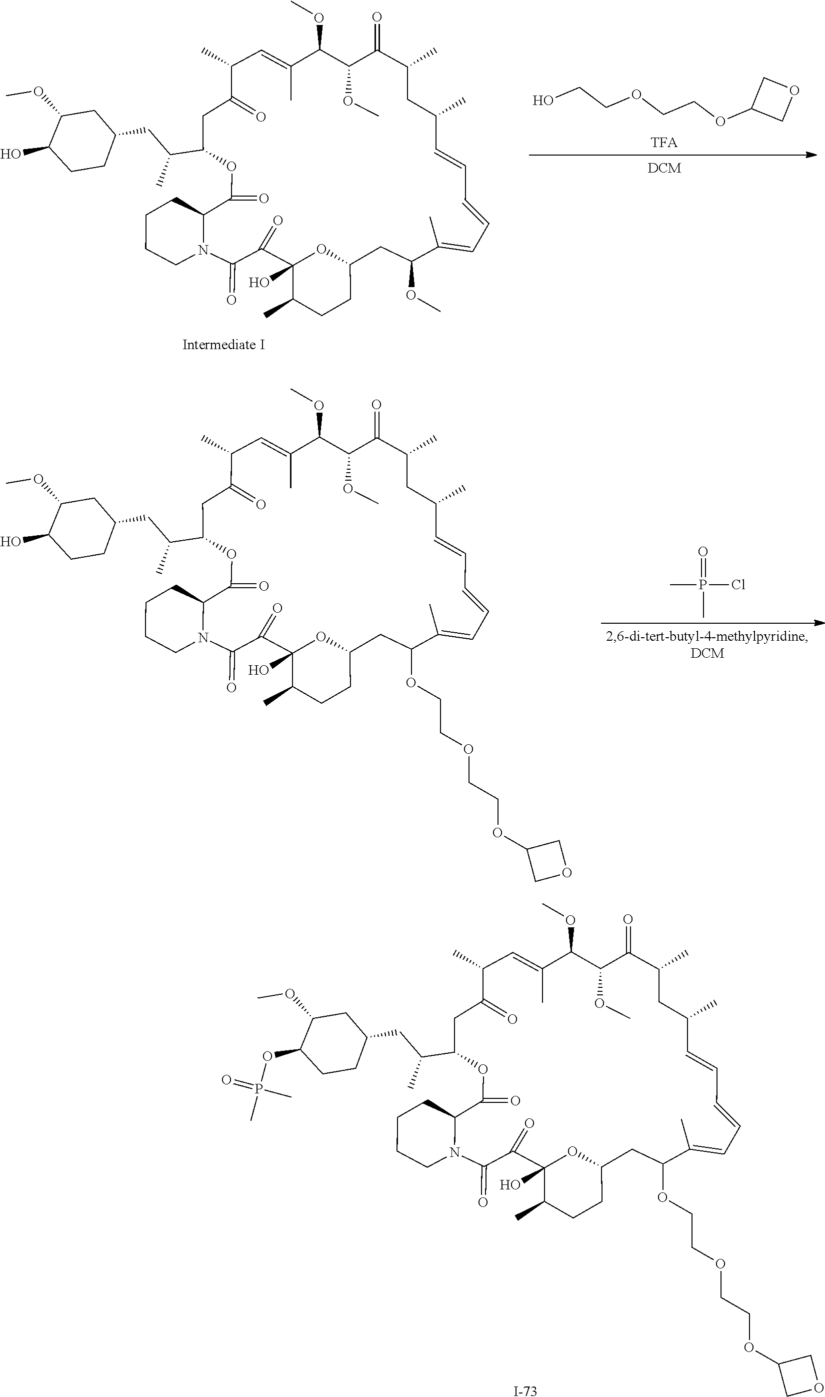

Step 1: (24E,26E,28E,29E,35R,36S,37R,38R,40S,42S,45S,47R,48R,57R)-57-hydroxy-45-[(1R)-2-[(1S,3R,4R)-4-hydroxy-3-methoxy-cyclohexyl]-1-methyl-ethyl]-47,48-dimethoxy-35,36,37,38,49,50-hexamethyl-44-[2-[2-(oxetan-3-yloxy)ethoxy]ethoxy]-67,68-dioxa-58-azatricyclohexatriaconta-24,26,28(49),29(50)-tetraene-51,52,53,54,55-pentone. To a solution of Intermediate I (0.5 g, 0.539 mmol) in DCM (5 mL) was added 2,2,2-trifluoroacetic acid (1.23 g, 10.77 mmol, 0.83 mL) dropwise at −55° C. under N₂. After stirring for 10 min at −45° C. 2-[2-(oxetan-3-yloxy)ethoxy]ethanol (1.75 g, 10.77 mmol in DCM) was added and the mixture was warmed to 0° C. over 1 h then poured into saturated aqueous NaHCO₃ (70 mL) at 0° C. and extracted with DCM (70 mL). The organic layer was washed with water (70 mL) and brine (70 mL), dried over anhydrous sodium sulfate, filtered and the filtrate was concentrated. The residue was purified by silica gel chromatography (100% EA), then by reverse-phase chromatography eluting with 60% CH₃CN in water to provide (24E,26E,28E,29E,35R,36S,37R,38R,40S,42S,45S,47R,48R,57R)-57-hydroxy-45-[(1R)-2-[(1S,3R,4R)-4-hydroxy-3-methoxy-cyclohexyl]-1-methyl-ethyl]-47,48-dimethoxy-35,36,37,38,49,50-hexamethyl-44-[2-[2-(oxetan-3-yloxy)ethoxy]ethoxy]-67,68-dioxa-58-azatricyclohexatriaconta-24,26,28(49),29(50)-tetraene-51,52,53,54,55-pentone (120 mg, 21% yield) as a white solid.

Step 2: (1R,9S,12S,15R,16E,18R,19R,21R,23S,24E,26E,28E,32S,35R)-12-[(1R)-2-[(1S,3R,4R)-4-dimethylphosphoryloxy-3-methoxy-cyclohexyl]-1-methyl-ethyl]-1-hydroxy-18,19-dimethoxy-15,17,21,23,29,35-hexamethyl-30-[2-[2-(oxetan-3-yloxy)ethoxy]ethoxy]-11,36-dioxa-4-azatricyclo[30.3.1.04,9]hexatriaconta-16,24,26,28-tetraene-2,3,10,14,20-pentone (I-73). To a solution of (1R,9S,12S,15R,16E,18R,19R,21R,23S,24E,26E,28E,32S,35R)-1-hydroxy-12-[(1R)-2-[(1S,3R,4R)-4-hydroxy-3-methoxy-cyclohexyl]-1-methyl-ethyl]-18,19-dimethoxy-15,17,21,23,29,35-hexamethyl-30-[2-[2-(oxetan-3-yloxy)ethoxy]ethoxy]-11,36-dioxa-4-azatricyclo[30.3.1.04,9]hexatriaconta-16,24,26,28-tetraene-2,3,10,14,20-pentone (0.3 g, 0.28 mmol) in DCM (6 mL) was added 2,6-di-tert-butyl-4-methylpyridine (0.437 g, 2.13 mmol) and dimethylphosphinic chloride (159.45 mg, 1.42 mmol) at 0° C. The reaction was stirred at 0° C. for 3.5 hr then diluted with EtOAc, washed with NaHCO₃ aqueous solution, washed with water, brine, dried over Na₂SO₄, filtered and concentrated. The residue was purified by reverse-phase chromatography (CH₃CN in water from 0% to 75%) to provide I-73 (110 mg, 34% yield) as a white solid. ESI-MS (EI⁺, m/z): 1156.6 [M+Na]⁺.

### Example 54: Synthesis of (1R,9S,12S,15R,16E,18R,19R,21R,23S,24E,26E,28E,32S,35R)-30-[2-[2-(dimethylamino)ethoxy]ethoxy]-1-hydroxy-12-[(1R)-2-[(1S,3R,4R)-4-(2-hydroxyethoxy)-3-methoxy-cyclohexyl]-1-methyl-ethyl]-18,19-dimethoxy-15,17,21,23,29,35-hexamethyl-11,36-dioxa-4-azatricyclo[30.3.1.04,9]hexatriaconta-16,24,26,28-tetraene-2,3,10,14,20-pentone (I-53)

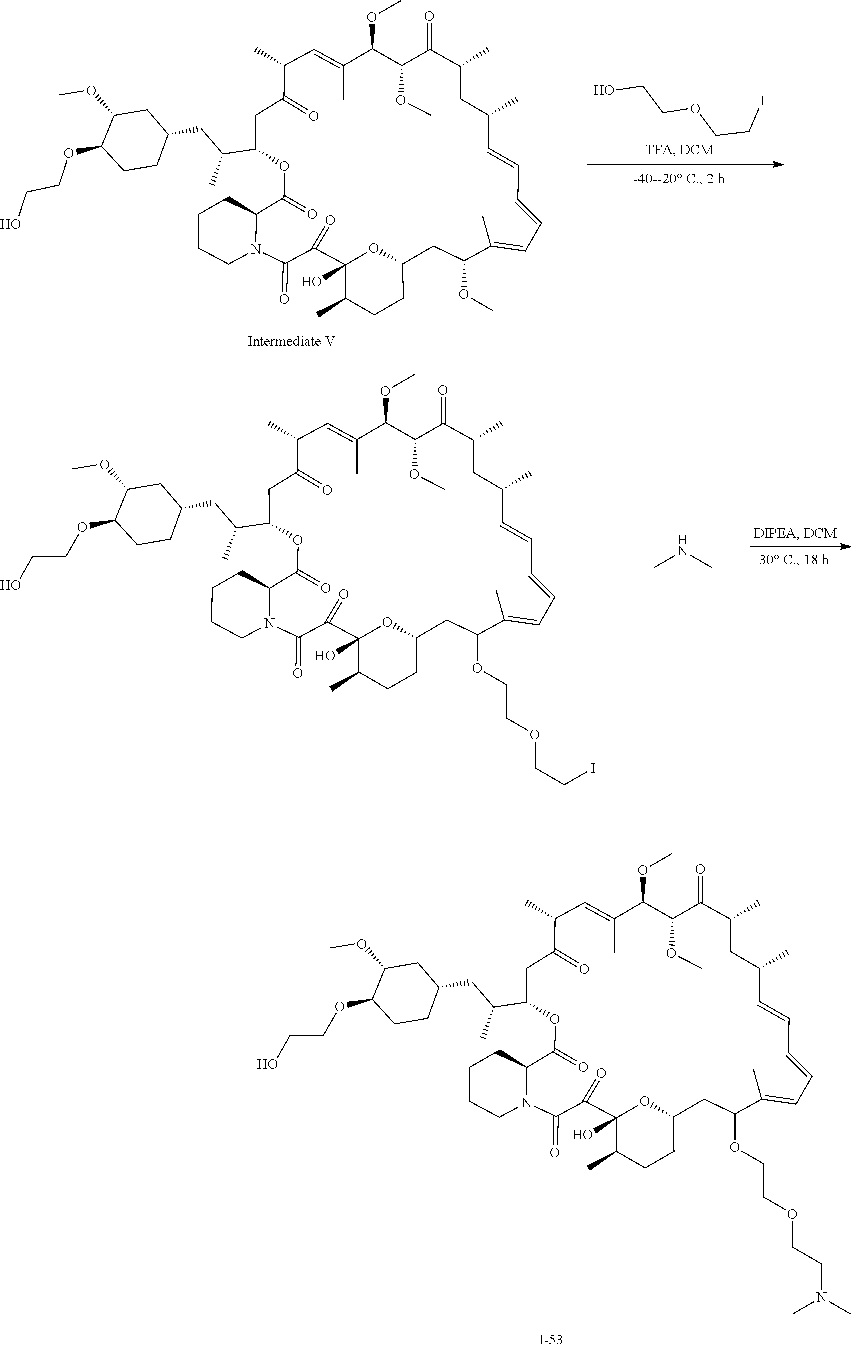

Step 1: (1R,9S,12S,15R,16E,18R,19R,21R,23S,24E,26E,28E,32S,35R)-1-hydroxy-12-[(1R)-2-[(1S,3R,4R)-4-(2-hydroxyethoxy)-3-methoxy-cyclohexyl]-1-methyl-ethyl]-30-[2-(2-iodoethoxy)ethoxy]-18,19-dimethoxy-15,17,21,23,29,35-hexamethyl-11,36-dioxa-4-azatricyclo[30.3.1.04,9]hexatriaconta-16,24,26,28-tetraene-2,3,10,14,20-pentone. To a solution of Intermediate V (0.24 g, 0.247 mmol) in DCM (10 mL) was added TFA (844.38 mg, 7.41 mmol, 0.57 mL) at −50° C. After 10 minutes, 2-(2-iodoethoxy)ethanol (1.07 g, 4.94 mmol) in DCM (0.05 mL) was added and the mixture was stirred at −20° C. for 5 h. The reaction was diluted with DCM and aqueous NaHCO₃ solution, and the organic layer was washed with water and brine, dried over Na₂SO₄, filtered and concentrated. The residue was purified via reverse-phase chromatography to provide (1R,9S,12S,15R,16E,18R,19R,21R,23S,24E,26E,28E,32S,35R)-1-hydroxy-12-[(1R)-2-[(1S,3R,4R)-4-(2-hydroxyethoxy)-3-methoxy-cyclohexyl]-1-methyl-ethyl]-30-[2-(2-iodoethoxy)ethoxy]-18,19-dimethoxy-15,17,21,23,29,35-hexamethyl-11,36-dioxa-4-azatricyclo[30.3.1.04,9]hexatriaconta-16,24,26,28-tetraene-2,3,10,14,20-pentone (130 mg, 46% yield) as a white solid.

Step 2: (1R,9S,12S,15R,16E,18R,19R,21R,23S,24E,26E,28E,32S,35R)-30-[2-[2-(dimethylamino)ethoxy]ethoxy]-1-hydroxy-12-[(1R)-2-[(1S,3R,4R)-4-(2-hydroxyethoxy)-3-methoxy-cyclohexyl]-1-methyl-ethyl]-18,19-dimethoxy-15,17,21,23,29,35-hexamethyl-11,36-dioxa-4-azatricyclo[30.3.1.04,9]hexatriaconta-16,24,26,28-tetraene-2,3,10,14,20-pentone (I-53). A solution of (1R,9S,12S,15R,16E,18R,19R,21R,23S,24E,26E,28E,32S,35R)-1-hydroxy-12-[(1R)-2-[(1S,3R,4R)-4-(2-hydroxyethoxy)-3-methoxy-cyclohexyl]-1-methyl-ethyl]-30-[2-(2-iodoethoxy)ethoxy]-18,19-dimethoxy-15,17,21,23,29,35-hexamethyl-11,36-dioxa-4-azatricyclo[30.3.1.04,9]hexatriaconta-16,24,26,28-tetraene-2,3,10,14,20-pentone (0.36 g, 0.31 mmol), N-methylmethanamine (0.42 g, 9.34 mmol, 0.54 mL) and N-ethyl-N-isopropyl-propan-2-amine (1.21 g, 9.34 mmol, 1.63 mL) in DCM (3.92 mL) was stirred for 17 h at 25° C. The reaction mixture was diluted with DCM (10 mL) and washed with saturated NH₄Cl (10 mL×3), water (10 mL×3) and brine (10 mL×3), dried over anhydrous sodium sulfate, filtered and the filtrate was concentrated in vacuo. The residue was purified by reverse-phase column eluting with 50% CH₃CN in water to provide I-53 (40 mg, 12% yield) as a yellow solid. ESI-MS (EI⁺, m/z): 1095.4 [M+Na]⁺.

### Example 55: AlphaLISA Ultra pS6K1 Assay

Assay Protocol:
1. Seed MCF-7 cells in Corning 3701 plate and incubate for 20˜24 hour. 12,000˜16,000 cells will be seeded in 36 μL medium per well.
2. Change the culture medium with fresh medium and incubate for another 2 hours.
3. Add 12 μL (4×) compounds into the cell plate by HAMILTON. Final DMSO concentration is 0.5%. Incubate for 90 minutes.
4. Aspirate 38 μL by HAMILTON, 10 μL rest per well.
5. Add 10 μL 2× lysis buffer using HAMILTON; total volume in wells is 20 μL. Allow cells to shake for 30 min. Cover plate by plastic foil and store plate at −80° C. up to analysis.
6. Thaw cell lysate at RT and transfer 10 μL lysate to assay plate (Optiplate-384).
7. Add 5 μL acceptor beads into assay plate and incubation for 2 hours
8. Add 5 μL donor beads and incubation for 2 hours
9. Count the plate by EnSpire Multimode Plate Reader

TABLE 2
Key Reagents/Supplies

| Reagents/materials | Vendor | Cat. No. | Lot. No. |
|---|---|---|---|
| MCF-7 | ATCC | HTB-22 | 5105360 |
| DMEM | Invitrogen | 12430-054 | 1677193 |
| FBS | Invitrogen | 10099-141 | 1660516 |
| 0.25% Trypsin-EDTA | Invitrogen | 25200-072 | 1638603 |
| 384 well plate, tissue culture | Corning | CLS3701 | 29214010 |
| treated | | | |
| Corning 384 well storage | Corning | CLS3656 | 29514036 |
| plates | | | |
| Torin1 | Selleck | S2827 | 01 |
| OptiPlate-384, White Opaque | PerkinElmer | 6007299 | 8210-14501 |
| 384-well MicroPlate | | | |
| AlphaLISA SureFire Ultra | PerkinElmer | ALSU-PP70- | U0381 |
| p-p70 S6 Kinase (Thr389) | | A10K | |
| Assay Kit | | | |

### Example 56: AlphaLISA Ultra pAKT Assay

Assay Protocol:
1. MCF-7 cells in Corning 3701 plate and incubate for 20˜24 hour. 16,000˜20,000 cells will be seeded in 36 μL medium per well.
2. Change the culture medium with fresh medium and incubate for another 90 minutes.
3. Add 12 μL (4×) compounds into the cell plate by HAMILTON. Final DMSO concentration is 0.5%. Incubate for 2 hours.
4. Aspirate 38 μL by HAMILTON, 10 μL rest per well.
5. Add 10 μL 2× lysis buffer using HAMILTON; total volume in wells is 20 μL. Allow cells to shake for 30 min. Cover plate by plastic foil and store plate at −80° C. up to analysis.
6. Thaw cell lysate at RT and transfer 10 μL lysate to assay plate (Optiplate-384).
7. Add 5 μL acceptor beads into assay plate and incubation for 2 hours
8. Add 5 μL donor beads and incubation for 2 hours
9. Count the plate by EnSpire Multimode Plate Reader

TABLE 3
Key Reagents/Supplies

| Reagents/materials | Vendor | Cat. No. | Lot. No. |
|---|---|---|---|
| MCF-7 | ATCC | HTB-22 | 5105360 |
| DMEM | Invitrogen | 12430-054 | 1677193 |
| FBS | Invitrogen | 10099-141 | 1660516 |
| 0.25% Trypsin-EDTA | Invitrogen | 25200-072 | 1638603 |
| 384 well plate, tissue | Corning | CLS3701 | 29214010 |
| culture treated | | | |
| Corning 384 well storage | Corning | CLS3656 | 29514036 |
| plates | | | |
| Torin1 | Selleck | S2827 | 01 |
| OptiPlate-384, White | PerkinElmer | 6007299 | 8210-14501 |
| Opaque 384-well MicroPlate | | | |
| AlphaLISA SureFire Ultra | PerkinElmer | ALSU-PAKT- | U0329 |
| p-Akt 1/2/3 (Ser473) | | B10K | |
| Assay Kits | | | |

### Example 57: Western Blot Based pS6K1 and pAKT Assay at 24 and 48 Hour Timepoints

Assay Protocol:
1. Seed six well plate with 500,000 PC3 cells per well and incubate for 20˜24 hour.
2. Add compounds into the cell plate. Incubate for 24 to 48 hours.
4. Plate is placed on ice and the media is removed via aspiration. The wells are washed with 1 mL of 1x PBS and then fully aspirated.
5. 110 μL of 1% Triton Lysis Buffer is added and each well is scraped vigorously.
6. Cell homogenates are transferred to 1.5 mL eppendorf tubes on ice and spun down at 4° C. for 10 minutes at 10,000 rpm.
7. Protein concentration of resulting cell lysates were quantified utilizing a Bradford assay and the samples run analyzed via Western blot on 4-12% Bis/Tris gels with 1×MES buffer.
8. The gels were transferred onto membranes at 50V for 100 minutes, blocked with Odyssey Blocking buffer for 30 minutes then incubated overnight with primary antibody (pS6K1 T389 Rabbit or pAkt S473 Rabbit) overnight at 4° C. on a rotator.
9. The membranes were washed 3× with TBS-T with a 5 minute incubation between each wash then incubated with secondary antibody (LiCor IRDye 800 Donkey Anti Rabbit) for at least 30 minutes.
10. The membranes were washed 3× with TBS-T with a 5 minute incubation between each wash.
11. The gels were then incubated for 5 minutes with PBS at room temperature then imaged using a Li-Cor.

Table 4 shows the inhibitory activity (IC₅₀) of selected compounds of this invention in the pS6K1 and pAKT assays, and their solubility in 100 mM phosphate buffer (pH 7.4). The compound numbers correspond to the compound numbers in Table 1.

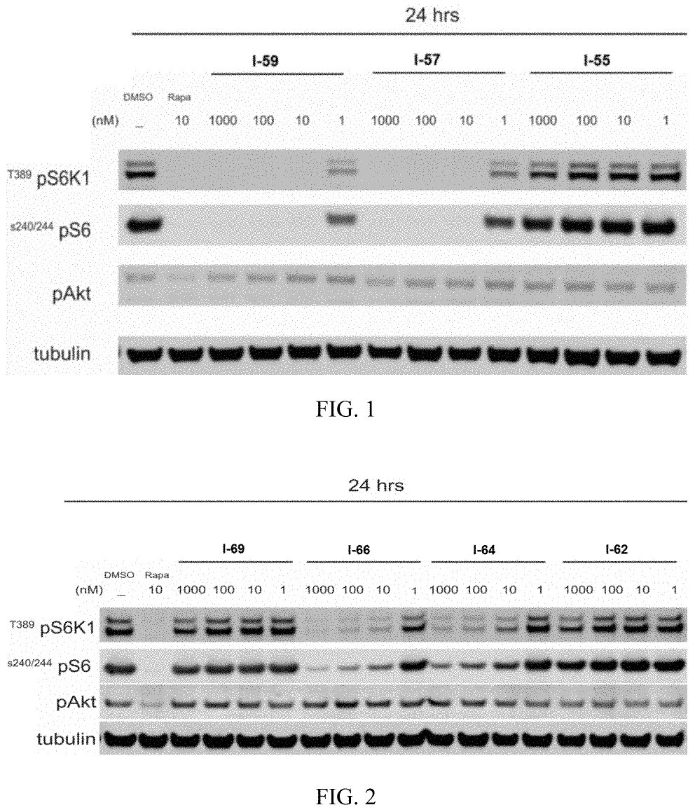

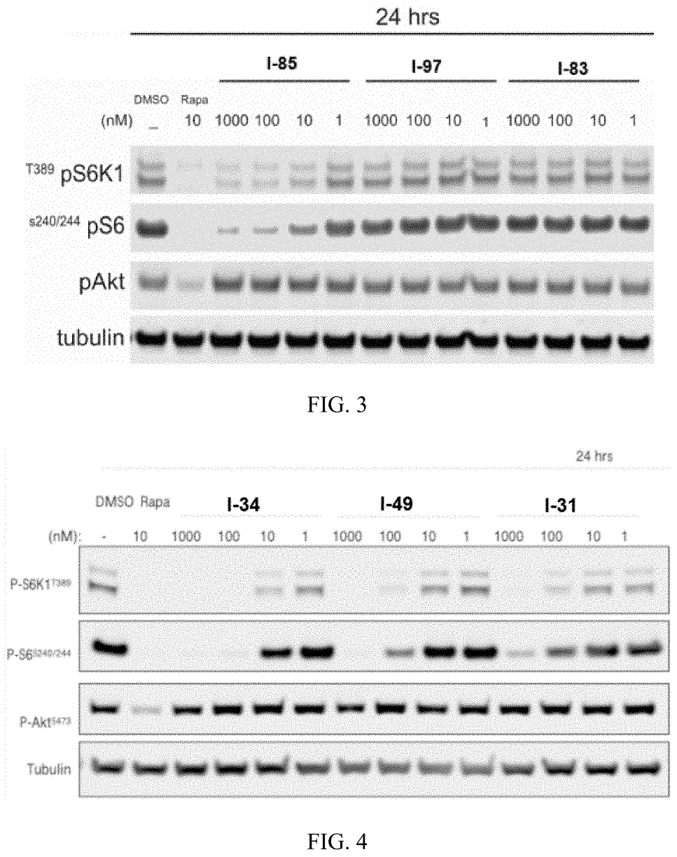

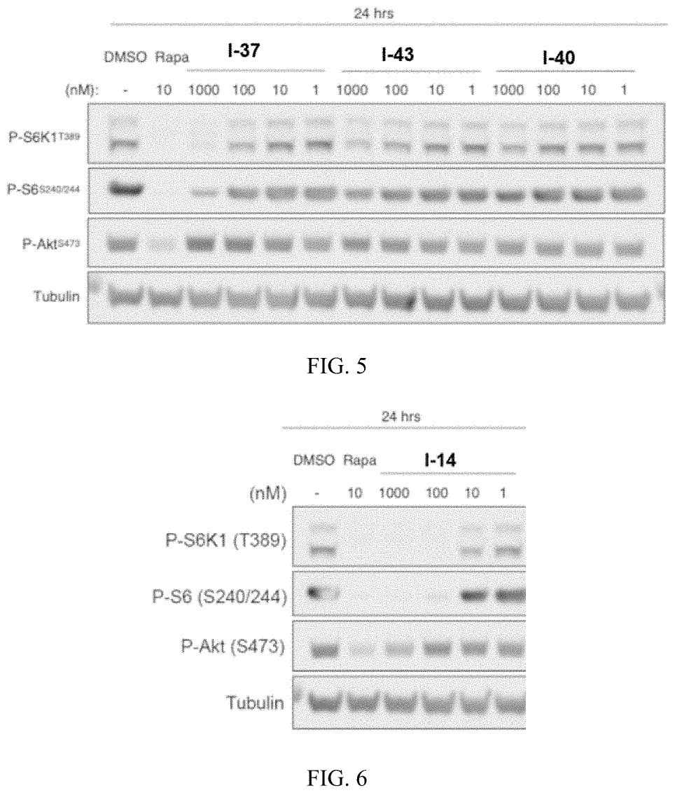

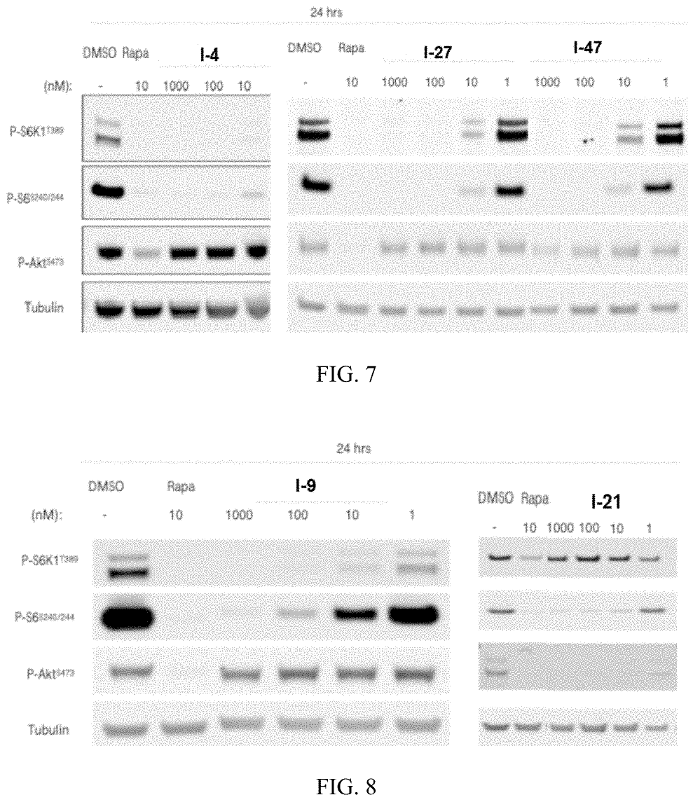

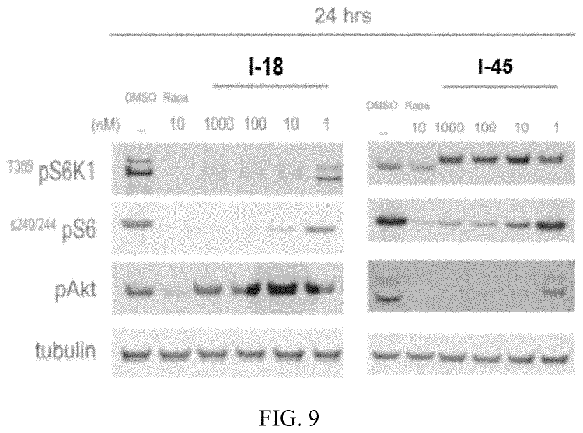

Compounds of the present invention that selectively inhibit mTORC1 over mTORC2 by comparing pS6K1 and pAKT IC₅₀ by kinase assays are indicated by “YES” in the “mTORC1 selective @ 90 min” column of Table 4. Compounds that are not selective by comparing pS6K1 and pAKT IC₅₀ by kinase assays are indicated by “NO” in the “mTORC1 selective @ 90 min” column of Table 4. Compounds of the present invention that selectively inhibit mTORC1 over mTORC2 by Western Blot assays and retain selectivity for at least 24 hours—are indicated by “YES” in the “mTORC1 selective @ 24 hrs” column of Table 4 and images of Western Blot assays as illustrated in FIGS. 1-9. Compounds that are not selective at the 24 hrs mark are indicated by “NO” in the “mTORC1 selective @ 24 hrs” column of Table 4. “N/A” stands for “not assayed” and “N/C” stands for “not calculated”.

Compounds denoted “A” exhibited an IC₅₀ lower than 1 nM (x<1 nM). Compounds denoted “B” exhibited an IC₅₀ greater than or equal to 1 nM and less than 10 nM (1 nM≤x<10 nM). Compounds denoted “C” exhibited an IC₅₀ greater than or equal to 10 nM and less than 100 nM (10 nM≤x<100 nM). Compounds denoted “D” exhibited an IC₅₀ greater than or equal to 100 nM and less than 1 μM (100 nM≤x<1 μM). Compounds denoted “E” exhibited an IC₅₀ greater than or equal to 1 μM (1 μM≤x).

TABLE 4
Assay Data for Exemplary Compounds

| | pS6K1 in | pAKT in | mTORC1 | mTORC1 |
|---|---|---|---|---|
| | MCF7 @ 90 | MCF7 @ 90 | selective @ | selective @ |
| I-# | min (IC₅₀) | min (IC₅₀) | 90 min | 24 hrs |
| I-4 | B | N/A | — | Yes |
| I-5 | C | E | Yes | N/A |
| I-6 | C | E | Yes | N/A |
| I-7 | B | E | Yes | N/A |
| I-8 | C | E | Yes | N/A |
| I-9 | B | N/A | — | Yes |
| I-10 | C | E | Yes | N/A |
| I-11 | C | E | Yes | N/A |
| I-12 | A | E | Yes | N/A |
| I-13 | B | E | Yes | N/A |
| I-14 | A | N/A | — | Yes |
| I-15 | B | E | Yes | N/A |
| I-17 | B | E | Yes | N/A |
| I-18 | N/A | N/A | — | Yes |
| I-19 | A | E | Yes | N/A |
| I-20 | B | E | Yes | N/A |
| I-21 | A | N/A | — | Yes |
| I-22 | D | E | Yes | N/A |
| I-23 | D | E | Yes | N/A |
| I-24 | N/C | E | — | N/A |
| I-25 | C | E | Yes | N/A |
| I-27 | C | N/A | — | Yes |
| I-30 | C | E | Yes | N/A |
| I-31 | C | N/A | — | Yes |
| I-33 | B | E | Yes | N/A |
| I-34 | B | N/A | — | Yes |
| I-36 | B | E | Yes | N/A |
| I-37 | B | N/A | — | Yes |
| I-39 | D | E | Yes | N/A |
| I-40 | E | N/A | — | Yes |
| I-42 | C | E | Yes | N/A |
| I-43 | C | N/A | — | Yes |
| I-44 | B | E | Yes | N/A |
| I-45 | A | N/A | — | Yes |
| I-47 | B | N/A | — | Yes |
| I-48 | B | E | Yes | N/A |
| I-49 | B | N/A | — | Yes |
| I-50 | B | E | Yes | N/A |
| I-55 | N/A | N/A | — | No |
| I-57 | N/A | N/A | — | Yes |
| I-59 | N/A | N/A | — | Yes |
| I-62 | D | N/A | — | No |
| I-63 | B | N/A | — | N/A |
| I-64 | B | N/A | — | Yes |
| I-65 | B | N/A | — | N/A |
| I-66 | B | N/A | — | Yes |
| I-67 | A | N/A | — | N/A |
| I-68 | A | N/A | — | N/A |
| I-69 | E | N/A | — | No |
| I-71 | B | N/A | — | N/A |
| I-72 | A | N/A | — | N/A |
| I-73 | C | N/A | — | N/A |
| I-74 | N/C | N/A | — | N/A |
| I-75 | E | E | Yes | N/A |
| I-76 | A | E | Yes | N/A |
| I-77 | C | E | Yes | N/A |
| I-78 | A | E | Yes | N/A |
| I-79 | B | E | Yes | N/A |
| I-80 | C | E | Yes | N/A |
| I-81 | C | E | Yes | N/A |
| I-82 | C | E | Yes | N/A |
| I-83 | E | E | No | No |
| I-84 | B | E | Yes | N/A |
| I-85 | B | E | Yes | Yes |
| I-86 | A | E | Yes | N/A |
| I-87 | N/C | E | — | N/A |
| I-88 | B | E | Yes | N/A |
| I-89 | B | E | Yes | N/A |
| I-90 | C | E | Yes | N/A |
| I-91 | B | E | Yes | N/A |
| I-92 | C | E | Yes | N/A |
| I-93 | B | E | Yes | N/A |
| I-94 | C | E | Yes | N/A |
| I-95 | D | E | Yes | N/A |
| I-96 | E | E | No | N/A |
| I-97 | E | E | No | No |
| I-99 | B | E | Yes | N/A |
| I-100 | B | E | Yes | N/A |
| I-101 | C | E | Yes | N/A |
| I-102 | A | E | Yes | N/A |
| I-103 | B | E | Yes | N/A |
| I-104 | C | E | Yes | N/A |
| I-105 | B | E | Yes | N/A |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I′: 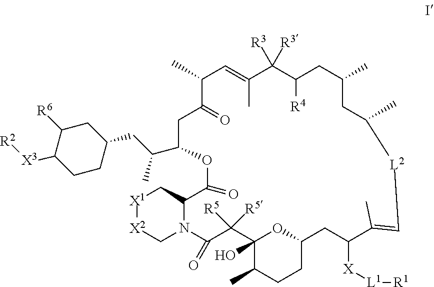
or a pharmaceutically acceptable salt thereof, wherein:
X and X³ are independently a covalent bond, —CR₂—, —NR—, —NRCO—, —NRCO₂—, —NRCONR—, —NRSO₂—, —O—, —S—, or —SO₂NR—;
L is a covalent bond or a C₁₋₃₀ bivalent straight or branched saturated or unsaturated hydrocarbon chain, wherein 1-10 methylene units of the chain are independently and optionally replaced with -Cy₁-, —O—, —S—, —SO₂—, —C(O)—, —C(S)—, —CR₂—, —CF₂—, —P(O)(R)—, —SiR₂—, —Si(OR)(R)—, or —NR—;
each -Cy₁- is independently an optionally substituted bivalent ring selected from phenylene, 4-7 membered saturated or partially unsaturated heterocyclylene having 1-2 heteroatoms independently selected from nitrogen, oxygen, and sulfur, and 5-6 membered heteroarylene having 1-4 heteroatoms independently selected from nitrogen, oxygen, and sulfur;
each R is independently hydrogen, or an optionally substituted group selected from C₁₋₆ aliphatic, 3-8 membered saturated or partially unsaturated monocyclic carbocyclic ring, phenyl, 4-7 membered saturated or partially unsaturated heterocyclic ring having 1-2 heteroatoms independently selected from nitrogen, oxygen, and sulfur, and 5-6 membered heteroaryl ring having 1-4 heteroatoms independently selected from nitrogen, oxygen, and sulfur, or
two R groups on the same atom are taken together with their intervening atoms to form a 4-7 membered saturated, partially unsaturated, or aryl ring having 0-3 heteroatoms, in addition to the same atom to which they are attached, independently selected from nitrogen, oxygen, and sulfur;
L² is an optionally substituted C₁₋₆ bivalent straight or branched saturated or unsaturated hydrocarbon chain, wherein 1-2 methylene units of the chain are independently and optionally replaced with -Cy₁-;
R¹ and R² are independently hydrogen, halogen, —OR, —CN, —(CR₂)₁₋₄NR₂, —COR, —CONR₂, —CONR(CR₂)₁₋₄NR₂, —NO₂, —NR₂, —NR(C₁₋₆ haloalkyl), —NRCOR, —NRCO₂R, —NRCONR₂, —NRSO₂R, —SR, —SO₂NR₂, —P(O)R₂, or an optionally substituted group selected from C₁₋₆ aliphatic, 3-8 membered saturated or partially unsaturated monocyclic carbocyclic ring, phenyl, 8-10 membered bicyclic aromatic carbocyclic ring, 4-8 membered saturated or partially unsaturated monocyclic heterocyclic ring having 1-2 heteroatoms independently selected from nitrogen, oxygen, and sulfur, 7-10 membered saturated or partially unsaturated bicyclic heterocyclic ring having 1-3 heteroatoms independently selected from nitrogen, oxygen, and sulfur, 5-6 membered monocyclic heteroaromatic ring having 1-4 heteroatoms independently selected from nitrogen, oxygen, and sulfurs, and 8-10 membered bicyclic heteroaromatic ring having 1-5 heteroatoms independently selected from nitrogen, oxygen, and sulfur;
R³ is hydrogen, halogen; —OR, or —OSiR₃;
R^{3′} is hydrogen, halogen; —OR, or —OSiR₃,
or R³ and R^{3′} are taken together to form ═O or ═S;
R⁴ and R⁶ are independently hydrogen, —OR, —NR₂, —NRCOR, —NRCO₂R, —NRCONR₂, —NRSO₂R, —SR, —SO₂NR₂, or an optionally substituted C₁₋₆ aliphatic;
R⁵ and R^{5′} are each hydrogen or taken together to form ═O or ═NOR;
X¹ and X² are each independently —CR₂—, —S—, or —S(O)—,
wherein at least one of X¹ and X² is —CR₂—.

2. The compound of claim 1, wherein said compound is selected from any one of the following formulae: 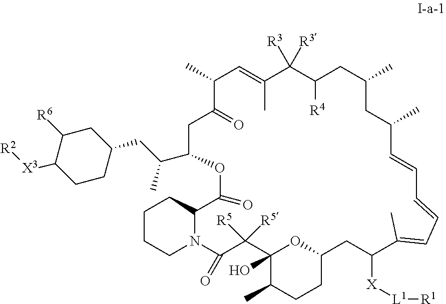
wherein:
X is —CR₂—, —NRCO—, —NRCO₂—, —NRCONR—, —NRSO₂—, or —SO₂NR—, 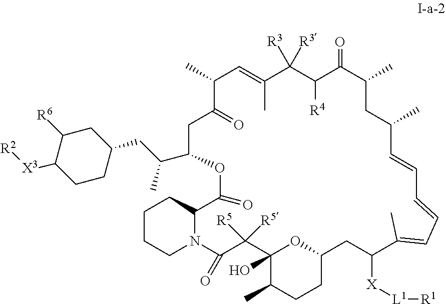
wherein:
X³ is —CR₂—, —NRCO—, —NRCO₂—, —NRCONR—, —NRSO₂—, or —SO₂NR—, 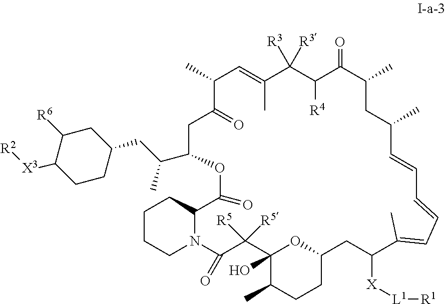
wherein:
R⁴ is —NR₂, —NRCOR, —NRCO₂R, —NRCONR₂, —NRSO₂R, —SR, —SO₂NR₂, or an optionally substituted C₁₋₆ aliphatic, 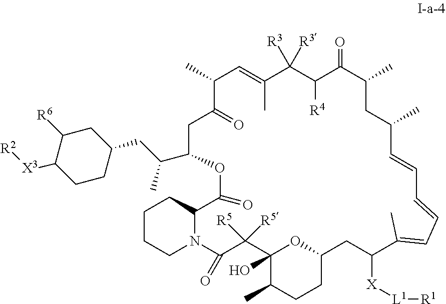
wherein:
R⁶ is —NR₂, —NRCOR, —NRCO₂R, —NRCONR₂, —NRSO₂R, —SR, —SO₂NR₂, or an optionally substituted C₁₋₆ aliphatic, 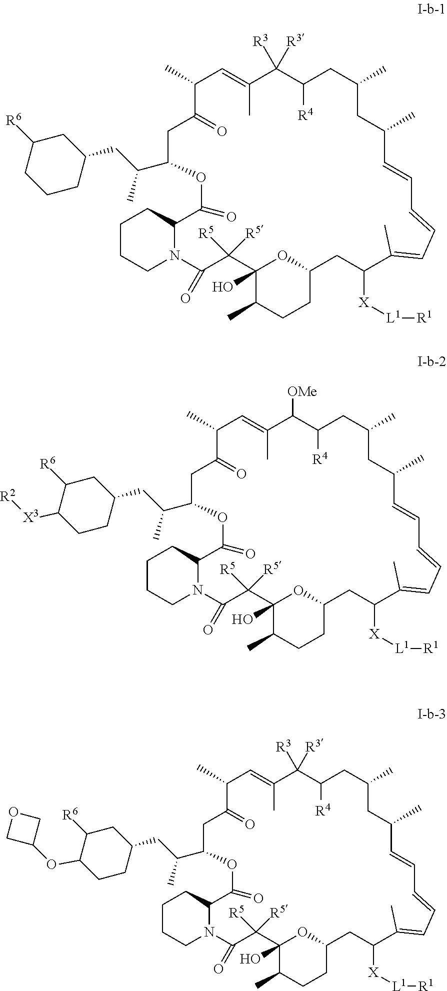
or a pharmaceutically acceptable salt thereof.

3. The compound of either of claim 1, wherein L¹ is a C₁₋₁₀ bivalent straight or branched saturated or unsaturated hydrocarbon chain wherein 1-5 methylene units of the chain are independently and optionally replaced with -Cy₁-, —O—, —S—, —SO₂—, —C(O)—, —C(S)—, —CR₂—, —CF₂—, —P(O)(R)—, or —NR—.

4. The compound of claim 1, wherein L¹ is selected from —CH₂—, —CH₂CH₂—, —(CH₂)₃—, —(CH₂)₄—, —(CH₂)₅—, —CH₂CH₂O—, —(CH₂CH₂O)₂—, —(CH₂CH₂O)₃—, —CH₂CH₂OCH₂CH₂—, —CH₂CH₂SO₂CH₂CH₂O—, and —CH₂CH₂OCH₂CH₂OCH₂CH₂—.

5. The compound of claim 1, wherein L¹ is a covalent bond.

6. The compound of claim 1, wherein each R is independently hydrogen, or an optionally substituted group selected from C₁₋₆ aliphatic, 3-8 membered saturated or partially unsaturated monocyclic carbocyclic ring, phenyl, 4-7 membered saturated or partially unsaturated heterocyclic ring having 1-2 heteroatoms independently selected from nitrogen, oxygen, and sulfur, and 5-6 membered heteroaryl ring having 1-4 heteroatoms independently selected from nitrogen, oxygen, and sulfur.

7. The compound of claim 1, wherein R¹ is selected from hydrogen, —OR, —CN, —NR₂, —NR(C₁₋₆ haloalkyl), —NRCOR, —NRCO₂R, —NRCONR₂, —NRSO₂R, —SR, —SO₂NR₂, or an optionally substituted group selected from C₁₋₆ aliphatic, 3-8 membered saturated or partially unsaturated monocyclic carbocyclic ring, phenyl, 8-10 membered bicyclic aromatic carbocyclic ring, 4-8 membered saturated or partially unsaturated monocyclic heterocyclic ring having 1-2 heteroatoms independently selected from nitrogen, oxygen, and sulfur, 7-10 membered saturated or partially unsaturated bicyclic heterocyclic ring having 1-3 heteroatoms independently selected from nitrogen, oxygen, and sulfur, 5-6 membered monocyclic heteroaromatic ring having 1-4 heteroatoms independently selected from nitrogen, oxygen, and sulfurs, and 8-10 membered bicyclic heteroaromatic ring having 1-5 heteroatoms independently selected from nitrogen, oxygen, and sulfur.

8. The compound of claim 1, wherein R¹ is methyl, —CH₂CF₃, —NH₂, —NHMe, —NMe₂, —SO₂NH₂, —CONH₂, —CONMe₂, —OCONHMe, —CO₂H,

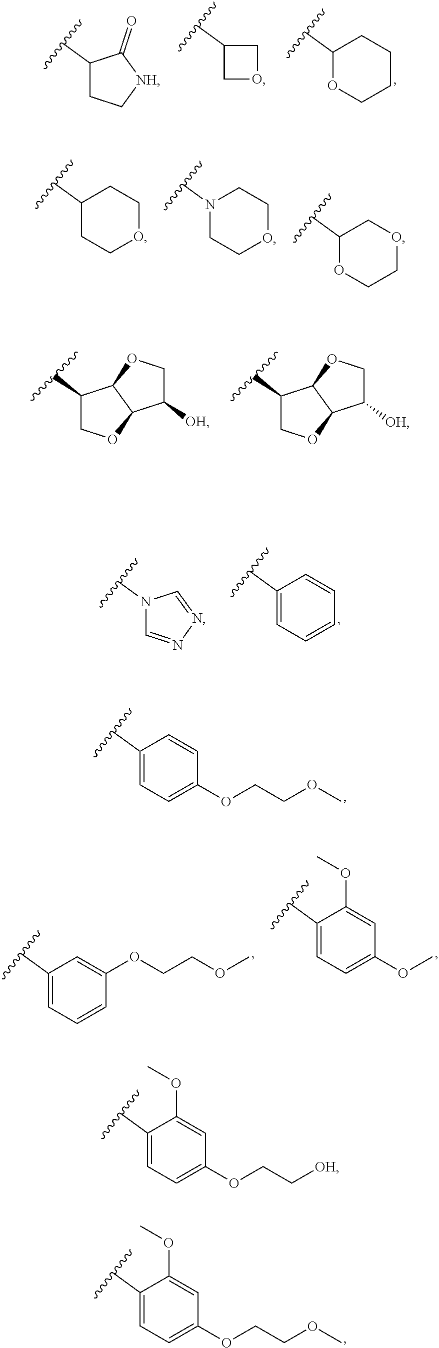

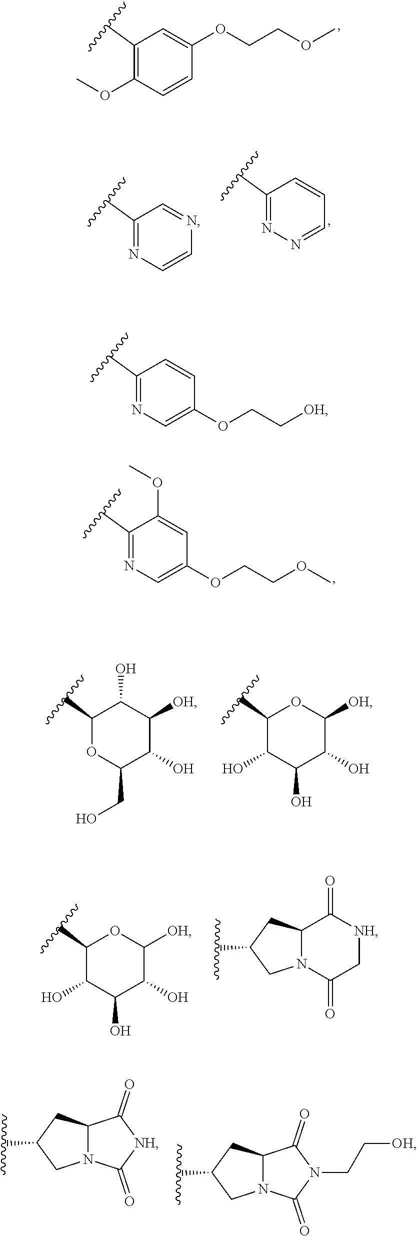

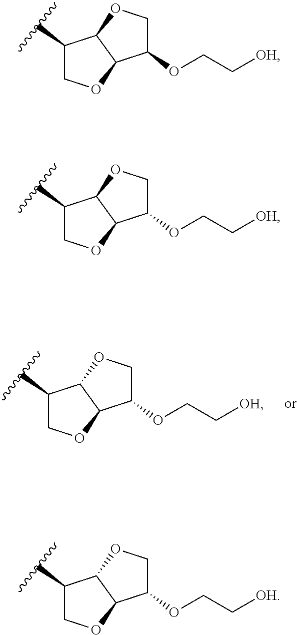 [SMILES: *[C@@H]1CO[C@H]2[C@@H]1OC[C@H]2OCCO ; *[C@@H]1CO[C@H]2[C@@H]1OC[C@@H]2OCCO ; *[C@@H]1CO[C@H]2[C@H]1OC[C@@H]2OCCO ; *[C@@H]1CO[C@H]2[C@H]1OC[C@@H]2OCCO]

9. The compound of claim 1, wherein R² is selected from hydrogen, —OR, —CN, —NR₂, —NR(C₁₋₆ haloalkyl), —NRCOR, —NRCO₂R, —NRCONR₂, —NRSO₂R, —SR, —SO₂NR₂, or an optionally substituted group selected from C₁₋₆ aliphatic, 3-8 membered saturated or partially unsaturated monocyclic carbocyclic ring, phenyl, 8-10 membered bicyclic aromatic carbocyclic ring, 4-8 membered saturated or partially unsaturated monocyclic heterocyclic ring having 1-2 heteroatoms independently selected from nitrogen, oxygen, and sulfur, 7-10 membered saturated or partially unsaturated bicyclic heterocyclic ring having 1-3 heteroatoms independently selected from nitrogen, oxygen, and sulfur, 5-6 membered monocyclic heteroaromatic ring having 1-4 heteroatoms independently selected from nitrogen, oxygen, and sulfurs, and 8-10 membered bicyclic heteroaromatic ring having 1-5 heteroatoms independently selected from nitrogen, oxygen, and sulfur.

10. The compound of claim 1, wherein R² is methyl, —CHF₂,

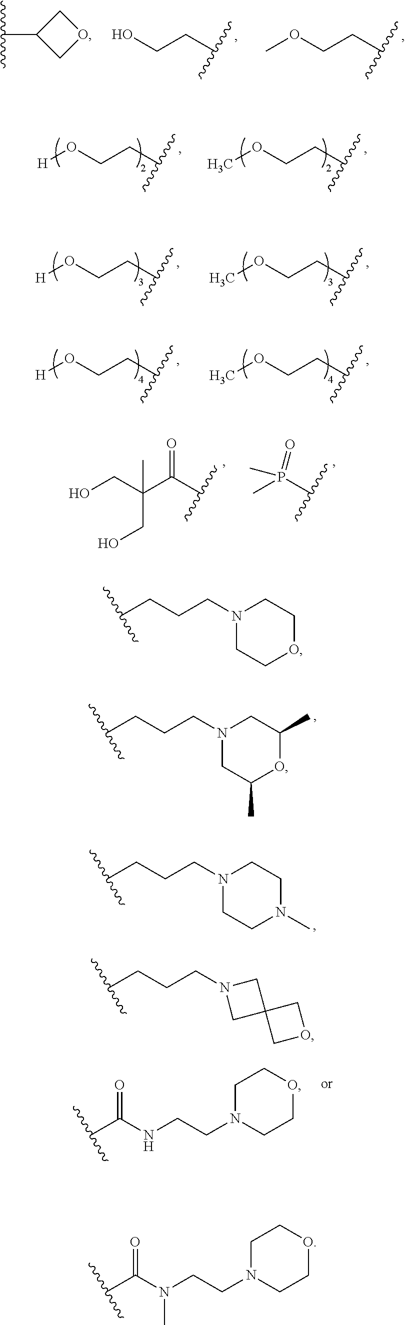

11. The compound of claim 1, wherein said compound is selected from those depicted in Table 1.

12. A pharmaceutically acceptable composition comprising a compound of claim 1, and a pharmaceutically acceptable carrier, adjuvant, or vehicle.

13. A method of treating an mTORC-mediated disease, disorder, or condition in a patient in need thereof, comprising administering to said patient the compound of claim 1, or a pharmaceutical composition thereof.

14. The method according to claim 13, further comprising administering an additional therapeutic agent in combination with said compound.

15. The method according to claim 13, wherein the mTORC-mediated disease, disorder, or condition is selected from diabetic nephropathy, kidney-related complications of type 1 diabetes and type 2 diabetes, autosomal dominant polycystic kidney disease (ADPKD), autosomal recessive polycystic kidney disease (ARPKD), kidney diseases associated with cyst formation or cystogenesis, focal segmental glomerulosclerosis (FSGS) and other diseases associated with sclerosis of the kidney, laminopathies, age-related macular degeneration (AMD), diabetic macular edema, diabetic retinopathy, glaucoma, age related retinal disease, immune system senescence, respiratory tract infections, urinary tract infections, heart failure, osteoarthritis, pulmonary arterial hypertension (PAH), and chronic obstructive pulmonary disease (COPD).

16. The method according to claim 13, wherein the mTORC-mediated disease, disorder, or condition is selected from Fragile X syndrome (FXS), amyotrophic lateral sclerosis (ALS), epilepsy, focal cortical dysplasia (FCD), hemimegalencephaly (HME), familial focal epilepsy with variable foci (FFEV), temporal lobe epilepsy (TLE), seizures, neurodegenerative diseases, Down syndrome, Rett syndrome (RTS), and diseases associated with activation or hyperactivation of mTOR signaling in the brain.
